SEQUENCE LISTING (720 sequences)

SEQ ID NO: 1 - protein, 131 aa, synthetic construct
QVQLVQSGGGLVQPGGSLRLSCAASGFTFSSYAMSWVRDIQMTQSPSSQAPGKGLEWVSAIFNSGGSTYYADSVKGRFTVSRDNSKNTLYLQMNSLRAEDTALYYCAKDLGGYNYGLFDYWGQGTLVTVSS

SEQ ID NO: 2 - protein, 7 aa, synthetic construct
GFTFSSY

SEQ ID NO: 3 - protein, 5 aa, synthetic construct
SYAMS

SEQ ID NO: 4 - protein, 6 aa, synthetic construct
FNSGGS

SEQ ID NO: 5 - protein, 17 aa, synthetic construct
AIFNSGGSTYYADSVKG

SEQ ID NO: 6 - protein, 12 aa, synthetic construct
DLGGYNYGLFDY

SEQ ID NO: 7 - protein, 11 aa, synthetic construct
WASQSISSWLA

SEQ ID NO: 8 - protein, 7 aa, synthetic construct
KASSLES

SEQ ID NO: 9 - protein, 9 aa, synthetic construct
QQYNSYSYT

SEQ ID NO: 10 - protein, 107 aa, synthetic construct
DIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGQGTKLEIK

SEQ ID NO: 11 - protein, 121 aa, synthetic construct
EVQLVQSGGGLVQPGGSLRLSCAASGFTFSSYAMSWVRQAPGKGLEWVSAIFNSGGSTYYADSVKGRFTVSRDNSKNTLYLQMNSLRAEDTALYYCAKDLGGYNYGLFDYWGQGTLVTVSS

SEQ ID NO: 12 - protein, 121 aa, synthetic construct
EVQLVQSGGGLVQPGGSLRLSCAASGFTFSSYAMSWVRQAPGKGLEWVSAIFNAGGSTYYADSVKGRFTVSRDNSKNTLYLQMNSLRAEDTALYYCAKDLGGYNYGLFDYWGQGTLVTVSS

SEQ ID NO: 13 - protein, 6 aa, synthetic construct
FNAGGS

SEQ ID NO: 14 - protein, 17 aa, synthetic construct
AIFNAGGSTYYADSVKG

SEQ ID NO: 15 - protein, 121 aa, synthetic construct
EVQLVQSGGGLVQPGGSLRLSCAASGFTFSSYAMSWVRQAPGKGLEWVSAIFNSGGSTYYADAVKGRFTVSRDNSKNTLYLQMNSLRAEDTALYYCAKDLGGYNYGLFDYWGQGTLVTVSS

SEQ ID NO: 16 - protein, 17 aa, synthetic construct
AIFNSGGSTYYADAVKG

SEQ ID NO: 17 - protein, 121 aa, synthetic construct
EVQLVQSGGGLVQPGGSLRLSCAASGFTFSSYAMSWVRQAPGKGLEWVSAIFNSGGSTYYADSVKGRFTVSRDNAKNTLYLQMNSLRAEDTALYYCAKDLGGYNYGLFDYWGQGTLVTVSS

SEQ ID NO: 18 - protein, 121 aa, synthetic construct
EVQLVQSGGGLVQPGGSLRLSCAASGFTFSSYAMSWVRQAPGKGLEWVSAIFNSGGSTYYADSVKGRFTVSRDNSKQTLYLQMNSLRAEDTALYYCAKDLGGYNYGLFDYWGQGTLVTVSS

SEQ ID NO: 19 - protein, 121 aa, synthetic construct
EVQLVQSGGGLVQPGGSLRLSCAASGFTFSSYAMSWVRQAPGKGLEWVSAIFNSGGSTYYADSVKGRFTVSRDNSKNTLYLQMQSLRAEDTALYYCAKDLGGYNYGLFDYWGQGTLVTVSS

SEQ ID NO: 20 - protein, 121 aa, synthetic construct
EVQLVQSGGGLVQPGGSLRLSCAASGFTFSSYAMSWVRQAPGKGLEWVSAIFNAGGSTYYADAVKGRFTVSRDNSKNTLYLQMNSLRAEDTALYYCAKDLGGYNYGLFDYWGQGTLVTVSS

SEQ ID NO: 21 - protein, 17 aa, synthetic construct
AIFNAGGSTYYADAVKG

SEQ ID NO: 22 - protein, 121 aa, synthetic construct
EVQLVQSGGGLVQPGGSLRLSCAASGFTFSSYAMSWVRQAPGKGLEWVSAIFNAGGSTYYADAVKGRFTVSRDNAKNTLYLQMNSLRAEDTALYYCAKDLGGYNYGLFDYWGQGTLVTVSS

SEQ ID NO: 23 - protein, 121 aa, synthetic construct
EVQLVQSGGGLVQPGGSLRLSCAASGFTFSSYAMSWVRQAPGKGLEWVSAIFNAGGSTYYADAVKGRFTVSRDNAKQTLYLQMNSLRAEDTALYYCAKDLGGYNYGLFDYWGQGTLVTVSS

SEQ ID NO: 24 - protein, 121 aa, synthetic construct
EVQLVQSGGGLVQPGGSLRLSCAASGFTFSSYAMSWVRQAPGKGLEWVSAIFNAGGSTYYADAVKGRFTVSRDNAKQTLYLQMQSLRAEDTALYYCAKDLGGYNYGLFDYWGQGTLVTVSS

SEQ ID NO: 25 - protein, 107 aa, synthetic construct
DIRMTQSPSTLSASVGDRVTITCWASQSISSWLAWYQQKPGKAPKLLIYKASSLESGVPSRFSGSGSGTEFTLTISSLQPDNFATYYCQQYNSYSYTFGQGTKLEIK

SEQ ID NO: 26 - protein, 107 aa, synthetic construct
DIRMTQSPSTLSASVGDRVTITCWASQSISSWLAWYQQKPGKAPKLLIYKASSLESGVPSRFSGSGSGTEFTLTISSLQPDDFATYYCQQYNAYSYTFGQGTKLEIK

SEQ ID NO: 27 - protein, 9 aa, synthetic construct
QQYNAYSYT

SEQ ID NO: 28 - protein, 107 aa, synthetic construct
DIRMTQSPSTLSASVGDRVTITCWASQSISSWLAWYQQKPGKAPKLLIYKASSLESGVPSRFSGSGSGTEFTLTISSLQPDNFATYYCQQYNAYSYTFGQGTKLEIK

SEQ ID NO: 29 - protein, 121 aa, synthetic construct
  VARIANT 1: note = E or Q
  VARIANT 54: note = S or A
  VARIANT 63: note = S or A
  VARIANT 75: note = S or A
  VARIANT 77: note = N or Q
  VARIANT 84: note = N or Q
XVQLVQSGGGLVQPGGSLRLSCAASGFTFSSYAMSWVRQAPGKGLEWVSAIFNXGGSTYYADXVKGRFTVSRDNXKXTLYLQMXSLRAEDTALYYCAKDLGGYNYGLFDYWGQGTLVTVSS

SEQ ID NO: 30 - protein, 6 aa, synthetic construct
  VARIANT 3: note = S or A
FNXGGS

SEQ ID NO: 31 - protein, 17 aa, synthetic construct
  VARIANT 5: note = S or A
  VARIANT 14: note = S or A
AIFNXGGSTYYADXVKG

SEQ ID NO: 32 - protein, 107 aa, synthetic construct
  VARIANT 3: note = R or Q
  VARIANT 82: note = N or D
  VARIANT 93: note = S or A
DIXMTQSPSTLSASVGDRVTITCWASQSISSWLAWYQQKPGKAPKLLIYKASSLESGVPSRFSGSGSGTEFTLTISSLQPDXFATYYCQQYNXYSYTFGQGTKLEIK

SEQ ID NO: 33 - protein, 9 aa, synthetic construct
  VARIANT 5: note = S or A
QQYNXYSYT

SEQ ID NO: 34 - protein, 121 aa, synthetic construct
QVQLVQSGGGLVQPGGSQRLSCAASGFTFTSYAMSWVRQAPGKGLEWVSAISGTGDSTFYADSVKGRFTFSRDNSKNTLYLQMNSLRAEDTAVYYCAKDLGWLQYGLFDYWGQGTLVTVSS

SEQ ID NO: 35 - protein, 7 aa, synthetic construct
GFTFTSY

SEQ ID NO: 36 - protein, 6 aa, synthetic construct
SGTGDS

SEQ ID NO: 37 - protein, 17 aa, synthetic construct
AISGTGDSTFYADSVKG

SEQ ID NO: 38 - protein, 12 aa, synthetic construct
DLGWLQYGLFDY

SEQ ID NO: 39 - protein, 108 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGGGTKVEIK

SEQ ID NO: 40 - protein, 12 aa, synthetic construct
RASQSVSSSYLA

SEQ ID NO: 41 - protein, 7 aa, synthetic construct
GASSRAT

SEQ ID NO: 42 - protein, 9 aa, synthetic construct
QQYNNWPLT

SEQ ID NO: 43 - protein, 121 aa, synthetic construct
EVQLVQSGGGLVQPGGSQRLSCAASGFTFTSYAMSWVRQAPGKGLEWVSAISGTGDSTFYADSVKGRFTFSRDNSKNTLYLQMNSLRAEDTAVYYCAKDLGWLQYGLFDYWGQGTLVTVSS

SEQ ID NO: 44 - protein, 121 aa, synthetic construct
EVQLVQSGGGLVQPGGSQRLSCAASGFTFTSYAMSWVRQAPGKGLEWVSAISGTGESTFYADSVKGRFTFSRDNSKNTLYLQMNSLRAEDTAVYYCAKDLGWLQYGLFDYWGQGTLVTVSS

SEQ ID NO: 45 - protein, 6 aa, synthetic construct
SGTGES

SEQ ID NO: 46 - protein, 17 aa, synthetic construct
AISGTGESTFYADSVKG

SEQ ID NO: 47 - protein, 121 aa, synthetic construct
EVQLVQSGGGLVQPGGSQRLSCAASGFTFTSYAMSWVRQAPGKGLEWVSAISGTGDSTFYADAVKGRFTFSRDNSKNTLYLQMNSLRAEDTAVYYCAKDLGWLQYGLFDYWGQGTLVTVSS

SEQ ID NO: 48 - protein, 17 aa, synthetic construct
AISGTGDSTFYADAVKG

SEQ ID NO: 49 - protein, 121 aa, synthetic construct
EVQLVQSGGGLVQPGGSQRLSCAASGFTFTSYAMSWVRQAPGKGLEWVSAISGTGDSTFYADSVKGRFTFSRENSKNTLYLQMNSLRAEDTAVYYCAKDLGWLQYGLFDYWGQGTLVTVSS

SEQ ID NO: 50 - protein, 121 aa, synthetic construct
QVQLVQSGGGLVQPGGSQRLSCAASGFTFTSYAMSWVRQAPGKGLEWVSAISGTGDSTFYADSVKGRFTFSRDNAKNTLYLQMNSLRAEDTAVYYCAKDLGWLQYGLFDYWGQGTLVTVSS

SEQ ID NO: 51 - protein, 121 aa, synthetic construct
EVQLVQSGGGLVQPGGSQRLSCAASGFTFTSYAMSWVRQAPGKGLEWVSAISGTGDSTFYADSVKGRFTFSRDNSKNTLYLQMNALRAEDTAVYYCAKDLGWLQYGLFDYWGQGTLVTVSS

SEQ ID NO: 52 - protein, 121 aa, synthetic construct
  VARIANT 1: note = E or Q
  VARIANT 18: note = Q or L
  VARIANT 56: note = D or E
  VARIANT 63: note = S or A
  VARIANT 73: note = D or E
  VARIANT 75: note = S or A
  VARIANT 85: note = S or A
XVQLVQSGGGLVQPGGSXRLSCAASGFTFTSYAMSWVRQAPGKGLEWVSAISGTGXSTFYADXVKGRFTFSRXNXKNTLYLQMNXLRAEDTAVYYCAKDLGWLQYGLFDYWGQGTLVTVSS

SEQ ID NO: 53 - protein, 6 aa, synthetic construct
  VARIANT 5: note = D or E
SGTGXS

SEQ ID NO: 54 - protein, 17 aa, synthetic construct
  VARIANT 7: note = D or E
  VARIANT 14: note = S or A
AISGTGXSTFYADXVKG

SEQ ID NO: 55 - protein, 124 aa, synthetic construct
QVQLQESGPGLVRPSGTLSLTCAVSGGSISSPTWWSWVRQPPGKGLEWIGEIHPSGRTNYNPSLKSRVTISVDKSKNQFSLKLGSVTAADTAVYYCAREGFYYGSGNYYYFDYWGQGTLVTVSS

SEQ ID NO: 56 - protein, 8 aa, synthetic construct
GGSISSPT

SEQ ID NO: 57 - protein, 6 aa, synthetic construct
SPTWWS

SEQ ID NO: 58 - protein, 5 aa, synthetic construct
HPSGR

SEQ ID NO: 59 - protein, 16 aa, synthetic construct
EIHPSGRTNYNPSLKS

SEQ ID NO: 60 - protein, 15 aa, synthetic construct
EGFYYGSGNYYYFDY

SEQ ID NO: 61 - protein, 106 aa, synthetic construct
DIVMTQTPATLSASVGDRVTITCRASQSVRSNLAWYQQKPGQAPRLLIYGASTRATGIPARFSGSGSGTEFTLTISSLQSEDFAVYYCQQYNNWPTFGQGTRLEIK

SEQ ID NO: 62 - protein, 11 aa, synthetic construct
RASQSVRSNLA

SEQ ID NO: 63 - protein, 7 aa, synthetic construct
GASTRAT

SEQ ID NO: 64 - protein, 8 aa, synthetic construct
QQYNNWPT

SEQ ID NO: 65 - protein, 124 aa, synthetic construct
EVQLQESGPGLVRPSGTLSLTCAVSGGSISSPTWWSWVRQPPGKGLEWIGEIHPSGRTNYNPSLKSRVTISVDKSKNQFSLKLGSVTAADTAVYYCAREGFYYGSGNYYYFDYWGQGTLVTVSS

SEQ ID NO: 66 - protein, 124 aa, synthetic construct
  VARIANT 1: note = Q or E
XVQLQESGPGLVRPSGTLSLTCAVSGGSISSPTWWSWVRQPPGKGLEWIGEIHPSGRTNYNPSLKSRVTISVDKSKNQFSLKLGSVTAADTAVYYCAREGFYYGSGNYYYFDYWGQGTLVTVSS

SEQ ID NO: 67 - protein, 126 aa, synthetic construct
QVQLVESGGDVVQPGRSLRLSCAASGFILSNYGMHWVRQAPGKGLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 68 - protein, 7 aa, synthetic construct
GFILSNY

SEQ ID NO: 69 - protein, 5 aa, synthetic construct
NYGMH

SEQ ID NO: 70 - protein, 6 aa, synthetic construct
WYDGSN

SEQ ID NO: 71 - protein, 17 aa, synthetic construct
AMWYDGSNNYYEDSVKG

SEQ ID NO: 72 - protein, 17 aa, synthetic construct
ERVSRHFDWHYYYGMDV

SEQ ID NO: 73 - protein, 112 aa, synthetic construct
EIVMTQSPLSLPVTPGEPASISCRSSQSLLHSNGYNYLDWYLQKPGQSPQLLISLGSIRASGVPDRFSGSGSGTNFTLTISRVEAEDVGFYYCMQALQTPRTFGQGTKVDIT

SEQ ID NO: 74 - protein, 16 aa, synthetic construct
RSSQSLLHSNGYNYLD

SEQ ID NO: 75 - protein, 7 aa, synthetic construct
LGSIRAS

SEQ ID NO: 76 - protein, 9 aa, synthetic construct
MQALQTPRT

SEQ ID NO: 77 - protein, 126 aa, synthetic construct
EVQLVESGGDVVQPGRSLRLSCAASGFILSNYGMHWVRQAPGKGLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 78 - protein, 126 aa, synthetic construct
EVQLVESGGDVVQPGRSLRLSCAASGFILSNYGMHWVRQAPGKGLEWVAAMWYDGSNNYYEDTVKGRFTISRDNSKNTLYLQMNSLRAEDTAVYYCARERVSRHFDWHYYYGMDVWGQGTTVTVSS

SEQ ID NO: 79 - protein, 17 aa, synthetic construct
AMWYDGSNNYYEDTVKG

SEQ ID NO: 80 - protein, 126 aa, synthetic construct
EVQLVESGGDVVQPGRSLRLSCAASGFILSNYGMHWVRQAPGKGLEWVAAMWYDGSNNYYEDSVKGRFTISRDQSKNTLYLQMNSLRAEDTAVYYCARERVSRHFDWHYYYGMDVWGQGTTVTVSS

SEQ ID NO: 81 - protein, 126 aa, synthetic construct
EVQLVESGGDVVQPGRSLRLSCAASGFILSNYGMHWVRQAPGKGLEWVAAMWYDGSNNYYEDSVKGRFTISRDNSKQTLYLQMNSLRAEDTAVYYCARERVSRHFDWHYYYGMDVWGQGTTVTVSS

SEQ ID NO: 82 - protein, 126 aa, synthetic construct
QVQLVESGGDVVQPGRSLRLSCAASGFILSNYGMHWVRQAPGKGLEWVAAMWYDGSNNYYEDSVKGRFTISRDNSKNTLYLQMQSLRAEDTAVYYCARERVSRHFDWHYYYGMDVWGQGTTVTVSS

SEQ ID NO: 83 - protein, 126 aa, synthetic construct
EVQLVESGGDVVQPGRSLRLSCAASGFILSNYGMHWVRQAPGKGLEWVAAMWYDGSNNYYEDTVKGRFTISRDQSKQTLYLQMQSLRAEDTAVYYCARERVSRHFDWHYYYGMDVWGQGTTVTVSS

SEQ ID NO: 84 - protein, 112 aa, synthetic construct
EIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGQGTKVDIT

SEQ ID NO: 85 - protein, 112 aa, synthetic construct
EIVMTQSPLSLPVTPGEPASISCRSSQSLLHSNGYNYLDWYLQKPGQSPQLLISLGSIRASGVPDRFSGSGSGTQFTLTISRVEAEDVGFYYCMQALQTPRTFGQGTKVDIT

SEQ ID NO: 86 - protein, 112 aa, synthetic construct
EIVMTQSPLSLPVTPGEPASISCRSSQSLLHSNGYNYLDWYLQKPGQSPQLLISLGSIRASGVPDRFSGSGSGTNFALTISRVEAEDVGFYYCMQALQTPRTFGQGTKVDIT

SEQ ID NO: 87 - protein, 126 aa, synthetic construct
  VARIANT 1: note = E or Q
  VARIANT 63: note = S or T
  VARIANT 74: note = N or Q
  VARIANT 77: note = N or Q
  VARIANT 84: note = N or Q
XVQLVESGGDVVQPGRSLRLSCAASGFILSNYGMHWVRQAPGKGLEWVAAMWYDGSNNYYEDXVKGRFTISRDXSKXTLYLQMXSLRAEDTAVYYCARERVSRHFDWHYYYGMDVWGQGTTVTVSS

SEQ ID NO: 88 - protein, 17 aa, synthetic construct
  VARIANT 14: note = S or T
AMWYDGSNNYYEDXVKG

SEQ ID NO: 89 - protein, 112 aa, synthetic construct
  VARIANT 75: note = N, D or Q
  VARIANT 77: note = T or A
EIVMTQSPLSLPVTPGEPASISCRSSQSLLHSNGYNYLDWYLQKPGQSPQLLISLGSIRASGVPDRFSGSGSGTXFXLTISRVEAEDVGFYYCMQALQTPRTFGQGTKVDIT

SEQ ID NO: 90 - protein, 126 aa, synthetic construct
QVQLVESGGDVVQPGRSLRLSCAASGFTLSSYGMHWVRQAPGKGLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 91 - protein, 7 aa, synthetic construct
GFTLSSY

SEQ ID NO: 92 - protein, 5 aa, synthetic construct
SYGMH

SEQ ID NO: 93 - protein, 112 aa, synthetic construct
EIVMTQSPLSLPVTPGEPASISCRSSQSLLHYNGYNYLDWYLQKPGQSPQLLISLGSIRASGVPDRFSGSGSGTNFTLTISRVEAEDVGFYYCMQALQTPRTFGQGTKVDIT

SEQ ID NO: 94 - protein, 16 aa, synthetic construct
RSSQSLLHYNGYNYLD

SEQ ID NO: 95 - protein, 112 aa, synthetic construct
EIVMTQSPLSLPVTPGEPASISCRSSQSLLHYNGYNYLDWYLQKPGQSPQLLISLGSIRASGVPDRFSGSGSGTDFTLTISRVEAEDVGFYYCMQALQTPRTFGQGTKVDIT

SEQ ID NO: 96 - protein, 112 aa, synthetic construct
EIVMTQSPLSLPVTPGEPASISCRSSQSLLHYNGYNYLDWYLQKPGQSPQLLISLGSIRASGVPDRFSGSGSGTQFTLTISRVEAEDVGFYYCMQALQTPRTFGQGTKVDIT

SEQ ID NO: 97 - protein, 112 aa, synthetic construct
EIVMTQSPLSLPVTPGEPASISCRSSQSLLHYNGYNYLDWYLQKPGQSPQLLISLGSIRASGVPDRFSGSGSGTNFALTISRVEAEDVGFYYCMQALQTPRTFGQGTKVDIT

SEQ ID NO: 98 - protein, 126 aa, synthetic construct
  VARIANT 1: note = E or Q
  VARIANT 63: note = S or T
  VARIANT 74: note = N or Q
  VARIANT 77: note = N or Q
  VARIANT 84: note = N or Q
XVQLVESGGDVVQPGRSLRLSCAASGFTLSSYGMHWVRQAPGKGLEWVAAMWYDGSNNYYEDXVKGRFTISRDXSKXTLYLQMXSLRAEDTAVYYCARERVSRHFDWHYYYGMDVWGQGTTVTVSS

SEQ ID NO: 99 - protein, 112 aa, synthetic construct
  VARIANT 75: note = N, D or Q
  VARIANT 77: note = A or T
EIVMTQSPLSLPVTPGEPASISCRSSQSLLHYNGYNYLDWYLQKPGQSPQLLISLGSIRASGVPDRFSGSGSGTXFXLTISRVEAEDVGFYYCMQALQTPRTFGQGTKVDIT

SEQ ID NO: 100 - protein, 118 aa, synthetic construct
EVQLQQSAAELARPGASVKMSCKASGYTFTAYTIHWVKQRPGQGLEWIGYINPSSGYTEYNQKFKDKTTLTADQSSPTAYIQLSTLTSEDSAVYYCTREGGLLWFDYWGQGTTLTVST

SEQ ID NO: 101 - protein, 7 aa, synthetic construct
GYTFTAY

SEQ ID NO: 102 - protein, 5 aa, synthetic construct
AYTIH

SEQ ID NO: 103 - protein, 6 aa, synthetic construct
NPSSGY

SEQ ID NO: 104 - protein, 17 aa, synthetic construct
YINPSSGYTEYNQKFKD

SEQ ID NO: 105 - protein, 9 aa, synthetic construct
EGGLLWFDY

SEQ ID NO: 106 - protein, 113 aa, synthetic construct
DIVMSQSPSSLAVSVGEKVTMSCKSSHSLLYGNFQNNYLAWYQQKPGQSPKLLIYWASTRESGVPDRFTGSGSGTDFTLTISSVKAEDLAVYYCQQYYSYPYTFGGGTKLEIK

SEQ ID NO: 107 - protein, 17 aa, synthetic construct
KSSHSLLYGNFQNNYLA

SEQ ID NO: 108 - protein, 7 aa, synthetic construct
WASTRES

SEQ ID NO: 109 - protein, 9 aa, synthetic construct
QQYYSYPYT

SEQ ID NO: 110 - protein, 118 aa, synthetic construct
QVQLQQSAAELARPGASVKMSCKASGYTFTAYTIHWVKQRPGQGLEWIGYINPSSGYTEYNQKFKDKTTLTADQSSPTAYIQLSTLTSEDSAVYYCTREGGLLWFDYWGQGTTLTVST

SEQ ID NO: 111 - protein, 118 aa, synthetic construct
  VARIANT 1: note = E or Q
XVQLQQSAAELARPGASVKMSCKASGYTFTAYTIHWVKQRPGQGLEWIGYINPSSGYTEYNQKFKDKTTLTADQSSPTAYIQLSTLTSEDSAVYYCTREGGLLWFDYWGQGTTLTVST

SEQ ID NO: 112 - protein, 118 aa, synthetic construct
QVQLVQSGAEVKKPGASVKMSCKASGYTFTAYTIHWVRQAPGQGLEWIGYINPSSGYTEYNQKFKDRTTLTADTSIPTAYMELSRLRSDDTAVYYCTREGGLLWFDYWGQGTLVTVSS

SEQ ID NO: 113 - protein, 118 aa, synthetic construct
QVQLVQSGAEVKKPGASVKMSCKASGYTFTAYTIHWVRQAPGQGLEWIGYINPSSGYTEYNQKFKDRTTLTADTSIPTAYIELSRLRSDDTAVYYCTREGGLLWFDYWGQGTLVTVSS

SEQ ID NO: 114 - protein, 118 aa, synthetic construct
EVQLVQSGAEVKKPGASVKVSCKASGYTFTAYTIHWVRQAPGQGLEWMGYINPSSGYTEYNQKFKDRTTLTADTSIPTAYMELSRLRSDDTAVYYCTREGGLLWFDYWGQGTLVTVSS

SEQ ID NO: 115 - protein, 118 aa, synthetic construct
EVQLVQSGAEVKKPGASVKVSCKASGYTFTAYTIHWVRQAPGQGLEWMGYINPSSGYTEYNQKFKDRTTLTADTSIPTAYMELSRLRSQDTAVYYCTREGGLLWFDYWGQGTLVTVSS

SEQ ID NO: 116 - protein, 118 aa, synthetic construct
EVQLVQSGAEVKKPGASVKMSCKASGYTFTAYTIHWVRQAPGQGLEWIGYINPSSGYTEYNQKFKDRTTLTADTSIPTAYIELSRLRSDDTAVYYCTREGGLLWFDYWGQGTLVTVSS

SEQ ID NO: 117 - protein, 118 aa, synthetic construct
EVQLVQSGAEVKKPGASVKMSCKASGYTFTAYTIHWVRQAPGQGLEWIGYINPSSGYTEYNQKFKDRTTLTADTSIPTAYIELSRLRSQDTAVYYCTREGGLLWFDYWGQGTLVTVSS

SEQ ID NO: 118 - protein, 118 aa, synthetic construct
  VARIANT 1: note = E or Q
  VARIANT 20: note = M or V
  VARIANT 48: note = I or M
  VARIANT 81: note = I or M
  VARIANT 89: note = D or Q
XVQLVQSGAEVKKPGASVKXSCKASGYTFTAYTIHWVRQAPGQGLEWXGYINPSSGYTEYNQKFKDRTTLTADTSIPTAYXELSRLRSXDTAVYYCTREGGLLWFDYWGQGTLVTVSS

SEQ ID NO: 119 - protein, 113 aa, synthetic construct
DIVMTQSPDSLAVSLGERATINCKSSHSLLYGNFQNNYLAWYQQKPGQPPKLLIYWASTRESGVPDRFSGSGSGTDFTLTISSLQAEDVAVYYCQQYYSYPYTFGGGTKLEIK

SEQ ID NO: 120 - protein, 120 aa, synthetic construct
QVQLQESGPGLVKPSGTLSLTCAVSGGSISSSDWWTWVRQPPGKGLEWIGEIYHSGSTNYNPSLKSRVTISVDKSKNQFSLNLNSVTAADTAVYYCARGYSGSYFDLDIWGQGTTVTVSS

SEQ ID NO: 121 - protein, 8 aa, synthetic construct
GGSISSSD

SEQ ID NO: 122 - protein, 6 aa, synthetic construct
SSDWWT

SEQ ID NO: 123 - protein, 5 aa, synthetic construct
YHSGS

SEQ ID NO: 124 - protein, 16 aa, synthetic construct
EIYHSGSTNYNPSLKS

SEQ ID NO: 125 - protein, 11 aa, synthetic construct
GYSGSYFDLDI

SEQ ID NO: 126 - protein, 107 aa, synthetic construct
EIVMTQSPSSLSASVGDRVTITCRASQGISNYLAWYQQKPGKVPKLLIYAASTLQSGVPSRFSGSGSGTEFTLTISSLQPEDFATYYCQQLNSYPLTFGGGTKVEIK

SEQ ID NO: 127 - protein, 11 aa, synthetic construct
RASQGISNYLA

SEQ ID NO: 128 - protein, 7 aa, synthetic construct
AASTLQS

SEQ ID NO: 129 - protein, 9 aa, synthetic construct
QQLNSYPLT

SEQ ID NO: 130 - protein, 107 aa, synthetic construct
DIRLTQSPSFLSASVGDRVTITCRASQGISSYLAWYQQKPGKAPKLLIYAASTLQSGVPSRFSGSGSGTEFTLTISSLQPEDFATYYCQQLNSYPFTFGPGTKVDIK

SEQ ID NO: 131 - protein, 11 aa, synthetic construct
RASQGISSYLA

SEQ ID NO: 132 - protein, 9 aa, synthetic construct
QQLNSYPFT

SEQ ID NO: 133 - protein, 107 aa, synthetic construct
DIRLTQSPSFLSASVGDRVTITCRASQGISSYLAWYQQKPGKAPKLLIYAASTLQSGVPSRFSGSGSGTEFTLTISSLQPEDFATYYCQQLNAYPFTFGPGTKVDIK

SEQ ID NO: 134 - protein, 9 aa, synthetic construct
QQLNAYPFT

SEQ ID NO: 135 - protein, 120 aa, synthetic construct
QVQLVQSGPGLVKPSGTLSLTCAVSGGSISSSDWWTWVRQPPGKGLEWIGEIYHSGSTNYNPSLKSRVTISVDKSKNQFSLKLNSVTAADTAVYYCARGSSGSYFDLDIWGQGTTVTVSS

SEQ ID NO: 136 - protein, 107 aa, synthetic construct
DIVMTQSPSTLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 137 - protein, 120 aa, synthetic construct
  VARIANT 1: note = E or Q
  VARIANT 5: note = Q or V
  VARIANT 6: note = E or Q
  VARIANT 82: note = K or N
  VARIANT 85: note = S or A
  VARIANT 100: note = S or Y
XVQLXXSGPGLVKPSGTLSLTCAVSGGSISSSDWWTWVRQPPGKGLEWIGEIYHSGSTNYNPSLKSRVTISVDKSKNQFSLXLNXVTAADTAVYYCARGXSGSYFDLDIWGQGTTVTVSS

SEQ ID NO: 138 - protein, 11 aa, synthetic construct
  VARIANT 2: note = S or Y
GXSGSYFDLDI

SEQ ID NO: 139 - protein, 107 aa, synthetic construct
  VARIANT 1: note = E or D
  VARIANT 10: note = T or S
  VARIANT 31: note = N or S
  VARIANT 43: note = V or A
  VARIANT 93: note = S or A
XIVMTQSPSXLSASVGDRVTITCRASQGISXYLAWYQQKPGKXPKLLIYAASTLQSGVPSRFSGSGSGTEFTLTISSLQPEDFATYYCQQLNXYPLTFGGGTKVEIK

SEQ ID NO: 140 - protein, 11 aa, synthetic construct
  VARIANT 8: note = N or S
RASQGISXYLA

SEQ ID NO: 141 - protein, 9 aa, synthetic construct
  VARIANT 5: note = S or A
QQLNXYPLT

SEQ ID NO: 142 - protein, 107 aa, synthetic construct
  VARIANT 93: note = S or A
DIRLTQSPSFLSASVGDRVTITCRASQGISSYLAWYQQKPGKAPKLLIYAASTLQSGVPSRFSGSGSGTEFTLTISSLQPEDFATYYCQQLNXYPFTFGPGTKVDIK

SEQ ID NO: 143 - protein, 9 aa, synthetic construct
  VARIANT 5: note = S or A
QQLNXYPFT

SEQ ID NO: 144 - protein, 120 aa, synthetic construct
QVQLQESGPGLVKPSGTLSLTCAVSGGSISSSKWWSWVRQSPGKGLEWIGEIFHSGSINHNTSFKSRVTISVDKSKNQFSLKLSSVTAADTAVYYCARGGSGSYDAFDIWGQGTMVTVSS

SEQ ID NO: 145 - protein, 8 aa, synthetic construct
GGSISSSK

SEQ ID NO: 146 - protein, 6 aa, synthetic construct
SSKWWS

SEQ ID NO: 147 - protein, 5 aa, synthetic construct
FHSGS

SEQ ID NO: 148 - protein, 16 aa, synthetic construct
EIFHSGSINHNTSFKS

SEQ ID NO: 149 - protein, 11 aa, synthetic construct
GGSGSYDAFDI

SEQ ID NO: 150 - protein, 107 aa, synthetic construct
DIQVTQSPATLSVSPGERVTLSCRASRSVRSNLAWYQQKPGQAPRLLIYGASSRATGIPDRFSGSGSGTDFTLTISSLQPDDFAVYYCQQYNNWPITFGQGTRLEIK

SEQ ID NO: 151 - protein, 11 aa, synthetic construct
RASRSVRSNLA

SEQ ID NO: 152 - protein, 9 aa, synthetic construct
QQYNNWPIT

SEQ ID NO: 153 - protein, 120 aa, synthetic construct
  VARIANT 1: note = Q or E
  VARIANT 61: note = N or Q
  VARIANT 63: note = S or A
XVQLQESGPGLVKPSGTLSLTCAVSGGSISSSKWWSWVRQSPGKGLEWIGEIFHSGSINHXTXFKSRVTISVDKSKNQFSLKLSSVTAADTAVYYCARGGSGSYDAFDIWGQGTMVTVSS

SEQ ID NO: 154 - protein, 16 aa, synthetic construct
  VARIANT 11: note = N or Q
  VARIANT 13: note = S or A
EIFHSGSINHXTXFKS

SEQ ID NO: 155 - protein, 122 aa, synthetic construct
QVQLQESGPGLVKPSGTLSLTCAVSGGSISSSDWWTWVRQPPGKGLEWIGEIYHSGSTNYNPSLKSRVTISVDKSKNQFSLNLNSVTAADTAVYYCARGYSGSYFDLDIWGQGTTVTVSSGS

SEQ ID NO: 156 - protein, 107 aa, synthetic construct
EIVLTQSPSFLSASVGDRVTITCRASQGISSYLAWYQQKPGKAPKLLIYAASTLQSGVPSRFSGSGSGTEFTLTISSLQPEDFATYYCQQLNSYPFTFGPGTKLEIK

SEQ ID NO: 157 - protein, 107 aa, synthetic construct
EIVLTQSPSFLSASVGDRVTITCRASQGISSYLAWYQQKPGKAPKLLIYAASTLQSGVPSRFSGSGSGTEFTLTISSLQPEDFATYYCQQLNAYPFTFGPGTKLEIK

SEQ ID NO: 158 - protein, 122 aa, synthetic construct
  VARIANT 1: note = Q or E
  VARIANT 85: note = S or A
XVQLQESGPGLVKPSGTLSLTCAVSGGSISSSDWWTWVRQPPGKGLEWIGEIYHSGSTNYNPSLKSRVTISVDKSKNQFSLNLNXVTAADTAVYYCARGYSGSYFDLDIWGQGTTVTVSSGS

SEQ ID NO: 159 - protein, 107 aa, synthetic construct
  VARIANT 93: note = S or A
EIVLTQSPSFLSASVGDRVTITCRASQGISSYLAWYQQKPGKAPKLLIYAASTLQSGVPSRFSGSGSGTEFTLTISSLQPEDFATYYCQQLNXYPFTFGPGTKLEIK

SEQ ID NO: 160 - protein, 122 aa, synthetic construct
QVQLVESGGGLVQPGGSLRLSCAASGFTFSSYAMSWVRQAPGKGLEWVSEISGSGDRTSYADSVKGRFTISRDNSKNRLYLQMNRLRTEDTAVYYCAKDLGPSGWYGLFDYWGQGTLVTVSS

SEQ ID NO: 161 - protein, 6 aa, synthetic construct
SGSGDR

SEQ ID NO: 162 - protein, 17 aa, synthetic construct
EISGSGDRTSYADSVKG

SEQ ID NO: 163 - protein, 13 aa, synthetic construct
DLGPSGWYGLFDY

SEQ ID NO: 164 - protein, 108 aa, synthetic construct
DIRMTQSPSTLSASVGDRVTITCRASQSISSWLAWYQQKPGKAPKLLIYKASSLESGVPSRFSGSGSGTEFTLTISSLQPDDFATYYCQQYNSYSTWTFGQGTKVDIK

SEQ ID NO: 165 - protein, 11 aa, synthetic construct
RASQSISSWLA

SEQ ID NO: 166 - protein, 10 aa, synthetic construct
QQYNSYSTWT

SEQ ID NO: 167 - protein, 108 aa, synthetic construct
DIRMTQSPSTLSASVGDRVTITCRASQSISSWLAWYQQKPGKAPKLLIYKASSLESGVPSRFSGSGSGTEFTLTISSLQPDDFATYYCQQYNAYSTWTFGQGTKVDIK

SEQ ID NO: 168 - protein, 10 aa, synthetic construct
QQYNAYSTWT

SEQ ID NO: 169 - protein, 122 aa, synthetic construct
  VARIANT 1: note = Q or E
  VARIANT 63: note = S or A
  VARIANT 74: note = N or A
  VARIANT 75: note = S or A
XVQLVESGGGLVQPGGSLRLSCAASGFTFSSYAMSWVRQAPGKGLEWVSEISGSGDRTSYADXVKGRFTISRDXXKNRLYLQMNRLRTEDTAVYYCAKDLGPSGWYGLFDYWGQGTLVTVSS

SEQ ID NO: 170 - protein, 17 aa, synthetic construct
  VARIANT 14: note = S or A
EISGSGDRTSYADXVKG

SEQ ID NO: 171 - protein, 108 aa, synthetic construct
  VARIANT 93: note = S or A
DIRMTQSPSTLSASVGDRVTITCRASQSISSWLAWYQQKPGKAPKLLIYKASSLESGVPSRFSGSGSGTEFTLTISSLQPDDFATYYCQQYNXYSTWTFGQGTKVDIK

SEQ ID NO: 172 - protein, 10 aa, synthetic construct
  VARIANT 5: note = S or A
QQYNXYSTWT

SEQ ID NO: 173 - protein, 120 aa, synthetic construct
EVQLQESGAELVRSGASVKLSCTASDFNIKDSYMHWVTQRPEQGLEWIGWIDPENGDTEYAPKFQGKATMTADTSSNTAYLHLSSLTSEDTAVYYCNVITTVVNYAMDYWGQGTSVTVSS

SEQ ID NO: 174 - protein, 7 aa, synthetic construct
DFNIKDS

SEQ ID NO: 175 - protein, 5 aa, synthetic construct
DSYMH

SEQ ID NO: 176 - protein, 6 aa, synthetic construct
DPENGD

SEQ ID NO: 177 - protein, 17 aa, synthetic construct
WIDPENGDTEYAPKFQG

SEQ ID NO: 178 - protein, 11 aa, synthetic construct
ITTVVNYAMDY

SEQ ID NO: 179 - protein, 106 aa, synthetic construct
QIVLSQSPAIMSASPGEKVTITCSTSSSVSYMHWFQQKPGTSPKLWIYSTSNLASGVPARFSGSGSGTSYSLTISRMEAEDAATYYCQQRSNYPLTFGAGTKLELK

SEQ ID NO: 180 - protein, 10 aa, synthetic construct
STSSSVSYMH

SEQ ID NO: 181 - protein, 7 aa, synthetic construct
STSNLAS

SEQ ID NO: 182 - protein, 9 aa, synthetic construct
QQRSNYPLT

SEQ ID NO: 183 - protein, 120 aa, synthetic construct
EVQLVQSGAEVVKPGATVKLSCKASDFNIKDSYMHWVQQAPGKGLEWIGWIDPENGDTEYAPKFQGRATITADTSTDTAYLELSSLRSEDTAVYYCNVITTVVNYAMDYWGQGTLVTVSS

SEQ ID NO: 184 - protein, 106 aa, synthetic construct
EIVLTQSPATLSASPGERATLSCSTSSSVSYMHWFQQKPGQAPRLWIYSTSNLASGVPARFSGSGSGTDYTLTISSLEPEDFAVYYCQQRSNYPLTFGAGTKVEIK

SEQ ID NO: 185 - protein, 120 aa, synthetic construct
EVQLVQSGAEVVKPGATVKLSCKASDFNIKDSYMHWVQQAPGKGLEWIGWIDPESGDTEYAPKFQGRATITADTSTDTAYLELSSLRSEDTAVYYCNVITTVVNYAMDYWGQGTLVTVSS

SEQ ID NO: 186 - protein, 6 aa, synthetic construct
DPESGD

SEQ ID NO: 187 - protein, 17 aa, synthetic construct
WIDPESGDTEYAPKFQG

SEQ ID NO: 188 - protein, 120 aa, synthetic construct
EVQLQQSGAEVVKPGATVKLSCKASDFNIKDSYMHWVTQAPGKGLEWIGWIDPENGDTEYAPKFQGRATMTADTSTDTAYLELSSLRSEDTAVYYCNVITTVVNYAMDYWGQGTLVTVSS

SEQ ID NO: 189 - protein, 120 aa, synthetic construct
EVQLQQSGAEVVKPGATVKLSCKASDFNIKDSYMHWVTQAPGKGLEWIGWIDPESGDTEYAPKFQGRATMTADTSTDTAYLELSSLRSEDTAVYYCNVITTVVNYAMDYWGQGTLVTVSS

SEQ ID NO: 190 - protein, 120 aa, synthetic construct
EVQLQQSGAEVVKPGATVKLSCKASDFNIKDSYMHWVTQAPGKGLEWIGWIDPEQGDTEYAPKFQGRATMTADTSTDTAYLELSSLRSEDTAVYYCNVITTVVNYAMDYWGQGTLVTVSS

SEQ ID NO: 191 - protein, 6 aa, synthetic construct
DPEQGD

SEQ ID NO: 192 - protein, 17 aa, synthetic construct
WIDPEQGDTEYAPKFQG

SEQ ID NO: 193 - protein, 120 aa, synthetic construct
  VARIANT 5: note = Q or V
  VARIANT 32: note = S or A
  VARIANT 38: note = T or Q
  VARIANT 55: note = Q, S or N
  VARIANT 70: note = M or I
EVQLXQSGAEVVKPGATVKLSCKASDFNIKDXYMHWVXQAPGKGLEWIGWIDPEXGDTEYAPKFQGRATXTADTSTDTAYLELSSLRSEDTAVYYCNVITTVVNYAMDYWGQGTLVTVSS

SEQ ID NO: 194 - protein, 7 aa, synthetic construct
  VARIANT 7: note = S or A
DFNIKDX

SEQ ID NO: 195 - protein, 5 aa, synthetic construct
  VARIANT 2: note = S or A
DXYMH

SEQ ID NO: 196 - protein, 6 aa, synthetic construct
  VARIANT 4: note = Q, S or N
DPEXGD

SEQ ID NO: 197 - protein, 17 aa, synthetic construct
  VARIANT 6: note = Q, S or N
WIDPEXGDTEYAPKFQG

SEQ ID NO: 198 - protein, 120 aa, synthetic construct
EVQLVQSGAEVVKPGATVKLSCKASDFNIKDSYMHWVQQAPGKGLEWIGWIDPEQGDTEYAPKFQGRATITADTSTDTAYLELSSLRSEDTAVYYCNVITTVVNYAMDYWGQGTLVTVSS

SEQ ID NO: 199 - protein, 120 aa, synthetic construct
EVQLVQSGAEVVKPGATVKLSCKASDFNIKDAYMHWVQQAPGKGLEWIGWIDPEQGDTEYAPKFQGRATITADTSTDTAYLELSSLRSEDTAVYYCNVITTVVNYAMDYWGQGTLVTVSS

SEQ ID NO: 200 - protein, 7 aa, synthetic construct
DFNIKDA

SEQ ID NO: 201 - protein, 5 aa, synthetic construct
DAYMH

SEQ ID NO: 202 - protein, 120 aa, synthetic construct
  VARIANT 32: note = S or A
EVQLVQSGAEVVKPGATVKLSCKASDFNIKDXYMHWVQQAPGKGLEWIGWIDPEQGDTEYAPKFQGRATITADTSTDTAYLELSSLRSEDTAVYYCNVITTVVNYAMDYWGQGTLVTVSS

SEQ ID NO: 203 - protein, 15 aa, synthetic construct
DGREVWRYYHYGMDV

SEQ ID NO: 204 - protein, 7 aa, synthetic construct
GFMFSNF

SEQ ID NO: 205 - protein, 5 aa, synthetic construct
NFAMH

SEQ ID NO: 206 - protein, 17 aa, synthetic construct
VIWYDGSNKFYADSVKG

SEQ ID NO: 207 - protein, 15 aa, synthetic construct
DGREVWRYYHYGMDV

SEQ ID NO: 208 - protein, 107 aa, synthetic construct
EIVLTQSPSTLSASVGDRVTITCRASQSISNWLAWYQQKPGKAPKLLIYKASSLESGVPSRFSGSGSGTEFTLTISSLQPDDFATYYCQQYNSYSYTFGQGTKVEIK

SEQ ID NO: 209 - protein, 11 aa, synthetic construct
RASQSISNWLA

SEQ ID NO: 210 - protein, 107 aa, synthetic construct
EIVLTQSPSTLSASVGDRVTITCRASQSISNWLAWYQQKPGKAPKLLIYKASSLESGVPSRFSGSGSGTEFTLTISSLQPDDFATYYCQQYNAYSYTFGQGTKVEIK

SEQ ID NO: 211 - protein, 124 aa, synthetic construct
  VARIANT 1: note = Q or E
  VARIANT 55: note = G or A
  VARIANT 63: note = S or A
  VARIANT 74: note = N or A
  VARIANT 75: note = S or A
  VARIANT 100: note = G or A
XVQLVESGGGVVQSGRSLRLSCAASGFMFSNFAMHWVRQAPGKGLEWVGVIWYDXSNKFYADXVKGRFTISRDXXKNTLNLQMSRLRAEDTAVYYCARDXREVWRYYHYGMDVWGQGTTVTVSS

SEQ ID NO: 212 - protein, 6 aa, synthetic construct
  VARIANT 4: note = G or A
WYDXSN

SEQ ID NO: 213 - protein, 17 aa, synthetic construct
  VARIANT 6: note = G or A
VIWYDXSNKFYADSVKG

SEQ ID NO: 214 - protein, 15 aa, synthetic construct
  VARIANT 2: note = G or A
DXREVWRYYHYGMDV

SEQ ID NO: 215 - protein, 107 aa, synthetic construct
  VARIANT 93: note = S or A
EIVLTQSPSTLSASVGDRVTITCRASQSISNWLAWYQQKPGKAPKLLIYKASSLESGVPSRFSGSGSGTEFTLTISSLQPDDFATYYCQQYNXYSYTFGQGTKVEIK

SEQ ID NO: 216 - protein, 117 aa, synthetic construct
QVQLVQSGAEVKKPGASVKVSCTASGYTFTGYFIHWVRQAPGQGLEWMGWINPHSGATNYAQKFQGRVTMTRDTSISTAYMELSSLRSDDTAVYYCARVSYYGLDVWGQGTTVTVSS

SEQ ID NO: 217 - protein, 7 aa, synthetic construct
GYTFTGY

SEQ ID NO: 218 - protein, 5 aa, synthetic construct
GYFIH

SEQ ID NO: 219 - protein, 6 aa, synthetic construct
NPHSGA

SEQ ID NO: 220 - protein, 17 aa, synthetic construct
WINPHSGATNYAQKFQG

SEQ ID NO: 221 - protein, 8 aa, synthetic construct
VSYYGLDV

SEQ ID NO: 222 - protein, 107 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSYGNPLTFGGGTKVEIK

SEQ ID NO: 223 - protein, 11 aa, synthetic construct
RASQSISSYLN

SEQ ID NO: 224 - protein, 7 aa, synthetic construct
AASSLQS

SEQ ID NO: 225 - protein, 9 aa, synthetic construct
QQSYGNPLT

SEQ ID NO: 226 - protein, 117 aa, synthetic construct
  VARIANT 1: note = Q or E
  VARIANT 48: note = M, V or I
  VARIANT 70: note = M,V, or I
XVQLVQSGAEVKKPGASVKVSCTASGYTFTGYFIHWVRQAPGQGLEWXGWINPHSGATNYAQKFQGRVTXTRDTSISTAYMELSSLRSDDTAVYYCARVSYYGLDVWGQGTTVTVSS

SEQ ID NO: 227 - protein, 117 aa, synthetic construct
QVQLQESGAEVKKPGASVKVSCKASGYTFTGYYLHWVRQAPGQGLEWMGWINPNSGDTNYAQKFQGRVTMTRDTSISTAYMELSRLRSDDTAVYYCARVSYYGLDVWGQGTTVTVSS

SEQ ID NO: 228 - protein, 5 aa, synthetic construct
GYYLH

SEQ ID NO: 229 - protein, 6 aa, synthetic construct
NPNSGD

SEQ ID NO: 230 - protein, 17 aa, synthetic construct
WINPNSGDTNYAQKFQG

SEQ ID NO: 231 - protein, 107 aa, synthetic construct
DIVLTQTPSSLSASVGDRVTITCWASQSISSYLNWYQQKPGKAPKLLIYAASSLQSGVPSRFSGSGSGTDFTLTISSLQPEDFATYYCQQSYSTPLTFGGGTKLEIK

SEQ ID NO: 232 - protein, 11 aa, synthetic construct
WASQSISSYLN

SEQ ID NO: 233 - protein, 9 aa, synthetic construct
QQSYSTPLT

SEQ ID NO: 234 - protein, 117 aa, synthetic construct
QVQLVQSGAEVKKPGASVKVSCKASGYTFTGYYLHWVRQAPGQGLEWMGWINPSSGDTNYAQKFQGRVTMTRDTSISTAYMELSRLRSDDTAVYYCARVSYYGLDVWGQGTTVTVSS

SEQ ID NO: 235 - protein, 6 aa, synthetic construct
NPSSGD

SEQ ID NO: 236 - protein, 17 aa, synthetic construct
WINPSSGDTNYAQKFQG

SEQ ID NO: 237 - protein, 107 aa, synthetic construct
EIVLTQSPSSLSASVGDRVTITCRASQSISSYLNWYQQKPGKAPKLLIYAASSLQSGVPSRFSGSGSGTDFTLTISSLQPEDFATYYCQQSYSNPLTFGGGTKVDIK

SEQ ID NO: 238 - protein, 9 aa, synthetic construct
QQSYSNPLT

SEQ ID NO: 239 - protein, 117 aa, synthetic construct
  VARIANT 1: note = Q or E
  VARIANT 5: note = Q or V
  VARIANT 6: note = E or Q
  VARIANT 48: note = M, V or I
  VARIANT 54: note = N or S
  VARIANT 55: note = S or A
  VARIANT 70: note = M, V or I
  VARIANT 81: note = M, V, or I
XVQLXXSGAEVKKPGASVKVSCKASGYTFTGYYLHWVRQAPGQGLEWXGWINPXXGDTNYAQKFQGRVTXTRDTSISTAYXELSRLRSDDTAVYYCARVSYYGLDVWGQGTTVTVSS

SEQ ID NO: 240 - protein, 6 aa, synthetic construct
  VARIANT 3: note = N or S
  VARIANT 4: note = S or A
NPXXGD

SEQ ID NO: 241 - protein, 17 aa, synthetic construct
  VARIANT 5: note = N or S
  VARIANT 6: note = S or A
WINPXXGDTNYAQKFQG

SEQ ID NO: 242 - protein, 122 aa, synthetic construct
  VARIANT 1: note = Q or E
  VARIANT 63: note = S or A
XVQLVESGGGLVQPGGSLRLSCAASGFTFSSYAMSWVRQAPGKGLEWVSEISGSGDRTSYADXVKGRFTISRDNSKNRLYLQMNRLRTEDTAVYYCAKDLGPSGWYGLFDYWGQGTLVTVSS

SEQ ID NO: 243 - protein, 117 aa, synthetic construct
QVQLVESGAEVKKPGASVKVSCKASGYTFTGYYLHWVRQAPGQGLEWMGWINPNNGDTNYAQKFQGRVTMTRDTSISTAYMELSRLRSDDTAVYYCARVSYYGLDVWGQGTTVTVSS

SEQ ID NO: 244 - protein, 6 aa, synthetic construct
NPNNGD

SEQ ID NO: 245 - protein, 17 aa, synthetic construct
WINPNNGDTNYAQKFQG

SEQ ID NO: 246 - protein, 107 aa, synthetic construct
DIRMTQSPSSLSASVGDRVTIICRASQSISSYLNWYQQKPGKAPKLLIYAASSLQSGVPSRFSGSGSGTDFTLTISSLQPEDFATYYCQQSYSNPLTFGGGTKVEIK

SEQ ID NO: 247 - protein, 117 aa, synthetic construct
EVQLVESGAEVKKPGASVKVSCKASGYTFTGYYLHWVRQAPGQGLEWMGWINPNNGDTNYAQKFQGRVTMTRDTSISTAYMELSRLRSDDTAVYYCARVSYYGLDVWGQGTTVTVSS

SEQ ID NO: 248 - protein, 117 aa, synthetic construct
  VARIANT 1: note = Q or E
XVQLVESGAEVKKPGASVKVSCKASGYTFTGYYLHWVRQAPGQGLEWMGWINPNNGDTNYAQKFQGRVTMTRDTSISTAYMELSRLRSDDTAVYYCARVSYYGLDVWGQGTTVTVSS

SEQ ID NO: 249 - protein, 122 aa, synthetic construct
QVQLVQSGGGLVQPGGSLRLSCAASGFTFSSYAMNWVRQAPGKGLEWVSGISGSGGSTKYAESVKGRFTISRDNSKNTLYLQMNSLRADDTAVYYCAKDQDINVWYGLFPYWGQGTLVTVSS

SEQ ID NO: 250 - protein, 5 aa, synthetic construct
SYAMN

SEQ ID NO: 251 - protein, 6 aa, synthetic construct
SGSGGS

SEQ ID NO: 252 - protein, 17 aa, synthetic construct
GISGSGGSTNYADSVKG

SEQ ID NO: 253 - protein, 17 aa, synthetic construct
GISGSGGSTKYAESVKG

SEQ ID NO: 254 - protein, 13 aa, synthetic construct
DQDINVWYGLFPY

SEQ ID NO: 255 - protein, 108 aa, synthetic construct
DIVLTQTPSSLSASVGDRVTITCRASQSISSWLAWYQQKPGKAPKLLIYKASSLESGVPSRFSGSGSGTEFTLTISSLQPDDFATYYCQQYNSYSPLTFGGGTKVDIK

SEQ ID NO: 256 - protein, 10 aa, synthetic construct
QQYNSYSPLT

SEQ ID NO: 257 - protein, 122 aa, synthetic construct
EVQLVQSGGGLVQPGGSLRLSCAASGFTFSSYAMNWVRQAPGKGLEWVSGISGSGGSTKYAESVKGRFTISRDNSKNTLYLQMNSLRADDTAVYYCAKDQDINVWYGLFPYWGQGTLVTVSS

SEQ ID NO: 258 - protein, 122 aa, synthetic construct
  VARIANT 1: note = Q or E
XVQLVQSGGGLVQPGGSLRLSCAASGFTFSSYAMNWVRQAPGKGLEWVSGISGSGGSTKYAESVKGRFTISRDNSKNTLYLQMNSLRADDTAVYYCAKDQDINVWYGLFPYWGQGTLVTVSS

SEQ ID NO: 259 - protein, 126 aa, synthetic construct
EVQLVESGGGVVQPGRSLRLSCAASGFTFSSSGMHWVRQAPGKGLEWVAVIWYDGSNKYYVDSVKGRFTISRDNSKNTLYLQMNSLRAEDTAVYYCARDGRQQLVQGSYYYGMDVWGQGTTVTVSS

SEQ ID NO: 260 - protein, 7 aa, synthetic construct
GFTFSSS

SEQ ID NO: 261 - protein, 5 aa, synthetic construct
SSGMH

SEQ ID NO: 262 - protein, 17 aa, synthetic construct
VIWYDGSNKYYVDSVKG

SEQ ID NO: 263 - protein, 107 aa, synthetic construct
EIVMTQSPSTLSASVGDRVTITCRASQSISSWLAWYQQKPGKAPKLLIYKASSLESGVPSRFSGSGSGTDFTLTISSLQPDDFATYYCQQYKSYSYTFGQGTKVEIK

SEQ ID NO: 264 - protein, 9 aa, synthetic construct
QQYKSYSYT

SEQ ID NO: 265 - protein, 17 aa, synthetic construct
DGRQQLVQGSYYYGMDV

SEQ ID NO: 266 - protein, 122 aa, synthetic construct
QVQLVQSGGGLVQRGGSLRLSCAASGFTFSSYAMSWVRQAPGKGLERVSGISGSGGNTYYADSVKGRFTISRDNSKNTLYLQMNSLRADDTAVYYCARDQDINVWYGLFAYWGQGTLVTVSS

SEQ ID NO: 267 - protein, 6 aa, synthetic construct
SGSGGN

SEQ ID NO: 268 - protein, 17 aa, synthetic construct
GISGSGGNTYYADSVKG

SEQ ID NO: 269 - protein, 13 aa, synthetic construct
DQDINVWYGLFAY

SEQ ID NO: 270 - protein, 108 aa, synthetic construct
DIVMTQTPSTLSASVGDRVTITCRASQSISSWLAWYQQKPGKAPKLLIYKASSLESGVPSRFSGSGSGTEFTLTISSLQPDDFATYYCQQYNSYFMYTFGQGTKVEIK

SEQ ID NO: 271 - protein, 10 aa, synthetic construct
QQYNSYFMYT

SEQ ID NO: 272 - protein, 122 aa, synthetic construct
EVQLVQSGGGLVQRGGSLRLSCAASGFTFSSYAMSWVRQAPGKGLERVSGISGSGGNTYYADSVKGRFTISRDNSKNTLYLQMNSLRADDTAVYYCARDQDINVWYGLFAYWGQGTLVTVSS

SEQ ID NO: 273 - protein, 122 aa, synthetic construct
  VARIANT 1: note = Q or E
XVQLVQSGGGLVQRGGSLRLSCAASGFTFSSYAMSWVRQAPGKGLERVSGISGSGGNTYYADSVKGRFTISRDNSKNTLYLQMNSLRADDTAVYYCARDQDINVWYGLFAYWGQGTLVTVSS

SEQ ID NO: 274 - protein, 119 aa, synthetic construct
QVQLQESGPGLVKPSETLSLTCAVSGYSISSGYYWGWIRQPPGKGLEWIGTIWHSGSTYDNPSLKSRVTISVDTSKNQFSLKLSSVTTADTAVYYCARADSSGWYEDYWGQGTLVTVSS

SEQ ID NO: 275 - protein, 8 aa, synthetic construct
GYSISSGY

SEQ ID NO: 276 - protein, 6 aa, synthetic construct
SGYYWG

SEQ ID NO: 277 - protein, 5 aa, synthetic construct
WHSGS

SEQ ID NO: 278 - protein, 16 aa, synthetic construct
TIWHSGSTYDNPSLKS

SEQ ID NO: 279 - protein, 10 aa, synthetic construct
ADSSGWYEDY

SEQ ID NO: 280 - protein, 107 aa, synthetic construct
DIVMTQTPSTLSASVGDRVTITCRASQSISSWLAWYQQKPGKAPKLLIYKASSLESGVPSRFSGSGSGTDFTLTISSLQPEDFATYYCQQSYSTPLTFGGGTKVEIK

SEQ ID NO: 281 - protein, 119 aa, synthetic construct
EVQLQESGPGLVKPSETLSLTCAVSGYSISSGYYWGWIRQPPGKGLEWIGTIWHSGSTYDNPSLKSRVTISVDTSKNQFSLKLSSVTTADTAVYYCARADSSGWYEDYWGQGTLVTVSS

SEQ ID NO: 282 - protein, 119 aa, synthetic construct
  VARIANT 1: note = Q or E
XVQLQESGPGLVKPSETLSLTCAVSGYSISSGYYWGWIRQPPGKGLEWIGTIWHSGSTYDNPSLKSRVTISVDTSKNQFSLKLSSVTTADTAVYYCARADSSGWYEDYWGQGTLVTVSS

SEQ ID NO: 283 - protein, 122 aa, synthetic construct
QVQLVQSGGGLVQPGGSLRLSCAASGFIFSSYAMSWVRQAPGRGLEWVSGISGSGGSTNYADSVKGRFTISRDNSKNTLYLQMNSLRADDTAVYFCAKDLDINVWYGLFDHWGQGTTVTVSS

SEQ ID NO: 284 - protein, 7 aa, synthetic construct
GFIFSSY

SEQ ID NO: 285 - protein, 13 aa, synthetic construct
DLDINVWYGLFDH

SEQ ID NO: 286 - protein, 108 aa, synthetic construct
DIQVTQSPSTLSASVGDRVTITCRASQSISSWLAWYQQKPGKAPKLLIYKASSLESGVPSRFSGSGSGTEFTLTISSLQPDDFATYYCQQYNSYSTWTFGQGTKLEIK

SEQ ID NO: 287 - protein, 122 aa, synthetic construct
EVQLVQSGGGLVQPGGSLRLSCAASGFIFSSYAMSWVRQAPGRGLEWVSGISGSGGSTNYADSVKGRFTISRDNSKNTLYLQMNSLRADDTAVYFCAKDLDINVWYGLFDHWGQGTTVTVSS

SEQ ID NO: 288 - protein, 122 aa, synthetic construct
EVQLVESGGGLVQPGGSLRLSCAASGFIFSSYAMNWVRQAPGRGLEWVSGISGSGGSTNYADSVKGRFTISRDNSKNTLYLQMNSLRADDTAVYFCARDQDINVWYGLFVYWGQGTLVTVSS

SEQ ID NO: 289 - protein, 122 aa, synthetic construct
  VARIANT 1: note = Q or E
XVQLVQSGGGLVQPGGSLRLSCAASGFIFSSYAMSWVRQAPGRGLEWVSGISGSGGSTNYADSVKGRFTISRDNSKNTLYLQMNSLRADDTAVYFCAKDLDINVWYGLFDHWGQGTTVTVSS

SEQ ID NO: 290 - protein, 13 aa, synthetic construct
DQDINVWYGLFVY

SEQ ID NO: 291 - protein, 108 aa, synthetic construct
DIRLTQSPSTLSASVGDRVTITCRASQSISSWLAWYQQKPGKAPKLLIYKASSLESGVPSRFSGSGSGTEFTLTISSMQPDDFATYYCQQYNSYSTWTFGQGTKVEIK

SEQ ID NO: 292 - protein, 118 aa, synthetic construct
QVQLVQSGGGLVQPGGSLRLSCAASGFTFSSYAMNWVRQAPGKGLEWVSVISGNGGYTHYADSVKGRFTISRDNSKNTLYLQMNSLRAEGTAVYYCAKDDWGGAFDIWGQGTTVTVSS

SEQ ID NO: 293 - protein, 6 aa, synthetic construct
SGNGGY

SEQ ID NO: 294 - protein, 17 aa, synthetic construct
VISGNGGYTHYADSVKG

SEQ ID NO: 295 - protein, 9 aa, synthetic construct
DDWGGAFDI

SEQ ID NO: 296 - protein, 107 aa, synthetic construct
DIVMTQTPSSLSASVGDRVTITCRASQGISNYLAWYQQKPGKVPKLLIYAASTLQSGVPSRFSGSGSGTDFTLTISSLQPEDVATYYCQKYNSAPRTFGQGTKVEIK

SEQ ID NO: 297 - protein, 9 aa, synthetic construct
QKYNSAPRT

SEQ ID NO: 298 - protein, 118 aa, synthetic construct
EVQLVQSGGGLVQPGGSLRLSCAASGFTFSSYAMNWVRQAPGKGLEWVSVISGNGGYTHYADSVKGRFTISRDNSKNTLYLQMNSLRAEGTAVYYCAKDDWGGAFDIWGQGTTVTVSS

SEQ ID NO: 299 - protein, 118 aa, synthetic construct
EVQLVQSGGGLVQPGGSVRLSCAASGFTFSSYAMNWVRQGPGKGLEWVSVISGNGGYTHYADSVKGRFTISRDNSKNTLYLQMNGLRADDTAVYYCAKDDWGGAFDIWGQGTMVTVSS

SEQ ID NO: 300 - protein, 107 aa, synthetic construct
DIRMTQSPSSLSASVGDRVTITCRASQGISNYLAWYQQKPGKVPKLLIYAASTLQSGVPSRFSGSGSGTDFTLTISSLQPEDVATYYCQKYNSAPRTFGQGTKVDIK

SEQ ID NO: 301 - protein, 118 aa, synthetic construct
  VARIANT 1: note = Q or E
XVQLVQSGGGLVQPGGSLRLSCAASGFTFSSYAMNWVRQAPGKGLEWVSVISGNGGYTHYADSVKGRFTISRDNSKNTLYLQMNSLRAEGTAVYYCAKDDWGGAFDIWGQGTTVTVSS

SEQ ID NO: 302 - protein, 107 aa, synthetic construct
  VARIANT 3: note = V or R
  VARIANT 7: note = S or T
  VARIANT 105: note = D or E
DIXMTQXPSSLSASVGDRVTITCRASQGISNYLAWYQQKPGKVPKLLIYAASTLQSGVPSRFSGSGSGTDFTLTISSLQPEDVATYYCQKYNSAPRTFGQGTKVXIK

SEQ ID NO: 303 - protein, 122 aa, synthetic construct
QVQLVESGGGVVQPGRSLRLSCAASGFTFSNYAMHWVRQAPGKGLEWVTLIWYDGSKKYYADSVKGRFTISRDNSKNTLYLQMNSLRAEDTAVYYCARDRAIPLIAPYFDYWGQGTLVTVSS

SEQ ID NO: 304 - protein, 7 aa, synthetic construct
GFTFSNY

SEQ ID NO: 305 - protein, 5 aa, synthetic construct
NYAMH

SEQ ID NO: 306 - protein, 6 aa, synthetic construct
WYDGSK

SEQ ID NO: 307 - protein, 17 aa, synthetic construct
LIWYDGSKKYYADSVKG

SEQ ID NO: 308 - protein, 13 aa, synthetic construct
DRAIPLIAPYFDY

SEQ ID NO: 309 - protein, 107 aa, synthetic construct
DIQLTQSPSFLSASVGDRVTITCRASQGISSYLAWYQQKPGKAPKLLIFPASTLQSGVPSRFSGSGSGTEFTLTISSLQPEDFATYYCQQLNFFPPTFGPGTKVDIK

SEQ ID NO: 310 - protein, 7 aa, synthetic construct
PASTLQS

SEQ ID NO: 311 - protein, 9 aa, synthetic construct
QQLNFFPPT

SEQ ID NO: 312 - protein, 122 aa, synthetic construct
EVQLVESGGGVVQPGRSLRLSCAASGFTFSNYAMHWVRQAPGKGLEWVTLIWYDGSKKYYADSVKGRFTISRDNSKNTLYLQMNSLRAEDTAVYYCARDRAIPLIAPYFDYWGQGTLVTVSS

SEQ ID NO: 313 - protein, 122 aa, synthetic construct
  VARIANT 1: note = Q or E
XVQLVESGGGVVQPGRSLRLSCAASGFTFSNYAMHWVRQAPGKGLEWVTLIWYDGSKKYYADSVKGRFTISRDNSKNTLYLQMNSLRAEDTAVYYCARDRAIPLIAPYFDYWGQGTLVTVSS

SEQ ID NO: 314 - protein, 115 aa, synthetic construct
QVQLVESGPGLVKPSETLSLTCTVSGGPIYSYYWSWIRQPPGKGLEWIGFIYSSGSTNYNPSLKSRVTISIDTSKSQFSLRLSSVTAADTAVYYCARGGDAFDIWGQGTMVTVSS

SEQ ID NO: 315 - protein, 7 aa, synthetic construct
GGPIYSY

SEQ ID NO: 316 - protein, 5 aa, synthetic construct
SYYWS

SEQ ID NO: 317 - protein, 5 aa, synthetic construct
YSSGS

SEQ ID NO: 318 - protein, 16 aa, synthetic construct
FIYSSGSTNYNPSLKS

SEQ ID NO: 319 - protein, 7 aa, synthetic construct
GGDAFDI

SEQ ID NO: 320 - protein, 115 aa, synthetic construct
EVQLVESGPGLVKPSETLSLTCTVSGGPIYSYYWSWIRQPPGKGLEWIGFIYSSGSTNYNPSLKSRVTISIDTSKSQFSLRLSSVTAADTAVYYCARGGDAFDIWGQGTMVTVSS

SEQ ID NO: 321 - protein, 115 aa, synthetic construct
  VARIANT 1: note = Q or E
XVQLVESGPGLVKPSETLSLTCTVSGGPIYSYYWSWIRQPPGKGLEWIGFIYSSGSTNYNPSLKSRVTISIDTSKSQFSLRLSSVTAADTAVYYCARGGDAFDIWGQGTMVTVSS

SEQ ID NO: 322 - protein, 115 aa, synthetic construct
QVQLQESGPGLVKPSETLSLTCTVSGGSLSSYYWSWIRQPPGKGLEWLGYIYSSGSVNYNPSLKSRVTMSIDTSQNQFSLKLSSVTAADTAVYYCARDADYFDYWGQGTLVTVSS

SEQ ID NO: 323 - protein, 7 aa, synthetic construct
GGSLSSY

SEQ ID NO: 324 - protein, 16 aa, synthetic construct
YIYSSGSVNYNPSLKS

SEQ ID NO: 325 - protein, 7 aa, synthetic construct
DADYFDY

SEQ ID NO: 326 - protein, 107 aa, synthetic construct
DIQLTQSPSFLSASVGDRVTITCRASQGTSSYLAWYQQKPGKAPKLLIYAVSTLQSGVPSRFSGSGSGTEFTLTISSLQPTDFATYYCQQVIRYPPTFGQGTKVEVK

SEQ ID NO: 327 - protein, 11 aa, synthetic construct
RASQGTSSYLA

SEQ ID NO: 328 - protein, 7 aa, synthetic construct
AVSTLQS

SEQ ID NO: 329 - protein, 9 aa, synthetic construct
QQVIRYPPT

SEQ ID NO: 330 - protein, 115 aa, synthetic construct
EVQLQESGPGLVKPSETLSLTCTVSGGSLSSYYWSWIRQPPGKGLEWLGYIYSSGSVNYNPSLKSRVTMSIDTSQNQFSLKLSSVTAADTAVYYCARDADYFDYWGQGTLVTVSS

SEQ ID NO: 331 - protein, 115 aa, synthetic construct
  VARIANT 1: note = Q or E
XVQLQESGPGLVKPSETLSLTCTVSGGSLSSYYWSWIRQPPGKGLEWLGYIYSSGSVNYNPSLKSRVTMSIDTSQNQFSLKLSSVTAADTAVYYCARDADYFDYWGQGTLVTVSS

SEQ ID NO: 332 - protein, 126 aa, synthetic construct
EVQLVESGGDVVQPGRSLRLSCAASGFTLSSYGMHWVRQAPGKGLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 333 - protein, 126 aa, synthetic construct
QVQLVESGGDVVQPGRSLRLSCAASGFTLSSYGMHWVRQAPGKGLEWVAAMWYDGSNNYYEDTVKGRFTISRDNSKNTLYLQMNSLRAEDTAVYYCARERVSRHFDWHYYYGMDVWGQGTTVTVSS

SEQ ID NO: 334 - protein, 126 aa, synthetic construct
QVQLVESGGDVVQPGRSLRLSCAASGFTLSSYGMHWVRQAPGKGLEWVAAMWYDGSNNYYEDSVKGRFTISRDQSKNTLYLQMNSLRAEDTAVYYCARERVSRHFDWHYYYGMDVWGQGTTVTVSS

SEQ ID NO: 335 - protein, 126 aa, synthetic construct
QVQLVESGGDVVQPGRSLRLSCAASGFTLSSYGMHWVRQAPGKGLEWVAAMWYDGSNNYYEDSVKGRFTISRDNSKQTLYLQMNSLRAEDTAVYYCARERVSRHFDWHYYYGMDVWGQGTTVTVSS

SEQ ID NO: 336 - protein, 126 aa, synthetic construct
QVQLVESGGDVVQPGRSLRLSCAASGFTLSSYGMHWVRQAPGKGLEWVAAMWYDGSNNYYEDSVKGRFTISRDNSKNTLYLQMQSLRAEDTAVYYCARERVSRHFDWHYYYGMDVWGQGTTVTVSS

SEQ ID NO: 337 - protein, 11 aa, synthetic construct
GSSGSYFDLDI

SEQ ID NO: 338 - protein, 120 aa, synthetic construct
EVQLQESGPGLVKPSGTLSLTCAVSGGSISSSDWWTWVRQPPGKGLEWIGEIYHSGSTNYNPSLKSRVTISVDKSKNQFSLNLNSVTAADTAVYYCARGYSGSYFDLDIWGQGTTVTVSS

SEQ ID NO: 339 - protein, 120 aa, synthetic construct
QVQLQESGPGLVKPSGTLSLTCAVSGGSISSSDWWTWVRQPPGKGLEWIGEIYHSGSTNYNPSLKSRVTISVDKSKNQFSLNLNAVTAADTAVYYCARGYSGSYFDLDIWGQGTTVTVSS

SEQ ID NO: 340 - protein, 107 aa, synthetic construct
EIVMTQSPSSLSASVGDRVTITCRASQGISNYLAWYQQKPGKVPKLLIYAASTLQSGVPSRFSGSGSGTEFTLTISSLQPEDFATYYCQQLNAYPLTFGGGTKVEIK

SEQ ID NO: 341 - protein, 9 aa, synthetic construct
QQLNAYPLT

SEQ ID NO: 342 - protein, 120 aa, synthetic construct
EVQLVQSGPGLVKPSGTLSLTCAVSGGSISSSDWWTWVRQPPGKGLEWIGEIYHSGSTNYNPSLKSRVTISVDKSKNQFSLKLNSVTAADTAVYYCARGSSGSYFDLDIWGQGTTVTVSS

SEQ ID NO: 343 - protein, 120 aa, synthetic construct
QVQLVQSGPGLVKPSGTLSLTCAVSGGSISSSDWWTWVRQPPGKGLEWIGEIYHSGSTNYNPSLKSRVTISVDKSKNQFSLKLNAVTAADTAVYYCARGSSGSYFDLDIWGQGTTVTVSS

SEQ ID NO: 344 - protein, 107 aa, synthetic construct
DIVMTQSPSTLSASVGDRVTITCRASQGISSYLAWYQQKPGKAPKLLIYAASTLQSGVPSRFSGSGSGTEFTLTISSLQPEDFATYYCQQLNAYPLTFGGGTKVEIK

SEQ ID NO: 345 - protein, 120 aa, synthetic construct
EVQLQESGPGLVKPSGTLSLTCAVSGGSISSSKWWSWVRQSPGKGLEWIGEIFHSGSINHNTSFKSRVTISVDKSKNQFSLKLSSVTAADTAVYYCARGGSGSYDAFDIWGQGTMVTVSS

SEQ ID NO: 346 - protein, 120 aa, synthetic construct
QVQLQESGPGLVKPSGTLSLTCAVSGGSISSSKWWSWVRQSPGKGLEWIGEIFHSGSINHQTSFKSRVTISVDKSKNQFSLKLSSVTAADTAVYYCARGGSGSYDAFDIWGQGTMVTVSS

SEQ ID NO: 347 - protein, 16 aa, synthetic construct
EIFHSGSINHQTSFKS

SEQ ID NO: 348 - protein, 120 aa, synthetic construct
QVQLQESGPGLVKPSGTLSLTCAVSGGSISSSKWWSWVRQSPGKGLEWIGEIFHSGSINHNTAFKSRVTISVDKSKNQFSLKLSSVTAADTAVYYCARGGSGSYDAFDIWGQGTMVTVSS

SEQ ID NO: 349 - protein, 16 aa, synthetic construct
EIFHSGSINHNTAFKS

SEQ ID NO: 350 - protein, 122 aa, synthetic construct
EVQLQESGPGLVKPSGTLSLTCAVSGGSISSSDWWTWVRQPPGKGLEWIGEIYHSGSTNYNPSLKSRVTISVDKSKNQFSLNLNSVTAADTAVYYCARGYSGSYFDLDIWGQGTTVTVSSGS

SEQ ID NO: 351 - protein, 122 aa, synthetic construct
QVQLQESGPGLVKPSGTLSLTCAVSGGSISSSDWWTWVRQPPGKGLEWIGEIYHSGSTNYNPSLKSRVTISVDKSKNQFSLNLNAVTAADTAVYYCARGYSGSYFDLDIWGQGTTVTVSSGS

SEQ ID NO: 352 - protein, 122 aa, synthetic construct
EVQLVESGGGLVQPGGSLRLSCAASGFTFSSYAMSWVRQAPGKGLEWVSEISGSGDRTSYADSVKGRFTISRDNSKNRLYLQMNRLRTEDTAVYYCAKDLGPSGWYGLFDYWGQGTLVTVSS

SEQ ID NO: 353 - protein, 122 aa, synthetic construct
QVQLVESGGGLVQPGGSLRLSCAASGFTFSSYAMSWVRQAPGKGLEWVSEISGSGDRTSYADAVKGRFTISRDNSKNRLYLQMNRLRTEDTAVYYCAKDLGPSGWYGLFDYWGQGTLVTVSS

SEQ ID NO: 354 - protein, 122 aa, synthetic construct
QVQLVESGGGLVQPGGSLRLSCAASGFTFSSYAMSWVRQAPGKGLEWVSEISGSGDRTSYADSVKGRFTISRDASKNRLYLQMNRLRTEDTAVYYCAKDLGPSGWYGLFDYWGQGTLVTVSS

SEQ ID NO: 355 - protein, 122 aa, synthetic construct
QVQLVESGGGLVQPGGSLRLSCAASGFTFSSYAMSWVRQAPGKGLEWVSEISGSGDRTSYADSVKGRFTISRDNAKNRLYLQMNRLRTEDTAVYYCAKDLGPSGWYGLFDYWGQGTLVTVSS

SEQ ID NO: 356 - protein, 120 aa, synthetic construct
EVQLVQSGAEVVKPGATVKLSCKASDFNIKDAYMHWVQQAPGKGLEWIGWIDPENGDTEYAPKFQGRATITADTSTDTAYLELSSLRSEDTAVYYCNVITTVVNYAMDYWGQGTLVTVSS

SEQ ID NO: 357 - protein, 124 aa, synthetic construct
EVQLVESGGGVVQSGRSLRLSCAASGFMFSNFAMHWVRQAPGKGLEWVGVIWYDGSNKFYADSVKGRFTISRDNSKNTLNLQMSRLRAEDTAVYYCARDGREVWRYYHYGMDVWGQGTTVTVSS

SEQ ID NO: 358 - protein, 124 aa, synthetic construct
QVQLVESGGGVVQSGRSLRLSCAASGFMFSNFAMHWVRQAPGKGLEWVGVIWYDASNKFYADSVKGRFTISRDNSKNTLNLQMSRLRAEDTAVYYCARDGREVWRYYHYGMDVWGQGTTVTVSS

SEQ ID NO: 359 - protein, 6 aa, synthetic construct
WYDASN

SEQ ID NO: 360 - protein, 17 aa, synthetic construct
VIWYDASNKFYADSVKG

SEQ ID NO: 361 - protein, 124 aa, synthetic construct
QVQLVESGGGVVQSGRSLRLSCAASGFMFSNFAMHWVRQAPGKGLEWVGVIWYDGSNKFYADAVKGRFTISRDNSKNTLNLQMSRLRAEDTAVYYCARDGREVWRYYHYGMDVWGQGTTVTVSS

SEQ ID NO: 362 - protein, 17 aa, synthetic construct
VIWYDGSNKFYADAVKG

SEQ ID NO: 363 - protein, 124 aa, synthetic construct
QVQLVESGGGVVQSGRSLRLSCAASGFMFSNFAMHWVRQAPGKGLEWVGVIWYDGSNKFYADSVKGRFTISRDASKNTLNLQMSRLRAEDTAVYYCARDGREVWRYYHYGMDVWGQGTTVTVSS

SEQ ID NO: 364 - protein, 124 aa, synthetic construct
QVQLVESGGGVVQSGRSLRLSCAASGFMFSNFAMHWVRQAPGKGLEWVGVIWYDGSNKFYADSVKGRFTISRDNAKNTLNLQMSRLRAEDTAVYYCARDGREVWRYYHYGMDVWGQGTTVTVSS

SEQ ID NO: 365 - protein, 124 aa, synthetic construct
QVQLVESGGGVVQSGRSLRLSCAASGFMFSNFAMHWVRQAPGKGLEWVGVIWYDGSNKFYADSVKGRFTISRDNSKNTLNLQMSRLRAEDTAVYYCARDAREVWRYYHYGMDVWGQGTTVTVSS

SEQ ID NO: 366 - protein, 15 aa, synthetic construct
DAREVWRYYHYGMDV

SEQ ID NO: 367 - protein, 117 aa, synthetic construct
EVQLVQSGAEVKKPGASVKVSCTASGYTFTGYFIHWVRQAPGQGLEWMGWINPHSGATNYAQKFQGRVTMTRDTSISTAYMELSSLRSDDTAVYYCARVSYYGLDVWGQGTTVTVSS

SEQ ID NO: 368 - protein, 117 aa, synthetic construct
QVQLVQSGAEVKKPGASVKVSCTASGYTFTGYFIHWVRQAPGQGLEWIGWINPHSGATNYAQKFQGRVTMTRDTSISTAYMELSSLRSDDTAVYYCARVSYYGLDVWGQGTTVTVSS

SEQ ID NO: 369 - protein, 117 aa, synthetic construct
QVQLVQSGAEVKKPGASVKVSCTASGYTFTGYFIHWVRQAPGQGLEWVGWINPHSGATNYAQKFQGRVTMTRDTSISTAYMELSSLRSDDTAVYYCARVSYYGLDVWGQGTTVTVSS

SEQ ID NO: 370 - protein, 117 aa, synthetic construct
QVQLVQSGAEVKKPGASVKVSCTASGYTFTGYFIHWVRQAPGQGLEWMGWINPHSGATNYAQKFQGRVTITRDTSISTAYMELSSLRSDDTAVYYCARVSYYGLDVWGQGTTVTVSS

SEQ ID NO: 371 - protein, 117 aa, synthetic construct
QVQLVQSGAEVKKPGASVKVSCTASGYTFTGYFIHWVRQAPGQGLEWMGWINPHSGATNYAQKFQGRVTVTRDTSISTAYMELSSLRSDDTAVYYCARVSYYGLDVWGQGTTVTVSS

SEQ ID NO: 372 - protein, 117 aa, synthetic construct
EVQLQESGAEVKKPGASVKVSCKASGYTFTGYYLHWVRQAPGQGLEWMGWINPNSGDTNYAQKFQGRVTMTRDTSISTAYMELSRLRSDDTAVYYCARVSYYGLDVWGQGTTVTVSS

SEQ ID NO: 373 - protein, 117 aa, synthetic construct
QVQLQESGAEVKKPGASVKVSCKASGYTFTGYYLHWVRQAPGQGLEWMGWINPSSGDTNYAQKFQGRVTMTRDTSISTAYMELSRLRSDDTAVYYCARVSYYGLDVWGQGTTVTVSS

SEQ ID NO: 374 - protein, 117 aa, synthetic construct
QVQLQESGAEVKKPGASVKVSCKASGYTFTGYYLHWVRQAPGQGLEWMGWINPNAGDTNYAQKFQGRVTMTRDTSISTAYMELSRLRSDDTAVYYCARVSYYGLDVWGQGTTVTVSS

SEQ ID NO: 375 - protein, 6 aa, synthetic construct
NPNAGD

SEQ ID NO: 376 - protein, 17 aa, synthetic construct
WINPNAGDTNYAQKFQG

SEQ ID NO: 377 - protein, 124 aa, synthetic construct
QVQLVESGGGVVQSGRSLRLSCAASGFMFSNFAMHWVRQAPGKGLEWVGVIWYDGSNKFYADSVKGRFTISRDNSKNTLNLQMSRLRAEDTAVYYCARDGREVWRYYHYGMDVWGQGTTVTVSS

SEQ ID NO: 378 - protein, 117 aa, synthetic construct
QVQLQESGAEVKKPGASVKVSCKASGYTFTGYYLHWVRQAPGQGLEWIGWINPNSGDTNYAQKFQGRVTMTRDTSISTAYMELSRLRSDDTAVYYCARVSYYGLDVWGQGTTVTVSS

SEQ ID NO: 379 - protein, 117 aa, synthetic construct
QVQLQESGAEVKKPGASVKVSCKASGYTFTGYYLHWVRQAPGQGLEWVGWINPNSGDTNYAQKFQGRVTMTRDTSISTAYMELSRLRSDDTAVYYCARVSYYGLDVWGQGTTVTVSS

SEQ ID NO: 380 - protein, 117 aa, synthetic construct
QVQLQESGAEVKKPGASVKVSCKASGYTFTGYYLHWVRQAPGQGLEWMGWINPNSGDTNYAQKFQGRVTITRDTSISTAYMELSRLRSDDTAVYYCARVSYYGLDVWGQGTTVTVSS

SEQ ID NO: 381 - protein, 117 aa, synthetic construct
QVQLQESGAEVKKPGASVKVSCKASGYTFTGYYLHWVRQAPGQGLEWMGWINPNSGDTNYAQKFQGRVTVTRDTSISTAYMELSRLRSDDTAVYYCARVSYYGLDVWGQGTTVTVSS

SEQ ID NO: 382 - protein, 117 aa, synthetic construct
QVQLQESGAEVKKPGASVKVSCKASGYTFTGYYLHWVRQAPGQGLEWMGWINPNSGDTNYAQKFQGRVTMTRDTSISTAYIELSRLRSDDTAVYYCARVSYYGLDVWGQGTTVTVSS

SEQ ID NO: 383 - protein, 117 aa, synthetic construct
QVQLQESGAEVKKPGASVKVSCKASGYTFTGYYLHWVRQAPGQGLEWMGWINPNSGDTNYAQKFQGRVTMTRDTSISTAYVELSRLRSDDTAVYYCARVSYYGLDVWGQGTTVTVSS

SEQ ID NO: 384 - protein, 117 aa, synthetic construct
EVQLVQSGAEVKKPGASVKVSCKASGYTFTGYYLHWVRQAPGQGLEWMGWINPSSGDTNYAQKFQGRVTMTRDTSISTAYMELSRLRSDDTAVYYCARVSYYGLDVWGQGTTVTVSS

SEQ ID NO: 385 - protein, 117 aa, synthetic construct
QVQLVQSGAEVKKPGASVKVSCKASGYTFTGYYLHWVRQAPGQGLEWIGWINPSSGDTNYAQKFQGRVTMTRDTSISTAYMELSRLRSDDTAVYYCARVSYYGLDVWGQGTTVTVSS

SEQ ID NO: 386 - protein, 117 aa, synthetic construct
QVQLVQSGAEVKKPGASVKVSCKASGYTFTGYYLHWVRQAPGQGLEWVGWINPSSGDTNYAQKFQGRVTMTRDTSISTAYMELSRLRSDDTAVYYCARVSYYGLDVWGQGTTVTVSS

SEQ ID NO: 387 - protein, 117 aa, synthetic construct
QVQLVQSGAEVKKPGASVKVSCKASGYTFTGYYLHWVRQAPGQGLEWMGWINPSSGDTNYAQKFQGRVTITRDTSISTAYMELSRLRSDDTAVYYCARVSYYGLDVWGQGTTVTVSS

SEQ ID NO: 388 - protein, 117 aa, synthetic construct
QVQLVQSGAEVKKPGASVKVSCKASGYTFTGYYLHWVRQAPGQGLEWMGWINPSSGDTNYAQKFQGRVTVTRDTSISTAYMELSRLRSDDTAVYYCARVSYYGLDVWGQGTTVTVSS

SEQ ID NO: 389 - protein, 117 aa, synthetic construct
QVQLVQSGAEVKKPGASVKVSCKASGYTFTGYYLHWVRQAPGQGLEWMGWINPSSGDTNYAQKFQGRVTMTRDTSISTAYIELSRLRSDDTAVYYCARVSYYGLDVWGQGTTVTVSS

SEQ ID NO: 390 - protein, 117 aa, synthetic construct
QVQLVQSGAEVKKPGASVKVSCKASGYTFTGYYLHWVRQAPGQGLEWMGWINPSSGDTNYAQKFQGRVTMTRDTSISTAYVELSRLRSDDTAVYYCARVSYYGLDVWGQGTTVTVSS

SEQ ID NO: 391 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 392 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 393 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 394 - protein, 9 aa, synthetic construct
GSFSGYYWS

SEQ ID NO: 395 - protein, 16 aa, synthetic construct
EIDHSGSTNYNPSLKS

SEQ ID NO: 396 - protein, 11 aa, synthetic construct
ARARGPWSFDP

SEQ ID NO: 397 - protein, 117 aa, synthetic construct; identical to SEQ ID NO: 392

SEQ ID NO: 398 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 399 - protein, 117 aa, synthetic construct; identical to SEQ ID NO: 392

SEQ ID NO: 400 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 401 - protein, 117 aa, synthetic construct; identical to SEQ ID NO: 392

SEQ ID NO: 402 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 403 - protein, 117 aa, synthetic construct; identical to SEQ ID NO: 392

SEQ ID NO: 404 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 405 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 406 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 407 - protein, 117 aa, synthetic construct; identical to SEQ ID NO: 392

SEQ ID NO: 408 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 409 - protein, 117 aa, synthetic construct; identical to SEQ ID NO: 392

SEQ ID NO: 410 - protein, 107 aa, synthetic construct
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 411 - protein, 117 aa, synthetic construct; identical to SEQ ID NO: 392

SEQ ID NO: 412 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 413 - protein, 117 aa, synthetic construct; identical to SEQ ID NO: 392

SEQ ID NO: 414 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 415 - protein, 117 aa, synthetic construct; identical to SEQ ID NO: 392

SEQ ID NO: 416 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 417 - protein, 117 aa, synthetic construct; identical to SEQ ID NO: 392

SEQ ID NO: 418 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 419 - protein, 117 aa, synthetic construct; identical to SEQ ID NO: 392

SEQ ID NO: 420 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 421 - protein, 117 aa, synthetic construct; identical to SEQ ID NO: 392

SEQ ID NO: 422 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 423 - protein, 117 aa, synthetic construct; identical to SEQ ID NO: 392

SEQ ID NO: 424 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 425 - protein, 117 aa, synthetic construct; identical to SEQ ID NO: 392

SEQ ID NO: 426 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 427 - protein, 117 aa, synthetic construct; identical to SEQ ID NO: 392

SEQ ID NO: 428 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 429 - protein, 117 aa, synthetic construct; identical to SEQ ID NO: 392

SEQ ID NO: 430 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 431 - protein, 117 aa, synthetic construct; identical to SEQ ID NO: 392

SEQ ID NO: 432 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 433 - protein, 117 aa, synthetic construct; identical to SEQ ID NO: 392

SEQ ID NO: 434 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 435 - protein, 125 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 436 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 437 - protein, 9 aa, synthetic construct
GTFSSYAIS

SEQ ID NO: 438 - protein, 5 aa, synthetic construct
SYAIS

SEQ ID NO: 439 - protein, 17 aa, synthetic construct
GIIPIFGTANYAQKFQG

SEQ ID NO: 440 - protein, 17 aa, synthetic construct
KSSQSVLYSSNNKNYLA

SEQ ID NO: 441 - protein, 7 aa, synthetic construct
WASTRES

SEQ ID NO: 442 - protein, 18 aa, synthetic construct
ARGDSSIRHAYYYYGMDV

SEQ ID NO: 443 - protein, 16 aa, synthetic construct
GDSSIRHAYYYYGMDV

SEQ ID NO: 444 - protein, 9 aa, synthetic construct
QQYYSTPIT

SEQ ID NO: 445 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 446 - protein, 11 aa, synthetic construct
GSISSSSYYWG

SEQ ID NO: 447 - protein, 7 aa, synthetic construct
SSSYYWG

SEQ ID NO: 448 - protein, 16 aa, synthetic construct
SIYYSGSTYYNPSLKS

SEQ ID NO: 449 - protein, 13 aa, synthetic construct
ARGSDRFHPYFDY

SEQ ID NO: 450 - protein, 11 aa, synthetic construct
GSDRFHPYFDY

SEQ ID NO: 451 - protein, 11 aa, synthetic construct
RASQSVSRYLA

SEQ ID NO: 452 - protein, 7 aa, synthetic construct
DASNRAT

SEQ ID NO: 453 - protein, 9 aa, synthetic construct
QQFDTWPPT

SEQ ID NO: 454 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 455 - protein, 117 aa, synthetic construct; identical to SEQ ID NO: 392

SEQ ID NO: 456 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 457 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 458 - protein, 113 aa, synthetic construct [sequence omitted]

SEQ ID NO: 459 - protein, 17 aa, synthetic construct
GIIPIFGTANYAQKFQG

SEQ ID NO: 460 - protein, 19 aa, synthetic construct
ARRGRKASGSFYYYYGMDV

SEQ ID NO: 461 - protein, 17 aa, synthetic construct
ESSQSLLNSGNQKNYLT

SEQ ID NO: 462 - protein, 9 aa, synthetic construct
QNDYSYPYT

SEQ ID NO: 463 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 464 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 465 - protein, 9 aa, synthetic construct
YTFTSYYMH

SEQ ID NO: 466 - protein, 5 aa, synthetic construct
SYYMH

SEQ ID NO: 467 - protein, 17 aa, synthetic construct
IINPSGGSTSYAQKFQG

SEQ ID NO: 468 - protein, 19 aa, synthetic construct
ARGAPNYGDTTHDYYYMDV

SEQ ID NO: 469 - protein, 17 aa, synthetic construct
GAPNYGDTTHDYYYMDV

SEQ ID NO: 470 - protein, 11 aa, synthetic construct
RASQSVSSNLA

SEQ ID NO: 471 - protein, 126 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 472 - protein, 9 aa, synthetic construct
QQYDDWPFT

SEQ ID NO: 473 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 474 - protein, 107 aa, synthetic construct
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 475 - protein, 9 aa, synthetic construct
YTFTGYYMH

SEQ ID NO: 476 - protein, 5 aa, synthetic construct
GYYMH

SEQ ID NO: 477 - protein, 17 aa, synthetic construct
WINPNSGGTNYAQKFQG

SEQ ID NO: 478 - protein, 17 aa, synthetic construct
ARDTGEYYDTDDHGMDV

SEQ ID NO: 479 - protein, 15 aa, synthetic construct
DTGEYYDTDDHGMDV

SEQ ID NO: 480 - protein, 11 aa, synthetic construct
RASQSVSSNLA

SEQ ID NO: 481 - protein, 9 aa, synthetic construct
QQDDYWPPT

SEQ ID NO: 482 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 483 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 484 - protein, 9 aa, synthetic construct
FTFSSYAMS

SEQ ID NO: 485 - protein, 112 aa, synthetic construct
EIVMTQSPLSLPVTPGEPASISCRSSQSLLHSNAYNYLDWYLQKPGQSPQLLISLGSIRASGVPDRFSGSGSGTDFTLTISRVEAEDVGFYYCMQALQTPRTFGCGTKVDIT

SEQ ID NO: 486 - protein, 17 aa, synthetic construct
AISGSGGSTYYADSVKG

SEQ ID NO: 487 - protein, 14 aa, synthetic construct
AKDGGYYDSGAGDY

SEQ ID NO: 488 - protein, 12 aa, synthetic construct
DGGYYDSGAGDY

SEQ ID NO: 489 - protein, 11 aa, synthetic construct
RASQGIDSWLA

SEQ ID NO: 490 - protein, 258 aa, synthetic construct
EIVMTQSPLSLPVTPGEPASISCRSSQSLLHSQGYNYLDWYLQKPGQSPQLLISLGSIRASGVPDRFSGSGSGTDFTLTISRVEAEDVGFYYCMQALQTPRTFGCGTKVDITGGGGSGGGGSGGGGSGGGGSEVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 491 - protein, 9 aa, synthetic construct
QQGVSYPRT

SEQ ID NO: 492 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 493 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 494 - protein, 9 aa, synthetic construct
FTFSSYSMN

SEQ ID NO: 495 - protein, 5 aa, synthetic construct
SYSMN

SEQ ID NO: 496 - protein, 17 aa, synthetic construct
SISSSSSYIYYADSVKG

SEQ ID NO: 497 - protein, 15 aa, synthetic construct
ARGAPMGAAAGWFDP

SEQ ID NO: 498 - protein, 13 aa, synthetic construct
GAPMGAAAGWFDP

SEQ ID NO: 499 - protein, 9 aa, synthetic construct
QQGVSFPRT

SEQ ID NO: 500 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 501 - protein, 125 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 502 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 503 - protein, 17 aa, synthetic construct
IINPSGGSTSYAQKFQG

SEQ ID NO: 504 - protein, 18 aa, synthetic construct
AREGAGFAYGMDYYYMDV

SEQ ID NO: 505 - protein, 16 aa, synthetic construct
EGAGFAYGMDYYYMDV

SEQ ID NO: 506 - protein, 11 aa, synthetic construct
RASQSVSSYLA

SEQ ID NO: 507 - protein, 9 aa, synthetic construct
QQSDNWPFT

SEQ ID NO: 508 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 509 - protein, 15 aa, synthetic construct
ARGAPIGAAAGWFDP

SEQ ID NO: 510 - protein, 13 aa, synthetic construct
GAPIGAAAGWFDP

SEQ ID NO: 511 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 512 - protein, 15 aa, synthetic construct
ARGAPQGAAAGWFDP

SEQ ID NO: 513 - protein, 13 aa, synthetic construct
GAPQGAAAGWFDP

SEQ ID NO: 514 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 515 - protein, 15 aa, synthetic construct
ARGAPLGAAAGWFDP

SEQ ID NO: 516 - protein, 13 aa, synthetic construct
GAPLGAAAGWFDP

SEQ ID NO: 517 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 518 - protein, 15 aa, synthetic construct
ARGAPFGAAAGWFDP

SEQ ID NO: 519 - protein, 13 aa, synthetic construct
GAPFGAAAGWFDP

SEQ ID NO: 520 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 521 - protein, 15 aa, synthetic construct
ARGAPVGAAAGWFDP

SEQ ID NO: 522 - protein, 13 aa, synthetic construct
GAPVGAAAGWFDP

SEQ ID NO: 523 - protein, 122 aa, synthetic construct
  VARIANT 102: note = M, L, I, V, Q, or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 524 - protein, 15 aa, synthetic construct
  VARIANT 6: note = M, L, I, V, Q, or F
ARGAPXGAAAGWFDP

SEQ ID NO: 525 - protein, 13 aa, synthetic construct
  VARIANT 4: note = M, L, I, V, Q, or F
GAPXGAAAGWFDP

SEQ ID NO: 526 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 527 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 528 - protein, 115 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 529 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 530 - protein, 11 aa, synthetic construct
RASQGISSWLA

SEQ ID NO: 531 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 532 - protein, 20 aa, synthetic construct
GGGGSGGGGSGGGGSGGGGS

SEQ ID NO: 533 - protein, 40 aa, synthetic construct
  VARIANT 1..40: note = This sequence may encompass 1-20 GS repeating units
GSGSGSGSGSGSGSGSGSGSGSGSGSGSGSGSGSGSGSGS

SEQ ID NO: 534 - protein, 60 aa, synthetic construct
  VARIANT 1..60: note = This sequence may encompass 1-20 GGS repeating units
GGSGGSGGSGGSGGSGGSGGSGGSGGSGGSGGSGGSGGSGGSGGSGGSGGSGGSGGSGGS

SEQ ID NO: 535 - protein, 80 aa, synthetic construct
  VARIANT 1..80: note = This sequence may encompass 1-20 GGGS repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 536 - protein, 80 aa, synthetic construct
  VARIANT 1..80: note = This sequence may encompass 1-20 GGSG repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 537 - protein, 100 aa, synthetic construct
  VARIANT 1..100: note = This sequence may encompass 1-20 GGSGG repeating units
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 538 - protein, 100 aa, synthetic construct
  VARIANT 1..100: note = This sequence may encompass 1-20 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 539 - protein, 20 aa, synthetic construct
GSGSGSGSGSGSGSGSGSGS

SEQ ID NO: 540 - protein, 30 aa, synthetic construct
GGSGGSGGSGGSGGSGGSGGSGGSGGSGGS

SEQ ID NO: 541 - protein, 40 aa, synthetic construct
GGGSGGGSGGGSGGGSGGGSGGGSGGGSGGGSGGGSGGGS

SEQ ID NO: 542 - protein, 40 aa, synthetic construct
GGSGGGSGGGSGGGSGGGSGGGSGGGSGGGSGGGSGGGSG

SEQ ID NO: 543 - protein, 50 aa, synthetic construct
GGSGGGGSGGGGSGGGGSGGGGSGGGGSGGGGSGGGGSGGGGSGGGGSGG

SEQ ID NO: 544 - protein, 50 aa, synthetic construct
GGGGSGGGGSGGGGSGGGGSGGGGSGGGGSGGGGSGGGGSGGGGSGGGGS

SEQ ID NO: 545 - protein, 15 aa, synthetic construct
GGGGSGGGGSGGGGS

SEQ ID NO: 546 - protein, 100 aa, synthetic construct; identical to SEQ ID NO: 538

SEQ ID NO: 547 - protein, 100 aa, synthetic construct; identical to SEQ ID NO: 537

SEQ ID NO: 548 - protein, 476 aa, synthetic construct
DIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIKGGGGSGGGGSGGGGSGGGGSQVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 549 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 550 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 551 - protein, 476 aa, synthetic construct
EVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSSGGGGSGGGGSGGGGSGGGGSDIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 552 - protein, 477 aa, synthetic construct
QVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSGGGGSGGGGSGGGGSGGGGSDIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 553 - protein, 477 aa, synthetic construct
EVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSGGGGSGGGGSGGGGSGGGGSDIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 554 - protein, 477 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGGGGSGGGGSGGGGSGGGGSQVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 555 - protein, 478 aa, synthetic construct
QVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSSGGGGSGGGGSGGGGSGGGGSDIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 556 - protein, 478 aa, synthetic construct
EVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSSGGGGSGGGGSGGGGSGGGGSDIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 557 - protein, 478 aa, synthetic construct; identical to SEQ ID NO: 556

SEQ ID NO: 558 - protein, 486 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTNFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 559 - protein, 486 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 560 - protein, 486 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTQFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 561 - protein, 486 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTNFALTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 562 - protein, 486 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 563 - protein, 486 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSSGYNYLDWYLQKPGQSPQLLISLGSIRASGVPDRFSGSGSGTDFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 564 - protein, 486 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSQGYNYLDWYLQKPGQSPQLLISLGSIRASGVPDRFSGSGSGTDFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 565 - protein, 486 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNAYNYLDWYLQKPGQSPQLLISLGSIRASGVPDRFSGSGSGTDFTLTISRVEAEDVGFYYCMQALQTPRTFGCGTKVDITGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 566 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 567 - protein, 121 aa, synthetic construct
QVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSS

SEQ ID NO: 568 - protein, 107 aa, synthetic construct
DIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIK

SEQ ID NO: 569 - protein, 248 aa, synthetic construct
DIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIKGGGGSGGGGSGGGGSGGGGSQVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSS

SEQ ID NO: 570 - protein, 248 aa, synthetic construct
EVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSSGGGGSGGGGSGGGGSGGGGSDIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIK

SEQ ID NO: 571 - protein, 249 aa, synthetic construct
QVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSGGGGSGGGGSGGGGSGGGGSDIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIK

SEQ ID NO: 572 - protein, 249 aa, synthetic construct
EVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSGGGGSGGGGSGGGGSGGGGSDIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIK

SEQ ID NO: 573 - protein, 249 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGGGGSGGGGSGGGGSGGGGSQVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSS

SEQ ID NO: 574 - protein, 250 aa, synthetic construct
QVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSSGGGGSGGGGSGGGGSGGGGSDIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIK

SEQ ID NO: 575 - protein, 250 aa, synthetic construct
EVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSSGGGGSGGGGSGGGGSGGGGSDIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIK

SEQ ID NO: 576 - protein, 250 aa, synthetic construct
DIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIKGGGGSGGGGSGGGGSGGGGSQVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSS

SEQ ID NO: 577 - protein, 258 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTNFTLTISRVEAEDVGFYYCMQALQTPRTFGCGTKVDIT

SEQ ID NO: 578 - protein, 258 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDIT

SEQ ID NO: 579 - protein, 258 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTQFTLTISRVEAEDVGFYYCMQALQTPRTFGCGTKVDIT

SEQ ID NO: 580 - protein, 258 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTNFALTISRVEAEDVGFYYCMQALQTPRTFGCGTKVDIT

SEQ ID NO: 581 - protein, 258 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDIT

SEQ ID NO: 582 - protein, 258 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSSGYNYLDWYLQKPGQSPQLLISLGSIRASGVPDRFSGSGSGTDFTLTISRVEAEDVGFYYCMQALQTPRTFGCGTKVDIT

SEQ ID NO: 583 - protein, 258 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSQGYNYLDWYLQKPGQSPQLLISLGSIRASGVPDRFSGSGSGTDFTLTISRVEAEDVGFYYCMQALQTPRTFGCGTKVDIT

SEQ ID NO: 584 - protein, 258 aa, synthetic construct
EVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSSGGGGSGGGGSGGGGSGGGGSEIVMTQSPLSLPVTPGEPASISCRSSQSLLHSNAYNYLDWYLQKPGQSPQLLISLGSIRASGVPDRFSGSGSGTDFTLTISRVEAEDVGFYYCMQALQTPRTFGCGTKVDIT

SEQ ID NO: 585 - protein, 258 aa, synthetic construct
EIVMTQSPLSLPVTPGEPASISCRSSQSLLHSNAYNYLDWYLQKPGQSPQLLISLGSIRASGVPDRFSGSGSGTDFTLTISRVEAEDVGFYYCMQALQTPRTFGCGTKVDITGGGGSGGGGSGGGGSGGGGSEVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 586 - protein, 248 aa, synthetic construct
QVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSSGGGGSGGGGSGGGGSGGGGSDIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIK

SEQ ID NO: 587 - protein, 119 aa, synthetic construct
EVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTV

SEQ ID NO: 588 - protein, 107 aa, synthetic construct; identical to SEQ ID NO: 568

SEQ ID NO: 589 - protein, 248 aa, synthetic construct
DIRMTQSPSTLSASVGDRVTITCWASQSISSWLAWYQQKPGKAPKLLIYKASSLESGVPSRFSGSGSGTEFTLTISSLQPDDFATYYCQQYNSYSYTFGCGTKLEIKGGGGSGGGGSGGGGSGGGGSEVQLVQSGGGLVQPGGSLRLSCAASGFTFSSYAMSWVRQAPGKCLEWVSAIFNSGGSTYYADSVKGRFTVSRDNSKNTLYLQMNSLRAEDTALYYCAKDLGGYNYGLFDYWGQGTLVTVSS

SEQ ID NO: 590 - protein, 121 aa, synthetic construct
QVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSS

SEQ ID NO: 591 - protein, 108 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIK

SEQ ID NO: 592 - protein, 249 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGGGSGGGGSGGGGSGGGGSQVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSG

SEQ ID NO: 593 - protein, 121 aa, synthetic construct
EVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSS

SEQ ID NO: 594 - protein, 108 aa, synthetic construct; identical to SEQ ID NO: 591

SEQ ID NO: 595 - protein, 249 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGGGGSGGGGSGGGGSGGGGSEVQLVQSGGGLVQPGGSQRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSS

SEQ ID NO: 596 - protein, 121 aa, synthetic construct; identical to SEQ ID NO: 590

SEQ ID NO: 597 - protein, 108 aa, synthetic construct; identical to SEQ ID NO: 591

SEQ ID NO: 598 - protein, 249 aa, synthetic construct; identical to SEQ ID NO: 571

SEQ ID NO: 599 - protein, 124 aa, synthetic construct
QVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSS

SEQ ID NO: 600 - protein, 106 aa, synthetic construct
DIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIK

SEQ ID NO: 601 - protein, 250 aa, synthetic construct; identical to SEQ ID NO: 576

SEQ ID NO: 602 - protein, 124 aa, synthetic construct
EVQLQESGPGLVRPSGTLSLTCAVSGGSISSPTWWSWVRQPPGKCLEWIGEIHPSGRTNYNPSLKSRVTISVDKSKNQFSLKLGSVTAADTAVYYCAREGFYYGSGNYYYFDYWGQGTLVTVSS

SEQ ID NO: 603 - protein, 106 aa, synthetic construct; identical to SEQ ID NO: 600

SEQ ID NO: 604 - protein, 232 aa, synthetic construct
DIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIKGGGGSGGGGSGGGGSGGGGSDIVMTQTPATLSASVGDRVTITCRASQSVRSNLAWYQQKPGQAPRLLIYGASTRATGIPARFSGSGSGTEFTLTISSLQSEDFAVYYCQQYNNWPTFGCGTRLEIK

SEQ ID NO: 605 - protein, 124 aa, synthetic construct; identical to SEQ ID NO: 599

SEQ ID NO: 606 - protein, 106 aa, synthetic construct; identical to SEQ ID NO: 600

SEQ ID NO: 607 - protein, 250 aa, synthetic construct; identical to SEQ ID NO: 574

SEQ ID NO: 608 - protein, 126 aa, synthetic construct
QVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 609 - protein, 112 aa, synthetic construct
EIVMTQSPLSLPVTPGEPASISCRSSQSLLHSNGYNYLDWYLQKPGQSPQLLISLGSIRASGVPDRFSGSGSGTNFTLTISRVEAEDVGFYYCMQALQTPRTFGCGTKVDIT

SEQ ID NO: 610 - protein, 244 aa, synthetic construct
EIVMTQSPLSLPVTPGEPASISCRSSQSLLHSNGYNYLDWYLQKPGQSPQLLISLGSIRASGVPDRFSGSGSGTNFTLTISRVEAEDVGFYYCMQALQTPRTFGCGTKVDITGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTNFTLTISRVEAEDVGFYYCMQALQTPRTFGCGTKVDIT

SEQ ID NO: 611 - protein, 126 aa, synthetic construct; identical to SEQ ID NO: 608

SEQ ID NO: 612 - protein, 112 aa, synthetic construct
EIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDIT

SEQ ID NO: 613 - protein, 258 aa, synthetic construct
EIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDITGGGGSGGGGSGGGGSGGGGSQVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 614 - protein, 126 aa, synthetic construct; identical to SEQ ID NO: 608

SEQ ID NO: 615 - protein, 112 aa, synthetic construct
EIVMTQSPLSLPVTPGEPASISCRSSQSLLHSNGYNYLDWYLQKPGQSPQLLISLGSIRASGVPDRFSGSGSGTQFTLTISRVEAEDVGFYYCMQALQTPRTFGCGTKVDIT

SEQ ID NO: 616 - protein, 258 aa, synthetic construct
EIVMTQSPLSLPVTPGEPASISCRSSQSLLHSNGYNYLDWYLQKPGQSPQLLISLGSIRASGVPDRFSGSGSGTQFTLTISRVEAEDVGFYYCMQALQTPRTFGCGTKVDITGGGGSGGGGSGGGGSGGGGSQVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 617 - protein, 126 aa, synthetic construct; identical to SEQ ID NO: 608

SEQ ID NO: 618 - protein, 112 aa, synthetic construct
EIVMTQSPLSLPVTPGEPASISCRSSQSLLHSNGYNYLDWYLQKPGQSPQLLISLGSIRASGVPDRFSGSGSGTNFALTISRVEAEDVGFYYCMQALQTPRTFGCGTKVDIT

SEQ ID NO: 619 - protein, 244 aa, synthetic construct
EIVMTQSPLSLPVTPGEPASISCRSSQSLLHSNGYNYLDWYLQKPGQSPQLLISLGSIRASGVPDRFSGSGSGTNFALTISRVEAEDVGFYYCMQALQTPRTFGCGTKVDITGGGGSGGGGSGGGGSGGGGSEIVMTQSPLSLPVTPGEPASISCRSSQSLLHSNGYNYLDWYLQKPGQSPQLLISLGSIRASGVPDRFSGSGSGTNFALTISRVEAEDVGFYYCMQALQTPRTFGCGTKVDIT

SEQ ID NO: 620 - protein, 126 aa, synthetic construct; identical to SEQ ID NO: 471

SEQ ID NO: 621 - protein, 112 aa, synthetic construct; identical to SEQ ID NO: 612

SEQ ID NO: 622 - protein, 258 aa, synthetic construct
EIVMTQSPLSLPVTPGEPASISCRSSQSLLHSNGYNYLDWYLQKPGQSPQLLISLGSIRASGVPDRFSGSGSGTDFTLTISRVEAEDVGFYYCMQALQTPRTFGCGTKVDITGGGGSGGGGSGGGGSGGGGSEVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 623 - protein, 126 aa, synthetic construct; identical to SEQ ID NO: 471

SEQ ID NO: 624 - protein, 112 aa, synthetic construct
EIVMTQSPLSLPVTPGEPASISCRSSQSLLHSSGYNYLDWYLQKPGQSPQLLISLGSIRASGVPDRFSGSGSGTDFTLTISRVEAEDVGFYYCMQALQTPRTFGCGTKVDIT

SEQ ID NO: 625 - protein, 16 aa, synthetic construct
RSSQSLLHSNAYNYLD

SEQ ID NO: 626 - protein, 258 aa, synthetic construct
EIVMTQSPLSLPVTPGEPASISCRSSQSLLHSSGYNYLDWYLQKPGQSPQLLISLGSIRASGVPDRFSGSGSGTDFTLTISRVEAEDVGFYYCMQALQTPRTFGCGTKVDITGGGGSGGGGSGGGGSGGGGSEVQLVESGGDVVQPGRSLRLSCAASGFILSNYGMHWVRQAPGKCLEWVAAMWYDGSNNYYEDSVKGRFTISRDNSKNTLYLQMNSLRAEDTAVYYCARERVSRHFDWHYYYGMDVWGQGTTVTVSS

SEQ ID NO: 627 - protein, 126 aa, synthetic construct; identical to SEQ ID NO: 471

SEQ ID NO: 628 - protein, 112 aa, synthetic construct
EIVMTQSPLSLPVTPGEPASISCRSSQSLLHSQGYNYLDWYLQKPGQSPQLLISLGSIRASGVPDRFSGSGSGTDFTLTISRVEAEDVGFYYCMQALQTPRTFGCGTKVDIT

SEQ ID NO: 629 - protein, 16 aa, synthetic construct
RSSQSLLHSQGYNYLD

SEQ ID NO: 630 - protein, 16 aa, synthetic construct
RSSQSLLHSSGYNYLD

SEQ ID NO: 631 - protein, 112 aa, synthetic construct
EIVMTQSPLSLPVTPGEPASISCRSSQSLLHSSGYNYLDWYLQKPGQSPQLLISLGSIRASGVPDRFSGSGSGTDFTLTISRVEAEDVGFYYCMQALQTPRTFGQGTKVDIT

SEQ ID NO: 632 - protein, 16 aa, synthetic construct
RSSQSLLHSSGYNYLD

SEQ ID NO: 633 - protein, 112 aa, synthetic construct
EIVMTQSPLSLPVTPGEPASISCRSSQSLLHSQGYNYLDWYLQKPGQSPQLLISLGSIRASGVPDRFSGSGSGTDFTLTISRVEAEDVGFYYCMQALQTPRTFGQGTKVDIT

SEQ ID NO: 634 - protein, 16 aa, synthetic construct
RSSQSLLHSQGYNYLD

SEQ ID NO: 635 - protein, 112 aa, synthetic construct
EIVMTQSPLSLPVTPGEPASISCRSSQSLLHSNAYNYLDWYLQKPGQSPQLLISLGSIRASGVPDRFSGSGSGTDFTLTISRVEAEDVGFYYCMQALQTPRTFGQGTKVDIT

SEQ ID NO: 636 - protein, 16 aa, synthetic construct
RSSQSLLHSNAYNYLD

SEQ ID NO: 637 - protein, 107 aa, synthetic construct
DIQMTQSPSTLSASVGDRVTITCWASQSISSWLAWYQQKPGKAPKLLIYKASSLESGVPSRFSGSGSGTEFTLTISSLQPDDFATYYCQQYNSYSYTFGQGTKLEIK

SEQ ID NO: 638 - protein, 121 aa, synthetic construct
QVQLVQSGGGLVQPGGSLRLSCAASGFTFTSYAMSWVRQAPGKGLEWVSAISGTGDSTFYADSVKGRFTFSRDNSKNTLYLQMNSLRAEDTAVYYCAKDLGWLQYGLFDYWGQGTLVTVSS

SEQ ID NO: 639 - protein, 121 aa, synthetic construct
EVQLQESGAELMKPGASVKISCKATGYTFSTYWIEWVKPRPGHGLEWIGEILPGTGTTNYNEKFKGKATFTADTSSNTAYMQLSSLTSEDSAVYYCATLNGHGDYWYFDVWGAGTTVTVSS

SEQ ID NO: 640 - protein, 7 aa, synthetic construct
GYTFSTY

SEQ ID NO: 641 - protein, 5 aa, synthetic construct
TYWIE

SEQ ID NO: 642 - protein, 6 aa, synthetic construct
LPGTGT

SEQ ID NO: 643 - protein, 17 aa, synthetic construct
EILPGTGTTNYNEKFKG

SEQ ID NO: 644 - protein, 12 aa, synthetic construct
LNGHGDYWYFDV

SEQ ID NO: 645 - protein, 121 aa, synthetic construct
QVQLQQSGAELMKPGASVKISCKATGYTFSTYWIEWVKPRPGHGLEWIGEILPGTGTTNYNEKFKGKATFTADTSSNTAYMQLSSLTSEDSAVYYCATLNGHGDYWYFDVWGAGTTVTVSS

SEQ ID NO: 646 - protein, 109 aa, synthetic construct
QAVVTQESALTTSPGETVTLTCRSSTGGVTTSNYANWVQEKPDHLFTGLIGGTNNRAPGVPARFSGSLIGDKAALTITGAQTEDEAIYFCALWYSNHLVFGGGTKLTVL

SEQ ID NO: 647 - protein, 14 aa, synthetic construct
RSSTGGVTTSNYAN

SEQ ID NO: 648 - protein, 7 aa, synthetic construct
GTNNRAP

SEQ ID NO: 649 - protein, 9 aa, synthetic construct
ALWYSNHLV

SEQ ID NO: 650 - protein, 121 aa, synthetic construct
EVQLQESGPELVKPGASLKISCKASGYSFTDYTMNWVKQSHGKNLEWIGLINPYNGGTTYNQKFKDMATLTVDKSSSTAYMELLSLTSEDSAVYYCARSEYGHSYWYFDVWGAGTTVTVSP

SEQ ID NO: 651 - protein, 7 aa, synthetic construct
GYSFTDY

SEQ ID NO: 652 - protein, 5 aa, synthetic construct
DYTMN

SEQ ID NO: 653 - protein, 6 aa, synthetic construct
NPYNGG

SEQ ID NO: 654 - protein, 17 aa, synthetic construct
LINPYNGGTTYNQKFKD

SEQ ID NO: 655 - protein, 12 aa, synthetic construct
SEYGHSYWYFDV

SEQ ID NO: 656 - protein, 121 aa, synthetic construct
EVQLQQSGPELVKPGASLKISCKASGYSFTDYTMNWVKQSHGKNLEWIGLINPYNGGTTYNQKFKDMATLTVDKSSSTAYMELLSLTSEDSAVYYCARSEYGHSYWYFDVWGAGTTVTVSS

SEQ ID NO: 657 - protein, 106 aa, synthetic construct
QIVLTQSPAIMSASPGEKVTLTCSPSSSVTYMHWYQQKSGTSPKRWIYDTSKLASGVPARFSGIGSGTSYSLTINSMEAEDAATYYCQQWNNYPYTFGGGTRLEIK

SEQ ID NO: 658 - protein, 10 aa, synthetic construct
SPSSSVTYMH

SEQ ID NO: 659 - protein, 7 aa, synthetic construct
DTSKLAS

SEQ ID NO: 660 - protein, 9 aa, synthetic construct
QQWNNYPYT

SEQ ID NO: 661 - protein, 106 aa, synthetic construct
DIQMTQSPASLSASVGDTVTITCRASENIYSYFAWYQQKQGKSPRLLVYNARALAEGVPSRFSGSGSGTQFSKINSLQPEDFGSYYCQHLYGAPFTFGSGTKLEIK

SEQ ID NO: 662 - protein, 11 aa, synthetic construct
RASENIYSYFA

SEQ ID NO: 663 - protein, 7 aa, synthetic construct
NARALAE

SEQ ID NO: 664 - protein, 9 aa, synthetic construct
QHLYGAPFT

SEQ ID NO: 665 - protein, 119 aa, synthetic construct
QIQLVQSGPELKKPGETVKISCKASGYSFTKYGMNWVKQAPGKGLKWMGWINTYSGEPTYADDFEGRFAFSLETSANTAYLQINNLKNEDMATYFCARGGGFDYGFDYWGQGTTLTVST

SEQ ID NO: 666 - protein, 7 aa, synthetic construct
GYSFTKY

SEQ ID NO: 667 - protein, 5 aa, synthetic construct
KYGMN

SEQ ID NO: 668 - protein, 6 aa, synthetic construct
NTYSGE

SEQ ID NO: 669 - protein, 17 aa, synthetic construct
WINTYSGEPTYADDFEG

SEQ ID NO: 670 - protein, 10 aa, synthetic construct
GGGFDYGFDY

SEQ ID NO: 671 - protein, 106 aa, synthetic construct
QIVLTQSPAIMSASPGEKVTMTCSASSSVSYIHWYRQRSGTSPKRWIYDTSKLASGVPARFSGSGSGTSYSLTISTMEAEDAATYYCQQWSNYPYTFGGGTKLEIK

SEQ ID NO: 672 - protein, 10 aa, synthetic construct
SASSSVSYIH

SEQ ID NO: 673 - protein, 9 aa, synthetic construct
QQWSNYPYT

SEQ ID NO: 674 - protein, 121 aa, synthetic construct
EVQLQQSGPELVKPGASMKISCKASGYSFTDYTMNWVKQSHGKNLEWIGHIYPYNGGTTYNQKFQDKASLTADKSSSTAYMELLSLTSEDSAVYYCARGEFLRSYWYFDVWGAGTLVAVSS

SEQ ID NO: 675 - protein, 6 aa, synthetic construct
YPYNGG

SEQ ID NO: 676 - protein, 17 aa, synthetic construct
HIYPYNGGTTYNQKFQD

SEQ ID NO: 677 - protein, 12 aa, synthetic construct
GEFLRSYWYFDV

SEQ ID NO: 678 - protein, 106 aa, synthetic construct
QIVLTQSPAIMSASPGEKVTLTCSPSSSVTYMHWYQQKSGTSPKRWIYDTSKLASGVPARFSGIGSGTSYSLTINSMEAEDAATYYCQQWNNYPYTFGGGTKLEIK

SEQ ID NO: 679 - protein, 107 aa, synthetic construct
DIQMTQSPASLSASVGETVTITCRGSENIYSYLTWYQQKQGKSPQLLVYNAKTLAEGVPSRFSGSGSGTQFSLKINSLQPEDFGRYYCQHLYSSPYTFGGGTKLEIK

SEQ ID NO: 680 - protein, 11 aa, synthetic construct
RGSENIYSYLT

SEQ ID NO: 681 - protein, 7 aa, synthetic construct
NAKTLAE

SEQ ID NO: 682 - protein, 9 aa, synthetic construct
QHLYSSPYT

SEQ ID NO: 683 - protein, 107 aa, synthetic construct
DIQMTQSPASLSASVGDTVTITCRASENIYSYFAWYQQKQGKSPRLLVYNARALAEGVPSRFSGSGSGTQFSLKINSLQPEDFGSYYCQHLYGAPFTFGSGTKLEIK

SEQ ID NO: 684 - protein, 120 aa, synthetic construct
QVQLQESGPGLVTPSGTLSLTCAVSGDSISSSHWWSWVRQPPGKGLEWIGEIYHSGITNYRSSLKSRVTLSVDKSKNQFSLKLTSVTAADTAVYYCARGGSGNYEAFDIWGQGTLFTVSS

SEQ ID NO: 685 - protein, 8 aa, synthetic construct
GDSISSSH

SEQ ID NO: 686 - protein, 6 aa, synthetic construct
SSHWWS

SEQ ID NO: 687 - protein, 5 aa, synthetic construct
YHSGI

SEQ ID NO: 688 - protein, 16 aa, synthetic construct
EIYHSGITNYRSSLKS

SEQ ID NO: 689 - protein, 11 aa, synthetic construct
GGSGNYEAFDI

SEQ ID NO: 690 - protein, 107 aa, synthetic construct
EIVMTQSPATLSVSPGERATLSCRASQSVRSNLAWYLQKPGQAPRLLIYGASTRATGIPARFSGSGSGTEFTLTISSLQSEDFAVYYCQQYNNWPLTFGGGTKVEIK

SEQ ID NO: 691 - protein, 115 aa, synthetic construct
QVQLQESGPGLVKPSETLSLTCTVSGGPIYSYYWSWIRQPPGKGLEWIGFIYSSGSTNYNPSLKSRVTISIDTSKSQFSLRLSSVTAADTAVYYCARGGDAFDIWGQGTMVTVSS

SEQ ID NO: 692 - protein, 119 aa, synthetic construct
QIQLVQSGPELKKPGETVKISCKASGYTFTNYGMNWVKQAPGKGLKWMGWINTYTGEPTYTDDFKGRFAFSLETSASTAYLQITNLKNEDTATYFCARGDGFDRGFAYWGQGTLVTVSA

SEQ ID NO: 693 - protein, 7 aa, synthetic construct
GYTFTNY

SEQ ID NO: 694 - protein, 5 aa, synthetic construct
NYGMN

SEQ ID NO: 695 - protein, 6 aa, synthetic construct
NTYTGE

SEQ ID NO: 696 - protein, 17 aa, synthetic construct
WINTYTGEPTYTDDFKG

SEQ ID NO: 697 - protein, 10 aa, synthetic construct
GDGFDRGFAY

SEQ ID NO: 698 - protein, 106 aa, synthetic construct
QIVLTQSPAIMSASPGEKVTLTCSPSSSVTYMHWYQQKSGTSPKRWIYDTSKLASGVPARFSGIGSGTSYSLTINSMEAEDAATYYCQQWNNYPYTFGGGTRLEIT

SEQ ID NO: 699 - protein, 119 aa, synthetic construct
QIQLVQSGPELKKPGETVKISCKASGYSFTKYGMNWVKQAPGKGLKWMGWINTYSGEPTYADDFEGRFAFSLETSANTAYLQINNLKNEDMATYFCARGGGFDYGFDYWGQGTTLTVSS

SEQ ID NO: 700 - protein, 249 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPIGAAAGWFDPWGQGTLVTVSS

SEQ ID NO: 701 - protein, 6 aa, synthetic construct
SSSSSY

SEQ ID NO: 702 - protein, 479 aa, synthetic construct
DIVMTQSPDSLAVSLGERATINCKSSHSLLYGNFQNNYLAWYQQKPGQPPKLLIYWASTRESGVPDRFSGSGSGTDFTLTISSLQAEDVAVYYCQQYYSYPYTFGCGTKLEIKGGGGSGGGGSGGGGSGGGGSQVQLVQSGAEVKKPGASVKVSCKASGYTFTAYTIHWVRQAPGQCLEWMGYINPSSGYTEYNQKFKDRTTLTADTSIPTAYMELSRLRSDDTAVYYCTREGGLLWFDYWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 703 - protein, 251 aa, synthetic construct
DIVMTQSPDSLAVSLGERATINCKSSHSLLYGNFQNNYLAWYQQKPGQPPKLLIYWASTRESGVPDRFSGSGSGTDFTLTISSLQAEDVAVYYCQQYYSYPYTFGCGTKLEIKGGGGSGGGGSGGGGSGGGGSQVQLVQSGAEVKKPGASVKVSCKASGYTFTAYTIHWVRQAPGQCLEWMGYINPSSGYTEYNQKFKDRTTLTADTSIPTAYMELSRLRSDDTAVYYCTREGGLLWFDYWGQGTLVTVSS

SEQ ID NO: 704 - protein, 118 aa, synthetic construct
QVQLVQSGAEVKKPGASVKVSCKASGYTFTAYTIHWVRQAPGQCLEWMGYINPSSGYTEYNQKFKDRTTLTADTSIPTAYMELSRLRSDDTAVYYCTREGGLLWFDYWGQGTLVTVSS

SEQ ID NO: 705 - protein, 113 aa, synthetic construct
DIVMTQSPDSLAVSLGERATINCKSSHSLLYGNFQNNYLAWYQQKPGQPPKLLIYWASTRESGVPDRFSGSGSGTDFTLTISSLQAEDVAVYYCQQYYSYPYTFGCGTKLEIK

SEQ ID NO: 706 - protein, 477 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGGGGSGGGGSGGGGSGGGGSQVQLVQSGGGLVQPGGSLRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 707 - protein, 249 aa, synthetic construct
DIQLTQSPATLSVSPGERATLSCRASQSVSSSYLAWYQQKPGQAPRLLIYGASSRATGIPDRFSGSGSGTDFTLTISRLEPEDFAVYYCQQYNNWPLTFGCGTKVEIKGGGGSGGGGSGGGGSGGGGSQVQLVQSGGGLVQPGGSLRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSS

SEQ ID NO: 708 - protein, 121 aa, synthetic construct
QVQLVQSGGGLVQPGGSLRLSCAASGFTFTSYAMSWVRQAPGKCLEWVSAISGTGDSTFYADSVKGRFTFSRDNSKNTLYLQMNSLRAEDTAVYYCAKDLGWLQYGLFDYWGQGTLVTVSS

SEQ ID NO: 709 - protein, 476 aa, synthetic construct
DIQMTQSPSTLSASVGDRVTITCWASQSISSWLAWYQQKPGKAPKLLIYKASSLESGVPSRFSGSGSGTEFTLTISSLQPDDFATYYCQQYNAYSYTFGCGTKLEIKGGGGSGGGGSGGGGSGGGGSQVQLVQSGGGLVQPGGSLRLSCAASGFTFSSYAMSWVRQAPGKCLEWVSAIFSSGGSTYYADSVKGRFTVSRDNSKNTLYLQMNSLRAEDTALYYCAKDLGGYNYGLFDYWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 710 - protein, 248 aa, synthetic construct
DIQMTQSPSTLSASVGDRVTITCWASQSISSWLAWYQQKPGKAPKLLIYKASSLESGVPSRFSGSGSGTEFTLTISSLQPDDFATYYCQQYNAYSYTFGCGTKLEIKGGGGSGGGGSGGGGSGGGGSQVQLVQSGGGLVQPGGSLRLSCAASGFTFSSYAMSWVRQAPGKCLEWVSAIFSSGGSTYYADSVKGRFTVSRDNSKNTLYLQMNSLRAEDTALYYCAKDLGGYNYGLFDYWGQGTLVTVSS

SEQ ID NO: 711 - protein, 121 aa, synthetic construct; identical to SEQ ID NO: 708

SEQ ID NO: 712 - protein, 108 aa, synthetic construct; identical to SEQ ID NO: 591

SEQ ID NO: 713 - protein, 479 aa, synthetic construct
DIVMTQSPDSLAVSLGERATINCKSSHSLLYGNFQNNYLAWYQQKPGQPPKLLIYWASTRESGVPDRFSGSGSGTDFTLTISSLQAEDVAVYYCQQYYSYPYTFGCGTKLEIKGGGGSGGGGSGGGGSGGGGSQVQLVQSGAEVKKPGASVKVSCKASGYTFTAYTIHWVRQAPGQCLEWMGYINPSSGYTEYNQKFKDRTTLTADTSISTAYMELSRLRSDDTAVYYCTREGGLLWFDYWGQGTLVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 714 - protein, 251 aa, synthetic construct
DIVMTQSPDSLAVSLGERATINCKSSHSLLYGNFQNNYLAWYQQKPGQPPKLLIYWASTRESGVPDRFSGSGSGTDFTLTISSLQAEDVAVYYCQQYYSYPYTFGCGTKLEIKGGGGSGGGGSGGGGSGGGGSQVQLVQSGAEVKKPGASVKVSCKASGYTFTAYTIHWVRQAPGQCLEWMGYINPSSGYTEYNQKFKDRTTLTADTSISTAYMELSRLRSDDTAVYYCTREGGLLWFDYWGQGTLVTVSS

SEQ ID NO: 715 - protein, 118 aa, synthetic construct
QVQLVQSGAEVKKPGASVKVSCKASGYTFTAYTIHWVRQAPGQCLEWMGYINPSSGYTEYNQKFKDRTTLTADTSISTAYMELSRLRSDDTAVYYCTREGGLLWFDYWGQGTLVTVSS

SEQ ID NO: 716 - protein, 113 aa, synthetic construct; identical to SEQ ID NO: 705

SEQ ID NO: 717 - protein, 6 aa, synthetic construct
FSSGGS

SEQ ID NO: 718 - protein, 17 aa, synthetic construct
AIFSSGGSTYYADSVKG

SEQ ID NO: 719 - protein, 121 aa, synthetic construct
QVQLVQSGGGLVQPGGSLRLSCAASGFTFSSYAMSWVRQAPGKGLEWVSAIFNSGGSTYYADSVKGRFTVSRDNSKNTLYLQMNSLRAEDTALYYCAKDLGGYNYGLFDYWGQGTLVTVSS

SEQ ID NO: 720 - protein, 17 aa, synthetic construct
EISGSGDRTSYADAVKG

(19) United States
(12) Patent Application Publication
(10) Pub. No.: US 2024/0124607 A1
(43) Pub. Date: Apr. 18, 2024

(54) PROTEINS BINDING NKG2D, CD16, AND CEACAM5

(71) Applicants: Merck Sharp & Dohme LLC, Rahway, NJ (US); Dragonfly Therapeutics, Inc., Waltham, MA (US)

(72) Inventors: Amy M. Beebe, Half Moon Bay, CA (US); Laurence Fayadat-Dilman, San Carlos, CA (US); Veronica M. Juan, Redwood City, CA (US); Gregory P. Chang, Lynnfield, MA (US); Souvik Chattopadhyay, Sharon, MA (US); Ann F. Cheung, Lincoln, MA (US); Asya Grinberg, Lexington, MA (US); Pyae P. Hein, Melsore, MA (US); Nicolai Wagtmann, Concord, MA (US)

(21) Appl. No.: 18/366,876

(22) Filed: Aug. 8, 2023

Related U.S. Application Data
(60) Provisional application No. 63/396,910, filed on Aug. 10, 2022.

(51) Int. Cl. C07K 16/30; A61P 35/00; C07K 16/28

(52) U.S. Cl. CPC C07K 16/3007; A61P 35/00; C07K 16/2818; C07K 16/2827; C07K 16/283; C07K 16/2851; C07K 2317/31; C07K 2317/53; C07K 2317/622

(57) ABSTRACT

Multi-specific binding proteins that bind NKG2D, CD16, and CEACAM5 are described, as well as pharmaceutical compositions and therapeutic methods useful for the treatment of cancer

### FIELD OF THE INVENTION

The invention relates to multi-specific binding proteins that bind to NKG2D, CD16, and CEACAM5.

### REFERENCE TO SEQUENCE LISTING SUBMITTED ELECTRONICALLY

The sequence listing of the present application is submitted electronically via EFS-Web as an ASCII formatted sequence listing with a file name “25565_SL.xml”, creation date of Feb. 1, 2023, and a size of 715 KB. This sequence listing submitted via EFS-Web is part of the specification and is herein incorporated by reference in its entirety.

### BACKGROUND

Despite substantial research efforts, cancer continues to be a significant clinical and financial burden in countries across the globe. According to the World Health Organization F(WHO), it is the second leading cause of death. Surgery, radiation therapy, chemotherapy, biological therapy, immunotherapy, hormone therapy, stem-cell transplantation, and precision medicine are among the existing treatment modalities. Despite extensive research in these areas, a highly effective, curative solution, particularly for the most aggressive cancers, has yet to be identified. Furthermore, many of the existing anti-cancer treatment modalities have substantial adverse side effects.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perform and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16, an Fc receptor (Fcγ receptor III) present on the surface of the NK cell. The overall sensitivity of NK cells to activation depends on the sum of stimulatory and inhibitory signals. NKG2D is a type-II transmembrane protein that is expressed by essentially all natural killer cells where NKG2D serves as an activating receptor. NKG2D is also be found on T cells where it acts as a costimulatory receptor. The ability to modulate NK cell function via NKG2D is useful in various therapeutic contexts including malignancy.

Carcinoembryonic Antigen-Related Cell Adhesion Molecule 5 (CEACAM5), also referred to as Meconium Antigen 100, CEA, Carcinoembryonic Antigen, CD66e or CD66e Antigen, is a member of the immunoglobulin superfamily. It is a large cell surface glycoprotein, and mainly serves as a cell adhesion molecule mediating intercellular contact. Besides its functions in cell adhesion and migration, CEACAM5 is found to be over-expressed in a high percentage of human cancers, including 90% of gastrointestinal, colorectal and pancreatic cancers, 70% of non-small cell lung cancer cells, and 50% of breast cancers. Overexpression of CEACAM5 has been shown to positively correlate with tumorigenicity and enhanced tumor invasiveness.

Although proteins (e.g., antibodies) that bind CEACAM5 are under development as potential anticancer therapies, they face significant challenges. Some of these challenges, such as high levels of homology with other CEACAM family members, low percent homology with cynomolgus (cyno) CEACAM5, and the highly glycosylated nature of the protein, lead to difficulty achieving both monospecificity for human CEACAM5 and cross-reactivity with cyno CEACAM5. These challenges highlight a need in the field for new and useful antibodies for use in treatment of CEACAM5-related cancer.

### SUMMARY

The invention provides multi-specific binding proteins that bind to the NKG2D receptor and CD16 on natural killer cells, and tumor-associated antigen CEACAM5 (Carcinoembryonic Antigen-Related Cell Adhesion Molecule 5). Such proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. Formulations containing any one of the proteins described herein; cells containing one or more nucleic acids expressing the proteins, and methods of enhancing tumor cell death using the proteins are also provided.

Accordingly, in one aspect, the present disclosure provides a protein comprising a first antigen-binding site that binds NKG2D, a second antigen-binding site that binds CEACAM5, and a third antigen-binding site, or an antibody Fc domain or a portion thereof, in each case (i.e., third antigen-binding site, antibody Fc domain, or portion) that binds CD16.

In some embodiments, the second antigen-binding site that binds CEACAM5 comprises a heavy-chain variable domain (VH) comprising a complementarity-determining region (CDR) 1 (CDRH1), CDRH2, and CDRH3, wherein CDRH1 comprises an amino acid sequence selected from the group consisting of: SEQ ID NOs: 3 and 102, CDRH2 comprises an amino acid sequence selected from the group consisting of: SEQ ID NOs: 37, 104, and 718, and CDRH3 comprises an amino acid sequence selected from the group consisting of: SEQ ID NOs: 6, 38, and 105.

In some embodiments, the second antigen-binding site that binds CEACAM5 comprises a light-chain variable domain (VL) comprising a CDR 1 (CDL1), CDRL2, and CDRL3, wherein the CDRL1 comprises an amino acid sequence selected from the group consisting of: SEQ ID NOs: 7, 40, and 107, CDRL2 comprises an amino acid sequence selected from the group consisting of: SEQ ID NOs: 8, 41, and 108, and CDRL3 comprises an amino acid sequence selected from the group consisting of: SEQ ID NOs: 9, 42, and 109.

In some embodiments, the CDRH1, CDRH2, and CDRH3 of the second antigen-binding site are: (i) SEQ ID NOs: 3, 37, and 38, respectively; (ii) SEQ ID NOs: 3, 718, and 6, respectively; or (iii) SEQ ID NOs: 102, 104, and 105, respectively.

In some embodiments, the CDRL1, CDRL2, and CDRL3 of the second antigen-binding site are: (i) SEQ ID NOs: 7, 8, and 9, respectively; (ii) SEQ ID NOs: 40, 41, and 42, respectively; or (iii) SEQ ID NOs: 107, 108, and 109, respectively.

In some embodiments, the CDRH1, CDRH2, CDRH3, CDRL1, CDRL2, and CDRL3 are: (i) SEQ ID NOs: 3, 37, 38, 40, 41, and 42, respectively; (ii) SEQ ID NOs: 3, 718, 6, 7, 8, and 9, respectively; or (iii) SEQ ID NOs: 102, 104, 105, 107, 108, and 109, respectively.

In some embodiments, the VH comprises an amino acid sequence selected from the group consisting of: SEQ ID NOs: 704, 708, 711, and 715; and the VL comprises an amino acid sequence selected from the group consisting of: SEQ ID NOs: 591, 705, 712, and 716.

In some embodiments, the VH and VL are: (i) SEQ ID NOs: 704 and 705, respectively; (ii) SEQ ID NOs: 708 and 591, respectively; (iii) SEQ ID NOs: 711 and 712, respectively; or (iv) SEQ ID NOs: 715 and 716, respectively.

In some embodiments, the second antigen-binding site is a single-chain variable fragment (scFv), wherein the scFv comprises an amino acid sequence selected from the group consisting of: SEQ ID NOs:703, 707, 710, and 714.

In some embodiments, the second antigen-binding site binds a human CEACAM5 variant comprising the amino acid sequence of SEQ ID NO:391.

In some embodiments, the protein comprises an antibody Fc domain or a portion thereof that binds CD16.

In some embodiments, the first antigen-binding site that binds NKG2D is a Fab fragment, and the second antigen-binding site that binds CEACAM5 is an scFv. In some embodiments, the first antigen-binding site that binds NKG2D is an scFv, and the second antigen-binding site that binds CEACAM5 is a Fab fragment.

In some embodiments, the protein comprising: (i) a first antigen-binding site that binds NKG2D, (ii) a second antigen-binding site that binds CEACAM5, and (iii) a third antigen-binding site, or an antibody Fc domain or a portion thereof, in each case that binds CD16, further comprises an additional antigen-binding site that binds CEACAM5. In some embodiments, the first antigen-binding site that binds NKG2D is an scFv, and the second and the additional antigen-binding sites that bind CEACAM5 are each a Fab fragment. In some embodiments, the first antigen-binding site that binds NKG2D is an scFv, and the second and the additional antigen-binding sites that bind CEACAM5 are each an scFv.

In some embodiments, the scFv that binds CEACAM5 and/or the scFv that binds NKG2D comprise a heavy chain variable domain and a light chain variable domain.

In some embodiments, the scFv is linked to an antibody Fc domain or a portion thereof that binds CD16, via a hinge comprising Ala-Ser or Gly-Ser. In some embodiments, the hinge further comprises amino acid sequence Thr-Lys-Gly. In specific embodiments, the Thr-Lys-Gly is N-terminal or C-terminal to the Ala-Ser or Gly-Ser.

In some embodiments, the heavy chain variable domain of the scFv forms a disulfide bridge with the light chain variable domain of the scFv. In some embodiments, the disulfide bridge is formed between C44 of the heavy chain variable domain and C100 of the light chain variable domain, numbered under the Kabat numbering scheme.

In some embodiments, the heavy chain variable domain of the scFv is linked to the light chain variable domain of the scFv via a flexible linker. In some embodiments, the flexible linker comprises (G4S)4 (SEQ ID NO: 532).

In some embodiments, the heavy chain variable domain of the scFv is positioned at the C-terminus of the light chain variable domain. In some embodiments, the heavy chain variable domain of the scFv is positioned at the N-terminus of the light chain variable domain. In some embodiments, the Fab is not positioned between an antigen-binding site and the antibody Fc domain or the portion thereof.

In some embodiments, the first antigen-binding site that binds NKG2D comprises a VH comprising an amino acid sequence at least 90% identical to a VH sequence selected from Table 1 and a VL comprising an amino acid sequence at least 90% identical to a VL sequence selected from Table 1, wherein the VH sequence and VL sequence selected from Table 1 are from the same clone. In some embodiments, the first antigen-binding site that binds NKG2D comprises a VH comprising CDR1, CDR2, and CDR3 comprising the amino acid sequences of SEQ ID NOs:494 or 495, 496, and 524 or 525, respectively; and a VL comprising CDR1, CDR2, and CDR3 comprising the amino acid sequences of SEQ ID NOs:530, 224, and 499, respectively.

In some embodiments, the first antigen-binding site that binds NKG2D comprises (i) a VH comprising CDR1, CDR2, and CDR3 comprising the amino acid sequences of SEQ ID NOs:494 or 495, 496, and 509 or 510, respectively; and a VL comprising CDR1, CDR2, and CDR3 comprising the amino acid sequences of SEQ ID NOs:530, 224, and 499, respectively. In some embodiments, the first antigen-binding site that binds NKG2D comprises a VH comprising a CDRH1, CDRH2, and CDRH3 comprising the amino acid sequences of SEQ ID NOs: 495, 496, and 510, respectively; and a VL comprising a CDRL1, CDRL2, and CDRL3 comprising the amino acid sequences of SEQ ID NOs:530, 224, and 499, respectively. In some embodiments, the VH of the first antigen-binding site comprises an amino acid sequence at least 90% identical to SEQ ID NO:508, and the VL of the first antigen-binding site comprises an amino acid sequence at least 90% identical to SEQ ID NO:493.

In some embodiments, the VH of the first antigen-binding site comprises the amino acid sequence of SEQ ID NO:508, and the VL of the first antigen-binding site comprises the amino acid sequence of SEQ ID NO:493.

In some embodiments, the antibody Fc domain is a human IgG1 antibody Fc domain. In some embodiments, the antibody Fc domain or the portion thereof comprises an amino acid sequence at least 90% identical to SEQ ID NO:531.

In some embodiments, at least one polypeptide chain of the antibody Fe domain or the portion thereof comprises one or more mutations, relative to SEQ ID NO:531, at one or more positions selected from the group consisting of: Q347, Y349, L351, S354, E356, E357, K360, Q362, S364, T366, L368, K370, N390, K392, T394, D399, S400, D401, F405, Y407, K409, T411, and K439, numbered according to the EU numbering system.

In some embodiments, at least one polypeptide chain of the antibody Fc domain or the portion thereof comprises one or more mutations, relative to SEQ ID NO:531, selected from the group consisting of: Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T3661, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

In some embodiments, one polypeptide chain of the antibody Fc domain or the portion thereof comprises one or more mutations, relative to SEQ ID NO:531, at one or more positions selected from the group consisting of: Q347, Y349, L351, S354, E356, E357, K360, Q362, S364, T366, L368, K370, K392, T394, D399, S400, D401, F405, Y407, K409, T411 and K439, and the other polypeptide chain of the Fc domain or the portion thereof comprises one or more mutations, relative to SEQ ID NO:531, at one or more positions selected from the group consisting of: Q347, Y349, L351, S354, E356, E357, S364, T366, L368, K370, N390, K392, T394, D399, D401, F405, Y407, K409, T411, and K439, numbered according to the EU numbering system.

In some embodiments, one polypeptide chain of the antibody Fc domain or the portion thereof comprises K360E and K409W substitutions relative to SEQ ID NO:531 and the other polypeptide chain of the antibody Fc domain or the portion thereof comprises Q347R, D399V and F405T substitutions relative to SEQ ID NO:531, numbered according to the EU numbering system. In some embodiments, one polypeptide chain of the antibody Fc domain or the portion thereof comprises a Y349C substitution relative to SEQ ID NO:531, and the other polypeptide chain of the antibody Fc domain or the portion thereof comprises an S354C substitution relative to SEQ ID NO:531, numbered according to the EU numbering system.

Another aspect of the present disclosure provides a protein comprising: (a) a first polypeptide comprising the amino acid sequence of SEQ ID NO:549; (b) a second polypeptide comprising the amino acid sequence of SEQ ID NO:550; and (c) a third polypeptide comprising an amino acid sequence selected from the group consisting of: SEQ ID NOs:702, 706, 709, and 713.

Another aspect of the present disclosure provides an isolated nucleic acid molecule, or a plurality of isolated nucleic acid molecules, encoding any of the disclosed proteins.

Another aspect of the present disclosure provides an expression vector comprising an isolated nucleic acid molecule, or a plurality of isolated nucleic molecules, encoding any of the disclosed proteins.

Another aspect of the present disclosure provides a plurality of expression vectors comprising the plurality of isolated nucleic acid molecules.

Another aspect of the present disclosure provides a host cell comprising one or more expression vectors. In some embodiments, the host cell comprises a plurality of expression vectors. In some embodiments, the host cell is a Chinese hamster ovary (CHO) cell.

Another aspect of the present disclosure provides a method of producing a protein comprising: (a) a first antigen-binding site that binds NKG2D; (b) a second antigen-binding site that binds CEACAM5; and (c) a third antigen-binding site, or an antibody Fc domain or a portion thereof, that binds CD16; wherein the method comprises: (i) providing a host cell of the disclosure; (ii) cultivating the host cell in a medium under conditions suitable for expressing the protein; and (iii) isolating the protein from the medium.

Another aspect of the present disclosure provides a method of producing a protein comprising a first, second, and third polypeptide, wherein the method comprises: (a) providing one or more host cell, wherein the one or more host cell comprises an expression vector, or a plurality of expression vectors, comprising: (i) a first isolated nucleic acid molecule encoding the first polypeptide comprising an amino acid sequence of SEQ ID NO:549; (ii) a second isolated nucleic acid molecule encoding the second polypeptide comprising an amino acid sequence of SEQ ID NO:550; and (iii) a third nucleic acid molecule encoding the third polypeptide comprising an amino acid sequence selected from the group consisting of: SEQ ID NOs:702, 706, 709, and 713; (b) culturing the one or more cell in a culture medium under conditions suitable for expressing the first, second, and third polypeptide; (c) recovering the polypeptides from host cell and/or culture medium; and (d) purifying the recovered polypeptide under conditions that thereby produce the protein.

Another aspect of the present disclosure provides a pharmaceutical composition comprising a protein as described herein and a pharmaceutically acceptable carrier.

Another aspect of the present disclosure provides a method of enhancing tumor cell death, the method comprising exposing the tumor cell and a natural killer cell to an effective amount of the protein as described herein or the pharmaceutical composition as described herein.

Another aspect of the present disclosure provides a method of treating cancer, the method comprising administering an effective amount of the protein as described herein or the pharmaceutical composition as described herein to a patient in need thereof.

Another aspect of the present disclosure provides a use of protein in the manufacture of a medicament for the treatment of cancer in a human subject, wherein the protein comprises: (a) a first polypeptide comprising the amino acid sequence of SEQ ID NO:549; (b) a second polypeptide comprising the amino acid sequence of SEQ ID NO: 550; and (c) a third polypeptide comprising an amino acid sequence selected from the group consisting of: SEQ ID NOs: 702, 706, 709, and 713.

In some embodiments, the cancer is selected from the group consisting of: gastrointestinal cancer, colorectal cancer, pancreatic cancer, non-small cell lung cancer, and esophageal cancer. In some embodiments, the cancer expresses CEACAM5.

The invention also provides binding proteins that bind to CEACAM5. The binding proteins comprise an antigen-binding site as disclosed herein. In some aspects, the binding proteins are antibodies or antigen binding fragments thereof having an antigen-binding site as disclosed herein. In other aspects, the antigen-binding site is an antigen binding fragment of an antibody. The present invention also relates to nucleic acids encoding the binding proteins, the methods for making the binding proteins, and the use of the binding proteins in the treatment of disease.

In some embodiments, the antigen-binding site comprises a VH comprising a CDRH1, CDRH2, and CDRH3, and a VL comprising a CDRL1, CDRL2, and CDRL3, wherein: (i) CDRH1 comprises an amino acid sequence selected from the group consisting of: SEQ ID NOs: 3 and 102; (ii) CDRH2 comprises an amino acid sequence selected from the group consisting of: SEQ ID NOs: 37, 104, and 718; (iii) CDRH3 comprises an amino acid sequence selected from the group consisting of: SEQ ID NOs: 6, 38, and 105; (iv) CDRL1 comprises an amino acid sequence selected from the group consisting of: SEQ ID NOs: 7, 40, and 107; (v) CDRL2 comprises an amino acid sequence selected from the group consisting of: SEQ ID NOs: 8, 41, and 108; and (vi) CDRL3 comprises an amino acid sequence selected from the group consisting of: SEQ ID NOs: 9, 42, and 109.

In some embodiments, the CDRH1, CDRH2, and CDRH3 are: (i) SEQ ID NOs: 3, 37, and 38, respectively; (ii) SEQ ID NOs: 3, 718, and 6, respectively; or (iii) SEQ ID NOs: 102, 104, and 105, respectively; and the CDRL1, CDRL2, and CDRL3 of the second antigen-binding site are: (iv) SEQ ID NOs: 7, 8, and 9, respectively; (v) SEQ ID NOs: 40, 41, and 42, respectively; or (vi) SEQ ID NOs: 107, 108, and 109, respectively.

In some embodiments, the CDRH1, CDRH2, CDRH3, CDRL1, CDRL2, and CDRL3 are: (i) SEQ ID NOs: 3, 37, 38, 40, 41, and 42, respectively; (ii) SEQ ID NOs: 3, 718, 6, 7, 8, and 9, respectively; or (iii) SEQ ID NOs: 102, 104, 105, 107, 108, and 109, respectively.

In some embodiments, the VH comprises an amino acid sequence selected from the group consisting of: SEQ ID NOs: 704, 708, 711, and 715; and the VL comprises an amino acid sequence selected from the group consisting of: SEQ ID NOs: 591, 705, 712, and 716.

In some embodiments, the VH and VL are: (i) SEQ ID NOs: 704 and 705, respectively; (ii) SEQ ID NOs: 708 and 591, respectively; (iii) SEQ ID NOs: 711 and 712, respectively; or (iv) SEQ ID NOs: 715 and 716, respectively.

In some embodiments, the antigen-binding site is a Fab fragment or an scFv. In some embodiments, the antigen-binding site is an scFv comprising an amino acid sequence selected from the group consisting of: SEQ ID NOs:703, 707, 710, and 714.

In some embodiments, the antigen-binding site binds a human CEACAM5 variant comprising the amino acid sequence of SEQ ID NO:391.

In some embodiments, the protein comprises an antibody Fc domain or a portion thereof that binds CD16. In some embodiments, the antibody Fc domain or a portion thereof that binds CD16 is linked to the antigen-binding site via a hinge comprising Ala-Ser or Gly-Ser. In some embodiments, the hinge further comprises an amino acid sequence Thr-Lys-Gly.

In some embodiments, the VH domain of the scFv forms a disulfide bridge with the VL domain of the scFv. In some embodiments, the disulfide bridge is formed between C44 of the VH and C100 of the VL, numbered under the Kabat numbering scheme.

In some embodiments, the VH of the scFv is linked to the VL of the scFv via a flexible linker. In some embodiments, the flexible linker comprises (G4S)4 (SEQ ID NO: 532).

In some embodiments, the VH of the scFv is positioned at the C-terminus of the VL. In some embodiments, the VH of the scFv is positioned at the N-terminus of the VL.

In some embodiments, the antibody Fc domain is a human IgG1 antibody Fc domain. In some embodiments, the antibody Fc domain or the portion thereof comprises an amino acid sequence at least 90% identical to SEQ ID NO:531.

In some embodiments, at least one polypeptide chain of the antibody Fc domain or the portion thereof comprises one or more mutations, relative to SEQ ID NO:531, at one or more positions selected from the group consisting of: Q347, Y349, L351, S354, E356, E357, K360, Q362, S364, T366, L368, K370, N390, K392, T394, D399, 5400, D401, F405, Y407, K409, T411, and K439, numbered according to the EU numbering system.

In some embodiments, at least one polypeptide chain of the antibody Fc domain or the portion thereof comprises one or more mutations, relative to SEQ ID NO:531, selected from the group consisting of: Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T3661, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

In some embodiments, one polypeptide chain of the antibody Fc domain or the portion thereof comprises one or more mutations, relative to SEQ ID NO:531, at one or more positions selected from the group consisting of: Q347, Y349, L351, S354, E356, E357, K360, Q362, S364, T366, L368, K370, K392, T394, D399, S400, D401, F405, Y407, K409, T411 and K439; and the other polypeptide chain of the antibody Fc domain or the portion thereof comprises one or more mutations, relative to SEQ ID NO:531, at one or more positions selected from the group consisting of: Q347, Y349, L351, S354, E356, E357, S364, T366, L368, K370, N390, K392, T394, D399, D401, F405, Y407, K409, T411, and K439, numbered according to the EU numbering system.

In some embodiments, one polypeptide chain of the antibody Fc domain or the portion thereof comprises K360E and K409W substitutions relative to SEQ ID NO:531; and the other polypeptide chain of the antibody Fc domain or the portion thereof comprises Q347R, D399V and F405T substitutions relative to SEQ ID NO:531, numbered according to the EU numbering system.

In some embodiments, one polypeptide chain of the antibody heavy chain constant region comprises a Y349C substitution relative to SEQ ID NO:531; and the other polypeptide chain of the antibody heavy chain constant region comprises an S354C substitution relative to SEQ ID NO:531, numbered according to the EU numbering system.

In some embodiments, the protein further comprising a second antigen-binding site that binds CEACAM5.

Another aspect of the present disclosure provides an isolated nucleic acid molecule encoding any of the disclosed proteins, including but not limited to the binding proteins, antibodies, antigen binding fragments or antigen-binding sites.

Another aspect of the present disclosure provides an expression vector comprising an isolated nucleic acid molecule encoding any of the disclosed proteins.

Another aspect of the present disclosure provides a host cell comprising an expression vector comprising an isolated nucleic acid molecule encoding any of the disclosed proteins. In some embodiments, the host cell is a Chinese hamster ovary (CHO) cell.

Another aspect of the present disclosure provides a method of producing a protein comprising: (a) providing a host cell comprising an expression vector comprising an isolated nucleic acid molecule encoding any of the disclosed proteins; (b) cultivating the host cell in a medium under conditions suitable for expressing the protein; and (c) isolating the protein from the medium.

Another aspect of the present disclosure provides a method of enhancing tumor cell death, the method comprising exposing the tumor cell and a natural killer cell to an effective amount of the protein as described herein or the pharmaceutical composition as described herein.

Another aspect of the present disclosure provides a method of treating cancer, the method comprising administering an effective amount of the protein as described herein or the pharmaceutical composition as described herein to a patient in need thereof.

Another aspect of the present disclosure provides a use of protein as described herein in the manufacture of a medicament for the treatment of cancer in a human subject, wherein the protein comprises: (a) a first polypeptide comprising the amino acid sequence of SEQ ID NO:549; (b) a second polypeptide comprising the amino acid sequence of SEQ ID NO:550; and (c) a third polypeptide comprising an amino acid sequence selected from the group consisting of: SEQ ID NOs: 702, 706, 709, and 713.

In some embodiments, the cancer is selected from the group consisting of: gastrointestinal cancer, colorectal cancer, pancreatic cancer, non-small cell lung cancer, and esophageal cancer. In some embodiments, the cancer expresses CEACAM5.

Various aspects and embodiments of the invention are described in further detail below.

### BRIEF DESCRIPTION OF THE DRAWINGS

FIG. 1 is a representation of a heterodimeric, multi-specific antibody, e.g., a trispecific binding protein (TriNKET). Each arm can represent either the NKG2D-binding domain, or the CEACAM5 binding domain. In some embodiments, the NKG2D binding domain and the CEACAM5 binding domains can share a common light chain.

FIGS. 2A-2E illustrate five exemplary formats of a multi-specific binding protein, e.g., a TriNKET. As shown in FIG. 2A, either the NKG2D-binding domain or the CEACAM5 binding domain can take the scFv format (left arm). An antibody that contains an NKG2D-targeting scFv, a CEACAM5-targeting Fab fragment, and a heterodimerized antibody Fe domain, or portion thereof, targeting CD16 is referred herein as the F3-TriNKET. An antibody that contains a CEACAM5-targeting scFv, a NKG2D-targeting Fab fragment, and a heterodimerized antibody Fe domain, or portion thereof, that binds CD16 is referred herein as the F3′-TriNKET (FIG. 2E). As shown in FIG. 2B, both the NKG2D-binding domain and CEACAM5-binding domain can take the scFv format. FIGS. 2C to 2D are illustrations of an antibody with three antigen-binding sites, including two antigen-binding sites that bind CEACAM5, and the NKG2D-binding site fused to the heterodimerized antibody Fc domain, or portion thereof, that binds CD16. These antibody formats are referred herein as F4-TriNKET. FIG. 2C illustrates that the two CEACAM5-binding sites are in the Fab fragment format, and the NKG2D binding site in the scFv format. FIG. 2D illustrates that the CEACAM5 binding sites are in the scFv format, and the NKG2D binding site is in the scFv format. FIG. 2E represents a TriNKET that contains a tumor-targeting scFv, a NKG2D-targeting Fab fragment, and a heterodimerized antibody Fc domain, or portion thereof, that binds CD16, otherwise referred to herein as a constant region/domain (“CD domain”). The antibody format is referred herein as F3′-TriNKET. In certain exemplary multispecific binding proteins, heterodimerization mutations on the antibody Fc domain, or portion thereof, include K360E and K409W on one polypeptide chain of the Fc domain or portion thereof; and Q347R, D399V and F405T on the opposite polypeptide chain of the Fc domain or portion thereof (shown as a triangular lock-and-key shape in the CD domains). The bold bar between the heavy and the light chain variable domains of the Fab fragments represents a disulfide bond.

FIG. 3 is a representation of a TriNKET in the Triomab form, which is a trifunctional, bispecific antibody that maintains an IgG-like shape. This chimera consists of two half antibodies, each with one light and one heavy chain, that originate from two corresponding antibodies. Triomab form may be a heterodimeric construct containing ½ of rat antibody and ½ of mouse antibody.

FIG. 4 is a representation of a TriNKET in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 5 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab fragment targeting antigen 1. DVD-Ig™ form contains normal Fc.

FIG. 6 is a representation of a TriNKET in the Orthogonal Fab fragment interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc.

FIG. 7 is a representation of a TriNKET in the 2-in-1 Ig format.

FIG. 8 is a representation of a TriNKET in the ES form, which is a heterodimeric construct containing two different Fab fragments binding to target 1 and target 2 fused to the Fc. Heterodimerization is ensured by electrostatic steering mutations in the Fc.

FIG. 9 is a representation of a TriNKET in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 10 is a representation of a TriNKET in the SEED Body form, which is a heterodimer containing 2 Fab fragments binding to target 1 and 2, and an Fc stabilized by heterodimerization mutations.

FIG. 11 is a representation of a TriNKET in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 12 is a representation of a TriNKET in the Cov-X-Body form.

FIGS. 13A-13B are representations of TriNKETs in the κλ-Body forms, which are heterodimeric constructs with two different Fab fragments fused to Fc stabilized by heterodimerization mutations: one Fab fragment targeting antigen 1 contains kappa LC, and the second Fab fragment targeting antigen 2 contains lambda LC. FIG. 13A is an exemplary representation of one form of a κλ-Body; FIG. 13B is an exemplary representation of another κλ-Body.

FIG. 14 is an Oasc-Fab heterodimeric construct that includes Fab fragment binding to target 1 and scFab binding to target 2, both of which are fused to the Fc domain. Heterodimerization is ensured by mutations in the Fc domain.

FIG. 15 is a DuetMab, which is a heterodimeric construct containing two different Fab fragments binding to antigens 1 and 2, and an Fc that is stabilized by heterodimerization mutations. Fab fragments 1 and 2 contain differential S—S bridges that ensure correct light chain and heavy chain pairing.

FIG. 16 is a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, while CL is fused in-line with VH.

FIG. 17 is a Fit-Ig, which is a homodimeric construct where Fab fragment binding to antigen 2 is fused to the N-terminus of HC of Fab fragment that binds to antigen 1. The construct contains wild-type Fc.

FIG. 18 are line graphs demonstrating the binding affinity of NKG2D-binding domains (listed as clones) to human recombinant NKG2D in an ELISA assay.

FIG. 19 are line graphs demonstrating the binding affinity of NKG2D-binding domains (listed as clones) to cynomolgus recombinant NKG2D in an ELISA assay.

FIG. 20 are line graphs demonstrating the binding affinity of NKG2D-binding domains (listed as clones) to mouse recombinant NKG2D in an ELISA assay.

FIG. 21 are bar graphs demonstrating the binding of NKG2D-binding domains (listed as clones) to EL4 cells expressing human NKG2D by flow cytometry showing mean fluorescence intensity (MFI) fold over background (FOB).

FIG. 22 are bar graphs demonstrating the binding of NKG2D-binding domains (listed as clones) to EL4 cells expressing mouse NKG2D by flow cytometry showing mean fluorescence intensity (MFI) fold over background (FOB).

FIG. 23 are line graphs demonstrating specific binding affinity of NKG2D-binding domains (listed as clones) to recombinant human NKG2D-Fc by competing with natural ligand ULBP-6.

FIG. 24 are line graphs demonstrating specific binding affinity of NKG2D-binding domains (listed as clones) to recombinant human NKG2D-Fc by competing with natural ligand MICA.

FIG. 25 are line graphs demonstrating specific binding affinity of NKG2D-binding domains (listed as clones) to recombinant mouse NKG2D-Fc by competing with natural ligand Rae-1 delta.

FIG. 26 are bar graphs showing activation of human NKG2D by NKG2D-binding domains (listed as clones) by quantifying the percentage of TNF-α positive cells, which express human NKG2D-CD3 zeta fusion proteins.

FIG. 27 are bar graphs showing activation of mouse NKG2D by NKG2D-binding domains (listed as clones) by quantifying the percentage of TNF-α positive cells, which express mouse NKG2D-CD3 zeta fusion proteins.

FIG. 28 are bar graphs showing activation of human NK cells by NKG2D-binding domains (listed as clones).

FIG. 29 are bar graphs showing activation of human NK cells by NKG2D-binding domains (listed as clones).

FIG. 30 are bar graphs showing activation of mouse NK cells by NKG2D-binding domains (listed as clones).

FIG. 31 are bar graphs showing activation of mouse NK cells by NKG2D-binding domains (listed as clones).

FIG. 32 are bar graphs showing the cytotoxic effect of NKG2D-binding domains (listed as clones) on tumor cells.

FIG. 33 are bar graphs showing the melting temperature of NKG2D-binding domains (listed as clones) measured by differential scanning fluorimetry.

FIGS. 34A-34C are bar graphs of synergistic activation of NK cells using CD16 and NKG2D-binding. FIG. 34A demonstrates levels of CD107a; FIG. 34B demonstrates levels of IFN-γ; FIG. 34C demonstrates levels of CD107a and IFN-γ. Graphs indicate the mean (n=2)±SD. Data are representative of five independent experiments using five different healthy donors.

FIGS. 35A-35B are Biacore sensograms showing the concomitant binding of CEACAM5, NKG2D, and CD16 target proteins to CEACAM5 TriNKETs. FIG. 35B is an enlargement of FIG. 35A, both of which demonstrate heterotetrameric complex formation.

FIGS. 36A-36E are line graphs of flow cytometry experiments which demonstrate the binding of CEACAM5 TriNKETs to various human and cynomolgus CEACAM family member proteins. FIG. 36A demonstrates that AB0411 and AB0466 bound to human CEACAM1; FIG. 36B demonstrates that AB0411 bound to human CEACAM6; FIG. 36C demonstrates that CEACAM5 TriNKETs did not bind to human CEACAM8; FIG. 36D demonstrates that AB0264 and AB0621 bound to cynomolgus CEACAM5 but AB0466 and AB0411 did not; FIG. 36E demonstrates that CEACAM5 TriNKETs did not bind to cells which lacked expression of CEACAM proteins. Data represent the mean of duplicate wells and error bars represent SD.

FIGS. 37A-37D are line graphs from a DELFIA assay showing that CEACAM5 TriNKETs promoted the lysis of target cancer lines SK-CO-1 (FIG. 37A), LS-147T (FIG. 37B), ZR-75-30 (FIG. 37C), and HPAF-II (FIG. 37D).

FIGS. 38A-38B are line graphs from a DELFIA assay showing that AB0264 promoted the lysis of the target cancer line ZR-75-30 better than AB0755, its corresponding mAb. IL-2 activated NK cells (FIG. 38B) showed more potent killing of ZR-75-30 cancer cells compared to rested NK cells (FIG. 38A). Data points represent mean±SD.

FIGS. 39A-39E are line graphs from a DELFIA assay showing that AB0264 promoted the lysis of target cancer lines MKN-45 (FIG. 39A), SK-CO-1 (FIG. 39B), LS-147T (FIG. 39C), ZR-75-30 (FIG. 39D), and HPAF-II (FIG. 39E) better than its corresponding mAb. Data points represent mean±SD.

FIGS. 40A-40E are line graphs from a DELFIA assay showing that AB0411 promoted the lysis of target cancer lines MKN-45 (FIG. 40A), SK-CO-1 (FIG. 40B), LS-147T (FIG. 40C), ZR-75-30 (FIG. 40D), and HPAF-II (FIG. 40E) better than its corresponding mAb. Data points represent mean±SD.

FIG. 41 is a line graph from a DELFIA assay showing that NK-mediated killing of target cells is dependent upon the binding of TriNKETs to CD16, NKG2D, and CEACAM5. Data points represent mean±SD.

FIGS. 42A-42D are line graphs from IFNγ and CD107a activation assays. FIG. 42A demonstrates induction of IFNγ secretion by primary NK cells following co-engagement with CEACAM5 TriNKETs and SK-CO-1 target cells; FIG. 42B demonstrates induction of IFNγ production and CD107a degranulation by primary NK cells following co-engagement with CEACAM5 TriNKETs and MKN-45 target cells; TriNKETs enhanced the degranulation of CD8+NK cells within cynomolgus PBMCs following co-engagement with CEACAM5 TriNKETs and MKN-45 (FIG. 42C) or SK-CO-1 (FIG. 42D) cells. Data are representative of the results of experiments with PBMCs from three animals. Data represent mean±SD.

FIG. 43A is a graph of flow cytometry experiments demonstrating the expression of CEACAM5 protein on cancer cell line SK-CO-1 and patient-derived primary non-small cell lung cancer tumor organoid lines 10910 and 3222. DELFIA assays demonstrated that CEACAM5 TriNKETs promoted the lysis of patient-derived primary non-small lung cancer tumor organoid lines 3222 (FIG. 43B) and 10910 (FIG. 43C).

FIG. 44A is a schematic diagram describing a method of generating activated CD8⁺ T cells. FIG. 44B is a line graph from a DELFIA assay showing that enhancement of pre-activated CD8⁺ T cell-mediated lysis of target cells is dependent upon the binding of TriNKETs to NKG2D and CEACAM5. Max-lysis values represent the mean of three donors±SD.

FIG. 45 are Kaplan-Meier curves demonstrating the percentage of hCEACAM5 transgenic mice with subcutaneous B16F10-hCEACAM5 tumors remaining over time in each mouse IgG2a surrogate for AB0621 (mAB0621) treatment group.

FIGS. 46A-46E are individual curves of tumor volumes for B16F10-hCEACAM5 tumor-bearing mice in a hCEACAM5 transgenic model after administration of isotype control at 15 mg/kg (FIG. 46A) or mAB0621 at 15 mg/kg (FIG. 46B), 5 mg/kg (FIG. 46C), 1.5 mg/kg (FIG. 46D), or 0.5 mg/kg (FIG. 46E) through Day 26.

FIG. 47 are Kaplan-Meier curves demonstrating the percentage of animals remaining over time in each group singly or doubly treated with mAB0621 and/or an anti-PD1 antibody.

FIGS. 48A-48D are the individual B16F10-hCEACAM5 tumor volumes measured for each animal in the isotype control treatment group (FIG. 48A), the anti-PD1 treatment group (FIG. 48B), the mAB0621 treatment group (FIG. 48C), or the mAB0621+anti-PD-1 treatment group (FIG. 48D).

FIGS. 49A-49B are line graphs showing the average serum concentration over time for free and total AB0264 (FIG. 49A) and AB0411 (FIG. 49B) in cynomolgus monkey.

FIGS. 50A-50C are line graphs showing the average serum concentration over time for free and total AB0621 (FIG. 50A), AB0411 (FIG. 50B), and AB0466 (FIG. 50C) in B6.Cg-Tg(hCEACAM5)2682Wzm/Ieg transgenic mice.

### DETAILED DESCRIPTION

The invention provides multi-specific binding proteins that bind the NKG2D receptor and CD16 on natural killer cells, and tumor-associated antigen CEACAM5. In some embodiments, the multi-specific proteins further include an additional antigen-binding site that binds CEACAM5. The invention also provides pharmaceutical compositions comprising such multi-specific binding proteins, and therapeutic methods using such multi-specific proteins and pharmaceutical compositions, for purposes such as treating autoimmune diseases and cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

The term “plurality” as used herein means more than one.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “Fc domain” or “Fc region” as used herein refers to a C-terminal region of an immunoglobulin heavy chain derived from the second and third constant domains. The term includes native sequence Fc regions and variant Fc regions. In some embodiments, the variant Fc region comprises an amino acid sequence that is at least 90% identical (i.e., at least 90%, at least 91%, at least 92%, at least 93%, at least 94%, at least 95%, at least 96%, at least 97%, at least 98%, or at least 99%), to a human native sequence Fc region, e.g., a human IgG1, IgG2, IgG3, or IgG4 Fc region. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The Fe domain as used herein comprises two polypeptide chains that together form the dimeric Fc domain, i.e., each polypeptide comprising a C-terminal constant region of an immunoglobulin heavy chain and capable of self-association. In specific embodiments, a subunit of an IgG Fc domain comprises an IgG CH2 and an IgG CH3 constant domain.

The term “tumor-associated antigen” as used herein means any antigen including but not limited to a protein, glycoprotein, ganglioside, carbohydrate, or lipid that is associated with cancer. Such antigen can be expressed on malignant cells or in the tumor microenvironment such as on tumor-associated blood vessels, extracellular matrix, mesenchymal stroma, or immune infiltrates.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a protein, binding protein, antibody, or antigen-binding fragment of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a pharmaceutically acceptable carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “CEACAM5” (also known as Carcinoembryonic Antigen-Related Cell Adhesion Molecule 5, Meconium Antigen 100, CEA, Carcinoembryonic Antigen, CD66e or CD66e Antigen) refers to the protein of Uniprot Accession No. P06731 and related isoforms.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The invention provides multi-specific binding proteins that bind to the NKG2D receptor and CD16 on natural killer cells, and tumor-associated antigen CEACAM5. The multi-specific binding proteins are useful in the pharmaceutical compositions and therapeutic methods described herein. Binding of the multi-specific binding proteins to the NKG2D receptor and CD16 on a natural killer cell enhances the activity of the natural killer cell toward destruction of tumor cells expressing CEACAM5. Binding of the multi-specific binding proteins to CEACAM5-expressing tumor cells brings these cells into proximity with the natural killer cell, which facilitates direct and indirect destruction of the tumor cells by the natural killer cell. Multi-specific binding proteins that bind NKG2D, CD16, and another target are disclosed in International Application Publication Nos. WO2018148445 and WO2019157366, which are not incorporated herein by reference. Further description of some exemplary multi-specific binding proteins is provided below.

The first component of the multi-specific binding protein is an antigen-binding site that binds to NKG2D receptor-expressing cells, which can include but are not limited to NK cells, γδ T cells, and CD8⁺ αβ T cells. Upon NKG2D binding, the multi-specific binding proteins may block natural ligands, such as ULBP6 and MICA, from binding to NKG2D and activating NK cells.

The second component of the multi-specific binding proteins is an antigen-binding site that binds CEACAM5. CEACAM5-expressing cells may be found, for example, in gastrointestinal cancer, colorectal cancer, pancreatic cancer, non-small cell lung cancer and esophageal cancer.

The third component of the multi-specific binding proteins is an antibody Fc domain or a portion thereof or an antigen-binding site each that binds to cells expressing CD16, including as particular examples an Fc receptor on the surface of leukocytes including natural killer cells, macrophages, neutrophils, eosinophils, mast cells, and follicular dendritic cells.

An additional antigen-binding site of the multi-specific binding proteins may bind CEACAM5. In certain embodiments, the first antigen-binding site that binds NKG2D is an scFv, and the second and the additional antigen-binding sites bind CEACAM5, which are each a Fab fragment. In certain embodiments, the first antigen-binding site that binds NKG2D is an scFv, and the second and the additional antigen-binding sites bind CEACAM5, which are each an scFv. In certain embodiments, the first antigen-binding site that binds NKG2D is a Fab fragment, and the second and the additional antigen-binding sites bind CEACAM5, which are each an scFv. In certain embodiments, the first antigen-binding site that binds NKG2D is a Fab, and the second and the additional antigen-binding sites bind CEACAM5, which are each a Fab fragment.

The antigen-binding sites may each incorporate an antibody heavy chain variable domain and an antibody light chain variable domain (e.g., arranged as in an antibody, or fused together to form an scFv), or one or more of the antigen-binding sites may be a single domain antibody, such as a V_{H}H antibody like a camelid antibody or a V_{NAR} antibody like those found in cartilaginous fish.

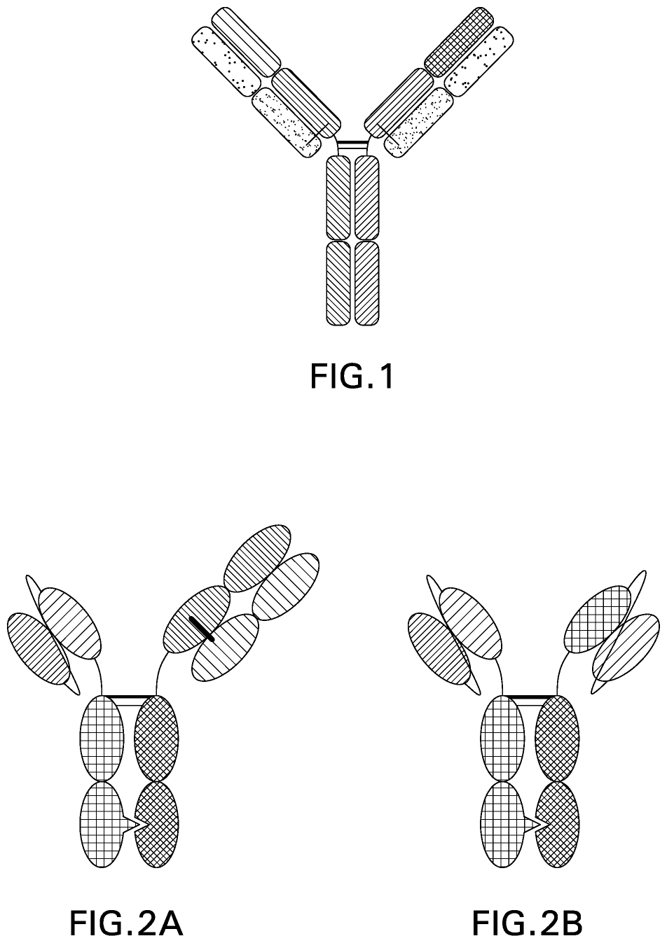

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and optionally a first light chain antibody Fc domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and optionally a second light chain constant domain. The second immunoglobulin light chain, together with the second immunoglobulin heavy chain, forms an antigen-binding site that binds CEACAM5. The first Fc domain and second Fc domain together are able to bind to CD16 (FIG. 1).

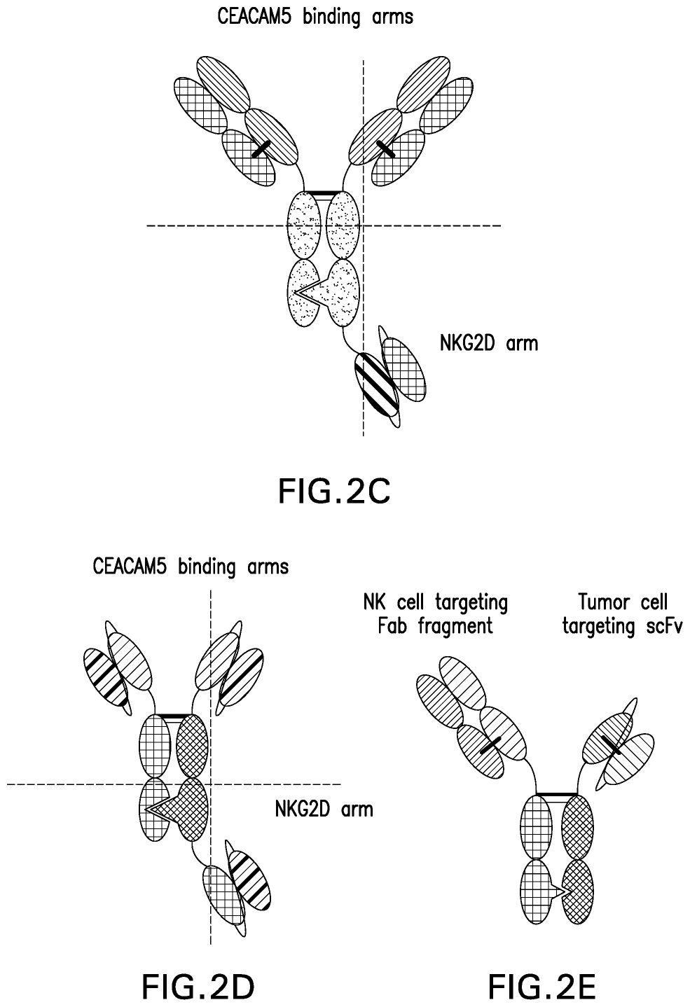

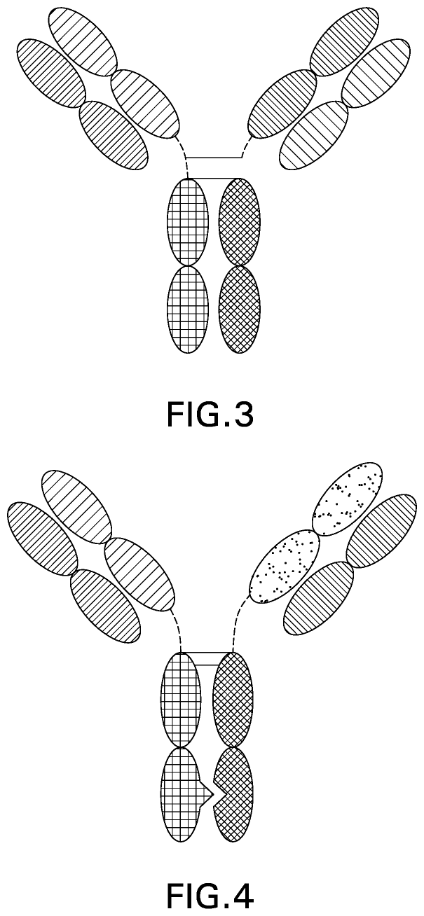

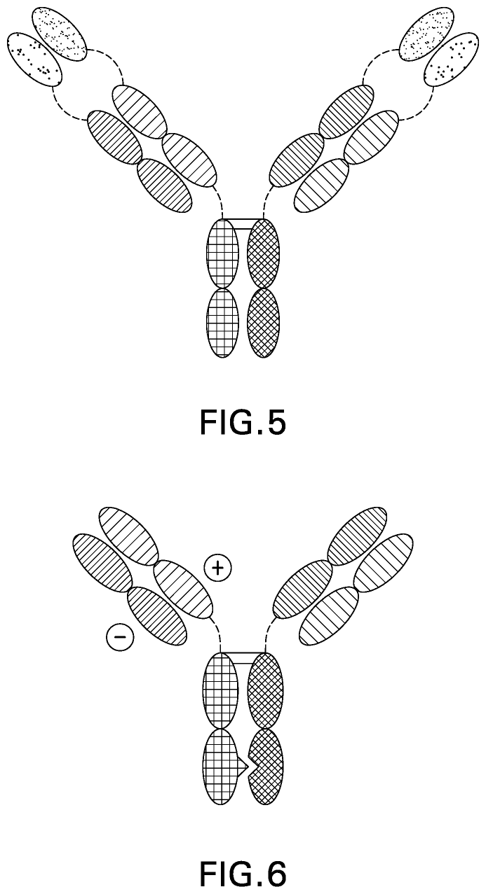

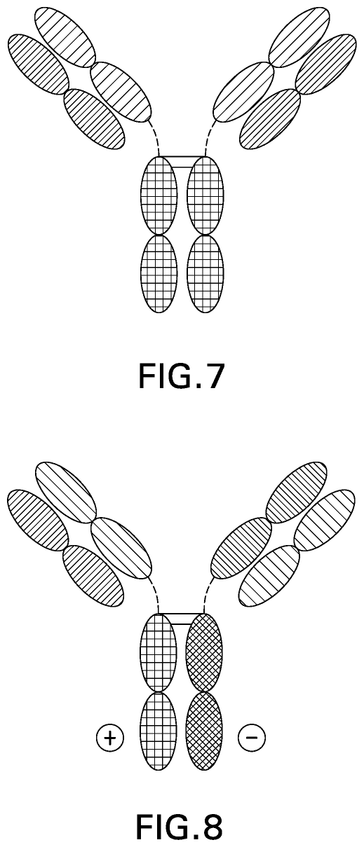

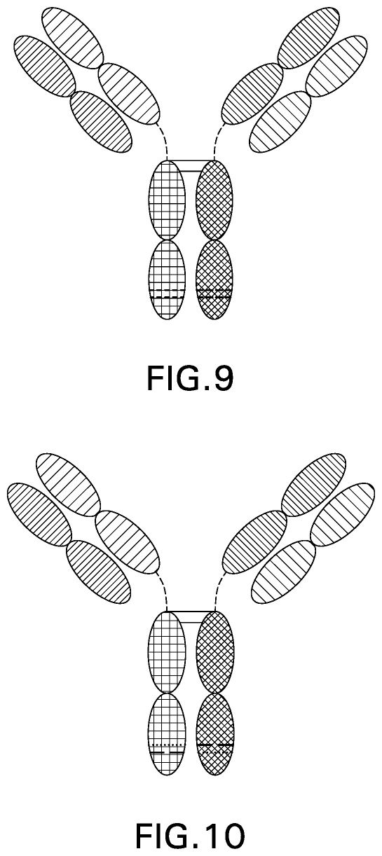

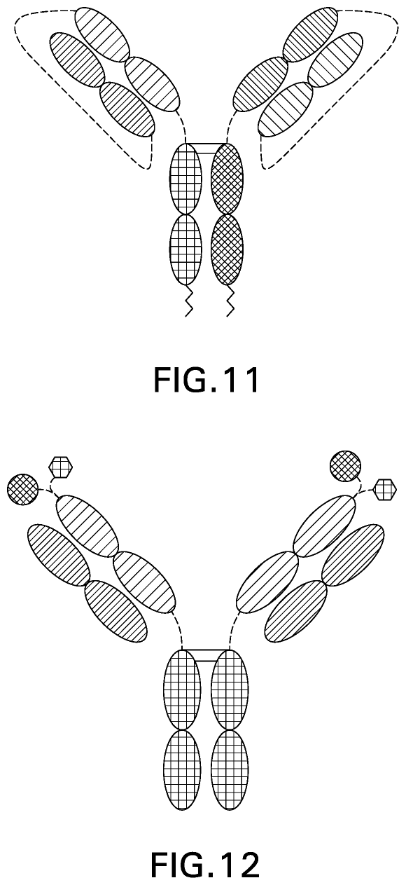

Another exemplary format involves a heterodimeric, multi-specific antibody including a first immunoglobulin heavy chain, a second immunoglobulin heavy chain and an immunoglobulin light chain (FIG. 2A). The first immunoglobulin heavy chain includes a first Fc (hinge-CH2-CH3) domain fused via either a linker or an antibody hinge to an scFv composed of a heavy chain variable domain and light chain variable domain which pair and bind NKG2D, or bind CEACAM5. The second immunoglobulin heavy chain includes a second Fc (hinge-CH2-CH3) domain, a second heavy chain variable domain and a CH1 heavy chain domain. The immunoglobulin light chain includes a light chain variable domain and a light chain constant domain. The second immunoglobulin heavy chain pairs with the immunoglobulin light chain and binds to NKG2D or binds CEACAM5. The first Fe domain and the second Fe domain together are able to bind to CD16 (FIG. 2A).

Another exemplary format involves a heterodimeric, multi-specific antibody including a first immunoglobulin heavy chain, and a second immunoglobulin heavy chain (FIG. 2B). The first immunoglobulin heavy chain includes a first Fc (hinge-CH2-CH3) domain fused via either a linker or an antibody hinge to an scFv composed of a heavy chain variable domain and light chain variable domain which pair and bind NKG2D, or bind CEACAM5. The second immunoglobulin heavy chain includes a second Fc (hinge-CH2-CH3) domain fused via either a linker or an antibody hinge to an scFv composed of a heavy chain variable domain and light chain variable domain which pair and bind NKG2D, or bind CEACAM5. The first Fc domain and the second Fc domain together are able to bind to CD16 (FIG. 2B).

In some embodiments, the scFv described above is linked to the antibody constant domain via a hinge sequence. In some embodiments, the hinge comprises amino acids Ala-Ser or Gly-Ser. In some embodiments, the hinge connects an scFv that binds NKG2D and the antibody heavy chain constant domain comprises amino acids Ala-Ser. In some embodiments, the hinge connects an scFv that binds CEACAM5 and the antibody heavy chain constant domain comprises amino acids Gly-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the scFv described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G₄S)₄ linker (SEQ ID NO: 532). In some embodiments of the scFv, the heavy chain variable domain is positioned at the N-terminus of the light chain variable domain. In some embodiments of the scFv, the heavy chain variable domain is positioned at the C terminus of the light chain variable domain.

The multi-specific binding proteins described herein can further include one or more additional antigen-binding sites. The additional antigen-binding site(s) may be fused to the N-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multispecific binding protein. For example, a multi-specific binding protein includes a first antigen-binding site that binds NKG2D, a second antigen-binding site that binds CEACAM5, an additional antigen-binding site that binds CEACAM5, and an antibody constant region or a portion thereof sufficient to bind CD16 or a fourth antigen-binding site that binds CD16. Any one of these antigen-binding sites can either take the form of a Fab fragment or an scFv, such as an scFv described above.

In some embodiments, the additional antigen-binding site binds a different epitope of CEACAM5 from the second antigen-binding site. In some embodiments, the additional antigen-binding site binds the same epitope as the second antigen-binding site. In some embodiments, the additional antigen-binding site comprises the same heavy chain and light chain CDR sequences as the second antigen-binding site. In some embodiments, the additional antigen-binding site comprises the same heavy chain and light chain variable domain sequences as the second antigen-binding site. In some embodiments, the additional antigen-binding site has the same amino acid sequence(s) as the second antigen-binding site. Exemplary formats are shown in FIG. 2C and FIG. 2D. Accordingly, the multi-specific binding proteins can provide bivalent engagement of CEACAM5. Bivalent engagement of CEACAM5 by the multi-specific proteins can stabilize CEACAM5 on the tumor cell surface and enhance cytotoxicity of NK cells towards the tumor cells. Bivalent engagement of CEACAM5 by the multi-specific proteins can confer stronger binding of the multi-specific proteins to the tumor cells, thereby facilitating stronger cytotoxic response of NK cells towards the tumor cells, especially towards tumor cells expressing a low level of CEACAM5.

The multi-specific binding proteins can take additional formats. In some embodiments, the multi-specific binding protein is in the Triomab form, which is a trifunctional, bispecific antibody that maintains an IgG-like shape. This chimera consists of two half antibodies, each with one light and one heavy chain, that originate from two corresponding antibodies.

In some embodiments, the multi-specific binding protein is the KiH form, which involves the knobs-into-holes (KiHs) technology. The KiH involves engineering CH3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

In some embodiments, the multi-specific binding protein is in the 2-in-1 Ig format. In some embodiments, the multi-specific binding protein is in the ES form, which is a heterodimeric construct containing two different Fab fragments binding to targets 1 and target 2 fused to the Fc. Heterodimerization is ensured by electrostatic steering mutations in the Fc.

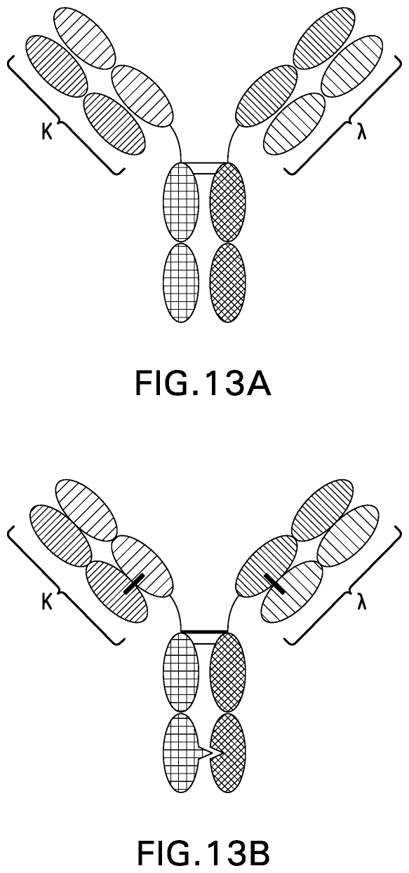

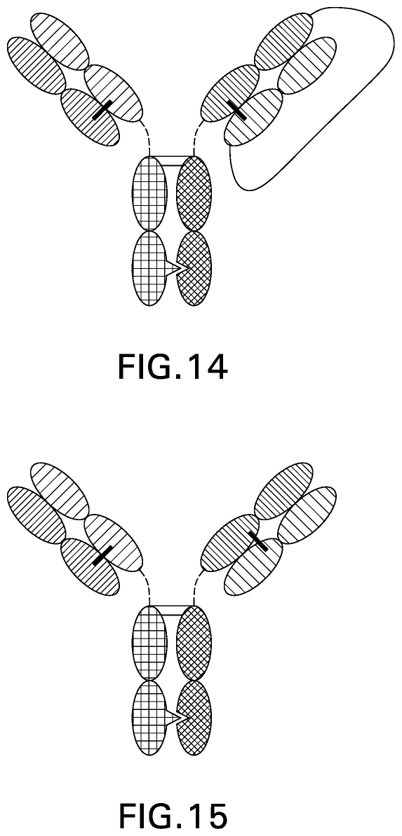

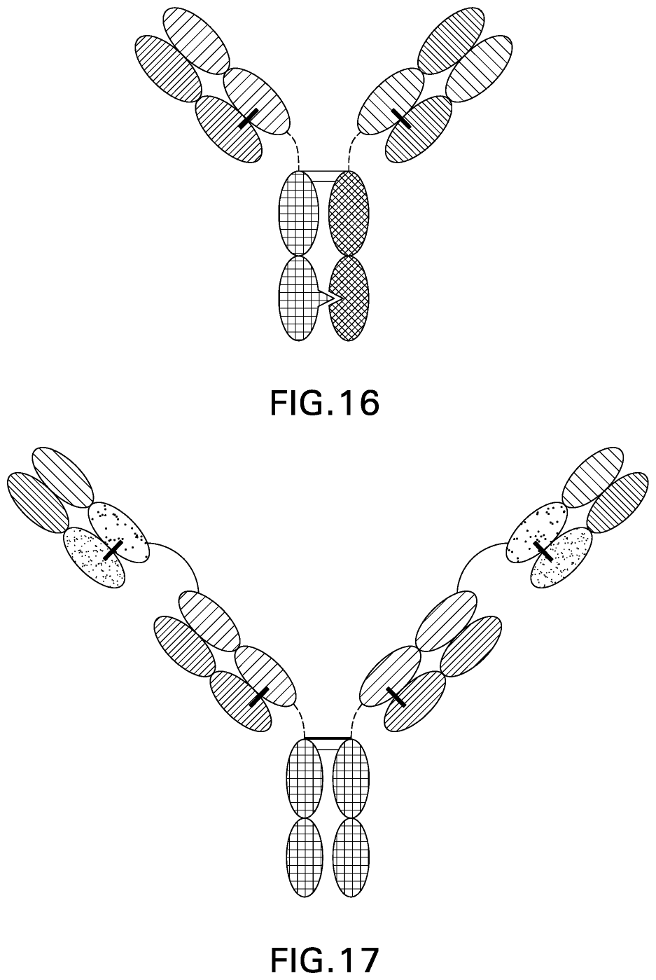

In some embodiments, the multi-specific binding protein is in the κλ-Body form, which is a heterodimeric construct with two different Fab fragments fused to Fc stabilized by heterodimerization mutations: Fab fragment 1 targeting antigen 1 contains kappa LC, while Fab fragment 2 targeting antigen 2 contains lambda LC. FIG. 13A is an exemplary representation of one form of a κλ-Body; FIG. 13B is an exemplary representation of another κλ-Body.

In some embodiments, the multi-specific binding protein is in Fab Arm Exchange form (antibodies that exchange Fab fragment arms by swapping a heavy chain and attached light chain (half-molecule) with a heavy-light chain pair from another molecule, which results in bispecific antibodies).

In some embodiments, the multi-specific binding protein is in the SEED Body form. The strand-exchange engineered domain (SEED) platform was designed to generate asymmetric and bispecific antibody-like molecules, a capability that expands therapeutic applications of natural antibodies. This protein engineering platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in the LuZ-Y form, in which a leucine zipper is used to induce heterodimerization of two different HCs. (Wranik, B J. et al., J. Biol. Chem. (2012), 287:43331-9).

In some embodiments, the multi-specific binding protein is in the Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multi-specific binding protein is in an Oasc-Fab heterodimeric form that includes Fab fragment binding to target 1, and scFab binding to target 2 fused to Fc. Heterodimerization is ensured by mutations in the Fc.

In some embodiments, the multi-specific binding protein is in a DuetMab form, which is a heterodimeric construct containing two different Fab fragments binding to antigens 1 and 2, and Fc stabilized by heterodimerization mutations. Fab fragments 1 and 2 contain differential S—S bridges that ensure correct LC and HC pairing.

In some embodiments, the multi-specific binding protein is in a CrossmAb form, which is a heterodimeric construct with two different Fab fragments binding to targets 1 and 2, fused to Fc stabilized by heterodimerization. CL and CH1 domains and VH and VL domains are switched, e.g., CH1 is fused in-frame with VL, while CL is fused in-frame with VH.

In some embodiments, the multi-specific binding protein is in a Fit-Ig form, which is a homodimeric construct where Fab fragment binding to antigen 2 is fused to the N terminus of HC of Fab fragment that binds to antigen 1. The construct contains wild-type Fc.

Individual components of the multi-specific binding proteins are described in more detail below.

### NKG2D-Binding Site

Upon binding to the NKG2D receptor and CD16 on natural killer cells, and a tumor-associated antigen on cancer cells, the multi-specific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. In some embodiments, the proteins can agonize NK cells in humans and in other species such as cynomolgus monkeys.

Table 1 lists peptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to form an scFv.

The NKG2D binding sites listed in Table 1 can vary in their binding affinity to NKG2D, nevertheless, they all activate human NK cells.

Unless indicated otherwise, the CDR sequences provided in Table 1 are determined under Kabat numbering.

TABLE 1

| | Heavy chain variable region amino | Light chain variable region |
|---|---|---|
| Clones | acid sequence | amino acid sequence |
| ADI- | QVQLQQWGAGLLKPSETLSLTCA | DIQMTQSPSTLSASVGDRVTIT |
| 27705 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYNSYPITFGGGTKVEIK |
| | SS | (SEQ ID NO: 393) |
| | (SEQ ID NO: 392) | |
| | CDR1 (SEQ ID NO: 394)- | |
| | GSFSGYYWS | |
| | CDR2 (SEQ ID NO: 395)- | |
| | EIDHSGSTNYNPSLKS | |
| | CDR3 (SEQ ID NO: 396)- | |
| | ARARGPWSFDP | |
| ADI- | QVQLQQWGAGLLKPSETLSLTCA | EIVLTQSPGTLSLSPGERATLS |
| 27724 | VYGGSFSGYYWSWIRQPPGKGLE | CRASQSVSSSYLAWYQQKPG |
| | WIGEIDHSGSTNYNPSLKSRVTIS | QAPRLLIYGASSRATGIPDRFS |
| | VDTSKNQFSLKLSSVTAADTAVY | GSGSGTDFTLTISRLEPEDFAV |
| | YCARARGPWSFDPWGQGTLVTV | YYCQQYGSSPITFGGGTKVEI |
| | SS | K |
| | (SEQ ID NO: 397) | (SEQ ID NO: 398) |
| ADI- | QVQLQQWGAGLLKPSETLSLTCA | DIQMTQSPSTLSASVGDRVTIT |
| 27740 | VYGGSFSGYYWSWIRQPPGKGLE | CRASQSIGSWLAWYQQKPGK |
| (A40) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYHSFYTFGGGTKVEIK |
| | SS | (SEQ ID NO: 400) |
| | (SEQ ID NO: 399) | |
| ADI- | QVQLQQWGAGLLKPSETLSLTCA | DIQMTQSPSTLSASVGDRVTIT |
| 27741 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQSNSYYTFGGGTKVEIK |
| | SS | (SEQ ID NO: 402) |
| | (SEQ ID NO: 401) | |
| ADI- | QVQLQQWGAGLLKPSETLSLTCA | DIQMTQSPSTLSASVGDRVTIT |
| 27743 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYNSYPTFGGGTKVEIK |
| | SS | (SEQ ID NO: 404) |
| | (SEQ ID NO: 403) | |
| ADI- | QVQLQQWGAGLLKPSETLSLTCA | ELQMTQSPSSLSASVGDRVTIT |
| 28153 | VYGGSFSGYYWSWIRQPPGKGLE | CRTSQSISSYLNWYQQKPGQP |
| | WIGEIDHSGSTNYNPSLKSRVTIS | PKLLIYWASTRESGVPDRFSGS |
| | VDTSKNQFSLKLSSVTAADTAVY | GSGTDFTLTISSLQPEDSATYY |
| | YCARARGPWGFDPWGQGTLVTV | CQQSYDIPYTFGQGTKLEIK |
| | SS | (SEQ ID NO: 406) |
| | (SEQ ID NO: 405) | |
| ADI- | QVQLQQWGAGLLKPSETLSLTCA | DIQMTQSPSTLSASVGDRVTIT |
| 28226 | VYGGSFSGYYWSWIRQPPGKGLE | CRASQSISSWLAWYQQKPGK |
| (C26) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYGSFPITFGGGTKVEIK |
| | SS | (SEQ ID NO: 408) |
| | (SEQ ID NO: 407) | |
| ADI- | QVQLQQWGAGLLKPSETLSLTCA | DIQMTQSPSTLSASVGDRVTIT |
| 28154 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTDFTLTISSLQPDDFATY |
| | YCARARGPWSFDPWGQGTLVTV | YCQQSKEVPWTFGQGTKVEIK |
| | SS | (SEQ ID NO: 410) |
| | (SEQ ID NO: 409) | |
| ADI- | QVQLQQWGAGLLKPSETLSLTCA | DIQMTQSPSTLSASVGDRVTIT |
| 29399 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYNSFPTFGGGTKVEIK |
| | SS | (SEQ ID NO: 412) |
| | (SEQ ID NO: 411) | |
| ADI- | QVQLQQWGAGLLKPSETLSLTCA | DIQMTQSPSTLSASVGDRVTIT |
| 29401 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDIYPTFGGGTKVEIK |
| | SS | (SEQ ID NO: 414) |
| | (SEQ ID NO: 413) | |
| ADI- | QVQLQQWGAGLLKPSETLSLTCA | DIQMTQSPSTLSASVGDRVTIT |
| 29403 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDSYPTFGGGTKVEIK |
| | SS | (SEQ ID NO: 416) |
| | (SEQ ID NO: 415) | |
| ADI- | QVQLQQWGAGLLKPSETLSLTCA | DIQMTQSPSTLSASVGDRVTIT |
| 29405 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYGSFPTFGGGTKVEIK |
| | SS | (SEQ ID NO: 418) |
| | (SEQ ID NO: 417) | |
| ADI- | QVQLQQWGAGLLKPSETLSLTCA | DIQMTQSPSTLSASVGDRVTIT |
| 29407 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYQSFPTFGGGTKVEIK |
| | SS | (SEQ ID NO: 420) |
| | (SEQ ID NO: 419) | |
| ADI- | QVQLQQWGAGLLKPSETLSLTCA | DIQMTQSPSTLSASVGDRVTIT |
| 29419 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYSSFSTFGGGTKVEIK |
| | SS | (SEQ ID NO: 422) |
| | (SEQ ID NO: 421) | |
| ADI- | QVQLQQWGAGLLKPSETLSLTCA | DIQMTQSPSTLSASVGDRVTIT |
| 29421 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYESYSTFGGGTKVEIK |
| | SS | (SEQ ID NO: 424) |
| | (SEQ ID NO: 423) | |
| ADI- | QVQLQQWGAGLLKPSETLSLTCA | DIQMTQSPSTLSASVGDRVTIT |
| 29424 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDSFITFGGGTKVEIK |
| | SS | (SEQ ID NO: 426) |
| | (SEQ ID NO: 425) | |
| ADI- | QVQLQQWGAGLLKPSETLSLTCA | DIQMTQSPSTLSASVGDRVTIT |
| 29425 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYQSYPTFGGGTKVEIK |
| | SS | (SEQ ID NO: 428) |
| | (SEQ ID NO: 427) | |
| ADI- | QVQLQQWGAGLLKPSETLSLTCA | DIQMTQSPSTLSASVGDRVTIT |
| 29426 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYHSFPTFGGGTKVEIK |
| | SS | (SEQ ID NO: 430) |
| | (SEQ ID NO: 429) | |
| ADI- | QVQLQQWGAGLLKPSETLSLTCA | DIQMTQSPSTLSASVGDRVTIT |
| 29429 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYELYSYTFGGGTKVEIK |
| | SS | (SEQ ID NO: 432) |
| | (SEQ ID NO: 431) | |
| ADI- | QVQLQQWGAGLLKPSETLSLTCA | DIQMTQSPSTLSASVGDRVTIT |
| 29447 | VYGGSFSGYYWSWIRQPPGKGLE | CRASQSISSWLAWYQQKPGK |
| (F47) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDTFITFGGGTKVEIK |
| | SS | (SEQ ID NO: 434) |
| | (SEQ ID NO: 433) | |
| ADI- | QVQLVQSGAEVKKPGSSVKVSCK | DIVMTQSPDSLAVSLGERATIN |
| 27727 | ASGGTFSSYAISWVRQAPGQGLE | CKSSQSVLYSSNNKNYLAWY |
| | WMGGIIPIFGTANYAQKFQGRVTI | QQKPGQPPKLLIYWASTRESG |
| | TADESTSTAYMELSSLRSEDTAV | VPDRFSGSGSGTDFTLTISSLQ |
| | YYCARGDSSIRHAYYYYGMDVW | AEDVAVYYCQQYYSTPITFGG |
| | GQGTTVTVSS | GTKVEIK |
| | (SEQ ID NO: 435) | (SEQ ID NO: 436) |
| | CDR1 (SEQ ID NO: 437)- | CDR1 (SEQ ID NO: 440)- |
| | GTFSSYAIS (non-Kabat) or SYAIS | KSSQSVLYSSNNKNYLA |
| | (SEQ ID NO: 438) | CDR2 (SEQ ID NO: 441)- |
| | CDR2 (SEQ ID NO: 439)- | WASTRES |
| | GIIPIFGTANYAQKFQG | CDR3 (SEQ ID NO: 444)- |
| | CDR3 (SEQ ID NO: 442)- | QQYYSTPIT |
| | ARGDSSIRHAYYYYGMDV | |
| | (non-Kabat) or | |
| | GDSSIRHAYYYYGMDV (SEQ ID NO: 443) | |
| ADI- | QLQLQESGPGLVKPSETLSLTCTV | EIVLTQSPATLSLSPGERATLS |
| 29443 | SGGSISSSSYYWGWIRQPPGKGLE | CRASQSVSRYLAWYQQKPGQ |
| (F43) | WIGSIYYSGSTYYNPSLKSRVTISV | APRLLIYDASNRATGIPARFSG |
| | DTSKNQFSLKLSSVTAADTAVYY | SGSGTDFTLTISSLEPEDFAVY |
| | CARGSDRFHPYFDYWGQGTLVT | YCQQFDTWPPTFGGGTKVEIK |
| | VSS | (SEQ ID NO: 454) |
| | (SEQ ID NO: 445) | CDR1 (SEQ ID NO: 451)- |
| | CDR1 (SEQ ID NO: 446)- | RASQSVSRYLA |
| | GSISSSSYYWG (non-Kabat) or | CDR2 (SEQ ID NO: 452)- |
| | SSSYYWG (SEQ ID NO: 447) | DASNRAT |
| | CDR2 (SEQ ID NO: 448)- | CDR3 (SEQ ID NO: 453)- |
| | SIYYSGSTYYNPSLKS | QQFDTWPPT |
| | CDR3 (SEQ ID NO: 449)- | |
| | ARGSDRFHPYFDY(non-Kabat) or | |
| | GSDRFHPYFDY(SEQ ID NO: 450) | |
| ADI- | QVQLQQWGAGLLKPSETLSLTCA | DIQMTQSPSTLSASVGDRVTIT |
| 29404 | VYGGSFSGYYWSWIRQPPGKGLE | CRASQSISSWLAWYQQKPGK |
| (F04) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCEQYDSYPTFGGGTKVEIK |
| | SS (SEQ ID NO: 455) | (SEQ ID NO: 456) |
| ADI- | QVQLVQSGAEVKKPGSSVKVSCK | DIVMTQSPDSLAVSLGERATIN |
| 28200 | ASGGTFSSYAISWVRQAPGQGLE | CESSQSLLNSGNQKNYLTWY |
| | WMGGIIPIFGTANYAQKFQGRVTI | QQKPGQPPKPLIYWASTRESG |
| | TADESTSTAYMELSSLRSEDTAV | VPDRFSGSGSGTDFTLTISSLQ |
| | YYCARRGRKASGSFYYYYGMDV | AEDVAVYYCONDYSYPYTFG |
| | WGQGTTVTVSS | QGTKLEIK |
| | (SEQ ID NO: 457) | (SEQ ID NO: 458) |
| | CDR1 (SEQ ID NO: 437)- | CDR1 (SEQ ID NO: 461)- |
| | GTFSSYAIS | ESSQSLLNSGNQKNYLT |
| | CDR2 (SEQ ID NO: 459)- | CDR2 (SEQ ID NO: 441)- |
| | GIIPIFGTANYAQKFQG | WASTRES |
| | CDR3 (SEQ ID NO: 460)- | CDR3 (SEQ ID NO: 462)- |
| | ARRGRKASGSFYYYYGMDV | QNDYSYPYT |
| ADI- | QVQLVQSGAEVKKPGASVKVSC | EIVMTQSPATLSVSPGERATLS |
| 29379 | KASGYTFTSYYMHWVRQAPGQG | CRASQSVSSNLAWYQQKPGQ |
| (E79) | LEWMGIINPSGGSTSYAQKFQGR | APRLLIYGASTRATGIPARFSG |
| | VTMTRDTSTSTVYMELSSLRSED | SGSGTEFTLTISSLQSEDFAVY |
| | TAVYYCARGAPNYGDTTHDYYY | YCQQYDDWPFTFGGGTKVEI |
| | MDVWGKGTTVTVSS | K |
| | (SEQ ID NO: 463) | (SEQ ID NO: 464) |
| | CDR1 (SEQ ID NO: 465)- | CDR1 (SEQ ID NO: 470)- |
| | YTFTSYYMH (non-Kabat) or | RASQSVSSNLA |
| | SYYMH (SEQ ID NO: 466) | CDR2 (SEQ ID NO: 63)- |
| | CDR2 (SEQ ID NO: 467)- | GASTRAT |
| | IINPSGGSTSYAQKFQG | CDR3 (SEQ ID NO: 472)- |
| | CDR3 (SEQ ID NO: 468)- | QQYDDWPFT |
| | ARGAPNYGDTTHDYYYMDV | |
| | (non-Kabat) or | |
| | GAPNYGDTTHDYYYMDV (SEQ ID NO: 469) | |
| ADI- | QVQLVQSGAEVKKPGASVKVSC | EIVLTQSPGTLSLSPGERATLS |
| 29463 | KASGYTFTGYYMHWVRQAPGQG | CRASQSVSSNLAWYQQKPGQ |
| (F63) | LEWMGWINPNSGGTNYAQKFQG | APRLLIYGASTRATGIPARFSG |
| | RVTMTRDTSISTAYMELSRLRSD | SGSGTEFTLTISSLQSEDFAVY |
| | DTAVYYCARDTGEYYDTDDHGM | YCQQDDYWPPTFGGGTKVEI |
| | DVWGQGTTVTVSS | K |
| | (SEQ ID NO: 473) | (SEQ ID NO: 474) |
| | CDR1 (SEQ ID NO: 475)- | CDR1 (SEQ ID NO: 480)- |
| | YTFTGYYMH (non-Kabat) or | RASQSVSSNLA |
| | GYYMH (SEQ ID NO: 476) | CDR2 (SEQ ID NO: 63)- |
| | CDR2 (SEQ ID NO: 477)- | GASTRAT |
| | WINPNSGGTNYAQKFQG | CDR3 (SEQ ID NO: 481)- |
| | CDR3 (SEQ ID NO: 478)- | QQDDYWPPT |
| | ARDTGEYYDTDDHGMDV (non- | |
| | Kabat) or DTGEYYDTDDHGMDV | |
| | (SEQ ID NO: 479) | |
| ADI- | EVQLLESGGGLVQPGGSLRLSCA | DIQMTQSPSSVSASVGDRVTIT |
| 27744 | ASGFTFSSYAMSWVRQAPGKGLE | CRASQGIDSWLAWYQQKPGK |
| (A44) | WVSAISGSGGSTYYADSVKGRFTI | APKLLIYAASSLQSGVPSRFSG |
| | SRDNSKNTLYLQMNSLRAEDTAV | SGSGTDFTLTISSLQPEDFATY |
| | YYCAKDGGYYDSGAGDYWGQG | YCQQGVSYPRTFGGGTKVEIK |
| | TLVTVSS | (SEQ ID NO: 483) |
| | (SEQ ID NO: 482) | CDR1 (SEQ ID NO: 489)- |
| | CDR1 (SEQ ID NO: 484)- | RASQGIDSWLA |
| | FTFSSYAMS (non-Kabat) or | CDR2 (SEQ ID NO: 224)- |
| | SYAMS (SEQ ID NO: 3) | AASSLQS |
| | CDR2 (SEQ ID NO: 486)- | CDR3 (SEQ ID NO: 491)- |
| | AISGSGGSTYYADSVKG | QQGVSYPRT |
| | CDR3 (SEQ ID NO: 487)- | |
| | AKDGGYYDSGAGDY(non-Kabat) | |
| | or DGGYYDSGAGDY(SEQ ID NO: 488) | |
| ADI- | EVQLVESGGGLVKPGGSLRLSCA | DIQMTQSPSSVSASVGDRVTIT |
| 27749 | ASGFTFSSYSMNWVRQAPGKGLE | CRASQGISSWLAWYQQKPGK |
| (A49) | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPMGAAAGWFDPWGQ | YCQQGVSFPRTFGGGTKVEIK |
| | GTLVTVSS | (SEQ ID NO: 493) |
| | (SEQ ID NO: 492) | CDR1 (SEQ ID NO: 530)- |
| | CDR1 (SEQ ID NO: 494)- | RASQGISSWLA |
| | FTFSSYSMN (non-Kabat) or | CDR2 (SEQ ID NO: 224)- |
| | SYSMN (SEQ ID NO: 495) | AASSLQS |
| | CDR2 (SEQ ID NO: 496)- | CDR3 (SEQ ID NO: 499)- |
| | SISSSSSYIYYADSVKG | QQGVSFPRT |
| | CDR3 (SEQ ID NO: 497)- | |
| | ARGAPMGAAAGWFDP (non- | |
| | Kabat) or GAPMGAAAGWFDP | |
| | (SEQ ID NO: 498) | |
| | scFv (VL-VH) with G44C in VH and G100C in VL, linker italicized: | |
| | DIQMTQSPSSVSASVGDRVTITCRASQGISSWLAWYQQKPGKAPKL | |
| | LIYAASSLQSGVPSRFSGSGSGTDFTLTISSLQPEDFATYYCQQGVSF | |
| | PRTFGCGTKVEIKGGGGSGGGGSGGGGSGGGGSEVQLVESGGGLV | |
| | KPGGSLRLSCAASGFTFSSYSMNWVRQAPGKCLEWVSSISSSSSYIY | |
| | YADSVKGRFTISRDNAKNSLYLQMNSLRAEDTAVYYCARGAPMGA | |
| | AAGWFDPWGQGTLVTVSS (SEQ ID NO: 500) | |
| ADI- | QVQLVQSGAEVKKPGASVKVSC | EIVLTQSPATLSLSPGERATLS |
| 29378 | KASGYTFTSYYMHWVRQAPGQG | CRASQSVSSYLAWYQQKPGQ |
| (E78) | LEWMGIINPSGGSTSYAQKFQGR | APRLLIYDASNRATGIPARFSG |
| | VTMTRDTSTSTVYMELSSLRSED | SGSGTDFTLTISSLEPEDFAVY |
| | TAVYYCAREGAGFAYGMDYYY | YCQQSDNWPFTFGGGTKVEIK |
| | MDVWGKGTTVTVSS | (SEQ ID NO: 502) |
| | (SEQ ID NO: 501) | CDR1 (SEQ ID NO: 506)- |
| | CDR1 (SEQ ID NO: 465)- | RASQSVSSYLA |
| | YTFTSYYMH (non-Kabat) or | CDR2 (SEQ ID NO: 452)- |
| | SYYMH (SEQ ID NO: 466) | DASNRAT |
| | CDR2 (SEQ ID NO: 503)- | CDR3 (SEQ ID NO: 507)- |
| | IINPSGGSTSYAQKFQG | QQSDNWPFT |
| | CDR3 (SEQ ID NO: 504)- | |
| | AREGAGFAYGMDYYYMDV (non- | |
| | Kabat) or | |
| | EGAGFAYGMDYYYMDV (SEQ ID NO: 505) | |
| A49MI | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPIGAAAGWFDPWGQG | YCQQGVSFPRTFGGGTKVEIK |
| | TLVTVSS (SEQ ID NO: 508) | (SEQ ID NO: 493) |
| | CDR1: FTFSSYSMN (SEQ ID | CDR1 (SEQ ID NO: 530)- |
| | NO: 494) (non-Kabat) or SYSMN | RASQGISSWLA |
| | (SEQ ID NO: 495) | CDR2 (SEQ ID NO: 224)- |
| | CDR2: SISSSSSYIYYADSVKG | AASSLQS |
| | (SEQ ID NO: 496) | CDR3 (SEQ ID NO: 499)- |
| | CDR3: (non-Kabat) | QQGVSFPRT |
| | ARGAPIGAAAGWFDP (SEQ ID | |
| | NO: 509) or GAPIGAAAGWFDP | |
| | (SEQ ID NO: 510) | |
| A49MQ | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPQGAAAGWFDPWGQ | YCQQGVSFPRTFGGGTKVEIK |
| | GTLVTVSS | (SEQ ID NO: 493) |
| | (SEQ ID NO: 511) | CDR1 (SEQ ID NO: 530)- |
| | CDR1: FTFSSYSMN (SEQ ID | RASQGISSWLA |
| | NO: 494) (non-Kabat) or SYSMN | CDR2 (SEQ ID NO: 224)- |
| | (SEQ ID NO: 495) | AASSLQS |
| | CDR2: SISSSSSYIYYADSVKG | CDR3 (SEQ ID NO: 499)- |
| | (SEQ ID NO: 496) | QQGVSFPRT |
| | CDR3 (non-Kabat) (SEQ ID NO: 512) | |
| | -ARGAPQGAAAGWFDP or CDR3 | |
| | (SEQ ID NO: 513)- | |
| | GAPQGAAAGWFDP | |
| A49ML | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPLGAAAGWFDPWGQ | YCQQGVSFPRTFGGGTKVEIK |
| | GTLVTVSS | (SEQ ID NO: 493) |
| | (SEQ ID NO: 514) | CDR1 (SEQ ID NO: 530)- |
| | CDR1: FTFSSYSMN (SEQ ID | RASQGISSWLA |
| | NO: 494) (non-Kabat) or SYSMN | CDR2 (SEQ ID NO: 224)- |
| | (SEQ ID NO: 495) | AASSLQS |
| | CDR2: SISSSSSYIYYADSVKG | CDR3 (SEQ ID NO: 499)- |
| | (SEQ ID NO: 496) | QQGVSFPRT |
| | CDR3 (non-Kabat) (SEQ ID NO: 515) | |
| | -ARGAPLGAAAGWFDP or CDR3 | |
| | (SEQ ID NO: 516)- | |
| | GAPLGAAAGWFDP | |
| A49MF | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPFGAAAGWFDPWGQ | YCQQGVSFPRTFGGGTKVEIK |
| | GTLVTVSS | (SEQ ID NO: 493) |
| | (SEQ ID NO: 517) | CDR1 (SEQ ID NO: 530)- |
| | CDR1: FTFSSYSMN (SEQ ID NO: 494) | RASQGISSWLA |
| | (non-Kabat) or SYSMN | CDR2 (SEQ ID NO: 224)- |
| | (SEQ ID NO: 495) | AASSLOS |
| | CDR2: SISSSSSYIYYADSVKG | CDR3 (SEQ ID NO: 499)- |
| | (SEQ ID NO: 496) | QQGVSFPRT |
| | CDR3 (non-Kabat) (SEQ ID NO: 518) | |
| | -ARGAPFGAAAGWFDP or CDR3 | |
| | (SEQ ID NO: 519)- | |
| | GAPFGAAAGWFDP | |
| A49MV | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPVGAAAGWFDPWGQ | YCQQGVSFPRTFGGGTKVEIK |
| | GTLVTVSS | (SEQ ID NO: 493) |
| | (SEQ ID NO: 520) | |
| | CDR1: FTFSSYSMN (SEQ ID | |
| | NO: 494) (non-Kabat) or SYSMN | CDR1 (SEQ ID NO: 530)- |
| | (SEQ ID NO: 495) | RASQGISSWLA |
| | CDR2: SISSSSSYIYYADSVKG | CDR2 (SEQ ID NO: 224)- |
| | (SEQ ID NO: 496) | AASSLQS |
| | CDR3 (non-Kabat) (SEQ ID NO: 521) | CDR3 (SEQ ID NO: 499)- |
| | -ARGAPVGAAAGWFDP or CDR3 | QQGVSFPRT |
| | (SEQ ID NO: 522)- | |
| | GAPVGAAAGWFDP | |
| A49- | EVQLVESGGGLVKPGGSLRLSCA | DIQMTQSPSSVSASVGDRVTIT |
| consensus | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPXGAAAGWFDPWGQ | YCQQGVSFPRTFGGGTKVEIK |
| | GTLVTVSS, wherein X is M, L, I, V, | (SEQ ID NO: 493) |
| | Q, or F | CDR1 (SEQ ID NO: 530)- |
| | (SEQ ID NO: 523) | RASQGISSWLA |
| | CDR1: FTFSSYSMN (SEQ ID | CDR2 (SEQ ID NO: 224)- |
| | NO: 494) (non-Kabat) or SYSMN | AASSLOS |
| | (SEQ ID NO: 495) | CDR3 (SEQ ID NO: 499)- |
| | CDR2: SISSSSSYIYYADSVKG | QQGVSFPRT |
| | (SEQ ID NO: 496) | |
| | CDR3 (non-Kabat) (SEQ ID NO: 524) | |
| | -ARGAPXGAAAGWFDP or CDR3 | |
| | (SEQ ID NO: 525)- | |
| | GAPXGAAAGWFDP, wherein X is | |
| | M, L, I, V, Q, or F | |
| NKG2D | QVQLVESGGGLVKPGGSLRLSCA | QSALTQPASVSGSPGQSITISCS |
| binder in | ASGFTFSSYGMHWVRQAPGKGL | GSSSNIGNNAVNWYQQLPGK |
| | EWVAFIRYDGSNKYYADSVKGRF | APKLLIYYDDLLPSGVSDRFSG |
| | TISRDNSKNTLYLQMNSLRAEDT | SKSGTSAFLAISGLQSEDEADY |
| | AVYYCAKDRGLGDGTYFDYWG | YCAAWDDSLNGPVFGGGTKL |
| U.S. | QGTTVTVSS (SEQ ID NO: 526) | TVL (SEQ ID NO: 527) |
| Pat. No. | | |
| 9,273,136 | | |
| NKG2D | QVHLQESGPGLVKPSETLSLTCTV | EIVLTQSPGTLSLSPGERATLS |
| binder in | SDDSISSYYWSWIRQPPGKGLEWI | CRASQSVSSSYLAWYQQKPG |
| U.S. | GHISYSGSANYNPSLKSRVTISVD | QAPRLLIYGASSRATGIPDRFS |
| Pat. No. | TSKNQFSLKLSSVTAADTAVYYC | GSGSGTDFTLTISRLEPEDFAV |
| 7,879,985 | ANWDDAFNIWGQGTMVTVSS | YYCQQYGSSPWTFGQGTKVEI |
| | (SEQ ID NO: 528) | K (SEQ ID NO: 529) |

In certain embodiments, the first antigen-binding site that binds NKG2D (e.g., human NKG2D) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the first antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of an antibody disclosed in Table 1. In certain embodiments, the first antigen-binding site comprises the heavy chain CDR1, CDR2, and CDR3 and the light chain CDR1, CDR2, and CDR3 of an antibody disclosed in Table 1.

In certain embodiments, the first antigen-binding site that binds to NKG2D comprises a heavy chain variable domain related to SEQ ID NO:392, such as by having an amino acid sequence at least 90% (e.g., at least 90%, at least 91%, at least 92%, at least 93%, at least 94%, at least 95%, at least 96%, at least 97%, at least 98%, at least 99%, or 100%) identical to SEQ ID NO:392, and/or incorporating amino acid sequences identical to the CDR1 (SEQ ID NO:394), CDR2 (SEQ ID NO:395), and CDR3 (SEQ ID NO:396) sequences of SEQ ID NO:392. The heavy chain variable domain having at least 90% sequence identity to SEQ ID NO:392 can be coupled with a variety of light chain variable domains to form an NKG2D binding site. For example, the first antigen-binding site that incorporates a heavy chain variable domain having at least 90% sequence identity SEQ ID NO:392 can further incorporate a light chain variable domain having at least 90% sequence identity to any one of the sequences selected from the group consisting of: SEQ ID NOs: 393, 398, 400, 402, 404, 408, 410, 412, 414, 416, 418, 420, 422, 424, 426, 428, 430, 432, and 434. For example, the first antigen-binding site incorporates a heavy chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to SEQ ID NO:392 and a light chain variable domain with amino acid sequences at least 90% (e.g., at least 90%, at least 91%, at least 92%, at least 93%, at least 94%, at least 95%, at least 96%, at least 97%, at least 98%, at least 99%, or 100%) identical to any one of the sequences selected from the group consisting of: SEQ ID NOs: 393, 398, 400, 402, 404, 408, 410, 412, 414, 416, 418, 420, 422, 424, 426, 428, 430, 432, and 434.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:435, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:436. In certain embodiments, the VH comprises CDR1, CDR2, and CDR3 comprising the amino acid sequences of SEQ ID NOs: 437 or 438, 439, and 442 or 443, respectively. In certain embodiments, the VL comprises CDR1, CDR2, and CDR3 comprising the amino acid sequences of SEQ ID NOs: 440, 441, and 444, respectively. In certain embodiments, the first antigen-binding site comprises (a) a VH that comprises CDR1, CDR2, and CDR3 comprising the amino acid sequences of SEQ ID NOs: 437 or 438, 439, and 442 or 443, respectively; and (b) a VL that comprises CDR1, CDR2, and CDR3 comprising the amino acid sequences of SEQ ID NOs: 440, 441, and 444, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:445, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:454. In certain embodiments, the VH comprises CDR1, CDR2, and CDR3 comprising the amino acid sequences of SEQ ID NOs: 446 or 447, 448, and 449 or 450, respectively. In certain embodiments, the VL comprises CDR1, CDR2, and CDR3 comprising the amino acid sequences of SEQ ID NOs: 451, 452, and 453, respectively. In certain embodiments, the first antigen-binding site comprises (a) a VH that comprises CDR1, CDR2, and CDR3 comprising the amino acid sequences of SEQ ID NOs: 446 or 447, 448, and 449 or 450, respectively; and (b) a VL that comprises CDR1, CDR2, and CDR3 comprising the amino acid sequences of SEQ ID NOs: 451, 452, and 453, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:455, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:456.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:457, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:458. In certain embodiments, the VH comprises CDR1, CDR2, and CDR3 comprising the amino acid sequences of SEQ ID NOs: 437, 459, and 460, respectively. In certain embodiments, the VL comprises CDR1, CDR2, and CDR3 comprising the amino acid sequences of SEQ ID NOs: 461, 441, and 462, respectively. In certain embodiments, the first antigen-binding site comprises (a) a VH that comprises CDR1, CDR2, and CDR3 comprising the amino acid sequences of SEQ ID NOs: 437, 459, and 460, respectively; and (b) a VL that comprises CDR1, CDR2, and CDR3 comprising the amino acid sequences of SEQ ID NOs: 461, 441, and 462, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:463, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:464. In certain embodiments, the VH comprises CDR1, CDR2, and CDR3 comprising the amino acid sequences of SEQ ID NOs: 465 or 466, 467, and 468 or 469, respectively. In certain embodiments, the VL comprises CDR1, CDR2, and CDR3 comprising the amino acid sequences of SEQ ID NOs: 470, 63, and 472, respectively. In certain embodiments, the first antigen-binding site comprises (a) a VH that comprises CDR1, CDR2, and CDR3 comprising the amino acid sequences of SEQ ID NOs: 465 or 466, 467, and 468 or 469, respectively; and (b) a VL that comprises CDR1, CDR2, and CDR3 comprising the amino acid sequences of SEQ ID NOs: 470, 63, and 472, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:473, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:474. In certain embodiments, the VH comprises CDR1, CDR2, and CDR3 comprising the amino acid sequences of SEQ ID NOs: 475 or 476, 477, and 478 or 479, respectively. In certain embodiments, the VL comprises CDR1, CDR2, and CDR3 comprising the amino acid sequences of SEQ ID NOs: 480, 63, and 481, respectively. In certain embodiments, the first antigen-binding site comprises (a) a VH that comprises CDR1, CDR2, and CDR3 comprising the amino acid sequences of SEQ ID NOs: 475 or 476, 477, and 478 or 479, respectively; and (b) a VL that comprises CDR1, CDR2, and CDR3 comprising the amino acid sequences of SEQ ID NOs: 480, 63, and 481, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:501, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:502. In certain embodiments, the VH comprises CDR1, CDR2, and CDR3 comprising the amino acid sequences of SEQ ID NOs: 465 or 466, 503, and 504 or 505, respectively. In certain embodiments, the VL comprises CDR1, CDR2, and CDR3 comprising the amino acid sequences of SEQ ID NOs: 506, 452, and 507, respectively. In certain embodiments, the first antigen-binding site comprises (a) a VH that comprises CDR1, CDR2, and CDR3 comprising the amino acid sequences of SEQ ID NOs: 465 or 466, 503, and 504 or 505, respectively; and (b) a VL that comprises CDR1, CDR2, and CDR3 comprising the amino acid sequences of SEQ ID NOs: 506, 452, and 507, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:482, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:483. In certain embodiments, the VH comprises CDR1, CDR2, and CDR3 comprising the amino acid sequences of SEQ ID NOs: 484 or 3, 486, and 487 or 488, respectively. In certain embodiments, the VL comprises CDR1, CDR2, and CDR3 comprising the amino acid sequences of SEQ ID NOs: 489, 490, and 491, respectively. In certain embodiments, the first antigen-binding site comprises (a) a VH that comprises CDR1, CDR2, and CDR3 comprising the amino acid sequences of SEQ ID NOs: 484 or 3, 486, and 487 or 488, respectively; and (b) a VL that comprises CDR1, CDR2, and CDR3 comprising the amino acid sequences of SEQ ID NOs: 489, 490, and 491, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:492, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 497 or 498, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 497 or 498 respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:508, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 509 or 510, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 509 or 510, respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:511, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 512 or 513, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 512 or 513, respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:514, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 515 or 516, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 515 or 516, respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:517, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 518 or 519, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 518 or 519, respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:520, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 521 or 522, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 521 or 522, respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:523, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:493. In certain embodiments, the VH comprises CDR1, CDR2, and CDR3 comprising the amino acid sequences of SEQ ID NOs: 494 or 495, 496, and 524 or 525, respectively. In certain embodiments, the VL comprises CDR1, CDR2, and CDR3 comprising the amino acid sequences of SEQ ID NOs: 530, 224, and 499, respectively. In certain embodiments, the first antigen-binding site comprises (a) a VH that comprises CDR1, CDR2, and CDR3 comprising the amino acid sequences of SEQ ID NOs: 494 or 495, 496, and 524 or 525, respectively; and (b) a VL that comprises CDR1, CDR2, and CDR3 comprising the amino acid sequences of SEQ ID NOs: 530, 224, and 499, respectively.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:526, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:527.

In certain embodiments, the first antigen-binding site that binds NKG2D comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:528, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:529.

The multi-specific binding proteins can bind to NKG2D-expressing cells, which include but are not limited to NK cells, γδ T cells and CD8⁺ αβ T cells. Upon NKG2D binding, the multi-specific binding proteins may block natural ligands, such as ULBP6 and MICA, from binding to NKG2D and activating NK cells.

The multi-specific binding proteins binds to cells expressing CD16, an Fc receptor on the surface of leukocytes including natural killer cells, macrophages, neutrophils, eosinophils, mast cells, and follicular dendritic cells. A protein of the present disclosure binds to NKG2D with an affinity of K_{D} of 2 nM to 120 nM, e.g., 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, or about 8 nM to about 10 nM. In some embodiments, NKG2D-binding sites bind to NKG2D with a K_{D} of 10 to 62 nM.

### CEACAM5-Binding Site

The CEACAM5-binding site of the multi-specific binding protein disclosed herein comprises a heavy chain variable domain and a light chain variable domain. Table 2 lists some exemplary sequences of heavy chain variable domains and light chain van able domains that, in combination, can bind to CEACAM5. CDR sequences are identified under Chothia and Kabat numbering as indicated. Cysteine mutations for disulfide bond formation are underlined. The scFv sequences include a (G₄S)₄ linker (SEQ ID NO: 532) (italicized) between the VH and VL.

TABLE 2
Sequences of Exemplary Antigen-Binding Sites that Bind CEACAM5

| | VH | VL |
|---|---|---|
| Cognate Pair | QVQLVQSGGGLVQPGGSLRLSC | DIRMTQSPSTLSASVGDRVTITCW |
| A1 | AASGFTFSSYAMSWVRQAPGK | ASQSISSWLAWYQQKPGKAPKLLI |
| (derived from | CLEWVSAIFNSGGSTYYADSVK | YKASSLESGVPSRFSGSGSGTEFTL |
| PH_420- | GRFTVSRDNSKNTLYLQMNSLR | TISSLQPDDFATYYCQQYNSYSYT |
| CEACAM5) | AEDTALYYCAKDLGGYNYGLF | FGCGTKLEIK (SEQ ID NO: 568) |
| | DYWGQGTLVTVSS (SEQ ID | CDR1: WASQSISSWLA (SEQ ID |
| | NO: 567) | NO: 7) |
| | CDR1: GFTFSSY (SEQ ID NO: 2) | CDR2: KASSLES (SEQ ID NO: 8) |
| | (Chothia) or SYAMS (SEQ ID | CDR3: QQYNSYSYT (SEQ ID NO: 9) |
| | NO: 3)(Kabat) | |
| | CDR2: FNSGGS (SEQ ID NO: 4) | |
| | (Chothia) or | |
| | AIFNSGGSTYYADSVKG (SEQ | |
| | ID NO: 5)(Kabat) | |
| | CDR3: DLGGYNYGLFDY (SEQ | |
| | ID NO: 6) | |
| scFv for | GB1 (VL-VH): | |
| GB1/GB2 | DIRMTQSPSTLSASVGDRVTITCWASQSISSWLAWYQQKPGKAPKLLI | |
| (derived from | YKASSLESGVPSRFSGSGSGTEFTLTISSLQPDDFATYYCQQYNSYSYT | |
| Cognate Pair | FGCGTKLEIKGGGGSGGGGSGGGGSGGGGSQVQLVQSGGGLVQPGGS | |
| A1) | LRLSCAASGFTFSSYAMSWVRQAPGKCLEWVSAIFNSGGSTYYADSV | |
| | KGRFTVSRDNSKNTLYLQMNSLRAEDTALYYCAKDLGGYNYGLFDY | |
| | WGQGTLVTVSS (SEQ ID NO: 569) | |
| | GB2 (VH-VL): | |
| | QVQLVQSGGGLVQPGGSLRLSCAASGFTFSSYAMSWVRQAPGKCLE | |
| | WVSAIFNSGGSTYYADSVKGRFTVSRDNSKNTLYLQMNSLRAEDTAL | |
| | YYCAKDLGGYNYGLFDYWGQGTLVTVSSGGGGSGGGGSGGGGSGG | |
| | GGSDIRMTQSPSTLSASVGDRVTITCWASQSISSWLAWYQQKPGKAPK | |
| | LLIYKASSLESGVPSRFSGSGSGTEFTLTISSLQPDDFATYYCQQYNSYS | |
| | YTFGCGTKLEIK (SEQ ID NO: 586) | |
| Cognate Pair | EVQLVQSGGGLVQPGGSLRLSC | DIRMTQSPSTLSASVGDRVTITCW |
| A2 | AASGFTFSSYAMSWVRQAPGK | ASQSISSWLAWYQQKPGKAPKLLI |
| (VH derived | CLEWVSAIFNSGGSTYYADSVK | YKASSLESGVPSRFSGSGSGTEFTL |
| from | GRFTVSRDNSKNTLYLQMNSLR | TISSLQPDDFATYYCQQYNSYSYT |
| PH_420- | AEDTALYYCAKDLGGYNYGLF | FGCGTKLEIK (SEQ ID NO: 588) |
| CEACAM5_ | DYWGQGTLVTV (SEQ ID | CDR1: WASQSISSWLA (SEQ ID |
| VH_Variant 1 | NO: 587) | NO: 7) |
| (QE) and VL | CDR1: GFTFSSY (SEQ ID NO: 2) | CDR2: KASSLES (SEQ ID NO: 8) |
| derived from | (Chothia) or SYAMS (SEQ ID | CDR3: QQYNSYSYT (SEQ ID NO: 9) |
| PH_420- | NO: 3)(Kabat) | |
| CEACAM5) | CDR2: FNSGGS (SEQ ID NO: 4) | |
| | (Chothia) or | |
| | AIFNSGGSTYYADSVKG (SEQ | |
| | ID NO: 5)(Kabat) | |
| | CDR3: DLGGYNYGLFDY (SEQ | |
| | ID NO: 6) | |
| scFv for GB3/ | GB3 (VH-VL): | |
| GB4 (derived | EVQLVQSGGGLVQPGGSLRLSCAASGFTFSSYAMSWVRQAPGKCLE | |
| from Cognate | WVSAIFNSGGSTYYADSVKGRFTVSRDNSKNTLYLQMNSLRAEDTAL | |
| Pair A2) | YYCAKDLGGYNYGLFDYWGQGTLVTVSSGGGGSGGGGSGGGGSGG | |
| | GGSDIRMTQSPSTLSASVGDRVTITCWASQSISSWLAWYQQKPGKAPK | |
| | LLIYKASSLESGVPSRFSGSGSGTEFTLTISSLQPDDFATYYCQQYNSYS | |
| | YTFGCGTKLEIK (SEQ ID NO: 570) | |
| | GB4 (VL-VH): | |
| | DIRMTQSPSTLSASVGDRVTITCWASQSISSWLAWYQQKPGKAPKLLI | |
| | YKASSLESGVPSRFSGSGSGTEFTLTISSLQPDDFATYYCQQYNSYSYT | |
| | FGCGTKLEIKGGGGSGGGGSGGGGSGGGGSEVQLVQSGGGLVQPGGS | |
| | LRLSCAASGFTFSSYAMSWVRQAPGKCLEWVSAIFNSGGSTYYADSV | |
| | KGRFTVSRDNSKNTLYLQMNSLRAEDTALYYCAKDLGGYNYGLFDY | |
| | WGQGTLVTVSS (SEQ ID NO: 589) | |
| Cognate Pair | QVQLVQSGGGLVQPGGSQRLS | DIQLTQSPATLSVSPGERATLSCRA |
| A3 | CAASGFTFTSYAMSWVRQAPG | SQSVSSSYLAWYQQKPGQAPRLLI |
| (derived from | KCLEWVSAISGTGDSTFYADSV | YGASSRATGIPDRFSGSGSGTDFTL |
| 1078_C04- | KGRFTFSRDNSKNTLYLQMNSL | TISRLEPEDFAVYYCQQYNNWPLT |
| CEACAM5) | RAEDTAVYYCAKDLGWLQYG | FGCGTKVEIK (SEQ ID NO: 591) |
| | LFDYWGQGTLVTVSS (SEQ ID | CDR1: RASQSVSSSYLA (SEQ ID |
| | NO: 590) | NO: 40) |
| | CDR1: GFTFTSY (SEQ ID NO: 35) | CDR2: GASSRAT (SEQ ID NO: 41) |
| | (Chothia) or SYAMS (SEQ ID | CDR3: QQYNNWPLT (SEQ ID |
| | NO: 3)(Kabat) | NO: 42) |
| | CDR2: SGTGDS (SEQ ID NO: 36) | |
| | (Chothia) or | |
| | AISGTGDSTFYADSVKG (SEQ | |
| | ID NO: 37)(Kabat) | |
| | CDR3: DLGWLQYGLFDY (SEQ | |
| | ID NO: 38) | |
| scFv for | GB5 (VH-VL): | |
| GB5/GB6 | QVQLVQSGGGLVQPGGSQRLSCAASGFTFTSYAMSWVRQAPGKCLE | |
| (derived from | WVSAISGTGDSTFYADSVKGRFTFSRDNSKNTLYLQMNSLRAEDTAV | |
| Cognate Pair | YYCAKDLGWLQYGLFDYWGQGTLVTVSSGGGGSGGGGSGGGGSGG | |
| A3) | GGSDIQLTQSPATLSVSPGERATLSCRASQSVSSSYLAWYQQKPGQAP | |
| | RLLIYGASSRATGIPDRFSGSGSGTDFTLTISRLEPEDFAVYYCQQYNN | |
| | WPLTFGCGTKVEIK (SEQ ID NO: 571) | |
| | GB6 (VL-VH) | |
| | DIQLTQSPATLSVSPGERATLSCRASQSVSSSYLAWYQQKPGQAPRLLI | |
| | YGASSRATGIPDRFSGSGSGTDFTLTISRLEPEDFAVYYCQQYNNWPL | |
| | TFGCGTKVEIKGGGSGGGGSGGGGSGGGGSQVQLVQSGGGLVQPGGS | |
| | QRLSCAASGFTFTSYAMSWVRQAPGKCLEWVSAISGTGDSTFYADSV | |
| | KGRFTFSRDNSKNTLYLQMNSLRAEDTAVYYCAKDLGWLQYGLFDY | |
| | WGQGTLVTVSSG (SEQ ID NO: 592) | |
| Cognate Pair | EVQLVQSGGGLVQPGGSQRLSC | DIQLTQSPATLSVSPGERATLSCRA |
| A4 | AASGFTFTSYAMSWVRQAPGK | SQSVSSSYLAWYQQKPGQAPRLLI |
| (derived from | CLEWVSAISGTGDSTFYADSVK | YGASSRATGIPDRFSGSGSGTDFTL |
| 1078_C04 | GRFTFSRDNSKNTLYLQMNSLR | TISRLEPEDFAVYYCQQYNNWPLT |
| CEACAM5_ | AEDTAVYYCAKDLGWLQYGLF | FGCGTKVEIK (SEQ ID NO: 594) |
| VH_ | DYWGQGTLVTVSS (SEQ ID | CDR1: RASQSVSSSYLA (SEQ ID |
| Variant 1 | NO: 593) | NO: 40) |
| (QE)) | CDR1: GFTFTSY (SEQ ID NO: 35) | CDR2: GASSRAT (SEQ ID NO: 41) |
| | (Chothia) or SY AMS (SEQ ID | CDR3: QQYNNWPLT (SEQ ID |
| | NO: 3)(Kabat) | NO: 42) |
| | CDR2: SGTGDS (SEQ ID NO: 36) | |
| | (Chothia) or | |
| | AISGTGDSTFYADSVKG (SEQ | |
| | ID NO: 37)(Kabat) | |
| | CDR3: DLGWLQYGLFDY (SEQ | |
| | ID NO: 38) | |
| scFv for GB7/ | GB7 (VH-VL): | |
| GB8 | EVQLVQSGGGLVQPGGSQRLSCAASGFTFTSYAMSWVRQAPGKCLE | |
| (derived from | WVSAISGTGDSTFYADSVKGRFTFSRDNSKNTLYQMNSLRAEDTAV | |
| Cognate Pair | YYCAKDLGWLQYGLFDYWGQGTLVTVSSGGGGSGGGGSGGGGSGG | |
| A4) | GGSDIQLTQSPATLSVSPGERATLSCRASQSVSSSYLAWYQQKPGQAP | |
| | RLLIYGASSRATGIPDRFSGSGSGTDFTLTISRLEPEDFAVYYCQQYNN | |
| | WPLTFGCGTKVEIK (SEQ ID NO: 572) | |
| | GB8 (VL-VH): | |
| | DIQLTQSPATLSVSPGERATLSCRASQSVSSSYLAWYQQKPGQAPRLLI | |
| | YGASSRATGIPDRFSGSGSGTDFTLTISRLEPEDFAVYYCQQYNNWPL | |
| | TFGCGTKVEIKGGGGSGGGGSGGGGSGGGGSEVQLVQSGGGLVQPGG | |
| | SQRLSCAASGFTFTSYAMSWVRQAPGKCLEWVSAISGTGDSTFYADS | |
| | VKGRFTFSRDNSKNTLYLQMNSLRAEDTAVYYCAKDLGWLQYGLFD | |
| | YWGQGTLVTVSS (SEQ ID NO: 595) | |
| Cognate Pair | QVQLVQSGGGLVQPGGSQRLS | DIQLTQSPATLSVSPGERATLSCRA |
| A5 (derived | CAASGFTFTSYAMSWVRQAPG | SQSVSSSYLAWYQQKPGQAPRLLI |
| from | KCLEWVSAISGTGDSTFYADSV | YGASSRATGIPDRFSGSGSGTDFTL |
| 1078_C04- | KGRFTFSRDNSKNTLYLQMNSL | TISRLEPEDFAVYYCQQYNNWPLT |
| CEACAM5) | RAEDTAVYYCAKDLGWLQYG | FGCGTKVEIK (SEQ ID NO: 597) |
| | LFDYWGQGTLVTVSS(SEQ ID | CDR1: RASQSVSSSYLA (SEQ ID |
| | NO: 596) | NO: 40) |
| | CDR1: GFTFTSY (SEQ ID NO: 35) | CDR2: GASSRAT (SEQ ID NO: 41) |
| | (Chothia) or SYAMS (SEQ ID | CDR3: QQYNNWPLT (SEQ ID |
| | NO: 3)(Kabat) | NO: 42) |
| | CDR2: SGTGDS (SEQ ID NO: 36) | |
| | (Chothia) or | |
| | AISGTGDSTFYADSVKG (SEQ | |
| | ID NO: 37)(Kabat) | |
| | CDR3: DLGWLQYGLFDY (SEQ | |
| | ID NO: 38) | |
| scFv for GB9/ | GB9 (VL-VH): | |
| GB10 | DIQLTQSPATLSVSPGERATLSCRASQSVSSSYLAWYQQKPGQAPRLLI | |
| (derived from | YGASSRATGIPDRFSGSGSGTDFTLTISRLEPEDFAVYYCQQYNNWPL | |
| Cognate Pair | TFGCGTKVEIKGGGGSGGGGSGGGGSGGGGSQVQLVQSGGGLVQPG | |
| A5) | GSQRLSCAASGFTFTSYAMSWVRQAPGKCLEWVSAISGTGDSTFYAD | |
| | SVKGRFTFSRDNSKNTLYLQMNSLRAEDTAVYYCAKDLGWLQYGLF | |
| | DYWGQGTLVTVSS (SEQ ID NO: 573) | |
| | GB10 (VH-VL): | |
| | QVQLVQSGGGLVQPGGSQRLSCAASGFTFTSYAMSWVRQAPGKCLE | |
| | WVSAISGTGDSTFYADSVKGRFTFSRDNSKNTLYLQMNSLRAEDTAV | |
| | YYCAKDLGWLQYGLFDYWGQGTLVTVSSGGGGSGGGGSGGGGSGG | |
| | GGSDIQLTQSPATLSVSPGERATLSCRASQSVSSSYLAWYQQKPGQAP | |
| | RLLIYGASSRATGIPDRFSGSGSGTDFTLTISRLEPEDFAVYYCQQYNN | |
| | WPLTFGCGTKVEIK (SEQ ID NO: 598) | |
| Cognate Pair | QVQLQESGPGLVRPSGTLSLTC | DIVMTQTPATLSASVGDRVTITCR |
| A6 (derived | AVSGGSISSPTWWSWVRQPPGK | ASQSVRSNLAWYQQKPGQAPRLLI |
| from | CLEWIGEIHPSGRTNYNPSLKSR | YGASTRATGIPARFSGSGSGTEFTL |
| 1079_H05- | VTISVDKSKNQFSLKLGSVTAA | TISSLQSEDFAVYYCQQYNNWPTF |
| CEACAM5) | DTAVYYCAREGFYYGSGNYYY | GCGTRLEIK (SEQ ID NO: 600) |
| | FDYWGQGTLVTVSS (SEQ ID | CDR1: RASQSVRSNLA (SEQ ID |
| | NO: 599) | NO: 62) |
| | CDR1: GGSISSPT (SEQ ID | CDR2: GASTRAT (SEQ ID NO: 63) |
| | NO: 56)(Chothia) or SPTWWS | CDR3: QQYNNWPT (SEQ ID NO: 64) |
| | (SEQ ID NO: 57)(Kabat) | |
| | CDR2: HPSGR (SEQ ID NO: 58) | |
| | (Chothia) or | |
| | EIHPSGRTNYNPSLKS (SEQ ID | |
| | NO: 59)(Kabat) | |
| | CDR3: EGFYYGSGNYYYFDY | |
| | (SEQ ID NO: 60) | |
| scFv for | GB11(VH-VL): | |
| GB11/ GB12 | QVQLQESGPGLVRPSGTLSLTCAVSGGSISSPTWWSWVRQPPGKCLE | |
| (derived from | WIGEIHPSGRTNYNPSLKSRVTISVDKSKNQFSLKLGSVTAADTAVYY | |
| Cognate Pair | CAREGFYYGSGNYYYFDYWGQGTLVTVSSGGGGSGGGGSGGGGSGG | |
| A6) | GGSDIVMTQTPATLSASVGDRVTITCRASQSVRSNLAWYQQKPGQAP | |
| | RLLIYGASTRATGIPARFSGSGSGTEFTLTISSLQSEDFAVYYCQQYNN | |
| | WPTFGCGTRLEIK (SEQ ID NO: 574) | |
| | GB12 (VL-VH): | |
| | DIVMTQTPATLSASVGDRVTITCRASQSVRSNLAWYQQKPGQAPRLLI | |
| | YGASTRATGIPARFSGSGSGTEFTLTISSLQSEDFAVYYCQQYNNWPTF | |
| | GCGTRLEIKGGGGSGGGGSGGGGSGGGGSQVQLQESGPGLVRPSGTL | |
| | SLTCAVSGGSISSPTWWSWVRQPPGKCLEWIGEIHPSGRTNYNPSLKS | |
| | RVTISVDKSKNQFSLKLGSVTAADTAVYYCAREGFYYGSGNYYYFDY | |
| | WGQGTLVTVSS (SEQ ID NO: 601) | |
| Cognate Pair | EVQLQESGPGLVRPSGTLSLTC | DIVMTQTPATLSASVGDRVTITCR |
| A7 | AVSGGSISSPTWWSWVRQPPGK | ASQSVRSNLAWYQQKPGQAPRLLI |
| (VH derived | CLEWIGEIHPSGRTNYNPSLKSR | YGASTRATGIPARFSGSGSGTEFTL |
| from | VTISVDKSKNQFSLKLGSVTAA | TISSLQSEDFAVYYCQQYNNWPTF |
| 1079_H05- | DTAVYYCAREGFYYGSGNYYY | GCGTRLEIK (SEQ ID NO: 603) |
| CEACAM5_ | FDYWGQGTLVTVSS (SEQ ID | CDR1: RASQSVRSNLA (SEQ ID |
| VH_Variant 1 | NO: 602) | NO: 62) |
| (QE)) | CDR1: GGSISSPT (SEQ ID | CDR2: GASTRAT (SEQ ID NO: 63) |
| | NO: 56)(Chothia) or SPTWWS | CDR3: QQYNNWPT (SEQ ID NO: 64) |
| | (SEQ ID NO: 57)(Kabat) | |
| | CDR2: HPSGR (SEQ ID NO: 58) | |
| | (Chothia) or | |
| | EIHPSGRTNYNPSLKS (SEQ ID | |
| | NO: 59)(Kabat) | |
| | CDR3: EGFYYGSGNYYYFDY | |
| | (SEQ ID NO: 60) | |
| scFv for | GB13 (VH-VL): | |
| GB13/ GB14 | EVQLQESGPGLVRPSGTLSLTCAVSGGSISSPTWWSWVRQPPGKCLE | |
| (derived from | WIGEIHPSGRTNYNPSLKSRVTISVDKSKNQFSLKLGSVTAADTAVYY | |
| Cognate Pair | CAREGFYYGSGNYYYFDYWGQGTLVTVSSGGGGSGGGGSGGGGSGG | |
| A7) | GGSDIVMTQTPATLSASVGDRVTITCRASQSVRSNLAWYQQKPGQAP | |
| | RLLIYGASTRATGIPARFSGSGSGTEFTLTISSLQSEDFAVYYCQQYNN | |
| | WPTFGCGTRLEIK (SEQ ID NO: 575) | |
| | GB14 (VL-VH): | |
| | DIVMTQTPATLSASVGDRVTITCRASQSVRSNLAWYQQKPGQAPRLLI | |
| | YGASTRATGIPARFSGSGSGTEFTLTISSLQSEDFAVYYCQQYNNWPTF | |
| | GCGTRLEIKGGGGSGGGGSGGGGSGGGGSDIVMTQTPATLSASVGDR | |
| | VTITCRASQSVRSNLAWYQQKPGQAPRLLIYGASTRATGIPARFSGSG | |
| | SGTEFTLTISSLQSEDFAVYYCQQYNNWPTFGCGTRLEIK (SEQ ID | |
| | NO: 604) | |
| Cognate Pair | QVQLQESGPGLVRPSGTLSLTC | DIVMTQTPATLSASVGDRVTITCR |
| A8 | AVSGGSISSPTWWSWVRQPPGK | ASQSVRSNLAWYQQKPGQAPRLLI |
| (derived from | CLEWIGEIHPSGRTNYNPSLKSR | YGASTRATGIPARFSGSGSGTEFTL |
| 1079_H05- | VTISVDKSKNQFSLKLGSVTAA | TISSLQSEDFAVYYCQQYNNWPTF |
| CEACAM5) | DTAVYYCAREGFYYGSGNYYY | GCGTRLEIK (SEQ ID NO: 606) |
| | FDYWGQGTLVTVSS (SEQ ID | CDR1: RASQSVRSNLA (SEQ ID |
| | NO: 605) | NO: 62) |
| | CDR1: GGSISSPT (SEQ ID | CDR2: GASTRAT (SEQ ID NO: 63) |
| | NO: 56)(Chothia) or SPTWWS | CDR3: QQYNNWPT (SEQ ID NO: 64) |
| | (SEQ ID NO: 57)(Kabat) | |
| | CDR2: HPSGR (SEQ ID NO: 58) | |
| | (Chothia) or | |
| | EIHPSGRTNYNPSLKS (SEQ ID | |
| | NO: 59)(Kabat) | |
| | CDR3: EGFYYGSGNYYYFDY | |
| | (SEQ ID NO: 60) | |
| scFv for | GB15 (VL-VH): | |
| GB15/ GB16 | DIVMTQTPATLSASVGDRVTITCRASQSVRSNLAWYQQKPGQAPRLLI | |
| (derived from | YGASTRATGIPARFSGSGSGTEFTLTISSLQSEDFAVYYCQQYNNWPTF | |
| Cognate Pair | GCGTRLEIKGGGGSGGGGSGGGGSGGGGSQVQLQESGPGLVRPSGTL | |
| A8) | SLTCAVSGGSISSPTWWSWVRQPPGKCLEWIGEIHPSGRTNYNPSLKS | |
| | RVTISVDKSKNQFSLKLGSVTAADTAVYYCAREGFYYGSGNYYYFDY | |
| | WGQGTLVTVSS (SEQ ID NO: 576) | |
| | GB16 (VH-VL): | |
| | QVQLQESGPGLVRPSGTLSLTCAVSGGSISSPTWWSWVRQPPGKCLE | |
| | WIGEIHPSGRTNYNPSLKSRVTISVDKSKNQFSLKLGSVTAADTAVYY | |
| | CAREGFYYGSGNYYYFDYWGQGTLVTVSSGGGGSGGGGSGGGGSGG | |
| | GGSDIVMTQTPATLSASVGDRVTITCRASQSVRSNLAWYQQKPGQAP | |
| | RLLIYGASTRATGIPARFSGSGSGTEFTLTISSLQSEDFAVYYCQQYNN | |
| | WPTFGCGTRLEIK (SEQ ID NO: 607) | |
| Cognate Pair | QVQLVESGGDVVQPGRSLRLSC | EIVMTQSPLSLPVTPGEPASISCRSS |
| A9 | AASGFILSNYGMHWVRQAPGK | QSLLHSNGYNYLDWYLQKPGQSP |
| (derived from | CLEWVAAMWYDGSNNYYEDS | QLLISLGSIRASGVPDRFSGSGSGT |
| 7A10.A7- | VKGRFTISRDNSKNTLYLQMNS | NFTLTISRVEAEDVGFYYCMQALQ |
| CEACAM5- | LRAEDTAVYYCARERVSRHFD | TPRTFGCGTKVDIT (SEQ ID |
| B.01) | WHYYYGMDVWGQGTTVTVSS | NO: 609) |
| | (SEQ ID NO: 608) | CDR1: RSSQSLLHSNGYNYLD |
| | CDR1: GFILSNY (SEQ ID NO: 68) | (SEQ ID NO: 74) |
| | (Chothia) or NYGMH (SEQ ID | CDR2: LGSIRAS (SEQ ID NO: 75) |
| | NO: 69)(Kabat) | CDR3: MQALQTPRT (SEQ ID |
| | CDR2: WYDGSN (SEQ ID NO: 70) | NO: 76) |
| | (Chothia) or | |
| | AMWYDGSNNYYEDSVKG | |
| | (SEQ ID NO: 71)(Kabat) | |
| | CDR3: | |
| | ERVSRHFDWHYYYGMDV (SEQ | |
| | ID NO: 72) | |
| scFv for | GB17 (VH-VL): | |
| GB17/GB18 | QVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A9) | GGGGSGGGGSEIVMTQSPLSLPVTPGEPASISCRSSQSLLHSNGYNYLD | |
| | WYLQKPGQSPQLLISLGSIRASGVPDRFSGSGSGTNFTLTISRVEAEDV | |
| | GFYYCMQALQTPRTFGCGTKVDIT (SEQ ID NO: 577) | |
| | GB18 (VL-VH): | |
| | EIVMTQSPLSLPVTPGEPASISCRSSQSLLHSNGYNYLDWYLQKPGQSP | |
| | QLLISLGSIRASGVPDRFSGSGSGTNFTLTISRVEAEDVGFYYCMQALQ | |
| | TPRTFGCGTKVDITGGGGSGGGGSGGGGSGGGGSEIVMTQSPLSLPVT | |
| | PGEPASISCRSSQSLLHSNGYNYLDWYLQKPGQSPQLLISLGSIRASGV | |
| | PDRFSGSGSGTNFTLTISRVEAEDVGFYYCMQALQTPRTFGCGTKVDI | |
| | T (SEQ ID NO: 610) | |
| Cognate Pair | QVQLVESGGDVVQPGRSLRLSC | EIVMTQSPLSLPVTPGEPASISCRSS |
| A10 | AASGFILSNYGMHWVRQAPGK | QSLLHSNGYNYLDWYLQKPGQSP |
| (VH derived | CLEWVAAMWYDGSNNYYEDS | QLLISLGSIRASGVPDRFSGSGSGT |
| from | VKGRFTISRDNSKNTLYLQMNS | DFTLTISRVEAEDVGFYYCMQALQ |
| 7A10.A7- | LRAEDTAVYYCARERVSRHFD | TPRTFGCGTKVDIT (SEQ ID |
| CEACAM5- | WHYYYGMDVWGQGTTVTVSS | NO: 612) |
| B.01 and VL | (SEQ ID NO: 611) | CDR1: RSSQSLLHSNGYNYLD |
| derived from | CDR1: GFILSNY (SEQ ID NO: 68) | (SEQ ID NO: 74) |
| 7A10.A7- | (Chothia) or NYGMH (SEQ ID | CDR2: LGSIRAS (SEQ ID NO: 75) |
| CEACAM5- | NO: 69)(Kabat) | CDR3: MQALQTPRT (SEQ ID |
| B.01- | CDR2: WYDGSN (SEQ ID NO: 70) | NO: 76) |
| N(L70)D VL) | (Chothia) or | |
| | AMWYDGSNNYYEDSVKG | |
| | (SEQ ID NO: 71)(Kabat) | |
| | CDR3: | |
| | ERVSRHFDWHYYYGMDV (SEQ | |
| | ID NO: 72) | |
| scFv for | GB19 (VH-VL): | |
| GB19/GB20 | QVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A10) | GGGGSGGGGSEIVMTQSPLSLPVTPGEPASISCRSSQSLLHSNGYNYLD | |
| | WYLQKPGQSPQLLISLGSIRASGVPDRFSGSGSGTDFTLTISRVEAEDV | |
| | GFYYCMQALQTPRTFGCGTKVDIT (SEQ ID NO: 578) | |
| | GB20 (VL-VH): | |
| | EIVMTQSPLSLPVTPGEPASISCRSSQSLLHSNGYNYLDWYLQKPGQSP | |
| | QLLISLGSIRASGVPDRFSGSGSGTDFTLTISRVEAEDVGFYYCMQALQ | |
| | TPRTFGCGTKVDITGGGGSGGGGSGGGGSGGGGSQVQLVESGGDVVQ | |
| | PGRSLRLSCAASGFILSNYGMHWVRQAPGKCLEWVAAMWYDGSNN | |
| | YYEDSVKGRFTISRDNSKNTLYLQMNSLRAEDTAVYYCARERVSRHF | |
| | DWHYYYGMDVWGQGTTVTVSS (SEQ ID NO: 613) | |
| Cognate Pair | QVQLVESGGDVVQPGRSLRLSC | EIVMTQSPLSLPVTPGEPASISCRSS |
| A11 | AASGFILSNYGMHWVRQAPGK | QSLLHSNGYNYLDWYLQKPGQSP |
| (VH derived | CLEWVAAMWYDGSNNYYEDS | QLLISLGSIRASGVPDRFSGSGSGT |
| from | VKGRFTISRDNSKNTLYLQMNS | QFTLTISRVEAEDVGFYYCMQALQ |
| 7A10.A7- | LRAEDTAVYYCARERVSRHFD | TPRTFGCGTKVDIT (SEQ ID |
| CEACAM5- | WHYYYGMDVWGQGTTVTVSS | NO: 615) |
| B.01 and VL | (SEQ ID NO: 614) | CDR1: RSSQSLLHSNGYNYLD |
| derived from | CDR1: GFILSNY (SEQ ID NO: 68) | (SEQ ID NO: 74) |
| 7A10.A7- | (Chothia) or NYGMH (SEQ ID | CDR2: LGSIRAS (SEQ ID NO: 75) |
| CEACAM5- | NO: 69)(Kabat) | CDR3: MQALQTPRT (SEQ ID |
| B.01- | CDR2: WYDGSN (SEQ ID NO: 70) | NO: 76) |
| N(L70)Q VL) | (Chothia) or | |
| | AMWYDGSNNYYEDSVKG | |
| | (SEQ ID NO: 71)(Kabat) | |
| | CDR3: | |
| | ERVSRHFDWHYYYGMDV (SEQ | |
| | ID NO: 72) | |
| scFv for | GB21 (VH-VL): | |
| GB21/ GB22 | QVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A11) | GGGGSGGGGSEIVMTQSPLSLPVTPGEPASISCRSSQSLLHSNGYNYLD | |
| | WYLQKPGQSPQLLISLGSIRASGVPDRFSGSGSGTQFTLTISRVEAEDV | |
| | GFYYCMQALQTPRTFGCGTKVDIT (SEQ ID NO: 579) | |
| | GB22 (VL-VH): | |
| | EIVMTQSPLSLPVTPGEPASISCRSSQSLLHSNGYNYLDWYLQKPGQSP | |
| | QLLISLGSIRASGVPDRFSGSGSGTQFTLTISRVEAEDVGFYYCMQALQ | |
| | TPRTFGCGTKVDITGGGGSGGGGSGGGGSGGGGSQVQLVESGGDVVQ | |
| | PGRSLRLSCAASGFILSNYGMHWVRQAPGKCLEWVAAMWYDGSNN | |
| | YYEDSVKGRFTISRDNSKNTLYLQMNSLRAEDTAVYYCARERVSRHF | |
| | DWHYYYGMDVWGQGTTVTVSS (SEQ ID NO: 616) | |
| Cognate Pair | QVQLVESGGDVVQPGRSLRLSC | EIVMTQSPLSLPVTPGEPASISCRSS |
| A12 | AASGFILSNYGMHWVRQAPGK | QSLLHSNGYNYLDWYLQKPGQSP |
| (VH derived | CLEWVAAMWYDGSNNYYEDS | QLLISLGSIRASGVPDRFSGSGSGT |
| from | VKGRFTISRDNSKNTLYLQMNS | NFALTISRVEAEDVGFYYCMQAL |
| 7A10.A7- | LRAEDTAVYYCARERVSRHFD | QTPRTFGCGTKVDIT (SEQ ID |
| CEACAM5- | WHYYYGMDVWGQGTTVTVSS | NO: 618) |
| B.01 and VL | (SEQ ID NO: 617) | CDR1: RSSQSLLHSNGYNYLD |
| derived from | CDR1: GFILSNY (SEQ ID NO: 68) | (SEQ ID NO: 74) |
| 7A10.A7- | (Chothia) or NYGMH (SEQ ID | CDR2: LGSIRAS (SEQ ID NO: 75) |
| CEACAM5- | NO: 69)(Kabat) | CDR3: MQALQTPRT (SEQ ID |
| B.01_VL_ | CDR2: WYDGSN (SEQ ID NO: 70) | NO: 76) |
| Variant 1_(TA)) | (Chothia) or | |
| | AMWYDGSNNYYEDSVKG | |
| | (SEQ ID NO: 71)(Kabat) | |
| | CDR3: | |
| | ERVSRHFDWHYYYGMDV (SEQ | |
| | ID NO: 72) | |
| scFv for | GB23 (VH-VL): | |
| GB23/GB24 | QVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A12) | GGGGSGGGGSEIVMTQSPLSLPVTPGEPASISCRSSQSLLHSNGYNYLD | |
| AB0246 (TA) | WYLQKPGQSPQLLISLGSIRASGVPDRFSGSGSGTNFALTISRVEAEDV | |
| | GFYYCMQALQTPRTFGCGTKVDIT (SEQ ID NO: 580) | |
| | GB24 (VL-VH): | |
| | EIVMTQSPLSLPVTPGEPASISCRSSQSLLHSNGYNYLDWYLQKPGQSP | |
| | QLLISLGSIRASGVPDRFSGSGSGTNFALTISRVEAEDVGFYYCMQALQ | |
| | TPRTFGCGTKVDITGGGGSGGGGSGGGGSGGGGSEIVMTQSPLSLPVT | |
| | PGEPASISCRSSQSLLHSNGYNYLDWYLQKPGQSPQLLISLGSIRASGV | |
| | PDRFSGSGSGTNFALTISRVEAEDVGFYYCMQALQTPRTFGCGTKVDI | |
| | T (SEQ ID NO: 619) | |
| Cognate Pair | EVQLVESGGDVVQPGRSLRLSC | EIVMTQSPLSLPVTPGEPASISCRSS |
| A13 (VH is | AASGFILSNYGMHWVRQAPGK | QSLLHSNGYNYLDWYLQKPGQSP |
| derived from | CLEWVAAMWYDGSNNYYEDS | QLLISLGSIRASGVPDRFSGSGSGT |
| 7A10.A7- | VKGRFTISRDNSKNTLYLQMNS | DFTLTISRVEAEDVGFYYCMQALQ |
| CEACAM5- | LRAEDTAVYYCARERVSRHFD | TPRTFGCGTKVDIT (SEQ ID |
| B.01_VH_ | WHYYYGMDVWGQGTTVTVSS | NO: 621) |
| Variant 1_(QE) | (SEQ ID NO: 620) | CDR1: RSSQSLLHSNGYNYLD |
| and VL is | CDR1: GFILSNY (SEQ ID NO: 68) | (SEQ ID NO: 74) |
| derived from | (Chothia) or NYGMH (SEQ ID | CDR2: LGSIRAS (SEQ ID NO: 75) |
| 7A10.A7- | NO: 69)(Kabat) | CDR3: MQALQTPRT (SEQ ID |
| CEACAM5- | CDR2: WYDGSN (SEQ ID NO: 70) | NO: 76) |
| B.01- | (Chothia) or | |
| N(L70)D VL) | AMWYDGSNNYYEDSVKG | |
| | (SEQ ID NO: 71)(Kabat) | |
| | CDR3: | |
| | ERVSRHFDWHYYYGMDV (SEQ | |
| | ID NO: 72) | |
| scFv for | GC25 (VH-VL) | |
| GB25/GB26 | EVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A13) | GGGGSGGGGSEIVMTQSPLSLPVTPGEPASISCRSSQSLLHSNGYNYLD | |
| | WYLQKPGQSPQLLISLGSIRASGVPDRFSGSGSGTDFTLTISRVEAEDV | |
| | GFYYCMQALQTPRTFGCGTKVDIT (SEQ ID NO: 581) | |
| | GB26 (VL-VH): | |
| | EIVMTQSPLSLPVTPGEPASISCRSSQSLLHSNGYNYLDWYLQKPGQSP | |
| | QLLISLGSIRASGVPDRFSGSGSGTDFTLTISRVEAEDVGFYYCMQALQ | |
| | TPRTFGCGTKVDITGGGGSGGGGSGGGGSGGGGSEVQLVESGGDVVQ | |
| | PGRSLRLSCAASGFILSNYGMHWVRQAPGKCLEWVAAMWYDGSNN | |
| | YYEDSVKGRFTISRDNSKNTLYLQMNSLRAEDTAVYYCARERVSRHF | |
| | DWHYYYGMDVWGQGTTVTVSS (SEQ ID NO: 622) | |
| Cognate Pair | EVQLVESGGDVVQPGRSLRLSC | EIVMTQSPLSLPVTPGEPASISCRSS |
| A14 (VH is | AASGFILSNYGMHWVRQAPGK | QSLLHSSGYNYLDWYLQKPGQSP |
| derived from | CLEWVAAMWYDGSNNYYEDS | QLLISLGSIRASGVPDRFSGSGSGT |
| 7A10.A7- | VKGRFTISRDNSKNTLYLQMNS | DFTLTISRVEAEDVGFYYCMQALQ |
| CEACAM5- | LRAEDTAVYYCARERVSRHFD | TPRTFGCGTKVDIT (SEQ ID |
| B.01_VH_ | WHYYYGMDVWGQGTTVTVSS | NO: 624) |
| Variant 1_(QE) | (SEQ ID NO: 623) | CDR1: RSSQSLLHSSGYNYLD (SEQ |
| and VL is | CDR1: GFILSNY (SEQ ID NO: 68) | ID NO: 630) |
| derived from | (Chothia) or NYGMH (SEQ ID | CDR2: LGSIRAS (SEQ ID NO: 75) |
| 7A10.A7- | NO: 69)(Kabat) | CDR3: MQALQTPRT (SEQ ID |
| CEACAM5- | CDR2: WYDGSN (SEQ ID NO: 70) | NO: 76) |
| B.01- | (Chothia) or | |
| N(L70)D, | AMWYDGSNNYYEDSVKG | |
| N(L28)S VL) | (SEQ ID NO: 71)(Kabat) | |
| | CDR3: | |
| | ERVSRHFDWHYYYGMDV (SEQ | |
| | ID NO: 72) | |
| scFv for | GB27 (VH-VL): | |
| GB27/GB28 | EVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A14) | GGGGSGGGGSEIVMTQSPLSLPVTPGEPASISCRSSQSLLHSSGYNYLD | |
| | WYLQKPGQSPQLLISLGSIRASGVPDRFSGSGSGTDFTLTISRVEAEDV | |
| | GFYYCMQALQTPRTFGCGTKVDIT (SEQ ID NO: 582) | |
| | GB28 (VL-VH): | |
| | EIVMTQSPLSLPVTPGEPASISCRSSQSLLHSSGYNYLDWYLQKPGQSP | |
| | QLLISLGSIRASGVPDRFSGSGSGTDFTLTISRVEAEDVGFYYCMQALQ | |
| | TPRTFGCGTKVDITGGGGSGGGGSGGGGSGGGGSEVQLVESGGDVVQ | |
| | PGRSLRLSCAASGFILSNYGMHWVRQAPGKCLEWVAAMWYDGSNN | |
| | YYEDSVKGRFTISRDNSKNTLYLQMNSLRAEDTAVYYCARERVSRHF | |
| | DWHYYYGMDVWGQGTTVTVSS (SEQ ID NO: 626) | |
| Cognate Pair | EVQLVESGGDVVQPGRSLRLSC | EIVMTQSPLSLPVTPGEPASISCRSS |
| A15 (VH is | AASGFILSNYGMHWVRQAPGK | QSLLHSQGYNYLDWYLQKPGQSP |
| derived from | CLEWVAAMWYDGSNNYYEDS | QLLISLGSIRASGVPDRFSGSGSGT |
| 7A10.A7- | VKGRFTISRDNSKNTLYLQMNS | DFTLTISRVEAEDVGFYYCMQALQ |
| CEACAM5- | LRAEDTAVYYCARERVSRHFD | TPRTFGCGTKVDIT (SEQ ID |
| B.01_VH | WHYYYGMDVWGQGTTVTVSS | NO: 628) |
| Variant 1_(QE) | (SEQ ID NO: 627) | CDR1: RSSQSLLHSQGYNYLD |
| and VL is | CDR1: GFILSNY (SEQ ID NO: 68) | (SEQ ID NO: 629) |
| derived from | (Chothia) or NYGMH (SEQ ID | CDR2: LGSIRAS (SEQ ID NO: 75) |
| 7A10.A7- | NO: 69)(Kabat) | CDR3: MQALQTPRT (SEQ ID |
| CEACAM5- | CDR2: WYDGSN (SEQ ID NO: 70) | NO: 76) |
| B.01- | (Chothia) or | |
| N(L70)D, | AMWYDGSNNYYEDSVKG | |
| N(L28)Q VL) | (SEQ ID NO: 71)(Kabat) | |
| | CDR3: | |
| | ERVSRHFDWHYYYGMDV (SEQ | |
| | ID NO: 72) | |
| scFv for | GB29 (VH-VL): | |
| GB29/GB30 | EVQLVESGGDVVQPGRSLRLSCAASGFILSNYGMHWVRQAPGKCLE | |
| (derived from | WVAAMWYDGSNNYYEDSVKGRFTISRDNSKNTLYLQMNSLRAEDT | |
| Cognate Pair | AVYYCARERVSRHFDWHYYYGMDVWGQGTTVTVSSGGGGSGGGGS | |
| A15) | GGGGSGGGGSEIVMTQSPLSLPVTPGEPASISCRSSQSLLHSQGYNYLD | |
| | WYLQKPGQSPQLLISLGSIRASGVPDRFSGSGSGTDFTLTISRVEAEDV | |
| | GFYYCMQALQTPRTFGCGTKVDIT (SEQ ID NO: 583) | |
| | GB30 (VL-VH): | |
| | EIVMTQSPLSLPVTPGEPASISCRSSQSLLHSQGYNYLDWYLQKPGQSP | |
| | QLLISLGSIRASGVPDRFSGSGSGTDFTLTISRVEAEDVGFYYCMQALQ | |
| | TPRTFGCGTKVDITGGGGSGGGGSGGGGSGGGGSEVQLVESGGDVVQ | |
| | PGRSLRLSCAASGFILSNYGMHWVRQAPGKCLEWVAAMWYDGSNN | |
| | YYEDSVKGRFTISRDNSKNTLYLQMNSLRAEDTAVYYCARERVSRHF | |
| | DWHYYYGMDVWGQGTTVTVSS (SEQ ID NO: 490) | |
| Cognate Pair | EVQLVESGGDVVQPGRSLRLSC | EIVMTQSPLSLPVTPGEPASISCRSS |
| A16 (VH is | AASGFILSNYGMHWVRQAPGK | QSLLHSNAYNYLDWYLQKPGQSP |
| derived from | CLEWVAAMWYDGSNNYYEDS | QLLISLGSIRASGVPDRFSGSGSGT |
| 7A10.A7- | VKGRFTISRDNSKNTLYLQMNS | DFTLTISRVEAEDVGFYYCMQALQ |
| CEACAM5- | LRAEDTAVYYCARERVSRHFD | TPRTFGCGTKVDIT(SEQ ID |
| B.01_VH_ | WHYYYGMDVWGQGTTVTVSS | NO: 485) |
| Variant 1_(QE) | (SEQ ID NO: 471) | CDR1: RSSQSLLHSNAYNYLD |
| and VL is | CDR1: GFILSNY (SEQ ID NO: 68) | (SEQ ID NO: 625) |
| derived from | (Chothia) or NYGMH (SEQ ID | CDR2: LGSIRAS (SEQ ID NO: 75) |
| 7A10.A7- | NO: 69)(Kabat) | CDR3: MQALQTPRT (SEQ ID |
| CEACAM5- | CDR2: WYDGSN (SEQ ID NO: 70) | NO: 76) |
| B.01- | (Chothia) or | |
| N(L70)D, | AMWYDGSNNYYEDSVKG | |
| G(L29)A VL) | (SEQ ID NO: 71)(Kabat) | |
| | CDR3: | |
| | ERVSRHFDWHYYYGMDV (SEQ | |
| | ID NO: 72) | |

| | |
|---|---|
| scFv for | GB31 (VH-VL): |
| GB31/ GB32 | EVQLVESGGDVVQPGRSLRLSCAASGFILSNYGMHWVRQAPGKCLE |
| (derived from | WVAAMWYDGSNNYYEDSVKGRFTISRDNSKNTLYLQMNSLRAEDT |
| Cognate Pair | AVYYCARERVSRHFDWHYYYGMDVWGQGTTVTVSSGGGGSGGGGS |
| A16) | GGGGSGGGGSEIVMTQSPLSLPVTPGEPASISCRSSQSLLHSNAYNYLD |
| | WYLQKPGQSPQLLISLGSIRASGVPDRFSGSGSGTDFTLTISRVEAEDV |
| | GFYYCMQALQTPRTFGCGTKVDIT (SEQ ID NO: 584) |
| | GB32 (VL-VH): |
| | EIVMTQSPLSLPVTPGEPASISCRSSQSLLHSNAYNYLDWYLQKPGQSP |
| | QLLISLGSIRASGVPDRFSGSGSGTDFTLTISRVEAEDVGFYYCMQALQ |
| | TPRTFGCGTKVDITGGGGSGGGGSGGGGSGGGGSEVQLVESGGDVVQ |
| | PGRSLRLSCAASGFILSNYGMHWVRQAPGKCLEWVAAMWYDGSNN |
| | YYEDSVKGRFTISRDNSKNTLYLQMNSLRAEDTAVYYCARERVSRHF |
| | DWHYYYGMDVWGQGTTVTVSS (SEQ ID NO: 585) |

Alternatively, novel antigen-binding sites that can bind to CEACAM5 can be identified by screening for binding to the amino acid sequence defined by SEQ ID NO:391, a mature extracellular fragment thereof, or a fragment containing a domain of CEACAM5 (see, e.g., U.S. Pat. Nos. 9,771,431, 9,617,345, and 8,470,994, and U.S. application Ser. Nos. 15/683,087 and 14/515,765).

An exemplary sequence for a human CEACAM5 isoform is provided below and can be obtained from GenBank database under accession number NP_004354.

(SEQ ID NO: 391)
MESPSAPPHRWCIPWQRLLLTASLLTFWNPPTTAKLTIESTPFNVAEGKE
VLLLVHNLPQHLFGYSWYKGERVDGNRQIIGYVIGTQQATPGPAYSGREI
IYPNASLLIQNIIQNDTGFYTLHVIKSDLVNEEATGQFRVYPELPKPSIS
SNNSKPVEDKDAVAFTCEPETQDATYLWWVNNQSLPVSPRLQLSNGNRTL
TLFNVTRNDTASYKCETQNPVSARRSDSVILNVLYGPDAPTISPLNTSYR
SGENLNLSCHAASNPPAQYSWFVNGTFQQSTQELFIPNITVNNSGSYTCQ
AHNSDTGLNRTTVTTITVYAEPPKPFITSNNSNPVEDEDAVALTCEPEIQ
NTTYLWWVNNQSLPVSPRLQLSNDNRTLTLLSVTRNDVGPYECGIQNELS
VDHSDPVILNVLYGPDDPTISPSYTYYRPGVNLSLSCHAASNPPAQYSWL
IDGNIQQHTQELFISNITEKNSGLYTCQANNSASGHSRTTVKTITVSAEL
PKPSISSNNSKPVEDKDAVAFTCEPEAQNTTYLWWVNGQSLPVSPRLQLS
NGNRTLTLFNVTRNDARAYVCGIQNSVSANRSDPVTLDVLYGPDTPIISP
PDSSYLSGANLNLSCHSASNPSPQYSWRINGIPQQHTQVLFIAKITPNNN
GTYACFVSNLATGRNNSIVKSITVSASGTSPGLSAGATVGIMIGVLVGVA
LI

In certain embodiments, the second antigen-binding site that binds CEACAM5 (e.g., human CEACAM5, e.g., cynomolgus monkey CEACAM5) comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antigen-binding site disclosed in Table 2, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antigen-binding site disclosed in Table 2. In certain embodiments, the second antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antigen-binding site disclosed in Table 2. In certain embodiments, the second antigen-binding site comprises the heavy chain CDR1, CDR2, and CDR3 sequences and the light chain CDR1, CDR2, and CDR3 sequences of an antigen-binding site disclosed in Table 2.

In certain embodiments, the second antigen-binding site is related to an scFv in Table 2. For example, in certain embodiments, the second antigen-binding site comprises a VH sequence that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VH in Table 2. In certain embodiments, the second antigen-binding site comprises a VL sequence that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VL in Table 2. In certain embodiments, the second antigen-binding site comprises a VH sequence that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VH in Table 2, and a VL sequence that comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence of a VL in Table 2, wherein the VH and VL in Table 2 are selected from a cognate pair of sequences.

In certain embodiments, the VH comprises CDR1, CDR2, and CDR3 selected from a cognate pair of sequences listed in Table 2. In certain embodiments, the VL comprises CDR1, CDR2, and CDR3 selected from a cognate pair of sequences listed in Table 2. In certain embodiments, the second antigen-binding site comprises (a) a VH that comprises a CDR1, CDR2, and CDR3 of a VH sequence listed in Table 2; and (b) a VL that comprises CDR1, CDR2, and CDR3 of a VL sequence listed in Table 2, wherein the VH and VL sequences are selected from a cognate pair of sequences listed in Table 2.

As used herein, the term “cognate pair” refers to a VH and a VL that form an antigen-binding site. In some embodiments, “cognate pair” refers to a VH and VL pairing as shown in Table 2. In some embodiments, “cognate pair” refers to a VH and VL pairing as shown in Table 3.

As used in Table 2, the term “derived” when applied to a VH, VL or CDR, refers to an amino acid sequence that has additional mutations (e.g., substitutions, deletions, etc.) relative to the referenced sequence. For example, the VH of Cognate Pair A1 (SEQ ID NO:567) in Table 2 was derived from the VH of PH_420-CEACAM5 (shown in Table 3). Relative to the VH of PH_420-CEACAM5, SEQ ID NO:567 has a cysteine mutation. The VL of Cognate Pair A1 (SEQ ID NO:568) in Table 2 was derived from the VL of PH_420-CEACAM5 (shown in Table 3). Relative to the VL of PH_420-CEACAM5, SEQ ID NO:568 has a cysteine mutation.

As used in Table 2, the term “derived” when applied to an scFv refers to an amino acid sequence that has additional mutations and/or a linker sequence. For example, scFv for GB1 is derived from Cognate Pair A1 in Table 2 and comprises the VH and VL sequences of Cognate Pair A1 in Table 2, as well as a linker sequence (e.g., the (G₄S)₄ linker sequence (SEQ ID NO: 532)).

Table 3 lists exemplary sequences of heavy chain variable domains and light chain variable domains that, in combination, e.g., as a cognate pair, can bind to CEACAM5. CDR sequences are identified under Chothia and Kabat numbering as indicated.

TABLE 3
Sequences of Exemplary Antigen-Binding Sites that Bind CEACAM5

| | | | |
|---|---|---|---|
| Cognate | Clones and | | |
| Pair | Variants | VH | VL |

Tier 1

| | | | |
|---|---|---|---|
| 1. | Clone | QVQLVQSGGGLVQPGGSLRL | DIRMTQSPSTLSASVGDRVTI |
| | PH_420- | SCAASGFTFSSYAMSWVRDI | TCWASQSISSWLAWYQQKP |
| | CEACAM5 | QMTQSPSSQAPGKGLEWVSA | GKAPKLLIYKASSLESGVPS |
| | | IFNSGGSTYYADSVKGRFTVS | RFSGSGSGTEFTLTISSLQPD |
| | | RDNSKNTLYLQMNSLRAEDT | DFATYYCQQYNSYSYTFGQ |
| | | ALYYCAKDLGGYNYGLFDY | GTKLEIK (SEQ ID NO: 10) |
| | | WGQGTLVTVSS (SEQ ID | CDR1: WASQSISSWLA (SEQ |
| | | NO: 1) | ID NO: 7) |
| | | CDR1: GFTFSSY (SEQ ID | CDR2: KASSLES (SEQ ID |
| | | NO: 2)(Chothia) or SYAMS | NO: 8) |
| | | (SEQ ID NO: 3)(Kabat) | CDR3: QQYNSYSYT (SEQ ID |
| | | CDR2: FNSGGS (SEQ ID NO: 4) | NO: 9) |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_Variant | PGKGLEWVSAIFNSGGSTYY | |
| | 1 (QE) | ADSVKGRFTVSRDNSKNTLY | |
| | | LQMNSLRAEDTALYYCAKDL | |
| | | GGYNYGLFDYWGQGTLVTV | |
| | | SS (SEQ ID NO: 11) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNAGGSTYY | |
| | Variant 2 | ADSVKGRFTVSRDNSKNTLY | |
| | (QE, SA) | LQMNSLRAEDTALYYCAKDL | |
| | | GGYNYGLFDYWGQGTLVTV | |
| | | SS (SEQ ID NO: 12) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADSVKG | |
| | | (SEQ ID NO: 14)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNSGGSTYY | |
| | Variant 3 | ADAVKGRFTVSRDNSKNTLY | |
| | (QE, SA) | LQMNSLRAEDTALYYCAKDL | |
| | | GGYNYGLFDYWGQGTLVTV | |
| | | SS (SEQ ID NO: 15) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADAVKG | |
| | | (SEQ ID NO: 16)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNSGGSTYY | |
| | Variant | ADSVKGRFTVSRDNAKNTLY | |
| | 4(QE, SA) | LQMNSLRAEDTALYYCAKDL | |
| | | GGYNYGLFDYWGQGTLVTV | |
| | | SS (SEQ ID NO: 17) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNSGGSTYY | |
| | Variant 5 | ADSVKGRFTVSRDNSKQTLY | |
| | (QE, NQ) | LQMNSLRAEDTALYYCAKDL | |
| | | GGYNYGLFDYWGQGTLVTV | |
| | | SS (SEQ ID NO: 18) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNSGGSTYY | |
| | Variant 6 | ADSVKGRFTVSRDNSKNTLY | |
| | (QE, NQ) | LQMQSLRAEDTALYYCAKDL | |
| | | GGYNYGLFDYWGQGTLVTV | |
| | | SS (SEQ ID NO: 19) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNAGGSTYY | |
| | Variant 7 | ADAVKGRFTVSRDNSKNTLY | |
| | (QE, | LQMNSLRAEDTALYYCAKDL | |
| | SA, SA) | GGYNYGLFDYWGQGTLVTV | |
| | | SS (SEQ ID NO: 20) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNAGGSTYY | |
| | Variant 8 | ADAVKGRFTVSRDNAKNTLY | |
| | (QA, SA, | LQMNSLRAEDTALYYCAKDL | |
| | SA, SA) | GGYNYGLFDYWGQGTLVTV | |
| | | SS (SEQ ID NO: 22) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNAGGSTYY | |
| | Variant 9 | ADAVKGRFTVSRDNAKQTLY | |
| | (QE, SA, | LQMNSLRAEDTALYYCAKDL | |
| | SA, SA, | GGYNYGLFDYWGQGTLVTV | |
| | NQ) | SS (SEQ ID NO: 23) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNAGGSTYY | |
| | Variant 10 | ADAVKGRFTVSRDNAKQTLY | |
| | (QE, SA, | LQMQSLRAEDTALYYCAKDL | |
| | SA, SA, | GGYNYGLFDYWGQGTLVTV | |
| | NQ, NQ) | SS (SEQ ID NO: 24) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| | PH_420- | | DIRMTQSPSTLSASVGDRVTI |
| | CEACAM5_ | | TCWASQSISSWLAWYQQKP |
| | VL_ | | GKAPKLLIYKASSLESGVPS |
| | Variant 1 | | RFSGSGSGTEFTLTISSLQPD |
| | (DN) | | NFATYYCQQYNSYSYTFGQ |
| | | | GTKLEIK (SEQ ID NO: 25) |
| | | | CDR1: WASQSISSWLA (SEQ |
| | | | ID NO: 7) |
| | | | CDR2: KASSLES (SEQ ID |
| | | | NO: 8) |
| | | | CDR3: QQYNSYSYT (SEQ ID |
| | | | NO: 9) |
| | PH_420- | | DIRMTQSPSTLSASVGDRVTI |
| | CEACAM5_ | | TCWASQSISSWLAWYQQKP |
| | VL_ | | GKAPKLLIYKASSLESGVPS |
| | Variant 2 | | RFSGSGSGTEFTLTISSLQPD |
| | (SA) | | DFATYYCQQYNAYSYTFGQ |
| | | | GTKLEIK (SEQ ID NO: 26) |
| | | | CDR1: WASQSISSWLA (SEQ |
| | | | ID NO: 7) |
| | | | CDR2: KASSLES (SEQ ID |
| | | | NO: 8) |
| | | | CDR3: QQYNAYSYT (SEQ ID |
| | | | NO: 27) |
| | PH_420- | | DIRMTQSPSTLSASVGDRVTI |
| | CEACAM5_ | | TCWASQSISSWLAWYQQKP |
| | VL_ | | GKAPKLLIYKASSLESGVPS |
| | Variant 3 | | RFSGSGSGTEFTLTISSLQPD |
| | (DN, SA) | | NFATYYCQQYNAYSYTFGQ |
| | | | GTKLEIK (SEQ ID NO: 28) |
| | | | CDR1: WASQSISSWLA (SEQ |
| | | | ID NO: 7) |
| | | | CDR2: KASSLES (SEQ ID |
| | | | NO: 8) |
| | | | CDR3: QQYNAYSYT (SEQ ID |
| | | | NO: 27) |
| | PH_420- | | DIQMTQSPSTLSASVGDRVTI |
| | CEACAM5_ | | TCWASQSISSWLAWYQQKP |
| | VL_ | | GKAPKLLIYKASSLESGVPS |
| | Variant 4 | | RFSGSGSGTEFTLTISSLQPD |
| | (R(L3)Q) | | DFATYYCQQYNSYSYTFGQ |
| | | | GTKLEIK (SEQ ID NO: 637) |
| | | | CDR1: WASQSISSWLA (SEQ |
| | | | ID NO: 7) |
| | | | CDR2: KASSLES (SEQ ID |
| | | | NO: 8) |
| | | | CDR3: QQYNSYSYT (SEQ ID |
| | | | NO: 9) |
| 2. | Clone | X₁VQLVQSGGGLVQPGGSLR | DIX₁MTQSPSTLSASVGDRVT |
| | PH_420- | LSCAASGFTFSSYAMSWVRQ | ITCWASQSISSWLAWYQQKP |
| | CEACAM5 | APGKGLEWVSAIFNX₂GGSTY | GKAPKLLIYKASSLESGVPS |
| | and Variants | YADX₃VKGRFTVSRDNX₄KX₅ | RFSGSGSGTEFTLTISSLQPD |
| | Consensus | TLYLQMX₆SLRAEDTALYYC | X₂FATYYCQQYNX₃YSYTFG |
| | Sequence | AKDLGGYNYGLFDYWGQGT | QGTKLEIK |
| | | LVTVSS | where X₁ is R or Q, X₂ is N or |
| | | where X₁ is E or Q, X₂ is S or A, | D, and X₃ is S or A |
| | | X₃ is S or A, X₄ is S or A, X₅ is N | (SEQ ID NO: 32) |
| | | or Q, and X₆ is N or Q | CDR1: WASQSISSWLA (SEQ |
| | | (SEQ ID NO: 29) | ID NO: 7) |
| | | CDR1: GFTFSSY (SEQ ID | CDR2: KASSLES (SEQ ID |
| | | NO: 2)(Chothia) or SYAMS | NO: 8) |
| | | (SEQ ID NO: 3)(Kabat) | CDR3: QQYNXYSYT where X |
| | | CDR2: FNX₁GGS (SEQ ID | is S or A (SEQ ID NO: 33) |
| | | NO: 30)(Chothia) or | |
| | | AIFNX₁GGSTYYADX_{2V}KG | |
| | | (SEQ ID NO: 31)(Kabat) where | |
| | | X₁ is S or A and X₂ is S or A | |
| | | CDR3: DLGGYNYGLFDY | |
| | | (SEQ ID NO: 6) | |
| 3. | Clone | QVQLVQSGGGLVQPGGSQRL | DIQLTQSPATLSVSPGERATL |
| | 1078_C04CEACAM5_ | SCAASGFTFTSYAMSWVRQA | SCRASQSVSSSYLAWYQQK |
| | VH | PGKGLEWVSAISGTGDSTFYA | PGQAPRLLIYGASSRATGIPD |
| | | DSVKGRFTFSRDNSKNTLYL | RFSGSGSGTDFTLTISRLEPE |
| | | QMNSLRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 34) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFYADSVKG (SEQ | |
| | | ID NO: 37)(Kabat) | |
| | | CDR3: DLGWLQYGLFDY | |
| | | (SEQ ID NO: 38) | |
| 4. | 1078_C04CEACAM5_ | EVQLVQSGGGLVQPGGSQRL | DIQLTQSPATLSVSPGERATL |
| | VH_ | SCAASGFTFTSYAMSWVRQA | SCRASQSVSSSYLAWYQQK |
| | Variant 1 | PGKGLEWVSAISGTGDSTFYA | PGQAPRLLIYGASSRATGIPD |
| | (QE) | DSVKGRFTFSRDNSKNTLYL | RFSGSGSGTDFTLTISRLEPE |
| | | QMNSLRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 43) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFYADSVKG (SEQ | |
| | | ID NO: 37)(Kabat) | |
| | | CDR3: DLGWLQYGLFDY | |
| | | (SEQ ID NO: 38) | |
| 5. | 1078_C04CEACAM5_ | EVQLVQSGGGLVQPGGSQRL | DIQLTQSPATLSVSPGERATL |
| | VH_ | SCAASGFTFTSYAMSWVRQA | SCRASQSVSSSYLAWYQQK |
| | Variant 2 | PGKGLEWVSAISGTGESTFYA | PGQAPRLLIYGASSRATGIPD |
| | (QE, DE) | DSVKGRFTFSRDNSKNTLYL | RFSGSGSGTDFTLTISRLEPE |
| | | QMNSLRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 44) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGES (SEQ ID | CDR3: QQYNNWPLT (SEQ ID |
| | | NO: 45)(Chothia) or | NO: 42) |
| | | AISGTGESTFYADSVKG (SEQ | |
| | | ID NO: 46)(Kabat) | |
| | | CDR3: DLGWLQYGLFDY | |
| | | (SEQ ID NO: 38) | |
| 6. | 1078_C04CEACAM5_ | EVQLVQSGGGLVQPGGSQRL | DIQLTQSPATLSVSPGERATL |
| | VH_ | SCAASGFTFTSYAMSWVRQA | SCRASQSVSSSYLAWYQQK |
| | Variant 3 | PGKGLEWVSAISGTGDSTFYA | PGQAPRLLIYGASSRATGIPD |
| | (QE, SA) | DAVKGRFTFSRDNSKNTLYL | RFSGSGSGTDFTLTISRLEPE |
| | | QMNSLRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 47) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFYADAVKG (SEQ | |
| | | ID NO: 48)(Kabat) | |
| | | CDR3: DLGWLQYGLFDY | |
| | | (SEQ ID NO: 38) | |
| 7. | 1078_C04CEACAM5_ | EVQLVQSGGGLVQPGGSQRL | DIQLTQSPATLSVSPGERATL |
| | VH_ | SCAASGFTFTSYAMSWVRQA | SCRASQSVSSSYLAWYQQK |
| | Variant 4 | PGKGLEWVSAISGTGDSTFYA | PGQAPRLLIYGASSRATGIPD |
| | (QE, DE) | DSVKGRFTFSRENSKNTLYLQ | RFSGSGSGTDFTLTISRLEPE |
| | | MNSLRAEDTAVYYCAKDLG | DFAVYYCQQYNNWPLTFGG |
| | | WLQYGLFDYWGQGTLVTVS | GTKVEIK (SEQ ID NO: 39) |
| | | S (SEQ ID NO: 49) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFYADSVKG (SEQ | |
| | | ID NO: 37)(Kabat) | |
| | | CDR3: DLGWLQYGLFDY | |
| | | (SEQ ID NO: 38) | |
| 8. | 1078_C04CEACAM5_ | QVQLVQSGGGLVQPGGSQRL | DIQLTQSPATLSVSPGERATL |
| | VH_ | SCAASGFTFTSYAMSWVRQA | SCRASQSVSSSYLAWYQQK |
| | Variant 5 | PGKGLEWVSAISGTGDSTFYA | PGQAPRLLIYGASSRATGIPD |
| | (QE, SA) | DSVKGRFTFSRDNAKNTLYL | RFSGSGSGTDFTLTISRLEPE |
| | | QMNSLRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 50) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFYADSVKG (SEQ | |
| | | ID NO: 37)(Kabat) | |
| | | CDR3: DLGWLQYGLFDY | |
| | | (SEQ ID NO: 38) | |
| 9. | 1078_C04CEACAM5_ | EVQLVQSGGGLVQPGGSQRL | DIQLTQSPATLSVSPGERATL |
| | VH_ | SCAASGFTFTSYAMSWVRQA | SCRASQSVSSSYLAWYQQK |
| | Variant 6 | PGKGLEWVSAISGTGDSTFYA | PGQAPRLLIYGASSRATGIPD |
| | (QE, SA) | DSVKGRFTFSRDNSKNTLYL | RFSGSGSGTDFTLTISRLEPE |
| | | QMNALRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 51) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFY ADSVKG (SEQ | |
| | | ID NO: 37)(Kabat) | |
| | | CDR3: DLGWLQYGLFDY | |
| | | (SEQ ID NO: 38) | |
| 10. | 1078_C04CEACAM5_ | QVQLVQSGGGLVQPGGSLRL | DIQLTQSPATLSVSPGERATL |
| | VH_ | SCAASGFTFTSYAMSWVRQA | SCRASQSVSSSYLAWYQQK |
| | Variant 7 | PGKGLEWVSAISGTGDSTFYA | PGQAPRLLIYGASSRATGIPD |
| | (Q(H18)L) | DSVKGRFTFSRDNSKNTLYL | RFSGSGSGTDFTLTISRLEPE |
| | | QMNSLRAEDTAVYYCAKDL | DFAVYYCQQYNNWPLTFGG |
| | | GWLQYGLFDYWGQGTLVTV | GTKVEIK (SEQ ID NO: 39) |
| | | SS (SEQ ID NO: 638) | CDR1: RASQSVSSSYLA (SEQ |
| | | CDR1: GFTFTSY (SEQ ID | ID NO: 40) |
| | | NO: 35)(Chothia) or SYAMS | CDR2: GASSRAT (SEQ ID |
| | | (SEQ ID NO: 3)(Kabat) | NO: 41) |
| | | CDR2: SGTGDS (SEQ ID | CDR3: QQYNNWPLT (SEQ ID |
| | | NO: 36)(Chothia) or | NO: 42) |
| | | AISGTGDSTFYADSVKG (SEQ | |
| | | ID NO: 37)(Kabat) | |
| | | CDR3: DLGWLQYGLFDY | |
| | | (SEQ ID NO: 38) | |
| 11. | 1078_C04CEACAM5 | X₁VQLVQSGGGLVQPGGSX₂R | DIQLTQSPATLSVSPGERATL |
| | and Variants | LSCAASGFTFTSYAMSWVRQ | SCRASQSVSSSYLAWYQQK |
| | Consensus | APGKGLEWVSAISGTGX₃STF | PGQAPRLLIYGASSRATGIPD |
| | Sequence | YADX₄VKGRFTFSRX₅NX₆KN | RFSGSGSGTDFTLTISRLEPE |
| | | TLYLQMNX₇LRAEDTAVYYC | DFAVYYCQQYNNWPLTFGG |
| | | AKDLGWLQYGLFDYWGQGT | GTKVEIK (SEQ ID NO: 39) |
| | | LVTVSS | CDR1: RASQSVSSSYLA (SEQ |
| | | where X₁ is E or Q, X₂ is Q or L, | ID NO: 40) |
| | | X₃ is D or E, X₄ is S or A, X₅ is D | CDR2: GASSRAT (SEQ ID |
| | | or E, X₆ is S or A, and X₇ is S or | NO: 41) |
| | | A | CDR3: QQYNNWPLT (SEQ ID |
| | | (SEQ ID NO: 52) | NO: 42) |
| | | CDR1: GFTFTSY (SEQ ID | |
| | | NO: 35)(Chothia) or SYAMS | |
| | | (SEQ ID NO: 3)(Kabat) | |
| | | CDR2: SGTGX₁S (SEQ ID | |
| | | NO: 53)(Chothia) or | |
| | | AISGTGX₁STFYADX₂VKG | |
| | | (SEQ ID NO: 54)(Kabat) where | |
| | | X₁ is D or E and X₂ is S or A | |
| | | CDR3: DLGWLQYGLFDY | |
| | | (SEQ ID NO: 38) | |
| 12. | Clone | QVQLQESGPGLVRPSGTLSLT | DIVMTQTPATLSASVGDRVT |
| | 1079_H05CEACAM5_ | CAVSGGSISSPTWWSWVRQP | ITCRASQSVRSNLAWYQQKP |
| | VH | PGKGLEWIGEIHPSGRTNYNP | GQAPRLLIYGASTRATGIPA |
| | | SLKSRVTISVDKSKNQFSLKL | RFSGSGSGTEFTLTISSLQSE |
| | | GSVTAADTAVYYCAREGFYY | DFAVYYCQQYNNWPTFGQ |
| | | GSGNYYYFDYWGQGTLVTV | GTRLEIK (SEQ ID NO: 61) |
| | | SS (SEQ ID NO: 55) | CDR1: RASQSVRSNLA (SEQ |
| | | CDR1: GGSISSPT (SEQ ID | ID NO: 62) |
| | | NO: 56)(Chothia) or SPTWWS | CDR2: GASTRAT (SEQ ID |
| | | (SEQ ID NO: 57)(Kabat) | NO: 63) |
| | | CDR2: HPSGR (SEQ ID NO: 58) | CDR3: QQYNNWPT (SEQ ID |
| | | (Chothia) or | NO: 64) |
| | | EIHPSGRTNYNPSLKS (SEQ ID | |
| | | NO: 59)(Kabat) | |
| | | CDR3: EGFYYGSGNYYYFDY | |
| | | (SEQ ID NO: 60) | |
| 13. | 1079_H05CEACAM5_ | EVQLQESGPGLVRPSGTLSLT | DIVMTQTPATLSASVGDRVT |
| | VH_ | CAVSGGSISSPTWWSWVRQP | ITCRASQSVRSNLAWYQQKP |
| | Variant 1 | PGKGLEWIGEIHPSGRTNYNP | GQAPRLLIYGASTRATGIPA |
| | (QE) | SLKSRVTISVDKSKNQFSLKL | RFSGSGSGTEFTLTISSLQSE |
| | | GSVTAADTAVYYCAREGFYY | DFAVYYCQQYNNWPTFGQ |
| | | GSGNYYYFDYWGQGTLVTV | GTRLEIK (SEQ ID NO: 61) |
| | | SS (SEQ ID NO: 65) | CDR1: RASQSVRSNLA (SEQ |
| | | CDR1: GGSISSPT (SEQ ID | ID NO: 62) |
| | | NO: 56)(Chothia) or SPTWWS | CDR2: GASTRAT (SEQ ID |
| | | (SEQ ID NO: 57)(Kabat) | NO: 63) |
| | | CDR2: HPSGR (SEQ ID NO: 58) | CDR3: QQYNNWPT (SEQ ID |
| | | (Chothia) or | NO: 64) |
| | | EIHPSGRTNYNPSLKS (SEQ ID | |
| | | NO: 59)(Kabat) | |
| | | CDR3: EGFYYGSGNYYYFDY | |
| | | (SEQ ID NO: 60) | |
| 14. | 1079_H05- | XVQLQESGPGLVRPSGTLSLT | DIVMTQTPATLSASVGDRVT |
| | CEACAM5 | CAVSGGSISSPTWWSWVRQP | ITCRASQSVRSNLAWYQQKP |
| | and Variants | PGKGLEWIGEIHPSGRTNYNP | GQAPRLLIYGASTRATGIPA |
| | Consensus | SLKSRVTISVDKSKNQFSLKL | RFSGSGSGTEFTLTISSLQSE |
| | Sequence | GSVTAADTAVYYCAREGFYY | DFAVYYCQQYNNWPTFGQ |
| | | GSGNYYYFDYWGQGTLVTV | GTRLEIK (SEQ ID NO: 61) |
| | | SS | CDR1: RASQSVRSNLA (SEQ |
| | | where X is Q or E | ID NO: 62) |
| | | (SEQ ID NO: 66) | CDR2: GASTRAT (SEQ ID |
| | | CDR1: GGSISSPT (SEQ ID | NO: 63) |
| | | NO: 56)(Chothia) or SPTWWS | CDR3: QQYNNWPT (SEQ ID |
| | | (SEQ ID NO: 57)(Kabat) | NO: 64) |
| | | CDR2: HPSGR (SEQ ID NO: 58) | |
| | | (Chothia) or | |
| | | EIHPSGRTNYNPSLKS (SEQ ID | |
| | | NO: 59)(Kabat) | |
| | | CDR3: EGFYYGSGNYYYFDY | |
| | | (SEQ ID NO: 60) | |
| 15. | 7A10.A7- | QVQLVESGGDVVQPGRSLRL | EIVMTQSPLSLPVTPGEPASI |
| | CEACAM5- | SCAASGFILSNYGMHWVRQA | SCRSSQSLLHSNGYNYLDW |
| | B.01 | PGKGLEWVAAMWYDGSNN | YLQKPGQSPQLLISLGSIRAS |
| | | YYEDSVKGRFTISRDNSKNTL | GVPDRFSGSGSGTNFTLTISR |
| | | YLQMNSLRAEDTAVYYCARE | VEAEDVGFYYCMQALQTPR |
| | | RVSRHFDWHYYYGMDVWG | TFGQGTKVDIT (SEQ ID |
| | | QGTTVTVSS (SEQ ID NO: 67) | NO: 73) |
| | | CDR1: GFILSNY (SEQ ID | CDR1: |
| | | NO: 68)(Chothia) or NYGMH | RSSQSLLHSNGYNYLD (SEQ |
| | | (SEQ ID NO: 69)(Kabat) | ID NO: 74) |
| | | CDR2: WYDGSN (SEQ ID | CDR2: LGSIRAS (SEQ ID |
| | | NO: 70)(Chothia) or | NO: 75) |
| | | AMWYDGSNNYYEDSVKG | CDR3: MQALQTPRT (SEQ ID |
| | | (SEQ ID NO: 71)(Kabat) | NO: 76) |
| | | CDR3: | |
| | | ERVSRHFDWHYYYGMDV | |
| | | (SEQ ID NO: 72) | |
| | 7A10.A7- | EVQLVESGGDVVQPGRSLRL | |
| | CEACAM5- | SCAASGFILSNYGMHWVRQA | |
| | B.01_ | PGKGLEWVAAMWYDGSNN | |
| | VH_ | YYEDSVKGRFTISRDNSKNTL | |
| | Variant | YLQMNSLRAEDTAVYYCARE | |
| | 1_(QE) | RVSRHFDWHYYYGMDVWG | |
| | | QGTTVTVSS (SEQ ID NO: 77) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | 7A10.A7- | EVQLVESGGDVVQPGRSLRL | |
| | CEACAM5- | SCAASGFILSNYGMHWVRQA | |
| | B.01_ | PGKGLEWVAAMWYDGSNN | |
| | VH_ | YYEDTVKGRFTISRDNSKNTL | |
| | Variant | YLQMNSLRAEDTAVYYCARE | |
| | 2_(QE, ST) | RVSRHFDWHYYYGMDVWG | |
| | | QGTTVTVSS (SEQ ID NO: 78) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDTVKG | |
| | | (SEQ ID NO: 79)(Kabat) | |
| | | CDR3: | |
| | | ERVSRHFDWHYYYGMDV | |
| | | (SEQ ID NO: 72) | |
| | 7A10.A7- | EVQLVESGGDVVQPGRSLRL | |
| | CEACAM5- | SCAASGFILSNYGMHWVRQA | |
| | B.01_ | PGKGLEWVAAMWYDGSNN | |
| | VH_ | YYEDSVKGRFTISRDQSKNTL | |
| | Variant | YLQMNSLRAEDTAVYYCARE | |
| | 3_(QE, NQ) | RVSRHFDWHYYYGMDVWG | |
| | | QGTTVTVSS (SEQ ID NO: 80) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | 7A10.A7- | EVQLVESGGDVVQPGRSLRL | |
| | CEACAM5- | SCAASGFILSNYGMHWVRQA | |
| | B.01_ | PGKGLEWVAAMWYDGSNN | |
| | VH_ | YYEDSVKGRFTISRDNSKQTL | |
| | Variant | YLQMNSLRAEDTAVYYCARE | |
| | 4_(QE, NQ) | RVSRHFDWHYYYGMDVWG | |
| | | QGTTVTVSS (SEQ ID NO: 81) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | 7A10.A7- | QVQLVESGGDVVQPGRSLRL | |
| | CEACAM5- | SCAASGFILSNYGMHWVRQA | |
| | B.01_ | PGKGLEWVAAMWYDGSNN | |
| | VH_ | YYEDSVKGRFTISRDNSKNTL | |
| | Variant | YLQMQSLRAEDTAVYYCARE | |
| | 5_(QE, NQ) | RVSRHFDWHYYYGMDVWG | |
| | | QGTTVTVSS (SEQ ID NO: 82) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | 7A10.A7- | EVQLVESGGDVVQPGRSLRL | |
| | CEACAM5- | SCAASGFILSNYGMHWVRQA | |
| | B.01_ | PGKGLEWVAAMWYDGSNN | |
| | VH_ | YYEDTVKGRFTISRDQSKQTL | |
| | Variant | YLQMQSLRAEDTAVYYCARE | |
| | 6_(QE, ST, | RVSRHFDWHYYYGMDVWG | |
| | NQ, NQ, | QGTTVTVSS (SEQ ID NO: 83) | |
| | NQ) | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDTVKG | |
| | | (SEQ ID NO: 79)(Kabat) | |
| | | CDR3: | |
| | | ERVSRHFDWHYYYGMDV | |
| | | (SEQ ID NO: 72) | |
| | 7A10.A7- | | EIVMTQSPLSLPVTPGEPASI |
| | CEACAM5- | | SCRSSQSLLHSNGYNYLDW |
| | B.01- | | YLQKPGQSPQLLISLGSIRAS |
| | N(L70)D | | GVPDRFSGSGSGTDFTLTISR |
| | VL | | VEAEDVGFYYCMQALQTPR |
| | | | TFGQGTKVDIT (SEQ ID |
| | | | NO: 84) |
| | | | CDR1: |
| | | | RSSQSLLHSNGYNYLD (SEQ |
| | | | ID NO: 74) |
| | | | CDR2: LGSIRAS (SEQ ID |
| | | | NO: 75) |
| | | | CDR3: MQALQTPRT (SEQ ID |
| | | | NO: 76) |
| | 7A10.A7- | | EIVMTQSPLSLPVTPGEPASI |
| | CEACAM5- | | SCRSSQSLLHSNGYNYLDW |
| | B.01- | | YLQKPGQSPQLLISLGSIRAS |
| | N(L70)Q | | GVPDRFSGSGSGTQFTLTISR |
| | VL | | VEAEDVGFYYCMQALQTPR |
| | | | TFGQGTKVDIT (SEQ ID |
| | | | NO: 85) |
| | | | CDR1: |
| | | | RSSQSLLHSNGYNYLD (SEQ |
| | | | ID NO: 74) |
| | | | CDR2: LGSIRAS (SEQ ID |
| | | | NO: 75) |
| | | | CDR3: MQALQTPRT (SEQ ID |
| | | | NO: 76) |
| | 7A10.A7- | | EIVMTQSPLSLPVTPGEPASI |
| | CEACAM5- | | SCRSSQSLLHSNGYNYLDW |
| | B.01_ | | YLQKPGQSPQLLISLGSIRAS |
| | VL_ | | GVPDRFSGSGSGTNFALTISR |
| | Variant | | VEAEDVGFYYCMQALQTPR |
| | 1_(TA) | | TFGQGTKVDIT (SEQ ID |
| | | | NO: 86) |
| | | | CDR1: |
| | | | RSSQSLLHSNGYNYLD (SEQ |
| | | | ID NO: 74) |
| | | | CDR2: LGSIRAS (SEQ ID |
| | | | NO: 75) |
| | | | CDR3: MQALQTPRT (SEQ ID |
| | | | NO: 76) |
| 16. | 7A10.A7- | X₁VQLVESGGDVVQPGRSLRL | EIVMTQSPLSLPVTPGEPASI |
| | CEACAM5- | SCAASGFILSNYGMHWVRQA | SCRSSQSLLHSNGYNYLDW |
| | B.01 | PGKGLEWVAAMWYDGSNN | YLQKPGQSPQLLISLGSIRAS |
| | and Variants | YYEDX₂VKGRFTISRDX₃SKX₄ | GVPDRFSGSGSGTX₁FX₂LTIS |
| | Consensus | TLYLQMX₅SLRAEDTAVYYC | RVEAEDVGFYYCMQALQTP |
| | Sequence | ARERVSRHFDWHYYYGMDV | RTFGQGTKVDIT |
| | | WGQGTTVTVSS | where X₁ is N, D or Q and X₂ is |
| | | where X₁ is E or Q, X₂ is S or T, | T or A |
| | | X₃ is N or Q, X₄ is N or Q, and | (SEQ ID NO: 89) |
| | | X₅ is N or Q | CDR1: |
| | | (SEQ ID NO: 87) | RSSQSLLHSNGYNYLD (SEQ |
| | | CDR1: GFILSNY (SEQ ID | ID NO: 74) |
| | | NO: 68)(Chothia) or NYGMH | CDR2: LGSIRAS (SEQ ID |
| | | (SEQ ID NO: 69)(Kabat) | NO: 75) |
| | | CDR2: WYDGSN (SEQ ID | CDR3: MQALQTPRT (SEQ ID |
| | | NO: 70)(Chothia) or | NO: 76) |
| | | AMWYDGSNNYYEDXVKG | |
| | | (Kabat) where X is S or T (SEQ | |
| | | ID NO: 88) | |
| | | CDR3: | |
| | | ERVSRHFDWHYYYGMDV | |
| | | (SEQ ID NO: 72) | |
| | 7A10.A7- | | EIVMTQSPLSLPVTPGEPASI |
| | CEACAM5- | | SCRSSQSLLHSSGYNYLDWY |
| | B.01- | | LQKPGQSPQLLISLGSIRASG |
| | N(L70)D, N | | VPDRFSGSGSGTDFTLTISRV |
| | (L28)S VL | | EAEDVGFYYCMQALQTPRT |
| | | | FGQGTKVDIT (SEQ ID |
| | | | NO: 631) |
| | | | CDR1: |
| | | | RSSQSLLHSSGYNYLD (SEQ |
| | | | ID NO: 632) |
| | | | CDR2: LGSIRAS (SEQ ID |
| | | | NO: 75) |
| | | | CDR3: MQALQTPRT (SEQ ID |
| | | | NO: 76) |
| | 7A10.A7- | | EIVMTQSPLSLPVTPGEPASI |
| | CEACAM5- | | SCRSSQSLLHSQGYNYLDW |
| | B.01- | | YLQKPGQSPQLLISLGSIRAS |
| | N(L70)D, N | | GVPDRFSGSGSGTDFTLTISR |
| | (L28)Q VL | | VEAEDVGFYYCMQALQTPR |
| | | | TFGQGTKVDIT (SEQ ID |
| | | | NO: 633) |
| | | | CDR1: |
| | | | RSSQSLLHSQGYNYLD (SEQ |
| | | | ID NO: 634) |
| | | | CDR2: LGSIRAS (SEQ ID |
| | | | NO: 75) |
| | | | CDR3: MQALQTPRT (SEQ ID |
| | | | NO: 76) |
| | 7A10.A7- | | EIVMTQSPLSLPVTPGEPASI |
| | CEACAM5- | | SCRSSQSLLHSNAYNYLDW |
| | B.01- | | YLQKPGQSPQLLISLGSIRAS |
| | N(L70)D, G | | GVPDRFSGSGSGTDFTLTISR |
| | (L29)A VL | | VEAEDVGFYYCMQALQTPR |
| | | | TFGQGTKVDIT (SEQ ID |
| | | | NO: 635) |
| | | | CDR1: |
| | | | RSSQSLLHSNAYNYLD (SEQ |
| | | | ID NO: 636) |
| | | | CDR2: LGSIRAS (SEQ ID |
| | | | NO: 75) |
| | | | CDR3: MQALQTPRT (SEQ ID |
| | | | NO: 76) |
| 17. | 8H2.B10- | QVQLVESGGDVVQPGRSLRL | EIVMTQSPLSLPVTPGEPASI |
| | CEACAM5- | SCAASGFTLSSYGMHWVRQA | SCRSSQSLLHYNGYNYLDW |
| | B.01 | PGKGLEWVAAMWYDGSNN | YLQKPGQSPQLLISLGSIRAS |
| | | YYEDSVKGRFTISRDNSKNTL | GVPDRFSGSGSGTNFTLTISR |
| | | YLQMNSLRAEDTAVYYCARE | VEAEDVGFYYCMQALQTPR |
| | | RVSRHFDWHYYYGMDVWG | TFGQGTKVDIT (SEQ ID |
| | | QGTTVTVSS (SEQ ID NO: 90) | NO: 93) |
| | | CDR1: GFTLSSY (SEQ ID | CDR1: |
| | | NO: 91)(Chothia) or SYGMH | RSSQSLLHYNGYNYLD (SEQ |
| | | (SEQ ID NO: 92)(Kabat) | ID NO: 94) |
| | | CDR2: WYDGSN (SEQ ID | CDR2: LGSIRAS (SEQ ID |
| | | NO: 70)(Chothia) or | NO: 75) |
| | | AMWYDGSNNYYEDSVKG | CDR3: MQALQTPRT (SEQ ID |
| | | (SEQ ID NO: 71)(Kabat) | NO: 76) |
| | | CDR3: | |
| | | ERVSRHFDWHYYYGMDV | |
| | | (SEQ ID NO: 72) | |
| | 8H2.B10- | | EIVMTQSPLSLPVTPGEPASI |
| | CEACAM5- | | SCRSSQSLLHYNGYNYLDW |
| | B.01- | | YLQKPGQSPQLLISLGSIRAS |
| | N(L70)D | | GVPDRFSGSGSGTDFTLTISR |
| | VL | | VEAEDVGFYYCMQALQTPR |
| | | | TFGQGTKVDIT (SEQ ID |
| | | | NO: 95) |
| | | | CDR1: |
| | | | RSSQSLLHYNGYNYLD (SEQ |
| | | | ID NO: 94) |
| | | | CDR2: LGSIRAS (SEQ ID |
| | | | NO: 75) |
| | | | CDR3: MQALQTPRT (SEQ ID |
| | | | NO: 76) |
| | 8H2.B10- | | EIVMTQSPLSLPVTPGEPASI |
| | CEACAM5- | | SCRSSQSLLHYNGYNYLDW |
| | B.01- | | YLQKPGQSPQLLISLGSIRAS |
| | N(L70)Q | | GVPDRFSGSGSGTQFTLTISR |
| | VL | | VEAEDVGFYYCMQALQTPR |
| | | | TFGQGTKVDIT (SEQ ID |
| | | | NO: 96) |
| | | | CDR1: |
| | | | RSSQSLLHYNGYNYLD (SEQ |
| | | | ID NO: 94) |
| | | | CDR2: LGSIRAS (SEQ ID |
| | | | NO: 75) |
| | | | CDR3: MQALQTPRT (SEQ ID |
| | | | NO: 76) |
| | 8H2.B10- | | EIVMTQSPLSLPVTPGEPASI |
| | CEACAM5- | | SCRSSQSLLHYNGYNYLDW |
| | B.01- | | YLQKPGQSPQLLISLGSIRAS |
| | T(L72)A | | GVPDRFSGSGSGTNFALTISR |
| | VL | | VEAEDVGFYYCMQALQTPR |
| | | | TFGQGTKVDIT (SEQ ID |
| | | | NO: 97) |
| | | | CDR1: |
| | | | RSSQSLLHYNGYNYLD (SEQ |
| | | | ID NO: 94) |
| | | | CDR2: LGSIRAS (SEQ ID |
| | | | NO: 75) |
| | | | CDR3: MQALQTPRT (SEQ ID |
| | | | NO: 76) |
| | VH | EVQLVESGGDVVQPGRSLRL | |
| | 8H2.B10- | SCAASGFTLSSYGMHWVRQA | |
| | CEACAM5- | PGKGLEWVAAMWYDGSNN | |
| | B.01 | YYEDSVKGRFTISRDNSKNTL | |
| | (QH1E) | YLQMNSLRAEDTAVYYCARE | |
| | | RVSRHFDWHYYYGMDVWG | |
| | | QGTTVTVSS (SEQ ID NO: 332) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | VH | QVQLVESGGDVVQPGRSLRL | |
| | 8H2.B10- | SCAASGFTLSSYGMHWVRQA | |
| | CEACAM5- | PGKGLEWVAAMWYDGSNN | |
| | B.01 | YYEDTVKGRFTISRDNSKNTL | |
| | (SH62T) | YLQMNSLRAEDTAVYYCARE | |
| | | RVSRHFDWHYYYGMDVWG | |
| | | QGTTVTVSS (SEQ ID NO: 333) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDTVKG | |
| | | (SEQ ID NO: 79)(Kabat) | |
| | | CDR3: | |
| | | ERVSRHFDWHYYYGMDV | |
| | | (SEQ ID NO: 72) | |
| | VH_8H2.B10- | QVQLVESGGDVVQPGRSLRL | |
| | CEACAM5- | SCAASGFTLSSYGMHWVRQA | |
| | B.01 | PGKGLEWVAAMWYDGSNN | |
| | (NH73Q) | YYEDSVKGRFTISRDQSKNTL | |
| | | YLQMNSLRAEDTAVYYCARE | |
| | | RVSRHFDWHYYYGMDVWG | |
| | | QGTTVTVSS (SEQ ID NO: 334) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | VH 8H2.B10- | QVQLVESGGDVVQPGRSLRL | |
| | CEACAM5- | SCAASGFTLSSYGMHWVRQA | |
| | B.01 | PGKGLEWVAAMWYDGSNN | |
| | (NH76Q) | YYEDSVKGRFTISRDNSKQTL | |
| | | YLQMNSLRAEDTAVYYCARE | |
| | | RVSRHFDWHYYYGMDVWG | |
| | | QGTTVTVSS (SEQ ID NO: 335) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| | VH 8H2.B10- | QVQLVESGGDVVQPGRSLRL | |
| | CEACAM5- | SCAASGFTLSSYGMHWVRQA | |
| | B.01 | PGKGLEWVAAMWYDGSNN | |
| | (NH82AQ) | YYEDSVKGRFTISRDNSKNTL | |
| | | YLQMQSLRAEDTAVYYCARE | |
| | | RVSRHFDWHYYYGMDVWG | |
| | | QGTTVTVSS (SEQ ID NO: 336) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 18. | 8H2.B10- | X₁VQLVESGGDVVQPGRSLRL | EIVMTQSPLSLPVTPGEPASI |
| | CEACAM5- | SCAASGFTLSSYGMHWVRQA | SCRSSQSLLHYNGYNYLDW |
| | B.01 | PGKGLEWVAAMWYDGSNN | YLQKPGQSPQLLISLGSIRAS |
| | and Variants | YYEDX₂VKGRFTISRDX₃SKX₄ | GVPDRFSGSGSGTX₁FX₂LTIS |
| | Consensus | TLYLQMX₅SLRAEDTAVYYC | RVEAEDVGFYYCMQALQTP |
| | Sequence | ARERVSRHFDWHYYYGMDV | RTFGQGTKVDIT |
| | | WGQGTTVTVSS | where X₁ is N, D or Q and X₂ is |
| | | where X₁ is E or Q, X₂ is S or T, | A or T |
| | | X₃ is N or Q, X₄ is N or Q, and | (SEQ ID NO: 99) |
| | | X₅ is N or Q | CDR1: |
| | | (SEQ ID NO: 98) | RSSQSLLHYNGYNYLD (SEQ |
| | | CDR1: GFTLSSY (SEQ ID | ID NO: 94) |
| | | NO: 91)(Chothia) or SYGMH | CDR2: LGSIRAS (SEQ ID |
| | | (SEQ ID NO: 92)(Kabat) | NO: 75) |
| | | CDR2: WYDGSN (SEQ ID | CDR3: MQALQTPRT (SEQ ID |
| | | NO: 70)(Chothia) or | NO: 76) |
| | | AMWYDGSNNYYEDXVKG | |
| | | (SEQ ID NO: 88)(Kabat) where | |
| | | X is S or T | |
| | | CDR3: | |
| | | ERVSRHFDWHYYYGMDV | |
| | | (SEQ ID NO: 72) | |
| 19. | Murine | EVQLQQSAAELARPGASVKM | DIVMSQSPSSLAVSVGEKVT |
| | 16F6.A2- | SCKASGYTFTAYTIHWVKQR | MSCKSSHSLLYGNFQNNYL |
| | CEACAM5- | PGQGLEWIGYINPSSGYTEYN | AWYQQKPGQSPKLLIYWAS |
| | B.02 (VH | QKFKDKTTLTADQSSPTAYIQ | TRESGVPDRFTGSGSGTDFT |
| | Variant 1) | LSTLTSEDSAVYYCTREGGLL | LTISSVKAEDLAVYYCQQYY |
| | | WFDYWGQGTTLTVST (SEQ | SYPYTFGGGTKLEIK (SEQ |
| | | ID NO: 100) | ID NO: 106) |
| | | CDR1: GYTFTAY (SEQ ID | CDR1: |
| | | NO: 101)(Chothia) or AYTIH | KSSHSLLYGNFQNNYLA |
| | | (SEQ ID NO: 102)(Kabat) | (SEQ ID NO: 107) |
| | | CDR2: NPSSGY (SEQ ID | CDR2: WASTRES (SEQ ID |
| | | NO: 103)(Chothia) or | NO: 108) |
| | | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 20. | Murine | QVQLQQSAAELARPGASVKM | DIVMSQSPSSLAVSVGEKVT |
| | 16F6.A2- | SCKASGYTFTAYTIHWVKQR | MSCKSSHSLLYGNFQNNYL |
| | CEACAM5- | PGQGLEWIGYINPSSGYTEYN | AWYQQKPGQSPKLLIYWAS |
| | B.02 (VH | QKFKDKTTLTADQSSPTAYIQ | TRESGVPDRFTGSGSGTDFT |
| | Variant 2) | LSTLTSEDSAVYYCTREGGLL | LTISSVKAEDLAVYYCQQYY |
| | | WFDYWGQGTTLTVST (SEQ | SYPYTFGGGTKLEIK (SEQ |
| | | ID NO: 110) | ID NO: 106) |
| | | CDR1: GYTFTAY (SEQ ID | CDR1: |
| | | NO: 101)(Chothia) or AYTIH | KSSHSLLYGNFQNNYLA |
| | | (SEQ ID NO: 102)(Kabat) | (SEQ ID NO: 107) |
| | | CDR2: NPSSGY (SEQ ID | CDR2: WASTRES (SEQ ID |
| | | NO: 103)(Chothia) or | NO: 108) |
| | | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 21. | Murine | XVQLQQSAAELARPGASVKM | DIVMSQSPSSLAVSVGEKVT |
| | 16F6.A2- | SCKASGYTFTAYTIHWVKQR | MSCKSSHSLLYGNFQNNYL |
| | CEACAM5- | PGQGLEWIGYINPSSGYTEYN | AWYQQKPGQSPKLLIYWAS |
| | B.02 | QKFKDKTTLTADQSSPTAYIQ | TRESGVPDRFTGSGSGTDFT |
| | Consensus | LSTLTSEDSAVYYCTREGGLL | LTISSVKAEDLAVYYCQQYY |
| | Sequence | WFDYWGQGTTLTVST | SYPYTFGGGTKLEIK (SEQ |
| | | where X is E or Q | ID NO: 106) |
| | | (SEQ ID NO: 111) | CDR1: |
| | | CDR1: GYTFTAY (SEQ ID | KSSHSLLYGNFQNNYLA |
| | | NO: 101)(Chothia) or AYTIH | (SEQ ID NO: 107) |
| | | (SEQ ID NO: 102)(Kabat) | CDR2: WASTRES (SEQ ID |
| | | CDR2: NPSSGY (SEQ ID | NO: 108) |
| | | NO: 103)(Chothia) or | CDR3: QQYYSYPYT (SEQ ID |
| | | YINPSSGYTEYNQKFKD (SEQ | NO: 109) |
| | | ID NO: 104)(Kabat) | |
| | | CDR3: EGGLLWFDY (SEQ ID | |
| | | NO: 105) | |
| 22. | Humanized | QVQLVQSGAEVKKPGASVK | DIVMTQSPDSLAVSLGERAT |
| | 16F6.A2- | MSCKASGYTFTAYTIHWVRQ | INCKSSHSLLYGNFQNNYLA |
| | CEACAM5- | APGQGLEWIGYINPSSGYTEY | WYQQKPGQPPKLLIYWAST |
| | B.02-BM- | NQKFKDRTTLTADTSIPTAYM | RESGVPDRFSGSGSGTDFTL |
| | H1-VH | ELSRLRSDDTAVYYCTREGG | TISSLQAEDVAVYYCQQYYS |
| | and | LLWFDYWGQGTLVTVSS | YPYTFGGGTKLEIK (SEQ ID |
| | Humanized | (SEQ ID NO: 112) | NO: 119) |
| | 16F6.A2- | CDR1: GYTFTAY (SEQ ID | CDR1: |
| | CEACAM5- | NO: 101)(Chothia) or AYTIH | KSSHSLLYGNFQNNYLA |
| | B.02-BM- | (SEQ ID NO: 102)(Kabat) | (SEQ ID NO: 107) |
| | L1-VL | CDR2: NPSSGY (SEQ ID | CDR2: WASTRES (SEQ ID |
| | | NO: 103)(Chothia) or | NO: 108) |
| | | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 23. | Humanized | QVQLVQSGAEVKKPGASVK | DIVMTQSPDSLAVSLGERAT |
| | 16F6.A2- | MSCKASGYTFTAYTIHWVRQ | INCKSSHSLLYGNFQNNYLA |
| | CEACAM5- | APGQGLEWIGYINPSSGYTEY | WYQQKPGQPPKLLIYWAST |
| | B.02-BM- | NQKFKDRTTLTADTSIPTAYI | RESGVPDRFSGSGSGTDFTL |
| | H2-VH and | ELSRLRSDDTAVYYCTREGG | TISSLQAEDVAVYYCQQYYS |
| | Humanized | LLWFDYWGQGTLVTVSS | YPYTFGGGTKLEIK (SEQ ID |
| | 16F6.A2- | (SEQ ID NO: 113) | NO: 119) |
| | CEACAM5- | CDR1: GYTFTAY (SEQ ID | CDR1: |
| | B.02-BM- | NO: 101)(Chothia) or AYTIH | KSSHSLLYGNFQNNYLA |
| | L1-VL | (SEQ ID NO: 102)(Kabat) | (SEQ ID NO: 107) |
| | | CDR2: NPSSGY (SEQ ID | CDR2: WASTRES (SEQ ID |
| | | NO: 103)(Chothia) or | NO: 108) |
| | | YINPSSGYTEYNQKFKD (SEQ | CDR3: QQYYSYPYT (SEQ ID |
| | | ID NO: 104)(Kabat) | |
| | | CDR3: EGGLLWFDY (SEQ ID | NO: 109) |
| | | NO: 105) | |
| 24. | 16F6.A2- | EVQLVQSGAEVKKPGASVKV | DIVMTQSPDSLAVSLGERAT |
| | CEACAM5- | SCKASGYTFTAYTIHWVRQA | INCKSSHSLLYGNFQNNYLA |
| | B.02-BM- | PGQGLEWMGYINPSSGYTEY | WYQQKPGQPPKLLIYWAST |
| | H1-VH | NQKFKDRTTLTADTSIPTAYM | RESGVPDRFSGSGSGTDFTL |
| | (Humanized)_ | ELSRLRSDDTAVYYCTREGG | TISSLQAEDVAVYYCQQYYS |
| | Variant | LLWFDYWGQGTLVTVSS | YPYTFGGGTKLEIK (SEQ ID |
| | 1(QE) and | (SEQ ID NO: 114) | NO: 119) |
| | Humanized | CDR1: GYTFTAY (SEQ ID | CDR1: |
| | 16F6.A2- | NO: 101)(Chothia) or AYTIH | KSSHSLLYGNFQNNYLA |
| | CEACAM5- | (SEQ ID NO: 102)(Kabat) | (SEQ ID NO: 107) |
| | B.02-BM- | CDR2: NPSSGY (SEQ ID | CDR2: WASTRES (SEQ ID |
| | L1-VL | NO: 103)(Chothia) or | NO: 108) |
| | | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 25. | 16F6.A2- | EVQLVQSGAEVKKPGASVKV | DIVMTQSPDSLAVSLGERAT |
| | CEACAM5- | SCKASGYTFTAYTIHWVRQA | INCKSSHSLLYGNFQNNYLA |
| | B.02-BM- | PGQGLEWMGYINPSSGYTEY | WYQQKPGQPPKLLIYWAST |
| | H1-VH | NQKFKDRTTLTADTSIPTAYM | RESGVPDRFSGSGSGTDFTL |
| | (Humanized)_ | ELSRLRSQDTAVYYCTREGG | TISSLQAEDVAVYYCQQYYS |
| | Variant 2 | LLWFDYWGQGTLVTVSS | YPYTFGGGTKLEIK (SEQ ID |
| | (QE, DQ) | (SEQ ID NO: 115) | NO: 119) |
| | and | CDR1: GYTFTAY (SEQ ID | CDR1: |
| | Humanized | NO: 101)(Chothia) or AYTIH | KSSHSLLYGNFQNNYLA |
| | 16F6.A2- | (SEQ ID NO: 102)(Kabat) | (SEQ ID NO: 107) |
| | CEACAM5- | CDR2: NPSSGY (SEQ ID | CDR2: WASTRES (SEQ ID |
| | B.02-BM- | NO: 103)(Chothia) or | NO: 108) |
| | L1-VL | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 26. | 16F6.A2- | EVQLVQSGAEVKKPGASVK | DIVMTQSPDSLAVSLGERAT |
| | CEACAM5- | MSCKASGYTFTAYTIHWVRQ | INCKSSHSLLYGNFQNNYLA |
| | B.02-BM- | APGQGLEWIGYINPSSGYTEY | WYQQKPGQPPKLLIYWAST |
| | H2-VH | NQKFKDRTTLTADTSIPTAYI | RESGVPDRFSGSGSGTDFTL |
| | (Humanized)_ | ELSRLRSDDTAVYYCTREGG | TISSLQAEDVAVYYCQQYYS |
| | Variant 1 | LLWFDYWGQGTLVTVSS | YPYTFGGGTKLEIK (SEQ ID |
| | (QE) and | (SEQ ID NO: 116) | NO: 119) |
| | Humanized | CDR1: GYTFTAY (SEQ ID | CDR1: |
| | 16F6.A2- | NO: 101)(Chothia) or AYTIH | KSSHSLLYGNFQNNYLA |
| | CEACAM5- | (SEQ ID NO: 102)(Kabat) | (SEQ ID NO: 107) |
| | B.02-BM- | CDR2: NPSSGY (SEQ ID | CDR2: WASTRES (SEQ ID |
| | L1-VL | NO: 103)(Chothia) or | NO: 108) |
| | | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 27. | 16F6.A2- | EVQLVQSGAEVKKPGASVK | DIVMTQSPDSLAVSLGERAT |
| | CEACAM5- | MSCKASGYTFTAYTIHWVRQ | INCKSSHSLLYGNFQNNYLA |
| | B.02-BM- | APGQGLEWIGYINPSSGYTEY | WYQQKPGQPPKLLIYWAST |
| | H2-VH | NQKFKDRTTLTADTSIPTAYI | RESGVPDRFSGSGSGTDFTL |
| | (Humanized)_ | ELSRLRSQDTAVYYCTREGG | TISSLQAEDVAVYYCQQYYS |
| | Variant 2 | LLWFDYWGQGTLVTVSS | YPYTFGGGTKLEIK (SEQ ID |
| | (QHIE, | (SEQ ID NO: 117) | NO: 119) |
| | DH85Q)and | CDR1: GYTFTAY (SEQ ID | CDR1: |
| | Humanized | NO: 101)(Chothia) or AYTIH | KSSHSLLYGNFQNNYLA |
| | 16F6.A2- | (SEQ ID NO: 102)(Kabat) | (SEQ ID NO: 107) |
| | CEACAM5- | CDR2: NPSSGY (SEQ ID | CDR2: WASTRES (SEQ ID |
| | B.02-BM- | NO: 103)(Chothia) or | NO: 108) |
| | L1-VL | YINPSSGYTEYNQKFKD (SEQ | CDR3: QQYYSYPYT (SEQ ID |
| | | ID NO: 104)(Kabat) | NO: 109) |
| | | CDR3: EGGLLWFDY (SEQ ID | |
| | | NO: 105) | |
| 28. | Humanized | X₁VQLVQSGAEVKKPGASVK | DIVMTQSPDSLAVSLGERAT |
| | 16F6.A2- | X₂SCKASGYTFTAYTIHWVRQ | INCKSSHSLLYGNFQNNYLA |
| | CEACAM5- | APGQGLEWX₃GYINPSSGYTE | WYQQKPGQPPKLLIYWAST |
| | B.02-BM | YNQKFKDRTTLTADTSIPTAY | RESGVPDRFSGSGSGTDFTL |
| | and | X₄ELSRLRSX₅DTAVYYCTRE | TISSLQAEDVAVYYCQQYYS |
| | Variants | GGLLWFDYWGQGTLVTVSS | YPYTFGGGTKLEIK (SEQ ID |
| | Consensus | where X₁ is E or Q, X₂ is M or V, | NO: 119) |
| | sequence | X₃ is I or M, X₄ is I or M, X₅ is D | CDR1: |
| | | or Q | KSSHSLLYGNFQNNYLA |
| | | (SEQ ID NO: 118) | (SEQ ID NO: 107) |
| | | CDR1: GYTFTAY (SEQ ID | CDR2: WASTRES (SEQ ID |
| | | NO: 101)(Chothia) or AYTIH | NO: 108) |
| | | (SEQ ID NO: 102)(Kabat) | CDR3: QQYYSYPYT (SEQ ID |
| | | CDR2: NPSSGY (SEQ ID | NO: 109) |
| | | NO: 103)(Chothia) or | |
| | | YINPSSGYTEYNQKFKD (SEQ | |
| | | ID NO: 104)(Kabat) | |
| | | CDR3: EGGLLWFDY (SEQ ID | |
| | | NO: 105) | |

Tier 2

| | | | |
|---|---|---|---|
| 29. | PH_415- | QVQLQESGPGLVKPSGTLSLT | EIVMTQSPSSLSASVGDRVTI |
| | CEACAM5 | CAVSGGSISSSDWWTWVRQP | TCRASQGISNYLAWYQQKP |
| | | PGKGLEWIGEIYHSGSTNYNP | GKVPKLLIYAASTLQSGVPS |
| | | SLKSRVTISVDKSKNQFSLNL | RFSGSGSGTEFTLTISSLQPE |
| | | NSVTAADTAVYYCARGYSGS | DFATYYCQQLNSYPLTFGG |
| | | YFDLDIWGQGTTVTVSS (SEQ | GTKVEIK (SEQ ID NO: 126) |
| | | ID NO: 120) | CDR1: RASQGISNYLA (SEQ |
| | | CDR1: GGSISSSD (SEQ ID | ID NO: 127) |
| | | NO: 121)(Chothia) or SSDWWT | CDR2: AASTLQS (SEQ ID |
| | | (SEQ ID NO: 122)(Kabat) | NO: 128) |
| | | CDR2: YHSGS (SEQ ID | CDR3: QQLNSYPLT (SEQ ID |
| | | NO: 123)(Chothia) or | NO: 129) |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| 30. | PH_416- | QVQLQESGPGLVKPSGTLSLT | EIVMTQSPSSLSASVGDRVTI |
| | CEACAM5 | CAVSGGSISSSDWWTWVRQP | TCRASQGISNYLAWYQQKP |
| | | PGKGLEWIGEIYHSGSTNYNP | GKVPKLLIYAASTLQSGVPS |
| | | SLKSRVTISVDKSKNQFSLNL | RFSGSGSGTEFTLTISSLQPE |
| | | NSVTAADTAVYYCARGYSGS | DFATYYCQQLNSYPLTFGG |
| | | YFDLDIWGQGTTVTVSS (SEQ | GTKVEIK (SEQ ID NO: 126) |
| | | ID NO: 120) | CDR1: RASQGISNYLA (SEQ |
| | | CDR1: GGSISSSD (SEQ ID | ID NO: 127) |
| | | NO: 121)(Chothia) or SSDWWT | CDR2: AASTLQS (SEQ ID |
| | | (SEQ ID NO: 122)(Kabat) | NO: 128) |
| | | CDR2: YHSGS (SEQ ID | CDR3: QQLNSYPLT (SEQ ID |
| | | NO: 123)(Chothia) or | NO: 129) |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| 31. | PH_418- | QVQLQESGPGLVKPSGTLSLT | DIRLTQSPSFLSASVGDRVTI |
| | CEACAM5 | CAVSGGSISSSDWWTWVRQP | TCRASQGISSYLAWYQQKP |
| | | PGKGLEWIGEIYHSGSTNYNP | GKAPKLLIYAASTLQSGVPS |
| | | SLKSRVTISVDKSKNQFSLNL | RFSGSGSGTEFTLTISSLQPE |
| | | NSVTAADTAVYYCARGYSGS | DFATYYCQQLNSYPFTFGPG |
| | | YFDLDIWGQGTTVTVSS (SEQ | TKVDIK (SEQ ID NO: 130) |
| | | ID NO: 120) | CDR1: RASQGISSYLA (SEQ |
| | | CDR1: GGSISSSD (SEQ ID | ID NO: 131) |
| | | NO: 121)(Chothia) or SSDWWT | CDR2: AASTLQS (SEQ ID |
| | | (SEQ ID NO: 122)(Kabat) | NO: 128) |
| | | CDR2: YHSGS (SEQ ID | CDR3: QQLNSYPFT (SEQ ID |
| | | NO: 123)(Chothia) or | NO: 132) |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | PH_418- | | DIRLTQSPSFLSASVGDRVTI |
| | CEACAM5_ | | TCRASQGISSYLAWYQQKP |
| | VL Variant | | GKAPKLLIYAASTLQSGVPS |
| | | | RFSGSGSGTEFTLTISSLQPE |
| | | | DFATYYCQQLNAYPFTFGPG |
| | | | TKVDIK (SEQ ID NO: 133) |
| | | | CDR1: RASQGISSYLA (SEQ |
| | | | ID NO: 131) |
| | | | CDR2: AASTLQS (SEQ ID |
| | | | NO: 128) |
| | | | CDR3: QQLNAYPFT (SEQ ID |
| | | | NO: 134) |
| 32. | PH_419- | QVQLVQSGPGLVKPSGTLSLT | DIVMTQSPSTLSASVGDRVTI |
| | CEACAM5 | CAVSGGSISSSDWWTWVRQP | TCRASQGISSYLAWYQQKP |
| | | PGKGLEWIGEIYHSGSTNYNP | GKAPKLLIYAASTLQSGVPS |
| | | SLKSRVTISVDKSKNQFSLKL | RFSGSGSGTEFTLTISSLQPE |
| | | NSVTAADTAVYYCARGSSGS | DFATYYCQQLNSYPLTFGG |
| | | YFDLDIWGQGTTVTVSS (SEQ | GTKVEIK (SEQ ID NO: 136) |
| | | ID NO: 135) | CDR1: RASQGISSYLA (SEQ |
| | | CDR1: GGSISSSD (SEQ ID | ID NO: 131) |
| | | NO: 121)(Chothia) or SSDWWT | CDR2: AASTLQS (SEQ ID |
| | | (SEQ ID NO: 122)(Kabat) | NO: 128) |
| | | CDR2: YHSGS (SEQ ID | CDR3: QQLNSYPLT (SEQ ID |
| | | NO: 123)(Chothia) or | NO: 129) |
| | | EIYHSGSTNYNPSLKS (SEQ | |
| | | ID NO: 124)(Kabat) | |
| | | CDR3: GSSGSYFDLDI (SEQ ID | |
| | | NO: 337) | |
| | VH | EVQLQESGPGLVKPSGTLSLT | |
| | PH_415- | CAVSGGSISSSDWWTWVRQP | |
| | CEACAM5 | PGKGLEWIGEIYHSGSTNYNP | |
| | (QH1E) | SLKSRVTISVDKSKNQFSLNL | |
| | | NSVTAADTAVYYCARGYSGS | |
| | | YFDLDIWGQGTTVTVSS (SEQ | |
| | | ID NO: 338) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | VH | QVQLQESGPGLVKPSGTLSLT | |
| | PH_415- | CAVSGGSISSSDWWTWVRQP | |
| | CEACAM5 | PGKGLEWIGEIYHSGSTNYNP | |
| | (SH82bA) | SLKSRVTISVDKSKNQFSLNL | |
| | | NAVTAADTAVYYCARGYSG | |
| | | SYFDLDIWGQGTTVTVSS | |
| | | (SEQ ID NO: 339) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | VL_PH_415_ | | EIVMTQSPSSLSASVGDRVTI |
| | CEACAM5 | | TCRASQGISNYLAWYQQKP |
| | (SL93A) | | GKVPKLLIYAASTLQSGVPS |
| | | | RFSGSGSGTEFTLTISSLQPE |
| | | | DFATYYCQQLNAYPLTFGG |
| | | | GTKVEIK (SEQ ID NO: 340) |
| | | | CDR1: RASQGISNYLA (SEQ |
| | | | ID NO: 127) |
| | | | CDR2: AASTLQS (SEQ ID |
| | | | NO: 128) |
| | | | CDR3: QQLNAYPLT (SEQ ID |
| | | | NO: 341) |
| | VH | QVQLQESGPGLVKPSGTLSLT | |
| | PH_416- | CAVSGGSISSSDWWTWVRQP | |
| | CEACAM5 | PGKGLEWIGEIYHSGSTNYNP | |
| | (SH82bA) | SLKSRVTISVDKSKNQFSLNL | |
| | | NAVTAADTAVYYCARGYSG | |
| | | SYFDLDIWGQGTTVTVSS | |
| | | (SEQ ID NO: 339) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | VL | | EIVMTQSPSSLSASVGDRVTI |
| | PH_416- | | TCRASQGISNYLAWYQQKP |
| | CEACAM5 | | GKVPKLLIYAASTLQSGVPS |
| | (SL93A) | | RFSGSGSGTEFTLTISSLQPE |
| | | | DFATYYCQQLNAYPLTFGG |
| | | | GTKVEIK (SEQ ID NO: 340) |
| | | | CDR1: RASQGISNYLA (SEQ |
| | | | ID NO: 127) |
| | | | CDR2: AASTLQS (SEQ ID |
| | | | NO: 128) |
| | | | CDR3: QQLNAYPLT (SEQ ID |
| | | | NO: 341) |
| | VH PH_418- | QVQLQESGPGLVKPSGTLSLT | |
| | CEACAM5 | CAVSGGSISSSDWWTWVRQP | |
| | (SH82bA) | PGKGLEWIGEIYHSGSTNYNP | |
| | | SLKSRVTISVDKSKNQFSLNL | |
| | | NAVTAADTAVYYCARGYSG | |
| | | SYFDLDIWGQGTTVTVSS | |
| | | (SEQ ID NO: 339) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | VL | | DIRLTQSPSFLSASVGDRVTI |
| | PH_418- | | TCRASQGISSYLAWYQQKP |
| | CEACAM5 | | GKAPKLLIYAASTLQSGVPS |
| | (SL93A) | | RFSGSGSGTEFTLTISSLQPE |
| | | | DFATYYCQQLNAYPFTFGPG |
| | | | TKVDIK (SEQ ID NO: 133) |
| | | | CDR1: RASQGISSYLA (SEQ |
| | | | ID NO: 131) |
| | | | CDR2: AASTLQS (SEQ ID |
| | | | NO: 128) |
| | | | CDR3: QQLNAYPFT (SEQ ID |
| | | | NO: 134) |
| | VH_PH_419- | EVQLVQSGPGLVKPSGTLSLT | |
| | CEACAM5 | CAVSGGSISSSDWWTWVRQP | |
| | (QH1E) | PGKGLEWIGEIYHSGSTNYNP | |
| | | SLKSRVTISVDKSKNQFSLKL | |
| | | NSVTAADTAVYYCARGSSGS | |
| | | YFDLDIWGQGTTVTVSS (SEQ | |
| | | ID NO: 342) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GSSGSYFDLDI (SEQ ID | |
| | | NO: 337) | |
| | VH_PH_419- | QVQLVQSGPGLVKPSGTLSLT | |
| | CEACAM5 | CAVSGGSISSSDWWTWVRQP | |
| | (SH82bA) | PGKGLEWIGEIYHSGSTNYNP | |
| | | SLKSRVTISVDKSKNQFSLKL | |
| | | NAVTAADTAVYYCARGSSGS | |
| | | YFDLDIWGQGTTVTVSS (SEQ | |
| | | ID NO: 343) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GSSGSYFDLDI (SEQ ID | |
| | | NO: 337) | |
| | VL | | DIVMTQSPSTLSASVGDRVTI |
| | PH_419- | | TCRASQGISSYLAWYQQKP |
| | CEACAM5 | | GKAPKLLIYAASTLQSGVPS |
| | (SL93A) | | RFSGSGSGTEFTLTISSLQPE |
| | | | DFATYYCQQLNAYPLTFGG |
| | | | GTKVEIK (SEQ ID NO: 344) |
| | | | CDR1: RASQGISSYLA (SEQ |
| | | | ID NO: 131) |
| | | | CDR2: AASTLQS (SEQ ID |
| | | | NO: 128) |
| | | | CDR3: QQLNAYPLT (SEQ ID |
| | | | NO: 341) |
| 33. | VH | X₁VQLX₂X₃SGPGLVKPSGTLS | X₁IVMTQSPSX₂LSASVGDRV |
| | Consensus | LTCAVSGGSISSSDWWTWVR | TITCRASQGIS |
| | Sequences | QPPGKGLEWIGEIYHSGSTNY | X₃YLAWYQQKPGKX₄PKLLI |
| | [PH_415- | NPSLKSRVTISVDKSKNQFSL | YAASTLQSGVPSRFSGSGSG |
| | CEACAM5; | X₄LNX₅VTAADTAVYYCARG | TEFTLTISSLQPEDFATYYCQ |
| | PH_416- | X₆SGSYFDLDIWGQGTTVTVS | QLNX₅YPLTFGGGTKVEIK |
| | CEACAM5; | S | where X₁ is E or D, X₂ is T or S, |
| | PH_419- | where X₁ is E or Q, X₂ is Q or V, | X₃ is N or S, X₄ is V or A, and |
| | CEACAM5; | X₃ is E or Q, X₄ is K or N, X₅ is S | X₅ is S or A |
| | PH_418- | or A, and X₆ is S or Y | (SEQ ID NO: 139) |
| | CEACAM5] | (SEQ ID NO: 137) | CDR1: RASQGISXYLA where |
| | and | CDR1: GGSISSSD (SEQ ID | X is N or S (SEQ ID NO: 140) |
| | VL | NO: 121)(Chothia) or SSDWWT | CDR2: AASTLQS (SEQ ID |
| | Consensus | (SEQ ID NO: 122)(Kabat) | NO: 128) |
| | Sequences | CDR2: YHSGS (SEQ ID | CDR3: QQLNXYPLT where X |
| | [PH_415- | NO: 123)(Chothia) or | is S or A (SEQ ID NO: 141) |
| | CEACAM5 | EIYHSGSTNYNPSLKS (SEQ | |
| | PH_416- | ID NO: 124)(Kabat) | |
| | CEACAM5 | CDR3: GXSGSYFDLDI where X | |
| | PH_419- | is S or Y (SEQ ID NO: 138) | |
| | CEACAM5] | | |
| 34. | VH | X₁VQLX₂X₃SGPGLVKPSGTLS | DIRLTQSPSFLSASVGDRVTI |
| | Consensus | LTCAVSGGSISSSDWWTWVR | TCRASQGISSYLAWYQQKP |
| | Sequences | QPPGKGLEWIGEIYHSGSTNY | GKAPKLLIYAASTLQSGVPS |
| | [PH_415- | NPSLKSRVTISVDKSKNQFSL | RFSGSGSGTEFTLTISSLQPE |
| | CEACAM5; | X₄LNX₅VTAADTAVYYCARG | DFATYYCQQLNXYPFTFGPG |
| | PH_416- | X₆SGSYFDLDIWGQGTTVTVS | TKVDIK |
| | CEACAM5; | S | where X is S or A |
| | PH_419- | where X₁ is Q or E, X₂ is Q or V, | (SEQ ID NO: 142) |
| | CEACAM5; | X₃ is E or Q, X₄ is K or N, X₅ is S | CDR1: RASQGISSYLA (SEQ |
| | PH_418- | or A, and X₆ is S or Y | ID NO: 131) |
| | CEACAM5] | (SEQ ID NO: 137) | CDR2: AASTLQS (SEQ ID |
| | and | CDR1: GGSISSSD (SEQ ID | NO: 128) |
| | VL | NO: 121)(Chothia) or SSDWWT | CDR3: QQLNXYPFT where X |
| | Consensus | (SEQ ID NO: 122)(Kabat) | is S or A (SEQ ID NO: 143) |
| | Sequence | CDR2: YHSGS (SEQ ID | |
| | [PH_418- | NO: 123)(Chothia) or | |
| | CEACAM5] | EIYHSGSTNYNPSLKS (SEQ | |
| | | ID NO: 124)(Kabat) | |
| | | CDR3: GXSGSYFDLDI where X | |
| | | is S or Y (SEQ ID NO: 138) | |
| 35. | PH_417- | QVQLQESGPGLVKPSGTLSLT | DIQVTQSPATLSVSPGERVTL |
| | CEACAM5 | CAVSGGSISSSKWWSWVRQS | SCRASRSVRSNLAWYQQKP |
| | | PGKGLEWIGEIFHSGSINHNTS | GQAPRLLIYGASSRATGIPDR |
| | | FKSRVTISVDKSKNQFSLKLS | FSGSGSGTDFTLTISSLQPDD |
| | | SVTAADTAVYYCARGGSGSY | FAVYYCQQYNNWPITFGQG |
| | | DAFDIWGQGTMVTVSS (SEQ | TRLEIK (SEQ ID NO: 150) |
| | | ID NO: 144) | CDR1: RASRSVRSNLA (SEQ |
| | | CDR1: GGSISSSK (SEQ ID | ID NO: 151) |
| | | NO: 145)(Chothia) or SSKWWS | CDR2: GASSRAT (SEQ ID |
| | | (SEQ ID NO: 146)(Kabat) | NO: 41) |
| | | CDR2: FHSGS (SEQ ID | CDR3: QQYNNWPIT (SEQ ID |
| | | NO: 147)(Chothia) or | NO: 152) |
| | | EIFHSGSINHNTSFKS (SEQ ID | |
| | | NO: 148)(Kabat) | |
| | | CDR3: GGSGSYDAFDI (SEQ | |
| | | ID NO: 149) | |
| | VH PH_417- | EVQLQESGPGLVKPSGTLSLT | |
| | CEACAM5 | CAVSGGSISSSKWWSWVRQS | |
| | (QH1E) | PGKGLEWIGEIFHSGSINHNTS | |
| | | FKSRVTISVDKSKNQFSLKLS | |
| | | SVTAADTAVYYCARGGSGSY | |
| | | DAFDIWGQGTMVTVSS (SEQ | |
| | | ID NO: 345) | |
| | | CDR1: GGSISSSK (SEQ ID | |
| | | NO: 145)(Chothia) or SSKWWS | |
| | | (SEQ ID NO: 146)(Kabat) | |
| | | CDR2: FHSGS (SEQ ID | |
| | | NO: 147)(Chothia) or | |
| | | EIFHSGSINHNTSFKS (SEQ ID | |
| | | NO: 148)(Kabat) | |
| | | CDR3: GGSGSYDAFDI (SEQ | |
| | | ID NO: 149) | |
| | VH PH_417- | QVQLQESGPGLVKPSGTLSLT | |
| | CEACAM5 | CAVSGGSISSSKWWSWVRQS | |
| | (NH60Q) | PGKGLEWIGEIFHSGSINHQTS | |
| | | FKSRVTISVDKSKNQFSLKLS | |
| | | SVTAADTAVYYCARGGSGSY | |
| | | DAFDIWGQGTMVTVSS (SEQ | |
| | | ID NO: 346) | |
| | | CDR1: GGSISSSK (SEQ ID | |
| | | NO: 145)(Chothia) or SSKWWS | |
| | | (SEQ ID NO: 146)(Kabat) | |
| | | CDR2: FHSGS (SEQ ID | |
| | | NO: 147)(Chothia) or | |
| | | EIFHSGSINHQTSFKS (SEQ ID | |
| | | NO: 347)(Kabat) | |
| | | CDR3: GGSGSYDAFDI (SEQ | |
| | | ID NO: 149) | |
| | VH PH_417- | QVQLQESGPGLVKPSGTLSLT | |
| | CEACAM5 | CAVSGGSISSSKWWSWVRQS | |
| | (SH62A) | PGKGLEWIGEIFHSGSINHNT | |
| | | AFKSRVTISVDKSKNQFSLKL | |
| | | SSVTAADTAVYYCARGGSGS | |
| | | YDAFDIWGQGTMVTVSS | |
| | | (SEQ ID NO: 348) | |
| | | CDR1: GGSISSSK (SEQ ID | |
| | | NO: 145)(Chothia) or SSKWWS | |
| | | (SEQ ID NO: 146)(Kabat) | |
| | | CDR2: FHSGS (SEQ ID | |
| | | NO: 147)(Chothia) or | |
| | | EIFHSGSINHNTAFKS (SEQ ID | |
| | | NO: 349)(Kabat) | |
| | | CDR3: GGSGSYDAFDI (SEQ | |
| | | ID NO: 149) | |
| 36. | PH_417- | X₁VQLQESGPGLVKPSGTLSL | DIQVTQSPATLSVSPGERVTL |
| | CEACAM5 | TCAVSGGSISSSKWWSWVRQ | SCRASRSVRSNLAWYQQKP |
| | Consensus | SPGKGLEWIGEIFHSGSINHX₂ | GQAPRLLIYGASSRATGIPDR |
| | Sequence | TX₃FKSRVTISVDKSKNQFSLK | FSGSGSGTDFTLTISSLQPDD |
| | | LSSVTAADTAVYYCARGGSG | FAVYYCQQYNNWPITFGQG |
| | | SYDAFDIWGQGTMVTVSS | TRLEIK (SEQ ID NO: 150) |
| | | where X₁ is Q or E, X₂ is N or Q, | CDR1: RASRSVRSNLA (SEQ |
| | | and X₃ is S or A | ID NO: 151) |
| | | (SEQ ID NO: 153) | CDR2: GASSRAT (SEQ ID |
| | | CDR1: GGSISSSK (SEQ ID | NO: 41) |
| | | NO: 145)(Chothia) or SSKWWS | CDR3: QQYNNWPIT (SEQ ID |
| | | (SEQ ID NO: 146)(Kabat) | NO: 152) |
| | | CDR2: FHSGS (SEQ ID | |
| | | NO: 147)(Chothia) or | |
| | | EIFHSGSINHX₁TX₂FKS (Kabat) | |
| | | where X₁ is N or Q and X₂ is S or | |
| | | A (SEQ ID NO: 154) | |
| | | CDR3: GGSGSYDAFDI (SEQ | |
| | | ID NO: 149) | |
| 37. | PH_421- | QVQLQESGPGLVKPSGTLSLT | EIVLTQSPSFLSASVGDRVTI |
| | CEACAM5 | CAVSGGSISSSDWWTWVRQP | TCRASQGISSYLAWYQQKP |
| | | PGKGLEWIGEIYHSGSTNYNP | GKAPKLLIYAASTLQSGVPS |
| | | SLKSRVTISVDKSKNQFSLNL | RFSGSGSGTEFTLTISSLQPE |
| | | NSVTAADTAVYYCARGYSGS | DFATYYCQQLNSYPFTFGPG |
| | | YFDLDIWGQGTTVTVSSGS | TKLEIK (SEQ ID NO: 156) |
| | | (SEQ ID NO: 155) | CDR1: RASQGISSYLA (SEQ |
| | | CDR1: GGSISSSD (SEQ ID | ID NO: 131) |
| | | NO: 121)(Chothia) or SSDWWT | CDR2: AASTLQS (SEQ ID |
| | | (SEQ ID NO: 122)(Kabat) | NO: 128) |
| | | CDR2: YHSGS (SEQ ID | CDR3: QQLNSYPFT (SEQ ID |
| | | NO: 123)(Chothia) or | NO: 132) |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | PH_421- | | EIVLTQSPSFLSASVGDRVTI |
| | CEACAM5_ | | TCRASQGISSYLAWYQQKP |
| | VL Variant | | GKAPKLLIYAASTLQSGVPS |
| | | | RFSGSGSGTEFTLTISSLQPE |
| | | | DFATYYCQQLNAYPFTFGPG |
| | | | TKLEIK (SEQ ID NO: 157) |
| | | | CDR1: RASQGISSYLA (SEQ |
| | | | ID NO: 131) |
| | | | CDR2: AASTLQS (SEQ ID |
| | | | NO: 128) |
| | | | CDR3: QQLNAYPFT (SEQ ID |
| | | | NO: 134) |
| | VH PH_421- | EVQLQESGPGLVKPSGTLSLT | |
| | CEACAM5 | CAVSGGSISSSDWWTWVRQP | |
| | (QH1E) | PGKGLEWIGEIYHSGSTNYNP | |
| | | SLKSRVTISVDKSKNQFSLNL | |
| | | NSVTAADTAVYYCARGYSGS | |
| | | YFDLDIWGQGTTVTVSSGS | |
| | | (SEQ ID NO: 350) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| | VH PH_421- | QVQLQESGPGLVKPSGTLSLT | |
| | CEACAM5 | CAVSGGSISSSDWWTWVRQP | |
| | (SH82bA) | PGKGLEWIGEIYHSGSTNYNP | |
| | | SLKSRVTISVDKSKNQFSLNL | |
| | | NAVTAADTAVYYCARGYSG | |
| | | SYFDLDIWGQGTTVTVSSGS | |
| | | (SEQ ID NO: 351) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| 38. | PH_421- | X₁VQLQESGPGLVKPSGTLSL | EIVLTQSPSFLSASVGDRVTI |
| | CEACAM5 | TCAVSGGSISSSDWWTWVRQ | TCRASQGISSYLAWYQQKP |
| | Consensus | PPGKGLEWIGEIYHSGSTNYN | GKAPKLLIYAASTLQSGVPS |
| | Sequence | PSLKSRVTISVDKSKNQFSLN | RFSGSGSGTEFTLTISSLQPE |
| | | LNX₂VTAADTAVYYCARGYS | DFATYYCQQLNXYPFTFGPG |
| | | GSYFDLDIWGQGTTVTVSSGS | TKLEIK |
| | | where X₁ is Q or E and X₂ is S or | where X is S or A |
| | | A | (SEQ ID NO: 159) |
| | | (SEQ ID NO: 158) | CDR1: RASQGISSYLA (SEQ |
| | | CDR1: GGSISSSD (SEQ ID | ID NO: 131) |
| | | NO: 121)(Chothia) or SSDWWT | CDR2: AASTLQS (SEQ ID |
| | | (SEQ ID NO: 122)(Kabat) | NO: 128) |
| | | CDR2: YHSGS (SEQ ID | CDR3: QQLNXYPFT where X |
| | | NO: 123)(Chothia) or | is S or A (SEQ ID NO: 143) |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| 39. | 1078_G03- | QVQLVESGGGLVQPGGSLRL | DIRMTQSPSTLSASVGDRVTI |
| | CEACAM5 | SCAASGFTFSSYAMSWVRQA | TCRASQSISSWLAWYQQKP |
| | | PGKGLEWVSEISGSGDRTSYA | GKAPKLLIYKASSLESGVPS |
| | | DSVKGRFTISRDNSKNRLYLQ | RFSGSGSGTEFTLTISSLQPD |
| | | MNRLRTEDTAVYYCAKDLGP | DFATYYCQQYNSYSTWTFG |
| | | SGWYGLFDYWGQGTLVTVS | QGTKVDIK (SEQ ID NO: 164) |
| | | S (SEQ ID NO: 160) | CDR1: RASQSISSWLA (SEQ |
| | | CDR1: GFTFSSY (SEQ ID | ID NO: 165) |
| | | NO: 2)(Chothia) or SYAMS | CDR2: KASSLES (SEQ ID |
| | | (SEQ ID NO: 3)(Kabat) | NO: 8) |
| | | CDR2: SGSGDR (SEQ ID | CDR3: QQYNSYSTWT (SEQ |
| | | NO: 161)(Chothia) or | ID NO: 166) |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| | 1078_G03- | | DIRMTQSPSTLSASVGDRVTI |
| | CEACAM5_ | | TCRASQSISSWLAWYQQKP |
| | VL Variant | | GKAPKLLIYKASSLESGVPS |
| | | | RFSGSGSGTEFTLTISSLQPD |
| | | | DFATYYCQQYNA |
| | | | YSTWTFGQGTKVDIK (SEQ |
| | | | ID NO: 167) |
| | | | CDR1: RASQSISSWLA (SEQ |
| | | | ID NO: 165) |
| | | | CDR2: KASSLES (SEQ ID |
| | | | NO: 8) |
| | | | CDR3: QQYNAYSTWT (SEQ |
| | | | ID NO: 168) |
| | VH_1078_ | EVQLVESGGGLVQPGGSLRL | |
| | G03- | SCAASGFTFSSYAMSWVRQA | |
| | CEACAM5 | PGKGLEWVSEISGSGDRTSYA | |
| | (QH1E) | DSVKGRFTISRDNSKNRLYLQ | |
| | | MNRLRTEDTAVYYCAKDLGP | |
| | | SGWYGLFDYWGQGTLVTVS | |
| | | S (SEQ ID NO: 352) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| | VH | QVQLVESGGGLVQPGGSLRL | |
| | 1078_G03- | SCAASGFTFSSYAMSWVRQA | |
| | CEACAM5 | PGKGLEWVSEISGSGDRTSYA | |
| | (SH62A) | DAVKGRFTISRDNSKNRLYL | |
| | | QMNRLRTEDTAVYYCAKDL | |
| | | GPSGWYGLFDYWGQGTLVT | |
| | | VSS (SEQ ID NO: 353) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADAVKG (SEQ | |
| | | ID NO: 720)(Kabat) | |
| | | CDR3: DLGPSGWYGLFDY | |
| | | (SEQ ID NO: 163) | |
| | VH | QVQLVESGGGLVQPGGSLRL | |
| | 1078_G03CEACAM5 | SCAASGFTFSSYAMSWVRQA | |
| | (NH73A) | PGKGLEWVSEISGSGDRTSYA | |
| | | DSVKGRFTISRDASKNRLYLQ | |
| | | MNRLRTEDTAVYYCAKDLGP | |
| | | SGWYGLFDYWGQGTLVTVS | |
| | | S (SEQ ID NO: 354) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADAVKG (SEQ | |
| | | ID NO: 720)(Kabat) | |
| | | CDR3: DLGPSGWYGLFDY | |
| | | (SEQ ID NO: 163) | |
| | VH | QVQLVESGGGLVQPGGSLRL | |
| | 1078_G03- | SCAASGFTFSSYAMSWVRQA | |
| | CEACAM5 | PGKGLEWVSEISGSGDRTSYA | |
| | (SH74A) | DSVKGRFTISRDNAKNRLYL | |
| | | QMNRLRTEDTAVYYCAKDL | |
| | | GPSGWYGLFDYWGQGTLVT | |
| | | VSS (SEQ ID NO: 355) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADAVKG (SEQ | |
| | | ID NO: 720)(Kabat) | |
| | | CDR3: DLGPSGWYGLFDY | |
| | | (SEQ ID NO: 163) | |
| 40. | 1078_G03- | X₁VQLVESGGGLVQPGGSLRL | DIRMTQSPSTLSASVGDRVTI |
| | CEACAM5 | SCAASGFTFSSYAMSWVRQA | TCRASQSISSWLAWYQQKP |
| | Consensus | PGKGLEWVSEISGSGDRTSYA | GKAPKLLIYKASSLESGVPS |
| | Sequence | DX₂VKGRFTISRDX₃X₄KNRLY | RFSGSGSGTEFTLTISSLQPD |
| | | LQMNRLRTEDTAVYYCAKD | DFATYYCQQYNX |
| | | LGPSGWYGLFDYWGQGTLV | YSTWTFGQGTKVDIK |
| | | TVSS | where X is S or A |
| | | where X₁ is Q or E, and X₂ is S or | (SEQ ID NO: 171) |
| | | A, X₃ is N or A, and X₄ is S or A | CDR1: RASQSISSWLA (SEQ |
| | | (SEQ ID NO: 169) | ID NO: 165) |
| | | CDR1: GFTFSSY (SEQ ID | CDR2: KASSLES (SEQ ID |
| | | NO: 2)(Chothia) or SYAMS | NO: 8) |
| | | (SEQ ID NO: 3)(Kabat) | CDR3: QQYNXYSTWT where |
| | | CDR2: SGSGDR (SEQ ID | X is S or A (SEQ ID NO: 172) |
| | | NO: 161)(Chothia) or | |
| | | EISGSGDRTSYADXVKG | |
| | | (Kabat) where X is S or A (SEQ | |
| | | ID NO: 170) | |
| | | CDR3: DLGPSGWYGLFDY | |
| | | (SEQ ID NO: 163) | |
| 41. | Murine | EVQLQESGAELVRSGASVKL | QIVLSQSPAIMSASPGEKVTI |
| | 1A1.A3- | SCTASDFNIKDSYMHWVTQR | TCSTSSSVSYMHWFQQKPG |
| | CEACAM5- | PEQGLEWIGWIDPENGDTEY | TSPKLWIYSTSNLASGVPAR |
| | B.02 | APKFQGKATMTADTSSNTAY | FSGSGSGTSYSLTISRMEAED |
| | | LHLSSLTSEDTAVYYCNVITT | AATYYCQQRSNYPLTFGAG |
| | | VVNYAMDYWGQGTSVTVSS | TKLELK (SEQ ID NO: 179) |
| | | (SEQ ID NO: 173) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPENGD (SEQ ID | CDR3: QQRSNYPLT (SEQ ID |
| | | NO: 176)(Chothia) or | NO: 182) |
| | | WIDPENGDTEYAPKFQG | |
| | | (SEQ ID NO: 177)(Kabat) | |
| | | CDR3: ITTVVNYAMDY (SEQ | |
| | | ID NO: 178) | |
| 42. | Humanized | EVQLVQSGAEVVKPGATVKL | EIVLTQSPATLSASPGERATL |
| | 1A1.A3- | SCKASDFNIKDSYMHWVQQA | SCSTSSSVSYMHWFQQKPG |
| | CEACAM5- | PGKGLEWIGWIDPENGDTEY | QAPRLWIYSTSNLASGVPAR |
| | B.02-BM- | APKFQGRATITADTSTDTAYL | FSGSGSGTDYTLTISSLEPED |
| | H1_NG | ELSSLRSEDTAVYYCNVITTV | FAVYYCQQRSNYPLTFGAG |
| | | VNYAMDYWGQGTLVTVSS | TKVEIK (SEQ ID NO: 184) |
| | | (SEQ ID NO: 183) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPENGD (SEQ ID | CDR3: QQRSNYPLT (SEQ ID |
| | | NO: 176)(Chothia) or | NO: 182) |
| | | WIDPENGDTEYAPKFQG | |
| | | (SEQ ID NO: 177)(Kabat) | |
| | | CDR3: ITTVVNYAMDY (SEQ | |
| | | ID NO: 178) | |
| 43. | Humanized | EVQLVQSGAEVVKPGATVKL | EIVLTQSPATLSASPGERATL |
| | 1A1.A3- | SCKASDFNIKDSYMHWVQQA | SCSTSSSVSYMHWFQQKPG |
| | CEACAM5- | PGKGLEWIGWIDPESGDTEY | QAPRLWIYSTSNLASGVPAR |
| | B.02-BM- | APKFQGRATITADTSTDTAYL | FSGSGSGTDYTLTISSLEPED |
| | H1_SG | ELSSLRSEDTAVYYCNVITTV | FAVYYCQQRSNYPLTFGAG |
| | | VNYAMDYWGQGTLVTVSS | TKVEIK (SEQ ID NO: 184) |
| | | (SEQ ID NO: 185) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPESGD (SEQ ID | CDR3: QQRSNYPLT (SEQ ID |
| | | NO: 186)(Chothia) or | NO: 182) |
| | | WIDPESGDTEYAPKFQG (SEQ | |
| | | ID NO: 187)(Kabat) | |
| | | CDR3: ITTVVNYAMDY (SEQ | |
| | | ID NO: 178) | |
| 44. | Humanized | EVQLQQSGAEVVKPGATVKL | EIVLTQSPATLSASPGERATL |
| | 1A1.A3- | SCKASDFNIKDSYMHWVTQA | SCSTSSSVSYMHWFQQKPG |
| | CEACAM5- | PGKGLEWIGWIDPENGDTEY | QAPRLWIYSTSNLASGVPAR |
| | B.02-BM- | APKFQGRATMTADTSTDTAY | FSGSGSGTDYTLTISSLEPED |
| | H2_NG | LELSSLRSEDTAVYYCNVITT | FAVYYCQQRSNYPLTFGAG |
| | | VVNYAMDYWGQGTLVTVSS | TKVEIK (SEQ ID NO: 184) |
| | | (SEQ ID NO: 188) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPENGD (SEQ ID | CDR3: QQRSNYPLT (SEQ ID |
| | | NO: 176)(Chothia) or | NO: 182) |
| | | WIDPENGDTEYAPKFQG | |
| | | (SEQ ID NO: 177)(Kabat) | |
| | | CDR3: ITTVVNYAMDY (SEQ | |
| | | ID NO: 178) | |
| 45. | Humanized | EVQLQQSGAEVVKPGATVKL | EIVLTQSPATLSASPGERATL |
| | 1A1.A3- | SCKASDFNIKDSYMHWVTQA | SCSTSSSVSYMHWFQQKPG |
| | CEACAM5- | PGKGLEWIGWIDPESGDTEY | QAPRLWIYSTSNLASGVPAR |
| | B.02-BM- | APKFQGRATMTADTSTDTAY | FSGSGSGTDYTLTISSLEPED |
| | H2_SG | LELSSLRSEDTAVYYCNVITT | FAVYYCQQRSNYPLTFGAG |
| | | VVNYAMDYWGQGTLVTVSS | TKVEIK (SEQ ID NO: 184) |
| | | (SEQ ID NO: 189) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPESGD (SEQ ID | CDR3: QQRSNYPLT (SEQ ID |
| | | NO: 186)(Chothia) or | NO: 182) |
| | | WIDPESGDTEY APKFQG (SEQ | |
| | | ID NO: 187)(Kabat) | |
| | | CDR3: ITTVVNYAMDY (SEQ | |
| | | ID NO: 178) | |
| 46. | Humanized | EVQLQQSGAEVVKPGATVKL | EIVLTQSPATLSASPGERATL |
| | 1A1.A3- | SCKASDFNIKDSYMHWVTQA | SCSTSSSVSYMHWFQQKPG |
| | CEACAM5- | PGKGLEWIGWIDPEQGDTEY | QAPRLWIYSTSNLASGVPAR |
| | B.02-BM- | APKFQGRATMTADTSTDTAY | FSGSGSGTDYTLTISSLEPED |
| | H2_QG | LELSSLRSEDTAVYYCNVITT | FAVYYCQQRSNYPLTFGAG |
| | | VVNYAMDYWGQGTLVTVSS | TKVEIK (SEQ ID NO: 184) |
| | | (SEQ ID NO: 190) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPEQGD (SEQ ID | CDR3: QQRSNYPLT (SEQ ID |
| | | NO: 191)(Chothia) or | NO: 182) |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| | VH_ | EVQLVQSGAEVVKPGATVKL | |
| | Humanized | SCKASDFNIKDAYMHWVQQ | |
| | 1A1.A3- | APGKGLEWIGWIDPENGDTE | |
| | CEACAM5- | YAPKFQGRATITADTSTDTAY | |
| | B.02-BM- | LELSSLRSEDTAVYYCNVITT | |
| | H1_(SH32A) | VVNYAMDYWGQGTLVTVSS | |
| | | (SEQ ID NO: 356) | |
| | | CDR1: DFNIKDA (SEQ ID | |
| | | NO: 200)(Chothia) or DAYMH | |
| | | (SEQ ID NO: 201)(Kabat) | |
| | | CDR2: DPENGD (SEQ ID | |
| | | NO: 176)(Chothia) or | |
| | | WIDPENGDTEYAPKFQG | |
| | | (SEQ ID NO: 177)(Kabat) | |
| | | CDR3: ITTVVNYAMDY (SEQ | |
| | | ID NO: 178) | |
| | VH | EVQLVQSGAEVVKPGATVKL | |
| | Humanized | SCKASDFNIKDSYMHWVQQA | |
| | 1A1.A3- | PGKGLEWIGWIDPEQGDTEY | |
| | CEACAM5- | APKFQGRATITADTSTDTAYL | |
| | B.02-BM- | ELSSLRSEDTAVYYCNVITTV | |
| | H1_(NH54Q) | VNYAMDYWGQGTLVTVSS | |
| | | (SEQ ID NO: 198) | |
| | | CDR1: DFNIKDS (SEQ ID | |
| | | NO: 174)(Chothia) or DSYMH | |
| | | (SEQ ID NO: 175)(Kabat) | |
| | | CDR2: DPEQGD (SEQ ID | |
| | | NO: 191)(Chothia) or | |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| | VH | EVQLVQSGAEVVKPGATVKL | |
| | Humanized | SCKASDFNIKDSYMHWVQQA | |
| | 1A1.A3- | PGKGLEWIGWIDPESGDTEY | |
| | CEACAM5- | APKFQGRATITADTSTDTAYL | |
| | B.02-BM- | ELSSLRSEDTAVYYCNVITTV | |
| | H1_(NH54S) | VNYAMDYWGQGTLVTVSS | |
| | | (SEQ ID NO: 185) | |
| | | CDR1: DFNIKDS (SEQ ID | |
| | | NO: 174)(Chothia) or DSYMH | |
| | | (SEQ ID NO: 175)(Kabat) | |
| | | CDR2: DPESGD (SEQ ID | |
| | | NO: 186)(Chothia) or | |
| | | WIDPESGDTEYAPKFQG (SEQ | |
| | | ID NO: 187)(Kabat) | |
| | | CDR3: ITTVVNYAMDY (SEQ | |
| | | ID NO: 178) | |
| | Humanized | EVQLQQSGAEVVKPGATVKL | |
| | 1A1.A3- | SCKASDFNIKDSYMHWVTQA | |
| | CEACAM5- | PGKGLEWIGWIDPEQGDTEY | |
| | B.02-BM- | APKFQGRATMTADTSTDTAY | |
| | H2_(NH54Q) | LELSSLRSEDTAVYYCNVITT | |
| | | VVNYAMDYWGQGTLVTVSS | |
| | | (SEQ ID NO: 190) | |
| | | CDR1: DFNIKDS (SEQ ID | |
| | | NO: 174)(Chothia) or DSYMH | |
| | | (SEQ ID NO: 175)(Kabat) | |
| | | CDR2: DPEQGD (SEQ ID | |
| | | NO: 191)(Chothia) or | |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| | Humanized | EVQLQQSGAEVVKPGATVKL | |
| | 1A1.A3- | SCKASDFNIKDSYMHWVTQA | |
| | CEACAM5- | PGKGLEWIGWIDPESGDTEY | |
| | B.02-BM- | APKFQGRATMTADTSTDTAY | |
| | H2_(NH54S) | LELSSLRSEDTAVYYCNVITT | |
| | | VVNYAMDYWGQGTLVTVSS | |
| | | (SEQ ID NO: 189) | |
| | | CDR1: DFNIKDS (SEQ ID | |
| | | NO: 174)(Chothia) or DSYMH | |
| | | (SEQ ID NO: 175)(Kabat) | |
| | | CDR2: DPESGD (SEQ ID | |
| | | NO: 186)(Chothia) or | |
| | | WIDPESGDTEY APKFQG (SEQ | |
| | | ID NO: 187)(Kabat) | |
| | | CDR3: ITTVVNYAMDY (SEQ | |
| | | ID NO: 178) | |
| 47. | Humanized | EVQLX₁QSGAEVVKPGATVK | EIVLTQSPATLSASPGERATL |
| | 1A1.A3- | LSCKASDFNIKDX₂YMHWVX₃ | SCSTSSSVSYMHWFQQKPG |
| | CEACAM5- | QAPGKGLEWIGWIDPEX₄GDT | QAPRLWIYSTSNLASGVPAR |
| | B.02-BM- | EYAPKFQGRATX₅TADTSTDT | FSGSGSGTDYTLTISSLEPED |
| | H1_NG- | AYLELSSLRSEDTAVYYCNVI | FAVYYCQQRSNYPLTFGAG |
| | VH; | TTVVNYAMDYWGQGTLVTV | TKVEIK (SEQ ID NO: 184) |
| | 1A1.A3- | SS | CDR1: STSSSVSYMH (SEQ |
| | CEACAM5- | where X₁ is Q or V, X₂ is S or A, | ID NO: 180) |
| | B.02-BM- | X₃ is T or Q, X₄ is Q, S or N, and | CDR2: STSNLAS (SEQ ID |
| | H1_SG-VH; | X₅ is M or I | NO: 181) |
| | 1A1.A3- | (SEQ ID NO: 193) | CDR3: QQRSNYPLT (SEQ ID |
| | CEACAM5- | CDR1: DFNIKDX (SEQ ID | NO: 182) |
| | B.02-BM- | NO: 194)(Chothia) or DXYMH | |
| | H2_NG- | (Kabat) where X is S or A (SEQ | |
| | VH; | ID NO: 195) | |
| | 1A1.A3- | CDR2: DPEXGD (SEQ ID | |
| | CEACAM5- | NO: 196)(Chothia) or | |
| | B.02-BM- | WIDPEXGDTEYAPKFQG | |
| | H2_SG-VH; | (Kabat) where X is Q, S or N | |
| | 1A1.A3- | (SEQ ID NO: 197) | |
| | CEACAM5- | CDR3: ITTVVNYAMDY (SEQ | |
| | B.02-BM- | ID NO: 178) | |
| | H2_QG-VH | | |
| | Consensus | | |
| | Sequence | | |
| 48. | Humanized | EVQLVQSGAEVVKPGATVKL | EIVLTQSPATLSASPGERATL |
| | 1A1.A3- | SCKASDFNIKDSYMHWVQQA | SCSTSSSVSYMHWFQQKPG |
| | CEACAM5- | PGKGLEWIGWIDPEQGDTEY | QAPRLWIYSTSNLASGVPAR |
| | B.02-BM- | APKFQGRATITADTSTDTAYL | FSGSGSGTDYTLTISSLEPED |
| | H1_QG-VH | ELSSLRSEDTAVYYCNVITTV | FAVYYCQQRSNYPLTFGAG |
| | | VNYAMDYWGQGTLVTVSS | TKVEIK (SEQ ID NO: 184) |
| | | (SEQ ID NO: 198) | CDR1: STSSSVSYMH (SEQ |
| | | CDR1: DFNIKDS (SEQ ID | ID NO: 180) |
| | | NO: 174)(Chothia) or DSYMH | CDR2: STSNLAS (SEQ ID |
| | | (SEQ ID NO: 175)(Kabat) | NO: 181) |
| | | CDR2: DPEQGD (SEQ ID | CDR3: QQRSNYPLT (SEQ ID |
| | | NO: 191)(Chothia) or | NO: 182) |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| 49. | Humanized | EVQLVQSGAEVVKPGATVKL | EIVLTQSPATLSASPGERATL |
| | 1A1.A3- | SCKASDFNIKDAYMHWVQQ | SCSTSSSVSYMHWFQQKPG |
| | CEACAM5- | APGKGLEWIGWIDPEQGDTE | QAPRLWIYSTSNLASGVPAR |
| | B.02-BM- | YAPKFQGRATITADTSTDTAY | FSGSGSGTDYTLTISSLEPED |
| | H1_QG-VH | LELSSLRSEDTAVYYCNVITT | FAVYYCQQRSNYPLTFGAG |
| | (Variant 2) | VVNYAMDYWGQGTLVTVSS | TKVEIK (SEQ ID NO: 184) |
| | | (SEQ ID NO: 199) | CDR1: STSSSVSYMH (SEQ |
| | | CDR1: DFNIKDA (SEQ ID | ID NO: 180) |
| | | NO: 200)(Chothia) or DAYMH | CDR2: STSNLAS (SEQ ID |
| | | (SEQ ID NO: 201)(Kabat) | NO: 181) |
| | | CDR2: DPEQGD (SEQ ID | CDR3: QQRSNYPLT (SEQ ID |
| | | NO: 191)(Chothia) or | NO: 182) |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| 50. | Humanized | EVQLVQSGAEVVKPGATVKL | EIVLTQSPATLSASPGERATL |
| | -1A1.A3- | SCKASDFNIKDXYMHWVQQ | SCSTSSSVSYMHWFQQKPG |
| | CEACAM5- | APGKGLEWIGWIDPEQGDTE | QAPRLWIYSTSNLASGVPAR |
| | B.02-BM- | YAPKFQGRATITADTSTDTAY | FSGSGSGTDYTLTISSLEPED |
| | H1_QG-VH | LELSSLRSEDTAVYYCNVITT | FAVYYCQQRSNYPLTFGAG |
| | Consensus | VVNYAMDYWGQGTLVTVSS | TKVEIK (SEQ ID NO: 184) |
| | Sequence | where X is S or A | CDR1: STSSSVSYMH (SEQ |
| | | (SEQ ID NO: 202) | ID NO: 180) |
| | | CDR1: DFNIKDX (SEQ ID | CDR2: STSNLAS (SEQ ID |
| | | NO: 194)(Chothia) or DXYMH | NO: 181) |
| | | (Kabat) where X is S or A (SEQ | CDR3: QQRSNYPLT (SEQ ID |
| | | ID NO: 195) | NO: 182) |
| | | CDR2: DPEQGD (SEQ ID | |
| | | NO: 191)(Chothia) or | |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| | 4B10.B3- | EVQLQESGAELMKPGASVKIS | |
| | CEACAM5- | CKATGYTFSTYWIEWVKPRP | |
| | A.02_VH1 | GHGLEWIGEILPGTGTTNYNE | |
| | | KFKGKATFTADTSSNTAYMQ | |
| | | LSSLTSEDSAVYYCATLNGH | |
| | | GDYWYFDVWGAGTTVTVSS | |
| | | (SEQ ID NO: 639) | |
| | | CDR1: GYTFSTY (SEQ ID | |
| | | NO: 640)(Chothia) or TYWIE | |
| | | (SEQ ID NO: 641)(Kabat) | |
| | | CDR2: LPGTGT (SEQ ID | |
| | | NO: 642)(Chothia) or | |
| | | EILPGTGTTNYNEKFKG (SEQ | |
| | | ID NO: 643)(Kabat) | |
| | | CDR3: LNGHGDYWYFDV | |
| | | (SEQ ID NO: 644) | |
| | 4B10.B3- | QVQLQQSGAELMKPGASVKI | |
| | CEACAM5- | SCKATGYTFSTYWIEWVKPR | |
| | A.02_VH2 | PGHGLEWIGEILPGTGTTNYN | |
| | | EKFKGKATFTADTSSNTAYM | |
| | | QLSSLTSEDSAVYYCATLNG | |
| | | HGDYWYFDVWGAGTTVTVS | |
| | | S (SEQ ID NO: 645) | |
| | | CDR1: GYTFSTY (SEQ ID | |
| | | NO: 640)(Chothia) or TYWIE | |
| | | (SEQ ID NO: 641)(Kabat) | |
| | | CDR2: LPGTGT (SEQ ID | |
| | | NO: 642)(Chothia) or | |
| | | EILPGTGTTNYNEKFKG (SEQ | |
| | | ID NO: 643)(Kabat) | |
| | | CDR3: LNGHGDYWYFDV | |
| | | (SEQ ID NO: 644) | |
| | 4B10.B3- | | QAVVTQESALTTSPGETVTL |
| | CEACAM5- | | TCRSSTGGVTTSNYANWVQ |
| | A.02_VL | | EKPDHLFTGLIGGTNNRAPG |
| | | | VPARFSGSLIGDKAALTITGA |
| | | | QTEDEAIYFCALWYSNHLVF |
| | | | GGGTKLTVL (SEQ ID |
| | | | NO: 646) |
| | | | CDR1: RSSTGGVTTSNYAN |
| | | | (SEQ ID NO: 647) |
| | | | CDR2: GTNNRAP (SEQ ID |
| | | | NO: 648) |
| | | | CDR3: ALWYSNHLV (SEQ ID |
| | | | NO: 649) |
| | 13C7.A6- | EVQLQESGPELVKPGASLKIS | |
| | CEACAM5- | CKASGYSFTDYTMNWVKQS | |
| | B.02_VH1 | HGKNLEWIGLINPYNGGTTY | |
| | | NQKFKDMATLTVDKSSSTAY | |
| | | MELLSLTSEDSAVYYCARSE | |
| | | YGHSYWYFDVWGAGTTVTV | |
| | | SP (SEQ ID NO: 650) | |
| | | CDR1: GYSFTDY (SEQ ID | |
| | | NO: 651)(Chothia) or DYTMN | |
| | | (SEQ ID NO: 652)(Kabat) | |
| | | CDR2: NPYNGG (SEQ ID | |
| | | NO: 653)(Chothia) or | |
| | | LINPYNGGTTYNQKFKD (SEQ | |
| | | ID NO: 654)(Kabat) | |
| | | CDR3: SEYGHSYWYFDV | |
| | | (SEQ ID NO: 655) | |
| | 13C7.A6- | EVQLQQSGPELVKPGASLKIS | |
| | CEACAM5- | CKASGYSFTDYTMNWVKQS | |
| | B.02_VH2 | HGKNLEWIGLINPYNGGTTY | |
| | | NQKFKDMATLTVDKSSSTAY | |
| | | MELLSLTSEDSAVYYCARSE | |
| | | YGHSYWYFDVWGAGTTVTV | |
| | | SS (SEQ ID NO: 656) | |
| | | CDR1: GYSFTDY (SEQ ID | |
| | | NO: 651)(Chothia) or DYTMN | |
| | | (SEQ ID NO: 652)(Kabat) | |
| | | CDR2: NPYNGG (SEQ ID | |
| | | NO: 653)(Chothia) or | |
| | | LINPYNGGTTYNQKFKD (SEQ | |
| | | ID NO: 654)(Kabat) | |
| | | CDR3: SEYGHSYWYFDV | |
| | | (SEQ ID NO: 655) | |
| | 13C7.A6- | | DIVMSQSPSSLAVSVGEKVT |
| | CEACAM5- | | MSCKSSHSLLYGNFQNNYL |
| | B.02_VL1 | | AWYQQKPGQSPKLLIYWAS |
| | | | TRESGVPDRFTGSGSGTDFT |
| | | | LTISSVKAEDLAVYYCQQYY |
| | | | SYPYTFGGGTKLEIK (SEQ |
| | | | ID NO: 106) |
| | | | CDR1: |
| | | | KSSHSLLYGNFQNNYLA |
| | | | (SEQ ID NO: 107) |
| | | | CDR2: WASTRES (SEQ ID |
| | | | NO: 108) |
| | | | CDR3: QQYYSYPYT (SEQ ID |
| | | | NO: 109) |
| | 13C7.A6- | | QIVLTQSPAIMSASPGEKVTL |
| | CEACAM5- | | TCSPSSSVTYMHWYQQKSG |
| | B.02_VL2 | | TSPKRWIYDTSKLASGVPAR |
| | | | FSGIGSGTSYSLTINSMEAED |
| | | | AATYYCQQWNNYPYTFGG |
| | | | GTRLEIK (SEQ ID NO: 657) |
| | | | CDR1: SPSSSVTYMH (SEQ |
| | | | ID NO: 658) |
| | | | CDR2: DTSKLAS (SEQ ID |
| | | | NO: 659) |
| | | | CDR3: QQWNNYPYT (SEQ |
| | | | ID NO: 660) |
| | 13C7.A6- | | DIQMTQSPASLSASVGDTVT |
| | CEACAM5- | | ITCRASENIYSYFAWYQQKQ |
| | B.02_VL3 | | GKSPRLLVYNARALAEGVPS |
| | | | RFSGSGSGTQFSKINSLQPED |
| | | | FGSYYCQHLYGAPFTFGSGT |
| | | | KLEIK (SEQ ID NO: 661) |
| | | | CDR1: RASENIYSYFA (SEQ |
| | | | ID NO: 662) |
| | | | CDR2: NARALAE (SEQ ID |
| | | | NO: 663) |
| | | | CDR3: QHLYGAPFT (SEQ ID |
| | | | NO: 664) |
| | 7E11.B2- | QIQLVQSGPELKKPGETVKIS | |
| | CEACAM5- | CKASGYSFTKYGMNWVKQA | |
| | B.02_VH1 | PGKGLKWMGWINTYSGEPTY | |
| | | ADDFEGRFAFSLETSANTAYL | |
| | | QINNLKNEDMATYFCARGGG | |
| | | FDYGFDYWGQGTTLTVST | |
| | | (SEQ ID NO: 665) | |
| | | CDR1: GYSFTKY (SEQ ID | |
| | | NO: 666)(Chothia) or KYGMN | |
| | | (SEQ ID NO: 667)(Kabat) | |
| | | CDR2: NTYSGE (SEQ ID | |
| | | NO: 668)(Chothia) or | |
| | | WINTYSGEPTYADDFEG (SEQ | |
| | | ID NO: 669)(Kabat) | |
| | | CDR3: GGGFDYGFDY (SEQ | |
| | | ID NO: 670) | |
| | 7E11.B2- | QIQLVQSGPELKKPGETVKIS | |
| | CEACAM5- | CKASGYSFTKYGMNWVKQA | |
| | B.02_VH2 | PGKGLKWMGWINTYSGEPTY | |
| | | ADDFEGRFAFSLETSANTAYL | |
| | | QINNLKNEDMATYFCARGGG | |
| | | FDYGFDYWGQGTTLTVSS | |
| | | (SEQ ID NO: 699) | |
| | | CDR1: GYSFTKY (SEQ ID | |
| | | NO: 666)(Chothia) or KYGMN | |
| | | (SEQ ID NO: 667)(Kabat) | |
| | | CDR2: NTYSGE (SEQ ID | |
| | | NO: 668)(Chothia) or | |
| | | WINTYSGEPTYADDFEG (SEQ | |
| | | ID NO: 669)(Kabat) | |
| | | CDR3: GGGFDYGFDY (SEQ | |
| | | ID NO: 670) | |
| | 7E11.B2- | | QIVLTQSPAIMSASPGEKVT |
| | CEACAM5- | | MTCSASSSVSYIHWYRQRSG |
| | B.02_VL | | TSPKRWIYDTSKLASGVPAR |
| | | | FSGSGSGTSYSLTISTMEAED |
| | | | AATYYCQQWSNYPYTFGGG |
| | | | TKLEIK (SEQ ID NO: 671) |
| | | | CDR1: SASSSVSYIH (SEQ ID |
| | | | NO: 672) |
| | | | CDR2: DTSKLAS (SEQ ID |
| | | | NO: 659) |
| | | | CDR3: QQWSNYPYT (SEQ ID |
| | | | NO: 673) |
| | 10D6.E3- | EVQLQQSGPELVKPGASMKIS | |
| | CEACAM5- | CKASGYSFTDYTMNWVKQS | |
| | B.02_VH | HGKNLEWIGHIYPYNGGTTY | |
| | | NQKFQDKASLTADKSSSTAY | |
| | | MELLSLTSEDSAVYYCARGE | |
| | | FLRSYWYFDVWGAGTLVAV | |
| | | SS (SEQ ID NO: 674) | |
| | | CDR1: GYSFTDY (SEQ ID | |
| | | NO: 651)(Chothia) or DYTMN | |
| | | (SEQ ID NO: 652)(Kabat) | |
| | | CDR2: YPYNGG (SEQ ID | |
| | | NO: 675)(Chothia) or | |
| | | HIYPYNGGTTYNQKFQD | |
| | | (SEQ ID NO: 676)(Kabat) | |
| | | CDR3: GEFLRSYWYFDV (SEQ | |
| | | ID NO: 677) | |
| | 10D6.E3- | | QIVLTQSPAIMSASPGEKVTL |
| | CEACAM5- | | TCSPSSSVTYMHWYQQKSG |
| | B.02_VL1 | | TSPKRWIYDTSKLASGVPAR |
| | | | FSGIGSGTSYSLTINSMEAED |
| | | | AATYYCQQWNNYPYTFGG |
| | | | GTKLEIK (SEQ ID NO: 678) |
| | | | CDR1: SPSSSVTYMH (SEQ |
| | | | ID NO: 658) |
| | | | CDR2: DTSKLAS (SEQ ID |
| | | | NO: 659) |
| | | | CDR3: QQWNNYPYT (SEQ |
| | | | ID NO: 660) |
| | 10D6.E3- | | DIQMTQSPASLSASVGETVTI |
| | CEACAM5- | | TCRGSENIYSYLTWYQQKQ |
| | B.02_VL2 | | GKSPQLLVYNAKTLAEGVPS |
| | | | RFSGSGSGTQFSLKINSLQPE |
| | | | DFGRYYCQHLYSSPYTFGG |
| | | | GTKLEIK (SEQ ID NO: 679) |
| | | | CDR1: RGSENIYSYLT (SEQ |
| | | | ID NO: 680) |
| | | | CDR2: NAKTLAE (SEQ ID |
| | | | NO: 681) |
| | | | CDR3: QHLYSSPYT (SEQ ID |
| | | | NO: 682) |
| | 13C7.F2- | EVQLQQSGPELVKPGASLKIS | |
| | CEACAM5- | CKASGYSFTDYTMNWVKQS | |
| | B.02_VH | HGKNLEWIGLINPYNGGTTY | |
| | | NQKFKDMATLTVDKSSSTAY | |
| | | MELLSLTSEDSAVYYCARSE | |
| | | YGHSYWYFDVWGAGTTVTV | |
| | | SS (SEQ ID NO: 656) | |
| | | CDR1: GYSFTDY (SEQ ID | |
| | | NO: 651)(Chothia) or DYTMN | |
| | | (SEQ ID NO: 652)(Kabat) | |
| | | CDR2: NPYNGG (SEQ ID | |
| | | NO: 653)(Chothia) or | |
| | | LINPYNGGTTYNQKFKD (SEQ | |
| | | ID NO: 654)(Kabat) | |
| | | CDR3: SEYGHSYWYFDV | |
| | | (SEQ ID NO: 655) | |
| | 13C7.F2- | | QIVLTQSPAIMSASPGEKVTL |
| | CEACAM5- | | TCSPSSSVTYMHWYQQKSG |
| | B.02_VL1 | | TSPKRWIYDTSKLASGVPAR |
| | | | FSGIGSGTSYSLTINSMEAED |
| | | | AATYYCQQWNNYPYTFGG |
| | | | GTRLEIK (SEQ ID NO: 657) |
| | | | CDR1: SPSSSVTYMH (SEQ |
| | | | ID NO: 658) |
| | | | CDR2: DTSKLAS (SEQ ID |
| | | | NO: 659) |
| | | | CDR3: QQWNNYPYT (SEQ |
| | | | ID NO: 660) |
| | 13C7.F2- | | DIQMTQSPASLSASVGDTVT |
| | CEACAM5- | | ITCRASENIYSYFAWYQQKQ |
| | B.02_VL2 | | GKSPRLLVYNARALAEGVPS |
| | | | RFSGSGSGTQFSLKINSLQPE |
| | | | DFGSYYCQHLYGAPFTFGSG |
| | | | TKLEIK (SEQ ID NO: 683) |
| | | | CDR1: RASENIYSYFA (SEQ |
| | | | ID NO: 662) |
| | | | CDR2: NARALAE (SEQ ID |
| | | | NO: 663) |
| | | | CDR3: QHLYGAPFT (SEQ ID |
| | | | NO: 664) |
| 51. | 16B11.G2- | QVQLQESGPGLVTPSGTLSLT | EIVMTQSPATLSVSPGERAT |
| | CEACAM5- | CAVSGDSISSSHWWSWVRQP | LSCRASQSVRSNLAWYLQK |
| | B.01_VH | PGKGLEWIGEIYHSGITNYRS | PGQAPRLLIYGASTRATGIPA |
| | and | SLKSRVTLSVDKSKNQFSLKL | RFSGSGSGTEFTLTISSLQSE |
| | 16B11.G2- | TSVTAADTAVYYCARGGSGN | DFAVYYCQQYNNWPLTFGG |
| | CEACAM5- | YEAFDIWGQGTLFTVSS (SEQ | GTKVEIK (SEQ ID NO: 690) |
| | B.01_VL | ID NO: 684) | CDR1: RASQSVRSNLA (SEQ |
| | | CDR1: GDSISSSH (SEQ ID | ID NO: 62) |
| | | NO: 685) | CDR2: GASTRAT (SEQ ID |
| | | (Chothia) or SSHWWS (SEQ ID | NO: 63) |
| | | NO: 686)(Kabat) | CDR3: QQYNNWPLT (SEQ ID |
| | | CDR2: YHSGI (SEQ ID NO: 687) | NO: 42) |
| | | (Chothia) or | |
| | | EIYHSGITNYRSSLKS (SEQ ID | |
| | | NO: 688)(Kabat) | |
| | | CDR3: GGSGNYEAFDI (SEQ | |
| | | ID NO: 689) | |

Tier 3

| | | | |
|---|---|---|---|
| 52. | 1080_G01- | QVQLVESGGGVVQSGRSLRL | EIVLTQSPSTLSASVGDRVTI |
| | CEACAM5 | SCAASGFMFSNFAMHWVRQ | TCRASQSISNWLAWYQQKP |
| | | APGKGLEWVGVIWYDGSNK | GKAPKLLIYKASSLESGVPS |
| | | FYADSVKGRFTISRDNSKNTL | RFSGSGSGTEFTLTISSLQPD |
| | | NLQMSRLRAEDTAVYYCAR | DFATYYCQQYNSYSYTFGQ |
| | | DGREVWRYYHYGMDVWGQ | GTKVEIK (SEQ ID NO: 208) |
| | | GTTVTVSS (SEQ ID NO: 377) | CDR1: RASQSISNWLA (SEQ |
| | | CDR1: GFMFSNF (SEQ ID | ID NO: 209) |
| | | NO: 204)(Chothia) or NFAMH | CDR2: KASSLES (SEQ ID |
| | | (SEQ ID NO: 205)(Kabat) | NO: 8) |
| | | CDR2: WYDGSN (SEQ ID | CDR3: QQYNSYSYT (SEQ ID |
| | | NO: 70)(Chothia) or | NO: 9) |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 207) | |
| | 1080_G01- | | EIVLTQSPSTLSASVGDRVTI |
| | CEACAM5_ | | TCRASQSISNWLAWYQQKP |
| | VL Variant | | GKAPKLLIYKASSLESGVPS |
| | | | RFSGSGSGTEFTLTISSLQPD |
| | | | DFATYYCQQYNAYSYTFGQ |
| | | | GTKVEIK (SEQ ID NO: 210) |
| | | | CDR1: RASQSISNWLA (SEQ |
| | | | ID NO: 209) |
| | | | CDR2: KASSLES (SEQ ID |
| | | | NO: 8) |
| | | | CDR3: QQYNAYSYT (SEQ ID |
| | | | NO: 27) |
| | VH | EVQLVESGGGVVQSGRSLRL | |
| | 1080_G01- | SCAASGFMFSNFAMHWVRQ | |
| | CEACAM5_ | APGKGLEWVGVIWYDGSNK | |
| | (QH1E) | FYADSVKGRFTISRDNSKNTL | |
| | | NLQMSRLRAEDTAVYYCAR | |
| | | DGREVWRYYHYGMDVWGQ | |
| | | GTTVTVSS (SEQ ID NO: 357) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 203) | |
| | VH | QVQLVESGGGVVQSGRSLRL | |
| | 1080_G01- | SCAASGFMFSNFAMHWVRQ | |
| | CEACAM5_ | APGKGLEWVGVIWYDASNK | |
| | (GH54A) | FYADSVKGRFTISRDNSKNTL | |
| | | NLQMSRLRAEDTAVYYCAR | |
| | | DGREVWRYYHYGMDVWGQ | |
| | | GTTVTVSS (SEQ ID NO: 358) | |
| | | CDR1: GFMFSNF (SEQ ID | |
| | | NO: 204)(Chothia) or NFAMH | |
| | | (SEQ ID NO: 205)(Kabat) | |
| | | CDR2: WYDASN (SEQ ID | |
| | | NO: 359)(Chothia) or | |
| | | VIWYDASNKFYADSVKG | |
| | | (SEQ ID NO: 360)(Kabat) | |
| | | CDR3: | |
| | | DGREVWRYYHYGMDV (SEQ | |
| | | ID NO: 203) | |
| | VH | QVQLVESGGGVVQSGRSLRL | |
| | 1080_G01- | SCAASGFMFSNFAMHWVRQ | |
| | CEACAM5_ | APGKGLEWVGVIWYDGSNK | |
| | (SH62A) | FYADAVKGRFTISRDNSKNTL | |
| | | NLQMSRLRAEDTAVYYCAR | |
| | | DGREVWRYYHYGMDVWGQ | |
| | | GTTVTVSS (SEQ ID NO: 361) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADAVKG | |
| | | (SEQ ID NO: 362)(Kabat) | |
| | | CDR3: | |
| | | DGREVWRYYHYGMDV (SEQ | |
| | | ID NO: 207) | |
| | VH | QVQLVESGGGVVQSGRSLRL | |
| | 1080_G01- | SCAASGFMFSNFAMHWVRQ | |
| | CEACAM5_ | APGKGLEWVGVIWYDGSNK | |
| | (NH73A) | FYADSVKGRFTISRDASKNTL | |
| | | NLQMSRLRAEDTAVYYCAR | |
| | | DGREVWRYYHYGMDVWGQ | |
| | | GTTVTVSS (SEQ ID NO: 363) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 207) | |
| | VH | QVQLVESGGGVVQSGRSLRL | |
| | 1080_G01- | SCAASGFMFSNFAMHWVRQ | |
| | CEACAM5_ | APGKGLEWVGVIWYDGSNK | |
| | (SH74A) | FYADSVKGRFTISRDNAKNTL | |
| | | NLQMSRLRAEDTAVYYCAR | |
| | | DGREVWRYYHYGMDVWGQ | |
| | | GTTVTVSS (SEQ ID NO: 364) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 207) | |
| | VH | QVQLVESGGGVVQSGRSLRL | |
| | 1080_G01- | SCAASGFMFSNFAMHWVRQ | |
| | CEACAM5_ | APGKGLEWVGVIWYDGSNK | |
| | (GH96A) | FYADSVKGRFTISRDNSKNTL | |
| | | NLQMSRLRAEDTAVYYCAR | |
| | | DAREVWRYYHYGMDVWGQ | |
| | | GTTVTVSS (SEQ ID NO: 365) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DAREVWRYYHYGMDV (SEQ | |
| | | ID NO: 366) | |
| 53. | 1080_G01- | X₁VQLVESGGGVVQSGRSLRL | EIVLTQSPSTLSASVGDRVTI |
| | CEACAM5 | SCAASGFMFSNFAMHWVRQ | TCRASQSISNWLAWYQQKP |
| | Consensus | APGKGLEWVGVIWYDX₂SNK | GKAPKLLIYKASSLESGVPS |
| | Sequence | FYADX₃VKGRFTISRDX₄X₅KN | RFSGSGSGTEFTLTISSLQPD |
| | | TLNLQMSRLRAEDTAVYYCA | DFATYYCQQYNXYSYTFGQ |
| | | RDX₆REVWRYYHYGMDVWG | GTKVEIK |
| | | QGTTVTVSS | where X is S or A |
| | | where X₁ is Q or E, X₂ is G or A, | (SEQ ID NO: 215) |
| | | and X₃ is S or A, X₄ is N or A, X₅ | CDR1: RASQSISNWLA (SEQ |
| | | is S or A, and X₆ is G and A | ID NO: 209) |
| | | (SEQ ID NO: 211) | CDR2: KASSLES (SEQ ID |
| | | CDR1: GFMFSNF (SEQ ID | NO: 8) |
| | | NO: 204)(Chothia) or NFAMH | CDR3: QQYNXYSYT where X |
| | | (SEQ ID NO: 205)(Kabat) | is S or A (SEQ ID NO: 33) |
| | | CDR2: WYDXSN (SEQ ID | |
| | | NO: 212)(Chothia) or | |
| | | VIWYDXSNKFYADSVKG | |
| | | (Kabat) where X is G or A (SEQ | |
| | | ID NO: 213) | |
| | | CDR3: | |
| | | DXREVWRYYHYGMDV where | |
| | | X is G or A (SEQ ID NO: 214) | |
| 54. | 1078_C12- | QVQLVQSGAEVKKPGASVKV | DIQLTQSPSSLSASVGDRVTI |
| | CEACAM5 | SCTASGYTFTGYFIHWVRQAP | TCRASQSISSYLNWYQQKPG |
| | | GQGLEWMGWINPHSGATNY | KAPKLLIYAASSLQSGVPSRF |
| | | AQKFQGRVTMTRDTSISTAY | SGSGSGTDFTLTISSLQPEDF |
| | | MELSSLRSDDTAVYYCARVS | ATYYCQQSYGNPLTFGGGT |
| | | YYGLDVWGQGTTVTVSS | KVEIK (SEQ ID NO: 222) |
| | | (SEQ ID NO: 216) | CDR1: RASQSISSYLN (SEQ |
| | | CDR1: GYTFTGY (SEQ ID | ID NO: 223) |
| | | NO: 217)(Chothia) or GYFIH | CDR2: AASSLQS (SEQ ID |
| | | (SEQ ID NO: 218)(Kabat) | NO: 224) |
| | | CDR2: NPHSGA (SEQ ID | CDR3: QQSYGNPLT (SEQ ID |
| | | NO: 219)(Chothia) or | NO: 225) |
| | | WINPHSGATNYAQKFQG | |
| | | (SEQ ID NO: 220)(Kabat) | |
| | | CDR3: VSYYGLDV (SEQ ID | |
| | | NO: 221) | |
| | VH_1078_ | EVQLVQSGAEVKKPGASVKV | |
| | C12- | SCTASGYTFTGYFIHWVRQAP | |
| | CEACAM5 | GQGLEWMGWINPHSGATNY | |
| | (QH1E) | AQKFQGRVTMTRDTSISTAY | |
| | | MELSSLRSDDTAVYYCARVS | |
| | | YYGLDVWGQGTTVTVSS | |
| | | (SEQ ID NO: 367) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| | VH_1078_ | QVQLVQSGAEVKKPGASVKV | |
| | C12- | SCTASGYTFTGYFIHWVRQAP | |
| | CEACAM5 | GQGLEWIGWINPHSGATNYA | |
| | (MH48I) | QKFQGRVTMTRDTSISTAYM | |
| | | ELSSLRSDDTAVYYCARVSY | |
| | | YGLDVWGQGTTVTVSS (SEQ | |
| | | ID NO: 368) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| | VH_1078_ | QVQLVQSGAEVKKPGASVKV | |
| | C12- | SCTASGYTFTGYFIHWVRQAP | |
| | CEACAM5 | GQGLEWVGWINPHSGATNY | |
| | (MH48V) | AQKFQGRVTMTRDTSISTAY | |
| | | MELSSLRSDDTAVYYCARVS | |
| | | YYGLDVWGQGTTVTVSS | |
| | | (SEQ ID NO: 369) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| | VH_1078_ | QVQLVQSGAEVKKPGASVKV | |
| | C12- | SCTASGYTFTGYFIHWVRQAP | |
| | CEACAM5 | GQGLEWMGWINPHSGATNY | |
| | (MH69I) | AQKFQGRVTITRDTSISTAYM | |
| | | ELSSLRSDDTAVYYCARVSY | |
| | | YGLDVWGQGTTVTVSS (SEQ | |
| | | ID NO: 370) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| | VH_1078_ | QVQLVQSGAEVKKPGASVKV | |
| | C12- | SCTASGYTFTGYFIHWVRQAP | |
| | CEACAM5 | GQGLEWMGWINPHSGATNY | |
| | (MH69V) | AQKFQGRVTVTRDTSISTAY | |
| | | MELSSLRSDDTAVYYCARVS | |
| | | YYGLDVWGQGTTVTVSS | |
| | | (SEQ ID NO: 371) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 55. | 1078_C12- | X₁VQLVQSGAEVKKPGASVK | DIQLTQSPSSLSASVGDRVTI |
| | CEACAM5 | VSCTASGYTFTGYFIHWVRQ | TCRASQSISSYLNWYQQKPG |
| | Consensus | APGQGLEWX₂GWINPHSGAT | KAPKLLIYAASSLQSGVPSRF |
| | Sequence | NYAQKFQGRVTX₃TRDTSIST | SGSGSGTDFTLTISSLQPEDF |
| | | AYMELSSLRSDDTAVYYCAR | ATYYCQQSYGNPLTFGGGT |
| | | VSYYGLDVWGQGTTVTVSS | KVEIK (SEQ ID NO: 222) |
| | | where X₁ is Q or E, where X₂ is | CDR1: RASQSISSYLN (SEQ |
| | | M, V or I, and X₃ is M, V, or I | ID NO: 223) |
| | | (SEQ ID NO: 226) | CDR2: AASSLQS (SEQ ID |
| | | CDR1: GYTFTGY (SEQ ID | NO: 224) |
| | | NO: 217)(Chothia) or GYFIH | CDR3: QQSYGNPLT (SEQ ID |
| | | (SEQ ID NO: 218)(Kabat) | NO: 225) |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 56. | 1078_F02- | QVQLQESGAEVKKPGASVKV | DIVLTQTPSSLSASVGDRVTI |
| | CEACAM5 | SCKASGYTFTGYYLHWVRQA | TCWASQSISSYLNWYQQKP |
| | | PGQGLEWMGWINPNSGDTN | GKAPKLLIYAASSLQSGVPS |
| | | YAQKFQGRVTMTRDTSISTA | RFSGSGSGTDFTLTISSLQPE |
| | | YMELSRLRSDDTAVYYCARV | DFATYYCQQSYSTPLTFGGG |
| | | SYYGLDVWGQGTTVTVSS | TKLEIK (SEQ ID NO: 231) |
| | | (SEQ ID NO: 227) | CDR1: WASQSISSYLN (SEQ |
| | | CDR1: GYTFTGY (SEQ ID | ID NO: 232) |
| | | NO: 217)(Chothia) or GYYLH | CDR2: AASSLQS (SEQ ID |
| | | (SEQ ID NO: 228)(Kabat) | NO: 224) |
| | | CDR2: NPNSGD (SEQ ID | CDR3: QQSYSTPLT (SEQ ID |
| | | NO: 229)(Chothia) or | NO: 233) |
| | | WINPNSGDTNYAQKFQG | |
| | | (SEQ ID NO: 230)(Kabat) | |
| | | CDR3: VSYYGLDV (SEQ ID | |
| | | NO: 221) | |
| 57. | 1079_B08- | QVQLVQSGAEVKKPGASVKV | EIVLTQSPSSLSASVGDRVTI |
| | CEACAM5 | SCKASGYTFTGYYLHWVRQA | TCRASQSISSYLNWYQQKPG |
| | | PGQGLEWMGWINPSSGDTNY | KAPKLLIYAASSLQSGVPSRF |
| | | AQKFQGRVTMTRDTSISTAY | SGSGSGTDFTLTISSLQPEDF |
| | | MELSRLRSDDTAVYYCARVS | ATYYCQQSYSNPLTFGGGT |
| | | YYGLDVWGQGTTVTVSS | KVDIK (SEQ ID NO: 237) |
| | | (SEQ ID NO: 234) | CDR1: RASQSISSYLN (SEQ |
| | | CDR1: GYTFTGY (SEQ ID | ID NO: 223) |
| | | NO: 217)(Chothia) or GYYLH | CDR2: AASSLQS (SEQ ID |
| | | (SEQ ID NO: 228)(Kabat) | NO: 224) |
| | | CDR2: NPSSGD (SEQ ID | CDR3: QQSYSNPLT (SEQ ID |
| | | NO: 235)(Chothia) or | NO: 238) |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH_1078_F02- | EVQLQESGAEVKKPGASVKV | |
| | CEACAM5 | SCKASGYTFTGYYLHWVRQA | |
| | (QH1E) | PGQGLEWMGWINPNSGDTN | |
| | | YAQKFQGRVTMTRDTSISTA | |
| | | YMELSRLRSDDTAVYYCARV | |
| | | SYYGLDVWGQGTTVTVSS | |
| | | (SEQ ID NO: 372) | |
| | | CDR1: GYTFTGY (SEQ ID | |
| | | NO: 217)(Chothia) or GYYLH | |
| | | (SEQ ID NO: 228)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (N5H3S) | PGQGLEWMGWINPSSGDTNY | |
| | | AQKFQGRVTMTRDTSISTAY | |
| | | MELSRLRSDDTAVYYCARVS | |
| | | YYGLDVWGQGTTVTVSS | |
| | | (SEQ ID NO: 373) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (SH54A) | PGQGLEWMGWINPNAGDTN | |
| | | YAQKFQGRVTMTRDTSISTA | |
| | | YMELSRLRSDDTAVYYCARV | |
| | | SYYGLDVWGQGTTVTVSS | |
| | | (SEQ ID NO: 374) | |
| | | CDR1: GYTFTGY (SEQ ID | |
| | | NO: 217)(Chothia) or GYYLH | |
| | | (SEQ ID NO: 228)(Kabat) | |
| | | CDR2: NPNAGD (SEQ ID | |
| | | NO: 375)(Chothia) or | |
| | | WINPNAGDTNYAQKFQG | |
| | | (SEQ ID NO: 376)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (MH48I) | PGQGLEWIGWINPNSGDTNY | |
| | | AQKFQGRVTMTRDTSISTAY | |
| | | MELSRLRSDDTAVYYCARVS | |
| | | YYGLDVWGQGTTVTVSS | |
| | | (SEQ ID NO: 378) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (MH48V) | PGQGLEWVGWINPNSGDTNY | |
| | | AQKFQGRVTMTRDTSISTAY | |
| | | MELSRLRSDDTAVYYCARVS | |
| | | YYGLDVWGQGTTVTVSS | |
| | | (SEQ ID NO: 379) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (MH69I) | PGQGLEWMGWINPNSGDTN | |
| | | YAQKFQGRVTITRDTSISTAY | |
| | | MELSRLRSDDTAVYYCARVS | |
| | | YYGLDVWGQGTTVTVSS | |
| | | (SEQ ID NO: 380) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (MH69V) | PGQGLEWMGWINPNSGDTN | |
| | | YAQKFQGRVTVTRDTSISTAY | |
| | | MELSRLRSDDTAVYYCARVS | |
| | | YYGLDVWGQGTTVTVSS | |
| | | (SEQ ID NO: 381) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (MH80I) | PGQGLEWMGWINPNSGDTN | |
| | | YAQKFQGRVTMTRDTSISTA | |
| | | YIELSRLRSDDTAVYYCARVS | |
| | | YYGLDVWGQGTTVTVSS | |
| | | (SEQ ID NO: 382) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| | VH1078_F02- | QVQLQESGAEVKKPGASVKV | |
| | CEACAM5 | SCKASGYTFTGYYLHWVRQA | |
| | (MH80V) | PGQGLEWMGWINPNSGDTN | |
| | | YAQKFQGRVTMTRDTSISTA | |
| | | YVELSRLRSDDTAVYYCARV | |
| | | SYYGLDVWGQGTTVTVSS | |
| | | (SEQ ID NO: 383) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| | VH | EVQLVQSGAEVKKPGASVKV | |
| | 1079_B08- | SCKASGYTFTGYYLHWVRQA | |
| | CEACAM5 | PGQGLEWMGWINPSSGDTNY | |
| | (QH1E) | AQKFQGRVTMTRDTSISTAY | |
| | | MELSRLRSDDTAVYYCARVS | |
| | | YYGLDVWGQGTTVTVSS | |
| | | (SEQ ID NO: 384) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH | QVQLVQSGAEVKKPGASVKV | |
| | 1079_B08- | SCKASGYTFTGYYLHWVRQA | |
| | CEACAM5 | PGQGLEWIGWINPSSGDTNY | |
| | (MH48I) | AQKFQGRVTMTRDTSISTAY | |
| | | MELSRLRSDDTAVYYCARVS | |
| | | YYGLDVWGQGTTVTVSS | |
| | | (SEQ ID NO: 385) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH | QVQLVQSGAEVKKPGASVKV | |
| | 1079_B08- | SCKASGYTFTGYYLHWVRQA | |
| | CEACAM5 | PGQGLEWVGWINPSSGDTNY | |
| | (MH48V) | AQKFQGRVTMTRDTSISTAY | |
| | | MELSRLRSDDTAVYYCARVS | |
| | | YYGLDVWGQGTTVTVSS | |
| | | (SEQ ID NO: 386) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH | QVQLVQSGAEVKKPGASVKV | |
| | 1079_B08- | SCKASGYTFTGYYLHWVRQA | |
| | CEACAM5 | PGQGLEWMGWINPSSGDTNY | |
| | (MH69I) | AQKFQGRVTITRDTSISTAYM | |
| | | ELSRLRSDDTAVYYCARVSY | |
| | | YGLDVWGQGTTVTVSS (SEQ | |
| | | ID NO: 387) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH | QVQLVQSGAEVKKPGASVKV | |
| | 1079_B08- | SCKASGYTFTGYYLHWVRQA | |
| | CEACAM5 | PGQGLEWMGWINPSSGDTNY | |
| | (MH69V) | AQKFQGRVTVTRDTSISTAY | |
| | | MELSRLRSDDTAVYYCARVS | |
| | | YYGLDVWGQGTTVTVSS | |
| | | (SEQ ID NO: 388) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH | QVQLVQSGAEVKKPGASVKV | |
| | 1079_B08- | SCKASGYTFTGYYLHWVRQA | |
| | CEACAM5 | PGQGLEWMGWINPSSGDTNY | |
| | (MH80I) | AQKFQGRVTMTRDTSISTAYI | |
| | | ELSRLRSDDTAVYYCARVSY | |
| | | YGLDVWGQGTTVTVSS (SEQ | |
| | | ID NO: 389) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| | VH | QVQLVQSGAEVKKPGASVKV | |
| | 1079_B08- | SCKASGYTFTGYYLHWVRQA | |
| | CEACAM5 | PGQGLEWMGWINPSSGDTNY | |
| | (MH80V) | AQKFQGRVTMTRDTSISTAY | |
| | | VELSRLRSDDTAVYYCARVS | |
| | | YYGLDVWGQGTTVTVSS | |
| | | (SEQ ID NO: 390) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 58. | VH | X₁VQLX₂X₃SGAEVKKPGASV | DIVLTQTPSSLSASVGDRVTI |
| | Consensus | KVSCKASGYTFTGYYLHWVR | TCWASQSISSYLNWYQQKP |
| | Sequence | QAPGQGLEWX₄GWINPX₅X6G | GKAPKLLIYAASSLQSGVPS |
| | [1078_F02- | DTNYAQKFQGRVTX₇TRDTSI | RFSGSGSGTDFTLTISSLQPE |
| | CEACAM5; | STAYX₈ELSRLRSDDTAVYYC | DFATYYCQQSYSTPLTFGGG |
| | 1079_B08- | ARVSYYGLDVWGQGTTVTV | TKLEIK (SEQ ID NO: 231) |
| | CEACAM5] | SS | CDR1: WASQSISSYLN (SEQ |
| | and | where X₁ is Q or E, X₂ is Q or V, | ID NO: 232) |
| | VL | X₃ is E or Q, X₄ is M, V or I, X₅ | CDR2: AASSLQS (SEQ ID |
| | [1078_F02- | is N or S, X₆ is S or A, X₇ is M, V | NO: 224) |
| | CEACAM5] | or I, and X₈ is M, V, or I | CDR3: QQSYSTPLT (SEQ ID |
| | | (SEQ ID NO: 239) | NO: 233) |
| | | CDR1: GYTFTGY (SEQ ID | |
| | | NO: 217)(Chothia) or GYYLH | |
| | | (SEQ ID NO: 228)(Kabat) | |
| | | CDR2: NPX₁X₂GD (SEQ ID | |
| | | NO: 240)(Chothia) or | |
| | | WINPX₁X₂GDTNYAQKFQG | |
| | | (Kabat) where X₁ is N or S and | |
| | | X₂ is S or A (SEQ ID NO: 241) | |
| | | CDR3: VSYYGLDV (SEQ ID | |
| | | NO: 221) | |
| 59. | VH | X₁VQLX₂X₃SGAEVKKPGASV | EIVLTQSPSSLSASVGDRVTI |
| | Consensus | KVSCKASGYTFTGYYLHWVR | TCRASQSISSYLNWYQQKPG |
| | Sequence | QAPGQGLEWX₄GWINPX₅X₆G | KAPKLLIYAASSLQSGVPSRF |
| | [1078_F02- | DTNYAQKFQGRVTX₇TRDTSI | SGSGSGTDFTLTISSLQPEDF |
| | CEACAM5; | STAYX₈ELSRLRSDDTAVYYC | ATYYCQQSYSNPLTFGGGT |
| | 1079_B08- | ARVSYYGLDVWGQGTTVTV | KVDIK (SEQ ID NO: 237) |
| | CEACAM5] | SS | CDR1: RASQSISSYLN (SEQ |
| | and | where X₁ is Q or E, X₂ is Q or V, | ID NO: 223) |
| | VL | X₃ is E or Q, X₄ is M, V or I, X₅ | CDR2: AASSLQS (SEQ ID |
| | [1079_B08- | is N or S, and X₆ is S or A, X₇ is | NO: 224) |
| | CEACAM5] | M, V or I, and X₈ is M, V, or I | CDR3: QQSYSNPLT (SEQ ID |
| | | (SEQ ID NO: 239) | NO: 238) |
| | | CDR1: GYTFTGY (SEQ ID | |
| | | NO: 217)(Chothia) or GYYLH | |
| | | (SEQ ID NO: 228)(Kabat) | |
| | | CDR2: NPX₁X₂GD (SEQ ID | |
| | | NO: 240)(Chothia) or | |
| | | WINPX₁X₂GDTNYAQKFQG | |
| | | (Kabat) where X₁ is N or S and | |
| | | X₂ is S or A (SEQ ID NO: 241) | |
| | | CDR3: VSYYGLDV (SEQ ID | |
| | | NO: 221) | |
| 60. | 1078_G03- | QVQLVESGGGLVQPGGSLRL | DIRMTQSPSTLSASVGDRVTI |
| | CEACAM5 | SCAASGFTFSSYAMSWVRQA | TCRASQSISSWLAWYQQKP |
| | | PGKGLEWVSEISGSGDRTSYA | GKAPKLLIYKASSLESGVPS |
| | | DSVKGRFTISRDNSKNRLYLQ | RFSGSGSGTEFTLTISSLQPD |
| | | MNRLRTEDTAVYYCAKDLGP | DFATYYCQQYNSYSTWTFG |
| | | SGWYGLFDYWGQGTLVTVS | QGTKVDIK (SEQ ID NO: 164) |
| | | S (SEQ ID NO: 160) | CDR1: RASQSISSWLA (SEQ |
| | | CDR1: GFTFSSY (SEQ ID | ID NO: 165) |
| | | NO: 2)(Chothia) or SYAMS | CDR2: KASSLES (SEQ ID |
| | | (SEQ ID NO: 3)(Kabat) | NO: 8) |
| | | CDR2: SGSGDR (SEQ ID | CDR3: QQYNSYSTWT (SEQ |
| | | NO: 161)(Chothia) or | ID NO: 166) |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| | VH | EVQLVESGGGLVQPGGSLRL | |
| | 1078_G03- | SCAASGFTFSSYAMSWVRQA | |
| | CEACAM5 | PGKGLEWVSEISGSGDRTSYA | |
| | (QH1E) | DSVKGRFTISRDNSKNRLYLQ | |
| | | MNRLRTEDTAVYYCAKDLGP | |
| | | SGWYGLFDYWGQGTLVTVS | |
| | | S (SEQ ID NO: 352) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| | VH | QVQLVESGGGLVQPGGSLRL | |
| | 1078_G03- | SCAASGFTFSSYAMSWVRQA | |
| | CEACAM5 | PGKGLEWVSEISGSGDRTSYA | |
| | (SH62A) | DAVKGRFTISRDNSKNRLYL | |
| | | QMNRLRTEDTAVYYCAKDL | |
| | | GPSGWYGLFDYWGQGTLVT | |
| | | VSS (SEQ ID NO: 353) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| 61. | 1078_G03- | X₁VQLVESGGGLVQPGGSLRL | DIRMTQSPSTLSASVGDRVTI |
| | CEACAM5 | SCAASGFTFSSYAMSWVRQA | TCRASQSISSWLAWYQQKP |
| | Consensus | PGKGLEWVSEISGSGDRTSYA | GKAPKLLIYKASSLESGVPS |
| | Sequence | DX₂VKGRFTISRDNSKNRLYL | RFSGSGSGTEFTLTISSLQPD |
| | | QMNRLRTEDTAVYYCAKDL | DFATYYCQQYNSYSTWTFG |
| | | GPSGWYGLFDYWGQGTLVT | QGTKVDIK (SEQ ID NO: 164) |
| | | VSS | CDR1: RASQSISSWLA (SEQ |
| | | where X₁ is Q or E and X₂ is S or | ID NO: 165) |
| | | A | CDR2: KASSLES (SEQ ID |
| | | (SEQ ID NO: 242) | NO: 8) |
| | | CDR1: GFTFSSY (SEQ ID | CDR3: QQYNSYSTWT (SEQ |
| | | NO: 2)(Chothia) or SYAMS | ID NO: 166) |
| | | (SEQ ID NO: 3)(Kabat) | |
| | | CDR2: SGSGDR (SEQ ID | |
| | | NO: 161)(Chothia) or | |
| | | EISGSGDRTSYADXVKG | |
| | | (Kabat) where X is S or A (SEQ | |
| | | ID NO: 170) | |
| | | CDR3: DLGPSGWYGLFDY | |
| | | (SEQ ID NO: 163) | |
| 62. | 1079_A10- | QVQLVESGAEVKKPGASVKV | DIRMTQSPSSLSASVGDRVTI |
| | CEACAM5 | SCKASGYTFTGYYLHWVRQA | ICRASQSISSYLNWYQQKPG |
| | | PGQGLEWMGWINPNNGDTN | KAPKLLIYAASSLQSGVPSRF |
| | | YAQKFQGRVTMTRDTSISTA | SGSGSGTDFTLTISSLQPEDF |
| | | YMELSRLRSDDTAVYYCARV | ATYYCQQSYSNPLTFGGGT |
| | | SYYGLDVWGQGTTVTVSS | KVEIK (SEQ ID NO: 246) |
| | | (SEQ ID NO: 243) | CDR1: RASQSISSYLN (SEQ |
| | | CDR1: GYTFTGY (SEQ ID | ID NO: 223) |
| | | NO: 217)(Chothia) or GYYLH | CDR2: AASSLQS (SEQ ID |
| | | (SEQ ID NO: 228)(Kabat) | NO: 224) |
| | | CDR2: NPNNGD (SEQ ID | CDR3: QQSYSNPLT (SEQ ID |
| | | NO: 244)(Chothia) or | NO: 238) |
| | | WINPNNGDTNYAQKFQG | |
| | | (SEQ ID NO: 245)(Kabat) | |
| | | CDR3: VSYYGLDV (SEQ ID | |
| | | NO: 221) | |
| 63. | 1079_A10- | EVQLVESGAEVKKPGASVKV | DIRMTQSPSSLSASVGDRVTI |
| | CEACAM5_ | SCKASGYTFTGYYLHWVRQA | ICRASQSISSYLNWYQQKPG |
| | VH | PGQGLEWMGWINPNNGDTN | KAPKLLIYAASSLQSGVPSRF |
| | Variant | YAQKFQGRVTMTRDTSISTA | SGSGSGTDFTLTISSLQPEDF |
| | | YMELSRLRSDDTAVYYCARV | ATYYCQQSYSNPLTFGGGT |
| | | SYYGLDVWGQGTTVTVSS | KVEIK (SEQ ID NO: 246) |
| | | (SEQ ID NO: 247) | CDR1: RASQSISSYLN (SEQ |
| | | CDR1: GYTFTGY (SEQ ID | ID NO: 223) |
| | | NO: 217)(Chothia) or GYYLH | CDR2: AASSLQS (SEQ ID |
| | | (SEQ ID NO: 228)(Kabat) | NO: 224) |
| | | CDR2: NPNNGD (SEQ ID | CDR3: QQSYSNPLT (SEQ ID |
| | | NO: 244)(Chothia) or | NO: 238) |
| | | WINPNNGDTNYAQKFQG | |
| | | (SEQ ID NO: 245)(Kabat) | |
| | | CDR3: VSYYGLDV (SEQ ID | |
| | | NO: 221) | |
| 64. | 1079_A10- | XVQLVESGAEVKKPGASVKV | DIRMTQSPSSLSASVGDRVTI |
| | CEACAM5 | SCKASGYTFTGYYLHWVRQA | ICRASQSISSYLNWYQQKPG |
| | Consensus | PGQGLEWMGWINPNNGDTN | KAPKLLIYAASSLQSGVPSRF |
| | Sequence | YAQKFQGRVTMTRDTSISTA | SGSGSGTDFTLTISSLQPEDF |
| | | YMELSRLRSDDTAVYYCARV | ATYYCQQSYSNPLTFGGGT |
| | | SYYGLDVWGQGTTVTVSS | KVEIK (SEQ ID NO: 246) |
| | | where X is Q or E | CDR1: RASQSISSYLN (SEQ |
| | | (SEQ ID NO: 248) | ID NO: 223) |
| | | CDR1: GYTFTGY (SEQ ID | CDR2: AASSLQS (SEQ ID |
| | | NO: 217)(Chothia) or GYYLH | NO: 224) |
| | | (SEQ ID NO: 228)(Kabat) | CDR3: QQSYSNPLT (SEQ ID |
| | | CDR2: NPNNGD (SEQ ID | NO: 238) |
| | | NO: 244)(Chothia) or | |
| | | WINPNNGDTNYAQKFQG | |
| | | (SEQ ID NO: 245)(Kabat) | |
| | | CDR3: VSYYGLDV (SEQ ID | |
| | | NO: 221) | |
| 65. | 1079_A12- | QVQLVQSGGGLVQPGGSLRL | DIVLTQTPSSLSASVGDRVTI |
| | CEACAM5 | SCAASGFTFSSYAMNWVRQA | TCRASQSISSWLAWYQQKP |
| | | PGKGLEWVSGISGSGGSTKY | GKAPKLLIYKASSLESGVPS |
| | | AESVKGRFTISRDNSKNTLYL | RFSGSGSGTEFTLTISSLQPD |
| | | QMNSLRADDTAVYYCAKDQ | DFATYYCQQYNSYSPLTFGG |
| | | DINVWYGLFPYWGQGTLVTV | GTKVDIK (SEQ ID NO: 255) |
| | | SS (SEQ ID NO: 249) | CDR1: RASQSISSWLA (SEQ |
| | | CDR1: GFTFSSY (SEQ ID | ID NO: 165) |
| | | NO: 2)(Chothia) or SYAMN | CDR2: KASSLES (SEQ ID |
| | | (SEQ ID NO: 250) | NO: 8) |
| | | (Kabat) | CDR3: QQYNSYSPLT (SEQ |
| | | CDR2: SGSGGS (SEQ ID | ID NO: 256) |
| | | NO: 251)(Chothia) or | |
| | | GISGSGGSTKYAESVKG (SEQ | |
| | | ID NO: 253)(Kabat) | |
| | | CDR3: DQDINVWYGLFPY | |
| | | (SEQ ID NO: 254) | |
| 66. | 1079_A12- | EVQLVQSGGGLVQPGGSLRL | DIVLTQTPSSLSASVGDRVTI |
| | CEACAM5_ | SCAASGFTFSSYAMNWVRQA | TCRASQSISSWLAWYQQKP |
| | VH | PGKGLEWVSGISGSGGSTKY | GKAPKLLIYKASSLESGVPS |
| | Variant | AESVKGRFTISRDNSKNTLYL | RFSGSGSGTEFTLTISSLQPD |
| | | QMNSLRADDTAVYYCAKDQ | DFATYYCQQYNSYSPLTFGG |
| | | DINVWYGLFPYWGQGTLVTV | GTKVDIK (SEQ ID NO: 255) |
| | | SS (SEQ ID NO: 257) | CDR1: RASQSISSWLA (SEQ |
| | | CDR1: GFTFSSY (SEQ ID | ID NO: 165) |
| | | NO: 2)(Chothia) or SYAMN | CDR2: KASSLES (SEQ ID |
| | | (SEQ ID NO: 250)(Kabat) | NO: 8) |
| | | CDR2: SGSGGS (SEQ ID | CDR3: QQYNSYSPLT (SEQ |
| | | NO: 251)(Chothia) or | ID NO: 256) |
| | | GISGSGGSTKYAESVKG (SEQ | |
| | | ID NO: 253)(Kabat) | |
| | | CDR3: DQDINVWYGLFPY | |
| | | (SEQ ID NO: 254) | |
| 67. | 1079_A12- | XVQLVQSGGGLVQPGGSLRL | DIVLTQTPSSLSASVGDRVTI |
| | CEACAM5 | SCAASGFTFSSYAMNWVRQA | TCRASQSISSWLAWYQQKP |
| | Consensus | PGKGLEWVSGISGSGGSTKY | GKAPKLLIYKASSLESGVPS |
| | Sequence | AESVKGRFTISRDNSKNTLYL | RFSGSGSGTEFTLTISSLQPD |
| | | QMNSLRADDTAVYYCAKDQ | DFATYYCQQYNSYSPLTFGG |
| | | DINVWYGLFPYWGQGTLVTV | GTKVDIK (SEQ ID NO: 255) |
| | | SS | CDR1: RASQSISSWLA (SEQ |
| | | where X is Q or E | ID NO: 165) |
| | | (SEQ ID NO: 258) | CDR2: KASSLES (SEQ ID |
| | | CDR1: GFTFSSY (SEQ ID | NO: 8) |
| | | NO: 2)(Chothia) or SYAMN | CDR3: QQYNSYSPLT (SEQ |
| | | (SEQ ID NO: 250)(Kabat) | ID NO: 256) |
| | | CDR2: SGSGGS (SEQ ID | |
| | | NO: 251)(Chothia) or | |
| | | GISGSGGSTKYAESVKG (SEQ | |
| | | ID NO: 253)(Kabat) | |
| | | CDR3: DQDINVWYGLFPY | |
| | | (SEQ ID NO: 254) | |
| 68. | 1078_C04- | EVQLVESGGGVVQPGRSLRL | EIVMTQSPSTLSASVGDRVTI |
| | CEACAM5 | SCAASGFTFSSSGMHWVRQA | TCRASQSISSWLAWYQQKP |
| | | PGKGLEWVAVIWYDGSNKY | GKAPKLLIYKASSLESGVPS |
| | | YVDSVKGRFTISRDNSKNTLY | RFSGSGSGTDFTLTISSLQPD |
| | | LQMNSLRAEDTAVYYCARD | DFATYYCQQYKSYSYTFGQ |
| | | GRQQLVQGSYYYGMDVWG | GTKVEIK (SEQ ID NO: 263) |
| | | QGTTVTVSS (SEQ ID NO: 259) | CDR1: RASQSISSWLA (SEQ |
| | | CDR1: GFTFSSS (SEQ ID | ID NO: 165) |
| | | NO: 260)(Chothia) or SSGMH | CDR2: KASSLES (SEQ ID |
| | | (SEQ ID NO: 261)(Kabat) | NO: 8) |
| | | CDR2: WYDGSN (SEQ ID | CDR3: QQYKSYSYT (SEQ ID |
| | | NO: 70)(Chothia) or | NO: 264) |
| | | VIWYDGSNKYYVDSVKG | |
| | | (SEQ ID NO: 262)(Kabat) | |
| | | CDR3: | |
| | | DGRQQLVQGSYYYGMDV | |
| | | (SEQ ID NO: 265) | |
| 69. | 1080_F11- | QVQLVQSGGGLVQRGGSLRL | DIVMTQTPSTLSASVGDRVT |
| | CEACAM5 | SCAASGFTFSSYAMSWVRQA | ITCRASQSISSWLAWYQQKP |
| | | PGKGLERVSGISGSGGNTYYA | GKAPKLLIYKASSLESGVPS |
| | | DSVKGRFTISRDNSKNTLYLQ | RFSGSGSGTEFTLTISSLQPD |
| | | MNSLRADDTAVYYCARDQDI | DFATYYCQQYNSYFMYTFG |
| | | NVWYGLFAYWGQGTLVTVS | QGTKVEIK (SEQ ID NO: 270) |
| | | S (SEQ ID NO: 266) | CDR1: RASQSISSWLA (SEQ |
| | | CDR1: GFTFSSY (SEQ ID | ID NO: 165) |
| | | NO: 2)(Chothia) or SYAMS | CDR2: KASSLES (SEQ ID |
| | | (SEQ ID NO: 3)(Kabat) | NO: 8) |
| | | CDR2: SGSGGN (SEQ ID | CDR3: QQYNSYFMYT (SEQ |
| | | NO: 267)(Chothia) or | ID NO: 271) |
| | | GISGSGGNTYYADSVKG | |
| | | (SEQ ID NO: 268)(Kabat) | |
| | | CDR3: DQDINVWYGLFAY | |
| | | (SEQ ID NO: 269) | |
| 70. | 1080_F11- | EVQLVQSGGGLVQRGGSLRL | DIVMTQTPSTLSASVGDRVT |
| | CEACAM5_ | SCAASGFTFSSYAMSWVRQA | ITCRASQSISSWLAWYQQKP |
| | VH | PGKGLERVSGISGSGGNTYYA | GKAPKLLIYKASSLESGVPS |
| | Variant | DSVKGRFTISRDNSKNTLYLQ | RFSGSGSGTEFTLTISSLQPD |
| | | MNSLRADDTAVYYCARDQDI | DFATYYCQQYNSYFMYTFG |
| | | NVWYGLFAYWGQGTLVTVS | QGTKVEIK (SEQ ID NO: 270) |
| | | S (SEQ ID NO: 272) | CDR1: RASQSISSWLA (SEQ |
| | | CDR1: GFTFSSY (SEQ ID | ID NO: 165) |
| | | NO: 2)(Chothia) or SYAMS | CDR2: KASSLES (SEQ ID |
| | | (SEQ ID NO: 3)(Kabat) | NO: 8) |
| | | CDR2: SGSGGN (SEQ ID | CDR3: QQYNSYFMYT (SEQ |
| | | NO: 267)(Chothia) or | ID NO: 271) |
| | | GISGSGGNTYYADSVKG | |
| | | (SEQ ID NO: 268)(Kabat) | |
| | | CDR3: DQDINVWYGLFAY | |
| | | (SEQ ID NO: 269) | |
| 71. | 1080_F11- | XVQLVQSGGGLVQRGGSLRL | DIVMTQTPSTLSASVGDRVT |
| | CEACAM5 | SCAASGFTFSSYAMSWVRQA | ITCRASQSISSWLAWYQQKP |
| | Consensus | PGKGLERVSGISGSGGNTYYA | GKAPKLLIYKASSLESGVPS |
| | Sequence | DSVKGRFTISRDNSKNTLYLQ | RFSGSGSGTEFTLTISSLQPD |
| | | MNSLRADDTAVYYCARDQDI | DFATYYCQQYNSYFMYTFG |
| | | NVWYGLFAYWGQGTLVTVS | QGTKVEIK (SEQ ID NO: 270) |
| | | S | CDR1: RASQSISSWLA (SEQ |
| | | where X is Q or E | ID NO: 165) |
| | | (SEQ ID NO: 273) | CDR2: KASSLES (SEQ ID |
| | | CDR1: GFTFSSY (SEQ ID | NO: 8) |
| | | NO: 2)(Chothia) or SYAMS | CDR3: QQYNSYFMYT (SEQ |
| | | (SEQ ID NO: 3)(Kabat) | ID NO: 271) |
| | | CDR2: SGSGGN (SEQ ID | |
| | | NO: 267)(Chothia) or | |
| | | GISGSGGNTYYADSVKG | |
| | | (SEQ ID NO: 268)(Kabat) | |
| | | CDR3: DQDINVWYGLFAY | |
| | | (SEQ ID NO: 269) | |
| 72. | 1081_E01- | QVQLQESGPGLVKPSETLSLT | DIVMTQTPSTLSASVGDRVT |
| | CEACAM5 | CAVSGYSISSGYYWGWIRQPP | ITCRASQSISSWLAWYQQKP |
| | | GKGLEWIGTIWHSGSTYDNPS | GKAPKLLIYKASSLESGVPS |
| | | LKSRVTISVDTSKNQFSLKLSS | RFSGSGSGTDFTLTISSLQPE |
| | | VTTADTAVYYCARADSSGW | DFATYYCQQSYSTPLTFGGG |
| | | YEDYWGQGTLVTVSS (SEQ | TKVEIK (SEQ ID NO: 280) |
| | | ID NO: 274) | CDR1: RASQSISSWLA (SEQ |
| | | CDR1: GYSISSGY (SEQ ID | ID NO: 165) |
| | | NO: 275)(Chothia) or SGYYWG | CDR2: KASSLES (SEQ ID |
| | | (SEQ ID NO: 276)(Kabat) | NO: 8) |
| | | CDR2: WHSGS (SEQ ID | CDR3: QQSYSTPLT (SEQ ID |
| | | NO: 277)(Chothia) or | NO: 233) |
| | | TIWHSGSTYDNPSLKS (SEQ | |
| | | ID NO: 278)(Kabat) | |
| | | CDR3: ADSSGWYEDY (SEQ | |
| | | ID NO: 279) | |
| 73. | 1081_E01- | EVQLQESGPGLVKPSETLSLT | DIVMTQTPSTLSASVGDRVT |
| | CEACAM5_ | CAVSGYSISSGYYWGWIRQPP | ITCRASQSISSWLAWYQQKP |
| | VH | GKGLEWIGTIWHSGSTYDNPS | GKAPKLLIYKASSLESGVPS |
| | Variant | LKSRVTISVDTSKNQFSLKLSS | RFSGSGSGTDFTLTISSLQPE |
| | | VTTADTAVYYCARADSSGW | DFATYYCQQSYSTPLTFGGG |
| | | YEDYWGQGTLVTVSS (SEQ | TKVEIK (SEQ ID NO: 280) |
| | | ID NO: 281) | CDR1: RASQSISSWLA (SEQ |
| | | CDR1: GYSISSGY (SEQ ID | ID NO: 165) |
| | | NO: 275)(Chothia) or SGYYWG | CDR2: KASSLES (SEQ ID |
| | | (SEQ ID NO: 276)(Kabat) | NO: 8) |
| | | CDR2: WHSGS (SEQ ID | CDR3: QQSYSTPLT (SEQ ID |
| | | NO: 277)(Chothia) or | NO: 233) |
| | | TIWHSGSTYDNPSLKS (SEQ | |
| | | ID NO: 278)(Kabat) | |
| | | CDR3: ADSSGWYEDY (SEQ | |
| | | ID NO: 279) | |
| 74. | 1081_E01- | XVQLQESGPGLVKPSETLSLT | DIVMTQTPSTLSASVGDRVT |
| | CEACAM5 | CAVSGYSISSGYYWGWIRQPP | ITCRASQSISSWLAWYQQKP |
| | Consensus | GKGLEWIGTIWHSGSTYDNPS | GKAPKLLIYKASSLESGVPS |
| | Sequence | LKSRVTISVDTSKNQFSLKLSS | RFSGSGSGTDFTLTISSLQPE |
| | | VTTADTAVYYCARADSSGW | DFATYYCQQSYSTPLTFGGG |
| | | YEDYWGQGTLVTVSS | TKVEIK (SEQ ID NO: 280) |
| | | where X is Q or E | CDR1: RASQSISSWLA (SEQ |
| | | (SEQ ID NO: 282) | ID NO: 165) |
| | | CDR1: GYSISSGY (SEQ ID | CDR2: KASSLES (SEQ ID |
| | | NO: 275)(Chothia) or SGYYWG | NO: 8) |
| | | (SEQ ID NO: 276)(Kabat) | CDR3: QQSYSTPLT (SEQ ID |
| | | CDR2: WHSGS (SEQ ID | NO: 233) |
| | | NO: 277)(Chothia) or | |
| | | TIWHSGSTYDNPSLKS (SEQ | |
| | | ID NO: 278)(Kabat) | |
| | | CDR3: ADSSGWYEDY (SEQ | |
| | | ID NO: 279) | |
| 75. | 1083_A05- | QVQLVQSGGGLVQPGGSLRL | DIQVTQSPSTLSASVGDRVTI |
| | CEACAM5 | SCAASGFIFSSYAMSWVRQA | TCRASQSISSWLAWYQQKP |
| | | PGRGLEWVSGISGSGGSTNY | GKAPKLLIYKASSLESGVPS |
| | | ADSVKGRFTISRDNSKNTLYL | RFSGSGSGTEFTLTISSLQPD |
| | | QMNSLRADDTAVYFCAKDL | DFATYYCQQYNSYSTWTFG |
| | | DINVWYGLFDHWGQGTTVT | QGTKLEIK (SEQ ID NO: 286) |
| | | VSS (SEQ ID NO: 283) | CDR1: RASQSISSWLA (SEQ |
| | | CDR1: GFIFSSY (SEQ ID | ID NO: 165) |
| | | NO: 284)(Chothia) or SYAMS | CDR2: KASSLES (SEQ ID |
| | | (SEQ ID NO: 3)(Kabat) | NO: 8) |
| | | CDR2: SGSGGS (SEQ ID | CDR3: QQYNSYSTWT (SEQ |
| | | NO: 251)(Chothia) or | ID NO: 166) |
| | | GISGSGGSTNYADSVKG (SEQ | |
| | | ID NO: 252)(Kabat) | |
| | | CDR3: DLDINVWYGLFDH | |
| | | (SEQ ID NO: 285) | |
| 76. | 1083_A05- | EVQLVQSGGGLVQPGGSLRL | DIQVTQSPSTLSASVGDRVTI |
| | CEACAM5_ | SCAASGFIFSSYAMSWVRQA | TCRASQSISSWLAWYQQKP |
| | VH | PGRGLEWVSGISGSGGSTNY | GKAPKLLIYKASSLESGVPS |
| | Variant | ADSVKGRFTISRDNSKNTLYL | RFSGSGSGTEFTLTISSLQPD |
| | | QMNSLRADDTAVYFCAKDL | DFATYYCQQYNSYSTWTFG |
| | | DINVWYGLFDHWGQGTTVT | QGTKLEIK (SEQ ID NO: 286) |
| | | VSS (SEQ ID NO: 287) | CDR1: RASQSISSWLA (SEQ |
| | | CDR1: GFIFSSY (SEQ ID | ID NO: 165) |
| | | NO: 284)(Chothia) or SYAMS | CDR2: KASSLES (SEQ ID |
| | | (SEQ ID NO: 3)(Kabat) | NO: 8) |
| | | CDR2: SGSGGS (SEQ ID | CDR3: QQYNSYSTWT (SEQ |
| | | NO: 251)(Chothia) or | ID NO: 166) |
| | | GISGSGGSTNYADSVKG (SEQ | |
| | | ID NO: 252)(Kabat) | |
| | | CDR3: DLDINVWYGLFDH | |
| | | (SEQ ID NO: 285) | |
| 77. | 1083_A05- | XVQLVQSGGGLVQPGGSLRL | DIQVTQSPSTLSASVGDRVTI |
| | CEACAM5 | SCAASGFIFSSYAMSWVRQA | TCRASQSISSWLAWYQQKP |
| | Consensus | PGRGLEWVSGISGSGGSTNY | GKAPKLLIYKASSLESGVPS |
| | Sequence | ADSVKGRFTISRDNSKNTLYL | RFSGSGSGTEFTLTISSLQPD |
| | | QMNSLRADDTAVYFCAKDL | DFATYYCQQYNSYSTWTFG |
| | | DINVWYGLFDHWGQGTTVT | QGTKLEIK (SEQ ID NO: 286) |
| | | VSS | CDR1: RASQSISSWLA (SEQ |
| | | where X is Q or E | ID NO: 165) |
| | | (SEQ ID NO: 289) | CDR2: KASSLES (SEQ ID |
| | | CDR1: GFIFSSY (SEQ ID | NO: 8) |
| | | NO: 284)(Chothia) or SYAMS | CDR3: QQYNSYSTWT (SEQ |
| | | (SEQ ID NO: 3)(Kabat) | ID NO: 166) |
| | | CDR2: SGSGGS (SEQ ID | |
| | | NO: 251)(Chothia) or | |
| | | GISGSGGSTNYADSVKG (SEQ | |
| | | ID NO: 252)(Kabat) | |
| | | CDR3: DLDINVWYGLFDH | |
| | | (SEQ ID NO: 285) | |
| 78. | 1085_D12- | EVQLVESGGGLVQPGGSLRL | DIRLTQSPSTLSASVGDRVTI |
| | CEACAM5 | SCAASGFIFSSYAMNWVRQA | TCRASQSISSWLAWYQQKP |
| | | PGRGLEWVSGISGSGGSTNY | GKAPKLLIYKASSLESGVPS |
| | | ADSVKGRFTISRDNSKNTLYL | RFSGSGSGTEFTLTISSMQPD |
| | | QMNSLRADDTAVYFCARDQ | DFATYYCQQYNSYSTWTFG |
| | | DINVWYGLFVYWGQGTLVT | QGTKVEIK (SEQ ID NO: 291) |
| | | VSS (SEQ ID NO: 288) | CDR1: RASQSISSWLA (SEQ |
| | | CDR1: GFIFSSY (SEQ ID | ID NO: 165) |
| | | NO: 284)(Chothia) or SYAMN | CDR2: KASSLES (SEQ ID |
| | | (SEQ ID NO: 250)(Kabat) | NO: 8) |
| | | CDR2: SGSGGS (SEQ ID | CDR3: QQYNSYSTWT (SEQ |
| | | NO: 251)(Chothia) or | ID NO: 166) |
| | | GISGSGGSTNYADSVKG (SEQ | |
| | | ID NO: 252)(Kabat) | |
| | | CDR3: DQDINVWYGLFVY | |
| | | (SEQ ID NO: 290) | |
| 79. | 1079_G12- | QVQLVQSGGGLVQPGGSLRL | DIVMTQTPSSLSASVGDRVTI |
| | CEACAM5 | SCAASGFTFSSYAMNWVRQA | TCRASQGISNYLAWYQQKP |
| | | PGKGLEWVSVISGNGGYTHY | GKVPKLLIYAASTLQSGVPS |
| | | ADSVKGRFTISRDNSKNTLYL | RFSGSGSGTDFTLTISSLQPE |
| | | QMNSLRAEGTAVYYCAKDD | DVATYYCQKYNSAPRTFGQ |
| | | WGGAFDIWGQGTTVTVSS | GTKVEIK (SEQ ID NO: 296) |
| | | (SEQ ID NO: 292) | CDR1: RASQGISNYLA (SEQ |
| | | CDR1: GFTFSSY (SEQ ID | ID NO: 127) |
| | | NO: 2)(Chothia) or SYAMN | CDR2: AASTLQS (SEQ ID |
| | | (SEQ ID NO: 250)(Kabat) | NO: 128) |
| | | CDR2: SGNGGY (SEQ ID | CDR3: QKYNSAPRT (SEQ ID |
| | | NO: 293)(Chothia) or | NO: 297) |
| | | VISGNGGYTHYADSVKG | |
| | | (SEQ ID NO: 294)(Kabat) | |
| | | CDR3: DDWGGAFDI (SEQ ID | |
| | | NO: 295) | |
| 80. | 1079_G12- | EVQLVQSGGGLVQPGGSLRL | DIVMTQTPSSLSASVGDRVTI |
| | CEACAM5_ | SCAASGFTFSSYAMNWVRQA | TCRASQGISNYLAWYQQKP |
| | VH | PGKGLEWVSVISGNGGYTHY | GKVPKLLIYAASTLQSGVPS |
| | Variant | ADSVKGRFTISRDNSKNTLYL | RFSGSGSGTDFTLTISSLQPE |
| | | QMNSLRAEGTAVYYCAKDD | DVATYYCQKYNSAPRTFGQ |
| | | WGGAFDIWGQGTTVTVSS | GTKVEIK (SEQ ID NO: 296) |
| | | (SEQ ID NO: 298) | CDR1: RASQGISNYLA (SEQ |
| | | CDR1: GFTFSSY (SEQ ID | ID NO: 127) |
| | | NO: 2)(Chothia) or SYAMN | CDR2: AASTLQS (SEQ ID |
| | | (SEQ ID NO: 250)(Kabat) | NO: 128) |
| | | CDR2: SGNGGY (SEQ ID | CDR3: QKYNSAPRT (SEQ ID |
| | | NO: 293)(Chothia) or | NO: 297) |
| | | VISGNGGYTHY ADSVKG | |
| | | (SEQ ID NO: 294)(Kabat) | |
| | | CDR3: DDWGGAFDI (SEQ ID | |
| | | NO: 295) | |
| 81. | 1080_A01- | EVQLVQSGGGLVQPGGSVRL | DIRMTQSPSSLSASVGDRVTI |
| | CEACAM5 | SCAASGFTFSSYAMNWVRQG | TCRASQGISNYLAWYQQKP |
| | | PGKGLEWVSVISGNGGYTHY | GKVPKLLIYAASTLQSGVPS |
| | | ADSVKGRFTISRDNSKNTLYL | RFSGSGSGTDFTLTISSLQPE |
| | | QMNGLRADDTAVYYCAKDD | DVATYYCQKYNSAPRTFGQ |
| | | WGGAFDIWGQGTMVTVSS | GTKVDIK (SEQ ID NO: 300) |
| | | (SEQ ID NO: 299) | CDR1: RASQGISNYLA (SEQ |
| | | CDR1: GFTFSSY (SEQ ID | ID NO: 127) |
| | | NO: 2)(Chothia) or SYAMN | CDR2: AASTLQS (SEQ ID |
| | | (SEQ ID NO: 250)(Kabat) | NO: 128) |
| | | CDR2: SGNGGY (SEQ ID | CDR3: QKYNSAPRT (SEQ ID |
| | | NO: 293)(Chothia) or | NO: 297) |
| | | VISGNGGYTHY ADSVKG | |
| | | (SEQ ID NO: 294)(Kabat) | |
| | | CDR3: DDWGGAFDI (SEQ ID | |
| | | NO: 295) | |
| 82. | VH | XVQLVQSGGGLVQPGGSLRL | DIX₁MTQX₂PSSLSASVGDRV |
| | [1079_G12- | SCAASGFTFSSYAMNWVRQA | TITCRASQGISNYLAWYQQK |
| | CEACAM] | PGKGLEWVSVISGNGGYTHY | PGKVPKLLIYAASTLQSGVP |
| | and | ADSVKGRFTISRDNSKNTLYL | SRFSGSGSGTDFTLTISSLQP |
| | VL | QMNSLRAEGTAVYYCAKDD | EDVATYYCQKYNSAPRTFG |
| | Consensus | WGGAFDIWGQGTTVTVSS | QGTKVX₃IK |
| | Sequence | where X is Q or E | where X₁ is V or R, X₂ is S or T, |
| | [1079_G12- | (SEQ ID NO: 301) | and X₃ is D or E |
| | CEACAM; | CDR1: GFTFSSY (SEQ ID | (SEQ ID NO: 302) |
| | 1080_A01- | NO: 2)(Chothia) or SYAMN | CDR1: RASQGISNYLA (SEQ |
| | CEACAM5] | (SEQ ID NO: 250)(Kabat) | ID NO: 127) |
| | | CDR2: SGNGGY (SEQ ID | CDR2: AASTLQS (SEQ ID |
| | | NO: 293)(Chothia) or | NO: 128) |
| | | VISGNGGYTHYADSVKG | CDR3: QKYNSAPRT (SEQ ID |
| | | (SEQ ID NO: 294)(Kabat) | NO: 297) |
| | | CDR3: DDWGGAFDI (SEQ ID | |
| | | NO: 295) | |
| 83. | VH | EVQLVQSGGGLVQPGGSVRL | DIX₁MTQX₂PSSLSASVGDRV |
| | [1080_A01- | SCAASGFTFSSYAMNWVRQG | TITCRASQGISNYLAWYQQK |
| | CEACAM5] | PGKGLEWVSVISGNGGYTHY | PGKVPKLLIYAASTLQSGVP |
| | and | ADSVKGRFTISRDNSKNTLYL | SRFSGSGSGTDFTLTISSLQP |
| | VL | QMNGLRADDTAVYYCAKDD | EDVATYYCQKYNSAPRTFG |
| | Consensus | WGGAFDIWGQGTMVTVSS | QGTKVX₃IK |
| | Sequence | (SEQ ID NO: 299) | where X₁ is V or R, X₂ is S or T, |
| | [1079_G12- | CDR1: GFTFSSY (SEQ ID | and X₃ is D or E |
| | CEACAM; | NO: 2)(Chothia) or SYAMN | (SEQ ID NO: 302) |
| | 1080_A01- | (SEQ ID NO: 250)(Kabat) | CDR1: RASQGISNYLA (SEQ |
| | CEACAM5] | CDR2: SGNGGY (SEQ ID | ID NO: 127) |
| | | NO: 293)(Chothia) or | CDR2: AASTLQS (SEQ ID |
| | | VISGNGGYTHYADSVKG | NO: 128) |
| | | (SEQ ID NO: 294)(Kabat) | CDR3: QKYNSAPRT (SEQ ID |
| | | CDR3: DDWGGAFDI (SEQ ID | NO: 297) |
| | | NO: 295) | |
| 84. | 12C7.A2- | QVQLVESGGGVVQPGRSLRL | DIQLTQSPSFLSASVGDRVTI |
| | CEACAM5- | SCAASGFTFSNYAMHWVRQ | TCRASQGISSYLAWYQQKP |
| | B.01 | APGKGLEWVTLIWYDGSKKY | GKAPKLLIFPASTLQSGVPSR |
| | | YADSVKGRFTISRDNSKNTLY | FSGSGSGTEFTLTISSLQPEDF |
| | | LQMNSLRAEDTAVYYCARD | ATYYCQQLNFFPPTFGPGTK |
| | | RAIPLIAPYFDYWGQGTLVTV | VDIK (SEQ ID NO: 309) |
| | | SS (SEQ ID NO: 303) | CDR1: RASQGISSYLA (SEQ |
| | | CDR1: GFTFSNY (SEQ ID | ID NO: 131) |
| | | NO: 304)(Chothia) or NYAMH | CDR2: PASTLQS (SEQ ID |
| | | (SEQ ID NO: 305)(Kabat) | NO: 310) |
| | | CDR2: WYDGSK (SEQ ID | CDR3: QQLNFFPPT (SEQ ID |
| | | NO: 306)(Chothia) or | NO: 311) |
| | | LIWYDGSKKYYADSVKG | |
| | | (SEQ ID NO: 307)(Kabat) | |
| | | CDR3: DRAIPLIAPYFDY (SEQ | |
| | | ID NO: 308) | |
| 85. | 12C7.A2- | EVQLVESGGGVVQPGRSLRL | DIQLTQSPSFLSASVGDRVTI |
| | CEACAM5- | SCAASGFTFSNYAMHWVRQ | TCRASQGISSYLAWYQQKP |
| | B.01_VH | APGKGLEWVTLIWYDGSKKY | GKAPKLLIFPASTLQSGVPSR |
| | Variant | YADSVKGRFTISRDNSKNTLY | FSGSGSGTEFTLTISSLQPEDF |
| | | LQMNSLRAEDTAVYYCARD | ATYYCQQLNFFPPTFGPGTK |
| | | RAIPLIAPYFDYWGQGTLVTV | VDIK (SEQ ID NO: 309) |
| | | SS (SEQ ID NO: 312) | CDR1: RASQGISSYLA (SEQ |
| | | CDR1: GFTFSNY (SEQ ID | ID NO: 131) |
| | | NO: 304)(Chothia) or NYAMH | CDR2: PASTLQS (SEQ ID |
| | | (SEQ ID NO: 305)(Kabat) | NO: 310) |
| | | CDR2: WYDGSK (SEQ ID | CDR3: QQLNFFPPT (SEQ ID |
| | | NO: 306)(Chothia) or | NO: 311) |
| | | LIWYDGSKKYYADSVKG | |
| | | (SEQ ID NO: 307)(Kabat) | |
| | | CDR3: DRAIPLIAPYFDY (SEQ | |
| | | ID NO: 308) | |
| 86. | 12C7.A2- | XVQLVESGGGVVQPGRSLRL | DIQLTQSPSFLSASVGDRVTI |
| | CEACAM5- | SCAASGFTFSNYAMHWVRQ | TCRASQGISSYLAWYQQKP |
| | B.01 | APGKGLEWVTLIWYDGSKKY | GKAPKLLIFPASTLQSGVPSR |
| | Consensus | YADSVKGRFTISRDNSKNTLY | FSGSGSGTEFTLTISSLQPEDF |
| | Sequence | LQMNSLRAEDTAVYYCARD | ATYYCQQLNFFPPTFGPGTK |
| | | RAIPLIAPYFDYWGQGTLVTV | VDIK (SEQ ID NO: 309) |
| | | SS | CDR1: RASQGISSYLA (SEQ |
| | | where X is Q or E | ID NO: 131) |
| | | (SEQ ID NO: 313) | CDR2: PASTLQS (SEQ ID |
| | | CDR1: GFTFSNY (SEQ ID | NO: 310) |
| | | NO: 304)(Chothia) or NYAMH | CDR3: QQLNFFPPT (SEQ ID |
| | | (SEQ ID NO: 305)(Kabat) | NO: 311) |
| | | CDR2: WYDGSK (SEQ ID | |
| | | NO: 306)(Chothia) or | |
| | | LIWYDGSKKYYADSVKG | |
| | | (SEQ ID NO: 307)(Kabat) | |
| | | CDR3: DRAIPLIAPYFDY (SEQ | |
| | | ID NO: 308) | |
| 87. | 12C7.A2- | QVQLQESGPGLVKPSETLSLT | DIQLTQSPSFLSASVGDRVTI |
| | CEACAM5- | CTVSGGPIYSYYWSWIRQPPG | TCRASQGISSYLAWYQQKP |
| | B.01_VH2 | KGLEWIGFIYSSGSTNYNPSL | GKAPKLLIFPASTLQSGVPSR |
| | | KSRVTISIDTSKSQFSLRLSSV | FSGSGSGTEFTLTISSLQPEDF |
| | | TAADTAVYYCARGGDAFDI | ATYYCQQLNFFPPTFGPGTK |
| | | WGQGTMVTVSS (SEQ ID | VDIK (SEQ ID NO: 309) |
| | | NO: 691) | CDR1: RASQGISSYLA (SEQ |
| | | CDR1: GGPIYSY (SEQ ID | ID NO: 131) |
| | | NO: 315)(Chothia) or SYYWS | CDR2: PASTLQS (SEQ ID |
| | | (SEQ ID NO: 316)(Kabat) | NO: 310) |
| | | CDR2: YSSGS (SEQ ID | CDR3: QQLNFFPPT (SEQ ID |
| | | NO: 317)(Chothia) or | NO: 311) |
| | | FIYSSGSTNYNPSLKS (SEQ ID | |
| | | NO: 318)(Kabat) | |
| | | CDR3: GGDAFDI (SEQ ID | |
| | | NO: 319) | |
| 88. | 12A6.H2- | QVQLVESGPGLVKPSETLSLT | DIQLTQSPSFLSASVGDRVTI |
| | CEACAM5- | CTVSGGPIYSYYWSWIRQPPG | TCRASQGISSYLAWYQQKP |
| | B.01 | KGLEWIGFIYSSGSTNYNPSL | GKAPKLLIFPASTLQSGVPSR |
| | | KSRVTISIDTSKSQFSLRLSSV | FSGSGSGTEFTLTISSLQPEDF |
| | | TAADTAVYYCARGGDAFDI | ATYYCQQLNFFPPTFGPGTK |
| | | WGQGTMVTVSS (SEQ ID | VDIK (SEQ ID NO: 309) |
| | | NO: 314) | CDR1: RASQGISSYLA (SEQ |
| | | CDR1: GGPIYSY (SEQ ID | ID NO: 131) |
| | | NO: 315)(Chothia) or SYYWS | CDR2: PASTLQS (SEQ ID |
| | | (SEQ ID NO: 316)(Kabat) | NO: 310) |
| | | CDR2: YSSGS (SEQ ID | CDR3: QQLNFFPPT (SEQ ID |
| | | NO: 317)(Chothia) or | NO: 311) |
| | | FIYSSGSTNYNPSLKS (SEQ ID | |
| | | NO: 318)(Kabat) | |
| | | CDR3: GGDAFDI (SEQ ID | |
| | | NO: 319) | |
| 89. | 12A6.H2- | EVQLVESGPGLVKPSETLSLT | DIQLTQSPSFLSASVGDRVTI |
| | CEACAM5- | CTVSGGPIYSYYWSWIRQPPG | TCRASQGISSYLAWYQQKP |
| | B.01_VH | KGLEWIGFIYSSGSTNYNPSL | GKAPKLLIFPASTLQSGVPSR |
| | Variant | KSRVTISIDTSKSQFSLRLSSV | FSGSGSGTEFTLTISSLQPEDF |
| | | TAADTAVYYCARGGDAFDI | ATYYCQQLNFFPPTFGPGTK |
| | | WGQGTMVTVSS (SEQ ID | VDIK (SEQ ID NO: 309) |
| | | NO: 320) | CDR1: RASQGISSYLA (SEQ |
| | | CDR1: GGPIYSY (SEQ ID | ID NO: 131) |
| | | NO: 315)(Chothia) or SYYWS | CDR2: PASTLQS (SEQ ID |
| | | (SEQ ID NO: 316)(Kabat) | NO: 310) |
| | | CDR2: YSSGS (SEQ ID | CDR3: QQLNFFPPT (SEQ ID |
| | | NO: 317)(Chothia) or | NO: 311) |
| | | FIYSSGSTNYNPSLKS (SEQ ID | |
| | | NO: 318)(Kabat) | |
| | | CDR3: GGDAFDI (SEQ ID | |
| | | NO: 319) | |
| 90 | 12A6.H2- | XVQLVESGPGLVKPSETLSLT | DIQLTQSPSFLSASVGDRVTI |
| | CEACAM5- | CTVSGGPIYSYYWSWIRQPPG | TCRASQGISSYLAWYQQKP |
| | B.01 | KGLEWIGFIYSSGSTNYNPSL | GKAPKLLIFPASTLQSGVPSR |
| | Consensus | KSRVTISIDTSKSQFSLRLSSV | FSGSGSGTEFTLTISSLQPEDF |
| | Sequence | TAADTAVYYCARGGDAFDI | ATYYCQQLNFFPPTFGPGTK |
| | | WGQGTMVTVSS | VDIK (SEQ ID NO: 309) |
| | | where X is Q or E | CDR1: RASQGISSYLA (SEQ |
| | | (SEQ ID NO: 321) | ID NO: 131) |
| | | CDR1: GGPIYSY (SEQ ID | CDR2: PASTLQS (SEQ ID |
| | | NO: 315)(Chothia) or SYYWS | NO: 310) |
| | | (SEQ ID NO: 316)(Kabat) | CDR3: QQLNFFPPT (SEQ ID |
| | | CDR2: YSSGS (SEQ ID | NO: 311) |
| | | NO: 317)(Chothia) or | |
| | | FIYSSGSTNYNPSLKS (SEQ ID | |
| | | NO: 318)(Kabat) | |
| | | CDR3: GGDAFDI (SEQ ID | |
| | | NO: 319) | |
| 91. | 4G3.C3- | QVQLQESGPGLVKPSETLSLT | DIQLTQSPSFLSASVGDRVTI |
| | CEACAM5- | CTVSGGSLSSYYWSWIRQPPG | TCRASQGTSSYLAWYQQKP |
| | B.01 | KGLEWLGYIYSSGSVNYNPSL | GKAPKLLIYAVSTLQSGVPS |
| | | KSRVTMSIDTSQNQFSLKLSS | RFSGSGSGTEFTLTISSLQPT |
| | | VTAADTAVYYCARDADYFD | DFATYYCQQVIRYPPTFGQG |
| | | YWGQGTLVTVSS (SEQ ID | TKVEVK (SEQ ID NO: 326) |
| | | NO: 322) | CDR1: RASQGTSSYLA (SEQ |
| | | CDR1: GGSLSSY (SEQ ID | ID NO: 327) |
| | | NO: 323)(Chothia) or SYYWS | CDR2: AVSTLQS (SEQ ID |
| | | (SEQ ID NO: 316)(Kabat) | NO: 328) |
| | | CDR2: YSSGS (SEQ ID | CDR3: QQVIRYPPT (SEQ ID |
| | | NO: 317)(Chothia) or | NO: 329) |
| | | YIYSSGSVNYNPSLKS (SEQ | |
| | | ID NO: 324)(Kabat) | |
| | | CDR3: DADYFDY (SEQ ID | |
| | | NO: 325) | |
| 92. | 4G3.C3- | EVQLQESGPGLVKPSETLSLT | DIQLTQSPSFLSASVGDRVTI |
| | CEACAM5- | CTVSGGSLSSYYWSWIRQPPG | TCRASQGTSSYLAWYQQKP |
| | B.01_VH | KGLEWLGYIYSSGSVNYNPSL | GKAPKLLIYAVSTLQSGVPS |
| | Variant | KSRVTMSIDTSQNQFSLKLSS | RFSGSGSGTEFTLTISSLQPT |
| | | VTAADTAVYYCARDADYFD | DFATYYCQQVIRYPPTFGQG |
| | | YWGQGTLVTVSS (SEQ ID | TKVEVK (SEQ ID NO: 326) |
| | | NO: 330) | CDR1: RASQGTSSYLA (SEQ |
| | | CDR1: GGSLSSY (SEQ ID | ID NO: 327) |
| | | NO: 323)(Chothia) or SYYWS | CDR2: AVSTLQS (SEQ ID |
| | | (SEQ ID NO: 316)(Kabat) | NO: 328) |
| | | CDR2: YSSGS (SEQ ID | CDR3: QQVIRYPPT (SEQ ID |
| | | NO: 317)(Chothia) or | NO: 329) |
| | | YIYSSGSVNYNPSLKS (SEQ | |
| | | ID NO: 324)(Kabat) | |
| | | CDR3: DADYFDY (SEQ ID | |
| | | NO: 325) | |
| 93. | 4G3.C3- | XVQLQESGPGLVKPSETLSLT | DIQLTQSPSFLSASVGDRVTI |
| | CEACAM5- | CTVSGGSLSSYYWSWIRQPPG | TCRASQGTSSYLAWYQQKP |
| | B.01 | KGLEWLGYIYSSGSVNYNPSL | GKAPKLLIYAVSTLQSGVPS |
| | Consensus | KSRVTMSIDTSQNQFSLKLSS | RFSGSGSGTEFTLTISSLQPT |
| | Sequence | VTAADTAVYYCARDADYFD | DFATYYCQQVIRYPPTFGQG |
| | | YWGQGTLVTVSS | TKVEVK (SEQ ID NO: 326) |
| | | where X is Q or E | CDR1: RASQGTSSYLA (SEQ |
| | | (SEQ ID NO: 331) | ID NO: 327) |
| | | CDR1: GGSLSSY (SEQ ID | CDR2: AVSTLQS (SEQ ID |
| | | NO: 323)(Chothia) or SYYWS | NO: 328) |
| | | (SEQ ID NO: 316)(Kabat) | CDR3: QQVIRYPPT (SEQ ID |
| | | CDR2: YSSGS (SEQ ID | NO: 329) |
| | | NO: 317)(Chothia) or | |
| | | YIYSSGSVNYNPSLKS (SEQ | |
| | | ID NO: 324)(Kabat) | |
| | | CDR3: DADYFDY (SEQ ID | |
| | | NO: 325) | |
| 94. | 6D10.C8- | QIQLVQSGPELKKPGETVKIS | QIVLTQSPAIMSASPGEKVTL |
| | CEACAM5- | CKASGYTFTNYGMNWVKQA | TCSPSSSVTYMHWYQQKSG |
| | B.02_VH | PGKGLKWMGWINTYTGEPT | TSPKRWIYDTSKLASGVPAR |
| | and | YTDDFKGRFAFSLETSASTAY | FSGIGSGTSYSLTINSMEAED |
| | 6D10.C8- | LQITNLKNEDTATYFCARGD | AATYYCQQWNNYPYTFGG |
| | CEACAM5- | GFDRGFAYWGQGTLVTVSA | GTRLEIT (SEQ ID NO: 698) |
| | B.02_VL | (SEQ ID NO: 692) | CDR1: SPSSSVTYMH (SEQ |
| | | CDR1: GYTFTNY (SEQ ID | ID NO: 658) |
| | | NO: 693)(Chothia) or NYGMN | CDR2: DTSKLAS (SEQ ID |
| | | (SEQ ID NO: 694)(Kabat) | NO: 659) |
| | | CDR2: NTYTGE (SEQ ID | CDR3: QQWNNYPYT (SEQ |
| | | NO: 695)(Chothia) or | ID NO: 660) |
| | | WINTYTGEPTYTDDFKG (SEQ | |
| | | ID NO: 696)(Kabat) | |
| | | CDR3: GDGFDRGFAY (SEQ ID | |
| | | NO: 697) | |

TABLE 4
Sequences of Exemplary Antigen-Binding Sites that Bind CEACAM5

| | | | |
|---|---|---|---|
| Clone | Variant | VH | VL |

Tier 1

| | | | |
|---|---|---|---|
| Clone | Clone | QVQLVQSGGGLVQPGGSLRL | DIRMTQSPSTLSASVGDR |
| PH_420- | PH_420- | SCAASGFTFSSYAMSWVRQA | VTITCWASQSISSWLAWY |
| CEACAM5 | CEACAM5 | PGKGLEWVSAIFNSGGSTYY | QQKPGKAPKLLIYKASSL |
| | | ADSVKGRFTVSRDNSKNTLY | ESGVPSRFSGSGSGTEFTL |
| | | LQMNSLRAEDTALYYCAKDL | TISSLQPDDFATYYCQQY |
| | | GGYNYGLFDYWGQGTLVTV | NSYSYTFGQGTKLEIK |
| | | SS (SEQ ID NO: 719) | (SEQ ID NO: 10) |
| | | CDR1: GFTFSSY (SEQ ID | CDR1: WASQSISSWLA |
| | | NO: 2)(Chothia) or SYAMS | (SEQ ID NO: 7) |
| | | (SEQ ID NO: 3)(Kabat) | CDR2: KASSLES (SEQ ID |
| | | CDR2: FNSGGS (SEQ ID NO: 4) | NO: 8) |
| | | (Chothia) or | CDR3: QQYNSYSYT (SEQ |
| | | AIFNSGGSTYYADSVKG (SEQ | ID NO: 9) |
| | | ID NO: 5)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNSGGSTYY | |
| | Variant 1 (QE) | ADSVKGRFTVSRDNSKNTLY | |
| | | LQMNSLRAEDTALYYCAKDL | |
| | | GGYNYGLFDYWGQGTLVTV | |
| | | SS (SEQ ID NO: 11) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNAGGSTYY | |
| 5 | Variant 2 | ADSVKGRFTVSRDNSKNTLY | |
| | (QE, SA) | LQMNSLRAEDTALYYCAKDL | |
| | | GGYNYGLFDYWGQGTLVTV | |
| | | SS (SEQ ID NO: 12) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADSVKG | |
| | | (SEQ ID NO: 14)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNSGGSTYY | |
| 5 | Variant 3 | ADAVKGRFTVSRDNSKNTLY | |
| | (QE, SA) | LQMNSLRAEDTALYYCAKDL | |
| | | GGYNYGLFDYWGQGTLVTV | |
| | | SS (SEQ ID NO: 15) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADAVKG | |
| | | (SEQ ID NO: 16)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNSGGSTYY | |
| 5 | Variant | ADSVKGRFTVSRDNAKNTLY | |
| | 4(QE, SA) | LQMNSLRAEDTALYYCAKDL | |
| | | GGYNYGLFDYWGQGTLVTV | |
| | | SS (SEQ ID NO: 17) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNSGGSTYY | |
| 5 | Variant 5 | ADSVKGRFTVSRDNSKQTLY | |
| | (QE, NQ) | LQMNSLRAEDTALYYCAKDL | |
| | | GGYNYGLFDYWGQGTLVTV | |
| | | SS (SEQ ID NO: 18) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNSGGSTYY | |
| 5 | Variant 6 | ADSVKGRFTVSRDNSKNTLY | |
| | (QE, NQ) | LQMQSLRAEDTALYYCAKDL | |
| | | GGYNYGLFDYWGQGTLVTV | |
| | | SS (SEQ ID NO: 19) | |
| | | CDR1: GFTFSSY (SEQ ID | |
| | | NO: 2)(Chothia) or SYAMS | |
| | | (SEQ ID NO: 3)(Kabat) | |
| | | CDR2: FNSGGS (SEQ ID NO: 4) | |
| | | (Chothia) or | |
| | | AIFNSGGSTYYADSVKG (SEQ | |
| | | ID NO: 5)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNAGGSTYY | |
| 5 | Variant 7 | ADAVKGRFTVSRDNSKNTLY | |
| | (QE, | LQMNSLRAEDTALYYCAKDL | |
| | SA, SA) | GGYNYGLFDYWGQGTLVTV | |
| | | SS (SEQ ID NO: 20) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| | PH_420- | EVQLVQSGGGLVQPGGSLRL | |
| | CEACAM5_ | SCAASGFTFSSYAMSWVRQA | |
| | VH_ | PGKGLEWVSAIFNAGGSTYY | |
| | Variant 8 | ADAVKGRFTVSRDNAKNTLY | |
| | (QA, SA, | LQMNSLRAEDTALYYCAKDL | |
| | SA, SA) | GGYNYGLFDYWGQGTLVTV | |
| | | SS (SEQ ID NO: 22) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNAGGSTYY | |
| 5 | Variant 9 | ADAVKGRFTVSRDNAKQTLY | |
| | (QE, SA, | LQMNSLRAEDTALYYCAKDL | |
| | SA, SA, | GGYNYGLFDYWGQGTLVTV | |
| | NQ) | SS (SEQ ID NO: 23) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| Clone | PH_420- | EVQLVQSGGGLVQPGGSLRL | |
| PH_420- | CEACAM5_ | SCAASGFTFSSYAMSWVRQA | |
| CEACAM5 | VH_ | PGKGLEWVSAIFNAGGSTYY | |
| 5 | Variant 10 | ADAVKGRFTVSRDNAKQTLY | |
| | (QE, SA, | LQMQSLRAEDTALYYCAKDL | |
| | SA, SA, | GGYNYGLFDYWGQGTLVTV | |
| | NQ, NQ) | SS (SEQ ID NO: 24) | |
| | | CDR1: GFTFSSY (SEQ ID | |
| | | NO: 2)(Chothia) or SYAMS | |
| | | (SEQ ID NO: 3)(Kabat) | |
| | | CDR2: FNAGGS (SEQ ID | |
| | | NO: 13)(Chothia) or | |
| | | AIFNAGGSTYYADAVKG | |
| | | (SEQ ID NO: 21)(Kabat) | |
| | | CDR3: DLGGYNYGLFDY | |
| | | (SEQ ID NO: 6) | |
| Clone | PH_420- | | DIRMTQSPSTLSASVGDR |
| PH_420- | CEACAM5_ | | VTITCWASQSISSWLAWY |
| CEACAM5 | VL_ | | QQKPGKAPKLLIYKASSL |
| | Variant 1 | | ESGVPSRFSGSGSGTEFTL |
| | (DN) | | TISSLQPDNFATYYCQQY |
| | | | NSYSYTFGQGTKLEIK |
| | | | (SEQ ID NO: 25) |
| | | | CDR1: WASQSISSWLA |
| | | | (SEQ ID NO: 7) |
| | | | CDR2: KASSLES (SEQ ID |
| | | | NO: 8) |
| | | | CDR3: QQYNSYSYT (SEQ |
| | | | ID NO: 9) |
| Clone | PH_420- | | DIRMTQSPSTLSASVGDR |
| PH_420- | CEACAM5_ | | VTITCWASQSISSWLAWY |
| CEACAM5 | VL_ | | QQKPGKAPKLLIYKASSL |
| 5 | Variant 2 | | ESGVPSRFSGSGSGTEFTL |
| | (SA) | | TISSLQPDDFATYYCQQY |
| | | | NAYSYTFGQGTKLEIK |
| | | | (SEQ ID NO: 26) |
| | | | CDR1: WASQSISSWLA |
| | | | (SEQ ID NO: 7) |
| | | | CDR2: KASSLES (SEQ ID |
| | | | NO: 8) |
| | | | CDR3: QQYNAYSYT (SEQ |
| | | | ID NO: 27) |
| Clone | PH_420- | | DIRMTQSPSTLSASVGDR |
| PH_420- | CEACAM5_ | | VTITCWASQSISSWLAWY |
| CEACAM5 | VL_ | | QQKPGKAPKLLIYKASSL |
| 5 | Variant 3 | | ESGVPSRFSGSGSGTEFTL |
| | (DN, SA) | | TISSLQPDNFATYYCQQY |
| | | | NAYSYTFGQGTKLEIK |
| | | | (SEQ ID NO: 28) |
| | | | CDR1: WASQSISSWLA |
| | | | (SEQ ID NO: 7) |
| | | | CDR2: KASSLES (SEQ ID |
| | | | NO: 8) |
| | | | CDR3: QQYNAYSYT (SEQ |
| | | | ID NO: 27) |
| Clone | PH_420- | | DIQMTQSPSTLSASVGDR |
| PH_420- | CEACAM5_ | | VTITCWASQSISSWLAWY |
| CEACAM5 | VL_ | | QQKPGKAPKLLIYKASSL |
| | Variant 4 | | ESGVPSRFSGSGSGTEFTL |
| | (R(L3)Q) | | TISSLQPDDFATYYCQQY |
| | | | NSYSYTFGQGTKLEIK |
| | | | (SEQ ID NO: 637) |
| | | | CDR1: WASQSISSWLA |
| | | | (SEQ ID NO: 7) |
| | | | CDR2: KASSLES (SEQ ID |
| | | | NO: 8) |
| | | | CDR3: QQYNSYSYT (SEQ |
| | | | ID NO: 9) |
| Clone | Clone | QVQLVQSGGGLVQPGGSQRL | DIQLTQSPATLSVSPGERA |
| 1078_C04 | 1078_C04 | SCAASGFTFTSYAMSWVRQA | TLSCRASQSVSSSYLAWY |
| CEACAM5_ | CEACAM5_ | PGKGLEWVSAISGTGDSTFYA | QQKPGQAPRLLIYGASSR |
| VH_ | VH_ | DSVKGRFTFSRDNSKNTLYL | ATGIPDRFSGSGSGTDFTL |
| | | QMNSLRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 34) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFYADSVKG (SEQ | ID NO: 42) |
| | | ID NO: 37)(Kabat) | |
| | | CDR3: DLGWLQYGLFDY | |
| | | (SEQ ID NO: 38) | |
| Clone | 1078_C04 | EVQLVQSGGGLVQPGGSQRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGDSTFYA | QQKPGQAPRLLIYGASSR |
| VH | Variant 1 | DSVKGRFTFSRDNSKNTLYL | ATGIPDRFSGSGSGTDFTL |
| | (QE) | QMNSLRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 43) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFY ADSVKG (SEQ | ID NO: 42) |
| | | ID NO: 37)(Kabat) | |
| | | CDR3: DLGWLQYGLFDY | |
| | | (SEQ ID NO: 38) | |
| Clone | 1078_C04 | EVQLVQSGGGLVQPGGSQRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGESTFYA | QQKPGQAPRLLIYGASSR |
| VH | Variant 2 | DSVKGRFTFSRDNSKNTLYL | ATGIPDRFSGSGSGTDFTL |
| | (QE, DE) | QMNSLRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 44) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGES (SEQ ID | NO: 41) |
| | | NO: 45)(Chothia) or | CDR3: QQYNNWPLT (SEQ |
| | | AISGTGESTFY ADSVKG (SEQ | ID NO: 42) |
| | | ID NO: 46)(Kabat) | |
| | | CDR3: DLGWLQYGLFDY | |
| | | (SEQ ID NO: 38) | |
| Clone | 1078_C04 | EVQLVQSGGGLVQPGGSQRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGDSTFYA | QQKPGQAPRLLIYGASSR |
| VH | Variant 3 | DAVKGRFTFSRDNSKNTLYL | ATGIPDRFSGSGSGTDFTL |
| | (QE, SA) | QMNSLRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 47) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFYADAVKG (SEQ | ID NO: 42) |
| | | ID NO: 48)(Kabat) | |
| | | CDR3: DLGWLQYGLFDY | |
| | | (SEQ ID NO: 38) | |
| Clone | 1078_C04 | EVQLVQSGGGLVQPGGSQRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGDSTFYA | QQKPGQAPRLLIYGASSR |
| VH | Variant 4 | DSVKGRFTFSRENSKNTLYLQ | ATGIPDRFSGSGSGTDFTL |
| | (QE, DE) | MNSLRAEDTAVYYCAKDLG | TISRLEPEDFAVYYCQQY |
| | | WLQYGLFDYWGQGTLVTVS | NNWPLTFGGGTKVEIK |
| | | S (SEQ ID NO: 49) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFYADSVKG (SEQ | ID NO: 42) |
| | | ID NO: 37)(Kabat) | |
| | | CDR3: DLGWLQYGLFDY | |
| | | (SEQ ID NO: 38) | |
| Clone | 1078_C04 | QVQLVQSGGGLVQPGGSQRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGDSTFYA | QQKPGQAPRLLIYGASSR |
| VH | Variant 5 | DSVKGRFTFSRDNAKNTLYL | ATGIPDRFSGSGSGTDFTL |
| | (QE, SA) | QMNSLRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 50) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFYADSVKG (SEQ | ID NO: 42) |
| | | ID NO: 37)(Kabat) | |
| | | CDR3: DLGWLQYGLFDY | |
| | | (SEQ ID NO: 38) | |
| Clone | 1078_C04 | EVQLVQSGGGLVQPGGSQRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGDSTFYA | QQKPGQAPRLLIYGASSR |
| VH | Variant 6 | DSVKGRFTFSRDNSKNTLYL | ATGIPDRFSGSGSGTDFTL |
| | (QE, SA) | QMNALRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 51) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFYADSVKG (SEQ | ID NO: 42) |
| | | ID NO: 37)(Kabat) | |
| | | CDR3: DLGWLQYGLFDY | |
| | | (SEQ ID NO: 38) | |
| Clone | 1078_C04 | QVQLVQSGGGLVQPGGSLRL | DIQLTQSPATLSVSPGERA |
| 1078_C04 | CEACAM5_ | SCAASGFTFTSYAMSWVRQA | TLSCRASQSVSSSYLAWY |
| CEACAM5_ | VH_ | PGKGLEWVSAISGTGDSTFYA | QQKPGQAPRLLIYGASSR |
| VH | Variant 7 | DSVKGRFTFSRDNSKNTLYL | ATGIPDRFSGSGSGTDFTL |
| | (Q(H18)L) | QMNSLRAEDTAVYYCAKDL | TISRLEPEDFAVYYCQQY |
| | | GWLQYGLFDYWGQGTLVTV | NNWPLTFGGGTKVEIK |
| | | SS (SEQ ID NO: 638) | (SEQ ID NO: 39) |
| | | CDR1: GFTFTSY (SEQ ID | CDR1: RASQSVSSSYLA |
| | | NO: 35)(Chothia) or SYAMS | (SEQ ID NO: 40) |
| | | (SEQ ID NO: 3)(Kabat) | CDR2: GASSRAT (SEQ ID |
| | | CDR2: SGTGDS (SEQ ID | NO: 41) |
| | | NO: 36)(Chothia) or | CDR3: QQYNNWPLT (SEQ |
| | | AISGTGDSTFYADSVKG (SEQ | ID NO: 42) |
| | | ID NO: 37)(Kabat) | |
| | | CDR3: DLGWLQYGLFDY | |
| | | (SEQ ID NO: 38) | |
| Clone | Clone | QVQLQESGPGLVRPSGTLSLT | DIVMTQTPATLSASVGDR |
| 1078_C04 | 1079_H05 | CAVSGGSISSPTWWSWVRQP | VTITCRASQSVRSNLAWY |
| CEACAM5_ | CEACAM5_ | PGKGLEWIGEIHPSGRTNYNP | QQKPGQAPRLLIYGASTR |
| VH | VH | SLKSRVTISVDKSKNQFSLKL | ATGIPARFSGSGSGTEFTL |
| | | GSVTAADTAVYYCAREGFYY | TISSLQSEDFAVYYCQQY |
| | | GSGNYYYFDYWGQGTLVTV | NNWPTFGQGTRLEIK |
| | | SS (SEQ ID NO: 55) | (SEQ ID NO: 61) |
| | | CDR1: GGSISSPT (SEQ ID | CDR1: RASQSVRSNLA |
| | | NO: 56)(Chothia) or SPTWWS | (SEQ ID NO: 62) |
| | | (SEQ ID NO: 57)(Kabat) | CDR2: GASTRAT (SEQ ID |
| | | CDR2: HPSGR (SEQ ID NO: 58) | NO: 63) |
| | | (Chothia) or | CDR3: QQYNNWPT (SEQ |
| | | EIHPSGRTNYNPSLKS (SEQ ID | ID NO: 64) |
| | | NO: 59)(Kabat) | |
| | | CDR3: EGFYYGSGNYYYFDY | |
| | | (SEQ ID NO: 60) | |
| Clone | 1079_H05 | EVQLQESGPGLVRPSGTLSLT | DIVMTQTPATLSASVGDR |
| 1078_C04 | CEACAM5_ | CAVSGGSISSPTWWSWVRQP | VTITCRASQSVRSNLAWY |
| CEACAM5_ | VH_ | PGKGLEWIGEIHPSGRTNYNP | QQKPGQAPRLLIYGASTR |
| VH | Variant 1 | SLKSRVTISVDKSKNQFSLKL | ATGIPARFSGSGSGTEFTL |
| | (QE) | GSVTAADTAVYYCAREGFYY | TISSLQSEDFAVYYCQQY |
| | | GSGNYYYFDYWGQGTLVTV | NNWPTFGQGTRLEIK |
| | | SS (SEQ ID NO: 65) | (SEQ ID NO: 61) |
| | | CDR1: GGSISSPT (SEQ ID | CDR1: RASQSVRSNLA |
| | | NO: 56)(Chothia) or SPTWWS | (SEQ ID NO: 62) |
| | | (SEQ ID NO: 57)(Kabat) | CDR2: GASTRAT (SEQ ID |
| | | CDR2: HPSGR (SEQ ID NO: 58) | NO: 63) |
| | | (Chothia) or | CDR3: QQYNNWPT (SEQ |
| | | EIHPSGRTNYNPSLKS (SEQ ID | ID NO: 64) |
| | | NO: 59)(Kabat) | |
| | | CDR3: EGFYYGSGNYYYFDY | |
| | | (SEQ ID NO: 60) | |
| 7A10.A7- | 7A10.A7- | QVQLVESGGDVVQPGRSLRL | EIVMTQSPLSLPVTPGEPA |
| CEACAM5- | CEACAM5- | SCAASGFILSNYGMHWVRQA | SISCRSSQSLLHSNGYNYL |
| B.01 | B.01 | PGKGLEWVAAMWYDGSNN | DWYLQKPGQSPQLLISLG |
| | | YYEDSVKGRFTISRDNSKNTL | SIRASGVPDRFSGSGSGTN |
| | | YLQMNSLRAEDTAVYYCARE | FTLTISRVEAEDVGFYYC |
| | | RVSRHFDWHYYYGMDVWG | MQALQTPRTFGQGTKVDI |
| | | QGTTVTVSS (SEQ ID NO: 67) | T (SEQ ID NO: 73) |
| | | CDR1: GFILSNY (SEQ ID | CDR1: |
| | | NO: 68)(Chothia) or NYGMH | RSSQSLLHSNGYNYLD |
| | | (SEQ ID NO: 69)(Kabat) | (SEQ ID NO: 74) |
| | | CDR2: WYDGSN (SEQ ID | CDR2: LGSIRAS (SEQ ID |
| | | NO: 70)(Chothia) or | NO: 75) |
| | | AMWYDGSNNYYEDSVKG | CDR3: MQALQTPRT (SEQ |
| | | (SEQ ID NO: 71)(Kabat) | ID NO: 76) |
| | | CDR3: | |
| | | ERVSRHFDWHYYYGMDV | |
| | | (SEQ ID NO: 72) | |
| 7A10.A7- | 7A10.A7- | EVQLVESGGDVVQPGRSLRL | |
| CEACAM5- | CEACAM5- | SCAASGFILSNYGMHWVRQA | |
| B.01 | B.01_ | PGKGLEWVAAMWYDGSNN | |
| | VH_ | YYEDSVKGRFTISRDNSKNTL | |
| | Variant | YLQMNSLRAEDTAVYYCARE | |
| | 1_(QE) | RVSRHFDWHYYYGMDVWG | |
| | | QGTTVTVSS (SEQ ID NO: 77) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 7A10.A7- | 7A10.A7- | EVQLVESGGDVVQPGRSLRL | |
| CEACAM5- | CEACAM5- | SCAASGFILSNYGMHWVRQA | |
| B.01 | B.01_ | PGKGLEWVAAMWYDGSNN | |
| | VH_ | YYEDTVKGRFTISRDNSKNTL | |
| | Variant | YLQMNSLRAEDTAVYYCARE | |
| | 2_(QE, ST) | RVSRHFDWHYYYGMDVWG | |
| | | QGTTVTVSS (SEQ ID NO: 78) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDTVKG | |
| | | (SEQ ID NO: 79)(Kabat) | |
| | | CDR3: | |
| | | ERVSRHFDWHYYYGMDV | |
| | | (SEQ ID NO: 72) | |
| 7A10.A7- | 7A10.A7- | EVQLVESGGDVVQPGRSLRL | |
| CEACAM5- | CEACAM5- | SCAASGFILSNYGMHWVRQA | |
| B.01 | B.01_ | PGKGLEWVAAMWYDGSNN | |
| | VH_ | YYEDSVKGRFTISRDQSKNTL | |
| | Variant | YLQMNSLRAEDTAVYYCARE | |
| | 3_(QE, | RVSRHFDWHYYYGMDVWG | |
| | NQ) | QGTTVTVSS (SEQ ID NO: 80) | |
| | VH | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 7A10.A7- | 7A10.A7- | EVQLVESGGDVVQPGRSLRL | |
| CEACAM5- | CEACAM5- | SCAASGFILSNYGMHWVRQA | |
| B.01 | B.01_ | PGKGLEWVAAMWYDGSNN | |
| | VH_ | YYEDSVKGRFTISRDNSKQTL | |
| | Variant | YLQMNSLRAEDTAVYYCARE | |
| | 4_(QE, | RVSRHFDWHYYYGMDVWG | |
| | NQ) | QGTTVTVSS (SEQ ID NO: 81) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 7A10.A7- | 7A10.A7- | QVQLVESGGDVVQPGRSLRL | |
| CEACAM5- | CEACAM5- | SCAASGFILSNYGMHWVRQA | |
| B.01 | B.01_ | PGKGLEWVAAMWYDGSNN | |
| | VH_ | YYEDSVKGRFTISRDNSKNTL | |
| | Variant | YLQMQSLRAEDTAVYYCARE | |
| | 5_(QE, | RVSRHFDWHYYYGMDVWG | |
| | NQ) | QGTTVTVSS (SEQ ID NO: 82) | |
| | | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 7A10.A7- | 7A10.A7- | EVQLVESGGDVVQPGRSLRL | |
| CEACAM5- | CEACAM5- | SCAASGFILSNYGMHWVRQA | |
| B.01 | B.01_ | PGKGLEWVAAMWYDGSNN | |
| | VH_ | YYEDTVKGRFTISRDQSKQTL | |
| | Variant | YLQMQSLRAEDTAVYYCARE | |
| | 6_(QE,ST, | RVSRHFDWHYYYGMDVWG | |
| | NQ, NQ, | QGTTVTVSS (SEQ ID NO: 83) | |
| | NQ) | CDR1: GFILSNY (SEQ ID | |
| | | NO: 68)(Chothia) or NYGMH | |
| | | (SEQ ID NO: 69)(Kabat) | |
| | | CDR2: WYDGSN (SEQ ID | |
| | | NO: 70)(Chothia) or | |
| | | AMWYDGSNNYYEDTVKG | |
| | | (SEQ ID NO: 79)(Kabat) | |
| | | CDR3: | |
| | | ERVSRHFDWHYYYGMDV | |
| | | (SEQ ID NO: 72) | |
| 7A10.A7- | 7A10.A7- | | EIVMTQSPLSLPVTPGEPA |
| CEACAM5- | CEACAM5- | | SISCRSSQSLLHSNGYNYL |
| B.01 | B.01- | | DWYLQKPGQSPQLLISLG |
| | N(L70)D | | SIRASGVPDRFSGSGSGTD |
| | VL | | FTLTISRVEAEDVGFYYC |
| | | | MQALQTPRTFGQGTKVDI |
| | | | T (SEQ ID NO: 84) |
| | | | CDR1: |
| | | | RSSQSLLHSNGYNYLD |
| | | | (SEQ ID NO: 74) |
| | | | CDR2: LGSIRAS (SEQ ID |
| | | | NO: 75) |
| | | | CDR3: MQALQTPRT (SEQ |
| | | | ID NO: 76) |
| 7A10.A7- | 7A10.A7- | | EIVMTQSPLSLPVTPGEPA |
| CEACAM5- | CEACAM5- | | SISCRSSQSLLHSNGYNYL |
| B.01 | B.01- | | DWYLQKPGQSPQLLISLG |
| | N(L70)Q | | SIRASGVPDRFSGSGSGTQ |
| | VL | | FTLTISRVEAEDVGFYYC |
| | | | MQALQTPRTFGQGTKVDI |
| | | | T (SEQ ID NO: 85) |
| | | | CDR1: |
| | | | RSSQSLLHSNGYNYLD |
| | | | (SEQ ID NO: 74) |
| | | | CDR2: LGSIRAS (SEQ ID |
| | | | NO: 75) |
| | | | CDR3: MQALQTPRT (SEQ |
| | | | ID NO: 76) |
| 7A10.A7- | 7A10.A7- | | EIVMTQSPLSLPVTPGEPA |
| CEACAM5- | CEACAM5- | | SISCRSSQSLLHSNGYNYL |
| B.01 | B.01_ | | DWYLQKPGQSPQLLISLG |
| | VL_ | | SIRASGVPDRFSGSGSGTN |
| | Variant | | FALTISRVEAEDVGFYYC |
| | 1_(TA) | | MQALQTPRTFGQGTKVDI |
| | | | T (SEQ ID NO: 86) |
| | | | CDR1: |
| | | | RSSQSLLHSNGYNYLD |
| | | | (SEQ ID NO: 74) |
| | | | CDR2: LGSIRAS (SEQ ID |
| | | | NO: 75) |
| | | | CDR3: MQALQTPRT (SEQ |
| | | | ID NO: 76) |
| 7A10.A7- | 7A10.A7- | | EIVMTQSPLSLPVTPGEPA |
| CEACAM5- | CEACAM5- | | SISCRSSQSLLHSSGYNYL |
| B.01 | B.01- | | DWYLQKPGQSPQLLISLG |
| | N(L70)D, N | | SIRASGVPDRFSGSGSGTD |
| | (L28)S VL | | FTLTISRVEAEDVGFYYC |
| | | | MQALQTPRTFGQGTKVDI |
| | | | T (SEQ ID NO: 631) |
| | | | CDR1: |
| | | | RSSQSLLHSSGYNYLD |
| | | | (SEQ ID NO: 632) |
| | | | CDR2: LGSIRAS (SEQ ID |
| | | | NO: 75) |
| | | | CDR3: MQALQTPRT (SEQ |
| | | | ID NO: 76) |
| 7A10.A7- | 7A10.A7- | | EIVMTQSPLSLPVTPGEPA |
| CEACAM5- | CEACAM5- | | SISCRSSQSLLHSQGYNYL |
| B.01 | B.01- | | DWYLQKPGQSPQLLISLG |
| | N(L70)D, N | | SIRASGVPDRFSGSGSGTD |
| | (L28)Q VL | | FTLTISRVEAEDVGFYYC |
| | | | MQALQTPRTFGQGTKVDI |
| | | | T (SEQ ID NO: 633) |
| | | | CDR1: |
| | | | RSSQSLLHSQGYNYLD |
| | | | (SEQ ID NO: 634) |
| | | | CDR2: LGSIRAS (SEQ ID |
| | | | NO: 75) |
| | | | CDR3: MQALQTPRT (SEQ |
| | | | ID NO: 76) |
| 7A10.A7- | 7A10.A7- | | EIVMTQSPLSLPVTPGEPA |
| CEACAM5- | CEACAM5- | | SISCRSSQSLLHSNAYNYL |
| B.01 | B.01- | | DWYLQKPGQSPQLLISLG |
| | N(L70) | | SIRASGVPDRFSGSGSGTD |
| | D, G | | FTLTISRVEAEDVGFYYC |
| | (L29)A VL | | MQALQTPRTFGQGTKVDI |
| | | | T (SEQ ID NO: 635) |
| | | | CDR1: |
| | | | RSSQSLLHSNAYNYLD |
| | | | (SEQ ID NO: 636) |
| | | | CDR2: LGSIRAS (SEQ ID |
| | | | NO: 75) |
| | | | CDR3: MQALQTPRT (SEQ |
| | | | ID NO: 76) |
| 8H2.B10- | 8H2.B10- | QVQLVESGGDVVQPGRSLRL | EIVMTQSPLSLPVTPGEPA |
| CEACAM5- | CEACAM5- | SCAASGFTLSSYGMHWVRQA | SISCRSSQSLLHYNGYNYL |
| B.01 | B.01 | PGKGLEWVAAMWYDGSNN | DWYLQKPGQSPQLLISLG |
| | | YYEDSVKGRFTISRDNSKNTL | SIRASGVPDRFSGSGSGTN |
| | | YLQMNSLRAEDTAVYYCARE | FTLTISRVEAEDVGFYYC |
| | | RVSRHFDWHYYYGMDVWG | MQALQTPRTFGQGTKVDI |
| | | QGTTVTVSS (SEQ ID NO: 90) | T (SEQ ID NO: 93) |
| | | CDR1: GFTLSSY (SEQ ID | CDR1: |
| | | NO: 91)(Chothia) or SYGMH | RSSQSLLHYNGYNYLD |
| | | (SEQ ID NO: 92)(Kabat) | (SEQ ID NO: 94) |
| | | CDR2: WYDGSN (SEQ ID | CDR2: LGSIRAS (SEQ ID |
| | | NO: 70)(Chothia) or | NO: 75) |
| | | AMWYDGSNNYYEDSVKG | CDR3: MQALQTPRT (SEQ |
| | | (SEQ ID NO: 71)(Kabat) | ID NO: 76) |
| | | CDR3: | |
| | | ERVSRHFDWHYYYGMDV | |
| | | (SEQ ID NO: 72) | |
| 8H2.B10- | 8H2.B10- | | EIVMTQSPLSLPVTPGEPA |
| CEACAM5- | CEACAM5- | | SISCRSSQSLLHYNGYNYL |
| B.01 | B.01- | | DWYLQKPGQSPQLLISLG |
| | N(L70)D | | SIRASGVPDRFSGSGSGTD |
| | VL | | FTLTISRVEAEDVGFYYC |
| | | | MQALQTPRTFGQGTKVDI |
| | | | T (SEQ ID NO: 95) |
| | | | CDR1: |
| | | | RSSQSLLHYNGYNYLD |
| | | | (SEQ ID NO: 94) |
| | | | CDR2: LGSIRAS (SEQ ID |
| | | | NO: 75) |
| | | | CDR3: MQALQTPRT (SEQ |
| | | | ID NO: 76) |
| 8H2.B10- | 8H2.B10- | | EIVMTQSPLSLPVTPGEPA |
| CEACAM5- | CEACAM5- | | SISCRSSQSLLHYNGYNYL |
| B.01 | B.01- | | DWYLQKPGQSPQLLISLG |
| | N(L70)Q | | SIRASGVPDRFSGSGSGTQ |
| | VL | | FTLTISRVEAEDVGFYYC |
| | | | MQALQTPRTFGQGTKVDI |
| | | | T (SEQ ID NO: 96) |
| | | | CDR1: |
| | | | RSSQSLLHYNGYNYLD |
| | | | (SEQ ID NO: 94) |
| | | | CDR2: LGSIRAS (SEQ ID |
| | | | NO: 75) |
| | | | CDR3: MQALQTPRT (SEQ |
| | | | ID NO: 76) |
| 8H2.B10- | VH_ | | EIVMTQSPLSLPVTPGEPA |
| CEACAM5- | 8H2.B10- | | SISCRSSQSLLHYNGYNYL |
| B.01 | CEACAM5- | | DWYLQKPGQSPQLLISLG |
| | B.01- | | SIRASGVPDRFSGSGSGTN |
| | T(L72)A | | FALTISRVEAEDVGFYYC |
| | VL | | MQALQTPRTFGQGTKVDI |
| | | | T (SEQ ID NO: 97) |
| | | | CDR1: |
| | | | RSSQSLLHYNGYNYLD |
| | | | (SEQ ID NO: 94) |
| | | | CDR2: LGSIRAS (SEQ ID |
| | | | NO: 75) |
| | | | CDR3: MQALQTPRT (SEQ |
| | | | ID NO: 76) |
| 8H2.B10- | VH_ | EVQLVESGGDVVQPGRSLRL | |
| CEACAM5- | 8H2.B10- | SCAASGFTLSSYGMHWVRQA | |
| B.01 | CEACAM5- | PGKGLEWVAAMWYDGSNN | |
| | B.01 | YYEDSVKGRFTISRDNSKNTL | |
| | (QH1E) | YLQMNSLRAEDTAVYYCARE | |
| | | RVSRHFDWHYYYGMDVWG | |
| | | QGTTVTVSS (SEQ ID NO: 332) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 8H2.B10- | VH_ | QVQLVESGGDVVQPGRSLRL | |
| CEACAM5- | 8H2.B10- | SCAASGFTLSSYGMHWVRQA | |
| B.01 | CEACAM5- | PGKGLEWVAAMWYDGSNN | |
| | B.01 | YYEDTVKGRFTISRDNSKNTL | |
| | (SH62T) | YLQMNSLRAEDTAVYYCARE | |
| | | RVSRHFDWHYYYGMDVWG | |
| | | QGTTVTVSS (SEQ ID NO: 333) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDTVKG | |
| | | (SEQ ID NO: 79)(Kabat) | |
| | | CDR3: | |
| | | ERVSRHFDWHYYYGMDV | |
| | | (SEQ ID NO: 72) | |
| 8H2.B10- | VH_8H2.B10- | QVQLVESGGDVVQPGRSLRL | |
| CEACAM5- | CEACAM5- | SCAASGFTLSSYGMHWVRQA | |
| B.01 | B.01 | PGKGLEWVAAMWYDGSNN | |
| | (NH73Q) | YYEDSVKGRFTISRDQSKNTL | |
| | | YLQMNSLRAEDTAVYYCARE | |
| | | RVSRHFDWHYYYGMDVWG | |
| | | QGTTVTVSS (SEQ ID NO: 334) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 8H2.B10- | VH_8H2.B10- | QVQLVESGGDVVQPGRSLRL | |
| CEACAM5- | CEACAM5- | SCAASGFTLSSYGMHWVRQA | |
| B.01 | B.01 | PGKGLEWVAAMWYDGSNN | |
| | (NH76Q) | YYEDSVKGRFTISRDNSKQTL | |
| | | YLQMNSLRAEDTAVYYCARE | |
| | | RVSRHFDWHYYYGMDVWG | |
| | | QGTTVTVSS (SEQ ID NO: 335) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| 8H2.B10- | VH_8H2.B10- | QVQLVESGGDVVQPGRSLRL | |
| CEACAM5- | CEACAM5- | SCAASGFTLSSYGMHWVRQA | |
| B.01 | B.01 | PGKGLEWVAAMWYDGSNN | |
| | (NH82aQ) | YYEDSVKGRFTISRDNSKNTL | |
| | | YLQMQSLRAEDTAVYYCARE | |
| | | RVSRHFDWHYYYGMDVWG | |
| | | QGTTVTVSS (SEQ ID NO: 336) | |
| | | CDR1: GFTLSSY (SEQ ID | |
| | | NO: 91)(Chothia) or SYGMH | |
| | | (SEQ ID NO: 92)(Kabat) | |
| | | CDR2: WYDGSN (SEQ ID | |
| | | NO: 70)(Chothia) or | |
| | | AMWYDGSNNYYEDSVKG | |
| | | (SEQ ID NO: 71)(Kabat) | |
| | | CDR3: | |
| | | ERVSRHFDWHYYYGMDV | |
| | | (SEQ ID NO: 72) | |
| Murine | Murine | EVQLQQSAAELARPGASVKM | DIVMSQSPSSLAVSVGEK |
| 16F6.A2- | 16F6.A2- | SCKASGYTFTAYTIHWVKQR | VTMSCKSSHSLLYGNFQN |
| CEACAM5- | CEACAM5- | PGQGLEWIGYINPSSGYTEYN | NYLAWYQQKPGQSPKLLI |
| B.02 | B.02 | QKFKDKTTLTADQSSPTAYIQ | YWASTRESGVPDRFTGSG |
| | (VH | LSTLTSEDSAVYYCTREGGLL | SGTDFTLTISSVKAEDLAV |
| | Variant 1) | WFDYWGQGTTLTVST (SEQ | YYCQQYYSYPYTFGGGT |
| | | ID NO: 100) | KLEIK (SEQ ID NO: 106) |
| | | CDR1: GYTFTAY (SEQ ID | CDR1: |
| | | NO: 101)(Chothia) or AYTIH | KSSHSLLYGNFQNNYLA |
| | | (SEQ ID NO: 102)(Kabat) | (SEQ ID NO: 107) |
| | | CDR2: NPSSGY (SEQ ID | CDR2: WASTRES (SEQ ID |
| | | NO: 103)(Chothia) or | NO: 108) |
| | | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Murine | Murine | QVQLQQSAAELARPGASVKM | DIVMSQSPSSLAVSVGEK |
| 16F6.A2- | 16F6.A2- | SCKASGYTFTAYTIHWVKQR | VTMSCKSSHSLLYGNFQN |
| CEACAM5- | CEACAM5- | PGQGLEWIGYINPSSGYTEYN | NYLAWYQQKPGQSPKLLI |
| B.02 | B.02 | QKFKDKTTLTADQSSPTAYIQ | YWASTRESGVPDRFTGSG |
| | (VH | LSTLTSEDSAVYYCTREGGLL | SGTDFTLTISSVKAEDLAV |
| | Variant 2) | WFDYWGQGTTLTVST (SEQ | YYCQQYYSYPYTFGGGT |
| | | ID NO: 110) | KLEIK (SEQ ID NO: 106) |
| | | CDR1: GYTFTAY (SEQ ID | CDR1: |
| | | NO: 101)(Chothia) or AYTIH | KSSHSLLYGNFQNNYLA |
| | | (SEQ ID NO: 102)(Kabat) | (SEQ ID NO: 107) |
| | | CDR2: NPSSGY (SEQ ID | CDR2: WASTRES (SEQ ID |
| | | NO: 103)(Chothia) or | NO: 108) |
| | | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Humanized | Humanized | QVQLVQSGAEVKKPGASVK | DIVMTQSPDSLAVSLGER |
| 16F6.A2- | 16F6.A2- | MSCKASGYTFTAYTIHWVRQ | ATINCKSSHSLLYGNFQN |
| CEACAM5- | CEACAM5- | APGQGLEWIGYINPSSGYTEY | NYLAWYQQKPGQPPKLLI |
| B.02-BM | B.02- | NQKFKDRTTLTADTSIPTAYM | YWASTRESGVPDRFSGSG |
| | BM-H1- | ELSRLRSDDTAVYYCTREGG | SGTDFTLTISSLQAEDVAV |
| | VH | LLWFDYWGQGTLVTVSS | YYCQQYYSYPYTFGGGT |
| | and | (SEQ ID NO: 112) | KLEIK (SEQ ID NO: 119) |
| | Humanized | CDR1: GYTFTAY (SEQ ID | CDR1: |
| | 16F6.A2- | NO: 101)(Chothia) or AYTIH | KSSHSLLYGNFQNNYLA |
| | CEACAM5- | (SEQ ID NO: 102)(Kabat) | (SEQ ID NO: 107) |
| | B.02- | CDR2: NPSSGY (SEQ ID | CDR2: WASTRES (SEQ ID |
| | BM-L1-VL | NO: 103)(Chothia) or | NO: 108) |
| | | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Humanized | Humanized | QVQLVQSGAEVKKPGASVK | DIVMTQSPDSLAVSLGER |
| 16F6.A2- | 16F6.A2- | MSCKASGYTFTAYTIHWVRQ | ATINCKSSHSLLYGNFQN |
| CEACAM5- | CEACAM5- | APGQGLEWIGYINPSSGYTEY | NYLAWYQQKPGQPPKLLI |
| B.02-BM | B.02- | NQKFKDRTTLTADTSIPTAYI | YWASTRESGVPDRFSGSG |
| | BM-H2- | ELSRLRSDDTAVYYCTREGG | SGTDFTLTISSLQAEDVAV |
| | VH | LLWFDYWGQGTLVTVSS | YYCQQYYSYPYTFGGGT |
| | and | (SEQ ID NO: 113) | KLEIK (SEQ ID NO: 119) |
| | Humanized | CDR1: GYTFTAY (SEQ ID | CDR1: |
| | 16F6.A2- | NO: 101)(Chothia) or AYTIH | KSSHSLLYGNFQNNYLA |
| | CEACAM5- | (SEQ ID NO: 102)(Kabat) | (SEQ ID NO: 107) |
| | B.02- | CDR2: NPSSGY (SEQ ID | CDR2: WASTRES (SEQ ID |
| | BM-L1-VL | NO: 103)(Chothia) or | NO: 108) |
| | | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Humanized | 16F6.A2- | EVQLVQSGAEVKKPGASVKV | DIVMTQSPDSLAVSLGER |
| 16F6.A2- | CEACAM5- | SCKASGYTFTAYTIHWVRQA | ATINCKSSHSLLYGNFQN |
| CEACAM5- | B.02- | PGQGLEWMGYINPSSGYTEY | NYLAWYQQKPGQPPKLLI |
| B.02-BM | BM-H1- | NQKFKDRTTLTADTSIPTAYM | YWASTRESGVPDRFSGSG |
| | VH | ELSRLRSDDTAVYYCTREGG | SGTDFTLTISSLQAEDVAV |
| | (Humanized)_ | LLWFDYWGQGTLVTVSS | YYCQQYYSYPYTFGGGT |
| | Variant | (SEQ ID NO: 114) | KLEIK (SEQ ID NO: 119) |
| | 1(QE) and | CDR1: GYTFTAY (SEQ ID | CDR1: |
| | Humanized | NO: 101)(Chothia) or AYTIH | KSSHSLLYGNFQNNYLA |
| | 16F6.A2- | (SEQ ID NO: 102)(Kabat) | (SEQ ID NO: 107) |
| | CEACAM5- | CDR2: NPSSGY (SEQ ID | CDR2: WASTRES (SEQ ID |
| | B.02- | NO: 103)(Chothia) or | NO: 108) |
| | BM-L1-VL | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Humanized | 16F6.A2- | EVQLVQSGAEVKKPGASVKV | DIVMTQSPDSLAVSLGER |
| 16F6.A2- | CEACAM5- | SCKASGYTFTAYTIHWVRQA | ATINCKSSHSLLYGNFQN |
| CEACAM5- | B.02- | PGQGLEWMGYINPSSGYTEY | NYLAWYQQKPGQPPKLLI |
| B.02-BM | BM-H1- | NQKFKDRTTLTADTSIPTAYM | YWASTRESGVPDRFSGSG |
| | VH | ELSRLRSQDTAVYYCTREGG | SGTDFTLTISSLQAEDVAV |
| | (Humanized)_ | LLWFDYWGQGTLVTVSS | YYCQQYYSYPYTFGGGT |
| | Variant | (SEQ ID NO: 115) | KLEIK (SEQ ID NO: 119) |
| | 2(QE) and | CDR1: GYTFTAY (SEQ ID | CDR1: |
| | Humanized | NO: 101)(Chothia) or AYTIH | KSSHSLLYGNFQNNYLA |
| | 16F6.A2- | (SEQ ID NO: 102)(Kabat) | (SEQ ID NO: 107) |
| | CEACAM5- | CDR2: NPSSGY (SEQ ID | CDR2: WASTRES (SEQ ID |
| | B.02- | NO: 103)(Chothia) or | NO: 108) |
| | BM-L1-VL | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Humanized | 16F6.A2- | EVQLVQSGAEVKKPGASVK | DIVMTQSPDSLAVSLGER |
| 16F6.A2- | CEACAM5- | MSCKASGYTFTAYTIHWVRQ | ATINCKSSHSLLYGNFQN |
| CEACAM5- | B.02- | APGQGLEWIGYINPSSGYTEY | NYLAWYQQKPGQPPKLLI |
| B.02-BM | BM-H2- | NQKFKDRTTLTADTSIPTAYI | YWASTRESGVPDRFSGSG |
| | VH | ELSRLRSDDTAVYYCTREGG | SGTDFTLTISSLQAEDVAV |
| | (Humanized)_ | LLWFDYWGQGTLVTVSS | YYCQQYYSYPYTFGGGT |
| | Variant | (SEQ ID NO: 116) | KLEIK (SEQ ID NO: 119) |
| | 1(QE) and | CDR1: GYTFTAY (SEQ ID | CDR1: |
| | Humanized | NO: 101)(Chothia) or AYTIH | KSSHSLLYGNFQNNYLA |
| | 16F6.A2- | (SEQ ID NO: 102)(Kabat) | (SEQ ID NO: 107) |
| | CEACAM5- | CDR2: NPSSGY (SEQ ID | CDR2: WASTRES (SEQ ID |
| | B.02- | NO: 103)(Chothia) or | NO: 108) |
| | BM-L1-VL | YINPSSGYTEYNQKFKD (SEQ | CDR3: QQYYSYPYT (SEQ |
| | | ID NO: 104)(Kabat) | ID NO: 109) |
| | | CDR3: EGGLLWFDY (SEQ ID | |
| | | NO: 105) | |
| Humanized | 16F6.A2- | EVQLVQSGAEVKKPGASVK | DIVMTQSPDSLAVSLGER |
| 16F6.A2- | CEACAM5- | MSCKASGYTFTAYTIHWVRQ | ATINCKSSHSLLYGNFQN |
| CEACAM5- | B.02- | APGQGLEWIGYINPSSGYTEY | NYLAWYQQKPGQPPKLLI |
| B.02-BM | BM-H2- | NQKFKDRTTLTADTSIPTAYI | YWASTRESGVPDRFSGSG |
| | VH | ELSRLRSQDTAVYYCTREGG | SGTDFTLTISSLQAEDVAV |
| | (Humanized)_ | LLWFDYWGQGTLVTVSS | YYCQQYYSYPYTFGGGT |
| | Variant | (SEQ ID NO: 117) | KLEIK (SEQ ID NO: 119) |
| | 2(QH1E, | CDR1: GYTFTAY (SEQ ID | CDR1: |
| | DH85Q) | NO: 101)(Chothia) or AYTIH | KSSHSLLYGNFQNNYLA |
| | and | (SEQ ID NO: 102)(Kabat) | (SEQ ID NO: 107) |
| | Humanized | CDR2: NPSSGY (SEQ ID | CDR2: WASTRES (SEQ ID |
| | 16F6.A2- | NO: 103)(Chothia) or | NO: 108) |
| | CEACAM5- | YINPSSGYTEYNQKFKD (SEQ | CDR3: QQYYSYPYT (SEQ |
| | B.02- | ID NO: 104)(Kabat) | ID NO: 109) |
| | BM-L1-VL | CDR3: EGGLLWFDY (SEQ ID | |
| | | NO: 105) | |

Tier 2

| | | | |
|---|---|---|---|
| PH_415- | PH_415- | QVQLQESGPGLVKPSGTLSLT | EIVMTQSPSSLSASVGDRV |
| CEACAM5 | CEACAM5 | CAVSGGSISSSDWWTWVRQP | TITCRASQGISNYLAWYQ |
| | | PGKGLEWIGEIYHSGSTNYNP | QKPGKVPKLLIYAASTLQ |
| | | SLKSRVTISVDKSKNQFSLNL | SGVPSRFSGSGSGTEFTLTI |
| | | NSVTAADTAVYYCARGYSGS | SSLQPEDFATYYCQQLNS |
| | | YFDLDIWGQGTTVTVSS (SEQ | YPLTFGGGTKVEIK (SEQ |
| | | ID NO: 120) | ID NO: 126) |
| | | CDR1: GGSISSSD (SEQ ID | CDR1: RASQGISNYLA |
| | | NO: 121)(Chothia) or SSDWWT | (SEQ ID NO: 127) |
| | | (SEQ ID NO: 122)(Kabat) | CDR2: AASTLQS (SEQ ID |
| | | CDR2: YHSGS (SEQ ID | NO: 128) |
| | | NO: 123)(Chothia) or | CDR3: QQLNSYPLT (SEQ |
| | | EIYHSGSTNYNPSLKS (SEQ | ID NO: 129) |
| | | ID NO: 124)(Kabat) | |
| | | CDR3: GYSGSYFDLDI (SEQ | |
| | | ID NO: 125) | |
| PH_416- | PH_416- | QVQLQESGPGLVKPSGTLSLT | EIVMTQSPSSLSASVGDRV |
| CEACAM5 | CEACAM5 | CAVSGGSISSSDWWTWVRQP | TITCRASQGISNYLAWYQ |
| | | PGKGLEWIGEIYHSGSTNYNP | QKPGKVPKLLIYAASTLQ |
| | | SLKSRVTISVDKSKNQFSLNL | SGVPSRFSGSGSGTEFTLTI |
| | | NSVTAADTAVYYCARGYSGS | SSLQPEDFATYYCQQLNS |
| | | YFDLDIWGQGTTVTVSS (SEQ | YPLTFGGGTKVEIK (SEQ |
| | | ID NO: 120) | ID NO: 126) |
| | | CDR1: GGSISSSD (SEQ ID | CDR1: RASQGISNYLA |
| | | NO: 121)(Chothia) or SSDWWT | (SEQ ID NO: 127) |
| | | (SEQ ID NO: 122)(Kabat) | CDR2: AASTLQS (SEQ ID |
| | | CDR2: YHSGS (SEQ ID | NO: 128) |
| | | NO: 123)(Chothia) or | CDR3: QQLNSYPLT (SEQ |
| | | EIYHSGSTNYNPSLKS (SEQ | ID NO: 129) |
| | | ID NO: 124)(Kabat) | |
| | | CDR3: GYSGSYFDLDI (SEQ | |
| | | ID NO: 125) | |
| PH_418- | PH_418- | QVQLQESGPGLVKPSGTLSLT | DIRLTQSPSFLSASVGDRV |
| CEACAM5 | CEACAM5 | CAVSGGSISSSDWWTWVRQP | TITCRASQGISSYLAWYQ |
| | | PGKGLEWIGEIYHSGSTNYNP | QKPGKAPKLLIYAASTLQ |
| | | SLKSRVTISVDKSKNQFSLNL | SGVPSRFSGSGSGTEFTLTI |
| | | NSVTAADTAVYYCARGYSGS | SSLQPEDFATYYCQQLNS |
| | | YFDLDIWGQGTTVTVSS (SEQ | YPFTFGPGTKVDIK (SEQ |
| | | ID NO: 120) | ID NO: 130) |
| | | CDR1: GGSISSSD (SEQ ID | CDR1: RASQGISSYLA |
| | | NO: 121)(Chothia) or SSDWWT | (SEQ ID NO: 131) |
| | | (SEQ ID | CDR2: AASTLQS (SEQ ID |
| | | NO: 122)(Kabat) | NO: 128) |
| | | CDR2: YHSGS (SEQ ID | CDR3: QQLNSYPFT (SEQ |
| | | NO: 123)(Chothia) or | ID NO: 132) |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| PH_418- | PH_418- | | DIRLTQSPSFLSASVGDRV |
| CEACAM5 | CEACAM5_ | | TITCRASQGISSYLAWYQ |
| | VL | | QKPGKAPKLLIYAASTLQ |
| | Variant | | SGVPSRFSGSGSGTEFTLTI |
| | | | SSLQPEDFATYYCQQLNA |
| | | | YPFTFGPGTKVDIK (SEQ |
| | | | ID NO: 133) |
| | | | CDR1: RASQGISSYLA |
| | | | (SEQ ID NO: 131) |
| | | | CDR2: AASTLQS (SEQ ID |
| | | | NO: 128) |
| | | | CDR3: QQLNAYPFT (SEQ |
| | | | ID NO: 134) |
| PH_419- | PH_419- | QVQLVQSGPGLVKPSGTLSLT | DIVMTQSPSTLSASVGDR |
| CEACAM5 | CEACAM5 | CAVSGGSISSSDWWTWVRQP | VTITCRASQGISSYLAWY |
| | | PGKGLEWIGEIYHSGSTNYNP | QQKPGKAPKLLIYAASTL |
| | | SLKSRVTISVDKSKNQFSLKL | QSGVPSRFSGSGSGTEFTL |
| | | NSVTAADTAVYYCARGSSGS | TISSLQPEDFATYYCQQLN |
| | | YFDLDIWGQGTTVTVSS (SEQ | SYPLTFGGGTKVEIK (SEQ |
| | | ID NO: 135) | ID NO: 136) |
| | | CDR1: GGSISSSD (SEQ ID | CDR1: RASQGISSYLA |
| | | NO: 121)(Chothia) or SSDWWT | (SEQ ID NO: 131) |
| | | (SEQ ID NO: 122)(Kabat) | CDR2: AASTLQS (SEQ ID |
| | | CDR2: YHSGS (SEQ ID | NO: 128) |
| | | NO: 123)(Chothia) or | CDR3: QQLNSYPLT (SEQ |
| | | EIYHSGSTNYNPSLKS (SEQ | ID NO: 129) |
| | | ID NO: 124)(Kabat) | |
| | | CDR3: GSSGSYFDLDI (SEQ ID | |
| | | NO: 337) | |
| PH_415- | VH | EVQLQESGPGLVKPSGTLSLT | |
| CEACAM5 | PH_415- | CAVSGGSISSSDWWTWVRQP | |
| | CEACAM5 | PGKGLEWIGEIYHSGSTNYNP | |
| | (QH1E) | SLKSRVTISVDKSKNQFSLNL | |
| | | NSVTAADTAVYYCARGYSGS | |
| | | YFDLDIWGQGTTVTVSS (SEQ | |
| | | ID NO: 338) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| PH_415- | VH | QVQLQESGPGLVKPSGTLSLT | |
| CEACAM5 | PH_415- | CAVSGGSISSSDWWTWVRQP | |
| | CEACAM5 | PGKGLEWIGEIYHSGSTNYNP | |
| | (SH82bA) | SLKSRVTISVDKSKNQFSLNL | |
| | | NAVTAADTAVYYCARGYSG | |
| | | SYFDLDIWGQGTTVTVSS | |
| | | (SEQ ID NO: 339) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| PH_415- | VL PH_415_ | | EIVMTQSPSSLSASVGDRV |
| CEACAM5 | CEACAM5 | | TITCRASQGISNYLAWYQ |
| | (SL93A) | | QKPGKVPKLLIYAASTLQ |
| | | | SGVPSRFSGSGSGTEFTLTI |
| | | | SSLQPEDFATYYCQQLNA |
| | | | YPLTFGGGTKVEIK (SEQ |
| | | | ID NO: 340) |
| | | | CDR1: RASQGISNYLA |
| | | | (SEQ ID NO: 127) |
| | | | CDR2: AASTLQS (SEQ ID |
| | | | NO: 128) |
| | | | CDR3: QQLNAYPLT (SEQ |
| | | | ID NO: 341) |
| PH_416- | VH | QVQLQESGPGLVKPSGTLSLT | |
| CEACAM5 | PH_416- | CAVSGGSISSSDWWTWVRQP | |
| | CEACAM5 | PGKGLEWIGEIYHSGSTNYNP | |
| | (SH82bA) | SLKSRVTISVDKSKNQFSLNL | |
| | | NAVTAADTAVYYCARGYSG | |
| | | SYFDLDIWGQGTTVTVSS | |
| | | (SEQ ID NO: 339) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| PH_416- | VL | | EIVMTQSPSSLSASVGDRV |
| CEACAM5 | PH_416- | | TITCRASQGISNYLAWYQ |
| | CEACAM5 | | QKPGKVPKLLIYAASTLQ |
| | (SL93A) | | SGVPSRFSGSGSGTEFTLTI |
| | | | SSLQPEDFATYYCQQLNA |
| | | | YPLTFGGGTKVEIK (SEQ |
| | | | ID NO: 340) |
| | | | CDR1: RASQGISNYLA |
| | | | (SEQ ID NO: 127) |
| | | | CDR2: AASTLQS (SEQ ID |
| | | | NO: 128) |
| | | | CDR3: QQLNAYPLT (SEQ |
| | | | ID NO: 341) |
| PH_418- | VH_PH_418- | QVQLQESGPGLVKPSGTLSLT | |
| CEACAM5 | CEACAM5 | CAVSGGSISSSDWWTWVRQP | |
| | (SH82bA) | PGKGLEWIGEIYHSGSTNYNP | |
| | | SLKSRVTISVDKSKNQFSLNL | |
| | | NAVTAADTAVYYCARGYSG | |
| | | SYFDLDIWGQGTTVTVSS | |
| | | (SEQ ID NO: 339) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| PH_418- | VL | | DIRLTQSPSFLSASVGDRV |
| CEACAM5 | PH_418- | | TITCRASQGISSYLAWYQ |
| | CEACAM5 | | QKPGKAPKLLIYAASTLQ |
| | (SL93A) | | SGVPSRFSGSGSGTEFTLTI |
| | | | SSLQPEDFATYYCQQLNA |
| | | | YPFTFGPGTKVDIK (SEQ |
| | | | ID NO: 133) |
| | | | CDR1: RASQGISSYLA |
| | | | (SEQ ID NO: 131) |
| | | | CDR2: AASTLQS (SEQ ID |
| | | | NO: 128) |
| | | | CDR3: QQLNAYPFT (SEQ |
| | | | ID NO: 134) |
| PH_419- | VH_PH_419- | EVQLVQSGPGLVKPSGTLSLT | |
| CEACAM5 | CEACAM5 | CAVSGGSISSSDWWTWVRQP | |
| | (QH1E) | PGKGLEWIGEIYHSGSTNYNP | |
| | | SLKSRVTISVDKSKNQFSLKL | |
| | | NSVTAADTAVYYCARGSSGS | |
| | | YFDLDIWGQGTTVTVSS (SEQ | |
| | | ID NO: 342) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GSSGSYFDLDI (SEQ ID | |
| | | NO: 337) | |
| PH_419- | VH_PH_419- | QVQLVQSGPGLVKPSGTLSLT | |
| CEACAM5 | CEACAM5 | CAVSGGSISSSDWWTWVRQP | |
| | (SH82bA) | PGKGLEWIGEIYHSGSTNYNP | |
| | | SLKSRVTISVDKSKNQFSLKL | |
| | | NAVTAADTAVYYCARGSSGS | |
| | | YFDLDIWGQGTTVTVSS (SEQ | |
| | | ID NO: 343) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GSSGSYFDLDI (SEQ ID | |
| | | NO: 337) | |
| PH_419- | VL | | DIVMTQSPSTLSASVGDR |
| CEACAM5 | PH_419- | | VTITCRASQGISSYLAWY |
| | CEACAM5 | | QQKPGKAPKLLIYAASTL |
| | (SL93A) | | QSGVPSRFSGSGSGTEFTL |
| | | | TISSLQPEDFATYYCQQLN |
| | | | AYPLTFGGGTKVEIK (SEQ |
| | | | ID NO: 344) |
| | | | CDR1: RASQGISSYLA |
| | | | (SEQ ID NO: 131) |
| | | | CDR2: AASTLQS (SEQ ID |
| | | | NO: 128) |
| | | | CDR3: QQLNAYPLT (SEQ |
| | | | ID NO: 341) |
| PH_417- | PH_417- | QVQLQESGPGLVKPSGTLSLT | DIQVTQSPATLSVSPGERV |
| CEACAM5 | CEACAM5 | CAVSGGSISSSKWWSWVRQS | TLSCRASRSVRSNLAWYQ |
| | | PGKGLEWIGEIFHSGSINHNTS | QKPGQAPRLLIYGASSRA |
| | | FKSRVTISVDKSKNQFSLKLS | TGIPDRFSGSGSGTDFTLTI |
| | | SVTAADTAVYYCARGGSGSY | SSLQPDDFAVYYCQQYNN |
| | | DAFDIWGQGTMVTVSS (SEQ | WPITFGQGTRLEIK (SEQ |
| | | ID NO: 144) | ID NO: 150) |
| | | CDR1: GGSISSSK (SEQ ID | CDR1: RASRSVRSNLA |
| | | NO: 145)(Chothia) or SSKWWS | (SEQ ID NO: 151) |
| | | (SEQ ID NO: 146)(Kabat) | CDR2: GASSRAT (SEQ ID |
| | | CDR2: FHSGS (SEQ ID | NO: 41) |
| | | NO: 147)(Chothia) or | CDR3: QQYNNWPIT (SEQ |
| | | EIFHSGSINHNTSFKS (SEQ ID | ID NO: 152) |
| | | NO: 148)(Kabat) | |
| | | CDR3: GGSGSYDAFDI (SEQ | |
| | | ID NO: 149) | |
| PH_417- | VH_PH_417- | EVQLQESGPGLVKPSGTLSLT | |
| CEACAM5 | CEACAM5 | CAVSGGSISSSKWWSWVRQS | |
| | (QH1E) | PGKGLEWIGEIFHSGSINHNTS | |
| | | FKSRVTISVDKSKNQFSLKLS | |
| | | SVTAADTAVYYCARGGSGSY | |
| | | DAFDIWGQGTMVTVSS (SEQ | |
| | | ID NO: 345) | |
| | | CDR1: GGSISSSK (SEQ ID | |
| | | NO: 145)(Chothia) or SSKWWS | |
| | | (SEQ ID NO: 146)(Kabat) | |
| | | CDR2: FHSGS (SEQ ID | |
| | | NO: 147)(Chothia) or | |
| | | EIFHSGSINHNTSFKS (SEQ ID | |
| | | NO: 148)(Kabat) | |
| | | CDR3: GGSGSYDAFDI (SEQ | |
| | | ID NO: 149) | |
| PH_417- | VH_PH_417- | QVQLQESGPGLVKPSGTLSLT | |
| CEACAM5 | CEACAM5 | CAVSGGSISSSKWWSWVRQS | |
| | (NH60Q) | PGKGLEWIGEIFHSGSINHQTS | |
| | | FKSRVTISVDKSKNQFSLKLS | |
| | | SVTAADTAVYYCARGGSGSY | |
| | | DAFDIWGQGTMVTVSS (SEQ | |
| | | ID NO: 346) | |
| | | CDR1: GGSISSSK (SEQ ID | |
| | | NO: 145)(Chothia) or SSKWWS | |
| | | (SEQ ID NO: 146)(Kabat) | |
| | | CDR2: FHSGS (SEQ ID | |
| | | NO: 147)(Chothia) or | |
| | | EIFHSGSINHQTSFKS (SEQ ID | |
| | | NO: 347)(Kabat) | |
| | | CDR3: GGSGSYDAFDI (SEQ | |
| | | ID NO: 149) | |
| PH_417- | VH_PH_417- | QVQLQESGPGLVKPSGTLSLT | |
| CEACAM5 | CEACAM5 | CAVSGGSISSSKWWSWVRQS | |
| | (SH62A) | PGKGLEWIGEIFHSGSINHNT | |
| | | AFKSRVTISVDKSKNQFSLKL | |
| | | SSVTAADTAVYYCARGGSGS | |
| | | YDAFDIWGQGTMVTVSS | |
| | | (SEQ ID NO: 348) | |
| | | CDR1: GGSISSSK (SEQ ID | |
| | | NO: 145)(Chothia) or SSKWWS | |
| | | (SEQ ID NO: 146)(Kabat) | |
| | | CDR2: FHSGS (SEQ ID | |
| | | NO: 147)(Chothia) or | |
| | | EIFHSGSINHNTAFKS (SEQ ID | |
| | | NO: 349)(Kabat) | |
| | | CDR3: GGSGSYDAFDI (SEQ | |
| | | ID NO: 149) | |
| PH_421- | PH_421- | QVQLQESGPGLVKPSGTLSLT | EIVLTQSPSFLSASVGDRV |
| CEACAM5 | CEACAM5 | CAVSGGSISSSDWWTWVRQP | TITCRASQGISSYLAWYQ |
| | | PGKGLEWIGEIYHSGSTNYNP | QKPGKAPKLLIYAASTLQ |
| | | SLKSRVTISVDKSKNQFSLNL | SGVPSRFSGSGSGTEFTLTI |
| | | NSVTAADTAVYYCARGYSGS | SSLQPEDFATYYCQQLNS |
| | | YFDLDIWGQGTTVTVSSGS | YPFTFGPGTKLEIK (SEQ |
| | | (SEQ ID NO: 155) | ID NO: 156) |
| | | CDR1: GGSISSSD (SEQ ID | CDR1: RASQGISSYLA |
| | | NO: 121)(Chothia) or SSDWWT | (SEQ ID NO: 131) |
| | | (SEQ ID NO: 122)(Kabat) | CDR2: AASTLQS (SEQ ID |
| | | CDR2: YHSGS (SEQ ID | NO: 128) |
| | | NO: 123)(Chothia) or | CDR3: QQLNSYPFT (SEQ |
| | | EIYHSGSTNYNPSLKS (SEQ | ID NO: 132) |
| | | ID NO: 124)(Kabat) | |
| | | CDR3: GYSGSYFDLDI (SEQ | |
| | | ID NO: 125) | |
| PH_421- | PH_421- | | EIVLTQSPSFLSASVGDRV |
| CEACAM5 | CEACAM5_ | | TITCRASQGISSYLAWYQ |
| | VL | | QKPGKAPKLLIYAASTLQ |
| | Variant | | SGVPSRFSGSGSGTEFTLTI |
| | | | SSLQPEDFATYYCQQLNA |
| | | | YPFTFGPGTKLEIK (SEQ |
| | | | ID NO: 157) |
| | | | CDR1: RASQGISSYLA |
| | | | (SEQ ID NO: 131) |
| | | | CDR2: AASTLQS (SEQ ID |
| | | | NO: 128) |
| | | | CDR3: QQLNAYPFT (SEQ |
| | | | ID NO: 134) |
| PH_421- | VH PH_421- | EVQLQESGPGLVKPSGTLSLT | |
| CEACAM5 | CEACAM5 | CAVSGGSISSSDWWTWVRQP | |
| | (QH1E) | PGKGLEWIGEIYHSGSTNYNP | |
| | | SLKSRVTISVDKSKNQFSLNL | |
| | | NSVTAADTAVYYCARGYSGS | |
| | | YFDLDIWGQGTTVTVSSGS | |
| | | (SEQ ID NO: 350) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| PH_421- | VH PH_421- | QVQLQESGPGLVKPSGTLSLT | |
| CEACAM5 | CEACAM5 | CAVSGGSISSSDWWTWVRQP | |
| | (SH82bA) | PGKGLEWIGEIYHSGSTNYNP | |
| | | SLKSRVTISVDKSKNQFSLNL | |
| | | NAVTAADTAVYYCARGYSG | |
| | | SYFDLDIWGQGTTVTVSSGS | |
| | | (SEQ ID NO: 351) | |
| | | CDR1: GGSISSSD (SEQ ID | |
| | | NO: 121)(Chothia) or SSDWWT | |
| | | (SEQ ID NO: 122)(Kabat) | |
| | | CDR2: YHSGS (SEQ ID | |
| | | NO: 123)(Chothia) or | |
| | | EIYHSGSTNYNPSLKS (SEQ | |
| | | ID NO: 124)(Kabat) | |
| | | CDR3: GYSGSYFDLDI (SEQ | |
| | | ID NO: 125) | |
| 1078_G03- | 1078_G03- | QVQLVESGGGLVQPGGSLRL | DIRMTQSPSTLSASVGDR |
| CEACAM5 | CEACAM5 | SCAASGFTFSSYAMSWVRQA | VTITCRASQSISSWLAWY |
| | | PGKGLEWVSEISGSGDRTSYA | QQKPGKAPKLLIYKASSL |
| | | DSVKGRFTISRDNSKNRLYLQ | ESGVPSRFSGSGSGTEFTL |
| | | MNRLRTEDTAVYYCAKDLGP | TISSLQPDDFATYYCQQY |
| | | SGWYGLFDYWGQGTLVTVS | NSYSTWTFGQGTKVDIK |
| | | S (SEQ ID NO: 160) | (SEQ ID NO: 164) |
| | | CDR1: GFTFSSY (SEQ ID | CDR1: RASQSISSWLA |
| | | NO: 2)(Chothia) or SYAMS | (SEQ ID NO: 165) |
| | | (SEQ ID NO: 3)(Kabat) | CDR2: KASSLES (SEQ ID |
| | | CDR2: SGSGDR (SEQ ID | NO: 8) |
| | | NO: 161)(Chothia) or | CDR3: QQYNSYSTWT |
| | | EISGSGDRTSYADSVKG (SEQ | (SEQ ID NO: 166) |
| | | ID NO: 162)(Kabat) | |
| | | CDR3: DLGPSGWYGLFDY | |
| | | (SEQ ID NO: 163) | |
| 1078_G03- | 1078_G03- | | DIRMTQSPSTLSASVGDR |
| CEACAM5 | CEACAM5 | | VTITCRASQSISSWLAWY |
| | VL | | QQKPGKAPKLLIYKASSL |
| | Variant | | ESGVPSRFSGSGSGTEFTL |
| | | | TISSLQPDDFATYYCQQY |
| | | | NA YSTWTFGQGTKVDIK |
| | | | (SEQ ID NO: 167) |
| | | | CDR1: RASQSISSWLA |
| | | | (SEQ ID NO: 165) |
| | | | CDR2: KASSLES (SEQ ID |
| | | | NO: 8) |
| | | | CDR3: QQYNAYSTWT |
| | | | (SEQ ID NO: 168) |
| 1078_G03- | VH)1078_ | EVQLVESGGGLVQPGGSLRL | |
| CEACAM5 | G03- | SCAASGFTFSSYAMSWVRQA | |
| | CEACAM5 | PGKGLEWVSEISGSGDRTSYA | |
| | (QH1E) | DSVKGRFTISRDNSKNRLYLQ | |
| | | MNRLRTEDTAVYYCAKDLGP | |
| | | SGWYGLFDYWGQGTLVTVS | |
| | | S (SEQ ID NO: 352) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| 1078_G03- | VH | QVQLVESGGGLVQPGGSLRL | |
| CEACAM5 | 1078_G03- | SCAASGFTFSSYAMSWVRQA | |
| | CEACAM5 | PGKGLEWVSEISGSGDRTSYA | |
| | (SH62A) | DAVKGRFTISRDNSKNRLYL | |
| | | QMNRLRTEDTAVYYCAKDL | |
| | | GPSGWYGLFDYWGQGTLVT | |
| | | VSS (SEQ ID NO: 353) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADAVKG (SEQ | |
| | | ID NO: 720)(Kabat) | |
| | | CDR3: DLGPSGWYGLFDY | |
| | | (SEQ ID NO: 163) | |
| 1078_G03- | VH | QVQLVESGGGLVQPGGSLRL | |
| CEACAM5 | 1078_G03 | SCAASGFTFSSYAMSWVRQA | |
| | CEACAM5 | PGKGLEWVSEISGSGDRTSYA | |
| | (NH73A) | DSVKGRFTISRDASKNRLYLQ | |
| | | MNRLRTEDTAVYYCAKDLGP | |
| | | SGWYGLFDYWGQGTLVTVS | |
| | | S (SEQ ID NO: 354) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADAVKG (SEQ | |
| | | ID NO: 720)(Kabat) | |
| | | CDR3: DLGPSGWYGLFDY | |
| | | (SEQ ID NO: 163) | |
| 1078_G03- | VH | QVQLVESGGGLVQPGGSLRL | |
| CEACAM5 | 1078_G03 | SCAASGFTFSSYAMSWVRQA | |
| | CEACAM5 | PGKGLEWVSEISGSGDRTSYA | |
| | (SH74A) | DSVKGRFTISRDNAKNRLYL | |
| | | QMNRLRTEDTAVYYCAKDL | |
| | | GPSGWYGLFDYWGQGTLVT | |
| | | VSS (SEQ ID NO: 355) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADAVKG (SEQ | |
| | | ID NO: 720)(Kabat) | |
| | | CDR3: DLGPSGWYGLFDY | |
| | | (SEQ ID NO: 163) | |
| Murine | Murine | EVQLQESGAELVRSGASVKL | QIVLSQSPAIMSASPGEKV |
| 1A1.A3- | 1A1.A3- | SCTASDFNIKDSYMHWVTQR | TITCSTSSSVSYMHWFQQ |
| CEACAM5- | CEACAM5- | PEQGLEWIGWIDPENGDTEY | KPGTSPKLWIYSTSNLASG |
| B.02 | B.02 | APKFQGKATMTADTSSNTAY | VPARFSGSGSGTSYSLTIS |
| | | LHLSSLTSEDTAVYYCNVITT | RMEAEDAATYYCQQRSN |
| | | VVNYAMDYWGQGTSVTVSS | YPLTFGAGTKLELK (SEQ |
| | | (SEQ ID NO: 173) | ID NO: 179) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPENGD (SEQ ID | NO: 181) |
| | | NO: 176)(Chothia) or | CDR3: QQRSNYPLT (SEQ |
| | | WIDPENGDTEYAPKFQG | ID NO: 182) |
| | | (SEQ ID NO: 177)(Kabat) | |
| | | CDR3: ITTVVNYAMDY (SEQ | |
| | | ID NO: 178) | |
| Murine | Humanized | EVQLVQSGAEVVKPGATVKL | EIVLTQSPATLSASPGERA |
| 1A1.A3- | 1A1.A3- | SCKASDFNIKDSYMHWVQQA | TLSCSTSSSVSYMHWFQQ |
| CEACAM5- | CEACAM5- | PGKGLEWIGWIDPENGDTEY | KPGQAPRLWIYSTSNLAS |
| B.02 | B.02- | APKFQGRATITADTSTDTAYL | GVPARFSGSGSGTDYTLTI |
| | BM- | ELSSLRSEDTAVYYCNVITTV | SSLEPEDFAVYYCQQRSN |
| | H1_NG | VNYAMDYWGQGTLVTVSS | YPLTFGAGTKVEIK (SEQ |
| | | (SEQ ID NO: 183) | ID NO: 184) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPENGD (SEQ ID | NO: 181) |
| | | NO: 176)(Chothia) or | CDR3: QQRSNYPLT (SEQ |
| | | WIDPENGDTEYAPKFQG | ID NO: 182) |
| | | (SEQ ID NO: 177)(Kabat) | |
| | | CDR3: ITTVVNYAMDY (SEQ | |
| | | ID NO: 178) | |
| Humanized | Humanized | EVQLVQSGAEVVKPGATVKL | EIVLTQSPATLSASPGERA |
| 1A1.A3- | 1A1.A3- | SCKASDFNIKDSYMHWVQQA | TLSCSTSSSVSYMHWFQQ |
| CEACAM5- | CEACAM5- | PGKGLEWIGWIDPESGDTEY | KPGQAPRLWIYSTSNLAS |
| B.02-BM | B.02- | APKFQGRATITADTSTDTAYL | GVPARFSGSGSGTDYTLTI |
| | BM- | ELSSLRSEDTAVYYCNVITTV | SSLEPEDFAVYYCQQRSN |
| | H1_SG | VNYAMDYWGQGTLVTVSS | YPLTFGAGTKVEIK (SEQ |
| | | (SEQ ID NO: 185) | ID NO: 184) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPESGD (SEQ ID | NO: 181) |
| | | NO: 186)(Chothia) or | CDR3: QQRSNYPLT (SEQ |
| | | WIDPESGDTEY APKFQG (SEQ | ID NO: 182) |
| | | ID NO: 187)(Kabat) | |
| | | CDR3: ITTVVNYAMDY (SEQ | |
| | | ID NO: 178) | |
| Humanized | Humanized | EVQLQQSGAEVVKPGATVKL | EIVLTQSPATLSASPGERA |
| 1A1.A3- | 1A1.A3- | SCKASDFNIKDSYMHWVTQA | TLSCSTSSSVSYMHWFQQ |
| CEACAM5- | CEACAM5- | PGKGLEWIGWIDPENGDTEY | KPGQAPRLWIYSTSNLAS |
| B.02-BM | B.02- | APKFQGRATMTADTSTDTAY | GVPARFSGSGSGTDYTLTI |
| | BM- | LELSSLRSEDTAVYYCNVITT | SSLEPEDFAVYYCQQRSN |
| | H2_NG | VVNYAMDYWGQGTLVTVSS | YPLTFGAGTKVEIK (SEQ |
| | | (SEQ ID NO: 188) | ID NO: 184) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPENGD (SEQ ID | NO: 181) |
| | | NO: 176)(Chothia) or | CDR3: QQRSNYPLT (SEQ |
| | | WIDPENGDTEYAPKFQG | ID NO: 182) |
| | | (SEQ ID NO: 177)(Kabat) | |
| | | CDR3: ITTVVNYAMDY (SEQ | |
| | | ID NO: 178) | |
| Humanized | Humanized | EVQLQQSGAEVVKPGATVKL | EIVLTQSPATLSASPGERA |
| 1A1.A3- | 1A1.A3- | SCKASDFNIKDSYMHWVTQA | TLSCSTSSSVSYMHWFQQ |
| CEACAM5- | CEACAM5- | PGKGLEWIGWIDPESGDTEY | KPGQAPRLWIYSTSNLAS |
| B.02-BM | B.02- | APKFQGRATMTADTSTDTAY | GVPARFSGSGSGTDYTLTI |
| | BM- | LELSSLRSEDTAVYYCNVITT | SSLEPEDFAVYYCQQRSN |
| | H2_SG | VVNYAMDYWGQGTLVTVSS | YPLTFGAGTKVEIK (SEQ |
| | | (SEQ ID NO: 189) | ID NO: 184) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPESGD (SEQ ID | NO: 181) |
| | | NO: 186)(Chothia) or | CDR3: QQRSNYPLT (SEQ |
| | | WIDPESGDTEY APKFQG (SEQ | ID NO: 182) |
| | | ID NO: 187)(Kabat) | |
| | | CDR3: ITTVVNYAMDY (SEQ | |
| | | ID NO: 178) | |
| Humanized | Humanized | EVQLQQSGAEVVKPGATVKL | EIVLTQSPATLSASPGERA |
| 1A1.A3- | 1A1.A3- | SCKASDFNIKDSYMHWVTQA | TLSCSTSSSVSYMHWFQQ |
| CEACAM5- | CEACAM5- | PGKGLEWIGWIDPEQGDTEY | KPGQAPRLWIYSTSNLAS |
| B.02-BM | B.02- | APKFQGRATMTADTSTDTAY | GVPARFSGSGSGTDYTLTI |
| | BM- | LELSSLRSEDTAVYYCNVITT | SSLEPEDFAVYYCQQRSN |
| | H2_QG | VVNYAMDYWGQGTLVTVSS | YPLTFGAGTKVEIK (SEQ |
| | | (SEQ ID NO: 190) | ID NO: 184) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPEQGD (SEQ ID | NO: 181) |
| | | NO: 191)(Chothia) or | CDR3: QQRSNYPLT (SEQ |
| | | WIDPEQGDTEYAPKFQG | ID NO: 182) |
| | | (SEQ ID NO: 192)(Kabat) | |
| | | CDR3: ITTVVNYAMDY (SEQ | |
| | | ID NO: 178) | |
| Humanized | VH | EVQLVQSGAEVVKPGATVKL | |
| 1A1.A3- | Humanized | SCKASDFNIKDAYMHWVQQ | |
| CEACAM5- | 1A1.A3- | APGKGLEWIGWIDPENGDTE | |
| B.02-BM | CEACAM5- | YAPKFQGRATITADTSTDTAY | |
| | B.02- | LELSSLRSEDTAVYYCNVITT | |
| | BM- | VVNYAMDYWGQGTLVTVSS | |
| | H1_(SH32A) | (SEQ ID NO: 356) | |
| | | CDR1: DFNIKDA (SEQ ID | |
| | | NO: 200)(Chothia) or DAYMH | |
| | | (SEQ ID NO: 201)(Kabat) | |
| | | CDR2: DPENGD (SEQ ID | |
| | | NO: 176)(Chothia) or | |
| | | WIDPENGDTEYAPKFQG | |
| | | (SEQ ID NO: 177)(Kabat) | |
| | | CDR3: ITTVVNYAMDY (SEQ | |
| | | ID NO: 178) | |
| Humanized | VH | EVQLVQSGAEVVKPGATVKL | |
| 1A1.A3- | Humanized | SCKASDFNIKDSYMHWVQQA | |
| CEACAM5- | 1A1.A3- | PGKGLEWIGWIDPEQGDTEY | |
| B.02-BM | CEACAM5- | APKFQGRATITADTSTDTAYL | |
| | B.02- | ELSSLRSEDTAVYYCNVITTV | |
| | BM- | VNYAMDYWGQGTLVTVSS | |
| | H1_(NH54Q) | (SEQ ID NO: 198) | |
| | | CDR1: DFNIKDS (SEQ ID | |
| | | NO: 174)(Chothia) or DSYMH | |
| | | (SEQ ID NO: 175)(Kabat) | |
| | | CDR2: DPEQGD (SEQ ID | |
| | | NO: 191)(Chothia) or | |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| Humanized | VH | EVQLVQSGAEVVKPGATVKL | |
| 1A1.A3- | Humanized | SCKASDFNIKDSYMHWVQQA | |
| CEACAM5- | 1A1.A3- | PGKGLEWIGWIDPESGDTEY | |
| B.02-BM | CEACAM5- | APKFQGRATITADTSTDTAYL | |
| | B.02- | ELSSLRSEDTAVYYCNVITTV | |
| | BM- | VNYAMDYWGQGTLVTVSS | |
| | H1_(NH54S) | (SEQ ID NO: 185) | |
| | | CDR1: DFNIKDS (SEQ ID | |
| | | NO: 174)(Chothia) or DSYMH | |
| | | (SEQ ID NO: 175)(Kabat) | |
| | | CDR2: DPESGD (SEQ ID | |
| | | NO: 186)(Chothia) or | |
| | | WIDPESGDTEYAPKFQG (SEQ | |
| | | ID NO: 187)(Kabat) | |
| | | CDR3: ITTVVNYAMDY (SEQ | |
| | | ID NO: 178) | |
| Humanized | Humanized | EVQLQQSGAEVVKPGATVKL | |
| 1A1.A3- | 1A1.A3- | SCKASDFNIKDSYMHWVTQA | |
| CEACAM5- | CEACAM5- | PGKGLEWIGWIDPEQGDTEY | |
| B.02-BM | B.02- | APKFQGRATMTADTSTDTAY | |
| | BM- | LELSSLRSEDTAVYYCNVITT | |
| | H2_(NH54Q) | VVNYAMDYWGQGTLVTVSS | |
| | | (SEQ ID NO: 190) | |
| | | CDR1: DFNIKDS (SEQ ID | |
| | | NO: 174)(Chothia) or DSYMH | |
| | | (SEQ ID NO: 175)(Kabat) | |
| | | CDR2: DPEQGD (SEQ ID | |
| | | NO: 191)(Chothia) or | |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| Humanized | Humanized | EVQLQQSGAEVVKPGATVKL | |
| 1A1.A3- | 1A1.A3- | SCKASDFNIKDSYMHWVTQA | |
| CEACAM5- | CEACAM5- | PGKGLEWIGWIDPESGDTEY | |
| B.02-BM | B.02- | APKFQGRATMTADTSTDTAY | |
| | BM- | LELSSLRSEDTAVYYCNVITT | |
| | H2_(NH54S) | VVNYAMDYWGQGTLVTVSS | |
| | | (SEQ ID NO: 189) | |
| | | CDR1: DFNIKDS (SEQ ID | |
| | | NO: 174)(Chothia) or DSYMH | |
| | | (SEQ ID NO: 175)(Kabat) | |
| | | CDR2: DPESGD (SEQ ID | |
| | | NO: 186)(Chothia) or | |
| | | WIDPESGDTEY APKFQG (SEQ | |
| | | ID NO: 187)(Kabat) | |
| | | CDR3: ITTVVNYAMDY (SEQ | |
| | | ID NO: 178) | |
| Humanized | Humanized | EVQLVQSGAEVVKPGATVKL | EIVLTQSPATLSASPGERA |
| 1A1.A3- | 1A1.A3- | SCKASDFNIKDSYMHWVQQA | TLSCSTSSSVSYMHWFQQ |
| CEACAM5- | CEACAM5- | PGKGLEWIGWIDPEQGDTEY | KPGQAPRLWIYSTSNLAS |
| B.02-BM | B.02- | APKFQGRATITADTSTDTAYL | GVPARFSGSGSGTDYTLTI |
| | BM- | ELSSLRSEDTAVYYCNVITTV | SSLEPEDFAVYYCQQRSN |
| | H1_QG- | VNYAMDYWGQGTLVTVSS | YPLTFGAGTKVEIK (SEQ |
| | VH | (SEQ ID NO: 198) | ID NO: 184) |
| | | CDR1: DFNIKDS (SEQ ID | CDR1: STSSSVSYMH (SEQ |
| | | NO: 174)(Chothia) or DSYMH | ID NO: 180) |
| | | (SEQ ID NO: 175)(Kabat) | CDR2: STSNLAS (SEQ ID |
| | | CDR2: DPEQGD (SEQ ID | NO: 181) |
| | | NO: 191)(Chothia) or | ID NO: 182) |
| | | WIDPEQGDTEYAPKFQG | |
| | | (SEQ ID NO: 192)(Kabat) | |
| | | CDR3: ITTVVNYAMDY (SEQ | |
| | | ID NO: 178) | |
| Humanized | Humanized | EVQLVQSGAEVVKPGATVKL | EIVLTQSPATLSASPGERA |
| 1A1.A3- | 1A1.A3- | SCKASDFNIKDAYMHWVQQ | TLSCSTSSSVSYMHWFQQ |
| CEACAM5- | CEACAM5- | APGKGLEWIGWIDPEQGDTE | KPGQAPRLWIYSTSNLAS |
| B.02-BM | B.02- | YAPKFQGRATITADTSTDTAY | GVPARFSGSGSGTDYTLTI |
| | BM- | LELSSLRSEDTAVYYCNVITT | SSLEPEDFAVYYCQQRSN |
| | H1_QG- | VVNYAMDYWGQGTLVTVSS | YPLTFGAGTKVEIK (SEQ |
| | VH | (SEQ ID NO: 199) | ID NO: 184) |
| | (Variant 2) | CDR1: DFNIKDA (SEQ ID | CDR1: STSSSVSYMH (SEQ |
| | | NO: 200)(Chothia) or DAYMH | ID NO: 180) |
| | | (SEQ ID NO: 201)(Kabat) | CDR2: STSNLAS (SEQ ID |
| | | CDR2: DPEQGD (SEQ ID | NO: 181) |
| | | NO: 191)(Chothia) or | CDR3: QQRSNYPLT (SEQ |
| | | WIDPEQGDTEYAPKFQG | ID NO: 182) |
| | | (SEQ ID NO: 192)(Kabat) | |
| | | CDR3: ITTVVNYAMDY (SEQ | |
| | | ID NO: 178) | |
| 4B10.B3- | 4B10.B3- | EVQLQESGAELMKPGASVKIS | |
| CEACAM5- | CEACAM5- | CKATGYTFSTYWIEWVKPRP | |
| A.02 | A.02_VH1 | GHGLEWIGEILPGTGTTNYNE | |
| | | KFKGKATFTADTSSNTAYMQ | |
| | | LSSLTSEDSAVYYCATLNGH | |
| | | GDYWYFDVWGAGTTVTVSS | |
| | | (SEQ ID NO: 639) | |
| | | CDR1: GYTFSTY (SEQ ID | |
| | | NO: 640)(Chothia) or TYWIE | |
| | | (SEQ ID NO: 641)(Kabat) | |
| | | CDR2: LPGTGT (SEQ ID | |
| | | NO: 642)(Chothia) or | |
| | | EILPGTGTTNYNEKFKG (SEQ | |
| | | ID NO: 643)(Kabat) | |
| | | CDR3: LNGHGDYWYFDV | |
| | | (SEQ ID NO: 644) | |
| 4B10.B3- | 4B10.B3- | QVQLQQSGAELMKPGASVKI | |
| CEACAM5- | CEACAM5- | SCKATGYTFSTYWIEWVKPR | |
| A.02 | A.02_VH2 | PGHGLEWIGEILPGTGTTNYN | |
| | | EKFKGKATFTADTSSNTAYM | |
| | | QLSSLTSEDSAVYYCATLNG | |
| | | HGDYWYFDVWGAGTTVTVS | |
| | | S (SEQ ID NO: 645) | |
| | | CDR1: GYTFSTY (SEQ ID | |
| | | NO: 640)(Chothia) or TYWIE | |
| | | (SEQ ID NO: 641)(Kabat) | |
| | | CDR2: LPGTGT (SEQ ID | |
| | | NO: 642)(Chothia) or | |
| | | EILPGTGTTNYNEKFKG (SEQ | |
| | | ID NO: 643)(Kabat) | |
| | | CDR3: LNGHGDYWYFDV | |
| | | (SEQ ID NO: 644) | |
| 4B10.B3- | 4B10.B3- | | QAVVTQESALTTSPGETV |
| CEACAM5- | CEACAM5- | | TLTCRSSTGGVTTSNYAN |
| A.02 | A.02_VL | | WVQEKPDHLFTGLIGGTN |
| | | | NRAPGVPARFSGSLIGDK |
| | | | AALTITGAQTEDEAIYFCA |
| | | | LWYSNHLVFGGGTKLTV |
| | | | L (SEQ ID NO: 646) |
| | | | CDR1: |
| | | | RSSTGGVTTSNYAN (SEQ |
| | | | ID NO: 647) |
| | | | CDR2: GTNNRAP (SEQ ID |
| | | | NO: 648) |
| | | | CDR3: ALWYSNHLV (SEQ |
| | | | ID NO: 649) |
| 13C7.A6- | 13C7.A6- | EVQLQESGPELVKPGASLKIS | |
| CEACAM5- | CEACAM5- | CKASGYSFTDYTMNWVKQS | |
| B.02 | B.02_VH1 | HGKNLEWIGLINPYNGGTTY | |
| | | NQKFKDMATLTVDKSSSTAY | |
| | | MELLSLTSEDSAVYYCARSE | |
| | | YGHSYWYFDVWGAGTTVTV | |
| | | SP (SEQ ID NO: 650) | |
| | | CDR1: GYSFTDY (SEQ ID | |
| | | NO: 651)(Chothia) or DYTMN | |
| | | (SEQ ID NO: 652)(Kabat) | |
| | | CDR2: NPYNGG (SEQ ID | |
| | | NO: 653)(Chothia) or | |
| | | LINPYNGGTTYNQKFKD (SEQ | |
| | | ID NO: 654)(Kabat) | |
| | | CDR3: SEYGHSYWYFDV | |
| | | (SEQ ID NO: 655) | |
| 13C7.A6- | 13C7.A6- | EVQLQQSGPELVKPGASLKIS | |
| CEACAM5- | CEACAM5- | CKASGYSFTDYTMNWVKQS | |
| B.02 | B.02_VH2 | HGKNLEWIGLINPYNGGTTY | |
| | | NQKFKDMATLTVDKSSSTAY | |
| | | MELLSLTSEDSAVYYCARSE | |
| | | YGHSYWYFDVWGAGTTVTV | |
| | | SS (SEQ ID NO: 656) | |
| | | CDR1: GYSFTDY (SEQ ID | |
| | | NO: 651)(Chothia) or DYTMN | |
| | | (SEQ ID NO: 652)(Kabat) | |
| | | CDR2: NPYNGG (SEQ ID | |
| | | NO: 653)(Chothia) or | |
| | | LINPYNGGTTYNQKFKD (SEQ | |
| | | ID NO: 654)(Kabat) | |
| | | CDR3: SEYGHSYWYFDV | |
| | | (SEQ ID NO: 655) | |
| 13C7.A6- | 13C7.A6- | | DIVMSQSPSSLAVSVGEK |
| CEACAM5- | CEACAM5- | | VTMSCKSSHSLLYGNFQN |
| B.02 | B.02_VL1 | | NYLAWYQQKPGQSPKLLI |
| | | | YWASTRESGVPDRFTGSG |
| | | | SGTDFTLTISSVKAEDLAV |
| | | | YYCQQYYSYPYTFGGGT |
| | | | KLEIK (SEQ ID NO: 106) |
| | | | CDR1: |
| | | | KSSHSLLYGNFQNNYLA |
| | | | (SEQ ID NO: 107) |
| | | | CDR2: WASTRES (SEQ ID |
| | | | NO: 108) |
| | | | CDR3: QQYYSYPYT (SEQ |
| | | | ID NO: 109) |
| 13C7.A6- | 13C7.A6- | | QIVLTQSPAIMSASPGEKV |
| CEACAM5- | CEACAM5- | | TLTCSPSSSVTYMHWYQQ |
| B.02 | B.02_VL2 | | KSGTSPKRWIYDTSKLAS |
| | | | GVPARFSGIGSGTSYSLTI |
| | | | NSMEAEDAATYYCQQWN |
| | | | NYPYTFGGGTRLEIK (SEQ |
| | | | ID NO: 657) |
| | | | CDR1: SPSSSVTYMH (SEQ |
| | | | ID NO: 658) |
| | | | CDR2: DTSKLAS (SEQ ID |
| | | | NO: 659) |
| | | | CDR3: QQWNNYPYT (SEQ |
| | | | ID NO: 660) |
| 13C7.A6- | 13C7.A6- | | DIQMTQSPASLSASVGDT |
| CEACAM5- | CEACAM5- | | VTITCRASENIYSYFAWY |
| B.02 | B.02_VL3 | | QQKQGKSPRLLVYNARA |
| | | | LAEGVPSRFSGSGSGTQFS |
| | | | KINSLQPEDFGSYYCQHL |
| | | | YGAPFTFGSGTKLEIK |
| | | | (SEQ ID NO: 661) |
| | | | CDR1: RASENIYSYFA |
| | | | (SEQ ID NO: 662) |
| | | | CDR2: NARALAE (SEQ ID |
| | | | NO: 663) |
| | | | CDR3: QHLYGAPFT (SEQ |
| | | | ID NO: 664) |
| 7E11.B2- | 7E11.B2- | QIQLVQSGPELKKPGETVKIS | |
| CEACAM5- | CEACAM5- | CKASGYSFTKYGMNWVKQA | |
| B.02 | B.02_VH1 | PGKGLKWMGWINTYSGEPTY | |
| | | ADDFEGRFAFSLETSANTAYL | |
| | | QINNLKNEDMATYFCARGGG | |
| | | FDYGFDYWGQGTTLTVST | |
| | | (SEQ ID NO: 665) | |
| | | CDR1: GYSFTKY (SEQ ID | |
| | | NO: 666)(Chothia) or KYGMN | |
| | | (SEQ ID NO: 667)(Kabat) | |
| | | CDR2: NTYSGE (SEQ ID | |
| | | NO: 668)(Chothia) or | |
| | | WINTYSGEPTY ADDFEG (SEQ | |
| | | ID NO: 669)(Kabat) | |
| | | CDR3: GGGFDYGFDY (SEQ | |
| | | ID NO: 670) | |
| 7E11.B2- | 7E11.B2- | QIQLVQSGPELKKPGETVKIS | |
| CEACAM5- | CEACAM5- | CKASGYSFTKYGMNWVKQA | |
| B.02 | B.02_VH2 | PGKGLKWMGWINTYSGEPTY | |
| | | ADDFEGRFAFSLETSANTAYL | |
| | | QINNLKNEDMATYFCARGGG | |
| | | FDYGFDYWGQGTTLTVSS | |
| | | (SEQ ID NO: 699) | |
| | | CDR1: GYSFTKY (SEQ ID | |
| | | NO: 666)(Chothia) or KYGMN | |
| | | (SEQ ID NO: 667)(Kabat) | |
| | | CDR2: NTYSGE (SEQ ID | |
| | | NO: 668)(Chothia) or | |
| | | WINTYSGEPTY ADDFEG (SEQ | |
| | | ID NO: 669)(Kabat) | |
| | | CDR3: GGGFDYGFDY (SEQ | |
| | | ID NO: 670) | |
| 7E11.B2- | 7E11.B2- | | QIVLTQSPAIMSASPGEKV |
| CEACAM5- | CEACAM5- | | TMTCSASSSVSYIHWYRQ |
| B.02 | B.02_VL | | RSGTSPKRWIYDTSKLAS |
| | | | GVPARFSGSGSGTSYSLTI |
| | | | STMEAEDAATYYCQQWS |
| | | | NYPYTFGGGTKLEIK (SEQ |
| | | | ID NO: 671) |
| | | | CDR1: SASSSVSYIH (SEQ |
| | | | ID NO: 672) |
| | | | CDR2: DTSKLAS (SEQ ID |
| | | | NO: 659) |
| | | | CDR3: QQWSNYPYT (SEQ |
| | | | ID NO: 673) |
| 10D6.E3- | 10D6.E3- | EVQLQQSGPELVKPGASMKIS | |
| CEACAM5- | CEACAM5- | CKASGYSFTDYTMNWVKQS | |
| B.02 | B.02 VH | HGKNLEWIGHIYPYNGGTTY | |
| | | NOKFQDKASLTADKSSSTAY | |
| | | MELLSLTSEDSAVYYCARGE | |
| | | FLRSYWYFDVWGAGTLVAV | |
| | | SS (SEQ ID NO: 674) | |
| | | CDR1: GYSFTDY (SEQ ID | |
| | | NO: 651)(Chothia) or DYTMN | |
| | | (SEQ ID NO: 652)(Kabat) | |
| | | CDR2: YPYNGG (SEQ ID | |
| | | NO: 675)(Chothia) or | |
| | | HIYPYNGGTTYNQKFQD | |
| | | (SEQ ID NO: 676)(Kabat) | |
| | | CDR3: GEFLRSYWYFDV (SEQ | |
| | | ID NO: 677) | |
| 10D6.E3- | 10D6.E3- | | QIVLTQSPAIMSASPGEKV |
| CEACAM5- | CEACAM5- | | TLTCSPSSSVTYMHWYQQ |
| B.02 | B.02_VL1 | | KSGTSPKRWIYDTSKLAS |
| | | | GVPARFSGIGSGTSYSLTI |
| | | | NSMEAEDAATYYCQQWN |
| | | | NYPYTFGGGTKLEIK (SEQ |
| | | | ID NO: 678) |
| | | | CDR1: SPSSSVTYMH (SEQ |
| | | | ID NO: 658) |
| | | | CDR2: DTSKLAS (SEQ ID |
| | | | NO: 659) |
| | | | CDR3: QQWNNYPYT (SEQ |
| | | | ID NO: 660) |
| 10D6.E3- | 10D6.E3- | | DIQMTQSPASLSASVGET |
| CEACAM5- | CEACAM5- | | VTITCRGSENIYSYLTWYQ |
| B.02 | B.02_VL2 | | QKQGKSPQLLVYNAKTL |
| | | | AEGVPSRFSGSGSGTQFSL |
| | | | KINSLQPEDFGRYYCQHL |
| | | | YSSPYTFGGGTKLEIK |
| | | | (SEQ ID NO: 679) |
| | | | CDR1: RGSENIYSYLT |
| | | | (SEQ ID NO: 680) |
| | | | CDR2: NAKTLAE (SEQ ID |
| | | | NO: 681) |
| | | | CDR3: QHLYSSPYT (SEQ |
| | | | ID NO: 682) |
| 13C7.F2- | 13C7.F2- | EVQLQQSGPELVKPGASLKIS | |
| CEACAM5- | CEACAM5- | CKASGYSFTDYTMNWVKQS | |
| B.02 | B.02_VH | HGKNLEWIGLINPYNGGTTY | |
| | | NQKFKDMATLTVDKSSSTAY | |
| | | MELLSLTSEDSAVYYCARSE | |
| | | YGHSYWYFDVWGAGTTVTV | |
| | | SS (SEQ ID NO: 656) | |
| | | CDR1: GYSFTDY (SEQ ID | |
| | | NO: 651)(Chothia) or DYTMN | |
| | | (SEQ ID NO: 652)(Kabat) | |
| | | CDR2: NPYNGG (SEQ ID | |
| | | NO: 653)(Chothia) or | |
| | | LINPYNGGTTYNQKFKD (SEQ | |
| | | ID NO: 654)(Kabat) | |
| | | CDR3: SEYGHSYWYFDV | |
| | | (SEQ ID NO: 655) | |
| 13C7.F2- | 13C7.F2- | | QIVLTQSPAIMSASPGEKV |
| CEACAM5- | CEACAM5- | | TLTCSPSSSVTYMHWYQQ |
| B.02 | B.02_VL1 | | KSGTSPKRWIYDTSKLAS |
| | | | GVPARFSGIGSGTSYSLTI |
| | | | NSMEAEDAATYYCQQWN |
| | | | NYPYTFGGGTRLEIK (SEQ |
| | | | ID NO: 657) |
| | | | CDR1: SPSSSVTYMH (SEQ |
| | | | ID NO: 658) |
| | | | CDR2: DTSKLAS (SEQ ID |
| | | | NO: 659) |
| | | | CDR3: QQWNNYPYT (SEQ |
| | | | ID NO: 660) |
| 13C7.F2- | 13C7.F2- | | DIQMTQSPASLSASVGDT |
| CEACAM5- | CEACAM5- | | VTITCRASENIYSYFAWY |
| B.02 | B.02_VL2 | | QQKQGKSPRLLVYNARA |
| | | | LAEGVPSRFSGSGSGTQFS |
| | | | LKINSLQPEDFGSYYCQHL |
| | | | YGAPFTFGSGTKLEIK |
| | | | (SEQ ID NO: 683) |
| | | | CDR1: RASENIYSYFA |
| | | | (SEQ ID NO: 662) |
| | | | CDR2: NARALAE (SEQ ID |
| | | | NO: 663) |
| | | | CDR3: QHLYGAPFT (SEQ |
| | | | ID NO: 664) |
| 16B11.G2- | 16B11.G2- | QVQLQESGPGLVTPSGTLSLT | EIVMTQSPATLSVSPGERA |
| CEACAM5- | CEACAM5- | CAVSGDSISSSHWWSWVRQP | TLSCRASQSVRSNLAWYL |
| B.01 | B.01_VH | PGKGLEWIGEIYHSGITNYRS | QKPGQAPRLLIYGASTRA |
| | and | SLKSRVTLSVDKSKNQFSLKL | TGIPARFSGSGSGTEFTLTI |
| | 16B11.G2- | TSVTAADTAVYYCARGGSGN | SSLQSEDFAVYYCQQYNN |
| | CEACAM5- | YEAFDIWGQGTLFTVSS (SEQ | WPLTFGGGTKVEIK (SEQ |
| | B.01_VL | ID NO: 684) | ID NO: 690) |
| | | CDR1: GDSISSSH (SEQ ID | CDR1: RASQSVRSNLA |
| | | NO: 685) | (SEQ ID NO: 62) |
| | | (Chothia) or SSHWWS (SEQ ID | CDR2: GASTRAT (SEQ ID |
| | | NO: 686)(Kabat) | NO: 63) |
| | | CDR2: YHSGI (SEQ ID NO: 687) | CDR3: QQYNNWPLT (SEQ |
| | | (Chothia) or | ID NO: 42) |
| | | EIYHSGITNYRSSLKS (SEQ ID | |
| | | NO: 688)(Kabat) | |
| | | CDR3: GGSGNYEAFDI (SEQ | |
| | | ID NO: 689) | |

Tier 3

| | | | |
|---|---|---|---|
| 1080_G01- | 1080_G01- | QVQLVESGGGVVQSGRSLRL | EIVLTQSPSTLSASVGDRV |
| CEACAM5 | CEACAM5 | SCAASGFMFSNFAMHWVRQ | TITCRASQSISNWLAWYQ |
| | | APGKGLEWVGVIWYDGSNK | QKPGKAPKLLIYKASSLES |
| | | FYADSVKGRFTISRDNSKNTL | GVPSRFSGSGSGTEFTLTIS |
| | | NLQMSRLRAEDTAVYYCAR | SLQPDDFATYYCQQYNSY |
| | | DGREVWRYYHYGMDVWGQ | SYTFGQGTKVEIK (SEQ ID |
| | | GTTVTVSS (SEQ ID NO: 377) | NO: 208) |
| | | CDR1: GFMFSNF (SEQ ID | CDR1: RASQSISNWLA |
| | | NO: 204)(Chothia) or NFAMH | (SEQ ID NO: 209) |
| | | (SEQ ID NO: 205)(Kabat) | CDR2: KASSLES (SEQ ID |
| | | CDR2: WYDGSN (SEQ ID | NO: 8) |
| | | NO: 70)(Chothia) or | CDR3: QQYNSYSYT (SEQ |
| | | VIWYDGSNKFYADSVKG | ID NO: 9) |
| | | (SEQ ID NO: 206)(Kabat) | |
| | | CDR3: | |
| | | DGREVWRYYHYGMDV (SEQ | |
| | | ID NO: 207) | |
| 1080_G01- | 1080_G01- | | EIVLTQSPSTLSASVGDRV |
| CEACAM5 | CEACAM5_VL | | TITCRASQSISNWLAWYQ |
| | Variant | | QKPGKAPKLLIYKASSLES |
| | | | GVPSRFSGSGSGTEFTLTIS |
| | | | SLQPDDFATYYCQQYNA |
| | | | YSYTFGQGTKVEIK (SEQ |
| | | | ID NO: 210) |
| | | | CDR1: RASQSISNWLA |
| | | | (SEQ ID NO: 209) |
| | | | CDR2: KASSLES (SEQ ID |
| | | | NO: 8) |
| | | | CDR3: QQYNAYSYT (SEQ |
| | | | ID NO: 27) |
| 1080_G01- | VH | EVQLVESGGGVVQSGRSLRL | |
| CEACAM5 | 1080_G01- | SCAASGFMFSNFAMHWVRQ | |
| | CEACAM5_ | APGKGLEWVGVIWYDGSNK | |
| | (QH1E) | FYADSVKGRFTISRDNSKNTL | |
| | | NLQMSRLRAEDTAVYYCAR | |
| | | DGREVWRYYHYGMDVWGQ | |
| | | GTTVTVSS (SEQ ID NO: 357) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 203) | |
| 1080_G01- | VH | QVQLVESGGGVVQSGRSLRL | |
| CEACAM5 | 1080_G01- | SCAASGFMFSNFAMHWVRQ | |
| | CEACAM5_ | APGKGLEWVGVIWYDASNK | |
| | (GH54A) | FYADSVKGRFTISRDNSKNTL | |
| | | NLQMSRLRAEDTAVYYCAR | |
| | | DGREVWRYYHYGMDVWGQ | |
| | | GTTVTVSS (SEQ ID NO: 358) | |
| | | CDR1: GFMFSNF (SEQ ID | |
| | | NO: 204)(Chothia) or NFAMH | |
| | | (SEQ ID NO: 205)(Kabat) | |
| | | CDR2: WYDASN (SEQ ID | |
| | | NO: 359)(Chothia) or | |
| | | VIWYDASNKFYADSVKG | |
| | | (SEQ ID NO: 360)(Kabat) | |
| | | CDR3: | |
| | | DGREVWRYYHYGMDV (SEQ | |
| | | ID NO: 203) | |
| 1080_G01- | VH | QVQLVESGGGVVQSGRSLRL | |
| CEACAM5 | 1080_G01- | SCAASGFMFSNFAMHWVRQ | |
| | CEACAM5_ | APGKGLEWVGVIWYDGSNK | |
| | (SH62A) | FYADAVKGRFTISRDNSKNTL | |
| | | NLQMSRLRAEDTAVYYCAR | |
| | | DGREVWRYYHYGMDVWGQ | |
| | | GTTVTVSS (SEQ ID NO: 361) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADAVKG | |
| | | (SEQ ID NO: 362)(Kabat) | |
| | | CDR3: | |
| | | DGREVWRYYHYGMDV (SEQ | |
| | | ID NO: 207) | |
| 1080_G01- | VH | QVQLVESGGGVVQSGRSLRL | |
| CEACAM5 | 1080_G01- | SCAASGFMFSNFAMHWVRQ | |
| | CEACAM5_ | APGKGLEWVGVIWYDGSNK | |
| | (NH73A) | FYADSVKGRFTISRDASKNTL | |
| | | NLQMSRLRAEDTAVYYCAR | |
| | | DGREVWRYYHYGMDVWGQ | |
| | | GTTVTVSS (SEQ ID NO: 363) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 207) | |
| 1080_G01- | VH | QVQLVESGGGVVQSGRSLRL | |
| CEACAM5 | 1080_G01- | SCAASGFMFSNFAMHWVRQ | |
| | CEACAM5_ | APGKGLEWVGVIWYDGSNK | |
| | (SH74A) | FYADSVKGRFTISRDNAKNTL | |
| | | NLQMSRLRAEDTAVYYCAR | |
| | | DGREVWRYYHYGMDVWGQ | |
| | | GTTVTVSS (SEQ ID NO: 364) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DGREVWRYYHYGMDV (SEQ | |
| | | ID NO: 207) | |
| 1080_G01- | VH | QVQLVESGGGVVQSGRSLRL | |
| CEACAM5 | 1080_G01- | SCAASGFMFSNFAMHWVRQ | |
| | CEACAM5_ | APGKGLEWVGVIWYDGSNK | |
| | (GH96A) | FYADSVKGRFTISRDNSKNTL | |
| | | NLQMSRLRAEDTAVYYCAR | |
| | | DAREVWRYYHYGMDVWGQ | |
| | | GTTVTVSS (SEQ ID NO: 365) | |
| | | CDR1: GFMFSNF (SEQ ID | |
| | | NO: 204)(Chothia) or NFAMH | |
| | | (SEQ ID NO: 205)(Kabat) | |
| | | CDR2: WYDGSN (SEQ ID | |
| | | NO: 70)(Chothia) or | |
| | | VIWYDGSNKFYADSVKG | |
| | | (SEQ ID NO: 206)(Kabat) | |
| | | CDR3: | |
| | | DAREVWRYYHYGMDV (SEQ | |
| | | ID NO: 366) | |
| 1078_C12- | 1078_C12- | QVQLVQSGAEVKKPGASVKV | DIQLTQSPSSLSASVGDRV |
| CEACAM5 | CEACAM5 | SCTASGYTFTGYFIHWVRQAP | TITCRASQSISSYLNWYQQ |
| | | GQGLEWMGWINPHSGATNY | KPGKAPKLLIYAASSLQSG |
| | | AQKFQGRVTMTRDTSISTAY | VPSRFSGSGSGTDFTLTISS |
| | | MELSSLRSDDTAVYYCARVS | LQPEDFATYYCQQSYGNP |
| | | YYGLDVWGQGTTVTVSS | LTFGGGTKVEIK (SEQ ID |
| | | (SEQ ID NO: 216) | NO: 222) |
| | | CDR1: GYTFTGY (SEQ ID | CDR1: RASQSISSYLN |
| | | NO: 217)(Chothia) or GYFIH | (SEQ ID NO: 223) |
| | | (SEQ ID NO: 218)(Kabat) | CDR2: AASSLQS (SEQ ID |
| | | CDR2: NPHSGA (SEQ ID | NO: 224) |
| | | NO: 219)(Chothia) or | CDR3: QQSYGNPLT (SEQ |
| | | WINPHSGATNYAQKFQG | ID NO: 225) |
| | | (SEQ ID NO: 220)(Kabat) | |
| | | CDR3: VSYYGLDV (SEQ ID | |
| | | NO: 221) | |
| 1078_C12- | VH 1078 | EVQLVQSGAEVKKPGASVKV | |
| CEACAM5 | C12- | SCTASGYTFTGYFIHWVRQAP | |
| | CEACAM5 | GQGLEWMGWINPHSGATNY | |
| | (QH1E) | AQKFQGRVTMTRDTSISTAY | |
| | | MELSSLRSDDTAVYYCARVS | |
| | | YYGLDVWGQGTTVTVSS | |
| | | (SEQ ID NO: 367) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 1078_C12- | VH 1078 | QVQLVQSGAEVKKPGASVKV | |
| CEACAM5 | C12- | SCTASGYTFTGYFIHWVRQAP | |
| | CEACAM5 | GQGLEWIGWINPHSGATNYA | |
| | (MH48I) | QKFQGRVTMTRDTSISTAYM | |
| | | ELSSLRSDDTAVYYCARVSY | |
| | | YGLDVWGQGTTVTVSS (SEQ | |
| | | ID NO: 368) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 1078_C12- | VH 1078 | QVQLVQSGAEVKKPGASVKV | |
| CEACAM5 | C12- | SCTASGYTFTGYFIHWVRQAP | |
| | CEACAM5 | GQGLEWVGWINPHSGATNY | |
| | (MH48V) | AQKFQGRVTMTRDTSISTAY | |
| | | MELSSLRSDDTAVYYCARVS | |
| | | YYGLDVWGQGTTVTVSS | |
| | | (SEQ ID NO: 369) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 1078_C12- | VH 1078 | QVQLVQSGAEVKKPGASVKV | |
| CEACAM5 | C12- | SCTASGYTFTGYFIHWVRQAP | |
| | CEACAM5 | GQGLEWMGWINPHSGATNY | |
| | (MH69I) | AQKFQGRVTITRDTSISTAYM | |
| | | ELSSLRSDDTAVYYCARVSY | |
| | | YGLDVWGQGTTVTVSS (SEQ | |
| | | ID NO: 370) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 1078_C12- | VH 1078 | QVQLVQSGAEVKKPGASVKV | |
| CEACAM5 | C12- | SCTASGYTFTGYFIHWVRQAP | |
| | CEACAM5 | GQGLEWMGWINPHSGATNY | |
| | (MH69V) | AQKFQGRVTVTRDTSISTAY | |
| | | MELSSLRSDDTAVYYCARVS | |
| | | YYGLDVWGQGTTVTVSS | |
| | | (SEQ ID NO: 371) | |
| | | CDR1: GYTFTGY (SEQ ID | |
| | | NO: 217)(Chothia) or GYFIH | |
| | | (SEQ ID NO: 218)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 1078_F02- | 1078_F02- | QVQLQESGAEVKKPGASVKV | DIVLTQTPSSLSASVGDRV |
| CEACAM5 | CEACAM5 | SCKASGYTFTGYYLHWVRQA | TITCWASQSISSYLNWYQ |
| | | PGQGLEWMGWINPNSGDTN | QKPGKAPKLLIYAASSLQS |
| | | YAQKFQGRVTMTRDTSISTA | GVPSRFSGSGSGTDFTLTI |
| | | YMELSRLRSDDTAVYYCARV | SSLQPEDFATYYCQQSYS |
| | | SYYGLDVWGQGTTVTVSS | TPLTFGGGTKLEIK (SEQ |
| | | (SEQ ID NO: 227) | ID NO: 231) |
| | | CDR1: GYTFTGY (SEQ ID | CDR1: WASQSISSYLN |
| | | NO: 217)(Chothia) or GYYLH | (SEQ ID NO: 232) |
| | | (SEQ ID NO: 228)(Kabat) | CDR2: AASSLQS (SEQ ID |
| | | CDR2: NPNSGD (SEQ ID | NO: 224) |
| | | NO: 229)(Chothia) or | CDR3: QQSYSTPLT (SEQ |
| | | WINPNSGDTNYAQKFQG | ID NO: 233) |
| | | (SEQ ID NO: 230)(Kabat) | |
| | | CDR3: VSYYGLDV (SEQ ID | |
| | | NO: 221) | |
| 1079_B08- | 1079_B08- | QVQLVQSGAEVKKPGASVKV | EIVLTQSPSSLSASVGDRV |
| CEACAM5 | CEACAM5 | SCKASGYTFTGYYLHWVRQA | TITCRASQSISSYLNWYQQ |
| | | PGQGLEWMGWINPSSGDTNY | KPGKAPKLLIYAASSLQSG |
| | | AQKFQGRVTMTRDTSISTAY | VPSRFSGSGSGTDFTLTISS |
| | | MELSRLRSDDTAVYYCARVS | LQPEDFATYYCQQSYSNP |
| | | YYGLDVWGQGTTVTVSS | LTFGGGTKVDIK (SEQ ID |
| | | (SEQ ID NO: 234) | NO: 237) |
| | | CDR1: GYTFTGY (SEQ ID | CDR1: RASQSISSYLN |
| | | NO: 217)(Chothia) or GYYLH | (SEQ ID NO: 223) |
| | | (SEQ ID NO: 228)(Kabat) | CDR2: AASSLQS (SEQ ID |
| | | CDR2: NPSSGD (SEQ ID | NO: 224) |
| | | NO: 235)(Chothia) or | CDR3: QQSYSNPLT (SEQ |
| | | WINPSSGDTNYAQKFQG | ID NO: 238) |
| | | (SEQ ID NO: 236)(Kabat) | |
| | | CDR3: VSYYGLDV (SEQ ID | |
| | | NO: 221) | |
| 1078_F02- | VH_1078_ | EVQLQESGAEVKKPGASVKV | |
| CEACAM5 | F02- | SCKASGYTFTGYYLHWVRQA | |
| | CEACAM5 | PGQGLEWMGWINPNSGDTN | |
| | (QH1E) | YAQKFQGRVTMTRDTSISTA | |
| | | YMELSRLRSDDTAVYYCARV | |
| | | SYYGLDVWGQGTTVTVSS | |
| | | (SEQ ID NO: 372) | |
| | | CDR1: GYTFTGY (SEQ ID | |
| | | NO: 217)(Chothia) or GYYLH | |
| | | (SEQ ID NO: 228)(Kabat) | |
| | | CDR2: NPHSGA (SEQ ID | |
| | | NO: 219)(Chothia) or | |
| | | WINPHSGATNYAQKFQG | |
| | | (SEQ ID NO: 220)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (N5H3S) | PGQGLEWMGWINPSSGDTNY | |
| | | AQKFQGRVTMTRDTSISTAY | |
| | | MELSRLRSDDTAVYYCARVS | |
| | | YYGLDVWGQGTTVTVSS | |
| | | (SEQ ID NO: 373) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (SH54A) | PGQGLEWMGWINPNAGDTN | |
| | | YAQKFQGRVTMTRDTSISTA | |
| | | YMELSRLRSDDTAVYYCARV | |
| | | SYYGLDVWGQGTTVTVSS | |
| | | (SEQ ID NO: 374) | |
| | | CDR1: GYTFTGY (SEQ ID | |
| | | NO: 217)(Chothia) or GYYLH | |
| | | (SEQ ID NO: 228)(Kabat) | |
| | | CDR2: NPNAGD (SEQ ID | |
| | | NO: 375)(Chothia) or | |
| | | WINPNAGDTNYAQKFQG | |
| | | (SEQ ID NO: 376)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (MH48I) | PGQGLEWIGWINPNSGDTNY | |
| | | AQKFQGRVTMTRDTSISTAY | |
| | | MELSRLRSDDTAVYYCARVS | |
| | | YYGLDVWGQGTTVTVSS | |
| | | (SEQ ID NO: 378) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (MH48V) | PGQGLEWVGWINPNSGDTNY | |
| | | AQKFQGRVTMTRDTSISTAY | |
| | | MELSRLRSDDTAVYYCARVS | |
| | | YYGLDVWGQGTTVTVSS | |
| | | (SEQ ID NO: 379) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (MH69I) | PGQGLEWMGWINPNSGDTN | |
| | | YAQKFQGRVTITRDTSISTAY | |
| | | MELSRLRSDDTAVYYCARVS | |
| | | YYGLDVWGQGTTVTVSS | |
| | | (SEQ ID NO: 380) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (MH69V) | PGQGLEWMGWINPNSGDTN | |
| | | YAQKFQGRVTVTRDTSISTAY | |
| | | MELSRLRSDDTAVYYCARVS | |
| | | YYGLDVWGQGTTVTVSS | |
| | | (SEQ ID NO: 381) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (MH80I) | PGQGLEWMGWINPNSGDTN | |
| | | YAQKFQGRVTMTRDTSISTA | |
| | | YIELSRLRSDDTAVYYCARVS | |
| | | YYGLDVWGQGTTVTVSS | |
| | | (SEQ ID NO: 382) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| 1078_F02- | VH1078_F02- | QVQLQESGAEVKKPGASVKV | |
| CEACAM5 | CEACAM5 | SCKASGYTFTGYYLHWVRQA | |
| | (MH80V) | PGQGLEWMGWINPNSGDTN | |
| | | YAQKFQGRVTMTRDTSISTA | |
| | | YVELSRLRSDDTAVYYCARV | |
| | | SYYGLDVWGQGTTVTVSS | |
| | | (SEQ ID NO: 383) | |
| | | CDR1: GYTFTGY (SEQ ID | |
| | | NO: 217)(Chothia) or GYYLH | |
| | | (SEQ ID NO: 228)(Kabat) | |
| | | CDR2: NPNSGD (SEQ ID | |
| | | NO: 229)(Chothia) or | |
| | | WINPNSGDTNYAQKFQG | |
| | | (SEQ ID NO: 230)(Kabat) | |
| | | CDR3: VSYYGLDV (SEQ ID | |
| | | NO: 221) | |
| 1079_B08- | VH | EVQLVQSGAEVKKPGASVKV | |
| CEACAM5 | 1079_B08- | SCKASGYTFTGYYLHWVRQA | |
| | CEACAM5 | PGQGLEWMGWINPSSGDTNY | |
| | (QH1E) | AQKFQGRVTMTRDTSISTAY | |
| | | MELSRLRSDDTAVYYCARVS | |
| | | YYGLDVWGQGTTVTVSS | |
| | | (SEQ ID NO: 384) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1079_B08- | VH | QVQLVQSGAEVKKPGASVKV | |
| CEACAM5 | 1079_B08- | SCKASGYTFTGYYLHWVRQA | |
| | CEACAM5 | PGQGLEWIGWINPSSGDTNY | |
| | (MH48I) | AQKFQGRVTMTRDTSISTAY | |
| | | MELSRLRSDDTAVYYCARVS | |
| | | YYGLDVWGQGTTVTVSS | |
| | | (SEQ ID NO: 385) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1079_B08- | VH | QVQLVQSGAEVKKPGASVKV | |
| CEACAM5 | 1079_B08- | SCKASGYTFTGYYLHWVRQA | |
| | CEACAM5 | PGQGLEWVGWINPSSGDTNY | |
| | (MH48V) | AQKFQGRVTMTRDTSISTAY | |
| | | MELSRLRSDDTAVYYCARVS | |
| | | YYGLDVWGQGTTVTVSS | |
| | | (SEQ ID NO: 386) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1079_B08- | VH | QVQLVQSGAEVKKPGASVKV | |
| CEACAM5 | 1079_B08- | SCKASGYTFTGYYLHWVRQA | |
| | CEACAM5 | PGQGLEWMGWINPSSGDTNY | |
| | (MH69I) | AQKFQGRVTITRDTSISTAYM | |
| | | ELSRLRSDDTAVYYCARVSY | |
| | | YGLDVWGQGTTVTVSS (SEQ | |
| | | ID NO: 387) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1079_B08- | VH | QVQLVQSGAEVKKPGASVKV | |
| CEACAM5 | 1079_B08- | SCKASGYTFTGYYLHWVRQA | |
| | CEACAM5 | PGQGLEWMGWINPSSGDTNY | |
| | (MH69V) | AQKFQGRVTVTRDTSISTAY | |
| | | MELSRLRSDDTAVYYCARVS | |
| | | YYGLDVWGQGTTVTVSS | |
| | | (SEQ ID NO: 388) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1079_B08- | VH | QVQLVQSGAEVKKPGASVKV | |
| CEACAM5 | 1079_B08- | SCKASGYTFTGYYLHWVRQA | |
| | CEACAM5 | PGQGLEWMGWINPSSGDTNY | |
| | (MH80I) | AQKFQGRVTMTRDTSISTAYI | |
| | | ELSRLRSDDTAVYYCARVSY | |
| | | YGLDVWGQGTTVTVSS (SEQ | |
| | | ID NO: 389) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1079_B08- | VH | QVQLVQSGAEVKKPGASVKV | |
| CEACAM5 | 1079_B08- | SCKASGYTFTGYYLHWVRQA | |
| | CEACAM5 | PGQGLEWMGWINPSSGDTNY | |
| | (MH80V) | AQKFQGRVTMTRDTSISTAY | |
| | | VELSRLRSDDTAVYYCARVS | |
| | | YYGLDVWGQGTTVTVSS | |
| | | (SEQ ID NO: 390) | |
| | | CDR1: GYTFTGY (SEQ ID | |
| | | NO: 217)(Chothia) or GYYLH | |
| | | (SEQ ID NO: 228)(Kabat) | |
| | | CDR2: NPSSGD (SEQ ID | |
| | | NO: 235)(Chothia) or | |
| | | WINPSSGDTNYAQKFQG | |
| | | (SEQ ID NO: 236)(Kabat) | |
| | | CDR3: VSYYGLDV (SEQ ID | |
| | | NO: 221) | |
| 1078_G03- | 1078_G03- | QVQLVESGGGLVQPGGSLRL | DIRMTQSPSTLSASVGDR |
| CEACAM5 | CEACAM5 | SCAASGFTFSSYAMSWVRQA | VTITCRASQSISSWLAWY |
| | | PGKGLEWVSEISGSGDRTSYA | QQKPGKAPKLLIYKASSL |
| | | DSVKGRFTISRDNSKNRLYLQ | ESGVPSRFSGSGSGTEFTL |
| | | MNRLRTEDTAVYYCAKDLGP | TISSLQPDDFATYYCQQY |
| | | SGWYGLFDYWGQGTLVTVS | NSYSTWTFGQGTKVDIK |
| | | S (SEQ ID NO: 160) | (SEQ ID NO: 164) |
| | | CDR1: GFTFSSY (SEQ ID | CDR1: RASQSISSWLA |
| | | NO: 2)(Chothia) or SYAMS | (SEQ ID NO: 165) |
| | | (SEQ ID NO: 3)(Kabat) | CDR2: KASSLES (SEQ ID |
| | | CDR2: SGSGDR (SEQ ID | NO: 8) |
| | | NO: 161)(Chothia) or | CDR3: QQYNSYSTWT |
| | | EISGSGDRTSYADSVKG (SEQ | (SEQ ID NO: 166) |
| | | ID NO: 162)(Kabat) | |
| | | CDR3: DLGPSGWYGLFDY | |
| | | (SEQ ID NO: 163) | |
| 1078_G03- | VH | EVQLVESGGGLVQPGGSLRL | |
| CEACAM5 | 1078_G03- | SCAASGFTFSSYAMSWVRQA | |
| | CEACAM5 | PGKGLEWVSEISGSGDRTSYA | |
| | (QH1E) | DSVKGRFTISRDNSKNRLYLQ | |
| | | MNRLRTEDTAVYYCAKDLGP | |
| | | SGWYGLFDYWGQGTLVTVS | |
| | | S (SEQ ID NO: 352) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| 1078_G03- | VH | QVQLVESGGGLVQPGGSLRL | |
| CEACAM5 | 1078_G03- | SCAASGFTFSSYAMSWVRQA | |
| | CEACAM5 | PGKGLEWVSEISGSGDRTSYA | |
| | (SH62A) | DAVKGRFTISRDNSKNRLYL | |
| | | QMNRLRTEDTAVYYCAKDL | |
| | | GPSGWYGLFDYWGQGTLVT | |
| | | VSS (SEQ ID NO: 353) | |
| | | CDR1: GFTFSSY (SEQ ID | |
| | | NO: 2)(Chothia) or SYAMS | |
| | | (SEQ ID NO: 3)(Kabat) | |
| | | CDR2: SGSGDR (SEQ ID | |
| | | NO: 161)(Chothia) or | |
| | | EISGSGDRTSYADSVKG (SEQ | |
| | | ID NO: 162)(Kabat) | |
| | | CDR3: DLGPSGWYGLFDY | |
| | | (SEQ ID NO: 163) | |
| 1079_A10- | 1079_A10- | QVQLVESGAEVKKPGASVKV | DIRMTQSPSSLSASVGDRV |
| CEACAM5 | CEACAM5 | SCKASGYTFTGYYLHWVRQA | TIICRASQSISSYLNWYQQ |
| | | PGQGLEWMGWINPNNGDTN | KPGKAPKLLIYAASSLQSG |
| | | YAQKFQGRVTMTRDTSISTA | VPSRFSGSGSGTDFTLTISS |
| | | YMELSRLRSDDTAVYYCARV | LQPEDFATYYCQQSYSNP |
| | | SYYGLDVWGQGTTVTVSS | LTFGGGTKVEIK (SEQ ID |
| | | (SEQ ID NO: 243) | NO: 246) |
| | | CDR1: GYTFTGY (SEQ ID | CDR1: RASQSISSYLN |
| | | NO: 217)(Chothia) or GYYLH | (SEQ ID NO: 223) |
| | | (SEQ ID NO: 228)(Kabat) | CDR2: AASSLQS (SEQ ID |
| | | CDR2: NPNNGD (SEQ ID | NO: 224) |
| | | NO: 244)(Chothia) or | CDR3: QQSYSNPLT (SEQ |
| | | WINPNNGDTNYAQKFQG | ID NO: 238) |
| | | (SEQ ID NO: 245)(Kabat) | |
| | | CDR3: VSYYGLDV (SEQ ID | |
| | | NO: 221) | |
| 1079_A10- | 1079_A10- | EVQLVESGAEVKKPGASVKV | DIRMTQSPSSLSASVGDRV |
| CEACAM5 | CEACAM5_ | SCKASGYTFTGYYLHWVRQA | TIICRASQSISSYLNWYQQ |
| | VH_ | PGQGLEWMGWINPNNGDTN | KPGKAPKLLIYAASSLQSG |
| | Variant | YAQKFQGRVTMTRDTSISTA | VPSRFSGSGSGTDFTLTISS |
| | | YMELSRLRSDDTAVYYCARV | LQPEDFATYYCQQSYSNP |
| | | SYYGLDVWGQGTTVTVSS | LTFGGGTKVEIK (SEQ ID |
| | | (SEQ ID NO: 247) | NO: 246) |
| | | CDR1: GYTFTGY (SEQ ID | CDR1: RASQSISSYLN |
| | | NO: 217)(Chothia) or GYYLH | (SEQ ID NO: 223) |
| | | (SEQ ID NO: 228)(Kabat) | CDR2: AASSLQS (SEQ ID |
| | | CDR2: NPNNGD (SEQ ID | NO: 224) |
| | | NO: 244)(Chothia) or | CDR3: QQSYSNPLT (SEQ |
| | | WINPNNGDTNYAQKFQG | ID NO: 238) |
| | | (SEQ ID NO: 245)(Kabat) | |
| | | CDR3: VSYYGLDV (SEQ ID | |
| | | NO: 221) | |
| 1079_A12- | 1079_A12- | QVQLVQSGGGLVQPGGSLRL | DIVLTQTPSSLSASVGDRV |
| CEACAM5 | CEACAM5 | SCAASGFTFSSYAMNWVRQA | TITCRASQSISSWLAWYQ |
| | | PGKGLEWVSGISGSGGSTKY | QKPGKAPKLLIYKASSLES |
| | | AESVKGRFTISRDNSKNTLYL | GVPSRFSGSGSGTEFTLTIS |
| | | QMNSLRADDTAVYYCAKDQ | SLQPDDFATYYCQQYNSY |
| | | DINVWYGLFPYWGQGTLVTV | SPLTFGGGTKVDIK (SEQ |
| | | SS (SEQ ID NO: 249) | ID NO: 255) |
| | | CDR1: GFTFSSY (SEQ ID | CDR1: RASQSISSWLA |
| | | NO: 2)(Chothia) or SYAMN | (SEQ ID NO: 165) |
| | | (SEQ ID NO: 250) | CDR2: KASSLES (SEQ ID |
| | | (Kabat) | NO: 8) |
| | | CDR2: SGSGGS (SEQ ID | CDR3: QQYNSYSPLT (SEQ |
| | | NO: 251)(Chothia) or | ID NO: 256) |
| | | GISGSGGSTKYAESVKG (SEQ | |
| | | ID NO: 253)(Kabat) | |
| | | CDR3: DQDINVWYGLFPY | |
| | | (SEQ ID NO: 254) | |
| 1079_A12- | 1079_A12- | EVQLVQSGGGLVQPGGSLRL | DIVLTQTPSSLSASVGDRV |
| CEACAM5 | CEACAM5_ | SCAASGFTFSSYAMNWVRQA | TITCRASQSISSWLAWYQ |
| | VH_ | PGKGLEWVSGISGSGGSTKY | QKPGKAPKLLIYKASSLES |
| | Variant | AESVKGRFTISRDNSKNTLYL | GVPSRFSGSGSGTEFTLTIS |
| | | QMNSLRADDTAVYYCAKDQ | SLQPDDFATYYCQQYNSY |
| | | DINVWYGLFPYWGQGTLVTV | SPLTFGGGTKVDIK (SEQ |
| | | SS (SEQ ID NO: 257) | ID NO: 255) |
| | | CDR1: GFTFSSY (SEQ ID | CDR1: RASQSISSWLA |
| | | NO: 2)(Chothia) or SYAMN | (SEQ ID NO: 165) |
| | | (SEQ ID NO: 250)(Kabat) | CDR2: KASSLES (SEQ ID |
| | | CDR2: SGSGGS (SEQ ID | NO: 8) |
| | | NO: 251)(Chothia) or | CDR3: QQYNSYSPLT (SEQ |
| | | GISGSGGSTKYAESVKG (SEQ | ID NO: 256) |
| | | ID NO: 253)(Kabat) | |
| | | CDR3: DQDINVWYGLFPY | |
| | | (SEQ ID NO: 254) | |
| 1078_C04- | 1078_C04- | EVQLVESGGGVVQPGRSLRL | EIVMTQSPSTLSASVGDRV |
| CEACAM5 | CEACAM5 | SCAASGFTFSSSGMHWVRQA | TITCRASQSISSWLAWYQ |
| | | PGKGLEWVAVIWYDGSNKY | QKPGKAPKLLIYKASSLES |
| | | YVDSVKGRFTISRDNSKNTLY | GVPSRFSGSGSGTDFTLTI |
| | | LQMNSLRAEDTAVYYCARD | SSLQPDDFATYYCQQYKS |
| | | GRQQLVQGSYYYGMDVWG | YSYTFGQGTKVEIK (SEQ |
| | | QGTTVTVSS (SEQ ID NO: 259) | ID NO: 263) |
| | | CDR1: GFTFSSS (SEQ ID | CDR1: RASQSISSWLA |
| | | NO: 260)(Chothia) or SSGMH | (SEQ ID NO: 165) |
| | | (SEQ ID NO: 261)(Kabat) | CDR2: KASSLES (SEQ ID |
| | | CDR2: WYDGSN (SEQ ID | NO: 8) |
| | | NO: 70)(Chothia) or | CDR3: QQYKSYSYT (SEQ |
| | | VIWYDGSNKYYVDSVKG | ID NO: 264) |
| | | (SEQ ID NO: 262)(Kabat) | |
| | | CDR3: | |
| | | DGRQQLVQGSYYYGMDV | |
| | | (SEQ ID NO: 265) | |
| 1080_F11- | 1080_F11- | QVQLVQSGGGLVQRGGSLRL | DIVMTQTPSTLSASVGDR |
| CEACAM5 | CEACAM5 | SCAASGFTFSSYAMSWVRQA | VTITCRASQSISSWLAWY |
| | | PGKGLERVSGISGSGGNTYYA | QQKPGKAPKLLIYKASSL |
| | | DSVKGRFTISRDNSKNTLYLQ | ESGVPSRFSGSGSGTEFTL |
| | | MNSLRADDTAVYYCARDQDI | TISSLQPDDFATYYCQQY |
| | | NVWYGLFAYWGQGTLVTVS | NSYFMYTFGQGTKVEIK |
| | | S (SEQ ID NO: 266) | (SEQ ID NO: 270) |
| | | CDR1: GFTFSSY (SEQ ID | CDR1: RASQSISSWLA |
| | | NO: 2)(Chothia) or SYAMS | (SEQ ID NO: 165) |
| | | (SEQ ID NO: 3)(Kabat) | CDR2: KASSLES (SEQ ID |
| | | CDR2: SGSGGN (SEQ ID | NO: 8) |
| | | NO: 267)(Chothia) or | CDR3: QQYNSYFMYT |
| | | GISGSGGNTYYADSVKG | (SEQ ID NO: 271) |
| | | (SEQ ID NO: 268)(Kabat) | |
| | | CDR3: DQDINVWYGLFAY | |
| | | (SEQ ID NO: 269) | |
| 1080_F11- | 1080_F11- | EVQLVQSGGGLVQRGGSLRL | DIVMTQTPSTLSASVGDR |
| CEACAM5 | CEACAM5_ | SCAASGFTFSSYAMSWVRQA | VTITCRASQSISSWLAWY |
| | VH | PGKGLERVSGISGSGGNTYYA | QQKPGKAPKLLIYKASSL |
| | Variant | DSVKGRFTISRDNSKNTLYLQ | ESGVPSRFSGSGSGTEFTL |
| | | MNSLRADDTAVYYCARDQDI | TISSLQPDDFATYYCQQY |
| | | NVWYGLFAYWGQGTLVTVS | NSYFMYTFGQGTKVEIK |
| | | S (SEQ ID NO: 272) | (SEQ ID NO: 270) |
| | | CDR1: GFTFSSY (SEQ ID | CDR1: RASQSISSWLA |
| | | NO: 2)(Chothia) or SYAMS | (SEQ ID NO: 165) |
| | | (SEQ ID NO: 3)(Kabat) | CDR2: KASSLES (SEQ ID |
| | | CDR2: SGSGGN (SEQ ID | NO: 8) |
| | | NO: 267)(Chothia) or | CDR3: QQYNSYFMYT |
| | | GISGSGGNTYYADSVKG | (SEQ ID NO: 271) |
| | | (SEQ ID NO: 268)(Kabat) | |
| | | CDR3: DQDINVWYGLFAY | |
| | | (SEQ ID NO: 269) | |
| 1081_E01- | 1081_E01- | QVQLQESGPGLVKPSETLSLT | DIVMTQTPSTLSASVGDR |
| CEACAM5 | CEACAM5 | CAVSGYSISSGYYWGWIRQPP | VTITCRASQSISSWLAWY |
| | | GKGLEWIGTIWHSGSTYDNPS | QQKPGKAPKLLIYKASSL |
| | | LKSRVTISVDTSKNQFSLKLSS | ESGVPSRFSGSGSGTDFTL |
| | | VTTADTAVYYCARADSSGW | TISSLQPEDFATYYCQQSY |
| | | YEDYWGQGTLVTVSS (SEQ | STPLTFGGGTKVEIK (SEQ |
| | | ID NO: 274) | ID NO: 280) |
| | | CDR1: GYSISSGY (SEQ ID | CDR1: RASQSISSWLA |
| | | NO: 275)(Chothia) or SGYYWG | (SEQ ID NO: 165) |
| | | (SEQ ID NO: 276)(Kabat) | CDR2: KASSLES (SEQ ID |
| | | CDR2: WHSGS (SEQ ID | NO: 8) |
| | | NO: 277)(Chothia) or | CDR3: QQSYSTPLT (SEQ |
| | | TIWHSGSTYDNPSLKS (SEQ | ID NO: 233) |
| | | ID NO: 278)(Kabat) | |
| | | CDR3: ADSSGWYEDY (SEQ | |
| | | ID NO: 279) | |
| 1081_E01- | 1081_E01- | EVQLQESGPGLVKPSETLSLT | DIVMTQTPSTLSASVGDR |
| CEACAM5 | CEACAM5_ | CAVSGYSISSGYYWGWIRQPP | VTITCRASQSISSWLAWY |
| | VH | GKGLEWIGTIWHSGSTYDNPS | QQKPGKAPKLLIYKASSL |
| | Variant | LKSRVTISVDTSKNQFSLKLSS | ESGVPSRFSGSGSGTDFTL |
| | | VTTADTAVYYCARADSSGW | TISSLQPEDFATYYCQQSY |
| | | YEDYWGQGTLVTVSS (SEQ | STPLTFGGGTKVEIK (SEQ |
| | | ID NO: 281) | ID NO: 280) |
| | | CDR1: GYSISSGY (SEQ ID | CDR1: RASQSISSWLA |
| | | NO: 275)(Chothia) or SGYYWG | (SEQ ID NO: 165) |
| | | (SEQ ID NO: 276)(Kabat) | CDR2: KASSLES (SEQ ID |
| | | CDR2: WHSGS (SEQ ID | NO: 8) |
| | | NO: 277)(Chothia) or | CDR3: QQSYSTPLT (SEQ |
| | | TIWHSGSTYDNPSLKS (SEQ | ID NO: 233) |
| | | ID NO: 278)(Kabat) | |
| | | CDR3: ADSSGWYEDY (SEQ | |
| | | ID NO: 279) | |
| 1083_A05- | 1083_A05- | QVQLVQSGGGLVQPGGSLRL | DIQVTQSPSTLSASVGDRV |
| CEACAM5 | CEACAM5 | SCAASGFIFSSYAMSWVRQA | TITCRASQSISSWLAWYQ |
| | | PGRGLEWVSGISGSGGSTNY | QKPGKAPKLLIYKASSLES |
| | | ADSVKGRFTISRDNSKNTLYL | GVPSRFSGSGSGTEFTLTIS |
| | | QMNSLRADDTAVYFCAKDL | SLQPDDFATYYCQQYNSY |
| | | DINVWYGLFDHWGQGTTVT | STWTFGQGTKLEIK (SEQ |
| | | VSS (SEQ ID NO: 283) | ID NO: 286) |
| | | CDR1: GFIFSSY (SEQ ID | CDR1: RASQSISSWLA |
| | | NO: 284)(Chothia) or SYAMS | (SEQ ID NO: 165) |
| | | (SEQ ID NO: 3)(Kabat) | CDR2: KASSLES (SEQ ID |
| | | CDR2: SGSGGS (SEQ ID | NO: 8) |
| | | NO: 251)(Chothia) or | CDR3: QQYNSYSTWT |
| | | GISGSGGSTNYADSVKG (SEQ | (SEQ ID NO: 166) |
| | | ID NO: 252)(Kabat) | |
| | | CDR3: DLDINVWYGLFDH | |
| | | (SEQ ID NO: 285) | |
| 1083_A05- | 1083_A05- | EVQLVQSGGGLVQPGGSLRL | DIQVTQSPSTLSASVGDRV |
| CEACAM5 | CEACAM5_ | SCAASGFIFSSYAMSWVRQA | TITCRASQSISSWLAWYQ |
| | VH | PGRGLEWVSGISGSGGSTNY | QKPGKAPKLLIYKASSLES |
| | Variant | ADSVKGRFTISRDNSKNTLYL | GVPSRFSGSGSGTEFTLTIS |
| | | QMNSLRADDTAVYFCAKDL | SLQPDDFATYYCQQYNSY |
| | | DINVWYGLFDHWGQGTTVT | STWTFGQGTKLEIK (SEQ |
| | | VSS (SEQ ID NO: 287) | ID NO: 286) |
| | | CDR1: GFIFSSY (SEQ ID | CDR1: RASQSISSWLA |
| | | NO: 284)(Chothia) or SYAMS | (SEQ ID NO: 165) |
| | | (SEQ ID NO: 3)(Kabat) | CDR2: KASSLES (SEQ ID |
| | | CDR2: SGSGGS (SEQ ID | NO: 8) |
| | | NO: 251)(Chothia) or | CDR3: QQYNSYSTWT |
| | | GISGSGGSTNYADSVKG (SEQ | (SEQ ID NO: 166) |
| | | ID NO: 252)(Kabat) | |
| | | CDR3: DLDINVWYGLFDH | |
| | | (SEQ ID NO: 285) | |
| 1085_D12- | 1085_D12- | EVQLVESGGGLVQPGGSLRL | DIRLTQSPSTLSASVGDRV |
| CEACAM5 | CEACAM5 | SCAASGFIFSSYAMNWVRQA | TITCRASQSISSWLAWYQ |
| | | PGRGLEWVSGISGSGGSTNY | QKPGKAPKLLIYKASSLES |
| | | ADSVKGRFTISRDNSKNTLYL | GVPSRFSGSGSGTEFTLTIS |
| | | QMNSLRADDTAVYFCARDQ | SMQPDDFATYYCQQYNS |
| | | DINVWYGLFVYWGQGTLVT | YSTWTFGQGTKVEIK |
| | | VSS (SEQ ID NO: 288) | (SEQ ID NO: 291) |
| | | CDR1: GFIFSSY (SEQ ID | CDR1: RASQSISSWLA |
| | | NO: 284)(Chothia) or SYAMN | (SEQ ID NO: 165) |
| | | (SEQ ID NO: 250)(Kabat) | CDR2: KASSLES (SEQ ID |
| | | CDR2: SGSGGS (SEQ ID | NO: 8) |
| | | NO: 251)(Chothia) or | CDR3: QQYNSYSTWT |
| | | GISGSGGSTNYADSVKG (SEQ | (SEQ ID NO: 166) |
| | | ID NO: 252)(Kabat) | |
| | | CDR3: DQDINVWYGLFVY | |
| | | (SEQ ID NO: 290) | |
| 1079_G12- | 1079_G12- | QVQLVQSGGGLVQPGGSLRL | DIVMTQTPSSLSASVGDR |
| CEACAM5 | CEACAM5 | SCAASGFTFSSYAMNWVRQA | VTITCRASQGISNYLAWY |
| | | PGKGLEWVSVISGNGGYTHY | QQKPGKVPKLLIYAASTL |
| | | ADSVKGRFTISRDNSKNTLYL | QSGVPSRFSGSGSGTDFTL |
| | | QMNSLRAEGTAVYYCAKDD | TISSLQPEDVATYYCQKY |
| | | WGGAFDIWGQGTTVTVSS | NSAPRTFGQGTKVEIK |
| | | (SEQ ID NO: 292) | (SEQ ID NO: 296) |
| | | CDR1: GFTFSSY (SEQ ID | CDR1: RASQGISNYLA |
| | | NO: 2)(Chothia) or SYAMN | (SEQ ID NO: 127) |
| | | (SEQ ID NO: 250)(Kabat) | CDR2: AASTLQS (SEQ ID |
| | | CDR2: SGNGGY (SEQ ID | NO: 128) |
| | | NO: 293)(Chothia) or | CDR3: QKYNSAPRT (SEQ |
| | | VISGNGGYTHYADSVKG | ID NO: 297) |
| | | (SEQ ID NO: 294)(Kabat) | |
| | | CDR3: DDWGGAFDI (SEQ ID | |
| | | NO: 295) | |
| 1079_G12- | 1079_G12- | EVQLVQSGGGLVQPGGSLRL | DIVMTQTPSSLSASVGDR |
| CEACAM5 | CEACAM5_ | SCAASGFTFSSYAMNWVRQA | VTITCRASQGISNYLAWY |
| | VH | PGKGLEWVSVISGNGGYTHY | QQKPGKVPKLLIYAASTL |
| | Variant | ADSVKGRFTISRDNSKNTLYL | QSGVPSRFSGSGSGTDFTL |
| | | QMNSLRAEGTAVYYCAKDD | TISSLQPEDVATYYCQKY |
| | | WGGAFDIWGQGTTVTVSS | NSAPRTFGQGTKVEIK |
| | | (SEQ ID NO: 298) | (SEQ ID NO: 296) |
| | | CDR1: GFTFSSY (SEQ ID | CDR1: RASQGISNYLA |
| | | NO: 2)(Chothia) or SYAMN | (SEQ ID NO: 127) |
| | | (SEQ ID NO: 250)(Kabat) | CDR2: AASTLQS (SEQ ID |
| | | CDR2: SGNGGY (SEQ ID | NO: 128) |
| | | NO: 293)(Chothia) or | CDR3: QKYNSAPRT (SEQ |
| | | VISGNGGYTHYADSVKG | ID NO: 297) |
| | | (SEQ ID NO: 294)(Kabat) | |
| | | CDR3: DDWGGAFDI (SEQ ID | |
| | | NO: 295) | |
| 1080_A01- | 1080_A01- | EVQLVQSGGGLVQPGGSVRL | DIRMTQSPSSLSASVGDRV |
| CEACAM5 | CEACAM5 | SCAASGFTFSSYAMNWVRQG | TITCRASQGISNYLAWYQ |
| | | PGKGLEWVSVISGNGGYTHY | QKPGKVPKLLIYAASTLQ |
| | | ADSVKGRFTISRDNSKNTLYL | SGVPSRFSGSGSGTDFTLT |
| | | QMNGLRADDTAVYYCAKDD | ISSLQPEDVATYYCQKYN |
| | | WGGAFDIWGQGTMVTVSS | SAPRTFGQGTKVDIK (SEQ |
| | | (SEQ ID NO: 299) | ID NO: 300) |
| | | CDR1: GFTFSSY (SEQ ID | CDR1: RASQGISNYLA |
| | | NO: 2)(Chothia) or SYAMN | (SEQ ID NO: 127) |
| | | (SEQ ID NO: 250)(Kabat) | CDR2: AASTLQS (SEQ ID |
| | | CDR2: SGNGGY (SEQ ID | NO: 128) |
| | | NO: 293)(Chothia) or | CDR3: QKYNSAPRT (SEQ |
| | | VISGNGGYTHYADSVKG | ID NO: 297) |
| | | (SEQ ID NO: 294)(Kabat) | |
| | | CDR3: DDWGGAFDI (SEQ ID | |
| | | NO: 295) | |
| 12C7.A2- | 12C7.A2- | QVQLVESGGGVVQPGRSLRL | DIQLTQSPSFLSASVGDRV |
| CEACAM5- | CEACAM5- | SCAASGFTFSNYAMHWVRQ | TITCRASQGISSYLAWYQ |
| B.01 | B.01 | APGKGLEWVTLIWYDGSKKY | QKPGKAPKLLIFPASTLQS |
| | | YADSVKGRFTISRDNSKNTLY | GVPSRFSGSGSGTEFTLTIS |
| | | LQMNSLRAEDTAVYYCARD | SLQPEDFATYYCQQLNFF |
| | | RAIPLIAPYFDYWGQGTLVTV | PPTFGPGTKVDIK (SEQ ID |
| | | SS (SEQ ID NO: 303) | NO: 309) |
| | | CDR1: GFTFSNY (SEQ ID | CDR1: RASQGISSYLA |
| | | NO: 304)(Chothia) or NYAMH | (SEQ ID NO: 131) |
| | | (SEQ ID NO: 305)(Kabat) | CDR2: PASTLQS (SEQ ID |
| | | CDR2: WYDGSK (SEQ ID | NO: 310) |
| | | NO: 306)(Chothia) or | CDR3: QQLNFFPPT (SEQ |
| | | LIWYDGSKKYYADSVKG | ID NO: 311) |
| | | (SEQ ID NO: 307)(Kabat) | |
| | | CDR3: DRAIPLIAPYFDY (SEQ | |
| | | ID NO: 308) | |
| 12C7.A2- | 12C7.A2- | EVQLVESGGGVVQPGRSLRL | DIQLTQSPSFLSASVGDRV |
| CEACAM5- | CEACAM5- | SCAASGFTFSNYAMHWVRQ | TITCRASQGISSYLAWYQ |
| B.01 | B.01_ | APGKGLEWVTLIWYDGSKKY | QKPGKAPKLLIFPASTLQS |
| | VH | YADSVKGRFTISRDNSKNTLY | GVPSRFSGSGSGTEFTLTIS |
| | Variant | LQMNSLRAEDTAVYYCARD | SLQPEDFATYYCQQLNFF |
| | | RAIPLIAPYFDYWGQGTLVTV | PPTFGPGTKVDIK (SEQ ID |
| | | SS (SEQ ID NO: 312) | NO: 309) |
| | | CDR1: GFTFSNY (SEQ ID | CDR1: RASQGISSYLA |
| | | NO: 304)(Chothia) or NYAMH | (SEQ ID NO: 131) |
| | | (SEQ ID NO: 305)(Kabat) | CDR2: PASTLQS (SEQ ID |
| | | CDR2: WYDGSK (SEQ ID | NO: 310) |
| | | NO: 306)(Chothia) or | CDR3: QQLNFFPPT (SEQ |
| | | LIWYDGSKKYYADSVKG | ID NO: 311) |
| | | (SEQ ID NO: 307)(Kabat) | |
| | | CDR3: DRAIPLIAPYFDY (SEQ | |
| | | ID NO: 308) | |
| 12C7.A2- | 12C7.A2- | QVQLQESGPGLVKPSETLSLT | DIQLTQSPSFLSASVGDRV |
| CEACAM5- | CEACAM5- | CTVSGGPIYSYYWSWIRQPPG | TITCRASQGISSYLAWYQ |
| B.01 | B.01_VH2 | KGLEWIGFIYSSGSTNYNPSL | QKPGKAPKLLIFPASTLQS |
| | | KSRVTISIDTSKSQFSLRLSSV | GVPSRFSGSGSGTEFTLTIS |
| | | TAADTAVYYCARGGDAFDI | SLQPEDFATYYCQQLNFF |
| | | WGQGTMVTVSS (SEQ ID | PPTFGPGTKVDIK (SEQ ID |
| | | NO: 691) | NO: 309) |
| | | CDR1: GGPIYSY (SEQ ID | CDR1: RASQGISSYLA |
| | | NO: 315)(Chothia) or SYYWS | (SEQ ID NO: 131) |
| | | (SEQ ID NO: 316)(Kabat) | CDR2: PASTLQS (SEQ ID |
| | | CDR2: YSSGS (SEQ ID | NO: 310) |
| | | NO: 317)(Chothia) or | CDR3: QQLNFFPPT (SEQ |
| | | FIYSSGSTNYNPSLKS (SEQ ID | ID NO: 311) |
| | | NO: 318)(Kabat) | |
| | | CDR3: GGDAFDI (SEQ ID | |
| | | NO: 319) | |
| 12A6.H2- | 12A6.H2- | QVQLVESGPGLVKPSETLSLT | DIQLTQSPSFLSASVGDRV |
| CEACAM5- | CEACAM5- | CTVSGGPIYSYYWSWIRQPPG | TITCRASQGISSYLAWYQ |
| B.01 | B.01 | KGLEWIGFIYSSGSTNYNPSL | QKPGKAPKLLIFPASTLQS |
| | | KSRVTISIDTSKSQFSLRLSSV | GVPSRFSGSGSGTEFTLTIS |
| | | TAADTAVYYCARGGDAFDI | SLQPEDFATYYCQQLNFF |
| | | WGQGTMVTVSS (SEQ ID | PPTFGPGTKVDIK (SEQ ID |
| | | NO: 314) | NO: 309) |
| | | CDR1: GGPIYSY (SEQ ID | CDR1: RASQGISSYLA |
| | | NO: 315)(Chothia) or SYYWS | (SEQ ID NO: 131) |
| | | (SEQ ID NO: 316)(Kabat) | CDR2: PASTLQS (SEQ ID |
| | | CDR2: YSSGS (SEQ ID | NO: 310) |
| | | NO: 317)(Chothia) or | CDR3: QQLNFFPPT (SEQ |
| | | FIYSSGSTNYNPSLKS (SEQ ID | ID NO: 311) |
| | | NO: 318)(Kabat) | |
| | | CDR3: GGDAFDI (SEQ ID | |
| | | NO: 319) | |
| 12A6.H2- | 12A6.H2- | EVQLVESGPGLVKPSETLSLT | DIQLTQSPSFLSASVGDRV |
| CEACAM5- | CEACAM5- | CTVSGGPIYSYYWSWIRQPPG | TITCRASQGISSYLAWYQ |
| B.01 | B.01_ | KGLEWIGFIYSSGSTNYNPSL | QKPGKAPKLLIFPASTLQS |
| | VH | KSRVTISIDTSKSQFSLRLSSV | GVPSRFSGSGSGTEFTLTIS |
| | Variant | TAADTAVYYCARGGDAFDI | SLQPEDFATYYCQQLNFF |
| | | WGQGTMVTVSS (SEQ ID | PPTFGPGTKVDIK (SEQ ID |
| | | NO: 320) | NO: 309) |
| | | CDR1: GGPIYSY (SEQ ID | CDR1: RASQGISSYLA |
| | | NO: 315)(Chothia) or SYYWS | (SEQ ID NO: 131) |
| | | (SEQ ID NO: 316)(Kabat) | CDR2: PASTLQS (SEQ ID |
| | | CDR2: YSSGS (SEQ ID | NO: 310) |
| | | NO: 317)(Chothia) or | CDR3: QQLNFFPPT (SEQ |
| | | FIYSSGSTNYNPSLKS (SEQ ID | ID NO: 311) |
| | | NO: 318)(Kabat) | |
| | | CDR3: GGDAFDI (SEQ ID | |
| | | NO: 319) | |
| 4G3.C3- | 4G3.C3- | QVQLQESGPGLVKPSETLSLT | DIQLTQSPSFLSASVGDRV |
| CEACAM5- | CEACAM5- | CTVSGGSLSSYYWSWIRQPPG | TITCRASQGTSSYLAWYQ |
| B.01 | B.01 | KGLEWLGYIYSSGSVNYNPSL | QKPGKAPKLLIYAVSTLQ |
| | | KSRVTMSIDTSQNQFSLKLSS | SGVPSRFSGSGSGTEFTLTI |
| | | VTAADTAVYYCARDADYFD | SSLQPTDFATYYCQQVIR |
| | | YWGQGTLVTVSS (SEQ ID | YPPTFGQGTKVEVK (SEQ |
| | | NO: 322) | ID NO: 326) |
| | | CDR1: GGSLSSY (SEQ ID | CDR1: RASQGTSSYLA |
| | | NO: 323)(Chothia) or SYYWS | (SEQ ID NO: 327) |
| | | (SEQ ID NO: 316)(Kabat) | CDR2: AVSTLQS (SEQ ID |
| | | CDR2: YSSGS (SEQ ID | NO: 328) |
| | | NO: 317)(Chothia) or | CDR3: QQVIRYPPT (SEQ |
| | | YIYSSGSVNYNPSLKS (SEQ | ID NO: 329) |
| | | ID NO: 324)(Kabat) | |
| | | CDR3: DADYFDY (SEQ ID | |
| | | NO: 325) | |
| 4G3.C3- | 4G3.C3- | EVQLQESGPGLVKPSETLSLT | DIQLTQSPSFLSASVGDRV |
| CEACAM5- | CEACAM5- | CTVSGGSLSSYYWSWIRQPPG | TITCRASQGTSSYLAWYQ |
| B.01 | B.01_ | KGLEWLGYIYSSGSVNYNPSL | QKPGKAPKLLIYAVSTLQ |
| | VH | KSRVTMSIDTSQNQFSLKLSS | SGVPSRFSGSGSGTEFTLTI |
| | Variant | VTAADTAVYYCARDADYFD | SSLQPTDFATYYCQQVIR |
| | | YWGQGTLVTVSS (SEQ ID | YPPTFGQGTKVEVK (SEQ |
| | | NO: 330) | ID NO: 326) |
| | | CDR1: GGSLSSY (SEQ ID | CDR1: RASQGTSSYLA |
| | | NO: 323)(Chothia) or SYYWS | (SEQ ID NO: 327) |
| | | (SEQ ID NO: 316)(Kabat) | CDR2: AVSTLQS (SEQ ID |
| | | CDR2: YSSGS (SEQ ID | NO: 328) |
| | | NO: 317)(Chothia) or | CDR3: QQVIRYPPT (SEQ |
| | | YIYSSGSVNYNPSLKS (SEQ | ID NO: 329) |
| | | ID NO: 324)(Kabat) | |
| | | CDR3: DADYFDY (SEQ ID | |
| | | NO: 325) | |
| 6D10.C8- | 6D10.C8- | QIQLVQSGPELKKPGETVKIS | QIVLTQSPAIMSASPGEKV |
| CEACAM5- | CEACAM5- | CKASGYTFTNYGMNWVKQA | TLTCSPSSSVTYMHWYQQ |
| B.02 | B.02_VH | PGKGLKWMGWINTYTGEPT | KSGTSPKRWIYDTSKLAS |
| | and | YTDDFKGRFAFSLETSASTAY | GVPARFSGIGSGTSYSLTI |
| | 6D10.C8- | LQITNLKNEDTATYFCARGD | NSMEAEDAATYYCQQWN |
| | CEACAM5- | GFDRGFAYWGQGTLVTVSA | NYPYTFGGGTRLEIT (SEQ |
| | B.02_VL | (SEQ ID NO: 692) | ID NO: 698) |
| | | CDR1: GYTFTNY (SEQ ID | CDR1: SPSSSVTYMH (SEQ |
| | | NO: 693)(Chothia) or NYGMN | ID NO: 658) |
| | | (SEQ ID NO: 694)(Kabat) | CDR2: DTSKLAS (SEQ ID |
| | | CDR2: NTYTGE (SEQ ID | NO: 659) |
| | | NO: 695)(Chothia) or | CDR3: QQWNNYPYT (SEQ |
| | | WINTYTGEPTYTDDFKG (SEQ | ID NO: 660) |
| | | ID NO: 696)(Kabat) | |
| | | CDR3: GDGFDRGFAY (SEQ ID | |
| | | NO: 697) | |

In certain embodiments, the second antigen-binding site that binds CEACAM5 (e.g., human CEACAM5, e.g., cynomolgus monkey CEACAM5) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 3, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Tables 3 or 4. In certain embodiments, the second antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antigen binding-site disclosed in Tables 3 or 4. In certain embodiments, the second antigen-binding site comprises the heavy chain CDR1, CDR2, and CDR3 and the light chain CDR1, CDR2, and CDR3 of an antigen binding-site disclosed in Table 3 or 4.

In certain embodiments, the second antigen-binding site is related to an scFv having a VH and VL in Table 3. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VH in Table 3. In certain embodiments, the second antigen-binding site comprises a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VL in Table 3. In certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VL in Table 3, and a VL that comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence of a VL in Table 3, wherein the VH and VL sequences are selected from a cognate pair of sequences listed in Table 3.

In certain embodiments, the VH comprises CDR1, CDR2, and CDR3 selected from a VH sequence listed in Table 3. In certain embodiments, the VL comprises CDR1, CDR2, and CDR3 selected from a VL sequence listed in Table 3. In certain embodiments, the second antigen-binding site comprises (a) a VH that comprises a CDR1, CDR2, and CDR3 selected from a VH sequence listed in Table 3; and (b) a VL that comprises CDR1, CDR2, and CDR3 selected from a VL sequence listed in Table 3, wherein the VH and VL sequences are selected from a cognate pair of sequences listed in Table 3.

In certain embodiments, the second antigen-binding site is related to an scFv having a VH and VL in Table 4. For example, in certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VH in Table 4. In certain embodiments, the second antigen-binding site comprises a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VL in Table 4. In certain embodiments, the second antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of a VH in Table 4, and a VL that comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence of a VL in Table 4, wherein the VH and VL in Table 4 are selected from the same clone listed in Table 4.

In certain embodiments, the VH comprises a CDR1, CDR2, and CDR3 of a VH sequence selected from Table 4. In certain embodiments, the VL comprises CDR1, CDR2, and CDR3 of a VL sequence selected from Table 4. In certain embodiments, the second antigen-binding site comprises (a) a VH that comprises a CDR1, CDR2, and CDR3 of a VH sequence selected from Table 4; and (b) a VL that comprises CDR1, CDR2, and CDR3 of a VL sequence selected from Table 4, wherein the VH and VL in Table 4 are selected from the same clone listed in Table 4.

In each of the foregoing embodiments, it is contemplated herein that the VH and/or VL sequences that together bind CEACAM5 may contain amino acid alterations (e.g., at least 1, 2, 3, 4, 5, or 10 amino acid substitutions, deletions, or additions) in the framework regions of the VH and/or VL without affecting their ability to bind to CEACAM5 significantly.

In certain embodiments, an antigen-binding site of the present invention that is derived from a cognate pair of Tier 1 in Table 3 or derived from a clone of Tier 1 in Table 4 binds a human CEACAM5 or a CEACAM5 variant or the extracellular region thereof at a K_{D} value less than or equal to 25 nM (e.g., less than or equal to 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM). It is understood that a smaller K_{D} value indicates a greater affinity. In certain embodiments, an antigen-binding site of the present invention that is derived from a cognate pair of Tier 2 in Table 3 or derived from a clone of Tier 2 in Table 4 binds a human CEACAM5 or a CEACAM5 variant or the extracellular region thereof at a K_{D} value greater than or equal to 15 nM (e.g., greater than or equal to 20 nM, 25 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, or 560 nM). In certain embodiments, an antigen-binding site of the present invention that is derived from a cognate pair of Tier 2 in Table 3 or derived from a clone of Tier 2 in Table 4 binds a human CEACAM5 or a CEACAM5 variant or the extracellular region thereof at a K_{D} value in the range of 15-560 nM, 15-400 nM, 15-300 nM, 15-200 nM, 15-100 nM, 15-80 nM, 20-560 nM, 20-400 nM, 20-300 nM, 20-200 nM, 20-100 nM, 20-80 nM, 25-100 nM, 25-560 nM, 25-400 nM, 25-300 nM, 25-200 nM, 25-100 nM, 25-80 nM, 50-560 nM, 50-400 nM, 50-300 nM, 50-200 nM, 50-100 nM, 50-80 nM, 100-560 nM, 100-400 nM, 100-300 nM, 100-200 nM, 120-560 nM, 120-400 nM, 120-300 nM, or 120-200 nM.

In certain embodiments, the antigen-binding site, as disclosed herein, comprises VH and VL sequences that are at least 90% (e.g., at least 91%, at least 92%, at least 93%, at least 94%, at least 95%, at least 96%, at least 97%, at least 98%, at least 99%, or 100%) identical to the VH and VL sequences, respectively, of a cognate pair of Tier 1 in Table 3 or a clone of Tier 1 in Table 4, and binds a human CEACAM5 or a CEACAM5 variant or the extracellular region thereof at a K_{D} value less than or equal to 25 nM (e.g., less than or equal to 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM). In certain embodiments, the antigen-binding site, as disclosed herein, comprises VH and VL sequences that are at least 90% (e.g., at least 91%, at least 92%, at least 93%, at least 94%, at least 95%, at least 96%, at least 97%, at least 98%, at least 99%, or 100%) identical to the VH and VL sequences, respectively, of a cognate pair of Tier 2 in Table 3 or a clone of Tier 2 in Table 4, and binds a human CEACAM5 or a CEACAM5 variant or the extracellular region thereof at a K_{D} value greater than or equal to 15 nM (e.g., greater than or equal to 20 nM, 25 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, or 560 nM. In certain embodiments, the antigen-binding site, as disclosed herein, comprises VH and VL sequences that are at least 90% (e.g., at least 91%, at least 92%, at least 93%, at least 94%, at least 95%, at least 96%, at least 97%, at least 98%, at least 99%, or 100%) identical to the VH and VL sequences, respectively, of a cognate pair of Tier 2 in Table 3 or a clone of Tier 2 in Table 4, and binds a human CEACAM5 or a CEACAM5 variant or the extracellular region thereof at a K_{D} value in the range of 15-560 nM, 15-400 nM, 15-300 nM, 15-200 nM, 15-100 nM, 15-80 nM, 20-560 nM, 20-400 nM, 20-300 nM, 20-200 nM, 20-100 nM, 20-80 nM, 25-100 nM, 25-560 nM, 25-400 nM, 25-300 nM, 25-200 nM, 25-100 nM, 25-80 nM, 50-560 nM, 50-400 nM, 50-300 nM, 50-200 nM, 50-100 nM, 50-80 nM, 100-560 nM, 100-400 nM, 100-300 nM, 100-200 nM, 120-560 nM, 120-400 nM, 120-300 nM, or 120-200 nM.

In certain embodiments, an antigen-binding site of the present invention that is derived from a cognate pair of Tier 1 or Tier 2 in Table 3 or a clone of Tier 1 or Tier 2 in Table 4 does not bind CEACAM1, CEACAM6, or CEACAM8 at a detectable level (e.g., as detected by surface plasmon resonance (SPR) or enzyme-linked immunosorbent assay (ELISA) for in vitro binding, or by flow cytometry for binding to cells expressing the respective antigen). In certain embodiments, an antigen-binding site of the present invention that is derived from a cognate pair of Tier 3 in Table 3 or a clone of Tier 3 in Table 4 binds CEACAM1, CEACAM6, or CEACAM8 at a detectable level (e.g., with a K_{D} value greater than or equal to 100 nM, 200 nM, 500 nM, 1 μM, 2 μM, 5 μM, or 10 μM).

In certain embodiments, the antigen-binding site, as disclosed herein, comprises VH and VL sequences that are at least 90% (e.g., at least 91%, at least 92%, at least 93%, at least 94%, at least 95%, at least 96%, at least 97%, at least 98%, at least 99%, or 100%) identical to the VH and VL sequences, respectively, of a cognate pair of Tier 1 or Tier 2 in Table 3 or a clone of Tier 1 or Tier 2 in Table 4, and does not bind CEACAM1, CEACAM6, or CEACAM8 at a detectable level (e.g., as detected by surface plasmon resonance (SPR) or enzyme-linked immunosorbent assay (ELISA) for in vitro binding, or by flow cytometry for binding to cells expressing the respective antigen). In certain embodiments, the antigen-binding site, as disclosed herein, comprises VH and VL sequences that are at least 90% (e.g., at least 91%, at least 92%, at least 93%, at least 94%, at least 95%, at least 96%, at least 97%, at least 98%, at least 99%, or 100%) identical to the VH and VL sequences, respectively, of a cognate pair of Tier 3 in Table 3 or a clone of Tier 3 in Table 4, and binds CEACAM1, CEACAM6, or CEACAM8 at a detectable level (e.g., with a K_{D} value greater than or equal to 100 nM, 200 nM, 500 nM, 1 μM, 2 μM, 5 μM, or 10 μM).

In certain embodiments of any one of the antigen-binding sites disclosed herein, where the N-terminal amino acid of a heavy chain variable region is a Gln (Q), the Q can be replaced by Glu (E), thereby generating a variant called “QE.” In certain embodiments of any one of the antigen-binding sites disclosed herein, where the N-terminal amino acid of a heavy chain variable region is a Gln (Q) or Glu (E), the Q or E can be replaced by pyroglutamate (pE). An antigen-binding site generated from such replacement falls within the same tier as the parental antigen-binding site.

In certain embodiments, the second antigen-binding site competes for binding to CEACAM5 (e.g., human CEACAM5, e.g., cynomolgus CEACAM5) with an antigen-binding site described above. In certain embodiments, the antigen-binding site of the present invention competes with an antigen-binding site related to a clone selected from Table 2, wherein the clones are selected from the group consisting of. Clone PH_420-CEACAM5, Clone 1078_C04-CEACAM5, Clone 1079_H05-CEACAM5, 7A10.A7-CEACAM5-B.01, 8H2.B10-CEACAM5-B.01, Murine 16F6.A2-CEACAM5-B.02, Humanized 16F6.A2-CEACAM5-B.02-BM, PH_415-CEACAM5, PH_416-CEACAM5, PH_418-CEACAM5, PH_419-CEACAM5, PH_417-CEACAM5, PH_421-CEACAM5, 1078_G03-CEACAM5, Murine 1A1.A3-CEACAM5-B.02, Humanized 1A1.A3-CEACAM5-B.02-BM, 1080_G01-CEACAM5, 1078_C12-CEACAM5, 1078_F02-CEACAM5, 1079_B08-CEACAM5, 1078_G03-CEACAM5, 1079_A10-CEACAM5, 1079_A12-CEACAM5, 1078_C04-CEACAM5, 1080_F11-CEACAM5, 1081_E01-CEACAM5, 1083_A05-CEACAM5, 1085_D12-CEACAM5, 1079_G12-CEACAM5, 1080_A01-CEACAM5, 12C7.A2-CEACAM5-B.01, 12A6.H2-CEACAM5-B.01, 4G3.C3-CEACAM5-B.01, 4B10.B3-CEACAM5-A.02, 13C7.A6-CEACAM5-B.02, 7E11.B2-CEACAM5-B.02, 10D6.E3-CEACAM5-B.02, 13C7.F2-CEACAM5-B.02, 16B11.G2-CEACAM5-B.01, and 6D10.C8-CEACAM5-B.02. In some embodiments, the antigen-binding site of the present invention competes with an antigen-binding site comprising a VL sequence and VH sequence selected from Table 1, wherein the VH sequence and VL sequence are from a cognate pair in Table 1 or a clone in Table 2. In some embodiments, the antigen-binding site of the present invention competes with an antigen-binding site comprising a CDRH1, CDRH2, CDRH3, CDRL1, CDRL2, and CDRL3, wherein the CDRs are selected from a cognate pair listed in Table 1 or a clone listed in Table 2.

### Fc Domain

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Accordingly, in certain embodiments, the antibody Fc domain or the portion thereof comprises a hinge and a CH2 domain. Based on the known domains, mutations can be selected to enhance or reduce the binding affinity to CD16, such as by using phage-displayed libraries or yeast surface-displayed cDNA libraries, or can be designed based on the known three-dimensional structure of the interaction. The binding of CD16 to an Fc domain can be determined by routine methods, e.g., by surface plasmon resonance (SPR) or enzyme-linked immunosorbent assay (ELISA) for in vitro binding, or by flow cytometry for binding to cells expressing CD16 on their cell surfaces.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitution(s) (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as a human IgG1 constant region, an IgG2 constant region, IgG3 constant region, or IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence DKTHTCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTISK AKGQPREPQVYTLPPSRDELTKNQVSLTCLVKGFYPSDIAVEWESNGQPENNYKTTPPV LDSDGSFFLYSKLTVDKSRWQQGNVFSCSVMHEALHNHYTQKSLSLSPG (SEQ ID NO:531). In some other embodiments, the amino acid sequence of the constant region is at least 90% identical to an antibody constant region from another mammal, such as rabbit, dog, cat, mouse, or horse.

In some embodiments, the antibody constant domain linked to the scFv or the Fab fragment is able to bind to CD16. In some embodiments, the protein incorporates a portion of an antibody Fc domain (for example, a portion of an antibody Fc domain sufficient to bind CD16), wherein the antibody Fc domain comprises a hinge and a CH2 domain (for example, a hinge and a CH2 domain of a human IgG1 antibody), and/or amino acid sequences at least 90% identical to amino acid sequence 234-332 of a human IgG antibody.

One or more mutations can be incorporated into the constant region as compared to human IgG1 constant region, for example at Q347, Y349, L351, 5354, E356, E357, K360, Q362, S364, T366, L368, K370, N390, K392, T394, D399, 5400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T3661, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, K409R, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cx of a human IgG1 constant region may be at amino acid E123, F116, S176, V163, S174, and/or T164.

Alternatively, amino acid substitutions could be selected from the following sets of substitutions shown in Table 5.

TABLE 5

| | First | Second |
|---|---|---|
| | Polypeptide | Polypeptide |
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 6.

TABLE 6

| | First | Second |
|---|---|---|
| | Polypeptide | Polypeptide |
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 7.

TABLE 7

| | First | Second |
|---|---|---|
| | Polypeptide | Polypeptide |
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 8.

TABLE 8

| First | Second |
|---|---|
| Polypeptide | Polypeptide |
| L351Y, D399R, D399K, S400K, | T366V, T366I, T366L, T366M, |
| S400R, Y407A, Y4071, Y407V | N390D, N390E, K392L, |
| | K392M, K392V, K392F |
| | K392D, K392E, K409F, |
| | K409W, T411D and T411E |

Alternatively, at least one amino acid substitution could be selected from the following sets of substitutions in Table 9, where the position(s) indicated in the First Polypeptide column is replaced by any known negatively-charged amino acid, and the position(s) indicated in the Second Polypeptide Column is replaced by any known positively-charged amino acid.

TABLE 9

| First | Second |
|---|---|
| Polypeptide | Polypeptide |
| K392, K370, K409, or K439 | D399, E356, or E357 |

Alternatively, at least one amino acid substitution could be selected from the following set in Table 10, where the position(s) indicated in the First Polypeptide column is replaced by any known positively-charged amino acid, and the position(s) indicated in the Second Polypeptide Column is replaced by any known negatively-charged amino acid.

TABLE 10

| First | Second |
|---|---|
| Polypeptide | Polypeptide |
| D399, E356, or E357 | K409, K439, K370, or K392 |

Alternatively, amino acid substitutions could be selected from the following sets in Table 11.

TABLE 11

| First | Second |
|---|---|
| Polypeptide | Polypeptide |
| T350V, L351Y, F405A, | T350V, T366L, K392L, |
| and Y407V | and T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of: T366, L368 and Y407.

In some embodiments, the amino acid sequence of one polypeptide chain of the antibody constant region differs from the amino acid sequence of an IgG1 constant region at one or more positions selected from the group consisting of: T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of: E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of: Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of: Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of: E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of: L351, D399, S400 and Y407 and wherein the amino acid sequence of the other polypeptide chain of the antibody constant region differs from the amino acid sequence of an IgG1 constant region at one or more positions selected from the group consisting of: T366, N390, K392, K409 and T411.

In some embodiments, the amino acid sequence of one polypeptide chain of the antibody constant region differs from the amino acid sequence of an IgG1 constant region at one or more positions selected from the group consisting of: T366, N390, K392, K409 and T411 and wherein the amino acid sequence of the other polypeptide chain of the antibody constant region differs from the amino acid sequence of an IgG1 constant region at one or more positions selected from the group consisting of: L351, D399, S400 and Y407.

In some embodiments, the amino acid sequence of one polypeptide chain of the antibody constant region differs from the amino acid sequence of an IgG1 constant region at one or more positions selected from the group consisting of: Q347, Y349, K360, and K409, and wherein the amino acid sequence of the other polypeptide chain of the antibody constant region differs from the amino acid sequence of an IgG1 constant region at one or more positions selected from the group consisting of: Q347, E357, D399 and F405.

In some embodiments, the amino acid sequence of one polypeptide chain of the antibody constant region differs from the amino acid sequence of an IgG1 constant region at one or more positions selected from the group consisting of: Q347, E357, D399 and F405, and wherein the amino acid sequence of the other polypeptide chain of the antibody constant region differs from the amino acid sequence of an IgG1 constant region at one or more positions selected from the group consisting of: Y349, K360, Q347 and K409.

In some embodiments, the amino acid sequence of one polypeptide chain of the antibody constant region differs from the amino acid sequence of an IgG1 constant region at one or more positions selected from the group consisting of: K370, K392, K409 and K439, and wherein the amino acid sequence of the other polypeptide chain of the antibody constant region differs from the amino acid sequence of an IgG1 constant region at one or more positions selected from the group consisting of: D356, E357 and D399.

In some embodiments, the amino acid sequence of one polypeptide chain of the antibody constant region differs from the amino acid sequence of an IgG1 constant region at one or more positions selected from the group consisting of: D356, E357 and D399, and wherein the amino acid sequence of the other polypeptide chain of the antibody constant region differs from the amino acid sequence of an IgG1 constant region at one or more positions selected from the group consisting of: K370, K392, K409 and K439.

In some embodiments, the amino acid sequence of one polypeptide chain of the antibody constant region differs from the amino acid sequence of an IgG1 constant region at one or more positions selected from the group consisting of: L351, E356, T366 and D399, and wherein the amino acid sequence of the other polypeptide chain of the antibody constant region differs from the amino acid sequence of an IgG1 constant region at one or more positions selected from the group consisting of: Y349, L351, L368, K392 and K409.

In some embodiments, the amino acid sequence of one polypeptide chain of the antibody constant region differs from the amino acid sequence of an IgG1 constant region at one or more positions selected from the group consisting of: Y349, L351, L368, K392 and K409, and wherein the amino acid sequence of the other polypeptide chain of the antibody constant region differs from the amino acid sequence of an IgG1 constant region at one or more positions selected from the group consisting of: L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

### Exemplary Multi-Specific Binding Proteins

Listed below are examples of TriNKETs comprising an antigen-binding site that binds CEACAM5 and an antigen-binding site that binds NKG2D each linked to an antibody constant region, wherein the antibody constant regions include mutations that enable heterodimerization of two Fc chains. The CDR sequences under Chothia are bold and the CDR sequences under Kabat are underlined.

TriNKETs are contemplated in the F3 format, i.e., the antigen-binding site that binds CEACAM5 is a Fab, and the antigen-binding site that binds NKG2D is an scFv. All the TriNKETs shown infra are in the F3′ format, i.e., the antigen-binding site that binds CEACAM5 is an scFv and the antigen-binding site that binds NKG2D is an Fab. In each TriNKET, the scFv comprises substitution of Cys in the VH and VL regions, facilitating formation of a disulfide bridge between the VH and VL of the scFv.

The VH and VL of the scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins of the present invention, and resist cleavage from proteases. For example, glycine and serine residues generally provide protease resistance. In certain embodiments, the VL is linked N-terminal or C-terminal to the VH via a (GlyGlyGlyGlySer)₄ ((G₄S)₄) linker (SEQ ID NO:532).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO:533), (GGS)ₙ (SEQ ID NO:534), (GGGS)ₙ (SEQ ID NO:535), (GGSG)ₙ (SEQ ID NO:536), (GGSGG)ₙ (SEQ ID NO:537), and (GGGGS)ₙ (SEQ ID NO:538) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from the group consisting of: SEQ ID NO:532, SEQ ID NO:539, SEQ ID NO:540, SEQ ID NO:541, SEQ ID NO:542, SEQ ID NO:543, SEQ ID NO:544, SEQ ID NO:545, SEQ ID NO:546, and SEQ ID NO:547, as listed in Table 12.

TABLE 12

| SEQ ID | Amino Acid Sequence |
|---|---|
| SEQ ID | GSGSGSGSGSGSGSGSGSGS |
| NO: 539 | |
| SEQ ID | GGSGGSGGSGGSGGSGGSGGSGGSGGSGGS |
| NO: 540 | |
| SEQ ID | GGGSGGGSGGGSGGGSGGGSGGGSGGGSG |
| NO: 541 | GGSGGGSGGGS |
| SEQ ID | GGSGGGSGGGSGGGSGGGSGGGSGGGSGG |
| NO: 542 | GSGGGSGGGSG |
| SEQ ID | GGSGGGGSGGGGSGGGGSGGGGSGGGGSG |
| NO: 543 | GGGSGGGGSGGGGSGGGGSGG |
| SEQ ID | GGGGSGGGGSGGGGSGGGGSGGGGSGGGG |
| NO: 544 | SGGGGSGGGGSGGGGSGGGGS |
| SEQ ID | GGGGSGGGGSGGGGSGGGGS |
| NO: 532 | |
| SEQ ID | GGGGSGGGGSGGGGS |
| NO: 545 | |
| SEQ ID | GGGGSGGGGSGGGGSGGGGSGGGGSGGGG |
| NO: 546 | SGGGGSGGGGSGGGGSGGGGSGGGGSGGG |
| | GSGGGGSGGGGSGGGGSGGGGSGGGGSGG |
| | GGSGGGGSGGGGS |
| SEQ ID | GGSGGGGSGGGGSGGGGSGGGGSGGGGSG |
| NO: 547 | GGGSGGGGSGGGGSGGGGSGGGGSGGGGS |
| | GGGGSGGGGSGGGGSGGGGSGGGGSGGGG |
| | SGGGGSGGGGSGG |

In the F3′-TriNKETs, the CEACAM5-binding scFv is linked to the N-terminus of an Fc via a Gly-Ser linker. The Ala-Ser or Gly-Ser linker is included at the elbow hinge region sequence to balance between flexibility and optimal geometry. In certain embodiments, an additional sequence Thr-Lys-Gly can be added N-terminal or C-terminal to the Ala-Ser or Gly-Ser sequence at the hinge.

As used herein to describe these exemplary TriNKETs, an Fc includes an antibody hinge, CH2, and CH3. In each exemplary TriNKET, the Fc domain linked to an scFv comprises the mutations of Q347R, D399V, and F405T, and the Fc domain linked to a Fab comprises matching mutations K360E and K409W for forming a heterodimer. The Fc domain linked to the scFv further includes an S354C substitution in the CH3 domain, which forms a disulfide bond with a Y349C substitution on the Fc linked to the Fab. These substitutions are bold in the sequences described in this subsection.

For example, a TriNKET of the present disclosure is F3′-GB1. F3′-GB1 includes (a) a CEACAM5-binding scFv sequence derived from the VH and VL sequences in Cognate Pair A1 of Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB1 includes three polypeptides: GB1-VL-VH-Fc, A49MI-VH-CH1-Fc, and A49MI-VL-CL.

GB1-VL-VH-Fc
(SEQ ID NO: 548)
DIRMTQSPSTLSASVGDRVTITCWASQSISSWLAWYQQKPGKAPKLLIYKASSLESGVPS
RFSGSGSGTEFTLTISSLQPDDFATYYCQQYNSYSYTFGCGTKLEIK
GGGGSGGGGSGGGGSGGGGS
QVQLVQSGGGLVQPGGSLRLSCAASGFTFSSYAMSWVRQAPGKCLEWVSAIFNSGGST
YYADSVKGRFTVSRDNSKNTLYLQMNSLRAEDTALYYCAKDLGGYNYGLFDYWGQGT
LVTVSS
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP APIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG
A49MI-VH-CHI-Fc
(SEQ ID NO: 549)
EVQLVESGGGLVKPGGSLRLSCAASGFTFSSYSMNWVRQAPGKGLEWVSSISSSSSYIYY
ADSVKGRFTISRDNAKNSLYLQMNSLRAEDTAVYYCARGAPIGAAAGWFDPWGQGTL
VTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVCTLPPSR
DELTENQVSLTCLVKGFYPSDIAVEWESNGQPENNYKTTPPVLDSDGSFFLYSWLTVDK
SRWQQGNVFSCSVMHEALHNHYTQKSLSLSPG
A49MI-VL-CL
(SEQ ID NO: 550)
DIQMTQSPSSVSASVGDRVTITCRASQGISSWLAWYQQKPGKAPKLLIYAASSLQSGVPS
RFSGSGSGTDFTLTISSLQPEDFATYYCQQGVSFPRTFGGGTKVEIK
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

GB1-VL-VH-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described below. The scFv includes a heavy chain variable domain of GB1 connected to the C-terminus of a light chain variable domain of GB1 via a (G₄S)₄ linker (SEQ ID NO: 532). The heavy and the light variable domains of the scFv are also connected through a disulfide bridge formed from the cysteine heterodimerization mutations, indicated in bold-underlining in the sequence above.

A49MI-VH-CH1-Fc represents the heavy chain portion of the Fab fragment, which comprises a heavy chain variable domain (SEQ ID NO:508) of NKG2D-binding A49MI and a CH1 domain, connected to an Fc domain. The Fc domain in A49MI-VH-CH1-Fc includes a Y349C substitution in the CH3 domain, which forms a disulfide bond with an S354C substitution on the Fc in GB1-VL-VH-Fc. In A49MI-VH-CH1-Fc, the Fc domain also includes K360E and K409W substitutions for heterodimerization with the Fc in GB1-VL-VH-Fc.

A49MI-VL-CL represents the light chain portion of the Fab fragment comprising a light chain variable domain of NKG2D-binding A49MI (SEQ ID NO:493) and a light chain constant domain.

Another TriNKET of the present disclosure is F3′-GB3. F3′-GB3 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB3 includes three polypeptides: GB3-VH-VL-Fc, A49MI-VH-CH1-Fc, and A49MI-VL-CL. A49MI-VH-CH1-Fc and A49MI-VL-CL are described above in the context of F3′GB1. The polypeptide of GB3-VH-VL-Fc is set forth below.

GB3-VH-VL-Fc
(SEQ ID NO: 551)
EVQLVQSGGGLVQPGGSLRLSCAASGFTFSSYAMSWVRQAPGKCLEWVSAIFNSGGST
YYADSVKGRFTVSRDNSKNTLYLQMNSLRAEDTALYYCAKDLGGYNYGLFDYWGQGT
LVTVSS
GGGGSGGGGSGGGGSGGGGS
DIRMTQSPSTLSASVGDRVTITCWASQSISSWLAWYQQKPGKAPKLLIYKASSLESGVPS
RFSGSGSGTEFTLTISSLQPDDFATYYCQQYNSYSYTFGCGTKLEIK
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB3-VH-VL-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB3 connected to the C-terminus of a heavy chain variable domain of GB3 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB5. F3′-GB5 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB5 includes three polypeptides: GB5-VH-VL-Fc, A49MI-VH-CH1-Fc, and A49MI-VL-CL. A49MI-VH-CH1-Fc and A49MI-VL-CL are described above in the context of F3′GB1. The polypeptide of GB5-VH-VL-Fc is set forth below.

GB5-VH-VL-Fc
(SEQ ID NO: 552)
QVQLVQSGGGLVQPGGSQRLSCAASGFTFTSYAMSWVRQAPGKCLEWVSAISGTGDST
FYADSVKGRFTFSRDNSKNTLYLQMNSLRAEDTAVYYCAKDLGWLQYGLFDYWGQGT
LVTVSS
GGGGSGGGGSGGGGSGGGGS
DIQLTQSPATLSVSPGERATLSCRASQSVSSSYLAWYQQKPGQAPRLLIYGASSRATGIPD
RFSGSGSGTDFTLTISRLEPEDFAVYYCQQYNNWPLTFGCGTKVEIK
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB5-VH-VL-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB5 connected to the C-terminus of a heavy chain variable domain of GB5 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB7. F3′-GB7 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB7 includes three polypeptides: GB7-VH-VL-Fc, A49MI-VH-CH1-Fc, and A49MI-VL-CL. A49MI-VH-CH1-Fc and A49MI-VL-CL are described above in the context of F3′GB1. The polypeptide of GB7-VH-VL-Fc is set forth below.

GB7-VH-VL-Fc
(SEQ ID NO: 553)
EVQLVQSGGGLVQPGGSQRLSCAASGFTFTSYAMSWVRQAPGKCLEWVSAISGTGDST
FYADSVKGRFTFSRDNSKNTLYLQMNSLRAEDTAVYYCAKDLGWLQYGLFDYWGQGT
LVTVSS
GGGGSGGGGSGGGGSGGGGS
DIQLTQSPATLSVSPGERATLSCRASQSVSSSYLAWYQQKPGQAPRLLIYGASSRATGIPD
RFSGSGSGTDFTLTISRLEPEDFAVYYCQQYNNWPLTFGCGTKVEIK
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB7-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB7 connected to the C-terminus of a heavy chain variable domain of GB7 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB9. F3′-GB9 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB9 includes three polypeptides: GB9-VL-VH-Fc, A49MI-VH-CH1-Fc, and A49MI-VL-CL. A49MI-VH-CH1-Fc and A49MI-VL-CL are described above in the context of F3′GB1. The polypeptide of GB9-VL-VH-Fc is set forth below.

GB9-VL-VH-Fc
(SEQ ID NO: 554)
DIQLTQSPATLSVSPGERATLSCRASQSVSSSYLAWYQQKPGQAPRLLIYGASSRATGIPD
RFSGSGSGTDFTLTISRLEPEDFAVYYCQQYNNWPLTFGCGTKVEIK
GGGGSGGGGSGGGGSGGGGS
QVQLVQSGGGLVQPGGSQRLSCAASGFTFTSYAMSWVRQAPGKCLEWVSAISGTGDST
FYADSVKGRFTFSRDNSKNTLYLQMNSLRAEDTAVYYCAKDLGWLQYGLFDYWGQGT
LVTVSS
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB9-VL-VH-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB9 connected to the N-terminus of a heavy chain variable domain of GB9 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB11. F3′-GB11 includes (a) a CEACAM5-binding scFv sequence derived from the VH and VL sequences in the corresponding Cognate Pair in Table 2 linked to an Fe domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB11 includes three polypeptides: GB11-VH-VL-Fc, A49MI-VH-CH1-Fc, and A49MI-VL-CL. A49MI-VH-CH1-Fc and A49MI-VL-CL are described above in the context of F3′GB1. The polypeptide of GB11-VH-VL-Fc is set forth below.

GB11-VH-VL-Fc
(SEQ ID NO: 555)
QVQLQESGPGLVRPSGTLSLTCAVSGGSISSPTWWSWVRQPPGKCLEWIGEIHPSGRTNY
NPSLKSRVTISVDKSKNQFSLKLGSVTAADTAVYYCAREGFYYGSGNYYYFDYWGQGT
LVTVSS
GGGGSGGGGSGGGGSGGGGS
DIVMTQTPATLSASVGDRVTITCRASQSVRSNLAWYQQKPGQAPRLLIYGASTRATGIPA
RFSGSGSGTEFTLTISSLQSEDFAVYYCQQYNNWPTFGCGTRLEIK
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB11-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB11 connected to the C-terminus of a heavy chain variable domain of GB11 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB13. F3′-GB13 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB13 includes three polypeptides: GB13-VH-VL-Fc, A49MI-VH-CH1-Fc, and A49MI-VL-CL. A49MI-VH-CH1-Fc and A49MI-VL-CL are described above in the context of F3′GB1. The polypeptide of GB13-VH-VL-Fc is set forth below.

GB13-VH-VL-Fc
(SEQ ID NO: 556)
EVQLQESGPGLVRPSGTLSLTCAVSGGSISSPTWWSWVRQPPGKCLEWIGEIHPSGRTNY
NPSLKSRVTISVDKSKNQFSLKLGSVTAADTAVYYCAREGFYYGSGNYYYFDYWGQGT
LVTVSS
GGGGSGGGGSGGGGSGGGGS
DIVMTQTPATLSASVGDRVTITCRASQSVRSNLAWYQQKPGQAPRLLIYGASTRATGIPA
RFSGSGSGTEFTLTISSLQSEDFAVYYCQQYNNWPTFGCGTRLEIK
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB13-VH-VL-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB13 connected to the C-terminus of a heavy chain variable domain of GB13 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB15. F3′-GB15 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB15 includes three polypeptides: GB15-VL-VH-Fc, A49MI-VH-CH1-Fc, and A49MI-VL-CL. A49MI-VH-CH1-Fc and A49MI-VL-CL are described above in the context of F3′GB1. The polypeptide of GB15-VL-VH-Fc is set forth below.

GB15-VL-VH-Fc
(SEQ ID NO: 557)
EVQLQESGPGLVRPSGTLSLTCAVSGGSISSPTWWSWVRQPPGKCLEWIGEIHPSGRTNY
NPSLKSRVTISVDKSKNQFSLKLGSVTAADTAVYYCAREGFYYGSGNYYYFDYWGQGT
LVTVSS
GGGGSGGGGSGGGGSGGGGS
DIVMTQTPATLSASVGDRVTITCRASQSVRSNLAWYQQKPGQAPRLLIYGASTRATGIPA
RFSGSGSGTEFTLTISSLQSEDFAVYYCQQYNNWPTFGCGTRLEIK
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB15-VL-VH-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB15 connected to the N-terminus of a heavy chain variable domain of GB15 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB17. F3′-GB17 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB17 includes three polypeptides: GB17-VH-VL-Fc, A49MI-VH-CH1-Fc, and A49MI-VL-CL. A49MI-VH-CH1-Fc and A49MI-VL-CL are described above in the context of F3′GB1. The polypeptide of GB17-VH-VL-Fc is set forth below.

GB17-VH-VL-Fc
(SEQ ID NO: 558)
QVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSNGYNYLDWYLQKPGQSPQLLISLGSIRASG
VPDRFSGSGSGTNFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB17-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB17 connected to the C-terminus of a heavy chain variable domain of GB17 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB19. F3′-GB19 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB19 includes three polypeptides: GB19-VH-VL-Fc, A49MI-VH-CH1-Fc, and A49MI-VL-CL. A49MI-VH-CH1-Fc and A49MI-VL-CL are described above in the context of F3′GB1. The polypeptide of GB19-VH-VL-Fc is set forth below.

GB19-VH-VL-Fc
(SEQ ID NO: 559)
QVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSNGYNYLDWYLQKPGQSPQLLISLGSIRASG
VPDRFSGSGSGTDFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB19-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB19 connected to the C-terminus of a heavy chain variable domain of GB19 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB21. F3′-GB21 includes (a) a CEACAM5-binding scFv sequence derived from the VH and VL sequences in the corresponding Cognate Pair in Table 2 linked to an Fe domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB21 includes three polypeptides: GB21-VH-VL-Fc, A49MI-VH-CH1-Fc, and A49MI-VL-CL. A49MI-VH-CH1-Fc and A49MI-VL-CL are described above in the context of F3′GB1. The polypeptide of GB21-VH-VL-Fc is set forth below.

GB21-VH-VL-Fc
(SEQ ID NO: 560)
QVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSNGYNYLDWYLQKPGQSPQLLISLGSIRASG
VPDRFSGSGSGTQFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB21-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB21 connected to the C-terminus of a heavy chain variable domain of GB21 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB23. F3′-GB23 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB23 includes three polypeptides: GB23-VH-VL-Fc, A49MI-VH-CH1-Fc, and A49MI-VL-CL. A49MI-VH-CH1-Fc and A49MI-VL-CL are described above in the context of F3′GB1. The polypeptide of GB23-VH-VL-Fc is set forth below.

GB23-VH-VL-Fc
(SEQ ID NO: 561)
QVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSNGYNYLDWYLQKPGQSPQLLISLGSIRASG
VPDRFSGSGSGTNFALTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB23-VH-VL-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB23 connected to the C-terminus of a heavy chain variable domain of GB23 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB25. F3′-GB25 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB25 includes three polypeptides: GB25-VH-VL-Fc, A49MI-VH-CH1-Fc, and A49MI-VL-CL. A49MI-VH-CH1-Fc and A49MI-VL-CL are described above in the context of F3′GB1. The polypeptide of GB25-VH-VL-Fc is set forth below.

GB25-VH-VL-Fc
(SEQ ID NO: 562)
EVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSNGYNYLDWYLQKPGQSPQLLISLGSIRASG
VPDRFSGSGSGTDFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB25-VH-VL-Fc represents the full sequence of a CEACAM5-binding scFv linked to an Fe domain via a hinge comprising Gly-Ser. The Fe domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB25 connected to the C-terminus of a heavy chain variable domain of GB5 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB27. F3′-GB27 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB27 includes three polypeptides: GB27-VH-VL-Fc, A49MI-VH-CH1-Fc, and A49MI-VL-CL. A49MI-VH-CH1-Fc and A49MI-VL-CL are described above in the context of F3′GB1. The polypeptide of GB27-VH-VL-Fc is set forth below.

GB27-VH-VL-Fc
(SEQ ID NO: 563)
EVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSSGYNYLDWYLQKPGQSPQLLISLGSIRASG
VPDRFSGSGSGTDFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB27-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB27 connected to the C-terminus of a heavy chain variable domain of GB27 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB29. F3′-GB29 includes (a) a CEACAM5-binding scFv sequence derived from the VH and VL sequences in the corresponding Cognate Pair in Table 2 linked to an Fc domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB29 includes three polypeptides: GB29-VH-VL-Fc, A49MI-VH-CH1-Fc, and A49MI-VL-CL. A49MI-VH-CH1-Fc and A49MI-VL-CL are described above in the context of F3′GB1. The polypeptide of GB29-VH-VL-Fc is set forth below.

GB29-VH-VL-Fc
(SEQ ID NO: 564)
EVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSQGYNYLDWYLQKPGQSPQLLISLGSIRASG
VPDRFSGSGSGTDFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB29-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB29 connected to the C-terminus of a heavy chain variable domain of GB29 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

Another TriNKET of the present disclosure is F3′-GB31. F3′-GB31 includes (a) a CEACAM5-binding scFv sequence derived from the VH and VL sequences in the corresponding Cognate Pair in Table 2 linked to an Fe domain and (b) an NKG2D-binding Fab fragment derived from A49MI including a heavy chain portion comprising a heavy chain variable domain and a CH1 domain, and a light chain portion comprising a light chain variable domain and a light chain constant domain, wherein the CH1 domain is connected to the Fc domain. F3′-GB31 includes three polypeptides: GB31-VH-VL-Fc, A49MI-VH-CH1-Fc, and A49MI-VL-CL. A49MI-VH-CH1-Fc and A49MI-VL-CL are described above in the context of F3′GB1. The polypeptide of GB31-VH-VL-Fc is set forth below.

GB31-VH-VL-Fc
(SEQ ID NO: 565)
EVQLVESGGDVVQPGRSLRLSCAASGFILSNYGMHWVRQAPGKCLEWVAAMWYDGS
NNYYEDSVKGRFTISRDNSKNTLYLQMNSLRAEDTAVYYCARERVSRHFDWHYYYGM
DVWGQGTTVTVSS
GGGGSGGGGSGGGGSGGGGS
EIVMTQSPLSLPVTPGEPASISCRSSQSLLHSNAYNYLDWYLQKPGQSPQLLISLGSIRASG
VPDRFSGSGSGTDFTLTISRVEAEDVGFYYCMQALQTPRTFGCGTKVDIT
GS
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPRVYTLPPCRDELTKNQVSLTCLVKGFYPSDIAVEWESNGQPENNYKTTPPV
LVSDGSFTLYSKLTVDKSRWQQGNVFSCSVMHEALHNHYTQKSLSLSPG

GB31-VH-VL-Fc represents the full sequence of a CEACAM5-binding scFv linked to an Fc domain via a hinge comprising Gly-Ser. The Fc domain linked to the scFv includes Q347R, D399V, and F405T substitutions for heterodimerization and an S354C substitution for forming a disulfide bond with a Y349C substitution in A49MI-VH-CH1-Fc as described above. The scFv includes a light chain variable domain of GB31 connected to the C-terminus of a heavy chain variable domain of GB31 via a (G₄S)₄ linker (SEQ ID NO: 532). The heavy and the light variable domains of the scFv are also connected through a disulfide bridge formed via cysteine heterodimerization mutations, indicated in bold-underlining in the sequence above.

In certain embodiments, a TriNKET of the present disclosure is identical to one of the exemplary TriNKETs described above that includes the EW-RVT Fc mutations, except that the Fc domain linked to the NKG2D-binding Fab fragment comprises the substitutions of Q347R, D399V, and F405T, and the Fc domain linked to the CEACAM5-binding scFv comprises matching substitutions K360E and K409W for forming a heterodimer. In certain embodiments, a TriNKET of the present disclosure is identical to one of the exemplary TriNKETs described above that includes the KiH Fc mutations, except that the Fc domain linked to the NKG2D-binding Fab fragment comprises the “hole” substitutions of T366S, L368A, and Y407V, and the Fe domain linked to the CEACAM5-binding scFv comprises the “knob” substitution of T366W for forming a heterodimer.

In certain embodiments, a TriNKET of the present disclosure is identical to one of the exemplary TriNKETs described above, except that the Fc domain linked to the NKG2D-binding Fab fragment includes an S354C substitution in the CH3 domain, and the Fc domain linked to the CEACAM5-binding scFv includes a matching Y349C substitution in the CH3 domain for forming a disulfide bond.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at its C-terminus. Accordingly, in some embodiments where the C-terminal residue of an amino acid sequence of a polypeptide (e.g., an Fc domain sequence) is K, a corresponding amino acid sequence with the K removed is also contemplated herein.

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multi-specific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

The multi-specific proteins described herein include an NKG2D-binding site, a CEACAM5-binding site, and an antibody Fc domain or a portion thereof sufficient to bind CD16, or an antigen-binding site that binds CD16. In some embodiments, the multi-specific proteins contains an additional antigen-binding site that binds to CEACAM5, as exemplified in the F4-TriNKET format.

In some embodiments, the multi-specific proteins display similar thermal stability to the corresponding monoclonal antibody, i.e., a monoclonal antibody containing the same CEACAM5-binding site as the one incorporated in the multi-specific proteins.

In some embodiments, the multi-specific proteins simultaneously bind to cells expressing NKG2D and/or CD16, such as NK cells, and cells expressing CEACAM5, such as certain tumor cells. Binding of the multi-specific proteins to NK cells can enhance the activity of the NK cells toward destruction of the CEACAM5-expressing tumor cells.

In some embodiments, the multi-specific proteins bind to CEACAM5 with a similar affinity to the corresponding anti-CEACAM5 monoclonal antibody (i.e., a monoclonal antibody containing the same CEACAM5-binding site as the one incorporated in the multi-specific proteins). In some embodiments, the multi-specific proteins are more effective in killing the tumor cells expressing CEACAM5 than the corresponding monoclonal antibodies.

In certain embodiments, the multi-specific proteins described herein, which include a binding site for CEACAM5, activate primary human NK cells when co-culturing with cells expressing CEACAM5. NK cell activation is marked by the increase in CD107a degranulation and IFN-γ cytokine production. Furthermore, compared to a corresponding anti-CEACAM5 monoclonal antibody, the multi-specific proteins can show superior activation of human NK cells in the presence of cells expressing CEACAM5.

In some embodiments, the multi-specific proteins described herein, which include a binding site for CEACAM5, enhance the activity of rested and IL-2-activated human NK cells when co-culturing with cells expressing CEACAM5.

In some embodiments, compared to the corresponding monoclonal antibody that binds to CEACAM5, the multi-specific proteins offer an advantage in targeting tumor cells that express medium and low levels of CEACAM5.

In some embodiments, the bivalent F4 format of the TriNKETs (i.e., TriNKETs including an additional antigen-binding site that binds to CEACAM5) improves the avidity with which the TriNKETs binds to CEACAM5, which in effect stabilizes expression and maintenance of high levels of CEACAM5 on the surface of the tumor cells. In some embodiments, the F4-TriNKETs mediate more potent killing of tumor cells than the corresponding F3-TriNKETs or F3′-TriNKETs.

### III. Therapeutic Applications

The invention provides methods for treating autoimmune disease or cancer using a multi-specific binding protein described herein and/or a pharmaceutical composition described herein. The methods may be used to treat a variety of cancers expressing CEACAM5.

The therapeutic method can be characterized according to the cancer to be treated. For example, in certain embodiments, the cancer is selected from the group consisting of: gastrointestinal cancer, colorectal cancer, pancreatic cancer, non-small cell lung cancer, and esophageal cancer.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

The cancer to be treated can be characterized according to the presence of a particular antigen expressed on the surface of the cancer cell. Cancers characterized by the expression of CEACAM5 include, without limitation, medullary thyroid cancer (MTC), non-medullary thyroid cancers (non-MTC), gastric cancer, colorectal cancers, hepatocellular carcinoma, lung cancer, pancreatic cancer, breast cancer, and ovarian cancer. In certain embodiments, the cancer cell can express one or more of the following: CEACAM1, CEACAM3, CEACAM6, and CEACAM8. In certain embodiments, the cancer cell can express one or more of the following in addition to CEACAM5: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

It is contemplated that the protein, conjugate, cells, and/or pharmaceutical compositions of the present disclosure can be used to treat a variety of cancers, not limited to cancers in which the cancer cells express CEACAM5. For example, in certain embodiments, the protein, conjugate, cells, and/or pharmaceutical compositions disclosed herein can be used to treat cancers that are associated with CEACAM5-expressing cells. CEACAM5 is overexpressed in a high percentage of human cancers. Therefore, the methods disclosed herein may be used to treat a variety of cancers in which CEACAM5 is expressed.

### IV. Combination Therapy

Another aspect of the invention provides for combination therapy. A multi-specific binding protein described herein can be used in combination with additional therapeutic agents to treat autoimmune disease or to treat cancer.

Exemplary therapeutic agents that may be used as part of a combination therapy in treating autoimmune inflammatory diseases are described in Li et al. (2017) Front. Pharmacol., 8:460, and include, for example, non-steroidal anti-inflammatory drugs (NSAIDs) (e.g., COX-2 inhibitors), glucocorticoids (e.g., prednisone/prednisolone, methylprednisolone, and the fluorinated glucocorticoids such as dexamethasone and betamethasone), disease-modifying antirheumatic drugs (DMARDs) (e.g., methotrexate, leflunomide, gold compounds, sulfasalazine, azathioprine, cyclophosphamide, antimalarials, D-penicillamine, and cyclosporine), anti-TNF biologics (e.g., infliximab, etanercept, adalimumab, golimumab, Certolizumab pegol, and their biosimilars), and other biologics targeting CTLA-4 (e.g., abatacept), IL-6 receptor (e.g., tocilizumab), IL-1 (e.g., anakinra), Th1 immune responses (IL-12/IL-23) (e.g., ustekinumab), Th17 immune responses (IL-17) (e.g., secukinumab) and CD20 (e.g., rituximab).

Exemplary therapeutic agents that may be used as part of a combination therapy in treating cancer include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma. In specific embodiments, the inhibitor may be an antibody, an antigen binding fragment, an immunoadhesin, a fusion protein, or oligopeptide. In some embodiments, the checkpoint inhibitor is a PD1 inhibitor selected from the group consisting of an anti-PD1 antibody or an anti-PDL1 antibody. In some embodiments, the PD1 inhibitor is chosen from nivolumab (OPDIVO, Bristol Myers Squibb, New York, New York), pembrolizumab (KEYTRUDA, Merck Sharp & Dohme Corp, Kenilworth, NJ USA), cetiplimab (Regeneron, Tarrytown, NY) or pidilizumab (CT-011). In some embodiments, the PD1 inhibitor is an immunoadhesin (e.g., an immunoadhesin comprising an extracellular or PD1 binding portion of PDL1 or PDL2 fused to a constant region (e.g., an Fc region of an immunoglobulin sequence)). In some embodiments, the PD1 inhibitor is AMP-224. In some embodiments, the PDL1 inhibitor is an antiPDL1 antibody such as durvalumab (IMFINZI, Astrazeneca, Wilmington, DE), atezolizumab (TECENTRIQ, Roche, Zurich, CH), or avelumab (BAVENCIO, EMD Serono, Billerica, MA). In some embodiments, the PDL1 inhibitor is chosen from YW243.55.S70, MPDL3280A, MEDI-4736, MSB-0010718C, or MDX-1105.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from the group consisting of: an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from the group consisting of: IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized). In certain embodiments, the formulation may be a liquid formulation.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996).

In embodiments, the protein product of the present disclosure is formulated as a liquid formulation. The liquid formulation may be presented in either a USP/Ph Eur type I 50R vial closed with a rubber stopper and sealed with an aluminum crimp seal closure. The stopper may be made of elastomer complying with USP and Ph Eur. In certain embodiments vials may be filled with the protein product solution in order to allow an extractable volume. In certain embodiments, the liquid formulation may be diluted with saline solution.

In certain embodiments, the liquid formulation of the disclosure may be prepared in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The protein of the present disclosure could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is reconstituted with water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1. Immunization and Generation of Hybridomas

BALB/cJ mice were purchased from The Jackson Laboratory (Stock No:000651) and immunized with either: isogenic Ba/F3 cell lines over-expressing cyno and/or human CEACAM5; hCEACAM5-A3B3-domain; recombinant cyno and/or human CEACAM5; or human NABA construct (NABA consisting of the N, A1, and A2 domains of human CEACAM1 and the B3 domain of human CEACAM5). These mice were used for the production of murine mAbs (monoclonal antibodies). H2L2™ mice were purchased from Harbour Biomed and immunized with either: isogenic Ba/F3 cell lines over-expressing cyno and/or human CEACAM5; hCEACAM5-A3B3-domain; recombinant cyno and/or human CEACAM5; or human NABA construct (NABA consisting of the N, A1, and A2 domains of human CEACAM1 and the B3 domain of human CEACAM5). These mice were used for the production of human/rat chimeric mAbs with human variable regions and rat constant regions. Thereafter, the spleen cells from immunized mice were fused with mouse myeloma cells to generate hybridoma cells.

The fused hybridoma cells were cultured in supplemented DMEM culture media in humidified air at 37° C. with 8% CO₂. Supernatants of the hybridomas were assessed for CEACAM5, CEACAM1, CEACAM6 and CEACAM8 binding by enzyme-linked immunosorbent assay (ELISA) and multiplex surface plasmon resonance (SPR) (Carterra LSA). CEACAM5 antigen-specific hybridomas were subsequently subcloned. Clones for further study were selected based on preliminary multiplex SPR (Carterra) binding affinity estimations, binding to cells expressing human and cynomolgus monkey CEACAM5, binding to CEACAM5⁺ cancer cell lines, and diversity of epitopes. Cross-reactivity with cynomolgus monkey CEACAM5 was observed for Clone 16F6.A2-CEACAM5-B.02.

The Balb/cJ murine mAbs were purified from hybridoma supernatants by Protein A chromatography using AmMag™ Protein A magnetic beads (P/N L00695, Genscript Biotech, Piscataway, NJ). The beads were equilibrated with 1.5M Glycine, 3.0M NaCl pH 8.5. The supernatants were diluted 1:1 with 1.5M Glycine, 3.0M NaCl pH 8.5 and incubated with ProA Magnetic beads with gentle rocking for 1 h. Beads were washed with 1.5M Glycine, 3.0M NaCl pH 8.5 to remove unbound proteins. The antibodies were eluted with 100 mM Glycine pH 3.0 and immediately neutralized to pH 7.5 with 1.0M Tris, pH 8.3. Protein concentration was determined by A280 using a Nanodrop spectrophotometer. The H2L2 derived human (variable region)/rat (constant region) mAbs were purified from hybridoma supernatants by Protein G chromatography (Global Life Sciences Solutions, Marlborough, MA). The Protein G column was equilibrated with 50 mM Sodium Acetate, pH 5.0+10 mM NaCl. The supernatants were diluted 10-fold with 50 mM Sodium Acetate, pH 5.0+10 mM NaCl and with equilibrated Protein G media with gentle rocking for 1 h. Beads were washed with 50 mM Sodium Acetate, pH 5.0+10 mM NaCl to remove unbound proteins. The antibodies were eluted with 100 mM Glycine pH 2.5 with immediate neutralization to pH 7.5 with LOM Tris, pH 8.3. Following purification, protein concentration was determined by A280 using a Nanodrop spectrophotometer. Similarly, the H2L2 derived human/rat chimeric mAbs (having human variable regions and rat constant regions) were purified from hybridoma supernatants by Protein G chromatography (Global Life Sciences Solutions, Marlborough, MA); protein concentration was determined by A280 using a Nanodrop spectrophotometer.

The Balb/cJ murine mAbs were tested for cell surface binding to CEACAM5 and cross-reactivity with CEACAM1, CEACAM6, and CEACAM8. Additionally, the Balb/cJ murine mAbs were tested for in vitro binding to CEACAM1, CEACAM5, CEACAM6, and CEACAM8 using surface plasmon resonance (SPR). The experiment was performed at 37° C. to mimic physiological temperature using either Carterra LSA or a Biacore 8K instrument.

The H2L2-derived human/rat chimeric mAbs were tested for cell surface binding to CEACAM5 and cross-reactivity with CEACAM1, CEACAM6, and CEACAM8. Additionally, the H2L2-derived human/rat chimeric mAbs were tested for in vitro binding to CEACAM5, CEACAM1, CEACAM6, and CEACAM8 using surface plasmon resonance (SPR). The experiment was performed at 37° C. to mimic physiological temperature using either Carterra LSA or a Biacore 8K instrument.

### Example 2. Generation of Binders with Yeast Display Technology

Additional CEACAM5 binders were generated using yeast display technology by building H2L2 immune libraries. Briefly, yeast were transfected with starter constructs comprising parental CEACAM5 VH and/or VL sequences. Novel clones were selected for, and binders were isolated and characterized as described supra.

### Yeast Strains and Plasmids

Auxotrophic MATa Saccharomyces cerevisiae strain EBY100 (Meyen ex E.C. Hansen (ATCC® MYA-4941™)), with leucine and tryptophan selectable markers, was used for construction of the scFv yeast display library. EBY100 has a genomic insertion of AGA1 for surface-display; its expression is regulated by the galactose promoter with a uracil selectable marker. Each scFv also contains a carboxy-terminal flag tag also controlled by the galactose promoter.

Construction of CEACAM5 Immune scFv Yeast Display Library

After the immunization procedure, mouse splenocytes were collected, RNA was extracted with a high pure RNA isolation kit (Roche, product no. 11828665001), and RT-PCR was performed to generate cDNA libraries (Invitrogen, 18090010) according to the manufacturer's instructions. The cDNA was used as a template for the amplification of variable heavy-chain (VH) and light-chain (VL; kappa only) antibody genes, which were then assembled into single-chain antibody fragment (scFv) libraries in both orientations (VH-VL or VL-VH scFvs). The scFv libraries were integrated into a yeast surface display vector after transformation (electroporation) and homologous recombination.

Isolation of CEACAM5 Specific scFv Binders from the Library

After electroporation, yeast cells were grown in the selectable media (Teknova product no. C8240). The scFv libraries were then induced to display scFvs on the yeast cell surface by switching to the galactose media. Isolation of CEACAM5 specific binders was accomplished by 3 rounds of selection. The library was first screened with human CEACAM5-hits using magnetic activated cell sorting (MACS) followed by two rounds of selection by fluorescence-activated cell sorting (FACS), resulting in a panel of CEACAM5 specific scFvs. Individual scFv clones were characterized for CEACAM5 binding affinity and specificity while displayed on yeast, and then moved into vectors for expression in mammalian cells using molecular biology techniques known in the art.

### Example 3. Humanization and Creation of Variants for Sequence Liabilities

The Balb/cJ Hybridoma mAbs were humanized. Humanized variants were generated by grafting murine CDRs onto human framework regions. The following clones were humanized: murine 1A1.A3-CEACAM5-B.02 and murine 16F6.A2-CEACAM5-B.02. Their sequences and those of their humanized variants are provided in Table 4. The murine VH and VL sequences of clone 16F6.A2-CEACAM5-B.02 were blast against the human sequence database to identify the appropriate framework for hosting the CDRs. IGHV1-2*02 (sequence identity: 66.3%) and IGKV4-1*01 (sequence identity: 80.2%) were selected to move forward. A structural model was built to inspect and identify potential back mutations. After grafting the hypervariable regions from the murine corresponding antibody to the above human frameworks, the following back mutations were introduced in the variable heavy chain (VH): V67T, M69L, R71A, S76P and A93T (all under Chothia numbering). There were no back mutations in the variable light chain (VL).

The murine VH and VL sequences of clone 1A1.A3 were blast against the human sequence database to identify the appropriate framework for hosting the CDRs. IGHV1-69-2*01 (sequence identity: 63.5%) and IGKV3-11*01 (sequence identity: 64.2%) were selected to move forward. A structural model was built to inspect and identify potential back mutations. After grafting the hypervariable regions from the murine corresponding antibody to the above human frameworks, the following back mutations in VH were introduced: V5Q, K12V, I20L, V24A, Q38T, M48I, V67A, I69M, M80L, A93N and T94V (all under Chothia numbering). The following back mutation in the VL were also introduced: L13A, Y36F, L47W, I58V and F71Y.

Potential sequence liabilities of the clones were examined. The following potential sequence liabilities were considered: M (potential oxidation site); NG, NS and NT sequence motif (potential deamidation site); DG, DS and DT sequence motif (potential isomerization site); and DP sequence motif (potential site for chemical hydrolysis). Variants of these antibodies were designed to remove the putative sequence liability motifs; the sequences of such variants are provided in Table 4.

### Example 4. Classification of Monoclonal Antibodies

The mAbs were classified by Tier (Tiers 1, 2, and 3), as indicated in Tables 3 and 4. mAbs classified as “Tier 1” were CEACAM5-specific high affinity mAbs, with KD values less than or equal to about 15 nM (e.g., a KD range of about 4 nM to about 15 nM). mAbs classified as Tier 2 were CEACAM5-specific medium and low affinity mAbs, with KD values ranging from about 25 nM to about nM to about 80 nM (for medium affinity), and about 120 nM to about 560 nM (for low affinity). mAbs classified as “Tier 3” were mAbs that, in addition to binding to CEACAM5, displayed a low level of cross-reactivity with CEACAM1, CEACAM6, and CEACAM8. For the purpose of the classification, the mAbs generated by the method of Example 1 were assessed in the mAb format, and the mAbs generated by the method of Example 2 were assessed in the format of a multi-specific antibody containing an scFv that binds CEACAM5 and an Fab fragment that binds a different antigen.

The clones of each tier are listed in Table 13 below. The VH, VL, and CDR sequences of these clones are provided in Table 4.

TABLE 13
Classification of Antibody Clones

| Tier | Source Library | Clones |
|---|---|---|
| Tier 1 | H2L2 immune yeast | Clone PH_420-CEACAM5 |
| | library (fully human | 1078_C04-CEACAM5 |
| | antibodies) | 1079_H05-CEACAM5 |
| Tier 1 | H2L2 hybridoma | 7A10.A7-CEACAM5-B.01 |
| | library (fully human | 8H2.B10-CEACAM5-B.01 |
| | antibodies) | |
| Tier 1 | Balb/cJ hybridoma | Murine 16F6.A2-CEACAM5-B.02 |
| | library (murine and | Humanized 16F6.A2-CEACAM5-B.02-BM |
| | humanized | |
| | antibodies) | |
| Tier 2 | H2L2 immune yeast | PH_415-CEACAM5 |
| | library (fully human | PH_416-CEACAM5 |
| | antibodies) | PH_417-CEACAM5 |
| | | PH_418-CEACAM5 |
| | | PH_419-CEACAM5 |
| | | PH_421-CEACAM5 |
| | | 1078_G03-CEACAM5 |
| | | 1081_E01-CEACAM5 |
| Tier 2 | Balb/cJ hybridoma | Murine 1A1.A3-CEACAM5-B.02 |
| | library (murine and | Humanized 1A1.A3-CEACAM5-B.02-BM |
| | humanized | 7E11.B2-CEACAM5-B.02 |
| | antibodies) | 4B10.B3-CEACAM5-A.02 |
| | | 10D6.E3-CEACAM5-B.02 |
| | | 13C7.A6-CEACAM5-B.02 |
| | | 13C7.F2-CEACAM5-B.02 |
| | | 16B11.G2-CEACAM5-B.01 |
| Tier 2 | H2L2 hybridoma | 12A6.H2-CEACAM5-B.01 |
| | library (fully human | 4G3.C3-CEACAM5-B.01 |
| | antibodies) | |
| Tier 3 | H2L2 immune yeast | 1080_G01-CEACAM5 |
| | library (fully human | 1078_C12-CEACAM5 |
| | antibodies) | 1078_F02-CEACAM5 |
| | | 1078_G03-CEACAM5 |
| | | 1079_A10-CEACAM5 |
| | | 1079_A12-CEACAM5 |
| | | 1079_B08-CEACAM5 |
| | | 1078_C04-CEACAM5 |
| | | 1080_F11-CEACAM5 |
| | | 1083_A05-CEACAM5 |
| | | 1085_D12-CEACAM5 |
| | | 1079_G12-CEACAM5 |
| | | 1080_A01-CEACAM5 |
| Tier 3 | H2L2 hybridoma | 12C7.A2-CEACAM5-B.01 |
| | library (fully human | |
| | antibodies) | |
| Tier 3 | Balb/cJ hybridoma | |
| | library (murine and | |
| | humanized | 6D10.C8-CEACAM5-B.02 |
| | antibodies) | |

### Example 5. NKG2D Binding Domains Bind to NKG2D

### Binding Affinities of Various NKG2M-Binding Domains

Kinetics and affinity of various NKG2D-binding domains were assessed by surface plasmon resonance using Biacore 8K instrument (GE Healthcare). Anti-human Fe antibody was immobilized on a CM5 chip using standard amine coupling chemistry. Human monoclonal antibodies containing various NKG2D-binding domains were captured on the anti-human Fc chip at a density of approximately 100 RU. Solutions containing 0.411-100 nM soluble mouse Fc-human NKG2D dimers were injected over the captured NKG2D antibodies and control surfaces at 30 μl/min at 37° C. Surfaces were regenerated between cycles by quick injection of 10 mM glycine, pH 1.8. To obtain kinetic rate constants, double-referenced data were fit to a 1:1 interaction model using Biacore 8K Evaluation software (GE Healthcare). The equilibrium binding constant K_{D} was determined by the ratio of dissociation constant k_{d} and association constant kₐ (k_{d}/kₐ). As shown in Table 14 below, binding affinities of NKG2D-binding domains to NKG2D are in the range of 10-62 nM.

TABLE 14
Binding Affinity of NKG2D Binding Domains

| NKG2D-binding domain | kₐ (1/Ms) | kₐ (1/s) | K_{D} (nM) |
|---|---|---|---|
| ADI-27744 (A44) | 2.95E+05 | 2.99E−03 | 10.1 |
| ADI-27749 (A49) | 3.95E+05 | 4.89E−03 | 12.4 |
| ADI-29378 (E78) | 8.32E+05 | 4.87E−02 | 58.5 |
| ADI-29379 (E79) | 4.43E+05 | 2.25E−02 | 50.7 |
| ADI-29463 (F63) | 1.64E+06 | 1.01E−01 | 61.8 |

### NKG2D-Binding Domains Bind to Purified Recombinant NKG2D

The nucleic acid sequences of human, mouse or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D-binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D-binding domain clone, an isotype control or a positive control (comprising heavy chain and light chain variable domains selected from the group consisting of: SEQ ID NOs: 526-529, or anti-mouse NKG2D clones MI-6 and CX-5 available at eBioscience) was added to each well.

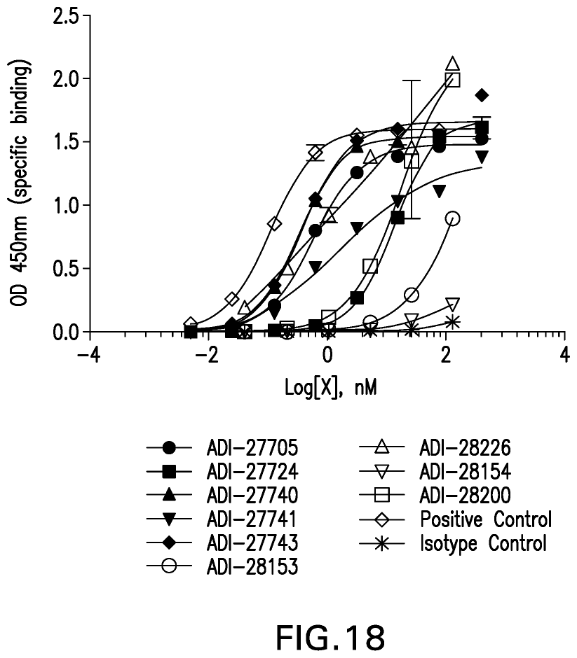

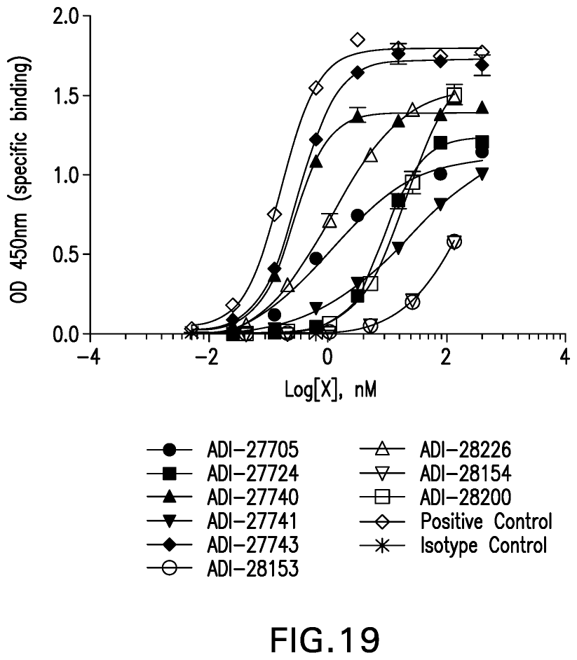

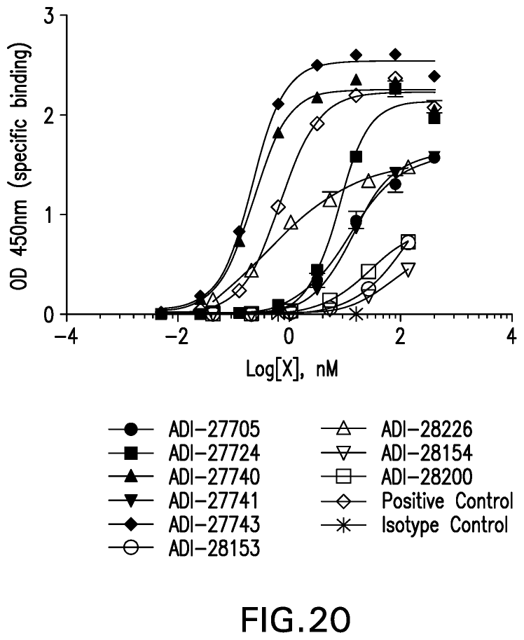

The isotype control showed minimal binding to recombinant NKG2D-Fc proteins, while the positive control bound strongest to the recombinant antigens. NKG2D-binding domains produced by all clones demonstrated binding across human, mouse, and cynomolgus recombinant NKG2D-Fc proteins, although with varying affinities from clone to clone. Generally, each anti-NKG2D clone bound to human (FIG. 18) and cynomolgus (FIG. 19) recombinant NKG2D-Fc with similar affinity, but with lower affinity to mouse (FIG. 20) recombinant NKG2D-Fc.

### NKG2D-Binding Domains Bind to Cells Expressing NKG2D

EL4 mouse lymphoma cell lines were engineered to express human or mouse NKG2D-CD3 zeta signaling domain chimeric antigen receptors. An NKG2D-binding clone, an isotype control or a positive control was used at a 100 nM concentration to stain extracellular NKG2D expressed on the EL4 cells. The antibody binding was detected using fluorophore-conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

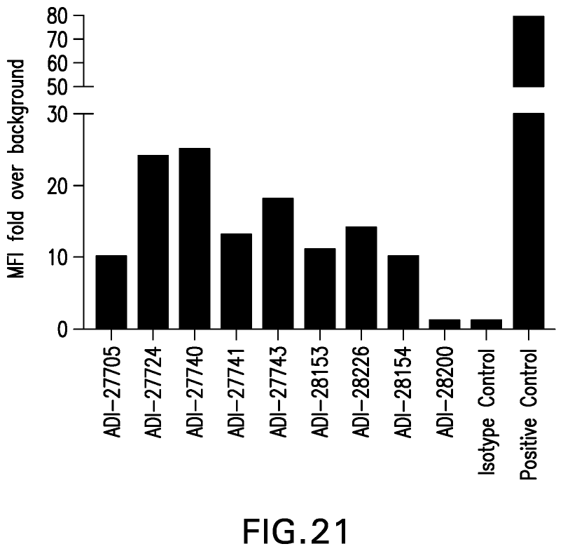

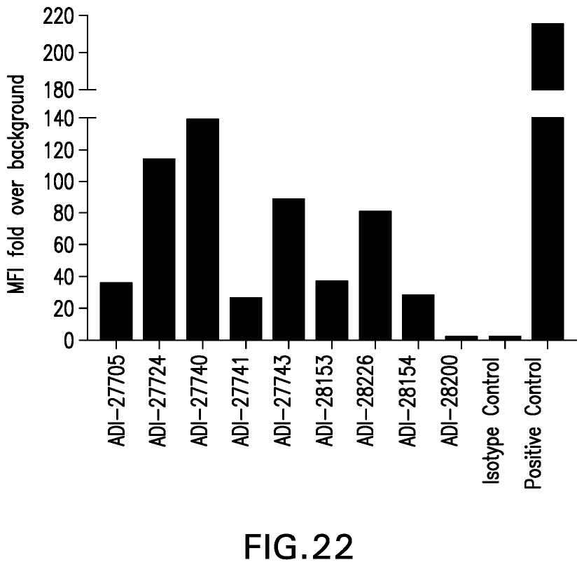

NKG2D-binding domains produced by all clones bound to EL4 cells expressing human and mouse NKG2D. Positive control antibodies (comprising heavy chain and light chain variable domains selected from the group consisting of: SEQ ID NOs: 526-529, or anti-mouse NKG2D clones MI-6 and CX-5 available at eBioscience) gave the best FOB binding signal. The NKG2D-binding affinity for each clone was similar between cells expressing human NKG2D (FIG. 21) and mouse (FIG. 22) NKG2D.

### Example 6. NKG2D-Binding Domains Block Natural Ligand Binding to NKG2D

Competition with ULBP-6

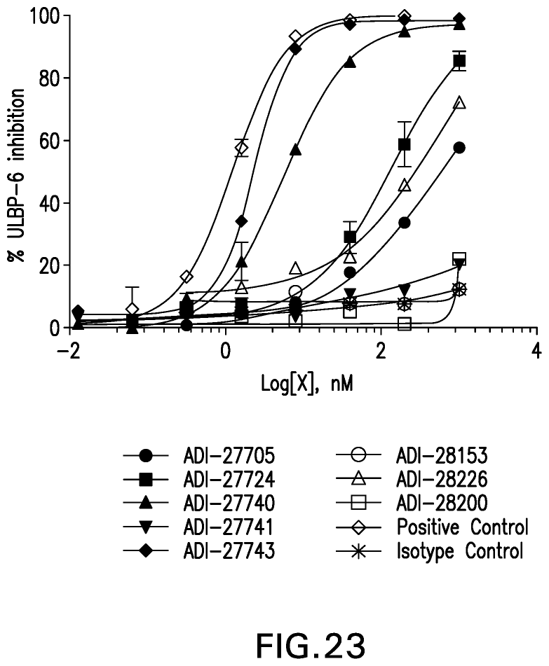

Recombinant human NKG2D-Fc proteins were adsorbed to wells of a microplate, and the wells were blocked with bovine serum albumin to reduce non-specific binding. A saturating concentration of ULBP-6-His-biotin was added to the wells, followed by addition of the NKG2D-binding domain clones. After a 2-hour incubation, wells were washed and ULBP-6-His-biotin that remained bound to the NKG2D-Fc coated wells was detected by streptavidin-conjugated to horseradish peroxidase and TMB substrate. Absorbance was measured at 450 nM and corrected at 540 nM. After subtracting background, specific binding of NKG2D-binding domains to the NKG2D-Fc proteins was calculated from the percentage of ULBP-6-His-biotin that was blocked from binding to the NKG2D-Fc proteins in wells. The positive control antibody (comprising heavy chain and light chain variable domains selected from the group consisting of: SEQ ID NOs: 526-529) and various NKG2D-binding domains blocked ULBP-6 binding to NKG2D, while isotype control showed little competition with ULBP-6 (FIG. 23).

ULBP-6 sequence is represented by SEQ ID NO:566.

(SEQ ID NO: 566)
MAAAAIPALLLCLPLLFLLFGWSRARRDDPHSLCYDITVIPKFRPGPRW
CAVQGQVDEKTFLHYDCGNKTVTPVSPLGKKLNVTMAWKAQNPVLREVV
DILTEQLLDIQLENYTPKEPLTLQARMSCEQKAEGHSSGSWQFSIDGQT
FLLFDSEKRMWTTVHPGARKMKEKWENDKDVAMSFHYISMGDCIGWLED
FLMGMDSTLEPSAGAPLAMSSGTTQLRATATTLILCCLLIILPCFILPG
I

Competition with MICA

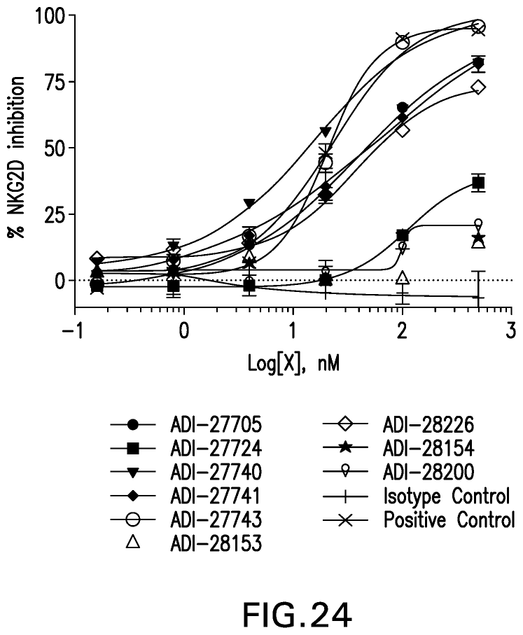

Recombinant human MICA-Fc proteins were adsorbed to wells of a microplate, and the wells were blocked with bovine serum albumin to reduce non-specific binding. NKG2D-Fc-biotin was added to wells followed by NKG2D-binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the MICA-Fc coated wells. The positive control antibody (comprising heavy chain and light chain variable domains selected from the group consisting of: SEQ ID NOs: 526-529) and various NKG2D-binding domains blocked MICA binding to NKG2D, while isotype control showed little competition with MICA (FIG. 24).

Competition with Rae-1 Delta

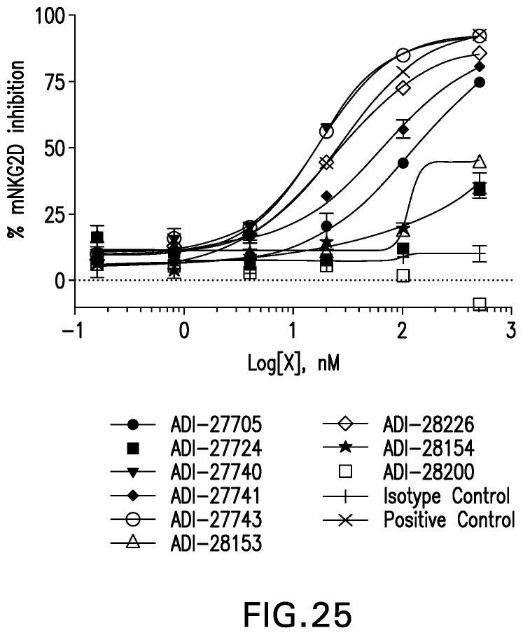

Recombinant mouse Rae-1delta-Fc (purchased from R&D Systems) was adsorbed to wells of a microplate, and the wells were blocked with bovine serum albumin to reduce non-specific binding. Mouse NKG2D-Fc-biotin was added to the wells followed by NKG2D-binding domains. After incubation and washing, NKG2D-Fc-biotin that remained bound to Rae-1delta-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the Rae-1delta-Fc coated wells. The positive control (comprising heavy chain and light chain variable domains selected from the group consisting of: SEQ ID NOs: 526-529, or anti-mouse NKG2D clones MI-6 and CX-5 available at eBioscience) and various NKG2D-binding domain clones blocked Rae-1delta binding to mouse NKG2D, while the isotype control antibody showed little competition with Rae-1delta (FIG. 25).

### Example 7. NKG2D-Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

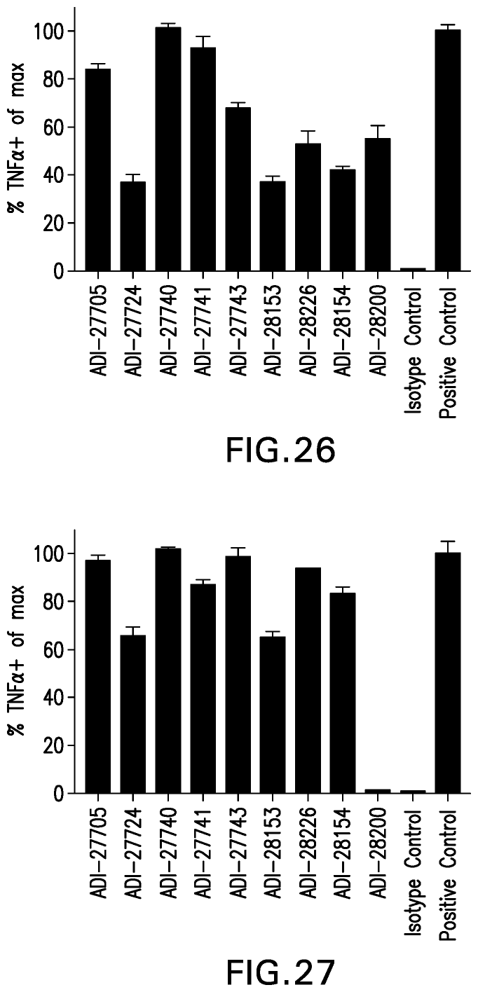

To determine whether NKG2D-binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-α production, an indicator for NKG2D activation, was assayed by flow cytometry. The percentage of TNF-α positive cells was normalized to the cells treated with the positive control. All NKG2D-binding domains activated both human NKG2D (FIG. 26) and mouse NKG2D (FIG. 27).

### Example 8. NKG2D-Binding Domains Activate NK Cells

### Primary Human NK Cells

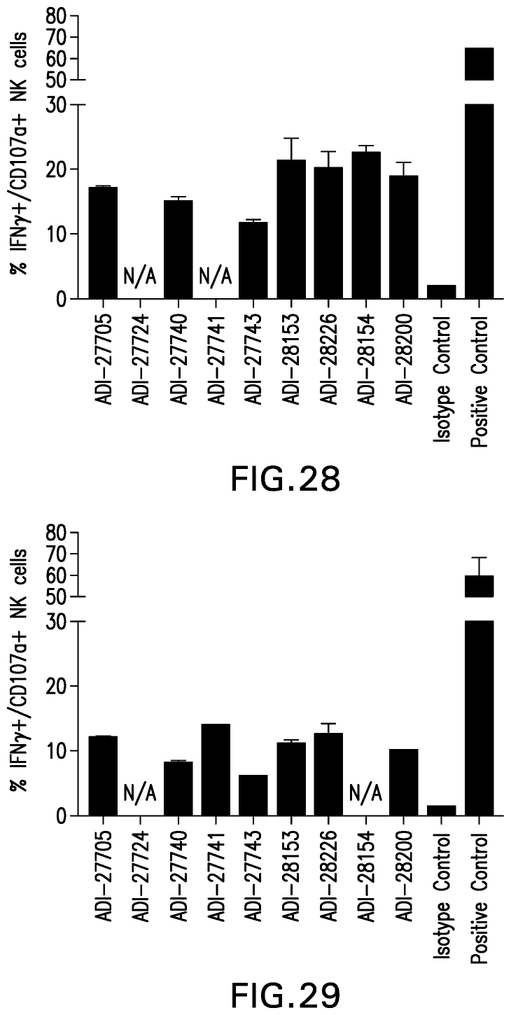

Peripheral blood mononuclear cells (PBMCs) were isolated from human peripheral blood buffy coats using density gradient centrifugation. NK cells (CD3⁻ CD56*) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D-binding domains were adsorbed, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFN-γ. CD107a and IFN-γ staining were analyzed in CD3⁻ CD56⁺ cells to assess NK cell activation. The increase in CD107a/IFN-γ double-positive cells is indicative of better NK cell activation through engagement of two activating receptors rather than one receptor. NKG2D-binding domains and the positive control (e.g., heavy chain variable domain represent by SEQ ID NO:526 or SEQ ID NO:528, and light chain variable domain represented by SEQ ID NO:527 or SEQ ID NO:529) showed a higher percentage of NK cells becoming CD107a⁺ and IFN-γ⁺ than the isotype control (FIG. 28 and FIG. 29 represent data from two independent experiments, each using a different donor's PBMC for NK cell preparation).

### Primary Mouse NK Cells

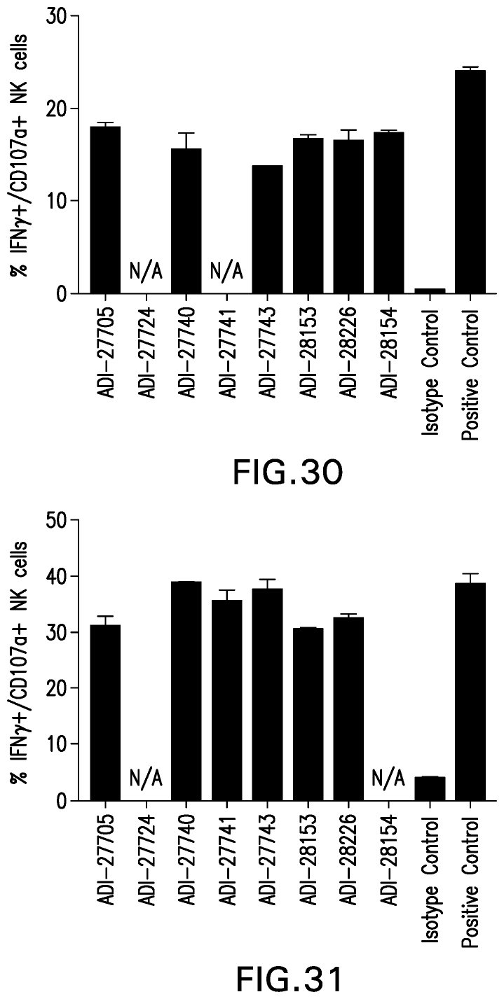

Spleens were obtained from C57Bl/6 mice and crushed through a 70 μm cell strainer to obtain single cell suspension. Cells were pelleted and resuspended in ACK lysis buffer (purchased from Thermo Fisher Scientific #A1049201; 155 mM ammonium chloride, 10 mM potassium bicarbonate, 0.01 mM EDTA) to remove red blood cells. The remaining cells were cultured with 100 ng/mL hIL-2 for 72 hours before being harvested and prepared for NK cell isolation. NK cells (CD3⁻NK1.1⁺) were then isolated from spleen cells using a negative depletion technique with magnetic beads with typically >90% purity. Purified NK cells were cultured in media containing 100 ng/mL mIL-15 for 48 hours before they were transferred to the wells of a microplate to which the NKG2D-binding domains were adsorbed, and cultured in the media containing fluorophore-conjugated anti-CD107a antibody, brefeldin-A, and monensin. Following culture in NKG2D-binding domain-coated wells, NK cells were assayed by flow cytometry using fluorophore-conjugated antibodies against CD3, NK1.1 and IFN-γ. CD107a and IFN-γ staining were analyzed in CD3-NK1.1⁺ cells to assess NK cell activation. The increase in CD107a/IFN-γ double-positive cells is indicative of better NK cell activation through engagement of two activating receptors rather than one receptor. NKG2D-binding domains and the positive control (selected from the group consisting of: anti-mouse NKG2D clones MI-6 and CX-5 available at eBioscience) showed a higher percentage of NK cells becoming CD107a⁺ and IFN-γ⁺ than the isotype control (FIG. 30 and FIG. 31 represent data from two independent experiments, each using a different mouse for NK cell preparation).

### Example 9. NKG2D-Binding Domains Enable Cytotoxicity of Target Tumor Cells

Human and mouse primary NK cell activation assays demonstrated increased cytotoxicity markers on NK cells after incubation with NKG2D-binding domains. To address whether this translates into increased tumor cell lysis, a cell-based assay was utilized where each NKG2D-binding domain was developed into a monospecific antibody. The Fc region was used as one targeting arm, while the Fab fragment region (NKG2D-binding domain) acted as another targeting arm to activate NK cells. THP-1 cells, which are of human origin and express high levels of Fc receptors, were used as a tumor target and a Perkin Elmer DELFIA Cytotoxicity Kit was used. THP-1 cells were labeled with BATDA reagent, and resuspended at 10⁵/mL in culture media. Labeled THP-1 cells were then combined with NKG2D antibodies and isolated mouse NK cells in wells of a microtiter plate at 37° C. for 3 hours. After incubation, 20 μL of the culture supernatant were removed, mixed with 200 μL of Europium solution and incubated with shaking for 15 minutes in the dark. Fluorescence was measured over time by a PheraStar plate reader equipped with a time-resolved fluorescence module (Excitation 337 nm, Emission 620 nm) and specific lysis was calculated according to the kit instructions.

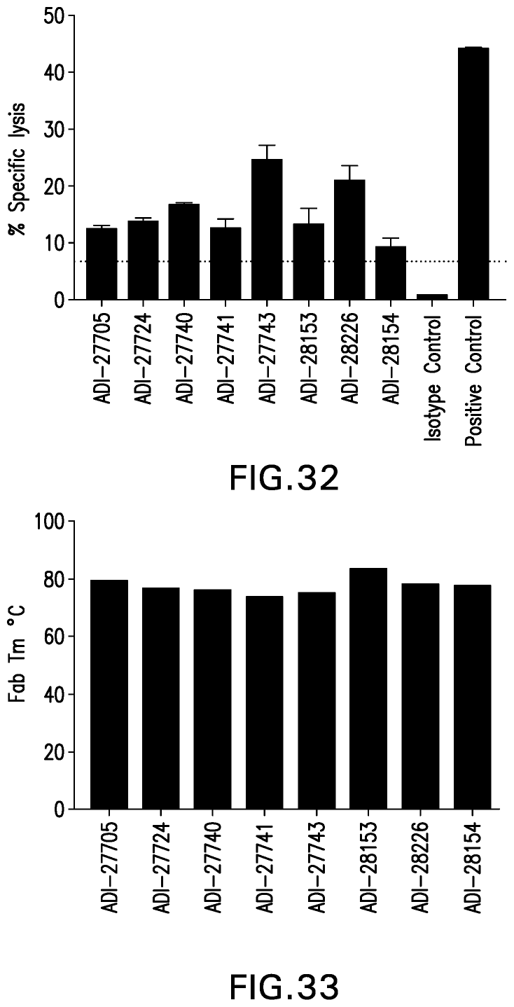

The positive control, ULBP-6—a natural ligand for NKG2D, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 32).

### Example 10. NKG2D Antibodies Show High Thermostability

Melting temperatures of NKG2D-binding domains were assayed using differential scanning fluorimetry. The extrapolated apparent melting temperatures are high relative to typical IgG1 antibodies (FIG. 33).

### Example 11. Synergistic Activation of Human NK Cells by Cross-Linking NKG2D and CD16

### Primary Human NK Cell Activation Assay

Peripheral blood mononuclear cells (PBMCs) were isolated from peripheral human blood buffy coats using density gradient centrifugation. NK cells were purified from PBMCs using negative magnetic beads (StemCell, #17955). NK cells were >90% CD3-CD56⁺ as determined by flow cytometry. Cells were then expanded 48 hours in media containing 100 ng/mL hIL-2 (Peprotech, #200-02) before use in activation assays. Antibodies were coated onto a 96-well flat-bottom plate at a concentration of 2 μg/mL (anti-CD16, Biolegend, #302013) and 5 μg/mL (anti-NKG2D, R&D #MAB139) in 100 μL sterile PBS overnight at 4° C. followed by washing the wells thoroughly to remove excess antibody. For the assessment of degranulation IL-2-activated NK cells were resuspended at 5×10⁵ cells/mL in culture media supplemented with 100 ng/mL human IL-2 (hIL2) and 1 μg/mL APC-conjugated anti-CD107a mAb (Biolegend #328619). 1×10⁵ cells/well were then added onto antibody coated plates. The protein transport inhibitors Brefeldin A (BFA, Biolegend #420601) and Monensin (Biolegend #420701) were added at a final dilution of 1:1000 and 1:270, respectively. Plated cells were incubated for 4 hours at 37° C. in 5% CO₂. For intracellular staining of IFN-γ, NK cells were labeled with anti-CD3 (Biolegend #300452) and anti-CD56 mAb (Biolegend #318328), and subsequently fixed, permeabilized and labeled with anti-IFN-γ mAb (Biolegend #506507). NK cells were analyzed for expression of CD107a and IFN-γ by flow cytometry after gating on live CD56+CD3-cells.

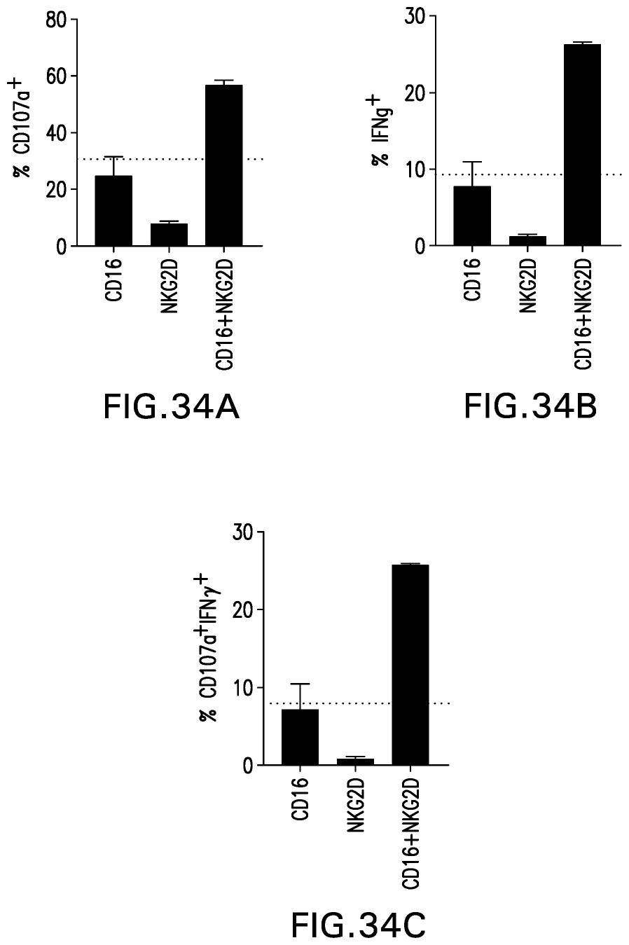

To investigate the relative potency of receptor combination, crosslinking of NKG2D or CD16, and co-crosslinking of both receptors by plate-bound stimulation was performed. As shown in FIG. 34 (FIGS. 34A-34C), combined stimulation of CD16 and NKG2D resulted in highly elevated levels of CD107a (degranulation) (FIG. 34A) and/or IFN-γ production (FIG. 34B). Dotted lines represent an additive effect of individual stimulations of each receptor.

CD107a levels and intracellular IFN-γ production of IL-2-activated NK cells were analyzed after 4 hours of plate-bound stimulation with anti-CD16, anti-NKG2D or a combination of both monoclonal antibodies. Graphs indicate the mean (n=2)±SD. FIG. 34A demonstrates levels of CD107a; FIG. 34B demonstrates levels of IFN-γ; FIG. 34C demonstrates levels of CD107a and IFN-γ. Data shown in FIGS. 34A-34C are representative of five independent experiments using five different healthy donors.

### Example 12. Generation of CEACAM5 TriNKET Binding Proteins

Creation of AB0264 and AB0621 scFvs from AB0131

AB0131 is a scFv identified from the BALB/c immunization efforts described in Example 1. AB0131 was derived from clone 16F6.A2-CEACAM5-B.02-BM. To generate humanized AB0264, five (5) backmutations were introduced into the VH domain of AB0131, and two (2) cysteines were introduced to stabilize disulfide bonds. The scFv polypeptide sequence of AB0264 is set forth below as SEQ ID NO:703. Backmutations are identified in bold lettering and introduced cysteines are identified in bold underlining.

(SEQ ID NO: 703)
DIVMTQSPDSLAVSLGERATINCKSSHSLLYGNFQNNYLAWYQQKPGQP
PKLLIYWASTRESGVPDRFSGSGSGTDFTLTISSLQAEDVAVYYCQQYY
SYPYTFGCGTKLEIKGGGGSGGGGSGGGGSGGGGSQVQLVQSGAEVKKP
GASVKVSCKASGYTFTAYTIHWVRQAPGQCLEWMGYINPSSGYTEYNQK
FKDRTTLTADTSIPTAYMELSRLRSDDTAVYYCTREGGLLWFDYWGQGT
LVTVSS)

A proline residue in the VH domain of AB0264 was found to be present at <1% frequency in human frameworks. This residue, identified in bold italics, was substituted with seine (36% of human framework sequences; Abysis) to generate AB0621. The scFv polypeptide sequence of AB0621 is set forth below as SEQ ID NO:714.

(SEQ ID NO: 714)
DIVMTQSPDSLAVSLGERATINCKSSHSLLYGNFQNNYLAWYQQKPGQP
PKLLIYWASTRESGVPDRFSGSGSGTDFTLTISSLQAEDVAVYYCQQYY
SYPYTFGCGTKLEIKGGGGSGGGGSGGGGSGGGGSQVQLVQSGAEVKKP
GASVKVSCKASGYTFTAYTIHWVRQAPGQCLEWMGYINPSSGYTEYNQK
FKDRTTLTADTSISTAYMELSRLRSDDTAVYYCTREGGLLWFDYWGQGT
LVTVSS

Creation of AB0411 scFv from AB0100 scFv

AB0100 is a fully human scFv identified from interrogation of the H2L2 yeast immune library described in Example 2. AB0100 was derived from a variant of clone 1078_C04CEACAM5. A glutamine residue in the VH domain of AB0100 was found to be present in <1% of human frameworks. To generate AB0411, the glutamine residue was substituted with a leucine (63% of human frameworks; Abysis), and two (2) cysteines were introduced to stabilize disulfide bonds. The scFv polypeptide sequence of AB0411 is set forth below as SEQ ID NO:707. Stabilizing cysteines are identified in bold underlining and the leucine substitution is shown in bold italics.

(SEQ ID NO: 707)
DIQLTQSPATLSVSPGERATLSCRASQSVSSSYLAWYQQKPGQAPRLLI
YGASSRATGIPDRFSGSGSGTDFTLTISRLEPEDFAVYYCQQYNNWPLT
FGCGTKVEIKGGGGSGGGGSGGGGSGGGGSQVQLVQSGGGLVQPGGSLR
LSCAASGFTFTSYAMSWVRQAPGKCLEWVSAISGTGDSTFYADSVKGRF
TFSRDNSKNTLYLQMNSLRAEDTAVYYCAKDLGWLQYGLFDYWGQGTLV
TVSS

Creation of AB0466 scFv from AB0073 scFv

AB0073 is a fully human scFv identified from interrogation of the H2L2 yeast immune library described in Example 2. AB0073 was derived from a variant of clone PH_420-CEACAM5. An arginine residue in the VL domain of AB0073 was found to be present in less than <1% of human frameworks. To generate AB0466, the arginine residue was substituted with a glutamine (15% of human frameworks; Abysis). In addition, an NS motif in the VH domain was substituted with an SS and an NS motif of the VL domain was substituted with an NA because the NS motifs were confirmed to undergo deamidation following stress. Finally, two (2) cysteines were introduced to stabilize disulfide bonds. The scFv polypeptide sequence of AB0466 is set forth below as SEQ ID NO:710. Stabilizing cysteines are identified in bold underlining and all other amino acid substitutions are shown in bold italics.

(SEQ ID NO: 710)
DIQMTQSPSTLSASVGDRVTITCWASQSISSWLAWYQQKPGKAPKLLIY
KASSLESGVPSRFSGSGSGTEFTLTISSLQPDDFATYYCQQYNAYSYTF
GCGTKLEIKGGGGSGGGGSGGGGSGGGGSQVQLVQSGGGLVQPGGSLRL
SCAASGFTFSSYAMSWVRQAPGKCLEWVSAIFSSGGSTYYADSVKGRFT
VSRDNSKNTLYLQMNSLRAEDTALYYCAKDLGGYNYGLFDYWGQGTLVT
VSS

### Creation of F3′ CEACAM5 TriNKET Multispecific Binding Proteins

The AB0264, AB0621, AB0411, and AB0466 scFvs described above were redesigned as F3′ TriNKET multispecific binding proteins. The F3′ TriNKETs comprised:
(a) a CEACAM5-binding scFv sequence including a light chain variable domain connected to the C-terminus of a heavy chain variable domain via a (G₄S)₄ linker (SEQ ID NO: 532), wherein the scFv is linked to an Fc domain and the Fc domain includes Q347R, D399V, and F405T substitutions for heterodimerization and an S354C substitution for disulfide bond formation; and
(b) an NKG2D-binding Fab fragment derived from A49M, including a heavy chain portion comprising a heavy chain variable domain and a CH1 domain, and a light chain portion comprising a light chain variable domain and a light chain constant domain, wherein the CH1 domain is linked to an Fc domain, and wherein the Fc domain includes K360E and K409W substitutions for heterodimerization and a Y349C substitution for disulfide bond formation. The amino acid sequences of the F3′ CEACAM5 TriNKET binding proteins are set forth in Table 15 below.

TABLE 15
Amino Acid Sequences of CEACAM5 TriNKET Binding Proteins

| | |
|---|---|
| A49 MI Heavy Chain | EVQLVESGGGLVKPGGSLRLSCAASGFTFSSYSMNWVRQAPG |
| | KGLEWVSSISSSSSYIYYADSVKGRFTISRDNAKNSLYLQMNS |
| | LRAEDTAVYYCARGAPIGAAAGWFDPWGQGTLVTVSSASTK |
| | GPSVFPLAPSSKSTSGGTAALGCLVKDYFPEPVTVSWNSGALT |
| | SGVHTFPAVLQSSGLYSLSSVVTVPSSSLGTQTYICNVNHKPS |
| | NTKVDKKVEPKSCDKTHTCPPCPAPELLGGPSVFLFPPKPKDT |
| | LMISRTPEVTCVVVDVSHEDPEVKFNWYVDGVEVHNAKTKP |
| | REEQYNSTYRVVSVLTVLHQDWLNGKEYKCKVSNKALPAPI |
| | EKTISKAKGQPREPQVCTLPPSRDELTENQVSLTCLVKGFYPS |
| | DIAVEWESNGQPENNYKTTPPVLDSDGSFFLYSWLTVDKSRW |
| | QQGNVFSCSVMHEALHNHYTQKSLSLSPG (SEQ ID NO: 549) |
| A49 MI scFv | DIQMTQSPSSVSASVGDRVTITCRASQGISSWLAWYQQKPGK |
| | APKLLIYAASSLQSGVPSRFSGSGSGTDFTLTISSLQPEDFATY |
| | YCQQGVSFPRTFGCGTKVEIKGGGGSGGGGSGGGGSGGGGSE |
| | VQLVESGGGLVKPGGSLRLSCAASGFTFSSYSMNWVRQAPG |
| | KCLEWVSSISSSSSYIYYADSVKGRFTISRDNAKNSLYLQMNS |
| | LRAEDTAVYYCARGAPIGAAAGWFDPWGQGTLVTVSS (SEQ |
| | ID NO: 700) |
| A49 MI VH | EVQLVESGGGLVKPGGSLRLSCAASGFTFSSYSMNWVRQAPG |
| and CDRs | KGLEWVSSISSSSSYIYYADSVKGRFTISRDNAKNSLYLQMNS |
| | LRAEDTAVYYCARGAPIGAAAGWFDPWGQGTLVTVSS (SEQ |
| | ID NO: 508) |
| | CDR1: GFTFSSY (SEQ ID NO: 2) (Chothia) or |
| | SYSMN (SEQ ID NO: 495) (Kabat) |
| | CDR2: SSSSSY (SEQ ID NO: 701) (Chothia) or |
| | SISSSSSYIYYADSVKG (SEQ ID NO: 496) (Kabat) |
| | CDR3: GAPIGAAAGWFDP (SEQ ID NO: 510) (Chothia) or |
| | GAPIGAAAGWFDP (SEQ ID NO: 510) (Kabat) |
| A49 VL | DIQMTQSPSSVSASVGDRVTITCRASQGISSWLAWYQQKPGK |
| and CDRs | APKLLIYAASSLQSGVPSRFSGSGSGTDFTLTISSLQPEDFATY |
| | YCQQGVSFPRTFGGGTKVEIK (SEQ ID NO: 493) |
| | CDR1: RASQGISSWLA (SEQ ID NO: 530) (Chothia) or |
| | RASQGISSWLA (SEQ ID NO: 530) (Kabat) |
| | CDR2: AASSLQS (SEQ ID NO: 224) (Chothia) or |
| | AASSLQS (SEQ ID NO: 224) (Kabat) |
| | CDR3: QQGVSFPRT (SEQ ID NO: 499) (Chothia) or |
| | QQGVSFPRT (SEQ ID NO: 499) (Kabat) |
| AB0264 Heavy Chain | DIVMTQSPDSLAVSLGERATINCKSSHSLLYGNFQNNYLAWY |
| | QQKPGQPPKLLIYWASTRESGVPDRFSGSGSGTDFTLTISSLQA |
| | EDVAVYYCQQYYSYPYTFGCGTKLEIKGGGGSGGGGSGGGG |
| | SGGGGSQVQLVQSGAEVKKPGASVKVSCKASGYTFTAYTIH |
| | WVRQAPGQCLEWMGYINPSSGYTEYNQKFKDRTTLTADTSIP |
| | TAYMELSRLRSDDTAVYYCTREGGLLWFDYWGQGTLVTVSS |
| | GSDKTHTCPPCPAPELLGGPSVFLFPPKPKDTLMISRTPEVTCV |
| | VVDVSHEDPEVKFNWYVDGVEVHNAKTKPREEQYNSTYRV |
| | VSVLTVLHQDWLNGKEYKCKVSNKALPAPIEKTISKAKGQPR |
| | EPRVYTLPPCRDELTKNQVSLTCLVKGFYPSDIAVEWESNGQP |
| | ENNYKTTPPVLVSDGSFTLYSKLTVDKSRWQQGNVFSCSVM |
| | HEALHNHYTQKSLSLSPG (SEQ ID NO: 702) |
| AB0264 scFv | DIVMTQSPDSLAVSLGERATINCKSSHSLLYGNFQNNYLAWY |
| (VL-VH) | QQKPGQPPKLLIYWASTRESGVPDRFSGSGSGTDFTLTISSLQA |
| | EDVAVYYCQQYYSYPYTFGCGTKLEIKGGGGSGGGGSGGGG |
| | SGGGGSQVQLVQSGAEVKKPGASVKVSCKASGYTFTAYTIH |
| | WVRQAPGQCLEWMGYINPSSGYTEYNQKFKDRTTLTADTSIP |
| | TAYMELSRLRSDDTAVYYCTREGGLLWFDYWGQGTLVTVSS |
| | (SEQ ID NO: 703) |
| AB0264 VH | QVQLVQSGAEVKKPGASVKVSCKASGYTFTAYTIHWVRQAP |
| and CDRs | GQCLEWMGYINPSSGYTEYNQKFKDRTTLTADTSIPTAYMEL |
| | SRLRSDDTAVYYCTREGGLLWFDYWGQGTLVTVSS (SEQ ID |
| | NO: 704) |
| | CDR1: GYTFTAY (SEQ ID NO: 101) (Chothia) or |
| | AYTIH (SEQ ID NO: 102) (Kabat) |
| | CDR2: NPSSGY (SEQ ID NO: 103) (Chothia) or |
| | YINPSSGYTEYNQKFKD (SEQ ID NO: 104) (Kabat) |
| | CDR3: EGGLLWFDY (SEQ ID NO: 105) (Chothia) or |
| | EGGLLWFDY (SEQ ID NO: 105) (Kabat) |
| AB0264 VL | DIVMTQSPDSLAVSLGERATINCKSSHSLLYGNFQNNYLAWY |
| and CDRs | QQKPGQPPKLLIYWASTRESGVPDRFSGSGSGTDFTLTISSLQA |
| | EDVAVYYCQQYYSYPYTFGCGTKLEIK (SEQ ID NO: 705) |
| | CDR1: KSSHSLLYGNFQNNYLA (SEQ ID NO: 107) (Chothia) or |
| | KSSHSLLYGNFQNNYLA (SEQ ID NO: 107) (Kabat) |
| | CDR2: WASTRES (SEQ ID NO: 108) (Chothia) or |
| | WASTRES (SEQ ID NO: 108) (Kabat) |
| | CDR3: QQYYSYPYT (SEQ ID NO: 109) (Chothia) or |
| | QQYYSYPYT (SEQ ID NO: 109) (Kabat) |
| AB0411 Heavy Chain | DIQLTQSPATLSVSPGERATLSCRASQSVSSSYLAWYQQKPGQ |
| | APRLLIYGASSRATGIPDRFSGSGSGTDFTLTISRLEPEDFAVY |
| | YCQQYNNWPLTFGCGTKVEIKGGGGSGGGGSGGGGSGGGGS |
| | QVQLVQSGGGLVQPGGSLRLSCAASGFTFTSYAMSWVRQAP |
| | GKCLEWVSAISGTGDSTFYADSVKGRFTFSRDNSKNTLYLQM |
| | NSLRAEDTAVYYCAKDLGWLQYGLFDYWGQGTLVTVSSGS |
| | DKTHTCPPCPAPELLGGPSVFLFPPKPKDTLMISRTPEVTCVVV |
| | DVSHEDPEVKFNWYVDGVEVHNAKTKPREEQYNSTYRVVSV |
| | LTVLHQDWLNGKEYKCKVSNKALPAPIEKTISKAKGQPREPR |
| | VYTLPPCRDELTKNQVSLTCLVKGFYPSDIAVEWESNGQPEN |
| | NYKTTPPVLVSDGSFTLYSKLTVDKSRWQQGNVFSCSVMHE |
| | ALHNHYTQKSLSLSPG (SEQ ID NO: 706) |
| AB0411 scFv | DIQLTQSPATLSVSPGERATLSCRASQSVSSSYLAWYQQKPGQ |
| (VL-VH) | APRLLIYGASSRATGIPDRFSGSGSGTDFTLTISRLEPEDFAVY |
| | YCQQYNNWPLTFGCGTKVEIKGGGGSGGGGSGGGGSGGGGS |
| | QVQLVQSGGGLVQPGGSLRLSCAASGFTFTSYAMSWVRQAP |
| | GKCLEWVSAISGTGDSTFYADSVKGRFTFSRDNSKNTLYLQM |
| | NSLRAEDTAVYYCAKDLGWLQYGLFDYWGQGTLVTVSS |
| | (SEQ ID NO: 707) |
| AB0411 VH | QVQLVQSGGGLVQPGGSLRLSCAASGFTFTSYAMSWVRQAP |
| and CDRs | GKCLEWVSAISGTGDSTFYADSVKGRFTFSRDNSKNTLYLQM |
| | NSLRAEDTAVYYCAKDLGWLQYGLFDYWGQGTLVTVSS |
| | (SEQ ID NO: 708) |
| | CDR1: GFTFTSY (SEQ ID NO: 35) (Chothia) or |
| | SYAMS (SEQ ID NO: 3) (Kabat) |
| | CDR2: SGTGDS (SEQ ID NO: 36) (Chothia) or |
| | AISGTGDSTFYADSVKG (SEQ ID NO: 37) (Kabat) |
| | CDR3: DLGWLQYGLFDY (SEQ ID NO: 38) (Chothia) or |
| | DLGWLQYGLFDY (SEQ ID NO: 38) (Kabat) |
| AB0411 VL | DIQLTQSPATLSVSPGERATLSCRASQSVSSSYLAWYQQKPGQ |
| and CDRs | APRLLIYGASSRATGIPDRFSGSGSGTDFTLTISRLEPEDFAVY |
| | YCQQYNNWPLTFGCGTKVEIK (SEQ ID NO: 591) |
| | CDR1: RASQSVSSSYLA (SEQ ID NO: 40) (Chothia) or |
| | RASQSVSSSYLA (SEQ ID NO: 40) (Kabat) |
| | CDR2: GASSRAT (SEQ ID NO: 41) (Chothia) or |
| | GASSRAT (SEQ ID NO: 41) (Kabat) |
| | CDR3: QQYNNWPLT (SEQ ID NO: 42) (Chothia) or |
| | QQYNNWPLT (SEQ ID NO: 42) (Kabat) |
| AB0466 Heavy Chain | DIQMTQSPSTLSASVGDRVTITCWASQSISSWLAWYQQKPGK |
| | APKLLIYKASSLESGVPSRFSGSGSGTEFTLTISSLQPDDFATYY |
| | CQQYNAYSYTFGCGTKLEIKGGGGSGGGGSGGGGSGGGGSQ |
| | VQLVQSGGGLVQPGGSLRLSCAASGFTFSSYAMSWVRQAPG |
| | KCLEWVSAIFSSGGSTYYADSVKGRFTVSRDNSKNTLYLQMN |
| | SLRAEDTALYYCAKDLGGYNYGLFDYWGQGTLVTVSSGSDK |
| | THTCPPCPAPELLGGPSVFLFPPKPKDTLMISRTPEVTCVVVDV |
| | SHEDPEVKFNWYVDGVEVHNAKTKPREEQYNSTYRVVSVLT |
| | VLHQDWLNGKEYKCKVSNKALPAPIEKTISKAKGQPREPRVY |
| | TLPPCRDELTKNQVSLTCLVKGFYPSDIAVEWESNGQPENNY |
| | KTTPPVLVSDGSFTLYSKLTVDKSRWQQGNVFSCSVMHEALH |
| | NHYTQKSLSLSPG (SEQ ID NO: 709) |
| AB0466 scFv | DIQMTQSPSTLSASVGDRVTITCWASQSISSWLAWYQQKPGK |
| (VL-VH) | APKLLIYKASSLESGVPSRFSGSGSGTEFTLTISSLQPDDFATYY |
| | CQQYNAYSYTFGCGTKLEIKGGGGSGGGGSGGGGSGGGGSQ |
| | VQLVQSGGGLVQPGGSLRLSCAASGFTFSSYAMSWVRQAPG |
| | KCLEWVSAIFSSGGSTYYADSVKGRFTVSRDNSKNTLYLQMN |
| | SLRAEDTALYYCAKDLGGYNYGLFDYWGQGTLVTVSS (SEQ |
| | ID NO: 710) |
| AB0466 VH | QVQLVQSGGGLVQPGGSLRLSCAASGFTFTSYAMSWVRQAP |
| and CDRs | GKCLEWVSAISGTGDSTFYADSVKGRFTFSRDNSKNTLYLQM |
| | NSLRAEDTAVYYCAKDLGWLQYGLFDYWGQGTLVTVSS |
| | (SEQ ID NO: 711) |
| | CDR1: GFTFSSY (SEQ ID NO: 2) (Chothia) or |
| | SYAMS (SEQ ID NO: 3) (Kabat) |
| | CDR2: FSSGGS (SEQ ID NO: 717) (Chothia) or |
| | AIFSSGGSTYYADSVKG (SEQ ID NO: 718) (Kabat) |
| | CDR3: DLGGYNYGLFDY (SEQ ID NO: 6) (Chothia) or |
| | DLGGYNYGLFDY (SEQ ID NO: 6) (Kabat) |
| AB0466 VL | DIQLTQSPATLSVSPGERATLSCRASQSVSSSYLAWYQQKPGQ |
| and CDRs | APRLLIYGASSRATGIPDRFSGSGSGTDFTLTISRLEPEDFAVY |
| | YCQQYNNWPLTFGCGTKVEIK (SEQ ID NO: 712) |
| | CDR1: WASQSISSWLA (SEQ ID NO: 7) (Chothia) or |
| | WASQSISSWLA (SEQ ID NO: 7) (Kabat) |
| | CDR2: KASSLES (SEQ ID NO: 8) (Chothia) or |
| | KASSLES (SEQ ID NO: 8) (Kabat) |
| | CDR3: QQYNAYSYT (SEQ ID NO: 27) (Chothia) or |
| | QQYNAYSYT (SEQ ID NO: 27) (Kabat) |
| AB0621 Heavy Chain | DIVMTQSPDSLAVSLGERATINCKSSHSLLYGNFQNNYLAWY |
| | QQKPGQPPKLLIYWASTRESGVPDRFSGSGSGTDFTLTISSLQA |
| | EDVAVYYCQQYYSYPYTFGCGTKLEIKGGGGSGGGGSGGGG |
| | SGGGGSQVQLVQSGAEVKKPGASVKVSCKASGYTFTAYTIH |
| | WVRQAPGQCLEWMGYINPSSGYTEYNQKFKDRTTLTADTSIS |
| | TAYMELSRLRSDDTAVYYCTREGGLLWFDYWGQGTLVTVSS |
| | GSDKTHTCPPCPAPELLGGPSVFLFPPKPKDTLMISRTPEVTCV |
| | VVDVSHEDPEVKFNWYVDGVEVHNAKTKPREEQYNSTYRV |
| | VSVLTVLHQDWLNGKEYKCKVSNKALPAPIEKTISKAKGQPR |
| | EPRVYTLPPCRDELTKNQVSLTCLVKGFYPSDIAVEWESNGQP |
| | ENNYKTTPPVLVSDGSFTLYSKLTVDKSRWQQGNVFSCSVM |
| | HEALHNHYTQKSLSLSPG (SEQ ID NO: 713) |
| AB0621 scFv | DIVMTQSPDSLAVSLGERATINCKSSHSLLYGNFQNNYLAWY |
| (VL-VH) | QQKPGQPPKLLIYWASTRESGVPDRFSGSGSGTDFTLTISSLQA |
| | EDVAVYYCQQYYSYPYTFGCGTKLEIKGGGGSGGGGSGGGG |
| | SGGGGSQVQLVQSGAEVKKPGASVKVSCKASGYTFTAYTIH |
| | WVRQAPGQCLEWMGYINPSSGYTEYNQKFKDRTTLTADTSIS |
| | TAYMELSRLRSDDTAVYYCTREGGLLWFDYWGQGTLVTVSS |
| | (SEQ ID NO: 714) |
| AB0621 VH | QVQLVQSGAEVKKPGASVKVSCKASGYTFTAYTIHWVRQAP |
| and CDRs | GQCLEWMGYINPSSGYTEYNQKFKDRTTLTADTSISTAYMEL |
| | SRLRSDDTAVYYCTREGGLLWFDYWGQGTLVTVSS (SEQ ID |
| | NO: 715) |
| | CDR1: GYTFTAY (SEQ ID NO: 101) (Chothia) or |
| | AYTIH (SEQ ID NO: 102) (Kabat) |
| | CDR2: NPSSGY (SEQ ID NO: 103) (Chothia) or |
| | YINPSSGYTEYNQKFKD (SEQ ID NO: 104) (Kabat) |
| | CDR3: EGGLLWFDY (SEQ ID NO: 105) (Chothia) or |
| | EGGLLWFDY (SEQ ID NO: 105) (Kabat) |
| AB0621 VL | DIVMTQSPDSLAVSLGERATINCKSSHSLLYGNFQNNYLAWY |
| and CDRs | QQKPGQPPKLLIYWASTRESGVPDRFSGSGSGTDFTLTISSLQA |
| | EDVAVYYCQQYYSYPYTFGCGTKLEIK (SEQ ID NO: 716) |
| | CDR1: KSSHSLLYGNFQNNYLA (SEQ ID NO: 107) (Chothia) or |
| | KSSHSLLYGNFQNNYLA (SEQ ID NO: 107) (Kabat) |
| | CDR2: WASTRES (SEQ ID NO: 108) (Chothia) or |
| | WASTRES (SEQ ID NO: 108) (Kabat) |
| | CDR3: QQYYSYPYT (SEQ ID NO: 109) (Chothia) or |
| | QQYYSYPYT (SEQ ID NO: 109) (Kabat) |

### Production and Purification of F3′ CEACAM5 TriNKET Multispecific Binding Proteins

F3′ CEACAM5 TriNKET molecules were expressed transiently in Chinese Hamster Ovary (CHO cells) using various DNA ratios for the various protein chains. Supernatant containing the expressed molecules were captured from culture supernatant by overnight incubation with Protein A chromatography resin using established methods. Bound TriNKET molecules were eluted from the Protein A resin with 0.1M glycine, pH 3.5.

Following adjustment to pH 7.0, the sample eluate was passed through an anion exchange (AEX) resin and the flow-through was collected. The sample flow-through was then polished by cation-exchange (CEX) chromatography using traditional methods to yield a purity of >98%, as determined by capillary gel electrophoresis, analytical size exclusion chromatography, and intact mass by mass spectrometry.

### Example 13. Binding of CEACAM5 TriNKETs to Target Proteins

### Binding to Human and Cynomolgus CEACAM5 at pH 6.0 and 7.4

To determine the affinity and cross-reactivity of CEACAM5 TriNKET molecules, AB0411, AB0466, or AB0621 TriNKETs were captured using an anti-human Fc capture antibody (Cytiva, #BR100839) immobilized onto a Biacore CM5 standard surface sensor chip following manufacturer instructions. Human or cynomolgus CEACAM5 were titrated over captured AB0411, AB0466, and AB0621 as 2-fold serial dilutions starting from 300 nM. Association was monitored for 300 sec and dissociation was monitored for 600 sec. The assay was run at 37° C. at pH 7.4 and 6.0. The surface of the CM5 chip was regenerated with 10 mM glycine pH 1.5 for 20 sec at 100 μL/min.

Results indicate that AB0411 and AB0466 bind to human but not cynomolgus CEACAM5 with AB0411 showing higher affinity than AB0466. AB0621 binds both human and cynomolgus CEACAM5 at both pH 7.4 and 6.0 at comparable affinities to AB0466 with a slight affinity increase at the lower pH. AB0264 showed comparable affinities to AB0621 but against the K398 allele of human CEACAM5. The results of the in vitro binding assays are show in Table 16.

TABLE 16
CEACAM5 TriNKET In Vitro Binding Assays

| | | Human CEACAM5 (E398) | | | Cynomolgus CEACAM5 | | |
|---|---|---|---|---|---|---|---|
| | 37° C. | ka (1/Ms) | kd (1/M) | KD (M) | ka (1/Ms) | kd (1/M) | KD (M) |
| AB0411 | pH 7.4 | 2.7E+05 | 4.3E−04 | 1.7E−09 | NB | NB | NB |
| | pH 6.0 | 3.1E+05 | 5.9E−04 | 1.9E−09 | NB | NB | NB |
| AB0466 | pH 7.4 | 1.9E+05 | 1.3E−03 | 7.2E−09 | NB | NB | NB |
| | pH 6.0 | 1.6E+05 | 2.5E−03 | 1.5E−08 | NB | NB | NB |
| AB0621 | pH 7.4 | 1.0E+05 | 1.7E−03 | 1.7E−08 | 6.1E+04 | 8.4E−04 | 1.4E−08 |
| | pH 6.0 | 1.4E+05 | 1.9E−03 | 1.4E−08 | 1.3E+05 | 8.1E−04 | 6.5E−09 |
| AB0264* | pH 7.4 | 9.1E+04 | 1.2E−03 | 1.3E−08 | ND | ND | ND |
| | pH 6.0 | ND | ND | ND | ND | ND | ND |

NB—No binding
ND—Not determined
*Used CEACAM5 (K398)

### CEACAM5 TriNKETs Bind to Distinct Domains of Human CEACAM5

To determine to which hCEACAM5 protein domain(s) the TriNKETs bound, different assay formats were run depending on whether the protein domain had a His-tag or was an Fc fusion. If the protein domain was an Fc fusion, AB0411, AB0466, AB0264, or AB0621 were captured using an anti-human Fab antibody (Cytiva, #28958325) immobilized onto a CM5 chip. The various hCEACAM5 protein domains (N-term, A1-B1, A2, B2, A3, or B3) were titrated over the captured TriNKETs at 50 μL/min for 180 sec of association and 300 sec of dissociation. The assay was run at 25° C. in HBS-EP+ running buffer, pH 7.4. The surface of the CM5 chip was regenerated using 10 mL glycine pH 2.1.

If the protein domain had a His-tag, AB0411, AB0466, AB0264, or AB0621 were captured using an anti-human Fc antibody (Cytiva) immobilized onto a CM5 chip. The various hCEACAM5 protein domains were titrated over the captured antibody at 50 μL/min for 180 sec of association and 300 sec of dissociation. The assay was run at 37° C. in HBS-EP+ running buffer, pH 7.4. The surface of the CM5 chip was regenerated using 3M MgCl₂.

Results showed that the TriNKETs bound only to the A1-B1 domain, consistent with hydrogen-deuterium exchange mass spectrometry (HDX-MS) epitope mapping data (not shown). The affinity for A1-B1 domain is comparable to that for the ectodomain of human CEACAM5. None of the TriNKETs showed binding to A2-B2 or A3-B3 domain. The results of the domain binding assays are show in Table 17.

TABLE 17
Human CEACAM5 Protein Domain Binding Assays

| | Format | | ka (1/Ms) | kd (1/M) | KD (M) |
|---|---|---|---|---|---|
| | | | AB0411 | | |
| CEACAM5 Nterm IgV-His | hFc capt | 37° C. | 1.4E+06 | 4.2E−04 | 3.1E−10 |
| CEACAM5 N-A1-B1-His | hFc capt | 37° C. | 3.6E+05 | 4.3E−04 | 1.2E−09 |
| CEACAM5 N-A1-B1-His | Fab capt | 25° C. | 3.0E+05 | 2.8E−04 | 9.5E−10 |
| CEACAM5 A1-B1-Fc | Fab capt | 25° C. | | | |
| | | | AB0466 | | |
| CEACAM5 Nterm IgV-His | hFc capt | 37° C. | 1.1E+06 | 1.5E−03 | 1.4E−09 |
| CEACAM5 N-A1-B1-His | hFc capt | 37° C. | 2.4E+05 | 1.3E−03 | 5.5E−09 |
| CEACAM5 N-A1-B1-His | Fab capt | 25° C. | 2.8E+05 | 5.8E−04 | 2.1E−09 |
| CEACAM5 A1-B1-Fc | Fab capt | 25° C. | | | |
| | | | AB0621 | | |
| CEACAM5 Nterm IgV-His | hFc capt | 37° C. | NB | NB | NB |
| CEACAM5 N-A1-B1-His | hFc capt | 37° C. | 1.5E+05 | 1.7E−03 | 1.1E−08 |
| CEACAM5 N-A1-B1-His | Fab capt | 25° C. | 3.5E+05 | 5.9E−04 | 1.8E−09 |
| CEACAM5 A1-B1-Fc | Fab capt | 25° C. | 2.5E+05 | 3.6E−04 | 1.5E−09 |
| | | | AB0264 | | |
| CEACAM5 Nterm IgV-His | hFc capt | 37° C. | | | |
| CEACAM5 N-A1-B1-His | hFc capt | 37° C. | | | |
| CEACAM5 N-A1-B1-His | Fab capt | 25° C. | 4.1E+05 | 4.5E−04 | 1.1E−09 |
| CEACAM5 A1-B1-Fc | Fab capt | 25° C. | 2.6E+05 | 2.7E−04 | 1.0E−09 |

### Binding of CEACAM5 TriNKETs to Human CEACAM5 SSNPs

Several notable single nucleotide polymorphisms (SNPs) of human CEACAM5 were identified and these were produced and assessed for binding to AB0411, AB0466, and AB0621 using a Biacore Surface Plasmon Resonance (SPR) assay developed for the major CEACAM5 SNP E398. Fold affinity loss (KD/KD(Ref)) was calculated for comparison. As shown in Table 18 below, AB0621 showed up to ˜2-fold affinity loss, AB0411 showed up to ˜5-fold affinity loss, and AB0466 showed up to ˜9-fold affinity loss against various SNPs.

TABLE 18
hCEACAM5 Domain In Vitro Binding Assays

| | | | | |
|---|---|---|---|---|
| pH 7.4, 37° C. | ka (1/Ms) | kd (1/M) | KD (M) | KD/KD(Ref) |

AB0411

| | | | | |
|---|---|---|---|---|
| E398 (Ref) | 2.7E+05 | 4.3E−04 | 1.7E−09 | 1.0 |
| K398 | 2.7E+05 | 3.9E−04 | 1.4E−09 | 0.9 |
| I80V V83A I112V I113T | 2.6E+05 | 1.7E−03 | 6.4E−09 | 3.9 |
| V83A | 1.8E+05 | 1.5E−03 | 8.2E−09 | 4.9 |
| I112V | 1.9E+05 | 3.7E−04 | 2.0E−09 | 1.2 |
| I113T | 2.1E+05 | 4.0E−04 | 1.9E−09 | 1.1 |

AB0466

| | | | | |
|---|---|---|---|---|
| E398 (Ref) | 1.9E+05 | 1.3E−03 | 7.2E−09 | 1.0 |
| K398 | 2.2E+05 | 1.4E−03 | 6.3E−09 | 0.9 |
| I80V V83A I112V I113T | 5.9E+05 | 3.1E−02 | 6.1E−08 | 8.5 |
| V83A | 1.4E+05 | 9.0E−03 | 6.6E−08 | 9.3 |
| I112V | 1.3E+05 | 1.5E−03 | 1.1E−08 | 1.6 |
| I113T | 1.6E+05 | 1.3E−03 | 8.4E−09 | 1.2 |

AB0621

| | | | | |
|---|---|---|---|---|
| E398 (Ref) | 1.0E+05 | 1.7E−03 | 1.7E−08 | 1.0 |
| K398 | 8.6E+04 | 1.6E−03 | 1.9E−08 | 1.1 |
| I80V V83A I112V I113T | 6.4E+04 | 1.4E−03 | 2.2E−08 | 1.3 |
| V83A | 4.4E+04 | 1.5E−03 | 3.4E−08 | 2.0 |
| I112V | 4.2E+04 | 1.6E−03 | 3.8E−08 | 2.3 |
| I113T | 4.7E+04 | 1.4E−03 | 2.9E−08 | 1.7 |

### Binding of CEACAM5 TriNKETs to Human and Cynomolgus CEACAM1,6,8

Binding to human and cynomolgus CEACAM1, 6, and 8 was assessed by SPR using the same protocol as for CEACAM5 but using up to 600 and 1200 nM for titration of each protein. There were no detectable binding signals at these concentrations for AB0621. In contrast, reproducible weak binding signals were detected for AB0411 and AB0466, but these two TriNKETs also bound to different epitope(s) on the N-terminus of CEACAM5. Typical weak binding sensorgrams for AB0411 and AB0466 against human CEACAM1 and 6 indicated weak but notable signals compared to AB0621. Steady-state approximations did not reach sufficiently high concentration to approach saturation for reliable KD determinations but KD estimates are likely to be values greater than about half the maximum analyte concentrations used. The results of the CEACAM1, 6, and 8 binding assays are show in Table 19.

TABLE 19
CEACAM1, 6, 8 Binding Assays

| pH 7.4, 37° C. | AB0411 | AB0466 | AB0621 | AB0264 |
|---|---|---|---|---|
| Human CEACAM1 | Weak | Weak | NB | NB |
| Human CEACAM6 | Weak | NB | NB | NB |
| Human CEACAM8 | Weak | | | |
| Cyno CEACAM1 | Weak | | NB | |
| Cyno CEACAM6 | NB | | NB | |
| Cyno CEACAM8 | NB | | NB | |

### Concomitant Binding to CEACAM5, NKG2D, and CD16 Target Proteins

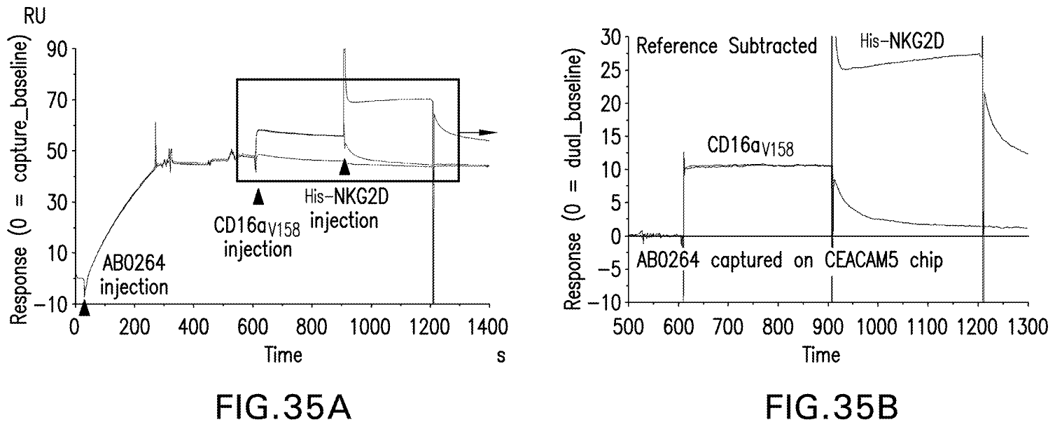

Concomitant binding of the three arms of AB0264 (which differs from AB0621 only in having a proline at position 77 instead of a serine) was demonstrated using SPR on a CM5 chip immobilized with human CEACAM5. This surface was used to first stably capture AB0264 onto the chip surface. FIG. 35A and FIG. 35B demonstrate that subsequent injections of saturating levels (2 μM) of CD16A (V158/V176) alone followed by a premix of 2 μM CD16A and 2 μM NKG2D-His demonstrated a stepwise binding signal indicative of a formation of a heterotetrameric complex on the chip surface. The ratio of binding signals (which are proportional to MWs) was approximately 5:1:1 which is consistent with a 1:1:1 molar stoichiometry of complex bound on the CEACAM5 surface.

### Binding to NKG2D at pH 7.4 and 6.0

Binding to NKG2D was assessed by SPR using a mouse Fc capture kit (Cytiva, #BR100838) immobilized onto a CM5 chip. Human and cynomolgus NKG2D fused to a mouse Fc were captured and AB0411, AB0466, or AB0621 was titrated from 600 nM. Results shown in Table 20 below demonstrate comparable affinities for all three TriNKETs against human and cyno NKG2D.

TABLE 20
NKG2D Binding Assays

| | 37° C. | NKG2D-mFc | | | Cyno NKG2D-mFc | | |
|---|---|---|---|---|---|---|---|
| AB0411 | pH 7.4 | 2.5E+05 | 1.2E—01 | 4.9E—07 | 2.6E+05 | 1.5E—01 | 5.9E—07 |
| | pH 6.0 | 2.8E+05 | 1.3E—01 | 4.9E—07 | 2.7E+05 | 1.4E—01 | 5.0E—07 |
| AB0466 | pH 7.4 | 2.4E+05 | 1.6E—01 | 6.8E—07 | 2.3E+05 | 1.8E—01 | 7.5E—07 |
| | pH 6.0 | 2.4E+05 | 1.2E—01 | 4.8E—07 | 3.1E+05 | 1.1E—01 | 4.2E—07 |
| AB0621 | pH 7.4 | 3.2E+05 | 1.4E—01 | 5.7E—07 | 3.2E+05 | 1.2E—01 | 5.2E—07 |
| | pH 6.0 | 2.4E+05 | 5.6E—02 | 2.4E—07 | 1.5E+05 | 4.5E—02 | 3.0E—07 |

### Binding to CD16A (FcγR3a)

Binding to human and cynomolgus CD16A was assessed using biotinylated CD16A captured on a streptavidin (SA) sensor chip (Cytiva, #BR100531). AB0411, AB0466, or AB0621 was titrated from 1500 nM at 25° C. at 30 μL/min for 150 sec association followed by 300 sec dissociation. The surface of the chip was regenerated with 2 mM sodium hydroxide for 5 sec at 30 μL/min. Running buffer was HBS-EP+. As shown in Table 21 below, the affinities were comparable among all TriNKETs. The V158 isoform bound ˜2- to 3-fold tighter than the F158 isoform as expected. Cyno FcγRIIIA showed slightly tighter binding than human FcγRIIIA V158.

TABLE 21
CD16A (FcγR3a) Binding Assays

| | 25° C. | FcγRIIIA V158 | | |
|---|---|---|---|---|
| AB0411 | pH 7.4 | 2.3E+05 | 2.9E−02 | 1.2E−07 |
| | pH 6.0 | 8.3E+05 | 2.9E−02 | 3.6E−08 |
| AB0466 | pH 7.4 | 3.0E+05 | 7.8E−02 | 2.6E−07 |
| | pH 6.0 | 8.2E+05 | 3.8E−02 | 4.7E−08 |
| AB0621 | pH 7.4 | 2.6E+05 | 7.2E−02 | 2.8E−07 |
| | pH 6.0 | 6.9E+05 | 4.0E−02 | 5.8E−08 |
| | 25° C. | FcγRIIIA F158 | | |
| AB0411 | pH 7.4 | 2.2E+05 | 8.6E−02 | 3.7E−07 |
| | pH 6.0 | ND | ND | ND |
| AB0466 | pH 7.4 | 3.1E+05 | 2.1E−01 | 6.9E−07 |
| | pH 6.0 | ND | ND | ND |
| AB0621 | pH 7.4 | 3.0E+05 | 1.9E−01 | 6.4E−07 |
| | pH 6.0 | ND | ND | ND |
| | 25° C. | Cyno FcγRIIIA | | |
| AB0411 | pH 7.4 | 7.3E+05 | 7.5E−02 | 1.0E−07 |
| | pH 6.0 | 8.9E+08 | 1.3E+01 | 1.6E−08 |
| AB0466 | pH 7.4 | 7.9E+05 | 1.1E−01 | 1.4E−07 |
| | pH 6.0 | 2.4E+06 | 4.7E−02 | 1.9E−08 |
| AB0621 | pH 7.4 | 7.2E+05 | 1.1E−01 | 1.6E−07 |
| | pH 6.0 | 9.7E+08 | 2.2E+01 | 2.5E−08 |

Binding of AB0621 to human FcγRs, excluding CD16a (FcγR3a)

AB0621 was captured onto a Protein A chip (Cytiva, #29127556). Human FcγRs were titrated over captured AB0621 as 3-fold serial dilutions starting from 300 nM for FcγRI, 1000 nM for FcγRIIA R131 and H131, and 3000 nM for FcγRIIB and FcγRIIIB. Association was monitored for 120 sec and dissociation was monitored for 180 sec. Assays were run at 25° C. at pH 7.4. The surface of the chip was regenerated with 10 mM glycine pH 1.5 for 20 sec at 30 μL/min. Results shown in Table 22 demonstrate that AB0621 binds to FcγRs with affinities comparable to those of a typical IgG1 antibody.

TABLE 22
FcγRs Binding Assays

| AB0621, pH | ka | kd | KD | KD/KD |
|---|---|---|---|---|
| 7.4, 25° C. | (1/Ms) | (1/M) | (M) | ref |
| FcγRI | 2.6E+06 | 3.6E−02 | 1.4E−08 | 0.64 |
| FcγRIIA H131 | — | — | 6.0E−07 | 0.68 |
| FcγRIIA R131 | — | — | 3.3E−07 | 0.68 |
| FcγRIIB | — | — | 3.3E−06 | 0.94 |
| FcγRIIIB | — | — | no binding | n/a |

### AB0621 Binding to Human FcRn

Anti-kappa light chain antibody was immobilized onto a CM5 chip following a standard amine-coupling protocol. AB0621 was captured onto the chip and human or cynomolgus FcRn was titrated from 2000 nM as 2-fold serial dilutions. Association was monitored for 120 sec and dissociation was monitored for 180 sec. Assays were run at 25° C. at both pH 7.4 and pH 6.0. The surface of the chip was regenerated with two pulses of 10 mM glycine pH 1.5 for 20 sec at 30 μL/min followed by 10 mM NaOH for 20 sec at 30 μL/min.

Results shown in Table 23 demonstrate that AB0621 binds both human and cynomolgus FcRn at pH 6.0 with comparable affinities, and that AB0621 does not bind to both human and cynomolgus FcRn at pH 7.4 as expected.

TABLE 23
FcRn Binding Assays

| AB0621, 25° C. | pH | KD (M) |
|---|---|---|
| Human FcRn | pH 6.0 | 4.4E−07 |
| | pH 7.4 | no binding |
| Cyno FcRn | pH 6.0 | 3.1E−07 |
| | pH 7.4 | no binding |

### Example 14. CEACAM5 TriNKET Cell Binding Assays

### Quantitation of Cell Surface CEACAM5 Molecules by Flow Cytometry

Various cancer cell lines were diluted into FACS buffer and 200,000 cells of each cell type were seeded per well in duplicate into a 96-well plate for FACS staining. A monovalent murine Fc variant of the anti-CEACAM5 antibody labetuzumab was diluted to 200 nM in FACS buffer and used to resuspend the cells. The plate was incubated at 4° C. for 120 minutes, washed with FACS buffer, and resuspended with the secondary detection reagent from the commercially available receptor quantitation kit QIFIKIT from Agilent. The FITC-anti murine secondary detection reagent was diluted 1:50 into FACS buffer and incubated onto the cells at 4° C. for 60 minutes. Calibration beads were washed with FACS buffer, resuspended with the FITC-anti murine detection reagent as prepared for the cells, and incubated at 4° C. for 60 minutes.

The cells and beads were washed with FACS buffer, resuspended with 70 μl fixation buffer, and incubated at 4° C. for 20 minutes. The cells and beads were washed again with FACS buffer and data were acquired using the Thermo Fisher Attune NxT. Cells of interest were identified using FSC vs. SSC plot, and an appropriately shaped gate was drawn around the cells. Within the gated cells, doublet events were removed by viewing FSC-H vs. FSC-A plot. Within the single cell population, live cells were gated. Within the live gate, the MFI of each sample was calculated. The MFI of the cells and calibration beads were background subtracted using wells containing secondary detection reagent only and converted into log(MFI). The log(MFI) of the calibration beads was plotted against the log(Receptor number, as provided by the manufacturer) and was fitted using linear regression using GraphPad Prism. The log(MFI) of the cells was then used to interpolate the log(Receptor number) of the cells; these data are reported as antibody binding capacity (ABC), or antibodies bound per cell. A wide-range of expression was observed across the cancer cell lines, with the highest number of CEACAM5 molecules per cell seen in MKN-45 cells, as shown in Table 24.

TABLE 24
Number of CEACAM5 Molecules Per Cell

| | Cell Surface |
|---|---|
| Cell Line | CEACAM5 |
| MKN-45 | 460,335 |
| SK-CO-1 | 118,984 |
| ZR-75-30 | 80,569 |
| HPAF-II | 63,997 |
| LS-174T | 44,027 |
| LoVo | 35,055 |
| BxPC-3 | 21,691 |
| Kato-III | 12,638 |
| AsPC-1 | 8,503 |
| HT-29 | 660 |

### Quantitation of Binding of TriNKETs to CEACAM5 on Tumor Cells Lines

MKN-45 and HPAF-II human cancer cell lines were diluted into FACS buffer and 100,000 cells of each cell type were seeded per well in duplicate into 96-well plates for FACS staining. The cells were washed with PBS, incubated in a 1:2000 dilution of live/dead dye in PBS for 15 minutes, and then washed with FACS buffer. AB0264, AB0411, AB0466, and AB0621 were diluted into FACS buffer, and 50 μl of each diluted TriNKET was added to the cells. After incubation on ice for 30-120 minutes, the cells were washed with FACS buffer. Anti-human IgG-Fc secondary antibody was diluted into FACS buffer, and 50 μl was added per well for detection of the bound TriNKETs. The cells were incubated for 30-60 minutes on ice and then washed with FACS buffer. 50 μl of fixation buffer was added to each well and the cells were incubated for 10 minutes at room temperature. The cells were washed with FACS buffer and resuspended in FACS buffer for analysis with ThermoFisher Attune NxT, BD FACS Celesta SN #H66034400085, or BD FACS Celesta SN #H66034400160.

Cells of interest were identified using FSC vs. SSC plot and an appropriately shaped gate was drawn around the cells. Within the gated cells, doublet cells were removed by viewing FSC-H vs. FSC-A plot. Within the single cell population, live cells were gated. Within the live gate, the median fluorescence intensity (MFI) of each sample and the secondary-only control was calculated. Fold-over-background (FOB) was calculated as the ratio of test article MFI over secondary-only background MFI. Data were fit to a four-parameter non-linear regression curve using GraphPad Prism 7.0.

The binding potency (EC50) and the maximum loading (Max FOB) of the CEACAM5 TriNKETs to MKN-45 and HPAF-II human cancer cell lines is shown in Table 25. For AB0261, the binding potency was similar for MKN-45 and HPAF-II, with EC50s of 20.6 nM and 18.1 nM, respectively. Maximum loading was 51.32 and 24.92 FOB, respectively, consistent with the high and moderate expression of CEACAM5 on these cell lines.

TABLE 25
EC50 and Max FOB Values for TriNKETs
Binding to Human Cancer Cell Lines

| | |
|---|---|
| AB0264 | AB0621 |

| Cell Line | EC50 nM | Max FOB | EC50 nM | Max FOB |
|---|---|---|---|---|
| MKN-45 | 24.6 | 54.97 | 20.6 | 51.32 |
| HPAF-II | 20.3 | 27.95 | 18.1 | 24.92 |

| | |
|---|---|
| AB0411 | AB0466 |

| Cell Line | EC50 nM | Max FOB | EC50 nM | Max FOB |
|---|---|---|---|---|
| MKN-45 | 16.2 | 119.9 | 15.2 | 111.2 |
| HPAF-II | 8.36 | 54.94 | 7.61 | 49.01 |

Comparison of the Binding of TriNKETs and mAbs to CEACAM5 on Tumor Cells Lines

Using the methods described above, the binding of the CEACAM5 TriNKETs was compared with their respective corresponding monoclonal antibodies across five human cancer cell lines (Table 26). MKN-45 and SK-CO-1 cells were representative of higher CEACAM5 expression levels, while LoVo and BxPC-3 represented medium expression, and KATO-III represented low CEACAM5 expression. Similar binding patterns were seen across the five cell lines. AB0264 and AB0411 showed reduced EC50s compared to AB0755 and AB0509 respectively. AB0755 is a humanized mAb against CEACAM5 with Fab sequence corresponding to scFV anti-CEACAM5 present in AB0264. AB0509 is a humanized mAb against CEACAM5 with Fab sequence corresponding to scFV anti-CEACAM5 present in AB0411. However, both TriNKETs consistently loaded to a higher Max FOB than their corresponding mAbs.

TABLE 26
EC50 and Max FOB Values for TriNKETs
and Corresponding mAbs

| | |
|---|---|
| AB0264 | AB0757 |

| Cell Line | EC50 nM | Max FOB | EC50 nM | Max FOB |
|---|---|---|---|---|
| MKN-45 | 20.7 | 60 | 4.69 | 23 |
| SK-CO-1 | 104 | 42 | 5.84 | 14 |
| LoVo | 22.1 | 14 | 2.71 | 7 |
| BxPC-3 | 30.5 | 12 | 3.37 | 5 |
| Kato-III | 13.3 | 4 | 1.16 | 3 |

| | |
|---|---|
| AB0411 | AB0509 |

| Cell Line | EC50 nM | Max FOB | EC50 nM | Max FOB |
|---|---|---|---|---|
| MKN-45 | 9.05 | 107 | 9.05 | 107 |
| SK-CO-1 | 8.67 | 37 | 8.67 | 37 |
| LoVo | 3.25 | 19 | 3.25 | 19 |
| BxPC-3 | 4.28 | 19 | 4.28 | 19 |
| Kato-III | 1.17 | 5 | 1.17 | 5 |

### CEACAM5 TriNKET Binding to Ba/F3 Cells Expressing CEACAM Family Proteins

To examine the binding specificity of the TriNKETs to CEACAM5 compared to other CEACAM family members, Ba/F3 cells were engineered to express one of human CEACAM1, CEACAM6, CEACAM8, or cynomolgus CEACAM5. CEACAM5 TriNKETs were evaluated by flow cytometry starting at 1600 nM followed by 7 5-fold dilutions.

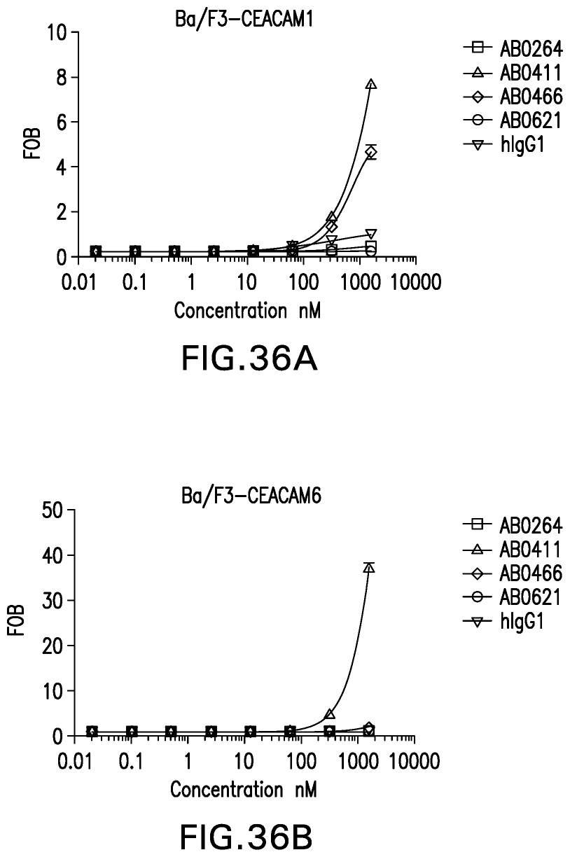

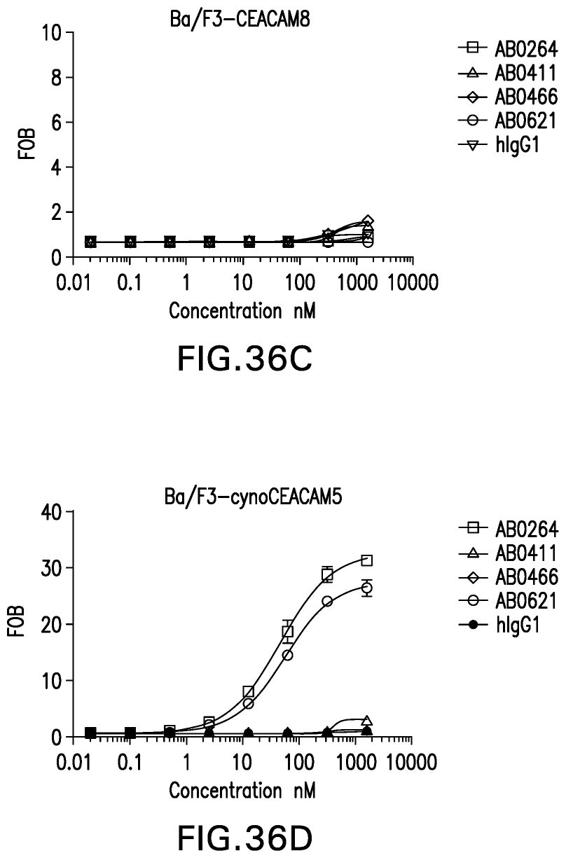

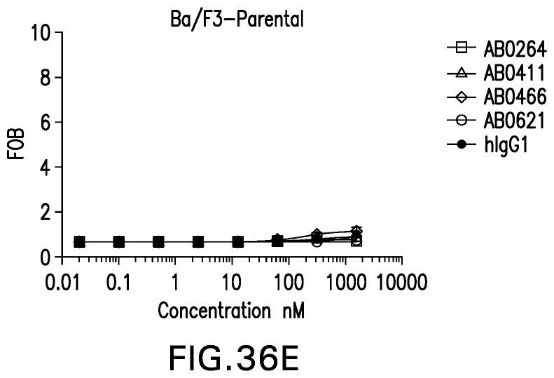

AB0264 and AB0621 bound to cynomolgus monkey CEACAM5, while AB0466 and AB0411 did not (FIG. 36D). AB0264 and AB0621 did not cross-react to Ba/F3 cells expressing either human CEACAM1, CEACAM6, or CEACAM8 (FIG. 36A-36C), while both AB0411 and AB0466 showed cross-reactive binding to Ba/F3 cell expressing human CEACAM11 (FIG. 36A), and AB0411 also showed cross-reactive binding to Ba/F3 cell expressing human CEACAM6 (FIG. 36B). CEACAM5 TriNKETs did not bind to parental BA/F3 cells which lacked expression of CEACAM family member proteins (FIG. 36E).

### Example 15. Cell-Based Cytotoxicity and Activity Assays

### Isolation and Preparation of Primary PBMCs and NK Cells

Human blood was obtained from Stanford Blood Bank or Biological Specialty Corporation (#225-11-04). Cynomolgus whole blood from three animals was obtained from BioIVT (#NHP01WBNHUZN). Both human and cynomolgus PBMCs were isolated by density gradient centrifugation. After purification, PBMCs were either used immediately or were frozen for later use. Human primary NK cells were purified by negative depletion using EasySep™ (StemCell, #17955) or RosetteSep™ (StemCell, #15065) following manufacturer's protocol. Alternatively, frozen NK cells were purchased from BioIVT (#HUMAN-HL65-U-200429). Primary NK cells were cultured in RPMI primary cell medium overnight before use in assays, e.g., in a DELFIA assay.

### Transduction of KHYG-1-CD16V Cells

KHYG-1 cells (DSMZ, #ACC-725) were transduced with a retrovirus to express human CD16a variant 158V (UniProt P08637). Cells were selected under puromycin and the resistant population positive for human CD16 was confirmed by FACS analysis. KHYG-1-CD16V cells were routinely maintained in RPMI medium at a density between 0.2×10⁶-1.0×10⁶/mL in the presence of 10 ng/mL recombinant human IL-2.

### Preparation of CD8⁺ T Cells

Frozen PBMCs were thawed and stimulated with 1 μg/mL ConA in culture media in 25 cm² flasks with 20-25×10⁶ cells per 10 ml per flask at 37° C. for 18 hours. ConA was then removed and PBMCs were cultured with 25 units/mL IL-2 in 25 cm² flasks at 37° C. for 4 days. CD8⁺ T cells were purified using a negative selection technique with magnetic beads (EasySep™ Human CD8⁺ T Cell Isolation Kit, StemCell), according to manufacturer's instructions. Finally, CD8⁺ T cells were cultured in media containing 10 ng/mL IL-15 at 100,000 cells/200 μL/well in 96-well round bottom plates at 37° C. for 6-10 days before use, e.g., in cytolysis assays. Human effector CD8⁺ T cells generated above were analyzed by flow cytometry for CD3+CD8⁺ cell purity as well as NKG2D and CD16 expression. CD8⁺ T cell activity was measured in the DELFIA cytotoxicity assay described below.

### DELFIA Cytotoxicity Assay

CEACAM-5 expressing target cancer cells were dissociated from culture vessels, pelleted, washed with 1×HBS, and resuspended in pre-warmed cell culture media at 10⁶ cells/mL. BATDA (bis(acetoxymethyl) 2,2′:6′,2″-terpyridine-6,6″-dicarboxylate) reagent was diluted 1:400 into the cell suspension. Cells were mixed and incubated at 37° C. with 5% CO₂ for 15-20 minutes. The labeled target cells were washed 3× with 1×HBS and resuspended into a final desired concentration in cell culture media.

Rested effector cells, such as human NK cells, KHYG1-CD16V, or activated CD8⁺ T cells, were removed from culture and pelleted, the cells were resuspended in RPMI primary cell culture media. TriNKETs were titrated in RPMI primary cell culture media. Assays were set up in a round bottom TC 96-well plate with a desired amount of labeled target cells, effector cells, and TriNKETs.

Control wells for background were prepared using 100 μl of the supernatant from pelleting labeled target cells and an additional 100 μl of RPMI primary cell culture media. Spontaneous release wells were prepared by adding 100 μl of labeled target cells to wells containing 100 μl of RPMI primary cell culture media. Maximum release wells were prepared by adding 100 μl of labeled target cells to wells containing 80 μl of RPMI primary cell culture media and 20 μl of 10% TritonX-100 solution. The assay plate was incubated at 37° C. with 5% CO₂ for 2-3 hours.

At the end of the assay, 20 μl of supernatant from each well was removed and transferred to a clean 96-well DELFIA assay plate. 200 μl of Europium solution was added to each well and further incubated at room temperature for 15 minutes at 250 RPM on a plate shaker. An HTRF cartridge in a SpectraMax i3x or Envision® was used to read the assay plate. The mean of the background samples was calculated and subtracted from the value from all sample wells. Calculation of specific lysis was performed using the following formula:

% Specific lysis=(sample−spontaneous)/(max−spontaneous)*100%.

### Potency of CEACAM5 TriNKETs in Cytotoxicity Assays Using NK Cells and Tumor Cell Lines

Human NK cells were rested overnight. The following day, rested NK cells were co-cultured with BATDA-labeled CEACAM5-expressing target cancer cells at 10:1 (SK-CO-1) or 5:1 ratio (LS-174T, ZR-75-30, and HPAF-II) for the DELFIA assay. Data were fit to a 4-parameter non-linear regression model to generate potency values.

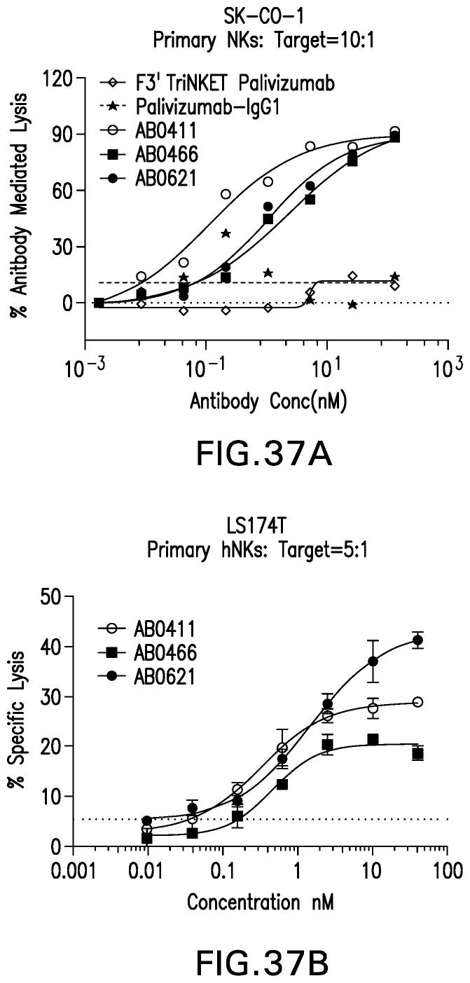

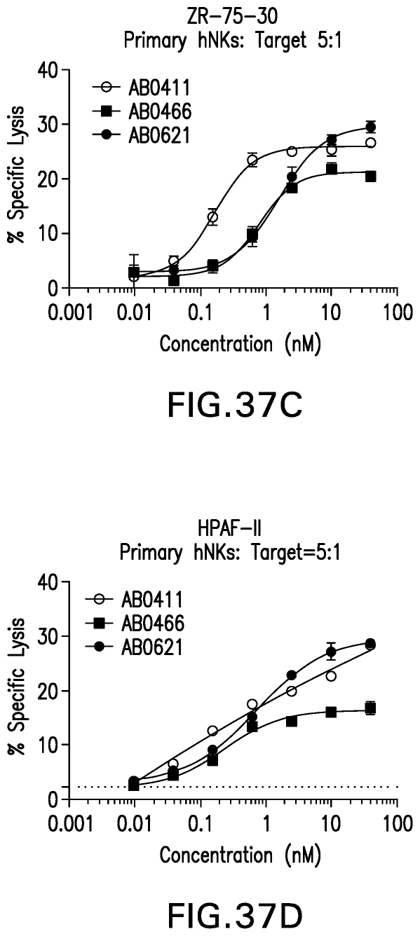

In a 2.5-hour short-term DELFIA assay using SK-CO-1 target cells, CEACAM5 TriNKETs elicited efficacious target cell lysis by rested primary NK cells from healthy human donors (FIG. 37A). Similar results for LS-174T, HPAF-II, and ZR075-30 are shown in FIGS. 37B-37D. An RSV-targeting TriNKET (F3′-TriNKET-palivizumab) and a human IgG1 isotype control (palivizumab-IgG1) resulted in minimal target cell death, suggesting the cytolytic effect was dependent on engagement of the anti-CEACAM5 arm to target cells. The concentration of AB0411, AB0466, and AB0621 required to produce half of its maximum killing (EC50) was 4.55 nM, 9.07 nM, and 1.02 nM, respectively (Table 27).

TABLE 27
Potency of CEACAM5 TriNKETs in the
Cytolysis of CEACAM5+ Cancer Cells

| | SK-CO-1 | | LS-174T | |
|---|---|---|---|---|
| | | Max | | Max |
| TriNKET | EC50 (nM) | Killing (%) | EC50 (nM) | Killing (%) |
| AB0411 | 4.55 | 84.0 | 0.32 | 28.91 |
| AB0466 | 9.07 | 75.1 | 0.46 | 20.54 |
| AB0621 | 1.02 | 80.9 | 1.87 | 42.63 |

| | |
|---|---|
| ZR-75-30 | HPAF-II |

| | EC50 | Max | EC50 | Max |
|---|---|---|---|---|
| TriNKET | (nM) | Killing (%) | (nM) | Killing (%) |
| AB0411 | 0.17 | 25.94 | Poor fit | 28.28 |
| AB0466 | 0.80 | 21.36 | 0.23 | 16.49 |
| AB0621 | 1.58 | 29.82 | 0.72 | 29.79 |

### AB0264 Enhances Activity of IL-2 Stimulated Human NK Cells

The ability of AB0264 to activate primary human NK cells was characterized. Purified frozen human NK cells were thawed and either rested or activated overnight in culture with IL-2. The following day, NK cells were co-cultured with labeled ZR-75-30 target cells for DELFIA assay. Dose-titrations of AB0264 or AB0755 (a humanized mAb against CEACAM5 with Fab sequence corresponding to scFV anti-CEACAM5 present in AB0264) were prepared starting at 50 nM and were added to the co-cultures of rested or activated human NK cells and ZR-75-30 target cells. Specific lysis was plotted against concentration and data were fit to a 4-parameter non-linear regression model to generate potency values.

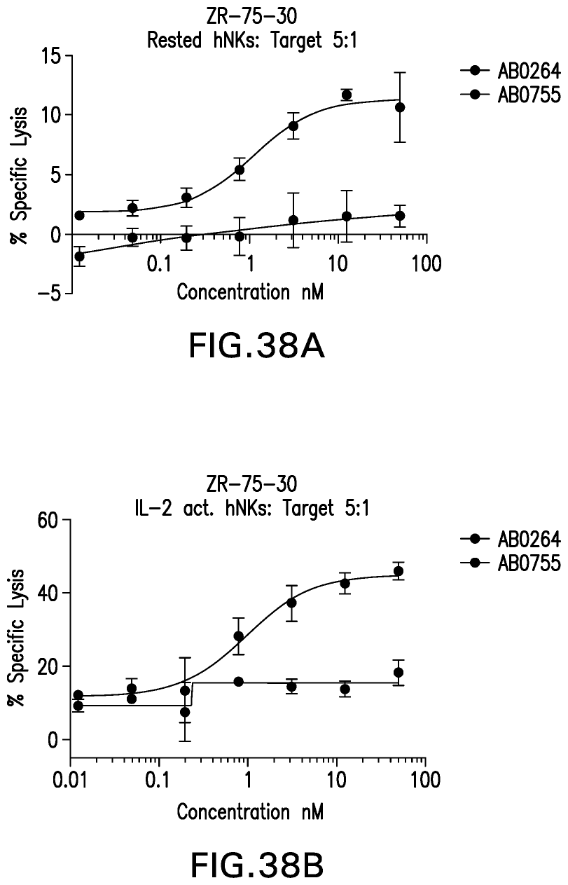

Lysis of ZR-75-30 target cells by rested and IL-2 activated hNK cells derived from the same healthy donor was compared (FIG. 38). EC50 and maximum lysis values are summarized in Table 28. IL-2 activated NK cells showed more potent killing of ZR-75-30 cancer cells compared to rested NK cells. A greater difference was seen in maximum lysis of ZR-75-30 target cells, where AB0264 showed 4-fold higher lysis of target cells with IL-2 activated compared to rested NK cells.

The same analysis was applied to two additional CEACAM-5 expressing human cancer cell lines HPAF-II and LS-174T. Both demonstrated improved EC50 and higher maximum killing when using IL-2 activated NK as an effector comparing to rested NK cells. Killing EC50 and maximum lysis values are summarized in Table 28.

TABLE 28
EC50 and % Max Lysis Values for Rested and Activated NK
Cells Against CEACAM5-Expressing Human Cancer Cell Lines

| | | |
|---|---|---|
| Test | Rested NK | Activated NK |

| | | | | |
|---|---|---|---|---|
| TriNKET | EC50 | Max | EC50 | Max |
| or mAb | (nM) | Killing (%) | (nM) | Killing (%) |

ZR-75-30

| | | | | |
|---|---|---|---|---|
| AB0264 | 1.12 | 11 | 0.96 | 45 |
| AB0755 | ND | ND | ND | ND |

HPAF-II

| | | | | |
|---|---|---|---|---|
| AB0264 | 0.78 | 13 | 0.05 | 52 |
| AB0755 | 0.93 | 6 | 0.12 | 44 |

LS-174T

| | | | | |
|---|---|---|---|---|
| AB0264 | 1.33 | 26 | 0.07 | 74 |
| AB0755 | 0.03 | 11.6 | 0.04 | 62 |

ND = Not determined

TriNKETs Demonstrate Greater Cytolytic Activity Compared to Corresponding mAbs

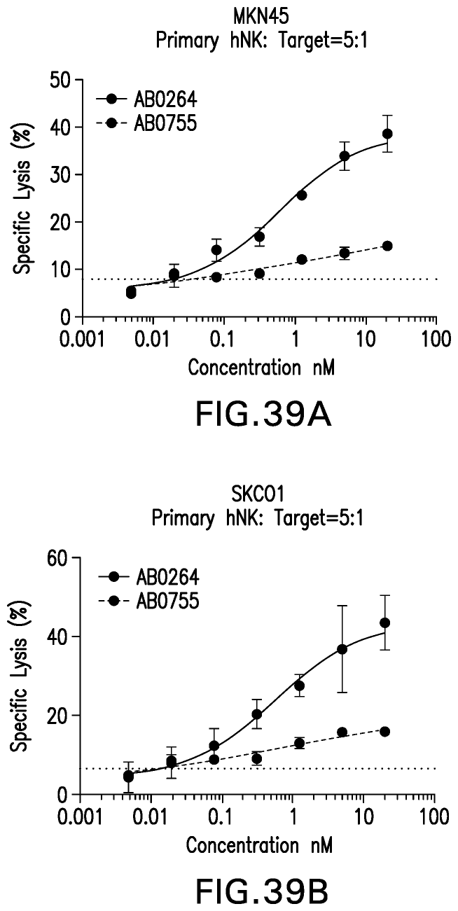

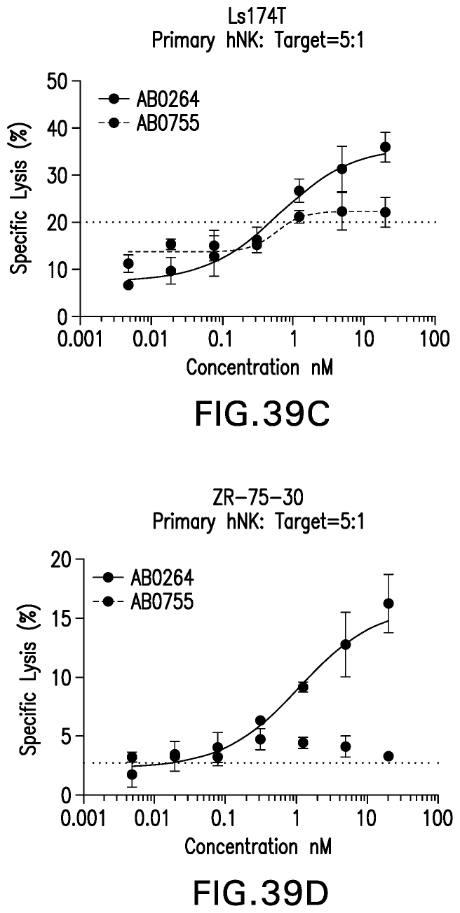

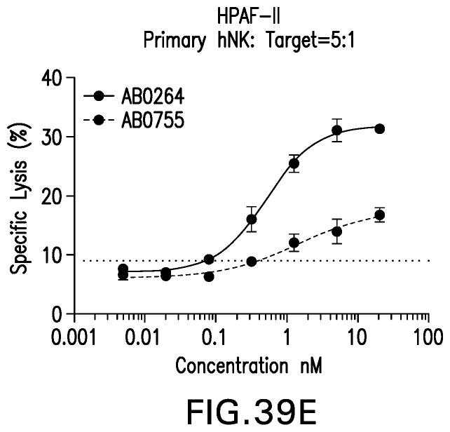

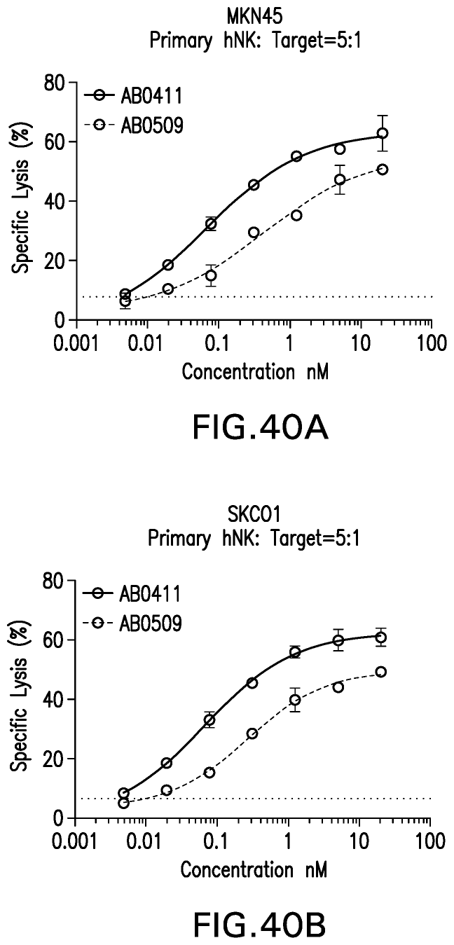

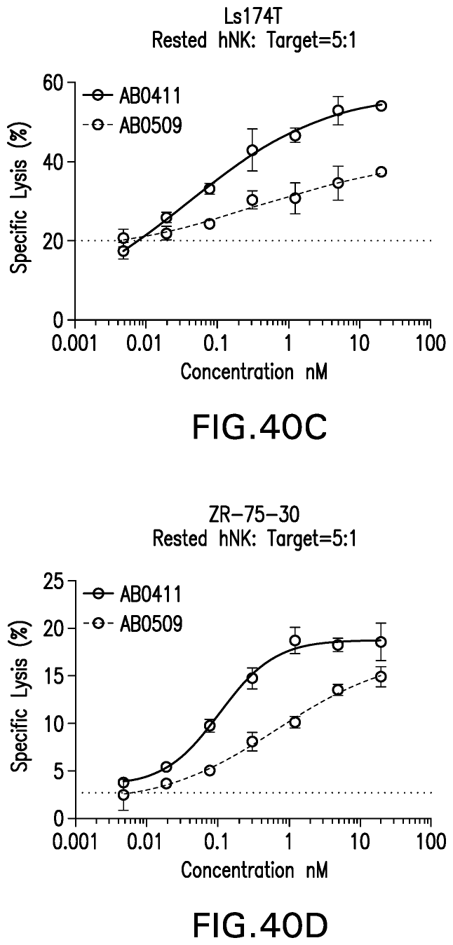

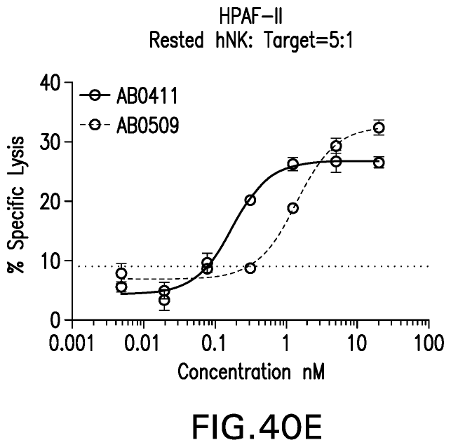

The ability of AB0264 and AB0411 CEACAM-TriNKETs to lyse CEACAM5-expressing human cancer cells was compared with AB0755 and AB0509 respectively using a short-term primary NK cell cytotoxicity assay. AB0755 is a humanized mAb against CEACAM5 with Fab sequence corresponding to scFV anti-CEACAM5 present in AB0264. AB0509 is a humanized mAb against CEACAM5 with Fab sequence corresponding to scFV anti-CEACAM5 present in AB0411. Purified human NK cells were thawed and rested overnight. The following day, rested NKs were co-cultured with labeled (A) MKN-45, (B) SK-CO-1, ((C) LS-174T, (D) ZR-75-30, and (E) HPAF-II target cells for DELFIA assay. Dose-titrations of AB0264 and AB0411 TriNKETs or their corresponding mAb were prepared starting at 20 nM and were added to the co-cultures of human NK cells and target cells. Specific lysis was plotted against the concentration of each TriNKET or mAb and data were fit to a 4-parameter non-linear regression model to generate potency values. AB0264 (FIGS. 39A-39E) and AB0411 (FIGS. 40A-40E) demonstrated high potency in killing five cancer cell lines and outperformed their corresponding mAbs. The results are summarized in Table 29.

TABLE 29
EC50 and % Max Killing Values for AB0264, AB0411 and Corresponding mAbs

| | MKN-45 | | SK-CO-1 | | ZR-75-30 | | HPAF-II | | LS-174T | |
|---|---|---|---|---|---|---|---|---|---|---|
| Test | | Max | | Max | | Max | | Max | | Max |
| TriNKET | EC50 | Killing | EC50 | Killing | EC50 | Killing | EC50 | Killing | EC50 | Killing |
| or mAb | (nM) | (%) | (nM) | (%) | (nM) | (%) | (nM) | (%) | (nM) | (%) |
| AB0264 | 0.60 | 39 | 0.57 | 44 | 1.11 | 16 | 0.51 | 32 | 0.64 | 36 |
| AB0755 | ND | 14 | ND | 22 | ND | 5 | 1.42 | 18 | ND | 22 |
| AB0411 | 0.07 | 63 | 0.07 | 61 | 0.11 | 19 | 0.17 | 27 | 0.04 | 54 |
| AB0509 | 0.43 | 50 | 0.26 | 49 | 0.72 | 15 | 1.41 | 33 | ND | 37 |

ND = Not determined

### NK-Mediated Killing of CEACAM5-Expressing Tumor Cells is Dependent on the Co-Engagement of TriNKET Binding Arms to CD16, NKG2D, and CEACAM5

Variants of AB0264 were generated with mutations in its different binding arms. AB0754 is a CD16-silent variant of AB0264 made to abrogate FcγR binding by introducing mutations into the CH2 domain. AB0752 is an NKG2D-dead variant with mutation in the NKG2D-binding arm. AB0444, an F3′-TriNKET with a palivizumab-based scFv in place of the CEACAM5-binding arm, was generated to abolish binding to CEACAM5 on target cells.

KHYG-1-CD16V cells were rested overnight. The following day, KHYG-1-CD16V cells were co-cultured with labeled MKN-45 target cells for DELFIA assay. Dose-titrations of AB0264 or loss-of-function variants were prepared starting at 20 nM and were added to the co-cultures of KHYG-1-CD16V cells and MKN-45 target cells. Specific lysis was plotted against concentration and data were fit to a 4-parameter non-linear regression model to generate potency values.

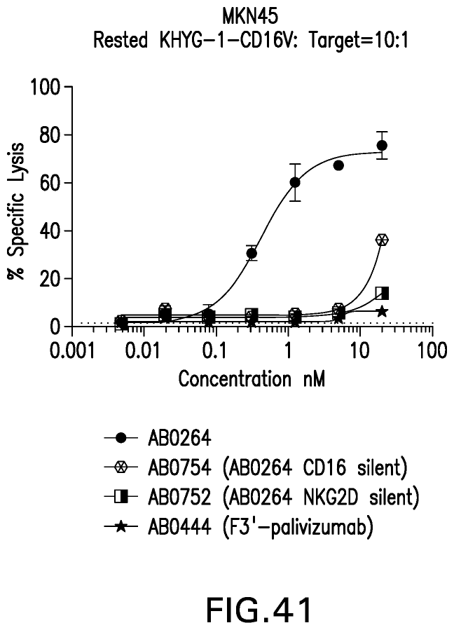

In the absence of target cell binding, no activity was observed with AB0444, demonstrating the contribution of CEACAM5 binding to NK-mediated target cell lysis. AB0754 and AB0752 showed little to no activity, demonstrating the further importance of CD16 and NKG2D binding to the killing activity of AB0264 (FIG. 41).

### IFNγ ELISA Assay

Assay plates were set up as in the DELFIA assay but with a longer incubation time of 48 to 72 hours. Freshly isolated human NK cells were rested overnight. The following day, rested NKs were co-cultured with SK-CO-1 at 10:1 ratio. Dose-titrations of CEACAM5 TriNKETs were prepared starting at 133 nM final concentration at a series of 1:5 dilutions. After incubation, assay plates were briefly spun down and IFNγ in the supernatants from assay wells was quantified by hIFNγ Quantikine® kit (R&D, #SIF50) following manufacture's protocol. Data were fit to a 4-parameter non-linear regression model to generate potency values. EC50 and maximum IFNγ release level were generated by averaging results from three independent NK donors.

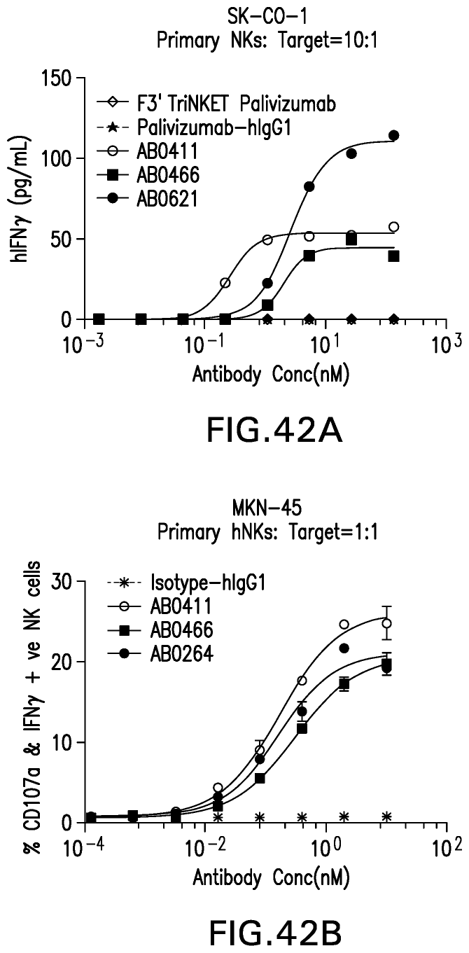

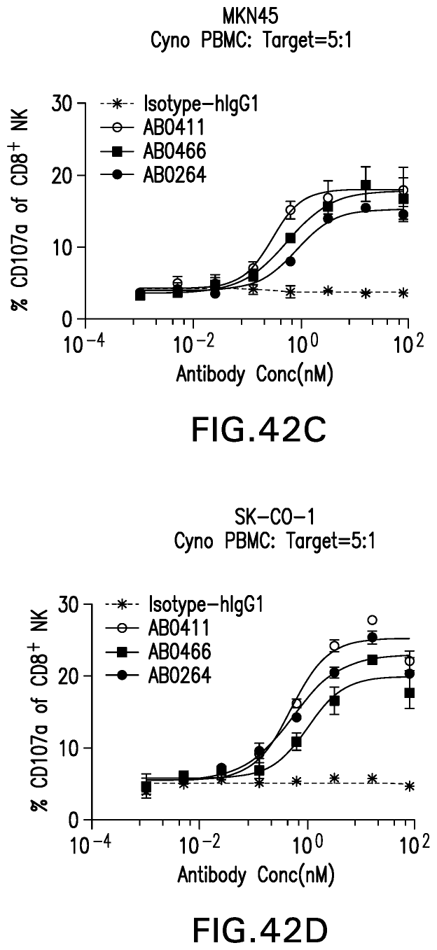

The ability of CEACAM5 TriNKETs to trigger IFNγ production in the co-culture system was evaluated at 48 hours post-treatment. While the control palivizumab-TriNKET did not trigger appreciable amounts of IFNγ, a significant amount of IFNγ was induced by CEACAM5 TriNKETs in a dose-dependent manner (FIG. 42A). The EC50s and maximum induction of IFNγ by TriNKETs are summarized in Table 30.

TABLE 30
EC50 and Maximum Release of IFNγ in Human Primary NKs

| | | Maximum IFNγ |
|---|---|---|
| Test TriNKET | EC50 (nM) | (pg/mL) |
| AB0411 | 0.24 | 38.2 |
| AB0466 | 4.20 | 24.6 |
| AB0621 | 4.03 | 79.3 |

### IFNγ and CD107a Activation Assays

TriNKETs or hIgG1 control were diluted in culture media. CEACAM-5-expressing human cancer cells, rested human primary NK cells, or PBMCs were harvested from culture and resuspended to 1×10⁶ cells/mL in culture media. Recombinant hIL-2 and fluorophore-conjugated anti-CD107a antibody were added to the NK cells or PBMCs for the activation culture. For intracellular cytokine staining, Brefeldin-A (BFA) and monensin were diluted into culture media to block protein transport out of the cell. CEACAM5-expressing MKN-45 tumor cells and primary NK or PBMC effector cells were mixed at a ratio of 1:1. Assay plates were cultured for 4 hours to allow NK cell activation before cells were stained and analyzed by flow cytometry. CD107a and IFNγ staining was analyzed in CD3⁻CD56⁺ populations to assess human NK cell activation. The percentage of IFNγ⁺CD107⁺ NK cells induced was plotted against concentration, and data were fit to a 4-parameter non-linear regression model to generate potency values.

An isotype human IgG1 showed little basal induction of CD107a degranulation or intracellular IFNγ accumulation after four hours. Addition of AB0264 to the co-cultures resulted in robust induction of IFNγ production and CD107a degranulation in a dose-responsive manner (FIG. 42B).

The ability of CEACAM5 TriNKET molecules to enhance activation of cynomolgus NK cells was assessed in a co-culture assay using human cancer cell line with primary cynomolgus PBMCs. The assay using cynomolgus PBMCs was set up in a similar way as the human assay described above. Frozen cynomolgus PBMCs were thawed and rested in culture media at 37° C., 5% CO₂. MKN-45 or SK-CO-1 human cancer cell lines were mixed with rested cyno PBMCs at a 5:1 effector to target cell ratio, together with desired concentrations of TriNKETs or hIgG1 control. BFA, monensin, rhIL-2, and fluorophore conjugated anti-CD107a was added to the PBMCs for the activation culture. Plates were cultured at 37° C., 5% CO₂ for 4 hours before samples were prepared for flow cytometry analysis to measure NK cell CD107a degranulation in the NK and tumor-cell co-culture system. Percentage of CD8⁺ NK cells that were CD107a⁺ was plotted against TriNKET or control concentration, and data were fit to a 4-parameter non-linear regression model to generate potency values.

Cyno-NKG2D expression was found consistently only on CD8⁺ NK cells, as opposed to CD8⁻ NK cells. Thus, a gating strategy using CD45⁺CD14⁻CD20⁻CD3⁻CD8⁺ was applied to define the cynomolgus CD8⁺ NK cells. All CEACAM5-TriNKETs tested showed dose-responsive activity in enhancing degranulation of CD8⁺ NK cells with each of the three cynomolgus PBMC samples tested (FIG. 42 C-D). In contrast, a hIgG1 isotype control showed similar levels of CD107a staining to untreated samples at all concentrations assessed. Table 31 summarizes the potencies and maximum percentage of CD107a degranulation triggered by CEACAM-5 TriNKETs in cynomolgus and human NK cells.

TABLE 31
EC50 and Max Values of CD107a Degranulation
for NK Cells Against CEACAM5⁺ Cancer Cells

| | Human PBMC | | Cyno PBMC | | | |
|---|---|---|---|---|---|---|
| | MKN-45 | | MKN-45 | | SK-CO-1 | |
| Test | EC50 | Max % | EC50 | Max % | EC50 | Max % |
| TriNKET | (nM) | CD107a | (nM) | CD107a | (nM) | CD107a |
| AB0264 | 0.16 | 20.67 | 0.48 | 13.2 | 0.61 | 16.05 |
| AB0411 | 0.17 | 25.33 | 0.26 | 11.49 | 0.47 | 17.40 |
| AB0466 | 0.31 | 19.99 | 1.54 | 13.76 | 0.87 | 14.44 |

### Potency in Short-Term Killing Assay Using Rested Primary Human NK and Patient-Derived Primary Lung Tumor Organoid Lines

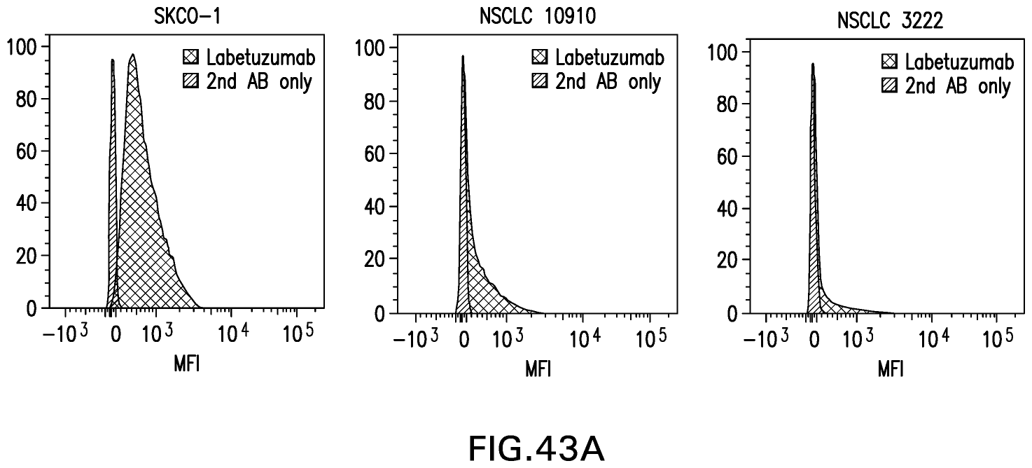

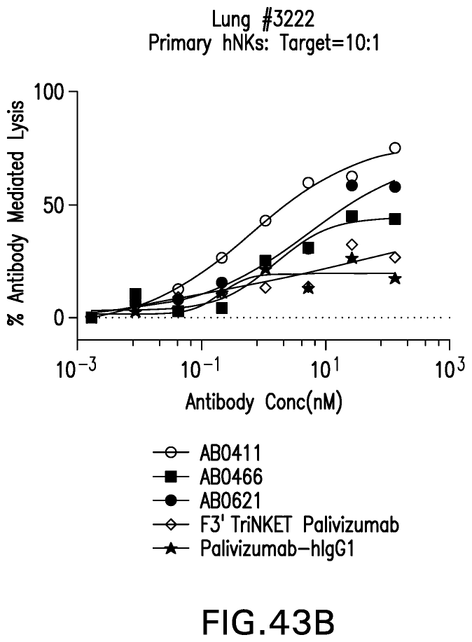

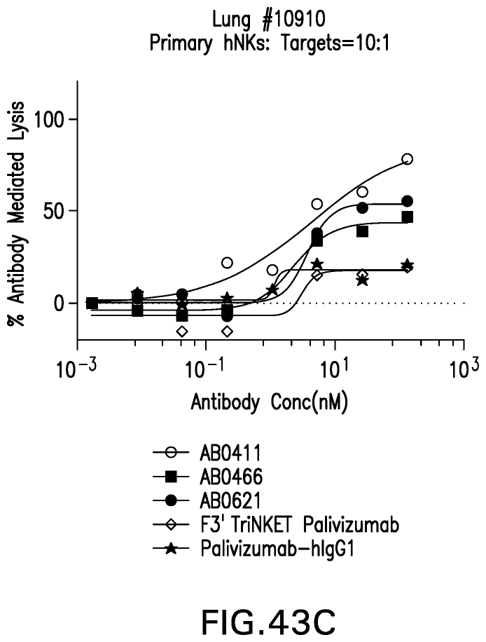

NSCLC 10910 and NSCLC 3222 were two tumor organoid lines derived from primary non-small cell lung cancer (NSCLC) patients. Flow-cytometry analysis with unconjugated anti-CEACAM5 mAb (labetuzumab) and a PE-conjugated secondary antibody demonstrated surface expression of CEACAM5 on these two lines at a much lower level compared to SK-CO-1 (FIG. 43A). The short-term DELFIA assay was used to quantify the ability of CEACAM5 TriNKETs to trigger NK-mediated cytolysis of these two primary NSCLC organoid lines. Freshly isolated human NK cells were rested overnight. The following day, rested NKs were co-cultured with NSCLC 10910 or NSCLC3222 at 10:1 ratio. Dose-titrations of TriNKETs and control molecules were prepared starting at 133 nM final concentration at a series of 1:5 dilutions. Data were fit to a 4-parameter non-linear regression model to generate potency values. Despite the difference in CEACAM5 expression levels, human primary NK cells triggered potent tumor cell lysis in both lines (FIG. 43B-C, Table 32).

TABLE 32
EC50 and % Max lysis Values for Rested NK Cells Against
CEACAM5-Expressing Primary Human Cancer Organoid Lines

| | |
|---|---|
| NSCLC 3222 | NSCLC 10910 |

| | | Max | | Max |
|---|---|---|---|---|
| Test | EC50 | Killing | EC50 | Killing |
| TriNKET | (nM) | (%) | (nM) | (%) |
| AB0411 | 0.61 | 75.25 | 4.34 | 78.16 |
| AB0466 | 1.52 | 43.62 | 1.29 | 47.17 |
| AB0621 | 5.15 | 57.92 | 3.71 | 55.64 |

### Potency in Short-Term Killing Assay Using Activated CD8⁺ T Cells and MKN45

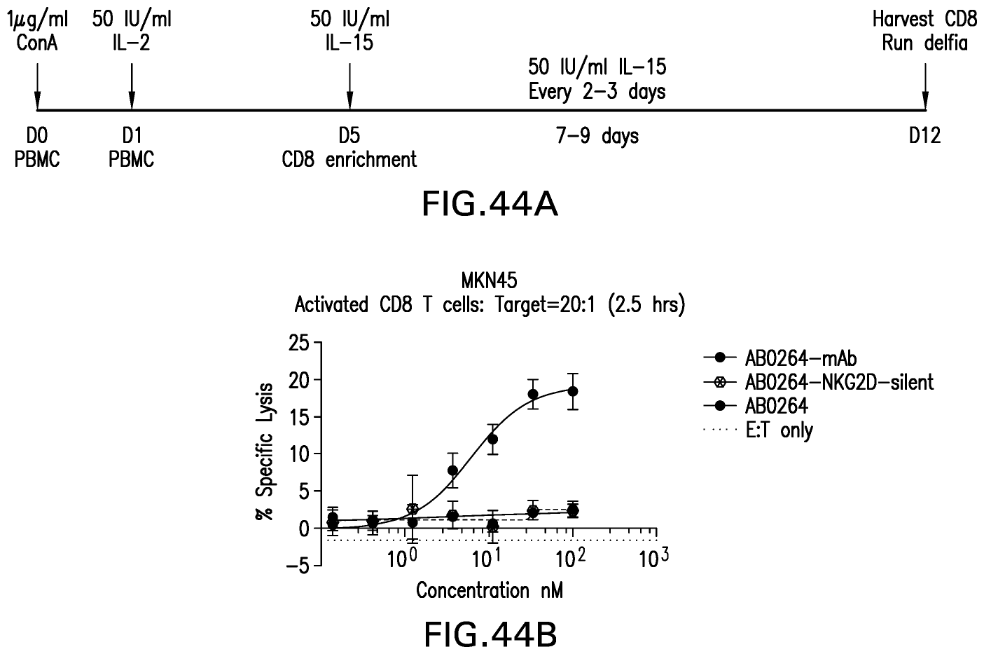

Beyond NK cells, NKG2D is also expressed on cytotoxic T cells. Activated CD8⁺ T cells can be triggered directly by NKG2D stimulation. Cytokine-stimulated CD8 cells were generated using a scheme illustrated in FIG. 44A. In vitro activated human CD8⁺ T cells were co-cultured with MKN-45 cells at a 20:1 E:T ratio. Specific lysis was plotted against concentration and data were fit to a 4-parameter non-linear regression model to generate potency values. Activated T cells showed no basal lysis of target cells. Neither the additional of the corresponding mAb nor the NKG2D-silent variant, which are unable to agonize NKG2D, showed any triggering of T cell activity. In contrast, AB0264 showed a dose-dependent induction in CD8⁺ T cell-mediated cytolysis of MKN-45 target cells (FIG. 44B).

### Example 16. Anti-Tumor Activity of Mouse Surrogate TriNKET mAB0621 in hCEACAM5 Tg Mice with B16F10-hCEACAM5 Tumors

### Transgenic Mice

B6.Cg-Tg(hCEACAM5)2682Wzm/Ieg mice express human CEACAM5 under the control of the human CEACAM5 promoter (Eades-Perner, 1994). Approximately 7- to 12-week-old female heterozygous mice weighing on average 21.7 g were obtained from a breeding colony maintained at Taconic Laboratory (Germantown, NY). These mice contain approximately 2.5 copies per haploid genome of a 33 kb cosmid clone insert containing the complete human CEACAM5 gene and flanking sequences, based on recent re-evaluation of copy number by quantitative PCR.

Distribution of human CEACAM5 expression in these mice is comparable to that in humans, with low level mRNA detected in the colon, ileum, cecum, and stomach. Human CEACAM5 protein expression was observed in the full-thickness in the mucosa of the colon of hCEACAM5 transgenic mice, with the great majority of colonic epithelial cells staining positive. In comparison, CEACAM5 expression in human colon is seen mostly localized to the upper mucosa, particularly along the luminal surface.

### Antibody Reagents and Formulations

A mouse surrogate duobody TriNKET, designated mAB0621, was generated with the human anti-CEACAM5 Fab arm of human TriNKET molecule AB0621 built into a heterodimeric antibody (duobody) with a mouse anti-mouse NKG2D binder clone 13 forming the second Fab arm that is joined together on the mouse IgG2a isotype. Mutations from Genmab DuoBodies were used in the CH3 domains of the mouse IgG2a to form the bispecific duobody TriNKET molecule. An isotype control mouse surrogate duobody TriNKET was similarly made using the synagis anti-human RSV Fab sequence in place of the AB0621 Fab. Mouse surrogate duobody TriNKETs were produced by recombinant cell lines, formulated in 20 mM Na acetate, 9% sucrose, pH5.5, and stored as frozen (−80° C.) stocks.

### Tumor Cell Line Generation

The B16F10 mouse melanoma tumor cell line was engineered to stably express human hCEACAM5 using a pRG-RV 2-5 retroviral vector with no selection. B16F10-hCEACAM5 clone 7-2B11 was been confirmed by IHC to express high levels of hCEACAM5 on tumors grown subcutaneously (SC) in mice and tumor-bearing mice had elevated soluble CEACAM5 levels in their serum. mAB0621 binding to the hCEACAM5-B16F10 cell line was assessed by flow cytometry. mAB0621 showed an EC50 value of 21.8 nM.

### Tumor Cell Line Preparation and Implantation

B16F10-hCEACAM5 clone 7-2B11 cells from a frozen stock were maintained in vitro as a monolayer culture in DMEM medium supplemented with 10% heat inactivated fetal bovine serum (FBS), 1× Glutamine, and 1×MEM non-essential amino acids at 37° C. in an atmosphere of 5% CO₂ in air. Cells growing in an exponential growth phase with 80% of confluence were harvested and washed. 1.5×10⁶ cells were injected subcutaneously (SC) in a 100 μL volume of DMEM basal medium in the dorsal right flank of each mouse.

### Tumor and Body Weight Measurements

Tumors were measured the day before the first dose and twice a week thereafter. Tumor length and width were measured using electronic calipers and tumor volume determined using the formula Volume (mm³)=0.5×Length×Width² where length is the longer dimension. Mice were weighed periodically to monitor general health. Before treatment, mice were weighed and tumors from individual mice were measured. To prevent bias, any outliers by weight or tumor volume were removed and the remaining mice distributed into treatment groups with equivalent mean tumor size. Dosing started when the mean tumor volume in the B16F10-hCEACAM5 tumor-bearing mice reached ˜104 mm³ (range 80-120 mm³), 8 days post implant. Animals were administered duobody TriNKETs as described below.

### Dosing Solution Preparation, Administration, and Analyses

Frozen stocks of the duobody TriNKETs to be tested in the animal model were thawed and transferred to wet ice. Stock solution of each duobody TriNKET was diluted to nominal concentration in the appropriate diluent and dosed immediately. B16F10-hCEACAM5 tumor-bearing hCEACAM5-Tg mice were administered mAB0621 duobody TriNKET, or isotype control duobody TriNKET at a 15, 5, 1.5 or 0.5 mg/kg dose, SC, every 3-4 days for a total of 6 doses. Each treatment group included 15 animals. Post dosing, animals continued to be monitored and tumor volumes were measured twice a week. The antitumor activity of mAB0621 was assessed by two parameters; the percentage of remaining animals at Day 26 after treatment with mAB0621 determined by a Kaplan-Meier analysis, and the measurement of the tumor volume after group assignment. Statistical analysis was performed at Day 26 using log-rank test (*: p<0.05, ***: p<0.001, ****: p<0.0001; n.s.=not significant).

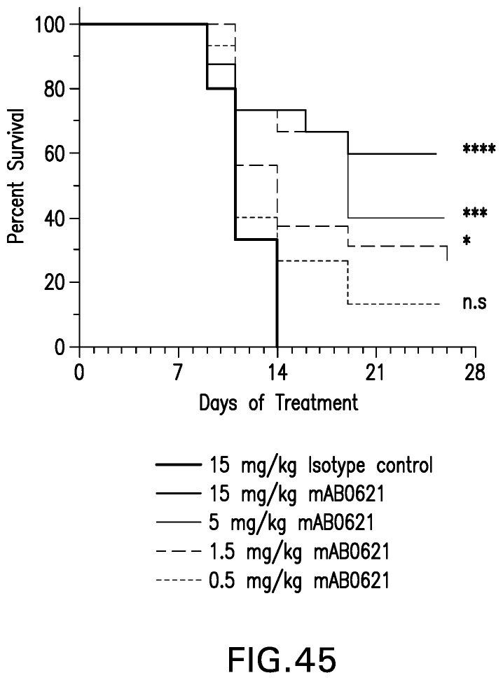

Shown in FIG. 45 are the Kaplan-Meier curves evaluating the percentage of animals remaining over time. Animals bearing a tumor exceeding a volume of 2000 mm³ were euthanized. At Day 26, the number of remaining animals was significantly greater in mice treated with mAB0621 at 15 mg/kg (p<0.0001), at 5 mg/kg (p=0.0004), and at 1.5 mg/kg (p=0.0274) compared to the isotype control group.

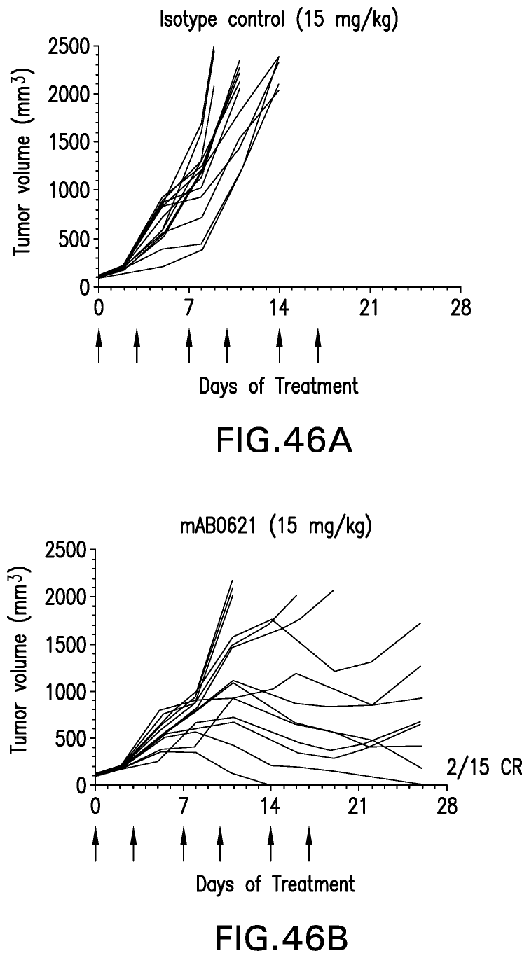

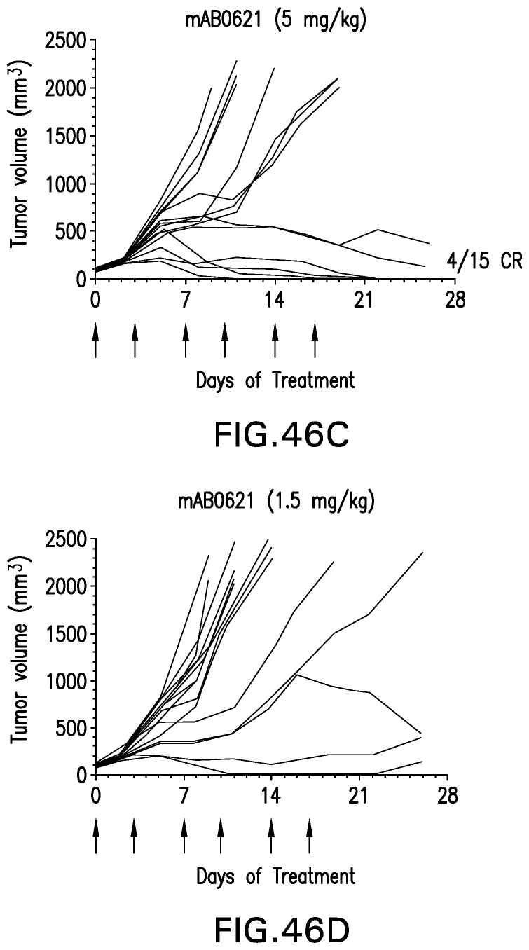

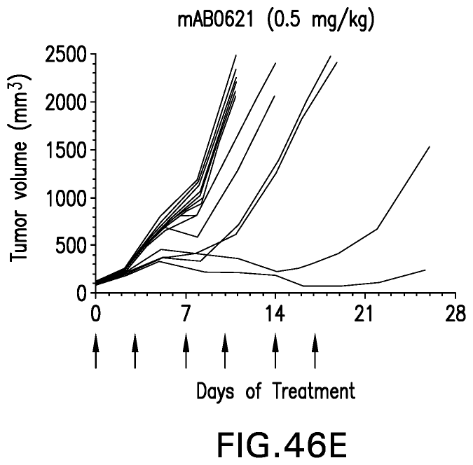

Shown in FIG. 46 are the individual B16F10-hCEACAM5 tumor volumes measured for each animal in the five treatment groups. Tumor volumes were measured twice a week. The comparisons of tumor volumes between the different treatment groups were made collectively over all time points using area under the curve (AUC) as a summary measure for each tumor. The difference between two treatment groups was assessed by Wilcoxon-type non-parametric test for growth curves under dependent right censoring proposed by Vardi et al., 2001.

Shown are individual curves of tumor volumes for B16F10-hCEACAM5 tumor-bearing mice in the hCEACAM5 transgenic model after administration of isotype control at 15 mg/kg (FIG. 46A) or mAB0621 at 15 mg/kg (FIG. 46B), 5 mg/kg (FIG. 46C), 1.5 mg/kg (FIG. 46D), or 0.5 mg/kg (FIG. 46E) through Day 26. Statistically significant tumor regression was observed in the different groups treated with mAB0621 at 15 mg/kg (p=0.00050), and at 5 mg/kg (p=0.00145), and at 0.5 mg/kg (p=0.02870) compared to the control group. The group treated with 1.5 mg/kg of mAB0261 did not show significant tumor regression (p=0.07865). At Day 26, complete tumor regressions (CR) were observed in the 5 mg/kg (4 mice) and 15 mg/kg (2 mice) mAB0621 treatment groups.

### Example 17. Anti-Tumor Activity of Mouse Surrogate TriNKET mAB0621 in Combination with Anti-PD-1 Antibody in hCEACAM5 Tg Mice with B16F10-hCEACAM5 Tumors

### Transgenic Mice

B6.Cg-Tg(hCEACAM5)2682Wzm/Ieg mice are described in Example 16. Approximately 8- to 12-week-old female heterozygous mice weighing on average 23.2 g were obtained from a breeding colony maintained at Taconic Laboratory (Germantown, NY).

### Antibody Reagents and Formulations

The mAB0621 mouse surrogate duobody TriNKET and isotype control TriNKET were described in Example 16. Mouse surrogate duobody TriNKETs and anti-murine PD-1 mouse IgG1 antibody (muDX400) were produced by recombinant cell lines, formulated in 20 mM Na acetate, 9% sucrose, pH5.5, and stored as frozen (−80° C.) stocks.

### Tumor Cell Line, Preparation, and Implantation

The B16F10-hCEACAM5 clone 7-2B11, as well as the culture, preparation, and injection of these cells into mice were as described in Example 16.

### Tumor Measurements and Body Weights

Tumors were measured the day before the first dose and twice a week thereafter. Tumor length and width were measured using electronic calipers and tumor volume determined using the formula Volume (mm³)=0.5×Length×Width² where length is the longer dimension. Mice were weighed periodically to monitor general health. Before treatment, mice were weighed and tumors from individual mice were measured. To prevent bias, any outliers by weight or tumor volume were removed and the remaining mice distributed into treatment groups with equivalent mean tumor size. Dosing started when the mean tumor volume in the B16F10-hCEACAM5 tumor-bearing mice reached ˜237 mm³ (range 220-270 mm³), 7 days post implant, Animals were administered duobody TriNKETs as described below.

### Dosing Solution Preparation and Administration

Frozen stocks of the duobody TriNKETs or muDX400 anti-PD-1 antibody to be tested in the animal model were thawed and transferred to wet ice. Stock solution of each duobody TriNKET was diluted to nominal concentration in the appropriate diluent and dosed immediately.

### Dosing and Results

B16F10-hCEACAM5 tumor-bearing hCEACAM5-Tg mice were administered 5 mg/kg doses of either control isotype antibodies or mAB0621 or anti-PD1 muDX400 as single agents or as combination treatments, SC, every 3-4 days for a total of 6 doses. Each treatment group included 15 animals. Post dosing, animals continued to be monitored and tumor volumes were measured twice a week. The antitumor activity of mAB0621 was assessed by two parameters; the percentage of remaining animals at Day 37 after treatment determined by a Kaplan-Meier analysis, and the measurement of the tumor volume after group assignment. Statistical analysis was performed at Day 37 using log-rank test.

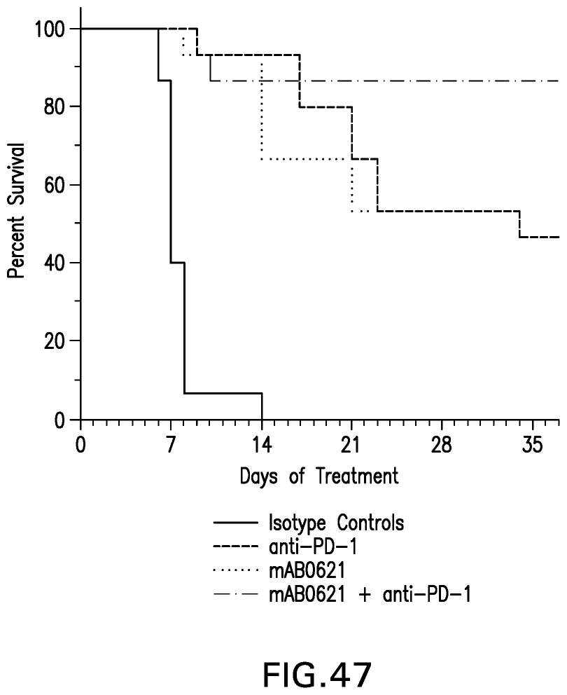

Shown in FIG. 47 are the Kaplan-Meier curves evaluating the percentage of animals remaining over time. Animals bearing a tumor exceeding a volume of 1800 mm³ were euthanized. At Day 37, the number of remaining animals was significantly greater in mice treated with the combination of mAB0621 with anti-PD-1 muDX400 compared to isotype controls (p<0.0001) and compared to anti-PD-1 single agent treatment (p=0.0356), and compared to mAB0621 single agent treatment (p=0.0368).

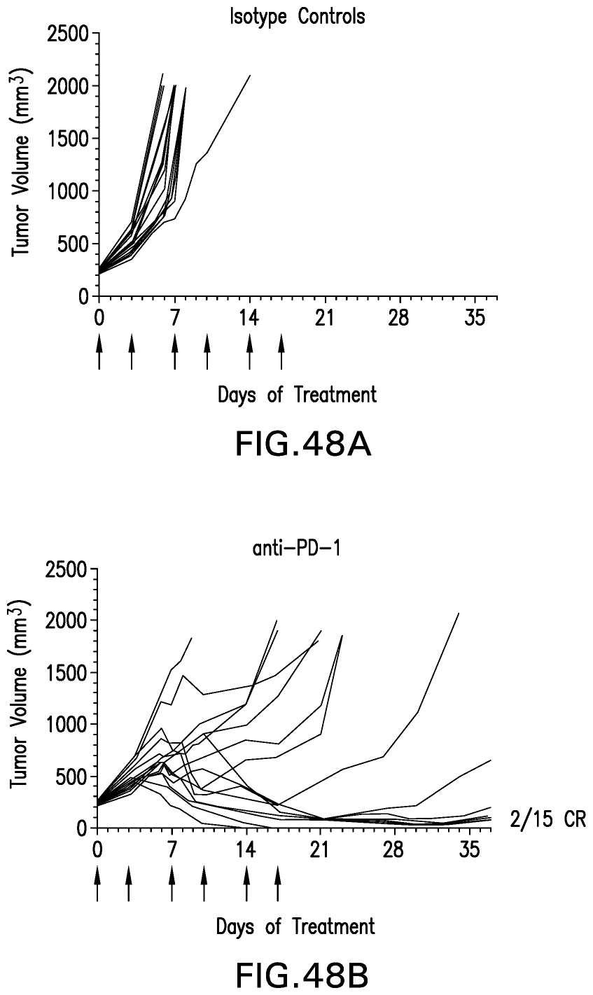

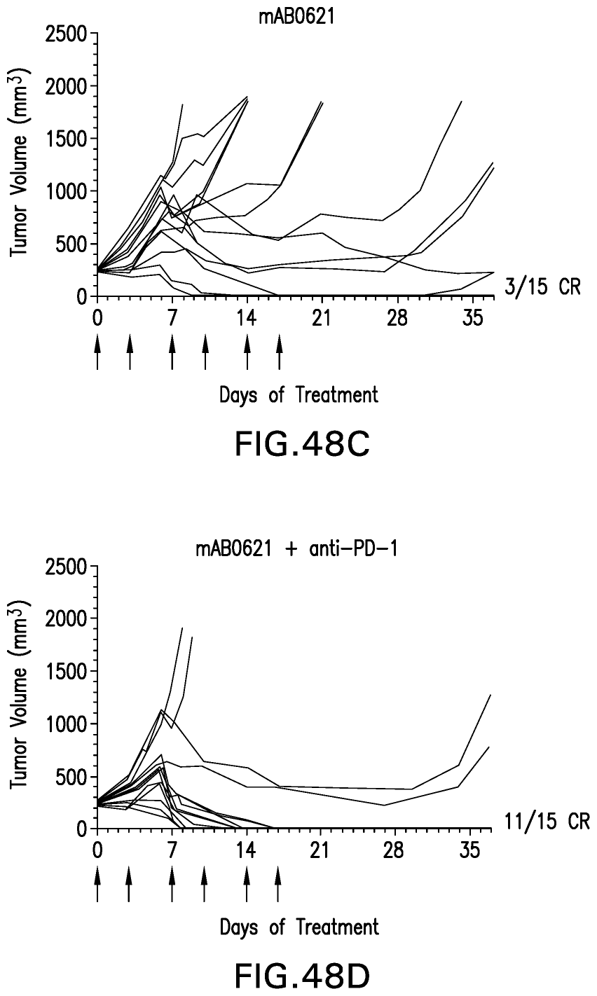

Shown in FIG. 48 are the individual B16F10-hCEACAM5 tumor volumes measured for each animal of the 4 treatment groups. Tumor volumes were measured twice a week. The comparisons of tumor volume between the different treated groups were made collectively over all time points using AUC as a summary measure for each tumor. The difference between two treatment groups was assessed by Wilcoxon-type non-parametric test for growth curves under dependent right censoring proposed by Vardi et al., 2001.

The combination treatment showed statistically significant tumor regression compared to isotype controls (p<0.0001) and compared to the single agent treatment groups, mAB0621 (p=0.0270) and anti-PD-1 (p=0.00490) respectively. Shown are individual curves of tumor volumes for B16F10-hCEACAM5 tumor-bearing mice in the hCEACAM5 transgenic model after administration of isotype control at 5 mg/mg (FIG. 48A) or anti-PD-1 muDX400 at 5 mg/kg (FIG. 48B), or mAB0621 at 5 mg/kg (FIG. 48C), or combination of mAB0621 and anti-PD-1 muDX400 at 5 mg/kg (FIG. 48D) through Day 37. At Day 37, complete tumor regressions were observed with combination treatment mAB0621 with anti-PD-1 (II mice), with anti-PD-1 single agent treatment (2 mice), and with mAB0621 single agent treatment (3 mice).

### Example 18. Pharmacokinetics of AB0264 and AB0411 in Cynomolgus Monkeys

The PK of AB0264 and AB0411 were studied in biologics naïve cynomolgus monkeys. AB0264 and AB0411 were obtained from internal source as frozen (−80° C.) stock. The dosing solution was transferred from nominal −80° C. to nominal 4° C. the night before the dose day. The dosing solution was allowed to come to room temperature for at least 1 hour prior to dose administration and was inverted 5-10 times to ensure the formulation was uniformly mixed before being transferred from the tubes to the syringes.

PK of AB0264 were studied at 0.1, 1, 10 mg/kg doses to understand target mediated drug disposition because of its cross-reactivity to cyno CEACAM5. Six cynomolgus monkeys were divided into three dose groups with 1 male and 1 female in each group. AB0411 does not cross-react with cynomolgus monkey CEACAM5. PK of AB0411 was therefore studied at 10 mg/kg dose only with two female and two male cynomolgus monkeys. On day 0, these cynomolgus monkeys were dosed with AB0264 or AB0411 respectively through intravenous administration.

Serum samples were collected at indicated timepoints up to 14 days post dosing. Drug concentrations were measured by two ligand binding assays. Free drug was measured using recombinant human CEACAM5 and anti-hNKG2D arm anti-ids antibody pair. Total drug was measured with anti-human Fc and anti-human IgG antibody pair.

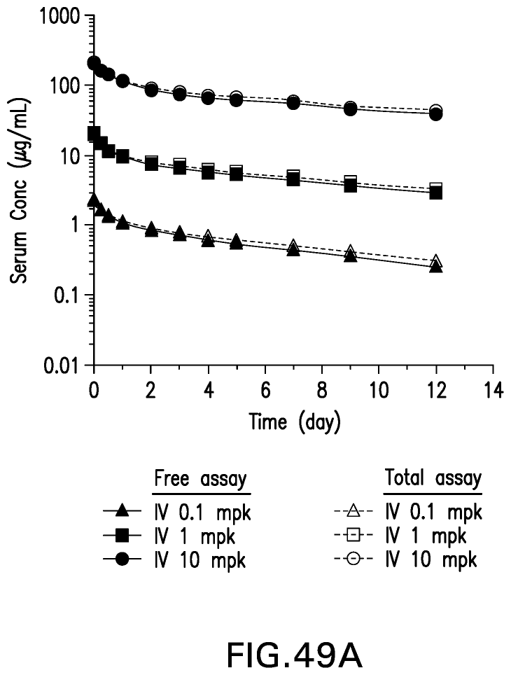

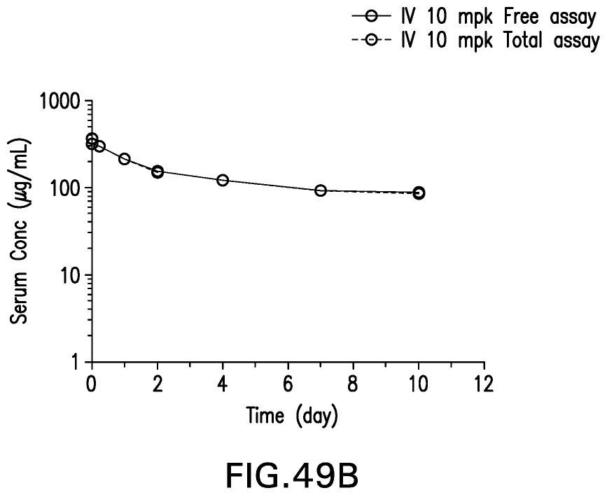

The concentration-time PK profile of the free drug and total drug is plotted as average concentration in each group versus time (AB0264 PK profile in FIG. 49A, and AB0411 PK profile in FIG. 49B). Both AB0264 and AB0411 showed antibody like PK in cynomolgus monkeys. They were stable in vivo with similar exposure of free drug and total drug within the study duration. No gender differences in exposure were observed. For AB0264, serum exposure was linear from 0.1 mg/kg through 10 mg/kg doses.

### Example 19. Pharmacokinetics in hCEACAM5 Transgenic Mice

The PK of AB0621, AB0411 and AB0466 were studied. To allow assessment of the impact of human CEACAM5 expression on TriNKET PK, studies were carried out in the female heterozygous B6.Cg-Tg(hCEACAM5)2682Wzm/Ieg mouse line described in Example 16. Dosing solution of AB0621, AB0411, and AB0466 were prepared following the same procedure as described in the cynomolgus monkey PK studies above.

On day 0, hCEACAM5 transgenic mice were dosed with AB0621, AB0411, and AB0466 at 1 mg/kg and 10 mg/kg doses, respectively, through intravenous administration. Serum samples were collected at indicated timepoints up to 14 days post dosing. Drug concentrations were measured by two ligand binding assays. Free drug was measured using recombinant human CEACAM5 and anti-hNKG2D arm anti-ids antibody pair. Total drug was measured with anti-human Fc and anti-human IgG antibody pair.

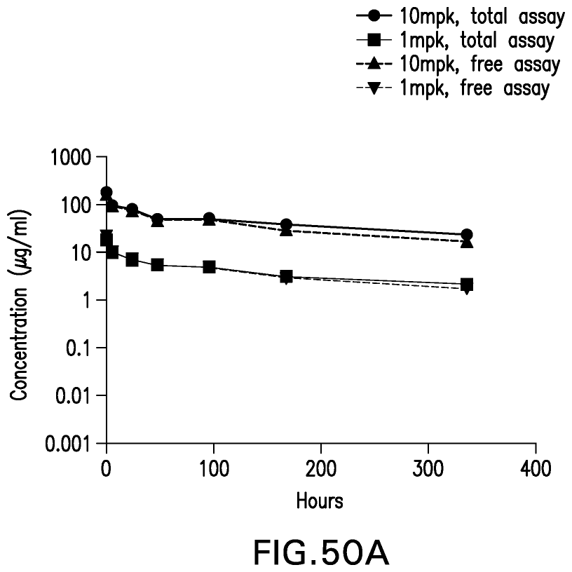

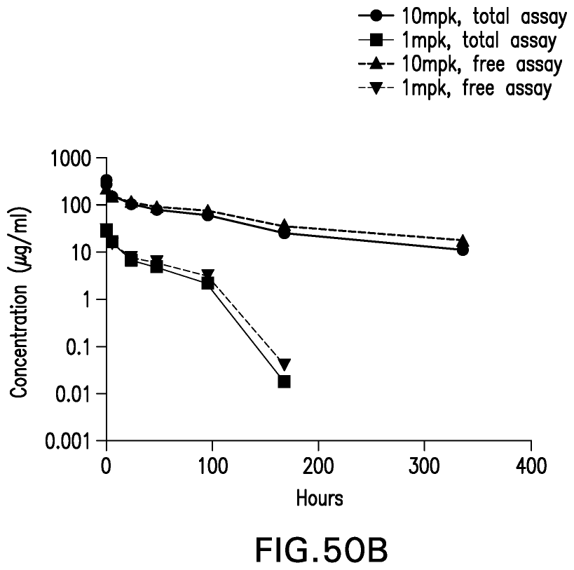

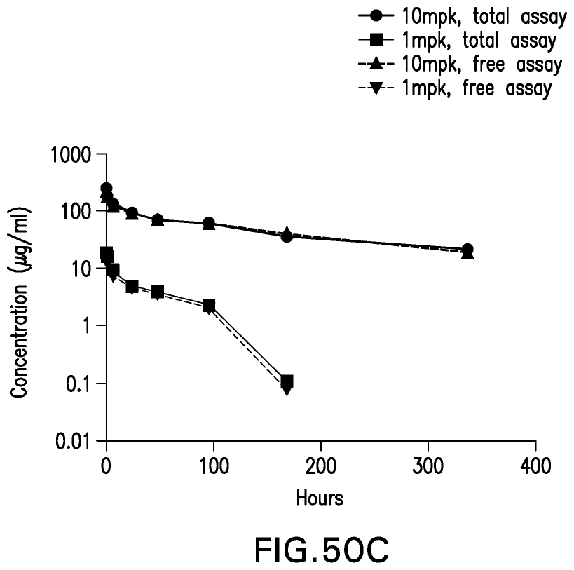

The concentration-time PK profile of AB0621 (FIG. 50A), AB0411 (FIG. 50B), and AB0466 (FIG. 50C) are plotted as average concentration in each group versus time. AB0621, AB0411 and AB0466 showed antibody like PK profile in hCEACAM5 transgenic mice. The exposure of total drug and free drug were aligned for all three tested TriNKETs, which indicates they are stable in vivo. The PK of AB0621 was linear from 1 mg/kg through 10 mg/kg. AB0411 and AB0466 showed non-linear PK from 1 mg/kg through 10 mg/kg, likely due to hCEACAM5 mediated drug disposition at 1 mg/kg dose.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A protein comprising:
(a) a first antigen-binding site that binds NKG2D;
(b) a second antigen-binding site that binds CEACAM5; and
(c) a third antigen-binding site, or an antibody Fc domain or a portion thereof, that binds CD16, wherein:
the second antigen-binding site that binds CEACAM5 comprises a heavy-chain variable domain (VH) comprising a complementarity-determining region (CDR) 1 (CDRH1), CDRH2, and CDRH3, and a light-chain variable domain (VL) comprising a CDR 1 (CDL1), CDRL2, and CDRL3, wherein:
(i) CDRH1 comprises an amino acid sequence selected from the group consisting of: SEQ ID NOs: 3 and 102;
(ii) CDRH2 comprises an amino acid sequence selected from the group consisting of: SEQ ID NOs: 37, 104, and 718;
(iii) CDRH3 comprises an amino acid sequence selected from the group consisting of: SEQ ID NOs: 6, 38, and 105;
(iv) CDRL1 comprises an amino acid sequence selected from the group consisting of: SEQ ID NOs: 7, 40, and 107;
(v) CDRL2 comprises an amino acid sequence selected from the group consisting of: SEQ ID NOs: 8, 41, and 108; and
(vi) CDRL3 comprises an amino acid sequence selected from the group consisting of: SEQ ID NOs: 9, 42, and 109.

2. The protein of claim 1, wherein:
(a) the CDRH1, CDRH2, and CDRH3 of the second antigen-binding site are:
(i) SEQ ID NOs: 3, 37, and 38, respectively;
(ii) SEQ ID NOs: 3, 718, and 6, respectively; or
(iii) SEQ ID NOs: 102, 104, and 105, respectively; and
(b) the CDRL1, CDRL2, and CDRL3 of the second antigen-binding site are:
(i) SEQ ID NOs: 7, 8, and 9, respectively;
(ii) SEQ ID NOs: 40, 41, and 42, respectively; or
(iii) SEQ ID NOs: 107, 108, and 109, respectively.

3. The protein of claim 2, wherein the CDRH1, CDRH2, CDRH3, CDRL1, CDRL2, and CDRL3 are:
(i) SEQ ID NOs: 3, 37, 38, 40, 41, and 42, respectively;
(ii) SEQ ID NOs: 3, 718, 6, 7, 8, and 9, respectively; or
(iii) SEQ ID NOs: 102, 104, 105, 107, 108, and 109, respectively.

4. The protein of claim 1, wherein:
the VH comprises an amino acid sequence selected from the group consisting of: SEQ ID NOs: 704, 708, 711, and 715; and
the VL comprises an amino acid sequence selected from the group consisting of: SEQ ID NOs: 591, 705, 712, and 716.

5. The protein of claim 1, wherein the VH and VL are:
(i) SEQ ID NOs: 704 and 705, respectively;
(ii) SEQ ID NOs: 708 and 591, respectively;
(iii) SEQ ID NOs: 711 and 712, respectively; or
(iv) SEQ ID NOs: 715 and 716, respectively.

6. The protein of claim 1, wherein the second antigen-binding site is a single-chain variable fragment (scFv), wherein the scFv comprises an amino acid sequence selected from the group consisting of: SEQ ID NOs:703, 707, 710, and 714.

7-14. (canceled)

15. The protein of claim 6, wherein the scFv is linked to an antibody Fe domain or a portion thereof that binds CD16, via a hinge comprising Ala-Ser or Gly-Ser, optionally wherein the hinge comprises an amino acid sequence Thr-Lys-Gly.

16-18. (canceled)

19. The protein of claim 6, wherein the heavy chain variable domain of the scFv is linked to the light chain variable domain of the scFv via a flexible linker, optionally wherein the flexible linker comprises (G4S)4.

20-24. (canceled)

25. The protein of claim 1, wherein the first antigen-binding site that binds NKG2D comprises a VH comprising a CDRH1, CDRH2, and CDRH3 comprising the amino acid sequences of SEQ ID NOs: 495, 496, and 510, respectively; and a VL comprising a CDRL1, CDRL2, and CDRL3 comprising the amino acid sequences of SEQ ID NOs:530, 224, and 499, respectively.

26. The protein of claim 25, wherein the VH of the first antigen-binding site comprises an amino acid sequence at least 90% identical to SEQ ID NO:508, and the VL of the first antigen-binding site comprises an amino acid sequence at least 90% identical to SEQ ID NO:493, or
wherein the VH of the first antigen-binding site comprises the amino acid sequence of SEQ ID NO:508, and the VL of the first antigen-binding site comprises the amino acid sequence of SEQ ID NO:493.

27-28. (canceled)

29. The protein of claim 1, wherein the antibody Fc domain or the portion thereof comprises an amino acid sequence at least 90% identical to SEQ ID NO:531, wherein at least one polypeptide chain of the antibody Fc domain or the portion thereof comprises one or more mutations, relative to SEQ ID NO:531, at one or more positions selected from the group consisting of: Q347, Y349, L351, S354, E356, E357, K360, Q362, S364, T366, L368, K370, N390, K392, T394, D399, 5400, D401, F405, Y407, K409, T411, and K439, numbered according to the EU numbering system.

30-32. (canceled)

33. The protein of claim 29, wherein one polypeptide chain of the antibody Fc domain or the portion thereof comprises K360E and K409W substitutions relative to SEQ ID NO:531; and the other polypeptide chain of the antibody Fc domain or the portion thereof comprises Q347R, D399V and F405T substitutions relative to SEQ ID NO:531, numbered according to the EU numbering system, and
wherein one polypeptide chain of the antibody heavy chain constant region comprises a Y349C substitution relative to SEQ ID NO:531; and the other polypeptide chain of the antibody heavy chain constant region comprises an S354C substitution relative to SEQ ID NO:531, numbered according to the EU numbering system.

34. (canceled)

35. A protein comprising:
(a) a first polypeptide comprising the amino acid sequence of SEQ ID NO:549;
(b) a second polypeptide comprising the amino acid sequence of SEQ ID NO:550; and
(c) a third polypeptide comprising an amino acid sequence selected from the group consisting of: SEQ ID NOs:702, 706, 709, and 713.

36. An isolated nucleic acid molecule, or a plurality of isolated nucleic acid molecules, encoding the protein of claim 1, or an expression vector or a plurality of expression vectors comprising the isolated nucleic acid molecule, or the plurality of isolated nucleic acid molecules, encoding the protein of claim 1.

37-38. (canceled)

39. A host cell comprising the expression vector or the plurality of expression vectors of claim 36, optionally wherein the host cell is a Chinese hamster ovary (CHO) cell.

40. (canceled)

41. A method of producing a protein comprising:
(a) a first antigen-binding site that binds NKG2D;
(b) a second antigen-binding site that binds CEACAM5; and
(c) a third antigen-binding site, or an antibody Fc domain or a portion thereof, that binds CD16;
wherein the method comprises:
(i) providing a host cell of claim 39;
(ii) cultivating the host cell in a medium under conditions suitable for expressing the protein; and
(iii) isolating the protein from the medium.

42. (canceled)

43. A pharmaceutical composition comprising a protein of claim 1 and a pharmaceutically acceptable carrier.

44. A method of enhancing tumor cell death in a subject with cancer, the method comprising exposing the tumor cell and a natural killer cell to an effective amount of the pharmaceutical composition of claim 43, optionally wherein the cancer is selected from the group consisting of: gastrointestinal cancer, colorectal cancer, pancreatic cancer, non-small cell lung cancer, and esophageal cancer.

45. A method of treating cancer, the method comprising administering an effective amount of the pharmaceutical composition of claim 43 to a patient in need thereof, optionally wherein the cancer is selected from the group consisting of: gastrointestinal cancer, colorectal cancer, pancreatic cancer, non-small cell lung cancer, and esophageal cancer.

46-49. (canceled)

50. A combination therapy for treating cancer comprising administering to a subject in need thereof a therapeutically effective amount of the protein of of claim 1 and a therapeutically effective amount of a checkpoint inhibitor, optionally wherein the checkpoint inhibitor is an anti-PD1 antibody or an anti-PD-L1 antibody, optionally wherein the checkpoint inhibitor is pembrolizumab.

51-52. (canceled)

53. A protein comprising an antigen-binding site that binds CEACAM5, wherein the antigen-binding site comprises a VH comprising a CDRH1, CDRH2, and CDRH3, and a VL comprising a CDRL1, CDRL2, and CDRL3, wherein:
(i) CDRH1 comprises an amino acid sequence selected from the group consisting of: SEQ ID NOs: 3 and 102;
(ii) CDRH2 comprises an amino acid sequence selected from the group consisting of: SEQ ID NOs: 37, 104, and 718;
(iii) CDRH3 comprises an amino acid sequence selected from the group consisting of: SEQ ID NOs: 6, 38, and 105;
(iv) CDRL1 comprises an amino acid sequence selected from the group consisting of: SEQ ID NOs: 7, 40, and 107;
(v) CDRL2 comprises an amino acid sequence selected from the group consisting of: SEQ ID NOs: 8, 41, and 108; and
(vi) CDRL3 comprises an amino acid sequence selected from the group consisting of: SEQ ID NOs: 9, 42, and 109.

54. The protein of claim 53, wherein:
(a) the CDRH1, CDRH2, and CDRH3 are:
(i) SEQ ID NOs: 3, 37, and 38, respectively;
(ii) SEQ ID NOs: 3, 718, and 6, respectively; or
(iii) SEQ ID NOs: 102, 104, and 105, respectively; and
(b) the CDRL1, CDRL2, and CDRL3 are:
(i) SEQ ID NOs: 7, 8, and 9, respectively;
(ii) SEQ ID NOs: 40, 41, and 42, respectively; or
(iii) SEQ ID NOs: 107, 108, and 109, respectively.

55. The protein of claim 54, wherein the CDRH1, CDRH2, CDRH3, CDRL1, CDRL2, and CDRL3 are:
(i) SEQ ID NOs: 3, 37, 38, 40, 41, and 42, respectively;
(ii) SEQ ID NOs: 3, 718, 6, 7, 8, and 9, respectively; or
(iii) SEQ ID NOs: 102, 104, 105, 107, 108, and 109, respectively.

56. The protein of any one of claim 53, wherein:
the VH comprises an amino acid sequence selected from the group consisting of: SEQ ID NOs: 704, 708, 711, and 715; and
the VL comprises an amino acid sequence selected from the group consisting of: SEQ ID NOs: 591, 705, 712, and 716.

57. The protein of claim 53, wherein the VH and VL are:
(i) SEQ ID NOs: 704 and 705, respectively;
(ii) SEQ ID NOs: 708 and 591, respectively;
(iii) SEQ ID NOs: 711 and 712, respectively; or
(iv) SEQ ID NOs: 715 and 716, respectively.

58-77. (canceled)

78. An isolated nucleic acid molecule encoding the protein of claim 53, or
an expression vector comprising an isolated nucleic acid molecule encoding the protein of claim 53.

79. (canceled)

80. A host cell comprising the expression vector of claim 78, optionally wherein the host cell is a Chinese hamster ovary (CHO) cell.

81. (canceled)

82. A method of producing a protein comprising:
(a) providing the host cell of claim 80;
(b) cultivating the host cell in a medium under conditions suitable for expressing the protein; and
(c) isolating the protein from the medium.

83. A pharmaceutical composition comprising a protein of claim 53 and a pharmaceutically acceptable carrier.

84. (canceled)

85. A method of treating cancer, the method comprising administering an effective amount of the pharmaceutical composition of claim 83 to a patient in need thereof, optionally wherein the cancer is selected from the group consisting of: gastrointestinal cancer, colorectal cancer, pancreatic cancer, non-small cell lung cancer, and esophageal cancer.

86-88. (canceled)

89. A combination therapy for treating cancer comprising administering to a subject in need thereof a therapeutically effective amount the pharmaceutical composition of claim 83 and a therapeutically effective amount of a checkpoint inhibitor, optionally wherein the checkpoint inhibitor is an anti-PD1 antibody or an anti-PD-L1 antibody, optionally wherein the checkpoint inhibitor is pembrolizumab.

90-91. (canceled)
